SEQUENCE LISTING (1238 sequences)

SEQ ID NO: 1 - DNA, 1318 bp, synthetic construct
  misc_feature 1..1318: note = DNA sequence encoding fusion protein comprising transferrin receptor 1 linked to ten human Ara h1 and 2 epitope encoding nucleotide sequences
  source 1..1318: mol_type = other DNA
atgatggatcaggcgcgcagcgcgtttagcaacctgtttggcggcgaaccgctgagctatacccgctttagcctggcgcgccaggtggatggcgataacagccatgtggaaatgaaactggcggtggatgaagaagaaaacgcggataacaacaccaaagcgaacgtgaccaaaccgaaacgctgcagcggcagcatttgctatggcaccattgcggtgattgtgttttttctgattggctttatgattggctatctgggctattgcaaaggcgtggaaccgaaaaccgaatgcgaacgcctggcgggcaccgaaagcccggtgcgcgaagaaccgggcgaagattttccggcgggcggcggcggcagccgcgtgcgccgcggcggcggcggcagctggcagaacctgcagaaccatcgcattgtgcagattgaagcgaaaccgaacaccctggtgggcggcggcggcagccgcgtgcgccgcggcggcggcggcagctggagcacccgcagcagcgaaaacaacgaaggcgtgattgtgaaagtgagacaaagaaggcggcggcggcagccgcgtgcgccgcggcggcggcggcagcaacaactttggcaaactgtttgaagtgaaaccggataaaaaaaacccgcagctgcagggcggcggcggcagccgcgtgcgccgcggcggcggcggcagcgtggaaattaaagaaggcgcgctgatgctgcctgattttaacagcaaagcgggcggcggcggcagccgcgtgcgccgcggcggcggcggcagcgcgctgatgctgccgcattttaacagcaaagctatggtgattgtggtggtgggcggcggcggcagccgcgtgcgccgcggcggcggcggcagcaaagcgatggtgattgtggtggtgaacaaaggcaccggcaacctggaactggtggcggtgggcggcggcggcagccgcgtgcgccgcggcggcggcggcagcggcgatgtgtttattatgccggcggcgcatccggtggcgattaacgcgagcagcggcggcggcggcagccgcgtgcgccgcggcggcggcggcagcagccagctggaacgcgcgaacctgcgcccgagcgaacagcatctgatgggcggcggcggcagcdgcgtgcgccgcggcggcggcggcagcgaactgaacgaatttgaaaacaaccagcgcagcatgagcgaagcgctgcagggcggcggcggcagccgcgtgcgccgcggcggcggcggcagccgcgaactgcgcaacctgccgcagcagagcggcctgcgcgcgcgcgtgcgccgc

SEQ ID NO: 2 - DNA, 354 bp, Homo sapiens
  source 1..354: mol_type = other DNA
atgatggatcaggcgcgcagcgcgtttagcaacctgtttggcggcgaaccgctgagctatacccgctttagcctggcgcgccaggtggatggcgataacagccatgtggaaatgaaactggcggtggatgaagaagaaaacgcggataacaacaccaaagcgaacgtgaccaaaccgaaacgctgcagcggcagcatttgctatggcaccattgcggtgattgtgttttttctgattggctttatgattggctatctgggctattgcaaaggcgtggaaccgaaaaccgaatgcgaacgcctggcgggcaccgaaagcccggtgcgcgaagaaccgggcgaagattttccggcg

SEQ ID NO: 3 - protein, 11 aa, Homo sapiens
CGGKLGRKRTL

SEQ ID NO: 4 - protein, 12 aa, synthetic construct
  REGION 1..12: note = Peptide targeting sequence
CGGKLGRKRYLR

SEQ ID NO: 5 - protein, 5 aa, synthetic construct
  REGION 1..5: note = Peptide linker
GGGGS

SEQ ID NO: 6 - protein, 4 aa, synthetic construct
  REGION 1..4: note = Peptide linker
GGGG

SEQ ID NO: 7 - DNA, 45 bp, synthetic construct
  misc_feature 1..45: note = DNA encoding peptide linker
  source 1..45: mol_type = other DNA
ggcggcggcggcagcggcggcggcggcagcggcggcggcggcagc

SEQ ID NO: 8 - skipped

SEQ ID NO: 9 - protein, 4 aa, synthetic construct
  REGION 1..4: note = Furin substrate
RVRR

SEQ ID NO: 10 - DNA, 12 bp, synthetic construct
  misc_feature 1..12: note = DNA encoding furin substrate
  source 1..12: mol_type = other DNA
cgcgtgcgccgc

SEQ ID NO: 11 - DNA, 42 bp, synthetic construct
  misc_feature 1..42: note = DNA encoding linker comprising furin cleavage site
  source 1..42: mol_type = other DNA
ggcggcggcggcagccgcgtgcgccgcggcggcggcggcagc

SEQ ID NO: 12 - protein, 14 aa, synthetic construct
  REGION 1..14: note = Peptide linker comprising furin cleavage site
GGGGSRVRRGGGGS

SEQ ID NO: 13 - protein, 5 aa, synthetic construct
  REGION 1..5: note = Cathepsin S substrate
PMGLP

SEQ ID NO: 14 - skipped

SEQ ID NO: 15 - DNA, 15 bp, synthetic construct
  misc_feature 1..15: note = DNA encoding cathepsin S substrate
  source 1..15: mol_type = other DNA
ccgatgggcctgccg

SEQ ID NO: 16 - protein, 4 aa, synthetic construct
  REGION 1..4: note = Cathepsin l substrate
HKFR

SEQ ID NO: 17 - DNA, 12 bp, synthetic construct
  misc_feature 1..12: note = DNA encoding cathepsin L substrate
  source 1..12: mol_type = other DNA
cataaatttcgc

SEQ ID NO: 18 - protein, 6 aa, synthetic construct
  REGION 1..6: note = Targeting peptide
RKRGLK

SEQ ID NO: 19 - protein, 9 aa, synthetic construct
  REGION 1..9: note = Targeting peptide
RLYRKRGLK

SEQ ID NO: 20 - protein, 19 aa, synthetic construct
  REGION 1..19: note = Targeting peptide
CKNEKKNKIERNNKLKQPP

SEQ ID NO: 21 - protein, 15 aa, Arachis hypogaea
DNILVIQQGQATVTV

SEQ ID NO: 22 - protein, 15 aa, Arachis hypogaea
GALMLPHFNSKAMVI

SEQ ID NO: 23 - protein, 20 aa, Arachis hypogaea
KAMVIVVVNKGTGNLELVAV

SEQ ID NO: 24 - protein, 15 aa, Arachis hypogaea
LEAAFNAEFNEIRRV

SEQ ID NO: 25 - protein, 15 aa, Arachis hypogaea
NEGVIVKVSKEHVEE

SEQ ID NO: 26 - protein, 15 aa, Arachis hypogaea
QLQDLDMMLTCVEIK

SEQ ID NO: 27 - protein, 15 aa, Arachis hypogaea
QRSRQFQNLQNHRIV

SEQ ID NO: 28 - protein, 15 aa, Arachis hypogaea
QVEKLIKNQKESHFV

SEQ ID NO: 29 - protein, 15 aa, Arachis hypogaea
ELVAVRKEQQQRGRR

SEQ ID NO: 30 - protein, 15 aa, Arachis hypogaea
EVRRYTARLKEGDVF

SEQ ID NO: 31 - protein, 15 aa, Arachis hypogaea
ISYILNRHDNQNLRV

SEQ ID NO: 32 - protein, 15 aa, Arachis hypogaea
KAMVIVVVNKGTGNL

SEQ ID NO: 33 - protein, 15 aa, Arachis hypogaea
LQRFDQRSRQFQNLQ

SEQ ID NO: 34 - protein, 15 aa, Arachis hypogaea
NQNLRVAKISMPVNT

SEQ ID NO: 35 - protein, 15 aa, Arachis hypogaea
RCTKLEYDPRCVYDP

SEQ ID NO: 36 - protein, 15 aa, Arachis hypogaea
RKSFNLDEGHALRIP

SEQ ID NO: 37 - protein, 15 aa, Arachis hypogaea
SNREVRRYTARLKEG

SEQ ID NO: 38 - protein, 15 aa, Arachis hypogaea
SSELHLLGFGINAEN

SEQ ID NO: 39 - protein, 15 aa, Arachis hypogaea
VLASVSATHAKSSPY

SEQ ID NO: 40 - protein, 20 aa, Arachis hypogaea
IFLAGDKDNVIDQIEKQAKD

SEQ ID NO: 41 - protein, 20 aa, Arachis hypogaea
ISYILNRHDNQNLRVAKISM

SEQ ID NO: 42 - protein, 20 aa, Arachis hypogaea
LEAAFNAEFNEIRRVLLEEN

SEQ ID NO: 43 - protein, 20 aa, Arachis hypogaea
MRGRVSPLMLLLGILVLASV

SEQ ID NO: 44 - protein, 20 aa, Arachis hypogaea
NEGVIVKVSKEHVEELTKHA

SEQ ID NO: 45 - protein, 20 aa, Arachis hypogaea
QRSRQFQNLQNHRIVQIEAK

SEQ ID NO: 46 - protein, 20 aa, Arachis hypogaea
RFSTRYGNQNGRIRVLQRFD

SEQ ID NO: 47 - protein, 20 aa, Arachis hypogaea
RTRGRQPGDYDDDRRQPRRE

SEQ ID NO: 48 - protein, 20 aa, Arachis hypogaea
DNILVIQQGQATVTVANGNN

SEQ ID NO: 49 - protein, 20 aa, Arachis hypogaea
EEEGSNREVRRYTARLKEGD

SEQ ID NO: 50 - protein, 20 aa, Arachis hypogaea
FNEIRRVLLEENAGGEQEER

SEQ ID NO: 51 - protein, 20 aa, Arachis hypogaea
KEGDVFIMPAAHPVAINASS

SEQ ID NO: 52 - protein, 20 aa, Arachis hypogaea
LELVAVRKEQQQRGRREEEE

SEQ ID NO: 53 - protein, 20 aa, Arachis hypogaea
LQNHRIVQIEAKPNTLVLPK

SEQ ID NO: 54 - protein, 20 aa, Arachis hypogaea
NNFGKLFEVKPDKKNPQLQD

SEQ ID NO: 55 - protein, 20 aa, Arachis hypogaea
NSKAMVIVVVNKGTGNLELV

SEQ ID NO: 56 - protein, 20 aa, Arachis hypogaea
QKESHFVSARPQSQSQSPSS

SEQ ID NO: 57 - protein, 20 aa, Arachis hypogaea
SKEHVEELTKHAKSVSKKGS

SEQ ID NO: 58 - protein, 20 aa, Arachis hypogaea
TSRNNPFYFPSRRFSTRYGN

SEQ ID NO: 59 - protein, 20 aa, Arachis hypogaea
VVNKGTGNLELVAVRKEQQQ

SEQ ID NO: 60 - protein, 20 aa, Arachis hypogaea
AAHPVAINASSELHLLGFGI

SEQ ID NO: 61 - protein, 20 aa, Arachis hypogaea
AGPREREREEDWRQPREDWR

SEQ ID NO: 62 - protein, 17 aa, Arachis hypogaea
ALMLPHFNSKAMVIVVV

SEQ ID NO: 63 - protein, 20 aa, Arachis hypogaea
ALMLPHFNSKAMVIVVVNKG

SEQ ID NO: 64 - protein, 19 aa, Arachis hypogaea
AMVIVVVNKGTGNLELVAV

SEQ ID NO: 65 - protein, 20 aa, Arachis hypogaea
AVRKEQQQRGRREEEEDEDE

SEQ ID NO: 66 - protein, 20 aa, Arachis hypogaea
CQQEPDDLKQKACESRCTKL

SEQ ID NO: 67 - protein, 20 aa, Arachis hypogaea
DEEEEGSNREVRRYTARLKE

SEQ ID NO: 68 - protein, 20 aa, Arachis hypogaea
DLSNNFGKLFEVKPDKKNPQ

SEQ ID NO: 69 - protein, 20 aa, Arachis hypogaea
EDWRQPREDWRRPSHQQPRK

SEQ ID NO: 70 - protein, 20 aa, Arachis hypogaea
EQVEKLIKNQKESHFVSARP

SEQ ID NO: 71 - protein, 12 aa, Arachis hypogaea
EVKPDKKNPQLQ

SEQ ID NO: 72 - protein, 20 aa, Arachis hypogaea
EVRRYTARLKEGDVFIMPAA

SEQ ID NO: 73 - protein, 20 aa, Arachis hypogaea
FDQRSRQFQNLQNHRIVQIE

SEQ ID NO: 74 - protein, 20 aa, Arachis hypogaea
FEDFFPASSRDQSSYLQGFS

SEQ ID NO: 75 - protein, 20 aa, Arachis hypogaea
FEVKPDKKNPQLQDLDMMLT

SEQ ID NO: 76 - protein, 20 aa, Arachis hypogaea
FNAEFNEIRRVLLEENAGGE

SEQ ID NO: 77 - protein, 9 aa, Arachis hypogaea
FQNLQNHRI

SEQ ID NO: 78 - protein, 20 aa, Arachis hypogaea
FQNLQNHRIVQIEAKPNTLV

SEQ ID NO: 79 - protein, 20 aa, Arachis hypogaea
FSRNTLEAAFNAEFNEIRRV

SEQ ID NO: 80 - protein, 18 aa, Arachis hypogaea
GDVFIMPAAHPVAINASS

SEQ ID NO: 81 - protein, 20 aa, Arachis hypogaea
GEQEERGQRRWSTRSSENNE

SEQ ID NO: 82 - protein, 20 aa, Arachis hypogaea
GEQEWGTPGSHVREETSRNN

SEQ ID NO: 83 - protein, 20 aa, Arachis hypogaea
GINAENNHRIFLAGDKDNVI

SEQ ID NO: 84 - protein, 20 aa, Arachis hypogaea
GRREEEEDEDEEEEGSNREV

SEQ ID NO: 85 - protein, 20 aa, Arachis hypogaea
HAKSVSKKGSEEEGDITNPI

SEQ ID NO: 86 - protein, 20 aa, Arachis hypogaea
HALRIPSGFISYILNRHDNQ

SEQ ID NO: 87 - protein, 20 aa, Arachis hypogaea
IEAKPNTLVLPKHADADNIL

SEQ ID NO: 88 - protein, 20 aa, Arachis hypogaea
ILVIQQGQATVTVANGNNRK

SEQ ID NO: 89 - protein, 11 aa, Arachis hypogaea
KAMVIVVVNKG

SEQ ID NO: 90 - protein, 20 aa, Arachis hypogaea
KDLAFPGSGEQVEKLIKNQK

SEQ ID NO: 91 - protein, 20 aa, Arachis hypogaea
KEHVEELTKHAKSVSKKGSE

SEQ ID NO: 92 - protein, 20 aa, Arachis hypogaea
KGTGNLELVAVRKEQQQRGR

SEQ ID NO: 93 - protein, 20 aa, Arachis hypogaea
KLEYDPRCVYDPRGHTGTTN

SEQ ID NO: 94 - protein, 20 aa, Arachis hypogaea
LLLGILVLASVSATHAKSSP

SEQ ID NO: 95 - protein, 20 aa, Arachis hypogaea
LPKHADADNILVIQQGQATV

SEQ ID NO: 96 - protein, 20 aa, Arachis hypogaea
LTCVEIKEGALMLPHFNSKA

SEQ ID NO: 97 - protein, 20 aa, Arachis hypogaea
NGRIRVLQRFDQRSRQFQNL

SEQ ID NO: 98 - protein, 20 aa, Arachis hypogaea
NLQNHRIVQIEAKPNTLVLP

SEQ ID NO: 99 - protein, 17 aa, Arachis hypogaea
NNFGKLFEVKPDKKNPQ

SEQ ID NO: 100 - protein, 19 aa, Arachis hypogaea
NNFGKLFEVKPDKKNPQLQ

SEQ ID NO: 101 - protein, 20 aa, Arachis hypogaea
NNPFYFPSRRFSTRYGNQNG

SEQ ID NO: 102 - protein, 20 aa, Arachis hypogaea
NQNLRVAKISMPVNTPGQFE

SEQ ID NO: 103 - protein, 20 aa, Arachis hypogaea
PCAQRCLQSCQQEPDDLKQK

SEQ ID NO: 104 - protein, 20 aa, Arachis hypogaea
PINLREGEPDLSNNFGKLFE

SEQ ID NO: 105 - protein, 20 aa, Arachis hypogaea
PQLQDLDMMLTCVEIKEGAL

SEQ ID NO: 106 - protein, 20 aa, Arachis hypogaea
QEEENQGGKGPLLSILKAFN

SEQ ID NO: 107 - protein, 20 aa, Arachis hypogaea
QKACESRCTKLEYDPRCVYD

SEQ ID NO: 108 - protein, 20 aa, Arachis hypogaea
RDQSSYLQGFSRNTLEAAFN

SEQ ID NO: 109 - protein, 20 aa, Arachis hypogaea
REEGGRWGPAGPREREREED

SEQ ID NO: 110 - protein, 13 aa, Arachis hypogaea
RIVQIEAKPNTLV

SEQ ID NO: 111 - protein, 20 aa, Arachis hypogaea
RKSFNLDEGHALRIPSGFIS

SEQ ID NO: 112 - protein, 20 aa, Arachis hypogaea
RVLLEENAGGEQEERGQRRW

SEQ ID NO: 113 - protein, 20 aa, Arachis hypogaea
RWSTRSSENNEGVIVKVSKE

SEQ ID NO: 114 - protein, 20 aa, Arachis hypogaea
SEEEGDITNPINLREGEPDL

SEQ ID NO: 115 - protein, 20 aa, Arachis hypogaea
SHVREETSRNNPFYFPSRRF

SEQ ID NO: 116 - protein, 20 aa, Arachis hypogaea
SMPVNTPGQFEDFFPASSRD

SEQ ID NO: 117 - protein, 20 aa, Arachis hypogaea
SPYQKKTENPCAQRCLQSCQ

SEQ ID NO: 118 - protein, 20 aa, Arachis hypogaea
SSELHLLGFGINAENNHRIF

SEQ ID NO: 119 - protein, 20 aa, Arachis hypogaea
SVSATHAKSSPYQKKTENPC

SEQ ID NO: 120 - protein, 20 aa, Arachis hypogaea
TNQRSPPGERTRGRQPGDYD

SEQ ID NO: 121 - protein, 20 aa, Arachis hypogaea
TVTVANGNNRKSFNLDEGHA

SEQ ID NO: 122 - protein, 17 aa, Arachis hypogaea
VEIKEGALMLPHFNSKA

SEQ ID NO: 123 - protein, 20 aa, Arachis hypogaea
VIDQIEKQAKDLAFPGSGEQ

SEQ ID NO: 124 - protein, 20 aa, Arachis hypogaea
WRRPSHQQPRKIRPEGREGE

SEQ ID NO: 125 - protein, 19 aa, Arachis hypogaea
WSTRSSENNEGVIVKVSKE

SEQ ID NO: 126 - protein, 20 aa, Arachis hypogaea
YDDDRRQPRREEGGRWGPAG

SEQ ID NO: 127 - protein, 20 aa, Arachis hypogaea
YDPRGHTGTTNQRSPPGERT

SEQ ID NO: 128 - protein, 15 aa, Arachis hypogaea
EGSNREVRRYTARLK

SEQ ID NO: 129 - protein, 15 aa, Arachis hypogaea
FGINAENNHRIFLAG

SEQ ID NO: 130 - protein, 15 aa, Arachis hypogaea
GQFEDFFPASSRDQS

SEQ ID NO: 131 - protein, 15 aa, Arachis hypogaea
IDQIEKQAKDLAFPG

SEQ ID NO: 132 - protein, 15 aa, Arachis hypogaea
PHFNSKAMVIVVVNK

SEQ ID NO: 133 - protein, 15 aa, Arachis hypogaea
ATVTVANGNNRKSFN

SEQ ID NO: 134 - protein, 15 aa, Arachis hypogaea
EGDVFIMPAAHPVAI

SEQ ID NO: 135 - protein, 15 aa, Arachis hypogaea
EHVEELTKHAKSVSK

SEQ ID NO: 136 - protein, 15 aa, Arachis hypogaea
ENNHRIFLAGDKDNV

SEQ ID NO: 137 - protein, 15 aa, Arachis hypogaea
EVKPDKKNPQLQDLD

SEQ ID NO: 138 - protein, 15 aa, Arachis hypogaea
FQNLQNHRIVQIEAK

SEQ ID NO: 139 - protein, 15 aa, Arachis hypogaea
GRIRVLQRFDQRSRQ

SEQ ID NO: 140 - protein, 15 aa, Arachis hypogaea
HFVSARPQSQSQSPS

SEQ ID NO: 141 - protein, 15 aa, Arachis hypogaea
HPVAINASSELHLLG

SEQ ID NO: 142 - protein, 15 aa, Arachis hypogaea
HRIVQIEAKPNTLVL

SEQ ID NO: 143 - protein, 15 aa, Arachis hypogaea
MMLTCVEIKEGALML

SEQ ID NO: 144 - protein, 15 aa, Arachis hypogaea
NAEFNEIRRVLLEEN

SEQ ID NO: 145 - protein, 15 aa, Arachis hypogaea
PDLSNNFGKLFEVKP

SEQ ID NO: 146 - protein, 15 aa, Arachis hypogaea
QGGKGPLLSILKAFN

SEQ ID NO: 147 - protein, 15 aa, Arachis hypogaea
RNNPFYFPSRRFSTR

SEQ ID NO: 148 - protein, 15 aa, Arachis hypogaea
SYLQGFSRNTLEAAF

SEQ ID NO: 149 - protein, 15 aa, Arachis hypogaea
DADNILVIQQGQATV

SEQ ID NO: 150 - protein, 15 aa, Arachis hypogaea
FQNLQNHRIVQIEAR

SEQ ID NO: 151 - protein, 15 aa, Arachis hypogaea
GRIRVLQRFDQRSKQ

SEQ ID NO: 152 - protein, 15 aa, Arachis hypogaea
IPSGFISYILNRHDN

SEQ ID NO: 153 - protein, 15 aa, Arachis hypogaea
PHFNSKAMVIVVINK

SEQ ID NO: 154 - protein, 15 aa, Arachis hypogaea
RCTKLEYDPRCVYDT

SEQ ID NO: 155 - protein, 20 aa, Arachis hypogaea
AHASARQQWELQGDRRCQSQ

SEQ ID NO: 156 - protein, 20 aa, Arachis hypogaea
AKLTILVALALFLLAAHASA

SEQ ID NO: 157 - protein, 19 aa, Arachis hypogaea
ALQQIMENQSDRLQGRQQE

SEQ ID NO: 158 - protein, 20 aa, Arachis hypogaea
ANLRPCEQHLMQKIQRDEDS

SEQ ID NO: 159 - protein, 13 aa, Arachis hypogaea
CDLDVESGGRDRY

SEQ ID NO: 160 - protein, 20 aa, Arachis hypogaea
CNELNEFENNQRCMCEALQQ

SEQ ID NO: 161 - protein, 20 aa, Arachis hypogaea
DSYERDPYSPSQDPYSPSPY

SEQ ID NO: 162 - protein, 20 aa, Arachis hypogaea
ELNEFENNQRCMCEALQQIM

SEQ ID NO: 163 - protein, 19 aa, Arachis hypogaea
FENNQRCMCEALQQIMENQ

SEQ ID NO: 164 - protein, 18 aa, Arachis hypogaea
FKRELRNLPQQCGLRAPQ

SEQ ID NO: 165 - protein, 20 aa, Arachis hypogaea
FLLAAHASARQQWELQGDRR

SEQ ID NO: 166 - protein, 20 aa, Arachis hypogaea
GLRAPQRCDLDVESGGRDRY

SEQ ID NO: 167 - protein, 20 aa, Arachis hypogaea
GRQQEQQFKRELRNLPQQCG

SEQ ID NO: 168 - protein, 19 aa, Arachis hypogaea
HLMQKIQRDEDSYERDPYS

SEQ ID NO: 169 - protein, 20 aa, Arachis hypogaea
IMENQSDRLQGRQQEQQFKR

SEQ ID NO: 170 - protein, 20 aa, Arachis hypogaea
KRELRNLPQQCGLRAPQRCD

SEQ ID NO: 171 - protein, 20 aa, Arachis hypogaea
LERANLRPCEQHLMQKIQRD

SEQ ID NO: 172 - protein, 20 aa, Arachis hypogaea
LFLLAAHASARQQWELQGDR

SEQ ID NO: 173 - protein, 20 aa, Arachis hypogaea
LMQKIQRDEDSYERDPYSPS

SEQ ID NO: 174 - protein, 19 aa, Arachis hypogaea
LRAPQRCDLDVESGGRDRY

SEQ ID NO: 175 - protein, 19 aa, Arachis hypogaea
LRNLPQQCGLRAPQRCDLD

SEQ ID NO: 176 - protein, 20 aa, Arachis hypogaea
LRPCEQHLMQKIQRDEDSYE

SEQ ID NO: 177 - protein, 20 aa, Arachis hypogaea
LVALALFLLAAHASARQQWE

SEQ ID NO: 178 - protein, 20 aa, Arachis hypogaea
PQQCGLRAPQRCDLDVESGG

SEQ ID NO: 179 - protein, 20 aa, Arachis hypogaea
PSQDPYSPSPYDRRGAGSSQ

SEQ ID NO: 180 - protein, 20 aa, Arachis hypogaea
PYDRRGAGSSQHQERCCNEL

SEQ ID NO: 181 - protein, 20 aa, Arachis hypogaea
QCGLRAPQRCDLDVESGGRD

SEQ ID NO: 182 - protein, 20 aa, Arachis hypogaea
QGRQQEQQFKRELRNLPQQC

SEQ ID NO: 183 - protein, 19 aa, Arachis hypogaea
RCMCEALQQIMENQSDRLQ

SEQ ID NO: 184 - protein, 20 aa, Arachis hypogaea
RCMCEALQQIMENQSDRLQG

SEQ ID NO: 185 - protein, 20 aa, Arachis hypogaea
RQQWELQGDRRCQSQLERAN

SEQ ID NO: 186 - protein, 20 aa, Arachis hypogaea
RRCQSQLERANLRPCEQHLM

SEQ ID NO: 187 - protein, 20 aa, Arachis hypogaea
SDRLQGRQQEQQFKRELRNL

SEQ ID NO: 188 - protein, 20 aa, Arachis hypogaea
SQHQERCCNELNEFENNQRC

SEQ ID NO: 189 - protein, 11 aa, Arachis hypogaea
ANLRPCEQHLM

SEQ ID NO: 190 - protein, 12 aa, Arachis hypogaea
ELNEFENNQRCM

SEQ ID NO: 191 - protein, 17 aa, Arachis hypogaea
ELNEFENNQRCMCEALQ

SEQ ID NO: 192 - protein, 13 aa, Arachis hypogaea
FENNQRCMCEALQ

SEQ ID NO: 193 - protein, 14 aa, Arachis hypogaea
RELRNLPQQCGLRA

SEQ ID NO: 194 - protein, 13 aa, Arachis hypogaea
SQLERANLRPCEQ

SEQ ID NO: 195 - protein, 16 aa, Arachis hypogaea
SQLERANLRPCEQHLM

SEQ ID NO: 196 - protein, 20 aa, Arachis hypogaea
ALQQIMENQSDRLQGRQQEQ

SEQ ID NO: 197 - protein, 20 aa, Arachis hypogaea
CQSQLERANLRPCEQHLMQK

SEQ ID NO: 198 - protein, 20 aa, Arachis hypogaea
DPYSPSQDPYSPSPYDRRGA

SEQ ID NO: 199 - protein, 20 aa, Arachis hypogaea
DRLQGRQQEQQFKRELRNLP

SEQ ID NO: 200 - protein, 20 aa, Arachis hypogaea
DRRGAGSSQHQERCCNELNE

SEQ ID NO: 201 - protein, 20 aa, Arachis hypogaea
ERANLRPCEQHLMQKIQRDE

SEQ ID NO: 202 - protein, 20 aa, Arachis hypogaea
FENNQRCMCEALQQIMENQS

SEQ ID NO: 203 - protein, 20 aa, Arachis hypogaea
GSSQHQERCCNELNEFENNQ

SEQ ID NO: 204 - protein, 20 aa, Arachis hypogaea
HASARQQWELQGDRRCQSQL

SEQ ID NO: 205 - protein, 20 aa, Arachis hypogaea
HLMQKIQRDEDSYERDPYSP

SEQ ID NO: 206 - protein, 20 aa, Arachis hypogaea
IQRDEDSYERDPYSPSQDPY

SEQ ID NO: 207 - protein, 20 aa, Arachis hypogaea
LRNLPQQCGLRAPQRCDLDV

SEQ ID NO: 208 - protein, 20 aa, Arachis hypogaea
MENQSDRLQGRQQEQQFKRE

SEQ ID NO: 209 - protein, 20 aa, Arachis hypogaea
MLTILVALALFLLAAHASAR

SEQ ID NO: 210 - protein, 20 aa, Arachis hypogaea
NELNEFENNQRCMCEALQQI

SEQ ID NO: 211 - protein, 20 aa, Arachis hypogaea
QERCCNELNEFENNQRCMCE

SEQ ID NO: 212 - protein, 20 aa, Arachis hypogaea
QFKRELRNLPQQCGLRAPQR

SEQ ID NO: 213 - protein, 20 aa, Arachis hypogaea
QGDRRCQSQLERANLRPCEQ

SEQ ID NO: 214 - protein, 20 aa, Arachis hypogaea
QQCGLRAPQRCDLDVESGGR

SEQ ID NO: 215 - protein, 20 aa, Arachis hypogaea
QQWELQGDRRCQSQLERANL

SEQ ID NO: 216 - protein, 18 aa, Arachis hypogaea
RAPQRCDLDVESGGRDRY

SEQ ID NO: 217 - protein, 20 aa, Arachis hypogaea
RPCEQHLMQKIQRDEDSYER

SEQ ID NO: 218 - protein, 20 aa, Arachis hypogaea
RQQEQQFKRELRNLPQQCGL

SEQ ID NO: 219 - protein, 7 aa, Arachis hypogaea
SGGRDRY

SEQ ID NO: 220 - protein, 20 aa, Arachis hypogaea
SPSPYDRRGAGSSQHQERCC

SEQ ID NO: 221 - protein, 20 aa, Arachis hypogaea
SQDPYSPSPYDRRGAGSSQH

SEQ ID NO: 222 - protein, 20 aa, Arachis hypogaea
VALALFLLAAHASARQQWEL

SEQ ID NO: 223 - protein, 21 aa, Arachis hypogaea
CNELNEFENNQRCMCEALQQI

SEQ ID NO: 224 - protein, 15 aa, Arachis hypogaea
ALFLLAAHASARQQW

SEQ ID NO: 225 - protein, 15 aa, Arachis hypogaea
ARQQWELQGDRRCQS

SEQ ID NO: 226 - protein, 15 aa, Arachis hypogaea
VTVRGGLRILSPDRK

SEQ ID NO: 227 - protein, 15 aa, Arachis hypogaea
EYVAFKTDSRPSIAN

SEQ ID NO: 228 - protein, 15 aa, Arachis hypogaea
LAGENSVIDNLPEEV

SEQ ID NO: 229 - protein, 15 aa, Arachis hypogaea
QEFLRYQQQSRQSRR

SEQ ID NO: 230 - protein, 15 aa, Arachis hypogaea
EQGAIVTVRGGLRIL

SEQ ID NO: 231 - protein, 15 aa, Arachis hypogaea
LILRWLGLSAEYGNL

SEQ ID NO: 232 - protein, 15 aa, Arachis hypogaea
QARQLKNNNPFKFFV

SEQ ID NO: 233 - protein, 15 aa, Arachis hypogaea
SGFTPEFLAQAFQVD

SEQ ID NO: 234 - protein, 15 aa, Arachis hypogaea
SIIYALRGRAHVQVV

SEQ ID NO: 235 - protein, 15 aa, Arachis hypogaea
AFQVDDRQIVQNLRG

SEQ ID NO: 236 - protein, 15 aa, Arachis hypogaea
EFLAQAFQVDDRQIV

SEQ ID NO: 237 - protein, 15 aa, Arachis hypogaea
EYLAFKTDSRPSIAN

SEQ ID NO: 238 - protein, 15 aa, Arachis hypogaea
FVPHYTLNAHTIVVA

SEQ ID NO: 239 - protein, 15 aa, Arachis hypogaea
GSRPHYRQISPRVRG

SEQ ID NO: 240 - protein, 15 aa, Arachis hypogaea
HVQVVDSNGNRVYDE

SEQ ID NO: 241 - protein, 15 aa, Arachis hypogaea
NTNAHSIIYALRGRA

SEQ ID NO: 242 - protein, 15 aa, Arachis hypogaea
QAGENSIIDNLPEEV

SEQ ID NO: 243 - protein, 15 aa, Arachis hypogaea
QEFLQHAFQVDRQTV

SEQ ID NO: 244 - protein, 15 aa, Arachis hypogaea
QSDNFEYVAFKTDSR

SEQ ID NO: 245 - protein, 15 aa, Arachis hypogaea
RRPFYSNAPLEIYVQ

SEQ ID NO: 246 - protein, 15 aa, Arachis hypogaea
SGFAQEFLQHAFQVD

SEQ ID NO: 247 - protein, 15 aa, Arachis hypogaea
SRLVLRRNALRRPFY

SEQ ID NO: 248 - protein, 15 aa, Arachis hypogaea
TIVVALNGRAHVQVV

SEQ ID NO: 249 - protein, 15 aa, Arachis hypogaea
TLNAHTIVVALNGRA

SEQ ID NO: 250 - protein, 15 aa, Arachis hypogaea
KSTILVALLALVLVA

SEQ ID NO: 251 - protein, 15 aa, Arachis hypogaea
STILVALLALVLVAH

SEQ ID NO: 252 - protein, 15 aa, Arachis hypogaea
AKSTILVALLALVLV

SEQ ID NO: 253 - protein, 15 aa, Arachis hypogaea
MAKSTILVALLALVL

SEQ ID NO: 254 - protein, 15 aa, Arachis hypogaea
TILVALLALVLVAHA

SEQ ID NO: 255 - protein, 15 aa, Arachis hypogaea
ILVALLALVLVAHAS

SEQ ID NO: 256 - protein, 15 aa, Arachis hypogaea
LVALLALVLVAHASA

SEQ ID NO: 257 - protein, 15 aa, Arachis hypogaea
ALVLVAHASAMRRER

SEQ ID NO: 258 - protein, 15 aa, Arachis hypogaea
KSTILVALLALVLVA

SEQ ID NO: 259 - protein, 15 aa, Arachis hypogaea
LALVLVAHASAMRRE

SEQ ID NO: 260 - protein, 15 aa, Arachis hypogaea
LLALVLVAHASAMRR

SEQ ID NO: 261 - protein, 15 aa, Arachis hypogaea
LVLVAHASAMRRERG

SEQ ID NO: 262 - protein, 15 aa, Arachis hypogaea
AKSTILVALLALVLV

SEQ ID NO: 263 - protein, 15 aa, Arachis hypogaea
ALVLVAHASAMRRER

SEQ ID NO: 264 - protein, 15 aa, Arachis hypogaea
MAKSTILVALLALVL

SEQ ID NO: 265 - protein, 15 aa, Arachis hypogaea
LALVLVAHASAMRRE

SEQ ID NO: 266 - protein, 15 aa, Arachis hypogaea
STILVALLALVLVAH

SEQ ID NO: 267 - protein, 15 aa, Arachis hypogaea
ALLALVLVAHASAMR

SEQ ID NO: 268 - protein, 15 aa, Arachis hypogaea
LVLVAHASAMRRERG

SEQ ID NO: 269 - protein, 15 aa, Arachis hypogaea
LLALVLVAHASAMRR

SEQ ID NO: 270 - protein, 15 aa, Arachis hypogaea
TILVALLALVLVAHA

SEQ ID NO: 271 - protein, 15 aa, Arachis hypogaea
VLVAHASAMRRERGR

SEQ ID NO: 272 - protein, 15 aa, Arachis hypogaea
TILVALLALVLVAHA

SEQ ID NO: 273 - protein, 15 aa, Arachis hypogaea
VALLALVLVAHASAM

SEQ ID NO: 274 - protein, 15 aa, Arachis hypogaea
ILVALLALVLVAHAS

SEQ ID NO: 275 - protein, 15 aa, Arachis hypogaea
LLALVLVAHASAMRR

SEQ ID NO: 276 - protein, 15 aa, Arachis hypogaea
LVALLALVLVAHASA

SEQ ID NO: 277 - protein, 15 aa, Arachis hypogaea
LVLVAHASAMRRERG

SEQ ID NO: 278 - protein, 15 aa, Arachis hypogaea
LALVLVAHASAMRRE

SEQ ID NO: 279 - protein, 15 aa, Arachis hypogaea
VALLALVLVAHASAM

SEQ ID NO: 280 - protein, 15 aa, Arachis hypogaea
ALVLVAHASAMRRER

SEQ ID NO: 281 - protein, 15 aa, Arachis hypogaea
LALVLVAHASAMRRE

SEQ ID NO: 282 - protein, 15 aa, Arachis hypogaea
KSTILVALLALVLVA

SEQ ID NO: 283 - protein, 15 aa, Arachis hypogaea
LLALVLVAHASAMRR

SEQ ID NO: 284 - protein, 15 aa, Arachis hypogaea
AKSTILVALLALVLV

SEQ ID NO: 285 - protein, 15 aa, Arachis hypogaea
AKSTILVALLALVLV

SEQ ID NO: 286 - protein, 15 aa, Arachis hypogaea
ILVALLALVLVAHAS

SEQ ID NO: 287 - protein, 15 aa, Arachis hypogaea
KSTILVALLALVLVA

SEQ ID NO: 288 - protein, 15 aa, Arachis hypogaea
LVALLALVLVAHASA

SEQ ID NO: 289 - protein, 15 aa, Arachis hypogaea
MAKSTILVALLALVL

SEQ ID NO: 290 - protein, 15 aa, Arachis hypogaea
STILVALLALVLVAH

SEQ ID NO: 291 - protein, 15 aa, Arachis hypogaea
TILVALLALVLVAHA

SEQ ID NO: 292 - protein, 15 aa, Arachis hypogaea
VLVAHASAMRRERGR

SEQ ID NO: 293 - protein, 15 aa, Arachis hypogaea
LVLVAHASAMRRERG

SEQ ID NO: 294 - protein, 15 aa, Arachis hypogaea
STILVALLALVLVAH

SEQ ID NO: 295 - protein, 15 aa, Arachis hypogaea
VALLALVLVAHASAM

SEQ ID NO: 296 - protein, 15 aa, Arachis hypogaea
ILVALLALVLVAHAS

SEQ ID NO: 297 - protein, 15 aa, Arachis hypogaea
MAKSTILVALLALVL

SEQ ID NO: 298 - protein, 15 aa, Arachis hypogaea
ALVLVAHASAMRRER

SEQ ID NO: 299 - protein, 15 aa, Arachis hypogaea
ALLALVLVAHASAMR

SEQ ID NO: 300 - protein, 15 aa, Arachis hypogaea
LLALVLVAHASAMRR

SEQ ID NO: 301 - protein, 15 aa, Arachis hypogaea
ALLALVLVAHASAMR

SEQ ID NO: 302 - protein, 15 aa, Arachis hypogaea
MAKSTILVALLALVL

SEQ ID NO: 303 - protein, 15 aa, Arachis hypogaea
VLVAHASAMRRERGR

SEQ ID NO: 304 - protein, 15 aa, Arachis hypogaea
AKSTILVALLALVLV

SEQ ID NO: 305 - protein, 15 aa, Arachis hypogaea
KSTILVALLALVLVA

SEQ ID NO: 306 - protein, 15 aa, Arachis hypogaea
LALVLVAHASAMRRE

SEQ ID NO: 307 - protein, 15 aa, Arachis hypogaea
LVALLALVLVAHASA

SEQ ID NO: 308 - protein, 15 aa, Arachis hypogaea
MAKSTILVALLALVL

SEQ ID NO: 309 - protein, 15 aa, Arachis hypogaea
STILVALLALVLVAH

SEQ ID NO: 310 - protein, 15 aa, Arachis hypogaea
AKSTILVALLALVLV

SEQ ID NO: 311 - protein, 15 aa, Arachis hypogaea
KSTILVALLALVLVA

SEQ ID NO: 312 - protein, 15 aa, Arachis hypogaea
STILVALLALVLVAH

SEQ ID NO: 313 - protein, 15 aa, Arachis hypogaea
TILVALLALVLVAHA

SEQ ID NO: 314 - protein, 15 aa, Arachis hypogaea
TILVALLALVLVAHA

SEQ ID NO: 315 - protein, 15 aa, Arachis hypogaea
LVAHASAMRRERGRQ

SEQ ID NO: 316 - protein, 15 aa, Arachis hypogaea
LVAHASAMRRERGRQ

SEQ ID NO: 317 - protein, 15 aa, Arachis hypogaea
KSTILVALLALVLVA

SEQ ID NO: 318 - protein, 15 aa, Arachis hypogaea
AKSTILVALLALVLV

SEQ ID NO: 319 - protein, 15 aa, Arachis hypogaea
MAKSTILVALLALVL

SEQ ID NO: 320 - protein, 15 aa, Arachis hypogaea
ALLALVLVAHASAMR

SEQ ID NO: 321 - protein, 15 aa, Arachis hypogaea
LALVLVAHASAMRRE

SEQ ID NO: 322 - protein, 15 aa, Arachis hypogaea
STILVALLALVLVAH

SEQ ID NO: 323 - protein, 15 aa, Arachis hypogaea
LLALVLVAHASAMRR

SEQ ID NO: 324 - protein, 15 aa, Arachis hypogaea
ILVALLALVLVAHAS

SEQ ID NO: 325 - protein, 15 aa, Arachis hypogaea
LALVLVAHASAMRRE

SEQ ID NO: 326 - protein, 20 aa, Arachis hypogaea
FQNLQNHRIVQIEAKPNTLV

SEQ ID NO: 327 - protein, 19 aa, Arachis hypogaea
WSTRSSENNEGVIVKVSKE

SEQ ID NO: 328 - protein, 10 aa, Arachis hypogaea
NEGVIVKVSK

SEQ ID NO: 329 - protein, 19 aa, Arachis hypogaea
NNFGKLFEVKPDKKNPQLQ

SEQ ID NO: 330 - protein, 17 aa, Arachis hypogaea
VEIKEGALMLPHFNSKA

SEQ ID NO: 331 - protein, 6 aa, Arachis hypogaea
EGALML

SEQ ID NO: 332 - protein, 17 aa, Arachis hypogaea
ALMLPHFNSKAMVIVVV

SEQ ID NO: 333 - protein, 11 aa, Arachis hypogaea
PHFNSKAMVIV

SEQ ID NO: 334 - protein, 20 aa, Arachis hypogaea
KAMVIVVVNKGTGNLELVAV

SEQ ID NO: 335 - protein, 18 aa, Arachis hypogaea
GDVFIMPAAHPVAINASS

SEQ ID NO: 336 - protein, 7 aa, Arachis hypogaea
IMPAAHP

SEQ ID NO: 337 - protein, 20 aa, Arachis hypogaea
RRCQSQLFRANLRPCEQHLM

SEQ ID NO: 338 - protein, 9 aa, Arachis hypogaea
LFRANLRPC

SEQ ID NO: 339 - protein, 20 aa, Arachis hypogaea
ELNEFENNQRCMCEALQQIM

SEQ ID NO: 340 - protein, 12 aa, Arachis hypogaea
ELNEFENNQRCM

SEQ ID NO: 341 - protein, 20 aa, Arachis hypogaea
KRFLRNLPQQCGLRAPQRCD

SEQ ID NO: 342 - protein, 12 aa, Arachis hypogaea
RFLRNLPQQCGL

SEQ ID NO: 343 - protein, 20 aa, Arachis hypogaea
RRCQSQLERANLRPCEQHLM

SEQ ID NO: 344 - protein, 10 aa, Arachis hypogaea
NLRPCEQHLM

SEQ ID NO: 345 - protein, 20 aa, Arachis hypogaea
ELNEFENNQRCMCEALQQIM

SEQ ID NO: 346 - protein, 10 aa, Arachis hypogaea
ENNQRCMCEA

SEQ ID NO: 347 - protein, 15 aa, Arachis hypogaea
VLASVSATHAKSSPY

SEQ ID NO: 348 - protein, 15 aa, Arachis hypogaea
RCTKLEYDPRCVYDP

SEQ ID NO: 349 - protein, 15 aa, Arachis hypogaea
RNNPFYFPSRRFSTR

SEQ ID NO: 350 - protein, 15 aa, Arachis hypogaea
GRIRVLQRFDQRSRQ

SEQ ID NO: 351 - protein, 15 aa, Arachis hypogaea
LQRFDQRSRQFQNLQ

SEQ ID NO: 352 - protein, 15 aa, Arachis hypogaea
QRSRQFQNLQNHRIV

SEQ ID NO: 353 - protein, 15 aa, Arachis hypogaea
FQNLQNHRIVQIEAK

SEQ ID NO: 354 - protein, 15 aa, Arachis hypogaea
HRIVQIEAKPNTLVL

SEQ ID NO: 355 - protein, 15 aa, Arachis hypogaea
DNILVIQQGQATVTV

SEQ ID NO: 356 - protein, 15 aa, Arachis hypogaea
ATVTVANGNNRKSFN

SEQ ID NO: 357 - protein, 15 aa, Arachis hypogaea
RKSFNLDEGHALRIP

SEQ ID NO: 358 - protein, 15 aa, Arachis hypogaea
ISYILNRHDNQNLRV

SEQ ID NO: 359 - protein, 15 aa, Arachis hypogaea
NQNLRVAKISMPVNT

SEQ ID NO: 360 - protein, 15 aa, Arachis hypogaea
GQFEDFFPASSRDQS

SEQ ID NO: 361 - protein, 15 aa, Arachis hypogaea
SYLQGFSRNTLEAAF

SEQ ID NO: 362 - protein, 15 aa, Arachis hypogaea
LEAAFNAEFNEIRRV

SEQ ID NO: 363 - protein, 15 aa, Arachis hypogaea
NAEFNEIRRVLLEEN

SEQ ID NO: 364 - protein, 15 aa, Arachis hypogaea
NEGVIVKVSKEHVEE

SEQ ID NO: 365 - protein, 15 aa, Arachis hypogaea
EHVEELTKHAKSVSK

SEQ ID NO: 366 - protein, 15 aa, Arachis hypogaea
PDLSNNFGKLFEVKP

SEQ ID NO: 367 - protein, 15 aa, Arachis hypogaea
EVKPDKKNPQLQDLD

SEQ ID NO: 368 - protein, 15 aa, Arachis hypogaea
QLQDLDMMLTCVEIK

SEQ ID NO: 369 - protein, 15 aa, Arachis hypogaea
MMLTCVEIKEGALML

SEQ ID NO: 370 - protein, 15 aa, Arachis hypogaea
GALMLPHFNSKAMVI

SEQ ID NO: 371 - protein, 15 aa, Arachis hypogaea
KAMVIVVVNKGTGNL

SEQ ID NO: 372 - protein, 15 aa, Arachis hypogaea
ELVAVRKEQQQRGRR

SEQ ID NO: 373 - protein, 15 aa, Arachis hypogaea
EGSNREVRRYTARLK

SEQ ID NO: 374 - protein, 15 aa, Arachis hypogaea
EVRRYTARLKEGDVF

SEQ ID NO: 375 - protein, 15 aa, Arachis hypogaea
EGDVFIMPAAHPVAI

SEQ ID NO: 376 - protein, 15 aa, Arachis hypogaea
HPVAINASSELHLLG

SEQ ID NO: 377 - protein, 15 aa, Arachis hypogaea
FGINAENNHRIFLAG

SEQ ID NO: 378 - protein, 15 aa, Arachis hypogaea
ENNHRIFLAGDKDNV

SEQ ID NO: 379 - protein, 15 aa, Arachis hypogaea
IDQIEKQAKDLAFPG

SEQ ID NO: 380 - protein, 15 aa, Arachis hypogaea
QVEKLIKNQKESHFV

SEQ ID NO: 381 - protein, 15 aa, Arachis hypogaea
HFVSARPQSQSQSPS

SEQ ID NO: 382 - protein, 15 aa, Arachis hypogaea
QGGKGPLLSILKAFN

SEQ ID NO: 383 - protein, 10 aa, Arachis hypogaea
FQNLQNHRIV

SEQ ID NO: 384 - protein, 13 aa, Arachis hypogaea
RIVQIEAKPNTLV

SEQ ID NO: 385 - protein, 20 aa, Arachis hypogaea
FQNLQNHRIVQIEAKPNTLV

SEQ ID NO: 386 - protein, 19 aa, Arachis hypogaea
WSTRSSENNEGVIVKVSKE

SEQ ID NO: 387 - protein, 19 aa, Arachis hypogaea
NNFGKLFEVKPDKKNPQLQ

SEQ ID NO: 388 - protein, 17 aa, Arachis hypogaea
VEIKEGALMLPHFNSKA

SEQ ID NO: 389 - protein, 17 aa, Arachis hypogaea
ALMLPHFNSKAMVIVVV

SEQ ID NO: 390 - protein, 20 aa, Arachis hypogaea
KAMVIVVVNKGTGNLELVAV

SEQ ID NO: 391 - protein, 18 aa, Arachis hypogaea
GDVFIMPAAHPVAINASS

SEQ ID NO: 392 - protein, 12 aa, Arachis hypogaea
ALFLLAAHASAR

SEQ ID NO: 393 - protein, 16 aa, Arachis hypogaea
SQLERANLRPCEQHLM

SEQ ID NO: 394 - protein, 17 aa, Arachis hypogaea
ELNEFENNQRCMCEALQ

SEQ ID NO: 395 - protein, 17 aa, Arachis hypogaea
RCMCEALQQIMENQSDR

SEQ ID NO: 396 - protein, 14 aa, Arachis hypogaea
RELRNLPQQCGLRA

SEQ ID NO: 397 - protein, 13 aa, Arachis hypogaea
QFKRELRNLPQQC

SEQ ID NO: 398 - protein, 20 aa, Arachis hypogaea
FQNLQNHRIVQIEAKPNTLV

SEQ ID NO: 399 - DNA, 60 bp, Arachis hypogaea
  source 1..60: mol_type = other DNA
tttcagaacctgcagaaccatcgcattgtgcagattgaagcgaaaccgaacaccctggtg

SEQ ID NO: 400 - protein, 19 aa, Arachis hypogaea
WSTRSSENNEGVIVKVSKE

SEQ ID NO: 401 - DNA, 57 bp, Arachis hypogaea
  source 1..57: mol_type = other DNA
tggagcacccgcagcagcgaaaacaacgaaggcgtgattgtgaaagtgagcaaagaa

SEQ ID NO: 402 - protein, 19 aa, Arachis hypogaea
NNFGKLFEVKPDKKNPQLQ

SEQ ID NO: 403 - DNA, 57 bp, Arachis hypogaea
  source 1..57: mol_type = other DNA
aacaactttggcaaactgtttgaagtgaaaccggataaaaaaaacccgcagctgcag

SEQ ID NO: 404 - protein, 17 aa, Arachis hypogaea
VEIKEGALMLPHFNSKA

SEQ ID NO: 405 - DNA, 51 bp, Arachis hypogaea
  source 1..51: mol_type = other DNA
gtggaaattaaagaaggcgcgctgatgctgccgcattttaacagcaaagcg

SEQ ID NO: 406 - protein, 17 aa, Arachis hypogaea
ALMLPHFNSKAMVIVVV

SEQ ID NO: 407 - DNA, 51 bp, Arachis hypogaea
  source 1..51: mol_type = other DNA
gcgctgatgctgccgcattttaacagcaaagcgatggtgattgtggtggtg

SEQ ID NO: 408 - protein, 20 aa, Arachis hypogaea
KAMVIVVVNKGTGNLELVAV

SEQ ID NO: 409 - DNA, 60 bp, Arachis hypogaea
  source 1..60: mol_type = other DNA
aaagcgatggtgattgtggtggtgaacaaaggcaccggcaacctggaactggtggcggtg

SEQ ID NO: 410 - protein, 18 aa, Arachis hypogaea
GDVFIMPAAHPVAINASS

SEQ ID NO: 411 - DNA, 54 bp, Arachis hypogaea
  source 1..54: mol_type = other DNA
ggcgatgtgtttattatgccggcggcgcatccggtggcgattaacgcgagcagc

SEQ ID NO: 412 - protein, 16 aa, Arachis hypogaea
SQLERANLRPSEQHLM

SEQ ID NO: 413 - DNA, 48 bp, Arachis hypogaea
  source 1..48: mol_type = other DNA
agccagctggaacgcgcgaacctgcgcccgagcgaacagcatctgatg

SEQ ID NO: 414 - protein, 17 aa, Arachis hypogaea
ELNEFENNQRSMSEALQ

SEQ ID NO: 415 - DNA, 51 bp, Arachis hypogaea
  source 1..51: mol_type = other DNA
gaactgaacgaatttgaaaacaaccagcgcagcatgagcgaagcgctgcag

SEQ ID NO: 416 - protein, 14 aa, Arachis hypogaea
RELRNLPQQSGLRA

SEQ ID NO: 417 - DNA, 42 bp, Arachis hypogaea
  source 1..42: mol_type = other DNA
cgcgaactgcgcaacctgccgcagcagagcggcctgcgcgcg

SEQ ID NO: 418 - protein, 5 aa, synthetic construct
  REGION 1..5: note = Peptide linker
GGGGS

SEQ ID NO: 419 - DNA, 45 bp, synthetic construct; identical to SEQ ID NO: 7
  misc_feature 1..45: note = DNA encoding peptide linker
  source 1..45: mol_type = other DNA

SEQ ID NO: 420 - protein, 5 aa, synthetic construct
  REGION 1..5: note = Peptide linker
GGGGS

SEQ ID NO: 421 - DNA, 933 bp, synthetic construct
  misc_feature 1..933: note = DNA encoding 10 human Arah1 and h2 epitopes separated by flexible linkers
  source 1..933: mol_type = other DNA
tttcagaacctgcagaaccatcgcattgtgcagattgaagcgaaaccgaacaccctggtgggcggcggcggcagcggcggcggcggcagcggcggcggcggcagctggagcacccgcagcagcgaaaacaacgaaggcgtgattgtgaaagtgagcaaagaaggcggcggcggcagcggcggcggcggcagcggcggcggcggcagcaacaactttggcaaactgtttgaagtgaaaccggataaaaaaaacccgcagctgcagggcggcggcggcagcggcggcggcagcggcggcggcggcagcgtggaaattaaagaaggcgcgctgatgctgccgcattttaacagcaaagcgggcggcggcggcagcggcggcggcggcagcggcggcggcggcagcgcgctgatgctgccgcattttaacagcaaagcgatggtgattgtggtggtgggcggcggcggcagcggcggcggcggcagcggcggcggcggcagcaaagcgatggtgattgtggtggtgaacaaaggcaccggcaacctggaactggtggcggtgggcggcggcggcagcggcggcgggcggcagcggcggcggcggcagcggcgatgtgtttattatgccggcggcgcatccggtggcgattaacgcgagcagcggcggcggcggcagcggcggcggcggcagcggcggcggcggcagcagccagctggaacgcgcgaacctgcgcccgagcgaacagcatctgatgggcggcggcggcagcggcggcggcggcagcggcggcggcggcagcgaactgaacgaatttgaaaacaaccagcgcagcatgagcgaagcgctgcagggcggcggcggcagcggcggcggcggcagcggcggcggcggcagccgcgaatgcgcaacctgccgcagcagagcggcctgcgcgcg

SEQ ID NO: 422 - skipped

SEQ ID NO: 423 - protein, 4 aa, synthetic construct
  REGION 1..4: note = Furin substrate
RVRR

SEQ ID NO: 424 - DNA, 12 bp, synthetic construct
  misc_feature 1..12: note = DNA encoding furin substrate
  source 1..12: mol_type = other DNA
cgcgtgcgccgc

SEQ ID NO: 425 - DNA, 42 bp, synthetic construct; identical to SEQ ID NO: 11
  misc_feature 1..42: note = DNA encoding peptide linker comprising furin cleavage site
  source 1..42: mol_type = other DNA

SEQ ID NO: 426 - protein, 14 aa, synthetic construct
  REGION 1..14: note = Peptide linker comprising furin cleavage site
GGGGSRVRRGGGGS

SEQ ID NO: 427 - protein, 4 aa, synthetic construct
  REGION 1..4: note = Furin substrate
RVRR

SEQ ID NO: 428 - DNA, 12 bp, synthetic construct
  misc_feature 1..12: note = DNA encoding furin substrate
  source 1..12: mol_type = other DNA
cgcgtgcgccgc

SEQ ID NO: 429 - DNA, 921 bp, synthetic construct
  misc_feature 1..921: note = DNA encoding 10 human Arah1 and h2 epitopes separated by flexible linkers containing furin cleavage sites
  source 1..921: mol_type = other DNA
tttcagaacctgcagaaccatcgcattgtgcagattgaagcgaaaccgaacaccctggtgggcggcggcggcagccgcgtgcgccgcggcggcggcggcagctggagcacccgcagcagcgaaaacaacgaaggcgtgattgtgaaagtgagcaaagaaggcggcggcggcagccgcgtgcgccgcggcggcggcggcagcaacaactttggcaaactgtttgaagtgaaaccggataaaaaaaacccgcagctgcagggcggcggcggcagccgcgtgcgccgcggcggcggcggcagcgtggaaattaaagaaggcgcgctgatgctgccgcattttaacagcaaagcgggcggcggcggcagccgcgtgcgccgcggcggcggcggcagcgcgctgatgctgccgcattttaacagcaaagcgatggtgattgtggtggtgggcggcggcggcagccgcgtgcgccgcggcggcggcggcagcaaagcgatggtgattgtggtggtgaacaaaggcaccggcaacctggaactggtggcggtgggcggcggcggcagccgcgtgcgccgcggcggcggcggcagcggcgatgtgtttattatgccggcggcgcatccggtggcgattaacgcgagcagcggcggcggcggcagccgcgtgcgccgcggcggcggcggcagcagccagctggaacgcgcgaacctgcgcccgagcgaacagcatctgatgggcggcggcggcagccgcgtgcgccgcggcggcggcggcagcgaactgaacgaatttgaaaacaaccagcgcagcatgagcgaagcgctgcagggcggcggcggcagccgcgtgcgccgcggcggcggcggcagccgcgaactgcgcaacctgccgcagcagagcggcctgcgcgcgcgcgtgcgccgc

SEQ ID NO: 430 - protein, 5 aa, synthetic construct
  REGION 1..5: note = Cathepsin S substrate
PMGLP

SEQ ID NO: 431 - DNA, 15 bp, synthetic construct
  misc_feature 1..15: note = DNA encoding cathepsin S substrate
  source 1..15: mol_type = other DNA
ccgatgggcctgccg

SEQ ID NO: 432 - DNA, 12 bp, synthetic construct
  misc_feature 1..12: note = DNA encoding cathepsin L substrate
  source 1..12: mol_type = other DNA
cataaatttcgc

SEQ ID NO: 433 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 434 - protein, 118 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPA

SEQ ID NO: 435 - DNA, 354 bp, Homo sapiens; identical to SEQ ID NO: 2
  source 1..354: mol_type = other DNA

SEQ ID NO: 436 - DNA, 1320 bp, synthetic construct
  misc_feature 1..1320: note = Codon optimized DNA encoding multi-epitope Ara h nucleic acid sequence
  source 1..1320: mol_type = other DNA
atgatggaccaggccagaagcgccttcagcaacctgttcggcggcgagccactgagctacaccagattcagcctggccagacaggtggacggcgataattctcacgtggaaatgaaactggccgtcgacgaggaagaaaatgccgacaacaataccaaggccaatgtgacaaagcctaagcggtgcagcggcagcatctgttacggcaccatcgccgtgatcgtgttctttctgatcggcttcatgatcggctatctgggctactgcaagggcgtggaacctaagaccgagtgcgagcggctggctggcacagagagccctgtgcgggaagagcccggcgaagatttcccagccggcggaggcgggtctagagtgcggagaggaggcggaggcagcttccagaacctgcaaaaccacagaatcgtgcagatcgaggccaagcccaacaccctggtcggaggcggaggctctagagtgagacgcggcggaggcggaagctggtccaccagaagctctgagaacaatgagggcgttattgtgaaagtgtccaaggaaggcggcggaggctctagagtgagaagaggcggcggcggctccaacaacttcggcaagctgtttgaggtgaagcctgataagaagaacccccagctgcaaggaggcggaggcagccgggtgcggaggggaggcggaggatccgtggaaatcaaggaaggcgccctgatgctgccccactttaacagcaaagccggcggcggcggcagcagagtgcgtcggggtggcggaggcagtgctctgatgctgcctcacttcaacagcaaggccatggtcatcgtggtggtgggcggagggggcagcagagttagaagaggcggcggcggctctaaagccatggtaatcgtggtcgtgaacaagggaacaggcaacctcgagctggtggctgtgggcggtggcggctcccgggtgcggagaggcggaggcggctctggcgacgtgttcatcatgcctgccgctcatcctgtggccatcaacgctagcagcggcggcggcggcagcagggtgagacggggcggcggcgggagcagccagctggaaagagccaatctgagacctagcgagcagcacctgatgggcggcggaggctccagagtgcggcggggtggcggcggcagcgagctgaacgagttcgagaacaaccagcggagcatgagcgaggccctgcagggcggcggaggctctcgcgtgcggagaggaggcggcggcagcagagagctgagaaacctgcctcagcagagcggcctgcgggccagggtgcggagatga

SEQ ID NO: 437 - protein, 14 aa, Homo sapiens
DLMMIEEYPYVVIL

SEQ ID NO: 438 - protein, 19 aa, Homo sapiens
DLRQMRTVTPIRMQGGCGS

SEQ ID NO: 439 - protein, 19 aa, Homo sapiens
FRHYDGRTIIQRDNGYQPN

SEQ ID NO: 440 - protein, 19 aa, Homo sapiens
GCGSCWAFSGVAATESAYL

SEQ ID NO: 441 - protein, 19 aa, Homo sapiens
HNGVVQESYYRYVAREQSC

SEQ ID NO: 442 - protein, 19 aa, Homo sapiens
IEYIQHNGVVQESYYRYVA

SEQ ID NO: 443 - protein, 19 aa, Homo sapiens
IRMQGGCGSCWAFSGVAAT

SEQ ID NO: 444 - protein, 37 aa, Homo sapiens
KNRFLMSAEAFEHLKTQFDLNAETNACSINGNAPAEI

SEQ ID NO: 445 - protein, 13 aa, Homo sapiens
NAQRFGISNYCQI

SEQ ID NO: 446 - protein, 19 aa, Homo sapiens
NKIREALAQTHSAIAVIIG

SEQ ID NO: 447 - protein, 19 aa, Homo sapiens
QESYYRYVAREQSCRRPNA

SEQ ID NO: 448 - protein, 26 aa, Homo sapiens
QIYPPNANKIREALAQTHSAIAHYWT

SEQ ID NO: 449 - protein, 19 aa, Homo sapiens
QSCRRPNAQRFGISNYCQI

SEQ ID NO: 450 - protein, 11 aa, Homo sapiens
RFGISNYCQIY

SEQ ID NO: 451 - protein, 22 aa, Homo sapiens
RFGISNYCQIYPPNANKIREAL

SEQ ID NO: 452 - protein, 19 aa, Homo sapiens
RFGISNYCQIYPPNVNKIR

SEQ ID NO: 453 - protein, 19 aa, Homo sapiens
RPNAQRFGISNYCQIYPPN

SEQ ID NO: 454 - protein, 23 aa, Homo sapiens
SAYLAHRNQSLDLAEQELVDCAS

SEQ ID NO: 455 - protein, 19 aa, Homo sapiens
SAYLAYRNQSLDLAEQELV

SEQ ID NO: 456 - protein, 19 aa, Homo sapiens
TIPRGIEYIQHNGVVQESY

SEQ ID NO: 457 - protein, 18 aa, Homo sapiens
TNACSINGNAPAEIDLRQ

SEQ ID NO: 458 - protein, 19 aa, Homo sapiens
VIIGIKDLDAFRHYDGRTI

SEQ ID NO: 459 - protein, 35 aa, Homo sapiens
VRNSWDTNWGDNGYGYFAANIDLMMIEEYPYVVIL

SEQ ID NO: 460 - protein, 11 aa, Homo sapiens
YAYVAREQSCR

SEQ ID NO: 461 - protein, 19 aa, Homo sapiens
YPPNVNKIREALAQTHSAI

SEQ ID NO: 462 - protein, 19 aa, Homo sapiens
YQPNYHAVNIVGYSNAQGV

SEQ ID NO: 463 - protein, 29 aa, Homo sapiens
VQESYYRYVAREQSCRRPNAQRFGISNYC

SEQ ID NO: 464 - protein, 19 aa, Homo sapiens
ALAQTHTAIAVIIGIKDLD

SEQ ID NO: 465 - protein, 19 aa, Homo sapiens
RFGISNYCQIYPPNANKIR

SEQ ID NO: 466 - protein, 15 aa, Homo sapiens
AVNIVGYSNAQGVDY

SEQ ID NO: 467 - protein, 15 aa, Homo sapiens
LRQMRTVTPIRMQGG

SEQ ID NO: 468 - protein, 15 aa, Homo sapiens
RFGISNYCQIYPPNV

SEQ ID NO: 469 - protein, 15 aa, Homo sapiens
ARKNFLESVKYVQSN

SEQ ID NO: 470 - protein, 15 aa, Homo sapiens
DNGYGYFAANIDLMM

SEQ ID NO: 471 - protein, 15 aa, Homo sapiens
EIDLRQMRTVTPIRM

SEQ ID NO: 472 - protein, 15 aa, Homo sapiens
GGAINHLSDLSLDEF

SEQ ID NO: 473 - protein, 15 aa, Homo sapiens
KAFNKSYATFEDEEA

SEQ ID NO: 474 - protein, 15 aa, Homo sapiens
LALSAVYARPSSIKT

SEQ ID NO: 475 - protein, 15 aa, Homo sapiens
LESVKYVQSNGGAIN

SEQ ID NO: 476 - protein, 15 aa, Homo sapiens
QTHSAIAVIIGIKDL

SEQ ID NO: 477 - protein, 15 aa, Homo sapiens
REALAQTHSAIAVII

SEQ ID NO: 478 - protein, 15 aa, Homo sapiens
RGIEYIQHNGVVQES

SEQ ID NO: 479 - protein, 15 aa, Homo sapiens
SCWAFSGVAATESAY

SEQ ID NO: 480 - protein, 15 aa, Homo sapiens
SGVAATESAYLAYRN

SEQ ID NO: 481 - protein, 33 aa, Homo sapiens
GRTIIQRDNGYQPNYHAVNIVGYSNAQGVDYWI

SEQ ID NO: 482 - protein, 31 aa, Homo sapiens
HNGVVQESYYRYVAREQSCRRPNAQRFGISN

SEQ ID NO: 483 - protein, 31 aa, Homo sapiens
KDLDAFRHYDGRTIIQRDNGYQPNYHAVNIV

SEQ ID NO: 484 - protein, 37 aa, Homo sapiens
REQSCRRPNAQRFGISNYCQIYPPNVNKIREALAQTH

SEQ ID NO: 485 - protein, 34 aa, Homo sapiens
VGYSNAQGVDYWIVRNSWDTNWGDNGYGYFAANI

SEQ ID NO: 486 - protein, 43 aa, Homo sapiens
ATESAYLAYRNQSLDLAEQELVDCASQHGCHGDTIPRGIEYIQ

SEQ ID NO: 487 - protein, 41 aa, Homo sapiens
TNACSINGNAPAEIDLRQMRTVTPIRMQGGCGSCWAFSGVA

SEQ ID NO: 488 - protein, 35 aa, Homo sapiens
VRNSWDTNWGDNGYGYFAANIDIMMIEEYPYVVIL

SEQ ID NO: 489 - protein, 14 aa, Homo sapiens
ASIDGLGVDVPGID

SEQ ID NO: 490 - protein, 15 aa, Homo sapiens
CHGSEPCIIHRGKPF

SEQ ID NO: 491 - protein, 27 aa, Homo sapiens
CPLVKGQQYDIKYTWNVPKIAPKSENV

SEQ ID NO: 492 - protein, 26 aa, Homo sapiens
DIKYTWNVPKIAPKSENVVVTVKVMG

SEQ ID NO: 493 - protein, 15 aa, Homo sapiens
DQVDVKDCANHEIKK

SEQ ID NO: 494 - protein, 20 aa, Homo sapiens
DQVDVKDCANHEIKKVLVPG

SEQ ID NO: 495 - protein, 14 aa, Homo sapiens
EAVFEANQNTKTAK

SEQ ID NO: 496 - protein, 15 aa, Homo sapiens
FEAVQNTKTAKIEIK

SEQ ID NO: 497 - protein, 14 aa, Homo sapiens
GCHGSEPCIIHRGK

SEQ ID NO: 498 - protein, 14 aa, Homo sapiens
HEIKKVLVPGCHGS

SEQ ID NO: 499 - protein, 15 aa, Homo sapiens
HEIKKVLVPGCHGSE

SEQ ID NO: 500 - protein, 19 aa, Homo sapiens
HGSEPCIIHRGKPFQLEAV

SEQ ID NO: 501 - protein, 29 aa, Homo sapiens
HGSEPCIIHRGKPFQLEAVFEANQNTKTA

SEQ ID NO: 502 - protein, 16 aa, Homo sapiens
IDGLEVDVPGIDPNAC

SEQ ID NO: 503 - protein, 14 aa, Homo sapiens
IHRGKPFQLEAVFE

SEQ ID NO: 504 - protein, 13 aa, Homo sapiens
IIHRGKPFQLEAV

SEQ ID NO: 505 - protein, 19 aa, Homo sapiens
KGQQYDIKYTWNVPKIAPK

SEQ ID NO: 506 - protein, 15 aa, Homo sapiens
KIEIKASIDGLEVDV

SEQ ID NO: 507 - protein, 27 aa, Homo sapiens
KIEIKASIDGLEVDVPGIDPNACHYMK

SEQ ID NO: 508 - protein, 15 aa, Homo sapiens
KPFQLEAVFEANQNT

SEQ ID NO: 509 - protein, 15 aa, Homo sapiens
LEVDVPGIDPNACHY

SEQ ID NO: 510 - protein, 26 aa, Homo sapiens
LEVDVPGIDPNACHYMKCPLVKGQQY

SEQ ID NO: 511 - protein, 19 aa, Homo sapiens
MGDDGVLACAIATHAKIRD

SEQ ID NO: 512 - protein, 15 aa, Homo sapiens
NACHYMKCPLVKGQQ

SEQ ID NO: 513 - protein, 16 aa, Homo sapiens
NACHYMKCPLVKGQQY

SEQ ID NO: 514 - protein, 16 aa, Homo sapiens
NVVVTVKVMGDDGVLA

SEQ ID NO: 515 - protein, 30 aa, Homo sapiens
RGKPFQLEAVFEANQNTKTAKIEIKASIDG

SEQ ID NO: 516 - protein, 17 aa, Homo sapiens
RGKPPQLEAVFEAVQNT

SEQ ID NO: 517 - protein, 14 aa, Homo sapiens
SENVVVTVKVMGDD

SEQ ID NO: 518 - protein, 15 aa, Homo sapiens
SENVVVTVKVMGDDG

SEQ ID NO: 519 - protein, 14 aa, Homo sapiens
TKTAKIEIKASIDG

SEQ ID NO: 520 - protein, 15 aa, Homo sapiens
TWNVPKIAPKSENVV

SEQ ID NO: 521 - protein, 15 aa, Homo sapiens
VKGQQYDIKYTWNVP

SEQ ID NO: 522 - protein, 16 aa, Homo sapiens
VKGQQYDIKYTWNVPK

SEQ ID NO: 523 - protein, 14 aa, Homo sapiens
VLACAIATHAKIRD

SEQ ID NO: 524 - protein, 16 aa, Homo sapiens
VLVPGCHGSEPCIIHR

SEQ ID NO: 525 - protein, 14 aa, Homo sapiens
VMGDDGVLACAIAT

SEQ ID NO: 526 - protein, 26 aa, Homo sapiens
VVVTVKVMGDDGVLACAIATHAKIRD

SEQ ID NO: 527 - protein, 14 aa, Homo sapiens
WNVPKIAPKSENVV

SEQ ID NO: 528 - protein, 15 aa, Homo sapiens
PCIIHRGKPFQLEAV

SEQ ID NO: 529 - protein, 32 aa, Homo sapiens
APKSENVVVTVKVMGDNGVLACAIATHAKIRD

SEQ ID NO: 530 - protein, 31 aa, Homo sapiens
CHGSEPCIIHRGKPFQLEAVFEANQNSKTAK

SEQ ID NO: 531 - protein, 33 aa, Homo sapiens
DQVDVKDCANHEIKKVLVPGCHGSEPCIIHRGK

SEQ ID NO: 532 - protein, 32 aa, Homo sapiens
EANQNSKTAKIEIKASIEGLEVDVPGIDPNAC

SEQ ID NO: 533 - protein, 42 aa, Homo sapiens
EVDVPGIDPNACHYMKCPLVKGQQYDIKYTWIVPKIAPKSEN

SEQ ID NO: 534 - protein, 11 aa, Homo sapiens
DIKYTWNVPKI

SEQ ID NO: 535 - protein, 12 aa, Homo sapiens
DIKYTWNVPKIA

SEQ ID NO: 536 - protein, 17 aa, Homo sapiens
DIKYTWNVPKIAPKSEN

SEQ ID NO: 537 - protein, 16 aa, Homo sapiens
IKYTWNVPKIAPKSEN

SEQ ID NO: 538 - protein, 14 aa, Homo sapiens
QQYDIKYTWNVPKI

SEQ ID NO: 539 - protein, 15 aa, Homo sapiens
QQYDIKYTWNVPKIA

SEQ ID NO: 540 - protein, 16 aa, Homo sapiens
QQYDIKYTWNVPKIAP

SEQ ID NO: 541 - protein, 17 aa, Homo sapiens
QQYDIKYTWNVPKIAPK

SEQ ID NO: 542 - protein, 18 aa, Homo sapiens
QQYDIKYTWNVPKIAPKS

SEQ ID NO: 543 - protein, 19 aa, Homo sapiens
QQYDIKYTWNVPKIAPKSE

SEQ ID NO: 544 - protein, 20 aa, Homo sapiens
QQYDIKYTWNVPKIAPKSEN

SEQ ID NO: 545 - protein, 12 aa, Homo sapiens
QYDIKYTWNVPK

SEQ ID NO: 546 - protein, 13 aa, Homo sapiens
QYDIKYTWNVPKI

SEQ ID NO: 547 - protein, 19 aa, Homo sapiens
QYDIKYTWNVPKIAPKSEN

SEQ ID NO: 548 - protein, 11 aa, Homo sapiens
YDIKYTWNVPK

SEQ ID NO: 549 - protein, 12 aa, Homo sapiens
YDIKYTWNVPKI

SEQ ID NO: 550 - protein, 13 aa, Homo sapiens
YDIKYTWNVPKIA

SEQ ID NO: 551 - protein, 18 aa, Homo sapiens
YDIKYTWNVPKIAPKSEN

SEQ ID NO: 552 - protein, 15 aa, Homo sapiens
EYWILTAAHCVAGQT

SEQ ID NO: 553 - protein, 15 aa, Homo sapiens
GGEKISVAKIFAHEK

SEQ ID NO: 554 - protein, 15 aa, Homo sapiens
IDNDIALIKLKSPMK

SEQ ID NO: 555 - protein, 15 aa, Homo sapiens
IRYNSLKHSLGGEKI

SEQ ID NO: 556 - protein, 15 aa, Homo sapiens
SELRRVDIAVVSRKE

SEQ ID NO: 557 - protein, 15 aa, Homo sapiens
TLANPILPASPNATI

SEQ ID NO: 558 - protein, 15 aa, Homo sapiens
FDFLLMERIHEQIKK

SEQ ID NO: 559 - protein, 15 aa, Homo sapiens
GELALFYLQEQINHF

SEQ ID NO: 560 - protein, 15 aa, Homo sapiens
RKDLDIFEQYNLEMA

SEQ ID NO: 561 - protein, 15 aa, Homo sapiens
DFLLMQRIHEQMKKG

SEQ ID NO: 562 - protein, 15 aa, Homo sapiens
FERYNVEIALKSNEI

SEQ ID NO: 563 - protein, 15 aa, Homo sapiens
LIDGVRGVLNRLMKR

SEQ ID NO: 564 - protein, 15 aa, Homo sapiens
RGVLNRLMKRTDLDI

SEQ ID NO: 565 - protein, 15 aa, Homo sapiens
VEIALKSNEILERDL

SEQ ID NO: 566 - protein, 15 aa, Homo sapiens
YQNEFDFLLMQRIHE

SEQ ID NO: 567 - protein, 31 aa, Homo sapiens
DGVRGVLDRLMQRKDLDIFEQYNLEMAKKSG

SEQ ID NO: 568 - protein, 29 aa, Homo sapiens
DYQNEFDFLLMERIHEQIKKGELALFYLQ

SEQ ID NO: 569 - protein, 31 aa, Homo sapiens
EQYNLEMAKKSGDILERDLKKEEARVKKIEV

SEQ ID NO: 570 - protein, 36 aa, Homo sapiens
KKGELALFYLQEQINHFEEKPTKEMKDKIVAEMDTI

SEQ ID NO: 571 - protein, 30 aa, Homo sapiens
DPIHYDKITEEINKAVDEAVAAIEKSETFD

SEQ ID NO: 572 - protein, 31 aa, Homo sapiens
LKGELDMRNIQVRGLKQMKRVGDANVKSEDG

SEQ ID NO: 573 - protein, 29 aa, Homo sapiens
TAIFQDTVRAEMTKVLAPAFKKELERNNQ

SEQ ID NO: 574 - protein, 31 aa, Homo sapiens
VAAIEKSETFDPMKVPDHSDKFERHIGIIDL

SEQ ID NO: 575 - protein, 26 aa, Homo sapiens
VELSLEVSEEGNMTLTSFEVRQFANV

SEQ ID NO: 576 - protein, 36 aa, Homo sapiens
VHDDVVSMEYDLAYKLGDLHPNTHVISDIQDFVVEL

SEQ ID NO: 577 - protein, 28 aa, Homo sapiens
VNHIGGLSILDPIFAVLSDVLTAIFQDT

SEQ ID NO: 578 - protein, 29 aa, Homo sapiens
GYFADPKDPHKFYICSNWEAVHKDCPGNT

SEQ ID NO: 579 - protein, 29 aa, Homo sapiens
KFYICSNWEAVHKDCPGNTRWNEDEETCT

SEQ ID NO: 580 - protein, 39 aa, Homo sapiens
GYFADPKDPHKFYICSNWEAVHKDCPGNTRWNEDEETCT

SEQ ID NO: 581 - protein, 32 aa, Homo sapiens
MANDNDDDPTTTVHPTTTEQPDDKFECPSRFG

SEQ ID NO: 582 - protein, 34 aa, Homo sapiens
PTTTEQPDDKFECPSRFGYFADPKDPHKFYICSN

SEQ ID NO: 583 - protein, 20 aa, Homo sapiens
DLRQMRTVTPIRMQGGCGSC

SEQ ID NO: 584 - protein, 20 aa, Homo sapiens
GCGSCWAFSGVAATESAYLA

SEQ ID NO: 585 - protein, 15 aa, Homo sapiens
QESYYRYVAREQSCR

SEQ ID NO: 586 - protein, 15 aa, Homo sapiens
RFGISNYCQIYPPNA

SEQ ID NO: 587 - protein, 15 aa, Homo sapiens
HAVNIVGYSNAQGVD

SEQ ID NO: 588 - protein, 15 aa, Homo sapiens
IEYIQHNGVVQESYY

SEQ ID NO: 589 - protein, 15 aa, Homo sapiens
VKYVQSNGGAINHLS

SEQ ID NO: 590 - protein, 15 aa, Homo sapiens
NKIREALAQTHSAIA

SEQ ID NO: 591 - protein, 19 aa, Homo sapiens
DLNAETNACSINGNAPAEI

SEQ ID NO: 592 - protein, 20 aa, Homo sapiens
KDLDAFRHYDGRTIIQRDNG

SEQ ID NO: 593 - protein, 20 aa, Homo sapiens
QELVDCASQHGCHGDTIPRG

SEQ ID NO: 594 - protein, 15 aa, Homo sapiens
NWGDNGYGYFAANID

SEQ ID NO: 595 - protein, 15 aa, Homo sapiens
VIIGIKDLDAFRHYD

SEQ ID NO: 596 - protein, 15 aa, Homo sapiens
INHLSDLSLDEFKNR

SEQ ID NO: 597 - protein, 15 aa, Homo sapiens
AANIDLMMIEEYPYV

SEQ ID NO: 598 - protein, 20 aa, Homo sapiens
CHGSEPCIIHRGKPFQLEAV

SEQ ID NO: 599 - protein, 20 aa, Homo sapiens
YDIKYTWNVPKIAPKSENVV

SEQ ID NO: 600 - protein, 15 aa, Homo sapiens
NTKTAKIEIKASIDG

SEQ ID NO: 601 - protein, 15 aa, Homo sapiens
GVLACAIATHAKIRD

SEQ ID NO: 602 - protein, 15 aa, Homo sapiens
DQVDVKDCANHEIKK

SEQ ID NO: 603 - protein, 15 aa, Homo sapiens
QLEAVFEANQNTKTA

SEQ ID NO: 604 - protein, 15 aa, Homo sapiens
VKGQQYDIKYTWNVP

SEQ ID NO: 605 - protein, 15 aa, Homo sapiens
NACHYMKCPLVKGQQ

SEQ ID NO: 606 - protein, 20 aa, Homo sapiens
PKDPHKFYICSNWEAVHKDC

SEQ ID NO: 607 - protein, 15 aa, Homo sapiens
CPSRFGYFADPKDPH

SEQ ID NO: 608 - protein, 41 aa, Homo sapiens; identical to SEQ ID NO: 487

SEQ ID NO: 609 - protein, 43 aa, Homo sapiens; identical to SEQ ID NO: 486

SEQ ID NO: 610 - protein, 31 aa, Homo sapiens; identical to SEQ ID NO: 482

SEQ ID NO: 611 - protein, 37 aa, Homo sapiens; identical to SEQ ID NO: 484

SEQ ID NO: 612 - protein, 31 aa, Homo sapiens; identical to SEQ ID NO: 483

SEQ ID NO: 613 - protein, 33 aa, Homo sapiens; identical to SEQ ID NO: 481

SEQ ID NO: 614 - protein, 34 aa, Homo sapiens; identical to SEQ ID NO: 485

SEQ ID NO: 615 - protein, 35 aa, Homo sapiens; identical to SEQ ID NO: 488

SEQ ID NO: 616 - protein, 33 aa, Homo sapiens; identical to SEQ ID NO: 531

SEQ ID NO: 617 - protein, 31 aa, Homo sapiens; identical to SEQ ID NO: 530

SEQ ID NO: 618 - protein, 32 aa, Homo sapiens; identical to SEQ ID NO: 532

SEQ ID NO: 619 - protein, 42 aa, Homo sapiens; identical to SEQ ID NO: 533

SEQ ID NO: 620 - protein, 32 aa, Homo sapiens; identical to SEQ ID NO: 529

SEQ ID NO: 621 - protein, 30 aa, Homo sapiens
CDYQNEFDFLLMERIHEQIKKGELALFYLQ

SEQ ID NO: 622 - protein, 37 aa, Homo sapiens
CKKGELALFYLQEQINHFEEKPTKEMKDKIVAEMDTI

SEQ ID NO: 623 - protein, 32 aa, Homo sapiens
CDGVRGVLDRLMQRKDLDIFEQYNLEMAKKSG

SEQ ID NO: 624 - protein, 32 aa, Homo sapiens
CEQYNLEMAKKSGDILERDLKKEEARVKKIEV

SEQ ID NO: 625 - protein, 31 aa, Homo sapiens
CDPIHYDKITEEINKAVDEAVAAIEKSETFD

SEQ ID NO: 626 - protein, 32 aa, Homo sapiens
CVAAIEKSETFDPMKVPDHSDKFERHIGIIDL

SEQ ID NO: 627 - protein, 32 aa, Homo sapiens
CLKGELDMRNIQVRGLKQMKRVGDANVKSEDG

SEQ ID NO: 628 - protein, 37 aa, Homo sapiens
CVHDDVVSMEYDLAYKLGDLHPNTHVISDIQDFVVEL

SEQ ID NO: 629 - protein, 27 aa, Homo sapiens
CVELSLEVSEEGNMTLTSFEVRQFANV

SEQ ID NO: 630 - protein, 29 aa, Homo sapiens
CVNHIGGLSILDPIFAVLSDVLTAIFQDT

SEQ ID NO: 631 - protein, 30 aa, Homo sapiens
CTAIFQDTVRAEMTKVLAPAFKKELERNNQ

SEQ ID NO: 632 - protein, 35 aa, Homo sapiens
CFIVGDKKEDEWRMAFDRLMMEELETKIDQVEKGL

SEQ ID NO: 633 - protein, 38 aa, Homo sapiens
CLHLSEQYKELEKTKSKELKEQILRELTIGENFMKGAL

SEQ ID NO: 634 - protein, 30 aa, Homo sapiens
CMKGALKFFEMEAKRTDLNMFERYNYEFAL

SEQ ID NO: 635 - protein, 34 aa, Homo sapiens
CYNYEFALESIKLLIKKLDELAKKVKAVNPDEYY

SEQ ID NO: 636 - protein, 32 aa, Homo sapiens; identical to SEQ ID NO: 581

SEQ ID NO: 637 - protein, 34 aa, Homo sapiens; identical to SEQ ID NO: 582

SEQ ID NO: 638 - protein, 39 aa, Homo sapiens; identical to SEQ ID NO: 580

SEQ ID NO: 639 - protein, 29 aa, Homo sapiens
GYFADPKDPHKFYICSNWEAVHKDCPGNT

SEQ ID NO: 640 - protein, 29 aa, Homo sapiens
KFYICSNWEAVHKDCPGNTRWNEDEETCT

SEQ ID NO: 641 - protein, 15 aa, Homo sapiens
QESYYRYVAREQSCR

SEQ ID NO: 642 - protein, 20 aa, Homo sapiens
KDLDAFRHYDGRTIIQRDNG

SEQ ID NO: 643 - protein, 15 aa, Homo sapiens
DQVDVKDCANHEIKK

SEQ ID NO: 644 - protein, 15 aa, Homo sapiens
VKGQQYDIKYTWNVP

SEQ ID NO: 645 - protein, 20 aa, Homo sapiens
PKDPHKFYICSNWEAVHKDC

SEQ ID NO: 646 - protein, 15 aa, Homo sapiens
CPSRFGYFADPKDPH

SEQ ID NO: 647 - protein, 15 aa, Homo sapiens
QESYYRYVAREQSCR

SEQ ID NO: 648 - DNA, 45 bp, Homo sapiens
  source 1..45: mol_type = other DNA
caggaaagctattatcgctatgtggcgcgcgaacagagctgccgc

SEQ ID NO: 649 - protein, 20 aa, Homo sapiens
KDLDAFRHYDGRTIIQRDNG

SEQ ID NO: 650 - DNA, 60 bp, Homo sapiens
  source 1..60: mol_type = other DNA
aaagatctggatgcgtttcgccattatgatggccgcaccattattcagcgcgataacggc

SEQ ID NO: 651 - protein, 15 aa, Homo sapiens
DQVDVKDCANHEIKK

SEQ ID NO: 652 - DNA, 45 bp, Homo sapiens
  source 1..45: mol_type = other DNA
gatcaggtggatgtgaaagattgcgcgaaccatgaaattaaaaaa

SEQ ID NO: 653 - protein, 15 aa, Homo sapiens
VKGQQYDIKYTWNVP

SEQ ID NO: 654 - DNA, 45 bp, Homo sapiens
  source 1..45: mol_type = other DNA
gtgaaaggccagcagtatgatattaaatatacctggaacgtgccg

SEQ ID NO: 655 - protein, 20 aa, Homo sapiens
PKDPHKFYICSNWEAVHKDC

SEQ ID NO: 656 - DNA, 60 bp, Homo sapiens
  source 1..60: mol_type = other DNA
ccgaaagatccgcataaattttatatttgcagcaactgggaagcggtgcataaagattgc

SEQ ID NO: 657 - protein, 15 aa, Homo sapiens
CPSRFGYFADPKDPH

SEQ ID NO: 658 - DNA, 45 bp, Homo sapiens
  source 1..45: mol_type = other DNA
tgcccgagccgctttggctattttgcggatccgaaagatccgcat

SEQ ID NO: 659 - DNA, 921 bp, synthetic construct
  misc_feature 1..921: note = Codon-optimized DNA encoding human Der p 1, p 2, and p 23 T cell epitopes
  source 1..921: mol_type = other DNA
atgatggaccaggccagaagcgccttctccaacctcttcggaggcgagcctctgagctacaccagattcagcctggctagacaggtggatggcgacaacagccacgtggaaatgaagctggctgtggacgaggaagagaacgccgataacaacaccaaggccaatgtgacaaagcctaaaagatgcagcggctccatctgctacggcacaatcgccgtgatcgtgttcttcctgatcggatttatgatcggctacctgggctactgtaaaggcgtggaacctaagaccgagtgcgagagactggctggcacagagagcccagtgcgggaagaaccaggagaagatttccccgccggcggcggcggatctagagtgcggagaggaggaggcggcagccaggagtcttattacagatacgtggccagagagcagagctgcagaggcggcggaggctctagagtgcggcggggcggcgggggcagcaaggatctggacgcctttagacactacgacggcagaaccattatccagcgggacaatggcggcggcggtggcagcagagtgcggcggggaggcggcggctctgatcaggttgacgtgaaggactgtgccaaccacgagatcaaaaagggcggcggtggcagcagagtgagaagaggcggcgggggcagcgtgaagggccagcaatacgacatcaagtacacctggaacgtgcccggaggcggaggcagccgcgtcagaaggggcggtggaggctctcctaaagacccccacaagttctacatctgcagcaactgggaggccgtgcacaaggactgcggcggcggcggcagcagagtccgacgtggcggaggcggctcctgtcctagccggttcggctatttcgccgaccctaaggatcctcatcgggtgcggagatga

SEQ ID NO: 660 - protein, 303 aa, Arachis hypogaea
ARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

SEQ ID NO: 661 - protein, 15 aa, Mus musculus
GCGSCWAFSGVAATE

SEQ ID NO: 662 - protein, 15 aa, Mus musculus
DNGYGYFAANIDLMM

SEQ ID NO: 663 - protein, 15 aa, Mus musculus
IREALAQTHSAIAVI

SEQ ID NO: 664 - protein, 15 aa, Mus musculus
SNYCQIYPPNVNKIR

SEQ ID NO: 665 - protein, 15 aa, Mus musculus
SVKYVQSNGGAINHL

SEQ ID NO: 666 - protein, 15 aa, Mus musculus
EEYKKAFNKSYATFE

SEQ ID NO: 667 - protein, 129 aa, Arachis hypogaea
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 668 - protein, 15 aa, Mus musculus
DIKYTWNVPKIAPKS

SEQ ID NO: 669 - protein, 15 aa, Mus musculus
DDGVLACAIATHAKI

SEQ ID NO: 670 - protein, 15 aa, Mus musculus
LEAVFEANQNTKTAK

SEQ ID NO: 671 - protein, 69 aa, Arachis hypogaea
ANDNDDDPTTTVHPTTTEQPDDKFECPSRFGYFADPKDPHKFYICSNWEAVHKDCPGNTRWNEDEETCT

SEQ ID NO: 672 - protein, 15 aa, Arachis hypogaea
DDPTTTVHPTTTEQP

SEQ ID NO: 673 - protein, 15 aa, Arachis hypogaea
SRFGYFADPKDPHKF

SEQ ID NO: 674 - DNA, 45 bp, Arachis hypogaea
  source 1..45: mol_type = other DNA
agcaactattgccagatttatccgccgaacgtgaacaaaattcgc

SEQ ID NO: 675 - DNA, 45 bp, Arachis hypogaea
  source 1..45: mol_type = other DNA
gaagaatataaaaaagcgtttaacaaaagctatgcgacctttgaa

SEQ ID NO: 676 - DNA, 45 bp, Arachis hypogaea
  source 1..45: mol_type = other DNA
gatattaaatatacctggaacgtgccgaaaattgcgccgaaaagc

SEQ ID NO: 677 - DNA, 45 bp, Arachis hypogaea
  source 1..45: mol_type = other DNA
ctggaagcggtgtttgaagcgaaccagaacaccaaaaccgcgaaa

SEQ ID NO: 678 - DNA, 45 bp, Arachis hypogaea
  source 1..45: mol_type = other DNA
gatgatccgaccaccaccgtgcatccgaccaccaccgaacagccg

SEQ ID NO: 679 - DNA, 45 bp, Arachis hypogaea
  source 1..45: mol_type = other DNA
agccgctttggctattttgcggatccgaaagatccgcataaattt

SEQ ID NO: 680 - DNA, 894 bp, synthetic construct
  misc_feature 1..894: note = Codon optimized DNA encoding mouse TFR 1-118 and DER P 1, 2, 23 T cell epitopes
  source 1..894: mol_type = other DNA
atgatgatggaccaggctcgcagtgctttcagtaacctgtttggaggggagcccctatcctacaccaggttcagcttggccaggcaagtggatggagacaattcacatgtggagatgaaacttgccgcggatgaggaggaaaatgccgacaacaatatgaaggcatcagtgcggaaaccaaagcgcttcaatggcaggctgtgctttgctgctatagccctggttattttcttcctcattggcttcatgtccggctatttagggtattgtaagagagtagaacaaaaagaagagtgtgtgaagctggcagagactgaggagacagataaaagcgagaccatggagacagaagatgtgggcggaggcgggtcccgggtgagacgtgggggaggtggaagttcaaactactgccagatctaccctccgaatgtgaacaagatccgcgggggtggcgggagccgggtccgacgcgggggaggtgggtctgaagaatacaagaaggccttcaacaaatcttatgcgacctttgagggcggtggcgggagcagagtcagacggggagggggcggaagcgacatcaagtacacctggaatgttcctaaaattgcccctaagtctggcgggggcggatcgagggtcagaagaggcggaggcgggtctctcgaagcagtatttgaagccaaccagaacaccaagacagctaaaggtgggggaggttccagggtcaggcgcggaggtggtggctctgatgatcctacaactactgttcacccaactacgacggagcagcccggaggcggtggctcccgtgtgagacgaggtggaggcgggtcaagtcgatttggctattttgcagacccaaaagacccccacaagttccgagtccggcggtga

SEQ ID NO: 681 - protein, 20 aa, Homo sapiens
TAVYYCAKVSYLSTASSLDY

SEQ ID NO: 682 - protein, 15 aa, Homo sapiens
CAKVSYLSTASSLDY

SEQ ID NO: 683 - protein, 129 aa, synthetic construct
  REGION 1..129: note = Adalimumab antibody heavy chain protein sequence
EVQLVESGGGLVQPGSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVF

SEQ ID NO: 684 - protein, 15 aa, Homo sapiens
AKNSLYLQMNSLRAE

SEQ ID NO: 685 - protein, 15 aa, Homo sapiens
KNSLYLQMNSLRAED

SEQ ID NO: 686 - protein, 15 aa, Homo sapiens
NAKNSLYLQMNSLRA

SEQ ID NO: 687 - protein, 15 aa, Homo sapiens
NSLYLQMNSLRAEDT

SEQ ID NO: 688 - protein, 15 aa, Homo sapiens
SLYLQMNSLRAEDTA

SEQ ID NO: 689 - protein, 15 aa, Homo sapiens
AKNSLYLQMNSLRAE

SEQ ID NO: 690 - protein, 15 aa, Homo sapiens
KNSLYLQMNSLRAED

SEQ ID NO: 691 - protein, 15 aa, Homo sapiens
NAKNSLYLQMNSLRA

SEQ ID NO: 692 - protein, 15 aa, Homo sapiens
NSLYLQMNSLRAEDT

SEQ ID NO: 693 - protein, 15 aa, Homo sapiens
SLYLQMNSLRAEDTA

SEQ ID NO: 694 - protein, 15 aa, Homo sapiens
GQGTLVTVSSASTKG

SEQ ID NO: 695 - protein, 15 aa, Homo sapiens
QGTLVTVSSASTKGP

SEQ ID NO: 696 - protein, 15 aa, Homo sapiens
GTLVTVSSASTKGPS

SEQ ID NO: 697 - protein, 15 aa, Homo sapiens
LYLQMNSLRAEDTAV

SEQ ID NO: 698 - protein, 15 aa, Homo sapiens
YLQMNSLRAEDTAVY

SEQ ID NO: 699 - protein, 15 aa, Homo sapiens
CAKVSYLSTASSLDY

SEQ ID NO: 700 - protein, 15 aa, Homo sapiens
YCAKVSYLSTASSLD

SEQ ID NO: 701 - protein, 15 aa, Homo sapiens
AKVSYLSTASSLDYW

SEQ ID NO: 702 - protein, 15 aa, Homo sapiens
WGQGTLVTVSSASTK

SEQ ID NO: 703 - protein, 15 aa, Homo sapiens
KVSYLSTASSLDYWG

SEQ ID NO: 704 - protein, 15 aa, Homo sapiens
VSYLSTASSLDYWGQ

SEQ ID NO: 705 - protein, 15 aa, Homo sapiens
LYLQMNSLRAEDTAV

SEQ ID NO: 706 - protein, 15 aa, Homo sapiens
YLQMNSLRAEDTAVY

SEQ ID NO: 707 - protein, 15 aa, Homo sapiens
AKVSYLSTASSLDYW

SEQ ID NO: 708 - protein, 15 aa, Homo sapiens
AKNSLYLQMNSLRAE

SEQ ID NO: 709 - protein, 15 aa, Homo sapiens
CAKVSYLSTASSLDY

SEQ ID NO: 710 - protein, 15 aa, Homo sapiens
KNSLYLQMNSLRAED

SEQ ID NO: 711 - protein, 15 aa, Homo sapiens
NSLYLQMNSLRAEDT

SEQ ID NO: 712 - protein, 15 aa, Homo sapiens
CAASGFTFDDYAMHW

SEQ ID NO: 713 - protein, 15 aa, Homo sapiens
YCAKVSYLSTASSLD

SEQ ID NO: 714 - protein, 15 aa, Homo sapiens
YYCAKVSYLSTASSL

SEQ ID NO: 715 - protein, 15 aa, Homo sapiens
AKNSLYLQMNSLRAE

SEQ ID NO: 716 - protein, 15 aa, Homo sapiens
AKNSLYLQMNSLRAE

SEQ ID NO: 717 - protein, 15 aa, Homo sapiens
KNSLYLQMNSLRAED

SEQ ID NO: 718 - protein, 15 aa, Homo sapiens
KNSLYLQMNSLRAED

SEQ ID NO: 719 - protein, 15 aa, Homo sapiens
LYLQMNSLRAEDTAV

SEQ ID NO: 720 - protein, 15 aa, Homo sapiens
NAKNSLYLQMNSLRA

SEQ ID NO: 721 - protein, 15 aa, Homo sapiens
NAKNSLYLQMNSLRA

SEQ ID NO: 722 - protein, 15 aa, Homo sapiens
NSLYLQMNSLRAEDT

SEQ ID NO: 723 - protein, 15 aa, Homo sapiens
NSLYLQMNSLRAEDT

SEQ ID NO: 724 - protein, 15 aa, Homo sapiens
SLYLQMNSLRAEDTA

SEQ ID NO: 725 - protein, 15 aa, Homo sapiens
YLQMNSLRAEDTAVY

SEQ ID NO: 726 - protein, 15 aa, Homo sapiens
SLYLQMNSLRAEDTA

SEQ ID NO: 727 - protein, 15 aa, Homo sapiens
TLVTVSSASTKGPSV

SEQ ID NO: 728 - protein, 15 aa, Homo sapiens
KVSYLSTASSLDYWG

SEQ ID NO: 729 - protein, 15 aa, Homo sapiens
MNSLRAEDTAVYYCA

SEQ ID NO: 730 - protein, 15 aa, Homo sapiens
PGSLRLSCAASGFTF

SEQ ID NO: 731 - protein, 15 aa, Homo sapiens
SCAASGFTFDDYAMH

SEQ ID NO: 732 - protein, 15 aa, Homo sapiens
VYYCAKVSYLSTASS

SEQ ID NO: 733 - protein, 15 aa, Homo sapiens
NSLRAEDTAVYYCAK

SEQ ID NO: 734 - protein, 15 aa, Homo sapiens
QGTLVTVSSASTKGP

SEQ ID NO: 735 - protein, 15 aa, Homo sapiens
GQGTLVTVSSASTKG

SEQ ID NO: 736 - protein, 15 aa, Homo sapiens
YCAKVSYLSTASSLD

SEQ ID NO: 737 - protein, 15 aa, Homo sapiens
YYCAKVSYLSTASSL

SEQ ID NO: 738 - protein, 15 aa, Homo sapiens
GSLRLSCAASGFTFD

SEQ ID NO: 739 - protein, 15 aa, Homo sapiens
QMNSLRAEDTAVYYC

SEQ ID NO: 740 - protein, 15 aa, Homo sapiens
SLYLQMNSLRAEDTA

SEQ ID NO: 741 - protein, 15 aa, Homo sapiens
GTLVTVSSASTKGPS

SEQ ID NO: 742 - protein, 15 aa, Homo sapiens
AKVSYLSTASSLDYW

SEQ ID NO: 743 - protein, 15 aa, Homo sapiens
LYLQMNSLRAEDTAV

SEQ ID NO: 744 - protein, 15 aa, Homo sapiens
VQLVESGGGLVQPGS

SEQ ID NO: 745 - protein, 15 aa, Homo sapiens
YWGQGTLVTVSSAST

SEQ ID NO: 746 - protein, 15 aa, Homo sapiens
CAKVSYLSTASSLDY

SEQ ID NO: 747 - protein, 15 aa, Homo sapiens
NAKNSLYLQMNSLRA

SEQ ID NO: 748 - protein, 15 aa, Homo sapiens
NAKNSLYLQMNSLRA

SEQ ID NO: 749 - protein, 15 aa, Homo sapiens
QLVESGGGLVQPGSL

SEQ ID NO: 750 - protein, 15 aa, Homo sapiens
QPGSLRLSCAASGFT

SEQ ID NO: 751 - protein, 15 aa, Homo sapiens
AKNSLYLQMNSLRAE

SEQ ID NO: 752 - protein, 15 aa, Homo sapiens
KNSLYLQMNSLRAED

SEQ ID NO: 753 - protein, 15 aa, Homo sapiens
NSLYLQMNSLRAEDT

SEQ ID NO: 754 - protein, 15 aa, Homo sapiens
AASGFTFDDYAMHWV

SEQ ID NO: 755 - protein, 15 aa, Homo sapiens
LQMNSLRAEDTAVYY

SEQ ID NO: 756 - protein, 15 aa, Homo sapiens
LVESGGGLVQPGSLR

SEQ ID NO: 757 - protein, 15 aa, Homo sapiens
WGQGTLVTVSSASTK

SEQ ID NO: 758 - protein, 15 aa, Homo sapiens
NAKNSLYLQMNSLRA

SEQ ID NO: 759 - protein, 15 aa, Homo sapiens
VYYCAKVSYLSTASS

SEQ ID NO: 760 - protein, 15 aa, Homo sapiens
VSYLSTASSLDYWGQ

SEQ ID NO: 761 - protein, 15 aa, Homo sapiens
AKVSYLSTASSLDYW

SEQ ID NO: 762 - protein, 15 aa, Homo sapiens
ASGFTFDDYAMHWVR

SEQ ID NO: 763 - protein, 15 aa, Homo sapiens
VESGGGLVQPGSLRL

SEQ ID NO: 764 - protein, 15 aa, Homo sapiens
AASGFTFDDYAMHWV

SEQ ID NO: 765 - protein, 15 aa, Homo sapiens
AKVSYLSTASSLDYW

SEQ ID NO: 766 - protein, 15 aa, Homo sapiens
ASGFTFDDYAMHWVR

SEQ ID NO: 767 - protein, 15 aa, Homo sapiens
EVQLVESGGGLVQPG

SEQ ID NO: 768 - protein, 15 aa, Homo sapiens
CAASGFTFDDYAMHW

SEQ ID NO: 769 - protein, 15 aa, Homo sapiens
LQMNSLRAEDTAVYY

SEQ ID NO: 770 - protein, 15 aa, Homo sapiens
LRLSCAASGFTFDDY

SEQ ID NO: 771 - protein, 15 aa, Homo sapiens
LSCAASGFTFDDYAM

SEQ ID NO: 772 - protein, 15 aa, Homo sapiens
MNSLRAEDTAVYYCA

SEQ ID NO: 773 - protein, 15 aa, Homo sapiens
NSLRAEDTAVYYCAK

SEQ ID NO: 774 - protein, 15 aa, Homo sapiens
QMNSLRAEDTAVYYC

SEQ ID NO: 775 - protein, 15 aa, Homo sapiens
RLSCAASGFTFDDYA

SEQ ID NO: 776 - protein, 15 aa, Homo sapiens
AKNSLYLQMNSLRAE

SEQ ID NO: 777 - protein, 15 aa, Homo sapiens
GKGLEWVSAITWNSG

SEQ ID NO: 778 - protein, 15 aa, Homo sapiens
KGLEWVSAITWNSGH

SEQ ID NO: 779 - protein, 15 aa, Homo sapiens
KNSLYLQMNSLRAED

SEQ ID NO: 780 - protein, 15 aa, Homo sapiens
SGFTFDDYAMHWVRQ

SEQ ID NO: 781 - protein, 15 aa, Homo sapiens
WVSAITWNSGHIDYA

SEQ ID NO: 782 - protein, 15 aa, Homo sapiens
CAKVSYLSTASSLDY

SEQ ID NO: 783 - protein, 15 aa, Homo sapiens
AKVSYLSTASSLDYW

SEQ ID NO: 784 - protein, 15 aa, Homo sapiens
CAKVSYLSTASSLDY

SEQ ID NO: 785 - protein, 15 aa, Homo sapiens
GRFTISRDNAKNSLY

SEQ ID NO: 786 - protein, 15 aa, Homo sapiens
VQPGSLRLSCAASGF

SEQ ID NO: 787 - protein, 15 aa, Homo sapiens
GSLRLSCAASGFTFD

SEQ ID NO: 788 - protein, 120 aa, synthetic construct
  REGION 1..120: note = Adalimumab antibody light chain protein sequence
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPP

SEQ ID NO: 789 - protein, 15 aa, Homo sapiens
KVEIKRTVAAPSVFI

SEQ ID NO: 790 - protein, 15 aa, Homo sapiens
VEIKRTVAAPSVFIF

SEQ ID NO: 791 - protein, 15 aa, Homo sapiens
EIKRTVAAPSVFIFP

SEQ ID NO: 792 - protein, 15 aa, Homo sapiens
TKVEIKRTVAAPSVF

SEQ ID NO: 793 - protein, 15 aa, Homo sapiens
IKRTVAAPSVFIFPP

SEQ ID NO: 794 - protein, 15 aa, Homo sapiens
GTKVEIKRTVAAPSV

SEQ ID NO: 795 - protein, 15 aa, Homo sapiens
KLLIYAASTLQSGVP

SEQ ID NO: 796 - protein, 15 aa, Homo sapiens
LLIYAASTLQSGVPS

SEQ ID NO: 797 - protein, 15 aa, Homo sapiens
PKLLIYAASTLQSGV

SEQ ID NO: 798 - protein, 15 aa, Homo sapiens
TKVEIKRTVAAPSVF

SEQ ID NO: 799 - protein, 15 aa, Homo sapiens
APKLLIYAASTLQSG

SEQ ID NO: 800 - protein, 15 aa, Homo sapiens
GKAPKLLIYAASTLQ

SEQ ID NO: 801 - protein, 15 aa, Homo sapiens
GTKVEIKRTVAAPSV

SEQ ID NO: 802 - protein, 15 aa, Homo sapiens
KAPKLLIYAASTLQS

SEQ ID NO: 803 - protein, 15 aa, Homo sapiens
PGKAPKLLIYAASTL

SEQ ID NO: 804 - protein, 15 aa, Homo sapiens
PKLLIYAASTLQSGV

SEQ ID NO: 805 - protein, 15 aa, Homo sapiens
CRASQGIRNYLAWYQ

SEQ ID NO: 806 - protein, 15 aa, Homo sapiens
KVEIKRTVAAPSVFI

SEQ ID NO: 807 - protein, 15 aa, Homo sapiens
LIYAASTLQSGVPSR

SEQ ID NO: 808 - protein, 15 aa, Homo sapiens
PKLLIYAASTLQSGV

SEQ ID NO: 809 - protein, 15 aa, Homo sapiens
RASQGIRNYLAWYQQ

SEQ ID NO: 810 - protein, 15 aa, Homo sapiens
APKLLIYAASTLQSG

SEQ ID NO: 811 - protein, 15 aa, Homo sapiens
APYTFGQGTKVEIKR

SEQ ID NO: 812 - protein, 15 aa, Homo sapiens
NRAPYTFGQGTKVEI

SEQ ID NO: 813 - protein, 15 aa, Homo sapiens
PKLLIYAASTLQSGV

SEQ ID NO: 814 - protein, 15 aa, Homo sapiens
RAPYTFGQGTKVEIK

SEQ ID NO: 815 - protein, 15 aa, Homo sapiens
ASQGIRNYLAWYQQK

SEQ ID NO: 816 - protein, 15 aa, Homo sapiens
KAPKLLIYAASTLQS

SEQ ID NO: 817 - protein, 15 aa, Homo sapiens
KLLIYAASTLQSGVP

SEQ ID NO: 818 - protein, 15 aa, Homo sapiens
KVEIKRTVAAPSVFI

SEQ ID NO: 819 - protein, 15 aa, Homo sapiens
RYNRAPYTFGQGTKV

SEQ ID NO: 820 - protein, 15 aa, Homo sapiens
SQGIRNYLAWYQQKP

SEQ ID NO: 821 - protein, 15 aa, Homo sapiens
YNRAPYTFGQGTKVE

SEQ ID NO: 822 - protein, 15 aa, Homo sapiens
APKLLIYAASTLQSG

SEQ ID NO: 823 - protein, 15 aa, Homo sapiens
GTDFTLTISSLQPED

SEQ ID NO: 824 - protein, 15 aa, Homo sapiens
KLLIYAASTLQSGVP

SEQ ID NO: 825 - protein, 15 aa, Homo sapiens
QGTKVEIKRTVAAPS

SEQ ID NO: 826 - protein, 15 aa, Homo sapiens
KAPKLLIYAASTLQS

SEQ ID NO: 827 - protein, 15 aa, Homo sapiens
QGIRNYLAWYQQKPG

SEQ ID NO: 828 - protein, 15 aa, Homo sapiens
APKLLIYAASTLQSG

SEQ ID NO: 829 - protein, 15 aa, Homo sapiens
DFTLTISSLQPEDVA

SEQ ID NO: 830 - protein, 15 aa, Homo sapiens
PKLLIYAASTLQSGV

SEQ ID NO: 831 - protein, 15 aa, Homo sapiens
TDFTLTISSLQPEDV

SEQ ID NO: 832 - protein, 15 aa, Homo sapiens
FTLTISSLQPEDVAT

SEQ ID NO: 833 - protein, 15 aa, Homo sapiens
TKVEIKRTVAAPSVF

SEQ ID NO: 834 - protein, 15 aa, Homo sapiens
SGTDFTLTISSLQPE

SEQ ID NO: 835 - protein, 15 aa, Homo sapiens
VEIKRTVAAPSVFIF

SEQ ID NO: 836 - protein, 15 aa, Homo sapiens
VEIKRTVAAPSVFIF

SEQ ID NO: 837 - protein, 15 aa, Homo sapiens
LLIYAASTLQSGVPS

SEQ ID NO: 838 - protein, 15 aa, Homo sapiens
APKLLIYAASTLQSG

SEQ ID NO: 839 - protein, 15 aa, Homo sapiens
GTDFTLTISSLQPED

SEQ ID NO: 840 - protein, 15 aa, Homo sapiens
GTDFTLTISSLQPED

SEQ ID NO: 841 - protein, 15 aa, Homo sapiens
IYAASTLQSGVPSRF

SEQ ID NO: 842 - protein, 15 aa, Homo sapiens
KAPKLLIYAASTLQS

SEQ ID NO: 843 - protein, 15 aa, Homo sapiens
KLLIYAASTLQSGVP

SEQ ID NO: 844 - protein, 15 aa, Homo sapiens
KVEIKRTVAAPSVFI

SEQ ID NO: 845 - protein, 15 aa, Homo sapiens
TDFTLTISSLQPEDV

SEQ ID NO: 846 - protein, 15 aa, Homo sapiens
TKVEIKRTVAAPSVF

SEQ ID NO: 847 - protein, 15 aa, Homo sapiens
KAPKLLIYAASTLQS

SEQ ID NO: 848 - protein, 15 aa, Homo sapiens
QGTKVEIKRTVAAPS

SEQ ID NO: 849 - protein, 15 aa, Homo sapiens
APKLLIYAASTLQSG

SEQ ID NO: 850 - protein, 15 aa, Homo sapiens
GKAPKLLIYAASTLQ

SEQ ID NO: 851 - protein, 15 aa, Homo sapiens
KLLIYAASTLQSGVP

SEQ ID NO: 852 - protein, 15 aa, Homo sapiens
QGIRNYLAWYQQKPG

SEQ ID NO: 853 - protein, 15 aa, Homo sapiens
SGTDFTLTISSLQPE

SEQ ID NO: 854 - protein, 15 aa, Homo sapiens
VEIKRTVAAPSVFIF

SEQ ID NO: 855 - protein, 15 aa, Homo sapiens
PKLLIYAASTLQSGV

SEQ ID NO: 856 - protein, 15 aa, Homo sapiens
SQGIRNYLAWYQQKP

SEQ ID NO: 857 - protein, 15 aa, Homo sapiens
GTKVEIKRTVAAPSV

SEQ ID NO: 858 - protein, 15 aa, Homo sapiens
SGTDFTLTISSLQPE

SEQ ID NO: 859 - protein, 15 aa, Homo sapiens
EIKRTVAAPSVFIFP

SEQ ID NO: 860 - protein, 15 aa, Homo sapiens
GTKVEIKRTVAAPSV

SEQ ID NO: 861 - protein, 15 aa, Homo sapiens
GIRNYLAWYQQKPGK

SEQ ID NO: 862 - protein, 15 aa, Homo sapiens
GIRNYLAWYQQKPGK

SEQ ID NO: 863 - protein, 15 aa, Homo sapiens
KVEIKRTVAAPSVFI

SEQ ID NO: 864 - protein, 15 aa, Homo sapiens
VEIKRTVAAPSVFIF

SEQ ID NO: 865 - protein, 15 aa, Homo sapiens
KAPKLLIYAASTLQS

SEQ ID NO: 866 - protein, 15 aa, Homo sapiens
LIYAASTLQSGVPSR

SEQ ID NO: 867 - protein, 15 aa, Homo sapiens
LLIYAASTLQSGVPS

SEQ ID NO: 868 - protein, 15 aa, Homo sapiens
QGIRNYLAWYQQKPG

SEQ ID NO: 869 - protein, 15 aa, Homo sapiens
GKAPKLLIYAASTLQ

SEQ ID NO: 870 - protein, 15 aa, Homo sapiens
PKLLIYAASTLQSGV

SEQ ID NO: 871 - protein, 15 aa, Homo sapiens
EIKRTVAAPSVFIFP

SEQ ID NO: 872 - protein, 15 aa, Homo sapiens
IKRTVAAPSVFIFPP

SEQ ID NO: 873 - protein, 15 aa, Homo sapiens
KVEIKRTVAAPSVFI

SEQ ID NO: 874 - protein, 15 aa, Homo sapiens
TDFTLTISSLQPEDV

SEQ ID NO: 875 - protein, 15 aa, Homo sapiens
TKVEIKRTVAAPSVF

SEQ ID NO: 876 - protein, 15 aa, Homo sapiens
VEIKRTVAAPSVFIF

SEQ ID NO: 877 - protein, 15 aa, Homo sapiens
PGKAPKLLIYAASTL

SEQ ID NO: 878 - protein, 15 aa, Homo sapiens
APKLLIYAASTLQSG

SEQ ID NO: 879 - protein, 15 aa, Homo sapiens
KLLIYAASTLQSGVP

SEQ ID NO: 880 - protein, 15 aa, Homo sapiens
IRNYLAWYQQKPGKA

SEQ ID NO: 881 - protein, 15 aa, Homo sapiens
LLIYAASTLQSGVPS

SEQ ID NO: 882 - protein, 15 aa, Homo sapiens
GTKVEIKRTVAAPSV

SEQ ID NO: 883 - protein, 15 aa, Homo sapiens
KLLIYAASTLQSGVP

SEQ ID NO: 884 - protein, 15 aa, Homo sapiens
PKLLIYAASTLQSGV

SEQ ID NO: 885 - protein, 15 aa, Homo sapiens
DFTLTISSLQPEDVA

SEQ ID NO: 886 - protein, 129 aa, synthetic construct; identical to SEQ ID NO: 683
  REGION 1..129: note = Adalimumab antibody heavy chain protein sequence

SEQ ID NO: 887 - protein, 121 aa, synthetic construct
  REGION 1..121: note = Adalimumab antibody light chain protein sequence
DIQMTQSPSSLSASVGDRVTITCRASQGIRNYLAWYQQKPGKAPKLLIYAASTLQSGVPSRFSGSGSGTDFTLTISSSLQPEDVATYYCQRYNRAPYRFGQGTKVEIKRTVAAPSVFIFPP

SEQ ID NO: 888 - protein, 20 aa, synthetic construct
  REGION 1..20: note = Adalimumab heavy chain T cell epitopes that overlap with CDRs
EWVSAITWNSGHIDYADSVE

SEQ ID NO: 889 - protein, 40 aa, synthetic construct
  REGION 1..40: note = Adalimumab heavy chain T cell epitopes that overlap with CDRs
LQMNSLRAEDTAVYYCAKVSYLSTASSLDYWGQGTLVTVS

SEQ ID NO: 890 - protein, 34 aa, synthetic construct
  REGION 1..34: note = Adalimumab light chain T cell epitopes that overlap with CDRs
TCRASQGIRNYLAWYQQKPGKAPKLLIYAASTLQ

SEQ ID NO: 891 - protein, 35 aa, synthetic construct
  REGION 1..35: note = Adalimumab light chain T cell epitopes that overlap with CDRs
EDVATYYCQRYNRAPYTFGQGTKVEIKRTVAAPSV

SEQ ID NO: 892 - protein, 20 aa, Homo sapiens
EWVSAITWNSGHIDYADSVE

SEQ ID NO: 893 - protein, 20 aa, Homo sapiens
LQMNSLRAEDTAVYYCAKVS

SEQ ID NO: 894 - protein, 20 aa, Homo sapiens
TAVYYCAKVSYLSTASSLDY

SEQ ID NO: 895 - protein, 20 aa, Homo sapiens
YLSTASSLDYWGQGTLVTVS

SEQ ID NO: 896 - protein, 20 aa, Homo sapiens
ITCRASQGIRNYLAWYQQKP

SEQ ID NO: 897 - protein, 20 aa, Homo sapiens
YQQKPGKAPKLLIYAASTLQ

SEQ ID NO: 898 - protein, 20 aa, Homo sapiens
EDVATYYCQRYNRAPYTFGQ

SEQ ID NO: 899 - protein, 20 aa, Homo sapiens
YTFGQGTKVEIKRTVAAPSV

SEQ ID NO: 900 - DNA, 60 bp, Homo sapiens
  source 1..60: mol_type = other DNA
gaatgggtgagcgcgattacctggaacagcggccatattgattatgcggatagcgtggaa

SEQ ID NO: 901 - DNA, 60 bp, Homo sapiens
  source 1..60: mol_type = other DNA
ctgcagatgaacagcctgcgcgcggaagataccgcggtgtattattgcgcgaaagtgagc

SEQ ID NO: 902 - DNA, 60 bp, Homo sapiens
  source 1..60: mol_type = other DNA
accgcggtgtattattgcgcgaaagtgagctatctgagcaccgcgagcagcctggattat

SEQ ID NO: 903 - DNA, 60 bp, Homo sapiens
  source 1..60: mol_type = other DNA
tatctgagcaccgcgagcagcctggattattggggccagggcaccctggtgaccgtgagc

SEQ ID NO: 904 - DNA, 60 bp, Homo sapiens
  source 1..60: mol_type = other DNA
attacctgccgcgcgagccagggcattcgcaactatctggcgtggtatcagcagaaaccg

SEQ ID NO: 905 - DNA, 60 bp, Homo sapiens
  source 1..60: mol_type = other DNA
tatcagcagaaaccgggcaaagcgccgaaactgctgatttatgcggcgagcaccctgcag

SEQ ID NO: 906 - DNA, 60 bp, Homo sapiens
  source 1..60: mol_type = other DNA
gaagatgtggcgacctattattgccagcgctataaccgcgcgccgtatacctttggccag

SEQ ID NO: 907 - DNA, 60 bp, Homo sapiens
  source 1..60: mol_type = other DNA
tatacctttggccagggcaccaaagtggaaattaaacgcaccgtggcggcgccgagcgtg

SEQ ID NO: 908 - DNA, 1188 bp, synthetic construct
  misc_feature 1..1188: note = Codon optimized DNA encoding multi-epitope vaccine for immune tolerization against adalimumab ADA
  source 1..1188: mol_type = other DNA
atgatggaccaggccagaagcgccttcagcaacctgttcggcggcgagcccctgagctacaccagattcagcctggcaagacaggtggacggcgacaacagccacgtggaaatgaaactggccgtggacgaggaagaaaacgccgataacaataccaaggctaatgtgacaaagcccaagagatgcagcggcagcatctgctacggcaccatcgccgtgatcgtgttcttcctgatcggcttcatgatcggatatctgggctactgcaagggcgtggaacctaagaccgagtgcgagagactggccggcacagagagccctgtgagggaagagccaggagaagatttccccgctggcggcggaggtagcagagtgagacggggcggcggaggaagtcaggagtgggtgtccgccatcacatggaacagcggccacatcgactacgccgacagcgtggaaggcggcggaggcagccgggtccggagaggcggcggaggctctctccagatgaactctctgagagccgaggacaccgccgtgtactactgcgccaaggtgagcggcggcggcggcagcagagttagacggggcggcggcggctctacagccgtgtactactgtgccaaggtgtcctatctgtctaccgcctctagcctggactacggaggcggcggtagcagagtcagaagaggaggcggcggaagctacctgagcaccgccagcagcctggattactggggccagggaacactggtgaccgtgtctggaggcggaggatccagagtgcggcggggaggcggcggttccatcacctgcagagccagccagggcattagaaactacctggcctggtaccagcagaagcctggcggcggcggctctagagtgcgccgcggcggcggcggcagctaccaacagaaacctggcaaggccccaaaactgctgatctacgctgcttctaccctgcagggcggcgggggcagcagagtgagacggggcggcggcggcagcgaggatgtggctacatactactgtcagagatacaaccgggccccttacacttttggccagggcggcggcggcagcagagtgcgtagaggcggaggcggcagctatacctttggccaaggtaccaaggtcgagatcaagcggaccgtggccgcccctagcgtgcgggttcggagatga

SEQ ID NO: 909 - protein, 10 aa, synthetic construct
  REGION 1..10: note = the BDC2.5 1040-51 mimotope
RTRPLWVRME

SEQ ID NO: 910 - protein, 10 aa, synthetic construct
  REGION 1..10: note = BDC2.5 1040-51 mimotope
RVLPLWVRME

SEQ ID NO: 911 - protein, 11 aa, synthetic construct
  REGION 1..11: note = HIP2.5 peptide
LQTLALWSRMD

SEQ ID NO: 912 - protein, 15 aa, Mus musculus
SHLVERLYLVCGERG

SEQ ID NO: 913 - protein, 15 aa, Mus musculus
KKGAAALGIGTDSVI

SEQ ID NO: 914 - protein, 15 aa, Mus musculus
SHLVERLYLVCGEEG

SEQ ID NO: 915 - protein, 15 aa, Mus musculus
SHLVERLYLVCGGEG

SEQ ID NO: 916 - protein, 10 aa, Mus musculus
AVPPLWVRME

SEQ ID NO: 917 - protein, 10 aa, Mus musculus
RVLPLWVRME

SEQ ID NO: 918 - protein, 14 aa, Mus musculus
WSRMDQLAKELTAE

SEQ ID NO: 919 - protein, 11 aa, Mus musculus
LQTLALWSRMD

SEQ ID NO: 920 - protein, 9 aa, Mus musculus
LYLVCGERG

SEQ ID NO: 921 - protein, 9 aa, Mus musculus
VYLKTNVFL

SEQ ID NO: 922 - DNA, 444 bp, synthetic construct
  misc_feature 1..444: note = Codon optimized DNA encodign single epitope BDC2.5)vaccine
  source 1..444: mol_type = other DNA
atgatgatggaccaggcccgaagtgccttcagtaatctatttgggggagagcctctttcctacacacgcttctccctcgccaggcaggtggatggagacaattctcacgtggagatgaagctggctgcagatgaagaagagaatgctgacaacaacatgaaagcaagcgtcaggaaacccaagagattcaacggccgactgtgctttgcagccattgctttagtcatcttttttttgataggcttcatgtctggctatcttgggtactgcaagcgtgttgagcagaaggaggaatgtgtgaaactcgcggaaactgaggaaaccgataagtcagagaccatggagacagaggacgtggggggaggcagccgcgttcggagaggtggaggtggctcgcaacgggtactgccactgtgggtgaggatggaaagggtccgcagatga

SEQ ID NO: 923 - protein, 15 aa, Mus musculus
SHLVERLYLVCGERG

SEQ ID NO: 924 - protein, 15 aa, Mus musculus
KKGAAALGIGTDSVI

SEQ ID NO: 925 - protein, 15 aa, Mus musculus
SHLVERLYLVCGEEG

SEQ ID NO: 926 - protein, 15 aa, Mus musculus
SHLVERLYLVCGGEG

SEQ ID NO: 927 - protein, 10 aa, Mus musculus
AVPPLWVRME

SEQ ID NO: 928 - protein, 10 aa, Mus musculus
RVLPLWVRME

SEQ ID NO: 929 - protein, 14 aa, Mus musculus
WSRMDQLAKELTAE

SEQ ID NO: 930 - protein, 11 aa, Mus musculus
LQTLALWSRMD

SEQ ID NO: 931 - protein, 9 aa, Mus musculus
LYLVCGERG

SEQ ID NO: 932 - protein, 9 aa, Mus musculus
VYLKTNVFL

SEQ ID NO: 933 - DNA, 45 bp, Homo sapiens
  source 1..45: mol_type = other DNA
agccatctggtggaacgcctgtatctggtgtgcggcgaacgcggc

SEQ ID NO: 934 - DNA, 45 bp, Homo sapiens
  source 1..45: mol_type = other DNA
aaaaaaggcgcggcggcgctgggcattggcaccgatagcgtgatt

SEQ ID NO: 935 - DNA, 45 bp, Homo sapiens
  source 1..45: mol_type = other DNA
agccatctggtggaacgcctgtatctggtgtgcggcgaagaaggc

SEQ ID NO: 936 - DNA, 45 bp, Homo sapiens
  source 1..45: mol_type = other DNA
agccatctggtggaacgcctgtatctggtgtgcggcggcgaaggc

SEQ ID NO: 937 - DNA, 30 bp, Homo sapiens
  source 1..30: mol_type = other DNA
gcggtgccgccgctgtgggtgcgcatggaa

SEQ ID NO: 938 - DNA, 30 bp, Homo sapiens
  source 1..30: mol_type = other DNA
cgcgtgctgccgctgtgggtgcgcatggaa

SEQ ID NO: 939 - DNA, 42 bp, Homo sapiens
  source 1..42: mol_type = other DNA
tggagccgcatggatcagctggcgaaagaactgaccgcggaa

SEQ ID NO: 940 - DNA, 33 bp, Homo sapiens
  source 1..33: mol_type = other DNA
ctgcagaccctggcgctgtggagccgcatggat

SEQ ID NO: 941 - DNA, 27 bp, Homo sapiens
  source 1..27: mol_type = other DNA
ctgtatctggtgtgcggcgaacgcggc

SEQ ID NO: 942 - DNA, 27 bp, Homo sapiens
  source 1..27: mol_type = other DNA
gtgtatctgaaaaccaacgtgtttctg

SEQ ID NO: 943 - DNA, 1119 bp, synthetic construct
  misc_feature 1..1119: note = Codon optimized DNA encoding multiple Diabetes T cell epitopes
  source 1..1119: mol_type = other DNA
atgatgatggaccaggccagatcagccttctccaatctctttggcggagagcctcttagctacaccagattttcgttggcgcggcaagtggatggagacaactcccatgtcgagatgaaactggccgctgatgaggaggaaaatgcagataacaacatgaaggcctctgtgcgcaaacccaagcgcttcaacggccggctctgctttgctgccattgcccttgtcatcttcttcctcatcggcttcatgtctggctacctagggtactgcaaaagagttgagcagaaagaagagtgcgtgaagctggcagaaactgaagaaacagacaagtcagagaccatggagacagaagatgttggaggcggctctcgagtacggcgtggaggaggtggctctcagagtcatttagtcgaaaggctgtacttggtgtgtggagaacgggggggcggaggttctcgggtgagaaggggaggcgggggcagcaagaagaaaggagcagctgctctgggcattggaacggacagtgtgataggcgggggaagtcgagtccggagaggaggtgggggcagtgacagccaccttgtagagaggctctatcttgtgtgcggcgaggagggcggaggaggctcgcgtgttcgaaggggaggcggtggcagccagtcccacctggtggaacgcctgtatttagtctgtggcggggaaggcggtggaggctcacgggtccgccgtggaggagggggatcccaagctgtgccaccactctgggtcagaatggaaggtggggggtccagagtcaggcgaggtgggggcggcagccagagggtgctgcccctgtgggtgaggatggaggggggtgggtcaagagttcgacgtgggggaggtggctctcagtggtctcgcatggatcaattggcgaaggagctgactgcagagggaggaggcagcagggtaaggcgcgggggcggtgggtcacagctgcagacactggccctatggagccggatggacctctacctagtatgtggggagagaggtgggggggggtcccgggttaggaggggtgggggaggttcccctgtgtatttgaagaccaatgtgtttctgagagtgcgccgatga

SEQ ID NO: 944 - protein, 9 aa, Homo sapiens
QPLALEGSL

SEQ ID NO: 945 - protein, 7 aa, Homo sapiens
LALEGSL

SEQ ID NO: 946 - protein, 12 aa, Homo sapiens
SLQPLALEGSLQ

SEQ ID NO: 947 - protein, 7 aa, Homo sapiens
LALEGSL

SEQ ID NO: 948 - protein, 20 aa, Homo sapiens
YEMVFDGKPQHTNVCFWYIP

SEQ ID NO: 949 - protein, 24 aa, Homo sapiens
MALWMRLLPLLALLALWGPDPAAA

SEQ ID NO: 950 - protein, 20 aa, Homo sapiens
LALWGPDPAAAFVNQHLCGS

SEQ ID NO: 951 - protein, 20 aa, Homo sapiens
HLVEALYLVCGERGFFYTPK

SEQ ID NO: 952 - protein, 20 aa, Homo sapiens
GERGFFYTPKTRREAEDLQV

SEQ ID NO: 953 - protein, 20 aa, Homo sapiens
GQVELGGGPGAGSLQPLALE

SEQ ID NO: 954 - protein, 20 aa, Homo sapiens
AGSLQPLALEGSLQKRGIVE

SEQ ID NO: 955 - protein, 17 aa, Homo sapiens
QCCTSICSLYQLENYCN

SEQ ID NO: 956 - protein, 14 aa, Homo sapiens
WSKMDQLAKELTAE

SEQ ID NO: 957 - protein, 20 aa, Homo sapiens
YVVKSFDRSTKVIDFHYPNE

SEQ ID NO: 958 - protein, 20 aa, Homo sapiens
NTNMFTYEIAPVFVLLEYVT

SEQ ID NO: 959 - protein, 20 aa, Homo sapiens
PGGSGDGIFSPGGAISNMYA

SEQ ID NO: 960 - protein, 17 aa, Homo sapiens
AMMIARFKMFPEVKEKG

SEQ ID NO: 961 - protein, 20 aa, Homo sapiens
EVKEKGMAALPRLIAFTSEH

SEQ ID NO: 962 - protein, 20 aa, Homo sapiens
VDKCLELAEYLYNIIKNREG

SEQ ID NO: 963 - protein, 15 aa, Homo sapiens
CFWYIPPSLRTLEDN

SEQ ID NO: 964 - protein, 15 aa, Homo sapiens
ERMSRLSKVAPVIKA

SEQ ID NO: 965 - protein, 15 aa, Homo sapiens
RMMEYGTTMVSYQPL

SEQ ID NO: 966 - protein, 19 aa, Homo sapiens
SLSYEPALLEPYLFHEFGS

SEQ ID NO: 967 - protein, 16 aa, Homo sapiens
AAEEYGYIVTDEKPLS

SEQ ID NO: 968 - protein, 14 aa, Homo sapiens
QNLSLADVTEEAGL

SEQ ID NO: 969 - protein, 12 aa, Homo sapiens
TGLEILETGVGE

SEQ ID NO: 970 - protein, 6 aa, Synthetic construct
REEAAA

SEQ ID NO: 971 - protein, 18 aa, Homo sapiens
RQHARQQDKERLAALGPE

SEQ ID NO: 972 - protein, 18 aa, Homo sapiens
GPEGAHGDTTFEYQDLCR

SEQ ID NO: 973 - protein, 18 aa, Homo sapiens
EGPPEPSRVSSVSSQFSD

SEQ ID NO: 974 - protein, 18 aa, Homo sapiens
FSDAAQASPSSHSSTPSW

SEQ ID NO: 975 - protein, 18 aa, Homo sapiens
AEPNTCATAQGEGNIKKN

SEQ ID NO: 976 - protein, 18 aa, Homo sapiens
NASPIIEHDPRMPAYIAT

SEQ ID NO: 977 - protein, 18 aa, Homo sapiens
DEGASLYHVYEVNLVSEH

SEQ ID NO: 978 - protein, 18 aa, Homo sapiens
KGVKEIDIAATLEHVRDQ

SEQ ID NO: 979 - protein, 19 aa, Homo sapiens
FALTAVAEEVNAILKALPQ

SEQ ID NO: 980 - protein, 20 aa, Homo sapiens
  SITE 9: note = misc_feature - Xaa is citrulline
  SITE 17: note = misc_feature - Xaa is citrulline
VGSNTYGKXNAVEVLKXEPL

SEQ ID NO: 981 - protein, 24 aa, Homo sapiens
MALWMRLLPLLALLALWGPDPAAA

SEQ ID NO: 982 - protein, 16 aa, Homo sapiens
LPLLALLALWGPDPAA

SEQ ID NO: 983 - protein, 22 aa, Homo sapiens
LALWGPDPAAAFVNQHLCGSHL

SEQ ID NO: 984 - protein, 16 aa, Homo sapiens
FVNQHLCGSHLVEALY

SEQ ID NO: 985 - protein, 24 aa, Homo sapiens
LCGSHLVEALYLVCGERGFFYTPK

SEQ ID NO: 986 - protein, 16 aa, Homo sapiens
YLVCGERGFFYTPKTR

SEQ ID NO: 987 - protein, 20 aa, Homo sapiens
GERGFFYTPKTRREAEDLQV

SEQ ID NO: 988 - protein, 16 aa, Homo sapiens
RREAEDLQVGQVELGG

SEQ ID NO: 989 - protein, 10 aa, Homo sapiens
AEDLQVGQVE

SEQ ID NO: 990 - protein, 16 aa, Homo sapiens
LQVGQVELGGGPGAGS

SEQ ID NO: 991 - protein, 9 aa, Homo sapiens
VELGGGPGA

SEQ ID NO: 992 - protein, 24 aa, Homo sapiens
GGPGAGSLQPLALEGSLQKRGIVE

SEQ ID NO: 993 - protein, 24 aa, Homo sapiens
AGSLQPLALEGSLQKRGIVEQCCT

SEQ ID NO: 994 - protein, 19 aa, Homo sapiens
VEQCCTSICSLYQLENYCN

SEQ ID NO: 995 - protein, 15 aa, Homo sapiens
GQVELGGGSSPETLI

SEQ ID NO: 996 - protein, 18 aa, Homo sapiens
LQDIPTGSAPAAQHRLPQ

SEQ ID NO: 997 - protein, 8 aa, Homo sapiens
WGPDPAAA

SEQ ID NO: 998 - protein, 15 aa, Homo sapiens
SHLVEALYLVCGERG

SEQ ID NO: 999 - protein, 18 aa, Homo sapiens
PGAGSLQPLALEGSLQKR

SEQ ID NO: 1000 - protein, 11 aa, Homo sapiens
SLQPLALEGSL

SEQ ID NO: 1001 - protein, 12 aa, Homo sapiens
NYAFLHATDLLP

SEQ ID NO: 1002 - protein, 12 aa, Homo sapiens
MYAMMIARFKMF

SEQ ID NO: 1003 - protein, 19 aa, Homo sapiens
GLMQNCNQMHASYLFQQDK

SEQ ID NO: 1004 - protein, 20 aa, Homo sapiens
HTNVCFWYIPPSLRTLEDNE

SEQ ID NO: 1005 - protein, 20 aa, Homo sapiens
NMYAMMIARFKMFPEVKEKG

SEQ ID NO: 1006 - protein, 18 aa, Homo sapiens
TAGTTVYGAFDPLLAVAD

SEQ ID NO: 1007 - protein, 20 aa, Homo sapiens
YEMVFDGKPQHTNVCFWYIP

SEQ ID NO: 1008 - protein, 20 aa, Homo sapiens
PSLRTLEDNEERMSRLSKVA

SEQ ID NO: 1009 - protein, 24 aa, Homo sapiens
DRGEKPASPAVQPDAALQRLAAVL

SEQ ID NO: 1010 - protein, 40 aa, Homo sapiens
SPLGQSQPTVAGQPSARPAAEEYGYIVTDQKPLSLAAGVK

SEQ ID NO: 1011 - protein, 13 aa, Homo sapiens
QHLQKDYRAYYTF

SEQ ID NO: 1012 - protein, 13 aa, Homo sapiens
RVLNIDLLWSVPI

SEQ ID NO: 1013 - protein, 13 aa, Homo sapiens
FFYTPKTRREAED

SEQ ID NO: 1014 - protein, 27 aa, Homo sapiens
SLWLSSKPPSKRLTFGWHRAEILGALL

SEQ ID NO: 1015 - protein, 41 aa, Homo sapiens
FGWHRAEILGALLSILCIWVVTGVLVYLACERLLYPDYQIQ

SEQ ID NO: 1016 - protein, 34 aa, Homo sapiens
ASRDSQVVRREIAKALSKSFTMHSLTIQMESPVD

SEQ ID NO: 1017 - protein, 20 aa, Homo sapiens
RLQQLVLDPGFLGLEPLLDL

SEQ ID NO: 1018 - protein, 20 aa, Homo sapiens
MASPGSGFWSFGSEDGSGDS

SEQ ID NO: 1019 - protein, 20 aa, Homo sapiens
CACDQKPCSCSKVDVNYAFL

SEQ ID NO: 1020 - protein, 20 aa, Homo sapiens
  SITE 17: note = misc_feature - Xaa is citrulline
YAFLHATDLLPACDGEXPTL

SEQ ID NO: 1021 - protein, 20 aa, Homo sapiens
RPTLAFLQDVMNILLQYVVK

SEQ ID NO: 1022 - protein, 20 aa, Homo sapiens
DVMNILLQYVVKSFDRSTKV

SEQ ID NO: 1023 - protein, 13 aa, Homo sapiens
MNILLEYVVKSFD

SEQ ID NO: 1024 - protein, 20 aa, Homo sapiens
PQNLEEILMHCQTTLKYAIK

SEQ ID NO: 1025 - protein, 12 aa, Homo sapiens
GHPRYFNQLSTG

SEQ ID NO: 1026 - protein, 20 aa, Homo sapiens
NTNMFTYEIAPVFVLLEYVT

SEQ ID NO: 1027 - protein, 23 aa, Homo sapiens
KGMAALPRLIAFTSEHSHFSLKK

SEQ ID NO: 1028 - protein, 20 aa, Homo sapiens
  SITE 8: note = misc_feature - Xaa is citrulline
KGMAALPXLIAFTSEHSHFS

SEQ ID NO: 1029 - protein, 20 aa, Homo sapiens
LIAFTSEHSHFSLKKGAAAL

SEQ ID NO: 1030 - protein, 20 aa, Homo sapiens
DSVILIKCDERGKMIPSDLE

SEQ ID NO: 1031 - protein, 20 aa, Homo sapiens
DERGKMIPSDLERRILEAKQ

SEQ ID NO: 1032 - protein, 20 aa, Homo sapiens
EAKQKGFVPFLVSATAGTTV

SEQ ID NO: 1033 - protein, 15 aa, Homo sapiens
KYKIWMHVDAAWGGG

SEQ ID NO: 1034 - protein, 21 aa, Homo sapiens
KHKWKLSGVERANSVTWNPHK

SEQ ID NO: 1035 - protein, 20 aa, Homo sapiens
YDLSYDTGDKALQCGRHVDV

SEQ ID NO: 1036 - protein, 23 aa, Homo sapiens
KCLELAEYLYNIIKNREGYEMVF

SEQ ID NO: 1037 - protein, 15 aa, Homo sapiens
PSLRTLEDNEERMSR

SEQ ID NO: 1038 - protein, 20 aa, Homo sapiens
VSYQPLGDKVNFFRMVISNP

SEQ ID NO: 1039 - protein, 25 aa, Homo sapiens
GDKVNFFRMVISNPAATHQDIDFLI

SEQ ID NO: 1040 - protein, 23 aa, Homo sapiens
  SITE 6: note = misc_feature - Xaa is citrulline
KVNFFXMVISNPAATHQDIDFLI

SEQ ID NO: 1041 - protein, 16 aa, Homo sapiens
AATHQDIDFLIEEIER

SEQ ID NO: 1042 - protein, 15 aa, Homo sapiens
VMRIINEPTAAAIAY

SEQ ID NO: 1043 - protein, 15 aa, Homo sapiens
  SITE 3: note = misc_feature - Xaa is citrulline
VMXIINEPTAAAIAY

SEQ ID NO: 1044 - protein, 15 aa, Homo sapiens
EVTFEIDVNGILRVT

SEQ ID NO: 1045 - protein, 17 aa, Homo sapiens
  SITE 13: note = misc_feature - Xaa is citrulline
EVTFEIDVNGILXVTAE

SEQ ID NO: 1046 - protein, 18 aa, Homo sapiens
VSSQFSDAAQASPSSHSS

SEQ ID NO: 1047 - protein, 25 aa, Homo sapiens
LAKEWQALCAYQAEPNTCATAQGEG

SEQ ID NO: 1048 - protein, 20 aa, Homo sapiens
IKLKVESSPSRSDYINASPI

SEQ ID NO: 1049 - protein, 21 aa, Homo sapiens
MVWESGCTVIVMLTPLVEDGV

SEQ ID NO: 1050 - protein, 19 aa, Homo sapiens
FYLKNVQTQETRTLTQFHF

SEQ ID NO: 1051 - protein, 21 aa, Homo sapiens
SKDQFEFALTAVAEEVNAILK

SEQ ID NO: 1052 - protein, 20 aa, Homo sapiens
KDYRAYYTFLNFMSNVGDPR

SEQ ID NO: 1053 - protein, 13 aa, Homo sapiens
YTFLNFMSNVGDP

SEQ ID NO: 1054 - protein, 20 aa, Homo sapiens
KWCANPDWIHIDTTPFAGLV

SEQ ID NO: 1055 - protein, 16 aa, Homo sapiens
LALLALWGPDPAAAFV

SEQ ID NO: 1056 - protein, 16 aa, Homo sapiens
PAAAFVNQHLCGSHLV

SEQ ID NO: 1057 - protein, 26 aa, Homo sapiens
GGGPGAGSLQPLALEGSLQKRGIVEQ

SEQ ID NO: 1058 - protein, 11 aa, Homo sapiens
SLQPLALEGSL

SEQ ID NO: 1059 - protein, 10 aa, Homo sapiens
QPLALEGSLQ

SEQ ID NO: 1060 - protein, 9 aa, Homo sapiens
LALEGSLQK

SEQ ID NO: 1061 - protein, 20 aa, Homo sapiens
SLQKRGIVEQCCTSICSLYQ

SEQ ID NO: 1062 - protein, 20 aa, Homo sapiens
KAMVALIDVFHQYSGREGDK

SEQ ID NO: 1063 - protein, 16 aa, Homo sapiens
REGDKHKLKKSELKEL

SEQ ID NO: 1064 - protein, 22 aa, Homo sapiens
KHKLKKSELKELINNELSHFLE

SEQ ID NO: 1065 - protein, 25 aa, Homo sapiens
ECDFQEFMAFVAMVTTACHEFFEHE

SEQ ID NO: 1066 - protein, 13 aa, Homo sapiens
SILLMEGVPKSLN

SEQ ID NO: 1067 - protein, 15 aa, Homo sapiens
MIARFKMFPEVKEKG

SEQ ID NO: 1068 - protein, 9 aa, Homo sapiens
FTSEHSHFS

SEQ ID NO: 1069 - protein, 13 aa, Homo sapiens
NFFRMVISNPAAT

SEQ ID NO: 1070 - protein, 20 aa, Homo sapiens
MMIARFKMFPEVKEKGMAAL

SEQ ID NO: 1071 - protein, 20 aa, Homo sapiens
NWELADQPQNLEEILMHCQT

SEQ ID NO: 1072 - protein, 17 aa, Homo sapiens
LVEALYLVCGERGFFYT

SEQ ID NO: 1073 - protein, 20 aa, Homo sapiens
DVMNILLQYVVKSFDRSTKV

SEQ ID NO: 1074 - protein, 20 aa, Homo sapiens
IAPVFVLLEYVTLKKMREII

SEQ ID NO: 1075 - protein, 15 aa, Homo sapiens
LPRLIAFTSEHSHFS

SEQ ID NO: 1076 - protein, 20 aa, Homo sapiens
ICKKYKIWMHVDAAWGGGLL

SEQ ID NO: 1077 - protein, 20 aa, Homo sapiens
VAPVIKARMMEYGTTMVSYQ

SEQ ID NO: 1078 - protein, 20 aa, Homo sapiens
KVNFFRMVISNPAATHQDID

SEQ ID NO: 1079 - protein, 13 aa, Homo sapiens
NPAATHQDIDFLI

SEQ ID NO: 1080 - protein, 20 aa, Homo sapiens
ATHQDIDFLIEEIERLGQDL

SEQ ID NO: 1081 - protein, 20 aa, Homo sapiens
PLLALLALWGPDPAAAFVNQ

SEQ ID NO: 1082 - protein, 12 aa, Homo sapiens
NYAFLHATDLLP

SEQ ID NO: 1083 - protein, 10 aa, Homo sapiens
MYAMMIARFK

SEQ ID NO: 1084 - protein, 21 aa, Homo sapiens
GGGLLMSRKHKWKLSGVERAN

SEQ ID NO: 1085 - protein, 20 aa, Homo sapiens
LYHVYEVNLVSEHIWCEDFL

SEQ ID NO: 1086 - protein, 20 aa, Homo sapiens
SEHIWCEDFLVRSFYLKNVQ

SEQ ID NO: 1087 - protein, 13 aa, Homo sapiens
MNILLQYVVKSFD

SEQ ID NO: 1088 - protein, 13 aa, Homo sapiens
IAFTSEHSHFSLK

SEQ ID NO: 1089 - protein, 15 aa, Homo sapiens
SHLVEALYLVCGERG

SEQ ID NO: 1090 - DNA, 39 bp, Homo sapiens
  source 1..39: mol_type = other DNA
atgaacattctgctgcagtatgtggtgaaaagctttgat

SEQ ID NO: 1091 - DNA, 39 bp, Homo sapiens
  source 1..39: mol_type = other DNA
attgcgtttaccagcgaacatagccattttagcctgaaa

SEQ ID NO: 1092 - DNA, 45 bp, Homo sapiens
  source 1..45: mol_type = other DNA
agccatctggtggaagcgctgtatctggtgtgcggcgaacgcggc

SEQ ID NO: 1093 - DNA, 621 bp, synthetic construct
  misc_feature 1..621: note = Codon optimized DNA encoding multi-epitope vaccine for use in humans with tyoe tyoe I diabetes and displaying a DRB1*0401 genotype
  source 1..621: mol_type = other DNA
atgatggaccaggccagaagcgcctttagcaacctgttcggcggagagcctctgagctacaccagattcagcctggctagacaggtggatggcgacaatagccacgtggaaatgaaactggccgtggacgaggaagagaacgccgataacaacaccaaggccaatgtgacaaaacctaagcggtgcagcggctccatctgctacggcaccatcgccgtgatcgtgttcttcctgatcggctttatgatcggctatctgggctactgcaagggcgtggaacccaagaccgagtgtgaaagactggctggcacagagagccctgtgcgggaagaaccaggcgaggacttccccgccggcggaggcggcggctccagagtgcggagaggcggcggcggctctatgaacatcctgctgcagtacgtggtgaagagcttcgacggcggcggcggctctagagtgcgcagaggcggcggcggatctattgccttcaccagcgagcacagccatttcagcctgaagggcggaggcggatctagggttagacggggaggaggaggttccagccacctggtcgaggccctgtacctcgtgtgcggcgagagaggcagagtccggcggtga

SEQ ID NO: 1094 - protein, 22 aa, Homo sapiens
RIHMVYSKRPSGKPRGYAFIEY

SEQ ID NO: 1095 - protein, 21 aa, Homo sapiens
STDHPKYSDMIVAAIQAEKNR

SEQ ID NO: 1096 - protein, 24 aa, Homo sapiens
DLRFQSSAVMALQEACEAYLVGLF

SEQ ID NO: 1097 - protein, 21 aa, Homo sapiens
KRVTIMPKDIQLARRIRGERA

SEQ ID NO: 1098 - protein, 17 aa, Homo sapiens
KRHRKVLRDNIQGITKP

SEQ ID NO: 1099 - protein, 23 aa, Homo sapiens
TYTEHAKRKTVAMDVVYALKRQG

SEQ ID NO: 1100 - protein, 15 aa, Homo sapiens
EIAEEYGGVMVSFPR

SEQ ID NO: 1101 - DNA, 63 bp, Homo sapiens
  source 1..63: mol_type = other DNA
agcaccgatcatccgaaatatagcgatatgattgtggcggcgattcaggcggaaaaaaaccgc

SEQ ID NO: 1102 - DNA, 72 bp, Homo sapiens
  source 1..72: mol_type = other DNA
gatctgcgctttcagagcagcgcggtgatggcgctgcaggaagcgtgcgaagcgtatctggtgggcctgttt

SEQ ID NO: 1103 - DNA, 63 bp, Homo sapiens
  source 1..63: mol_type = other DNA
aaacgcgtgaccattatgccgaaagatattcagctggcgcgccgcattcgcggcgaacgcgcg

SEQ ID NO: 1104 - DNA, 51 bp, Homo sapiens
  source 1..51: mol_type = other DNA
aaacgccatcgcaaagtgctgcgcgataacattcagggcattaccaaaccg

SEQ ID NO: 1105 - DNA, 69 bp, Homo sapiens
  source 1..69: mol_type = other DNA
acctataccgaacatgcgaaacgcaaaaccgtggcgatggatgtggtgtatgcgctgaaacgccagggc

SEQ ID NO: 1106 - DNA, 45 bp, Homo sapiens
  source 1..45: mol_type = other DNA
gaaattgcggaagaatatggcggcgtgatggtgagctttccgcgc

SEQ ID NO: 1107 - DNA, 483 bp, synthetic construct
  misc_feature 1..483: note = Codon-optimized DNA encodign control peptide
  source 1..483: mol_type = other DNA
atgatgatggaccaggcacggagtgccttcagcaatctctttggaggggagcctctgtcctatactcgcttctctttagcacggcaggtggatggagacaacagccatgttgagatgaagcttgcagcagatgaggaagaaaatgctgataacaacatgaaagccagtgtacgtaaaccaaaaaggtttaatggccgcctctgctttgctgccatagcgttggtcatctttttcctgattggcttcatgtctggctacctaggatactgcaagagagtggagcagaaggaagaatgtgtgaagctggctgagaccgaggagacagacaaatcagaaaccatggagactgaagacgtaggtgggggctccagagtcagaagaggtggcggagggtcacagacctacacagagcacgccaagaggaaaacggttgccatggatgtggtgtatgctctgaagcggcaagggcgagtcaggcgatga

SEQ ID NO: 1108 - DNA, 483 bp, synthetic construct; identical to SEQ ID NO: 1107
  misc_feature 1..483: note = Codon optimized H4 71-94 cDNA sequence
  source 1..483: mol_type = other DNA

SEQ ID NO: 1109 - DNA, 900 bp, synthetic construct
  misc_feature 1..900: note = Codon-optimized DN encoding multiple histone auto-epitopes
  source 1..900: mol_type = other DNA
atgatgatggatcaggcccgatcagccttcagcaacctctttggaggcgagcctctatcttatactagattttctttagctaggcaagtggatggagacaattctcatgtcgagatgaaacttgcagcagatgaggaagagaatgcagacaacaacatgaaggcctcagtgagaaaaccaaaaaggttcaatggccgcctctgctttgctgcaattgctctggtcatcttctttctgataggcttcatgagtggctatctgggttactgcaaacgagtagagcagaaggaagaatgtgtgaaacttgcagaaactgaggagactgacaaatcagagaccatggagacagaagacgttggaggagggagcagagttcgccgcgggggaggaggaagccagagcacagaccaccccaagtacagtgacatgattgtggccgctatccaagcagagaagaatagaggcggcggctccagggtccgcaggggcggcggtgggtccaaggacctgcgtttccagtcctctgctgtgatggcgctgcaggaggcctgtgaagcgtacttggtgggcttgtttgggggcggttcacgcgtcagaaggggtgggggaggttccgacaagcgggtcaccatcatgccaaaggatattcagctggcccggaggatacggggagaaagggctggtgggggtagtcgtgtacgtagagggggcggaggctctgtcaagcgacacaggaaggtgctccgggataacatccagggcatcaccaagcctgggggtggaagcagagtacgcaggggggggggaggaagccccacctacacggaacatgccaagcggaaaacagttgccatggatgtggtgtatgctttgaaaagacaagggagagtgcggcgctga

SEQ ID NO: 1110 - protein, 9 aa, Arachis hypogaea
VPQYGYLTL

SEQ ID NO: 1111 - protein, 9 aa, Arachis hypogaea
QPARKRLNF

SEQ ID NO: 1112 - protein, 8 aa, Arachis hypogaea
PQYGYLTL

SEQ ID NO: 1113 - protein, 8 aa, Arachis hypogaea
FSYTFEDV

SEQ ID NO: 1114 - protein, 8 aa, Arachis hypogaea
MVPQYGYL

SEQ ID NO: 1115 - protein, 9 aa, Arachis hypogaea
QYGSVSTNL

SEQ ID NO: 1116 - protein, 9 aa, Arachis hypogaea
QYSTGQVSV

SEQ ID NO: 1117 - protein, 9 aa, Arachis hypogaea
VFTDSEYQL

SEQ ID NO: 1118 - protein, 8 aa, Arachis hypogaea
SEKTNVDI

SEQ ID NO: 1119 - protein, 8 aa, Arachis hypogaea
SEYQLPYV

SEQ ID NO: 1120 - protein, 8 aa, Arachis hypogaea
VDIEKVMI

SEQ ID NO: 1121 - protein, 9 aa, Arachis hypogaea
LDRLMNPLI

SEQ ID NO: 1122 - protein, 9 aa, Arachis hypogaea
EEIRTTNPV

SEQ ID NO: 1123 - protein, 9 aa, Arachis hypogaea
RDVYLQGPI

SEQ ID NO: 1124 - protein, 9 aa, Arachis hypogaea
TADVNTQGV

SEQ ID NO: 1125 - protein, 9 aa, Arachis hypogaea
FSPRDWQRL

SEQ ID NO: 1126 - protein, 9 aa, Arachis hypogaea
VMITDEEEI

SEQ ID NO: 1127 - protein, 9 aa, Arachis hypogaea
VPQYGYLTL

SEQ ID NO: 1128 - protein, 9 aa, Arachis hypogaea
FPQSGCLIF

SEQ ID NO: 1129 - protein, 9 aa, Arachis hypogaea
SPLMGGFGL

SEQ ID NO: 1130 - protein, 8 aa, Arachis hypogaea
PQYGYLTL

SEQ ID NO: 1131 - protein, 8 aa, Arachis hypogaea
FTYTFEDV

SEQ ID NO: 1132 - protein, 8 aa, Arachis hypogaea
MIPQYGYL

SEQ ID NO: 1133 - protein, 9 aa, Arachis hypogaea
KYLGPFNGL

SEQ ID NO: 1134 - protein, 10 aa, Arachis hypogaea
EYGIVADNLD

SEQ ID NO: 1135 - protein, 9 aa, Arachis hypogaea
VFTDSEYQL

SEQ ID NO: 1136 - protein, 8 aa, Arachis hypogaea
EDNLSEGI

SEQ ID NO: 1137 - protein, 8 aa, Arachis hypogaea
QDDGRGLV

SEQ ID NO: 1138 - protein, 8 aa, Arachis hypogaea
SEYQLPYV

SEQ ID NO: 1139 - protein, 9 aa, Arachis hypogaea
LEDNLSEGI

SEQ ID NO: 1140 - protein, 9 aa, Arachis hypogaea
LDRLMNPLI

SEQ ID NO: 1141 - protein, 9 aa, Arachis hypogaea
EEIKTTNPV

SEQ ID NO: 1142 - protein, 9 aa, Arachis hypogaea
FFPSNGILI

SEQ ID NO: 1143 - protein, 9 aa, Arachis hypogaea
GGTANTQTL

SEQ ID NO: 1144 - protein, 9 aa, Arachis hypogaea
TGQNNNSNF

SEQ ID NO: 1145 - protein, 9 aa, Arachis hypogaea
QPARKRLNF

SEQ ID NO: 1146 - protein, 9 aa, Arachis hypogaea
LPPFPADVF

SEQ ID NO: 1147 - protein, 9 aa, Arachis hypogaea
FPSNGILIF

SEQ ID NO: 1148 - protein, 8 aa, Arachis hypogaea
ADKEYTNI

SEQ ID NO: 1149 - protein, 8 aa, Arachis hypogaea
SFNDFTRV

SEQ ID NO: 1150 - protein, 8 aa, Arachis hypogaea
KYGIYTKV

SEQ ID NO: 1151 - protein, 9 aa, Arachis hypogaea
KYNHDIALL

SEQ ID NO: 1152 - protein, 10 aa, Arachis hypogaea
GYVSGWGRVD

SEQ ID NO: 1153 - protein, 9 aa, Arachis hypogaea
SYVTPICIA

SEQ ID NO: 1154 - protein, 8 aa, Arachis hypogaea
AETILDNI

SEQ ID NO: 1155 - protein, 8 aa, Arachis hypogaea
TEQKRNVI

SEQ ID NO: 1156 - protein, 8 aa, Arachis hypogaea
ADKEYTNI

SEQ ID NO: 1157 - protein, 9 aa, Arachis hypogaea
CELDVTCNI

SEQ ID NO: 1158 - protein, 9 aa, Arachis hypogaea
AESPGLITI

SEQ ID NO: 1159 - protein, 9 aa, Arachis hypogaea
VDAFCGGSI

SEQ ID NO: 1160 - protein, 10 aa, Arachis hypogaea
GSIVNEKWID

SEQ ID NO: 1161 - protein, 9 aa, Arachis hypogaea
LDHENANKI

SEQ ID NO: 1162 - protein, 9 aa, Arachis hypogaea
EQKRNVIRI

SEQ ID NO: 1163 - protein, 9 aa, Arachis hypogaea
SPGLITICL

SEQ ID NO: 1164 - protein, 9 aa, Arachis hypogaea
RSALVLQYL

SEQ ID NO: 1165 - protein, 9 aa, Arachis hypogaea
NSYECWCPF

SEQ ID NO: 1166 - protein, 15 aa, Arachis hypogaea
LEPLGLVEEGAKTAP

SEQ ID NO: 1167 - protein, 15 aa, Arachis hypogaea
QVSVEIEWELQKENS

SEQ ID NO: 1168 - protein, 15 aa, Arachis hypogaea
NSYVTPICIADKEYT

SEQ ID NO: 1169 - protein, 15 aa, Arachis hypogaea
YTKVSRYVNWIKEKT

SEQ ID NO: 1170 - protein, 15 aa, Arachis hypogaea
QVSVEIEWELQKENS

SEQ ID NO: 1171 - protein, 14 aa, Arachis hypogaea
GVLIFGKQSEKTNV

SEQ ID NO: 1172 - protein, 15 aa, Arachis hypogaea
TSADNNNSEYSWTGA

SEQ ID NO: 1173 - protein, 15 aa, Arachis hypogaea
NGRDSLVNPGPAMAS

SEQ ID NO: 1174 - protein, 15 aa, Arachis hypogaea
LEPLGLVEEGAKTAP

SEQ ID NO: 1175 - protein, 16 aa, Arachis hypogaea
YKYLLGPFNGLDKGEP

SEQ ID NO: 1176 - protein, 15 aa, Arachis hypogaea
IVADNLQQQNTAPQI

SEQ ID NO: 1177 - protein, 15 aa, Arachis hypogaea
DNLSEGIREWWALKP

SEQ ID NO: 1178 - protein, 16 aa, Arachis hypogaea
NSYVTPICIADEKEYT

SEQ ID NO: 1179 - protein, 16 aa, Arachis hypogaea
YTKVSRYVNWIEKEKT

SEQ ID NO: 1180 - protein, 14 aa, Arachis hypogaea
NKWCSCTEGYRLAE

SEQ ID NO: 1181 - protein, 17 aa, Arachis hypogaea
YNHDIALLELLEDEPLV

SEQ ID NO: 1182 - protein, 15 aa, Arachis hypogaea
CKDDINSYECWCPFG

SEQ ID NO: 1183 - protein, 15 aa, Arachis hypogaea
QYVDGDQCESNPCLN

SEQ ID NO: 1184 - protein, 9 aa, Arachis hypogaea
QYGSVSTNL

SEQ ID NO: 1185 - protein, 9 aa, Arachis hypogaea
KYLGPFNGL

SEQ ID NO: 1186 - protein, 9 aa, Arachis hypogaea
KYNHDIALL

SEQ ID NO: 1187 - protein, 15 aa, Arachis hypogaea
QVSVEIEWELQKENS

SEQ ID NO: 1188 - protein, 15 aa, Arachis hypogaea
LEPLGLVEEGAKTAP

SEQ ID NO: 1189 - protein, 15 aa, Arachis hypogaea
CKDDINSYECWCPFG

SEQ ID NO: 1190 - DNA, 27 bp, Arachis hypogaea
  source 1..27: mol_type = other DNA
cagtatggcagcgtgagcaccaacctg

SEQ ID NO: 1191 - DNA, 27 bp, Arachis hypogaea
  source 1..27: mol_type = other DNA
aaatatctgggcccgtttaacggcctg

SEQ ID NO: 1192 - DNA, 27 bp, Arachis hypogaea
  source 1..27: mol_type = other DNA
aaatataaccatgatattgcgctgctg

SEQ ID NO: 1193 - DNA, 45 bp, Arachis hypogaea
  source 1..45: mol_type = other DNA
caggtgagcgtggaaattgaatgggaactgcagaaagaaaacagc

SEQ ID NO: 1194 - DNA, 45 bp, Arachis hypogaea
  source 1..45: mol_type = other DNA
ctggaaccgctgggcctggtggaagaaggcgcgaaaaccgcgccg

SEQ ID NO: 1195 - DNA, 45 bp, Arachis hypogaea
  source 1..45: mol_type = other DNA
tgcaaagatgatattaacagctatgaatgctggtgcccgtttggc

SEQ ID NO: 1196 - DNA, 840 bp, synthetic construct
  misc_feature 1..840: note = Codon-optimized sequence for multi-epitope delivery of tolerogenic AAV epitopes.
  source 1..840: mol_type = other DNA
atgatgatggaccaggccaggtccgccttttctaatctctttggcggagagcccttaagctacacacgcttctccctggcgagacaagtggatggagataatagtcacgtggagatgaagcttgctgcagatgaagaagagaatgctgacaacaacatgaaagcaagtgtcagaaaaccaaagaggtttaatggacgtctttgttttgctgccatcgctctggtgatcttcttcctgattggcttcatgtctggctatctcggctactgtaaacgagtggagcagaaggaggagtgtgtgaagctggcagagacggaggaaactgacaagtcagagaccatggagacagaagatgtgggtggaggaagtcgggttcgccggggagggggtggtagccagcagtatggctctgtctccaccaacctggggggcggaggatcccgagtcaggagaggcgggggggggagcaaataccttggtcctttcaatggcttgggtggtggagggtcacgggtccgaaggggcgggggcggcagtaagtacaaccatgacatagccctattgggcggtggtggctcgcgcgtcaggagaggaggcggcggatcacaagtttctgtggaaattgagtgggagctgcagaaagaaaacagcgggggaggaggctccagagtacggagaggggggggggggtcgctggagcctctagggctcgtagaagaaggagccaagactgccccaggaggaggtggcagcagggtgcgccggggcgggggtggtagctgcaaggatgacatcaactcttatgaatgctggtgcccctttgggcgtgttagaaggtga

SEQ ID NO: 1197 - protein, 9 aa, Homo sapiens
RLYRKRGLK

SEQ ID NO: 1198 - protein, 15 aa, Arachis hypogaea
LALFLLAAHASARQQ

SEQ ID NO: 1199 - protein, 15 aa, Arachis hypogaea
LRNLPQQCGLRAPQR

SEQ ID NO: 1200 - protein, 15 aa, Arachis hypogaea
MAKLTILVALALFLL

SEQ ID NO: 1201 - protein, 15 aa, Arachis hypogaea
SYGRDPYSPSQDPYS

SEQ ID NO: 1202 - protein, 10 aa, Arachis hypogaea
HASARQQWEL

SEQ ID NO: 1203 - protein, 15 aa, Arachis hypogaea
QDRQMVQQFKRELMN

SEQ ID NO: 1204 - protein, 15 aa, Arachis hypogaea
SAMRRERGRQGDSSS

SEQ ID NO: 1205 - protein, 15 aa, Arachis hypogaea
QYDSYDIRSTRSSDQ

SEQ ID NO: 1206 - protein, 20 aa, Arachis hypogaea
DSYERDPYSPSQDPYSPSPY

SEQ ID NO: 1207 - protein, 15 aa, Arachis hypogaea
LALFLLAAHASARQQ

SEQ ID NO: 1208 - protein, 15 aa, Arachis hypogaea
LRNLPQQCGLRAPQR

SEQ ID NO: 1209 - protein, 15 aa, Arachis hypogaea
MAKLTILVALALFLL

SEQ ID NO: 1210 - protein, 15 aa, Arachis hypogaea
SYGRDPYSPSQDPYS

SEQ ID NO: 1211 - protein, 15 aa, Arachis hypogaea
QDRQMVQQFKRELMN

SEQ ID NO: 1212 - protein, 15 aa, Arachis hypogaea
SAMRRERGRQGDSSS

SEQ ID NO: 1213 - protein, 15 aa, Arachis hypogaea
QYDSYDIRSTRSSDQ

SEQ ID NO: 1214 - protein, 15 aa, Arachis hypogaea
LALFLLAAHASARQQ

SEQ ID NO: 1215 - DNA, 45 bp, Arachis hypogaea
  source 1..45: mol_type = other DNA
ctggcgctgtttctgctggcggcgcatgcgagcgcgcgccagcag

SEQ ID NO: 1216 - protein, 15 aa, Arachis hypogaea
LRNLPQQCGLRAPQR

SEQ ID NO: 1217 - DNA, 45 bp, Arachis hypogaea
  source 1..45: mol_type = other DNA
ctgcgcaacctgccgcagcagtgcggcctgcgcgcgccgcagcgc

SEQ ID NO: 1218 - protein, 15 aa, Arachis hypogaea
MAKLTILVALALFLL

SEQ ID NO: 1219 - DNA, 45 bp, Arachis hypogaea
  source 1..45: mol_type = other DNA
atggcgaaactgaccattctggtggcgctggcgctgtttctgctg

SEQ ID NO: 1220 - protein, 15 aa, Arachis hypogaea
SYGRDPYSPSQDPYS

SEQ ID NO: 1221 - DNA, 45 bp, Arachis hypogaea
  source 1..45: mol_type = other DNA
agctatggccgcgatccgtatagcccgagccaggatccgtatagc

SEQ ID NO: 1222 - protein, 15 aa, Arachis hypogaea
QDRQMVQQFKRELMN

SEQ ID NO: 1223 - DNA, 45 bp, Arachis hypogaea
  source 1..45: mol_type = other DNA
caggatcgccagatggtgcagcagtttaaacgcgaactgatgaac

SEQ ID NO: 1224 - protein, 15 aa, Arachis hypogaea
SAMRRERGRQGDSSS

SEQ ID NO: 1225 - DNA, 45 bp, Arachis hypogaea
  source 1..45: mol_type = other DNA
agcgcgatgcgccgcgaacgcggccgccagggcgatagcagcagc

SEQ ID NO: 1226 - protein, 15 aa, Arachis hypogaea
QYDSYDIRSTRSSDQ

SEQ ID NO: 1227 - DNA, 45 bp, Arachis hypogaea
  source 1..45: mol_type = other DNA
cagtatgatagctatgatattcgcagcacccgcagcagcgatcag

SEQ ID NO: 1228 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 1229 - protein, 118 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDV

SEQ ID NO: 1230 - DNA, 354 bp, Mus musculus
  source 1..354: mol_type = other DNA
atgatggatcaggcgcgcagcgcgtttagcaacctgtttggcggcgaaccgctgagctatacccgctttagcctggcgcgccaggtggatggcgataacagccatgtggaaatgaaactggcggcggatgaagaagaaaacgcggataacaacatgaaagcgagcgtgcgcaaaccgaaacgctttaacggccgcctgtgctttgcggcgattgcgctggtgattttttttctgattggctttatgagcggctatctgggctattgcaaacgcgtggaacagaaagaagaatgcgtgaaactggcggaaaccgaagaaaccgataaaagcgaaaccatggaaaccgaagatgtg

SEQ ID NO: 1231 - DNA, 720 bp, synthetic construct
  misc_feature 1..720: note = DNA sequence encoding mouse Ara h2 epitopes, separated by cleavable linkers
  source 1..720: mol_type = other DNA
atgatgatggatcaggcgcgcagcgcgtttagcaacctgtttggcggcgaaccgctgagctatacccgctttagcctggcgcgccaggtggatggcgataacagccatgtggaaatgaaactggcggcggatgaagaagaaaacgcggataacaacatgaaagcgagcgtgcgcaaaccgaaacgctttaacggccgcctgtgctttgcggcgattgcgctggtgattttttttctgattggctttatgagcggctatctgggctattgcaaacgcgtggaacagaaagaagaatgcgtgaaactggcggaaaccgaagaaaccgataaaagcgaaaccatggaaaccgaagatgtgggcggcggcggcagccgcgtgcgccgcggcggcggcggcagcctggcgctgtttctgctggcggcgcatgcgagcgcgcgccagcagggcggcggcggcagccgcgtgcgccgcggcggcggcggcagcctgcgcaacctgccgcagcagtgcggcctgcgcgcgccgcagcgcggcggcggcggcagccgcgtgcgccgcggcggcggcggcagcatggcgaaactgaccattctggtggcgctggcgctgtttctgctgggcggcggcggcagccgcgtgcgccgcggcggcggcggcagcagctatggccgcgatccgtatagcccgagccaggatccgtatagccgcgtgcgccgctaa

SEQ ID NO: 1232 - DNA, 982 bp, synthetic construct
  misc_feature 1..982: note = DNA sequence encoding mouse Ara h2 epitopes, separated by cleavable linkers
  source 1..982: mol_type = other DNA
atgatgatggatcaggcgcgcagcgcgtttagcaacctggtttggcggcgaaccgctgagctatacccgctttagcctggcgcgccaggtggatggcgataacagccatgtggaaatgaaactggcggcggatgaagaagaaaacgcggataacaacatgaaagcgagcgtgcgcaaaccgaaacgctttaacggccgcctgtgctttgcggcgattgcgctggtgattttttttctgattggctttatgagcggctatctgggctattgcaaacgcgtggaacagaaagaagaatgcgtgaaactggcggaaaccgaagaaaccgataaaagcgaaaccatggaaaccgaagatgtgggcggcggcggcagccgcgtgcgccgcggcggcggcggcagcctggcgctgtttctgctggcggcgcatgcgagcgcgcgccagcagggcggcggcggcagccgcgtgcgccacggcggcggcggcagcctgcgcaacctgccgcagcagtgcggcctgcgcgcgccgcagcgcggcggcggcggcagccgcgtgcgccgcggcggcggcggcagcatggcgaaactgaccattctggtggcgctggcgctgtttctgctgggcggcggcggcagccgcgtgcgccgcggcagcggcggcagcagctatggccgcgatccgtatagcccgagccaggatccgtatagcggcggcggcggcagccgcgtgcgccgcggcggcggcggcagccaggatcgccagatggtgcagcagtttaaacgcgaactgatgaacggcggcggcggcagccgcgtgcgccgcggcggcggcggcagcagcgcgatgcgccgcgaacgcggccgccagggcgatagcagcagcggcggcggcggcagccgcgtgcgccgcggcggcggcggcagccagtatgatagctatgatattcgcagcacccgcagcacgaatcagcgcgtgcgccgctaa

SEQ ID NO: 1233 - protein, 15 aa, Homo sapiens
PSLRTLEDNEERMSR

SEQ ID NO: 1234 - protein, 15 aa, Synthetic construct
SYQPLGDKVNFFRMV

SEQ ID NO: 1235 - DNA, 720 bp, synthetic construct
  misc_feature 1..720: note = Codon optimized DNA sequence encoding mouse Ara h2 epitopes, separated by cleavable linkers
  source 1..720: mol_type = other DNA
atgatgatggaccaggctcgttcagcctttagtaacctttttggaggtgagccgctgagttacacccgcttctccttggcacgacaagtcgacggagataacagccatgtggagatgaaactcgctgcagatgaagaggagaatgctgacaacaacatgaaggcctctgtgaggaaaccaaaaaggttcaatggccggctctgctttgctgccatagccctggtcatcttcttcctgattggctttatgagcggctacctggggtactgcaagagagtagagcagaaggaagaatgtgtgaaactagcagagacagaggaaactgacaagagtgaaaccatggagactgaagatgtgggaggaggcggctcccgtgtaaggaggggcgggggcggatccctggccctctttttactagcggctcacgccagcgcacgccagcaagggggtggcggttcaagagttcgcagaggtggaggggggtctcttcggaatttgccacagcagtgtggcctgagggccccccagagggggggaggtggctctagagtcagacgtggtggtggggggtccatggccaagctgacaatcctggtggctcttgcattgttcctcctgggcggagggggcagccgcgtgcggcgaggcggtgggggatcatcctatggaagagatccctattctcctagccaggacccttattcgcgagttcggcggtga

SEQ ID NO: 1236 - DNA, 981 bp, synthetic construct
  misc_feature 1..981: note = Codon optimized DNA sequence encoding mouse Ara h2 epitopes, separated by cleavable linkers
  source 1..981: mol_type = other DNA
atgatgatggaccaggcccgctcggccttcagcaacctgtttgggggagagccattgagttacactaggtttagcttggcaaggcaggtggatggggacaattcccatgtggagatgaaactcgccgcagatgaagaggaaaatgctgacaacaacatgaaggcctctgtgaggaaacccaaaaggttcaatggcaggctgtgctttgctgcgattgctcttgtcattttctttctcatagggttcatgagcggctacctgggctactgcaaaagagtcgagcagaaggaagaatgtgtgaagctggccgagacagaagaaacggacaagagtgagaccatggagactgaagatgtgggcggcggaggctctcgggtcagaaggggagggggtggatcccttgcactatttttattagcagcccacgcgtctgcccggcaacaagggggaggcggctctcgagtgagacgagggggcggtggcagcctgaggaaccttcctcagcagtgtggcctgcgcgcaccacagcgcggtggcggaggatcgcgtgttcggagaggaggaggaggcagcatggccaagttgaccatcctggtggctctagctctcttcctgctcggcggagggggttcacgggtccgtcgtggcgggggtgggtctagctatggtagagatccttatagtccgtctcaggacccctacagtgggggcggaggttcacgtgtacgccgaggaggaggggggagccaagacagacagatggtgcagcagttcaagcgggagctgatgaatggcggtggcggcagccgcgttagacgagggggcgggggatcatcagctatgagaagagagcggggtcgacaaggagattccagcagtgggggagggggctcacgggtacgccgcggaggtggaggttcccagtacgactcctatgacatcagatcaacaaggtcctccgatcagcgggttagaaggtga

SEQ ID NO: 1237 - RNA, 362 nt, Synthetic construct
  source 1..362: mol_type = mRNA
atggatgaccagcgagatctcattagcaaccacgagcagctgcccatactgggcaatcgccctagagagccagaaagatgctccaggggtgctctttacaccggagtcagtgttctggtggccttgttactagcaggacaggctactacagcctacttcctctaccagcagcaagggcggctggacaagcttaccatcacaagccagaacctgcagttggaaagtctacgcatgaagctcaaacgggaagattcctatgggcgtgatccttacagtccgtcacaagatccttattccccatctgaggacagctatggcagggacccatattctccttctcaagacccctactcacccagcta

SEQ ID NO: 1238 - RNA, 765 nt, Synthetic construct
  source 1..765: mol_type = other RNA
atggacgaccagcgcgacctgatctccaaccacgagcagctgcccatcctgggcaaccgcccccgcgagcccgagcgctgctcccgcggcgccctgtacaccggcgtgtccgtgctggtggccctgctgctggccggccaggccaccaccgcctacttcctgtaccagcagcagggccgcctggacaagctgaccatcacctcccagaacctgcagctggagtccctgcgcatgaagctgaagcgctgcggctcccacctggtggagcgcctgtacctggtgtgcggcgagcgcggcttcttctccctgaagaagggcgccgccgccctgggcatcggcaccgactccgtgatcctgatctgcggctcccacctggtggagcgcctgtacctggtgtgcggcgaggagggcttcttctgcggctcccacctggtggagcgcctgtacctggtgtgcggcggcgagggcttcttcaagcgcgccgtgccccccctgtgggtgcgcatggagaagcgcaagcgctacgtgcgccccctgtgggtgcgcatggagaagcgcaagcgctggtcccgcatggaccagctggccaaggagctgaccgccgagaagcgcaagcgcggctccggcgagggccgcggctccctgctgacctgcggcgacgtggaggagaaccccggccccggcgacctgcagaccctggccctgtggtcccgcatggaccagctggagcgcctgtacctggtgtgcggcgagcgcggcttctaa

(19) United States
(12) Patent Application Publication
(10) Pub. No.: US 2025/0339514 A1
(43) Pub. Date: Nov. 6, 2025

(54) SINGLE-AND MULTI-EPITOPE PEPTIDE AND MRNA VACCINES TO GENERATE TOLEROGENIC EFFECTS FOR ALLERGIC AND AUTOIMMUNE DISEASE BY TARGETING LIVER SINUSOIDAL ENDOTHELIAL CELLS

(71) Applicant: The Regents of the University of California, Oakland, CA (US)

(72) Inventors: Andre E. Nel, Sherman Oaks, CA (US); Tian Xia, Los Angeles, CA (US); Qi Liu, Los Angeles, CA (US); Xiang Wang, Los Angeles, CA (US)

(73) Assignee: The Regents of the University of California, Oakland, CA (US)

(21) Appl. No.: 18/579,853

(22) PCT Filed: Jul. 18, 2022
(86) PCT No.: PCT/US2022/037506

§ 371 Date: Jan. 16, 2024

(51) Int. Cl. A61K 39/35; A61K 39/00; A61K 39/385; A61P 37/08; C07K 14/415; C07K 14/77

(52) U.S. Cl. CPC A61K 39/35; A61K 39/001; A61K 39/385; A61P 37/08; C07K 14/415; C07K 14/77; A61K 2039/55555; A61K 2039/577

(57) ABSTRACT

In various embodiments tolerogenic nanoparticles are provided that induce immune tolerance to one or more desired antigen(s) and/or that reduce an immune response to those antigen(s). In certain embodiments the tolerogenic nanoparticle comprises a nanoparticle comprising a biocompatible polymer, a cationic lipid, or a combination thereof; an antigen disposed within or attached to said biocompatible polymer or a nucleic acid encoding said antigen, where said antigen comprises an antigen to which immune tolerance is to be induced by administration of said tolerogenic nanoparticle to a mammal; and a targeting moiety that binds to a scavenger receptor in the liver.

### CROSS-REFERENCE TO RELATED APPLICATIONS

This application is a National Phase Application of PCT International Application No. PCT/US2022/037506, International Filing Date Jul. 18, 2022, which claims benefit of and priority to U.S. Ser. No. 63/223,936, filed on Jul. 20, 2021, which are incorporated herein by reference in their entirety for all purposes.

### STATEMENT OF GOVERNMENTAL SUPPORT

This invention was made with government support under Grant Numbers ES022698 and ES027237, awarded by the National Institutes of Health. The government has certain rights in the invention.

### INCORPORATION BY REFERENCE OF SEQUENCE LISTING

The instant application contains a Sequence Listing conforming the rules of WIPO Standard ST.26 which has been submitted electronically in XML format and is hereby incorporated by reference in its entirety. The XML copy, created on Jan. 24, 2025, is named “P-635468-US_SQL_28JAN25.xml” and is 1,125,404 bytes in size.

### BACKGROUND

There is an unmet need for developing new treatment approaches for autoimmune and allergic disorders that goes beyond current therapeutic efforts of utilizing anti-inflammatory, immunosuppressive, targeted monoclonal antibody, or immunomodulatory approaches. Although most of these therapies provide symptomatic relief and a temporary abatement of disease activity, they do not provide long-term suppression of chronic disease activity or the prospect of a cure.

However, there is growing awareness of the power of regulatory T-cell (Treg) biology to provide antigen-specific immune tolerance for autoimmune diseases (e.g., rheumatoid arthritis, lupus, type I diabetes) and allergic disorders (e.g., food allergy (e.g., peanut, wheat, milk, egg, etc.), anaphylaxis, asthma) (Finkelman (2010) Curr. Opin. Immunol. 22 (6): 783-788; Wang & Sampson (2007) Clin. Exp. Allergy, 37 (5): 651-660). One approach for inducing antigen-specific tolerance is to use biodegradable nanoparticles to initiate and sustain immunomodulatory responses, based on the ability of these carriers to encapsulate disease-related antigens that are delivered to antigen-presenting cells (APC) (Sicherer & Sampson (2018) J. Allergy Clin. Immunol. 141 (1): 41-58; Branum & Lukacs (2009) Pediatrics, 124 (6): 1549-1555; Bock et al. (2001) J. Allergy Clin. Immunol. 107 (1): 191-193; Boyce et al. (2010) J. Allergy Clin. Immunol. 126 (6): 1105-1118; Varshney et al. (2011) J. Allergy Clin. Immunol. 127 (3): 654-660). The tolerogenic properties of the liver are well-known for this organ's role in preventing immune responses to exogenous food antigens coming from the gastrointestinal tract and portal venous system, as well as promoting the persistence of tumor metastases to this organ (Chin et al. (2013) J. Allergy Clin. Immunol. 132 (2): 476-478.e2). Moreover, the liver also enjoys immune privilege during organ transplantation, requiring less immunosuppressive therapy than kidney or heart transplants (Nurmatov et al. (2012) Cochrane Database Syst. Rev. (9)). It has also been demonstrated that concurrent transplant of a kidney or a heart with a liver is less prone to undergo immunological rejection compared to an isolated organ transplant (Sheikh & Burks (2013) Expert Rev. Clin. Immunol. 9 (6): 551-560.; Dougherty et al. (2021) Ann. Pharmacother. 55 (3): 344-353). The hepatic expression and ability of liver APCs to present myelin basic protein (MBP) to the immune system has likewise been demonstrated to control experimental allergic encephalomyelitis (an autoimmune disorder that simulates multiple sclerosis) in mice (Vickery et al. (2018) N. Engl. J. Med. 379 (21): 1991-2001).

The immunosuppressive effects of the liver can, in part, be ascribed to its unique system of APCs, including natural tolerogenic APCs such as Kupffer cells (KC), dendritic cells (DC), and liver sinusoidal endothelial cells (LSECs) (Santos et al. (2020) J. Allergy Clin. Immunol. 145 (1): 440-443.e5; Mullard (2020) Nat Rev Drug Discov. 19 (3): 156). These tolerogenic APCs constitute an integral component of the liver's reticuloendothelial system, which has the key function of clearing foreign materials, degradation products, and toxins from sinusoidal blood by phagocytic uptake as well as endocytic processing (Mullard (2020) supra.). Moreover, whereas professional phagocytes (KC and DC) preferentially eliminate circulating microscale particulate materials through phagocytosis, LSECs are more proficient in eliminating soluble macromolecules and particulates in the 200 nm size range by clathrin-mediated endocytosis (Patrawala et al. (2020) Curr. Allergy Asthma Rep. 20 (5): 14; Wasserman et al. (2021) J. Allergy Clin. Immunol: In Practice, 9 (5): 1826-1838.e8). From an immunoregulatory perspective, LSECs play a key role in inducing immune suppression of CD8⁺ and CD4⁺ populations through the generation ofantigen-specific Tregs, TGF-β production, and upregulation of the ligand (PD-L1) for the programmed cell death protein 1 (PD-1) receptor (Akdis (2012) Nat. Med. 18 (5): 736-749; Johnson-Weaver et al. (2018) Front. Immunol. 9:2156; Liu et al. (2019) ACS Nano, 13 (4): 4778-4794). It is also of further interest that LSECs obtained from Foxp3gfp/KI transgenic mice were shown to be more capable of generating antigen-specific CD4⁺/Foxp3⁺ regulatory T-cells compared to KC or liver DC from the same animals (Johnson-Weaver et al. supra; Liu et al. (2021) ACS Nano, 15 (1): 1608-1626). Thus, the ability of LSECs to control the function of antigen-specific Tregs should be considered for the treatment of autoimmune and allergic disease manifestations.

The use of nanoparticles to induce immune tolerance is an active area of investigation and includes approaches such as decorating particle surfaces with peptide/major histocompatibility (MHC) complexes, serving as a surrogate antigen presentation platform for immune tolerization in the absence of costimulation (Tiegs & Lohse (2010) J. Autoimmun. 34 (1): 1-6; Vickery et al. (2011) J. Allergy & Clin. Immunol. 127 (3): 576-586; Knolle & Wohlleber (2016) Cell. Mol. Immunol. 13 (3): 347-353; Crispe, (2011) J. Hepatol. 54 (2): 357-365; Horst et al. (2016) Cell. Mol. Immunol. 13 (3): 277-292). Other approaches include the incorporation of food allergens or autoimmune proteins/peptides (e.g., type II collagen) in orally administered nanoparticles (Carambia et al. (2014) J. Hepatol. 61 (3): 594-9; Carambia et al. (2015) J. Hepatol. 62 (6): 1349-1356; Hemmings et al. (2020) J. Allergy Clin. Immunol. 146 (3): 621-630.e5), harnessing apoptotic cell death (e.g., apoptotic cell-peptide conjugates or liposomes containing phosphatidylserine) (see, e.g., Li et al. (2000) J. Allergy Clin. Immunol. 106 (1, Part 1): 150-158; Srivastava et al. (2016) J. Allergy Clin. Immunol. 138 (2): 536-543.e4; Prickett et al. (2011) J. Allergy Clin. Immunol. 127 (3): 608-615.e5; Orgel & Kulis (2018) A Mouse Model of Peanut Allergy Induced by Sensitization Through the Gastrointestinal Tract. In Type 2 Immunity: Methods and Protocols, Reinhardt, R. L., Ed. Springer New York: New York, NY, pp 39-47; Kishimoto & Maldonado (2018) Front. Immunol. 9:230), targeting B-cell-specific tolerance via the CD22 receptor (see, e.g., Nguyen-Lefebvre & Horuzsko (2015) J. Enzymol. Metab. 1 (1): 101; Pandey et al. (2020) Front. Physiol. 11:873-873), or encapsulating pharmacological agents (e.g., rapamycin) that induce tolerogenic states in APC by impacting antigen presentation, maturation, and/or the expression of costimulatory molecules (see, e.g., Sørensen et al. (2012) Am. J. Physiol. Regul. Integr. Comp. Physiol. 303 (12): R1217-R1230; Park et al. (2016) Nanomed. Nanotechnol. Biol. Med. 12 (5): 1365-1374; Prickett et al. (2015) Clin. Exp. Allergy, 45 (6): 1015-1026).

### SUMMARY

Although various nano-enabled immunotherapy approaches are yielding promising results, our preferred approach is to use the natural tolerogenic effects of the liver, which can be exploited by a versatile nanoparticle platform constructed from, inter alia, a FDA-approved biodegradable polymer, e.g., poly(lactic-co-glycolic acid) (PLGA) and/or lipid (e.g., a cationic lipid). One illustrative approach is to target mannose and/or scavenging receptors (SR) that are involved in endocytosis of circulating antigens, extracellular macromolecules, protein degradation products, and lipoproteins by LSECs. Whereas the stabilin-1 and stabilin-2 SRs are exclusively expressed on LSECs, the mannose receptor also appears in lesser quantities on the KC surface. These receptors can be targeted by placing, for example, an apolipoprotein B (ApoB) peptide sequence or mannan, respectively, on the nanoparticle surface.

As described in the Examples with respect to an illustrative, but non-limiting embodiment, we demonstrate the design and synthesis of tolerogenic nanoparticles for delivering one or more peptide epitopes or for delivering a nucleic acid encoding a sequence for one or more peptide epitopes to LSECs resulting in the generation of immune tolerance to the epitope(s) provided in the nanoparticles, or to the epitopes encoded by nucleic acid(s) provided in the nanoparticles.

Accordingly, various embodiments provided herein may include, but need not be limited to, one or more of the following:

Embodiment 1: A tolerogenic nanoparticle comprising:
a nanoparticle comprising a biocompatible polymer;
a peptide epitope comprising an antigen or antigen fragment to which immune tolerance is to be induced by administration of said tolerogenic nanoparticle to a mammal, where said peptide epitope comprises an epitope selected from the group consisting an epitope from a peanut allergen, an epitope from a house dust mite allergen, an adalimumab T cell epitope, a pancreatic beta cell autoantigen epitope, a histone epitope, and a viral vector T cell epitope, where said epitope is encapsulated within or attached to said biocompatible polymer; and
a targeting moiety attached to the surface of said nanoparticle where said targeting moiety binds to a liver cell.

Embodiment 2: A tolerogenic nanoparticle comprising:
a nanoparticle comprising one or more lipid(s) and/or one or more biocompatible polymer(s);
a nucleic acid encoding a peptide epitope comprising an antigen or antigen fragment to which immune tolerance is to be induced by administration of said tolerogenic nanoparticle to a mammal, where said peptide epitope comprises an epitope selected from the group consisting an epitope from a peanut allergen, an epitope from a house dust mite allergen, an adalimumab T cell epitope, a pancreatic beta cell autoantigen epitope, a histone epitope, and a viral vector T cell epitope, where said nucleic acid is encapsulated within or attached to the surface of said nanoparticle; and
wherein said nanoparticle comprises or consists of lipids that accumulate in the liver and/or said nanoparticle comprises a targeting moiety attached to the surface of said nanoparticle where said targeting moiety binds to a liver cell.

Embodiment 3: The tolerogenic nanoparticle of embodiment 2, wherein said nanoparticle comprises or consists of one or more biocompatible polymers.

Embodiment 4: The tolerogenic nanoparticle of embodiment 2, wherein said nanoparticle comprises or consists of one or more lipids.

Embodiment 5: The tolerogenic nanoparticle according to any one of embodiments 1-4, wherein said wherein said tolerogenic nanoparticle comprises one or more biocompatible polymers selected from the group consisting of poly(lactic-co-glycolic acid) (PLGA), Poly(glycolic acid) (PGA), Poly(lactic acid) (PLA), Poly(caprolactone) (PCL), Poly(butylene succinate), Poly(trimethylene carbonate), Poly(p-dioxanone), Poly(butylene terephthalate), Poly(ester amide) (HYBRANE®), polyurethane, Poly[(carboxyphenoxy) propane-sebacic acid], Poly[bis(hydroxyethyl) terephthalate-ethyl orthophosphorylate/terephthaloyl chloride], Poly(β-hydroxyalkanoate), Poly(hydroxybutyrate), and Poly(hydroxybutyrate-co-hydroxyvalerate).

Embodiment 6: The tolerogenic nanoparticle of embodiment 5, wherein said biocompatible polymer comprises poly(lactic-co-glycolic acid) (PLGA).

Embodiment 7: The tolerogenic nanoparticle of embodiment 6, wherein said PLGA comprises a lactide/glycolide molar ratio of about 50:50.

Embodiment 8: The tolerogenic nanoparticle according to any one of embodiments 6-7, wherein said PLGA includes a content ranging from about 8% up to about 20% of ˜5 kDa PEG.

Embodiment 9: The tolerogenic nanoparticle according to any one of embodiments 1-8, wherein said tolerogenic nanoparticle comprises or consists of one or more cationic polymers.

Embodiment 10: The tolerogenic nanoparticle of embodiment 9, wherein said nanoparticle comprises or consists of one or more cationic polymers selected from the group consisting of poly-L-lysine (PLL), poly-ethylenimine (PEI, poly[(2-dimethylamino) ethyl methacrylate] (pDMAEMA), polyamidoamine (PAMAM) dendrimers, poly(β-amino ester) (PBAE) polymer, poly(amino-co-ester) (PACE)-based polymers.

Embodiment 11: The tolerogenic nanoparticle according to any one of embodiments 1-10, wherein said tolerogenic nanoparticle comprises one or more cationic lipids.

Embodiment 12: The tolerogenic nanoparticle of embodiment 11, wherein said nanoparticle comprises one or more cationic lipids selected from the group consisting of dilinoleylmethyl-4 dimethyl aminobutyrate (DLin-MC3-DMA), 1,2-dioleoyl-3-dimethylaminopropane (DODAP), didodecyl-dimethylammonium bromide (DDAB), 1,2-dioleoyloxy-3-trimethylammonium propane chloride (DOTAP), (N-[1-(2,3-dioleyloxy)propyl]-N,N,N-trimethylammonium chloride (DOTMA), 1,2-dilinoleyloxy-3-dimethylaminopropane (DLinDMA), 2,2-dilinoleyl-4-dimethylaminomethyl-[1,3]-dioxolane (DLin-K-DMA), 2,2-dilinoleyl-4-(2-dimethylaminoethyl)-[1,3]-dioxolane (DLin-KC2-DMA), C12-200 (Lipid 5), 3-(dimethylamino)propyl (12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl) carbonyloxy]nonadecanoate (L101), dicetylphosphate-tetraethylenepentaamine-based polycation lipid, YSK05, YSK12-C4, YSK13-C4, and YSK15-C4.

Embodiment 13: The tolerogenic nanoparticle according to any one of embodiments 2-12, wherein said tolerogenic nanoparticle comprises a lipidic nanoparticle (LNP).

Embodiment 14: The tolerogenic nanoparticle of embodiment 13, wherein said lipidic nanoparticle comprises a helper lipid.

Embodiment 15: The tolerogenic nanoparticle of embodiment 14, wherein said helper lipid comprises a lipid selected from the group consisting of 1,2-distearoyl-sn-glycero-3-phosphocholine (DSPC), 1,2-dioleoyl-sn-glycero-3-phosphoethanolamine (DOPE), and 1-Palmitoyl-2-oleoyl-sn-glycero-3-phosphoethanolamine (POPE).

Embodiment 16: The tolerogenic nanoparticle according to any one of embodiments 11-15, wherein said lipidic nanoparticle comprises cholesterol hemisuccinate (CHEMS).

Embodiment 17: The tolerogenic nanoparticle according to any one of embodiments 11-15, wherein said lipidic nanoparticle comprises cholesterol.

Embodiment 18: The tolerogenic nanoparticle according to any one of embodiments 11-17, wherein said lipidic nanoparticle comprises a PEG-lipid.

Embodiment 19: The tolerogenic nanoparticle according to any one of embodiments 11-18, wherein said lipidic nanoparticle comprises an ionizable (e.g., a cationic) lipid, a helper lipid, a PEG-lipid, and CHEMS.

Embodiment 20: The tolerogenic nanoparticle according to any one of embodiments 11-18, wherein said lipidic nanoparticle comprises an ionizable (e.g., a cationic) lipid, a helper lipid, a PEG-lipid, and cholesterol.

Embodiment 21: The tolerogenic nanoparticle of embodiment 20, wherein said lipidic nanoparticle comprises DLin-MC3-DMA, DSPC, cholesterol, and a PEG lipid.

Embodiment 22: The tolerogenic nanoparticle of embodiment 21, wherein said lipidic nanoparticle comprises DLin-MC3-DMA, DSPC, cholesterol, and a DMG-PEG2000.

Embodiment 23: The tolerogenic nanoparticle of embodiment 22, wherein the molar ratio of DLin-MC3-DMA: DSPC: cholesterol: DMB-PEG2000 is 50:10: 38.5:1.5.

Embodiment 24: The tolerogenic nanoparticle of embodiment 20, wherein said lipidic nanoparticle comprises cholesterol, distearoylphosphatidylcholine (DSPC), PEG-Lipid, and DODAP.

Embodiment 25: The tolerogenic nanoparticle of embodiment 20, wherein said lipidic nanoparticle comprises Lin-DMA, DSPC, cholesterol, PEG2000-C-DMG.

Embodiment 26: The tolerogenic nanoparticle of embodiment 20, wherein said lipidic nanoparticle comprises DLin-MC3-DMA, DSPC, cholesterol, and PEG2000-C-DMG).

Embodiment 27: The tolerogenic nanoparticle according to any one of embodiments 25-26, where the molar ratio of ionizable cationic lipid: DSPC: Cholesterol: PEG2000-C-DMG is 50:10: 39:5: 1.5 mol %.

Embodiment 28: The tolerogenic nanoparticle of embodiment 20, wherein said lipidic nanoparticle comprises lipidoid 98N12-5 (1) 4HCl, cholesterol, and mPEG2000-DMG.

Embodiment 29: The tolerogenic nanoparticle of embodiment 28, wherein the molar ratio of lipidoid 98N12-5 (1) 4HCl: cholesterol: mPEG2000-DMG is 42:48:10.

Embodiment 30: The tolerogenic nanoparticle of embodiment 20, wherein said lipidic nanoparticle comprises DLin-MC3-DMA, cholesterol, and mPEG2000-DMG.

Embodiment 31: The tolerogenic nanoparticle of embodiment 30, wherein the molar ratio of DLin-MC3-DMA: cholesterol: mPEG2000-DMG is 42:48: 10.

Embodiment 32: The tolerogenic nanoparticle of embodiment 20, wherein said lipidic nanoparticle comprises an ionizable lipid, a helper lipid, cholesterol, and PEG-DMG, where said ionizable lipid is selected from the group consisting of YSK05, YSK12-C4, YSK13-C4, and YSK15-C4.

Embodiment 33: The tolerogenic nanoparticle of embodiment 32, wherein said ionizable lipid comprises YSK05.

Embodiment 34: The tolerogenic nanoparticle according to any one of embodiments 32-33, wherein said lipid nanoparticle comprises a molar ratio of ionizable lipid: helper lipid: cholesterol: PEG-DMG of 50:10: 40:3.

Embodiment 35: The tolerogenic nanoparticle according to any one of embodiments 32-33, wherein said lipid nanoparticle comprises a molar ratio of ionizable lipid: cholesterol: PEG-DMG of 70:30: 3.

Embodiment 36: The tolerogenic nanoparticle according to any one of embodiments 1-8, wherein said tolerogenic nanoparticle comprises the cationic lipid, dioleoyl-3-trimethylammonium propane (DOTAP), and the cationic polymer protamine.

Embodiment 37: The tolerogenic nanoparticle according to any one of embodiments 1-36, wherein said nanoparticle ranges in size from about 50 nm, or from about 100 nm, or from about 200 nm up to about 450 nm, or up to about 400 nm, or up to about 350 nm, or up to about 300 nm.

Embodiment 38: The tolerogenic nanoparticle of embodiment 37, wherein said nanoparticle ranges in size from about 200 nm up to about 300 nm, or from about 100 nm up to about 170 nm, or from 100 nm up to about 130 nm.

Embodiment 39: The tolerogenic nanoparticle according to any one of embodiments 1-38, wherein said nanoparticle comprises said targeting moiety that binds to an APC displaying a scavenger receptor in the liver.

Embodiment 40: The tolerogenic nanoparticle of embodiment 39, wherein said targeting moiety binds to a liver sinusoidal endothelial cell (LSEC).

Embodiment 41: The tolerogenic nanoparticle of embodiment 39, wherein said targeting moiety binds to or more scavenger receptors selected from the group consisting of Stabilin 1, Stabilin 2, and a mannose receptor.

Embodiment 42: The tolerogenic nanoparticle of embodiment 41, wherein said targeting moiety comprises mannose.

Embodiment 43: The tolerogenic nanoparticle of embodiment 41, wherein said targeting moiety comprises a fragment of apolipoprotein B protein effective to bind to Stabilin 1 and/or Stabilin 2.

Embodiment 44: The tolerogenic nanoparticle of embodiment 43, wherein said targeting moiety fragment ranges in length from about 5, or from about 8, or from about 10 up to about 50, or up to about 40, or up to about 30, or up to about 20 amino acids.

Embodiment 45: The tolerogenic nanoparticle of embodiment 44, wherein said targeting moiety comprises a fragment of the apoB protein comprising the amino acid sequence RKRGLK (SEQ ID NO:18).

Embodiment 46: The tolerogenic nanoparticle of embodiment 45, wherein said targeting moiety comprises a fragment of the apoB protein comprising the amino acid sequence RLYRKRGLK (SEQ ID NO: 19).

Embodiment 47: The tolerogenic nanoparticle of embodiment 45, wherein said targeting moiety comprise or consists of the amino acid sequence CGGKLGRKYRYLR (SEQ ID NO:4).

Embodiment 48: The tolerogenic nanoparticle according to any one of embodiments 1-47, wherein said targeting moiety is physically adsorbed to said nanoparticle.

Embodiment 49: The tolerogenic nanoparticle according to any one of embodiments 1-47, wherein said targeting moiety is covalently bound to said nanoparticle directly or through a linker.

Embodiment 50: The tolerogenic nanoparticle of embodiment 49, wherein targeting moiety is covalently bound to said nanoparticle through a linker.

Embodiment 51: The tolerogenic nanoparticle of embodiment 50, wherein said linker comprises a maleimide linker.

Embodiment 52: The tolerogenic nanoparticle of embodiment 51, wherein said linker comprises N-(2-aminoethyl) maleimide (NAEM).

Embodiment 53: The tolerogenic nanoparticle of embodiment 49, wherein said targeting moiety is bound to a lipid comprising said nanoparticle.

Embodiment 54: The tolerogenic nanopartiocle, wherein said targeting moiety is bound to to a pegylated lipid, a cationic lipid, and/or to cholesterol and/or CHEMS.

Embodiment 55: The tolerogenic nanoparticle of embodiment 54, wherein said targeting moiety comprises mannose attached to a pegylated lipid.

Embodiment 56: The tolerogenic nanoparticle of embodiment 55, wherein said targeting moiety comprises DSPE-PEG2000-Mannose.

Embodiment 57: The tolerogenic nanoparticle according to embodiment 56, wherein said nanparticle is a lipidic nanoparticle compriseing Dlin-MC3-DMA, DSPC, Cholesterol, and DSPE-PEG-Man.

Embodiment 58: The tolerogenic nanoparticle according to embodiment 57, wherein the molar ratio of Dlin-MC3-DMA: DSPC: Cholesterol: DSPE-PEG-Man is 50:10: 38.5:1.5.

Embodiment 59: The tolerogenic nanoparticle according to any one of embodiments 1-58, wherein said nanoparticle comprises a second targeting moiety that binds to a mannose receptor.

Embodiment 60: The tolerogenic nanoparticle of embodiment 59, wherein said second targeting moiety comprises mannan or mannose.

Embodiment 61: The tolerogenic nanoparticle of embodiment 60, wherein said second targeting moiety comprises a mannan having a MW ranging from about 35 to about 60 kDa.

Embodiment 62: The tolerogenic nanoparticle according to any one of embodiments 59-61, wherein said second binding moiety is adsorbed to said nanoparticle.

Embodiment 63: The tolerogenic nanoparticle according to any one of embodiments 59-61, wherein said second binding moiety is covalently bound to said nanoparticle directly or through a linker.

Embodiment 64: The tolerogenic nanoparticle of embodiment 59, wherein said second binding moiety is coupled to said nanoparticle through a hydroxyl terminus of said binding moiety.

Embodiment 65: The tolerogenic nanoparticle of embodiment 64, wherein said hydroxyl terminus is bound to a COOH terminal group on said nanoparticle.

Embodiment 66: The tolerogenic nanoparticle according to any one of embodiments 59-65, wherein said nanoparticle comprises a third targeting moiety that binds to a hepatocyte.

Embodiment 67: The tolerogenic nanoparticle of embodiment 66, wherein said third targeting moiety comprises a moiety selected from the group consisting of Asialoorosomucoid, Galactoside, a Galactosamine, Asialofetuin, Sterylglucoside, Lactose/lactobionic acid, PVLA (poly-(N-p-vinylbenzyl-O-beta-D-galactopyranosyl-[1-4]-D-gluconamide), Linoleic acid, Glycyrrhizin, and acetyl-CKNEKKNKIERNNKLKQPP-amide (SEQ ID NO:20).

Embodiment 68: The tolerogenic nanoparticle of embodiment 67, wherein said third targeting moiety comprises N-acetylgalactosamine (GalNAC).

Embodiment 69: The tolerogenic nanoparticle according to any one of embodiments 66-68, wherein said third binding moiety is adsorbed to said nanoparticle.

Embodiment 70: The tolerogenic nanoparticle according to any one of embodiments 66-68, wherein said third binding moiety is covalently bound to said nanoparticle directly or through a linker.

Embodiment 71: The tolerogenic nanoparticle according to any one of embodiments 1-70, wherein said peptide epitope comprises a peptide epitope that shows affinity binding to HLA class II molecules.

Embodiment 72: The tolerogenic nanoparticle according to any one of embodiments 1-70, wherein said peptide epitope comprises one or more peanut peptide epitopes selected from the epitopes shown in Table 2, and/or Table 3, and/or Table 4, and/or Table 5, and/or Table 6, and/or Table 7, and/or Table 8, and/or Table 9, and/or Table 10.

Embodiment 73: The tolerogenic nanoparticle according to any one of embodiments 2-70, wherein said nucleic acid encodes a peptide epitope comprising one or more peanut peptide epitopes selected from the epitopes shown in Table 2, and/or Table 3, and/or Table 4, and/or Table 5, and/or Table 6, and/or Table 7, and/or Table 8, and/or Table 9, and/or Table 10.

Embodiment 74: The tolerogenic nanoparticle according to any one of embodiments 72-73, wherein said peptide epitope comprises a single peanut allergen epitope selected from the epitopes shown in Table 2, and/or Table 3, and/or Table 4, and/or Table 5, and/or Table 6, and/or Table 7, and/or Table 8, and/or Table 9, and/or Table 10; or said tolerogenic nanoparticle comprises a nucleic acid that encodes a single peptide epitope selected from the epitopes shown in Table 2, and/or Table 3, and/or Table 4, and/or Table 5, and/or Table 6, and/or Table 7, and/or Table 8, and/or Table 9, and/or Table 10.

Embodiment 75: The tolerogenic nanoparticle according to any one of embodiments 72-73, wherein said tolerogenic nanoparticle comprises a plurality of peptide epitopes selected from the peanut allergen epitopes shown in Table 2, and/or Table 3, and/or Table 4, and/or Table 5, and/or Table 6, and/or Table 7, and/or Table 8, and/or Table 9, and/or Table 10; or said tolerogenic nanoparticle comprises a nucleic acid encodes a plurality of peptide epitopes selected from the epitopes shown in Table 2, and/or Table 3, and/or Table 4, and/or Table 5, and/or Table 6, and/or Table 7, and/or Table 8, and/or Table 9, and/or Table 10.

Embodiment 76: The tolerogenic nanoparticle according to any one of embodiments 72-73, wherein said tolerogenic nanoparticle comprises 1, 2, 3, 4, 5, 6, 7, 8, 9, 10, 11, 12, 13, 14, or 15 epitopes selected from the peanut allergen epitopes shown in Table 10; or said tolerogenic nanoparticle comprises a nucleic acid that encodes 1, 2, 3, 4, 5, 6, 7, 8, 9, 10, 11, 12, 13, 14, or 15 epitopes selected from the epitopes shown in Table 10.

Embodiment 77: The tolerogenic nanoparticle according to any one of embodiments 72-73, wherein said tolerogenic nanoparticle comprises 1, 2, 3, 4, 5, 6, 7, 8, 9, or 10 epitopes selected from the peanut allergen epitopes in Tables 11 and 12 or said nucleic acid encodes 1, 2, 3, 4, 5, 6, 7, 8, 9, or 10 epitopes selected from the epitopes in Tables 11 and 12.

Embodiment 78: The tolerogenic nanoparticle according to any one of embodiments 1-70, wherein said peptide epitope comprises one or more dust mite peptide epitopes selected from the epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28.

Embodiment 79: The tolerogenic nanoparticle according to any one of embodiments 2-70, wherein tolerogenic nanoparticle comprises a nucleic acid that encodes a peptide epitope comprising one or more dust mite peptide epitopes selected from the epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28.

Embodiment 80: The tolerogenic nanoparticle according to any one of embodiments 78-79, wherein said tolerogenic nanoparticle comprises a single epitope selected from the dust mite epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28; or said tolerogenic nanoparticle comprises a nucleic acid encoding a single epitope selected from the dust mite epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28.

Embodiment 81: The tolerogenic nanoparticle according to any one of embodiments 78-79, wherein said tolerogenic nanoparticle comprises a plurality of epitopes selected from the dust mite epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28; or said tolerogenic nanoparticle comprises a nucleic acid encoding a plurality of epitopes selected from the dust mite epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28.

Embodiment 82: The tolerogenic nanoparticle of embodiment 81, wherein said epitope tolerogenic nanoparticle comprise at least 2, 3, 4, 5, 6, 7, 8, 9, 10, 11, 12, 13, 14, or 15 different dust mite epitopes, or said tolerogenic nanoparticle comprises a nucleic acid encoding at least 2, 3, 4, 5, 6, 7, 8, 9, 10, 11, 12, 13, 14, or 15 different epitopes.

Embodiment 83: The tolerogenic nanoparticle according to any one of embodiments 78-79, wherein said peptide epitope comprises 1, 2, 3, 4, 5, 6, 7, 8, 9, 10, 11, 12, 13, 14, or 15 epitopes selected from the dust mite epitopes shown in Table 28; or said nucleic acid encodes at least 1, 2, 3, 4, 5, 6, 7, 8, 9, 10, 11, 12, 13, 14, or 15 epitopes selected from the epitopes shown in Table 28.

Embodiment 84: The tolerogenic nanoparticle according to any one of embodiments 1-70, wherein said tolerogenic nanoparticle comprises one or more peptide epitopes from the adalimumab heavy chain and/or the adalimumab light chain, or said tolerogenic nanoparticle comprises a nucleic acid encoding one or more epitopes from the adalimumab heavy chain and/or the adalimumab light chain.

Embodiment 85: The tolerogenic nanoparticle of embodiment 84, wherein said tolerogenic nanoparticle comprises one or more adalimumab epitopes selected from the epitopes shown in Table 36, and/or Table 37, and/or Table 38, and/or Table 40, and/or Table 41, and/or Table 42, or said tolerogenic nanoparticle comprises a nucleic acid encoding one or more adalimumab epitopes selected from the epitopes shown in Table 36, and/or Table 37, and/or Table 38, and/or Table 40, and/or Table 41, and/or Table 42.

Embodiment 86: The tolerogenic nanoparticle according to any one of embodiments 84-85, wherein said tolerogenic nanoparticle comprises a single epitope selected from the adalimumab epitopes shown in Table 36, and/or Table 37, and/or Table 38, and/or Table 40, and/or Table 41, and/or Table 42; or tolerogenic nanoparticle comprises a nucleic acid that encodes a single peptide epitope selected from the epitopes shown in Table 36, and/or Table 37, and/or Table 38, and/or Table 40, and/or Table 41, and/or Table 42.

Embodiment 87: The tolerogenic nanoparticle according to any one of embodiments 84-85, wherein said tolerogenic nanoparticle comprises a plurality of peptide epitopes selected from the adalimumab epitopes shown in Table 36, and/or Table 37, and/or Table 38, and/or Table 40, and/or Table 41, and/or Table 42; or said tolerogenic nanoparticle comprises a nucleic acid that encodes a plurality of adalimumab peptide epitopes selected from the epitopes shown in Table 36, and/or Table 37, and/or Table 38, and/or Table 40, and/or Table 41, and/or Table 42.

Embodiment 88: The tolerogenic nanoparticle of embodiment 87, wherein said plurality of adalimumab peptide epitopes comprises at least 2, 3, 4, 5, 6, 7, 8, 9, 10, 11, or 12 different epitopes.

Embodiment 89: The tolerogenic nanoparticle according to any one of embodiments 1-70, wherein said tolerogenic nanoparticle comprises one or more peptide epitopes for the treatment or prophylaxis of diabetes, selected from the beta-cell autoantigen epitopes shown in Table 50, and/or Table 51, or said tolerogenic nanoparticle comprises a nucleic acid encoding one or more peptide epitopes for the treatment or prophylaxis of diabetes, selected from the beta-cell autoantigen epitopes shown in Table 50, and/or Table 51.

Embodiment 90: The tolerogenic nanoparticle of embodiments 89, wherein said tolerogenic nanoparticle comprises a single peptide epitope selected from the beta-cell autoantigen epitopes shown in Table 50, and/or Table 51, or a nucleic acid encoding a single peptide epitope selected from the selected from the epitopes shown in Table 50, and/or Table 51.

Embodiment 91: The tolerogenic nanoparticle of embodiments 89, wherein said tolerogenic nanoparticle comprises a plurality of peptide epitopes selected from the beta-cell autoantigen epitopes shown in Table 50, and/or Table 51, or a nucleic acid encoding a plurality of peptide epitopes selected from the selected from the epitopes shown in Table 50, and/or Table 51.

Embodiment 92: The tolerogenic nanoparticle of embodiment 91, wherein said plurality of peptide epitopes comprises at least 2, 3, 4, 5, 6, 7, 8, 9, 10, 11, or 12 different epitopes.

Embodiment 93: The tolerogenic nanoparticle according to any one of embodiments 1-70, wherein said tolerogenic nanoparticle comprises one or more histone autoantigen epitopes selected from the epitopes shown in Table 54, or said tolerogenic nanoparticle comprises a nucleic acid encoding one or more histone autoantigen epitopes selected from the epitopes shown in Table 54.

Embodiment 94: The tolerogenic nanoparticle of embodiments 93, wherein said tolerogenic nanoparticle comprises a single histone autoantigen epitope selected from the epitopes shown in Table 54, or a nucleic acid encoding a single histone auto-epitopes selected from the epitopes shown in Table 54.

Embodiment 95: The tolerogenic nanoparticle of embodiments 93, wherein said tolerogenic nanoparticle comprises a plurality of histone autoantigen epitopes selected from the epitopes shown in Table 54, or a nucleic acid encoding a plurality of histone autoantigen epitopes selected from the epitopes shown in Table 54.

Embodiment 96: The tolerogenic nanoparticle of embodiment 95, wherein said plurality of peptide epitopes comprises at least 2, 3, 4, 5, or 6 different epitopes.

Embodiment 97: The tolerogenic nanoparticle according to any one of embodiments 1-70, wherein said tolerogenic nanoparticle comprises one or more AAV peptide epitopes, selected from the epitopes shown in Table 59, and/or Table 60, and/or Table 61, or said tolerogenic nanoparticle comprises a nucleic acid encoding one or more AAV peptide epitopes, selected from the epitopes shown in Table 59, and/or Table 60, and/or Table 61.

Embodiment 98: The tolerogenic nanoparticle of embodiments 97, wherein said tolerogenic nanoparticle comprises a single peptide epitope selected from the AAV peptide epitopes shown in Table 59, and/or Table 60, and/or Table 61, or a nucleic acid encoding a single peptide epitope selected from the AAV peptide epitopes shown in Table 59, and/or Table 60, and/or Table 61.

Embodiment 99: The tolerogenic nanoparticle of embodiments 97, wherein said tolerogenic nanoparticle comprises a plurality of peptide epitopes selected from the AAV peptide epitopes shown in Table 59, and/or Table 60, and/or Table 61, or a nucleic acid encoding a plurality of AAV peptide epitopes selected from the epitopes shown in Table 59, and/or Table 60, and/or Table 61.

Embodiment 100: The tolerogenic nanoparticle of embodiment 99, wherein said plurality of peptide epitopes comprises at least 2, 3, 4, 5, 6, 7, 8, 9, 10, 11, or 12 different epitopes.

Embodiment 101: The tolerogenic nanoparticle according to any one of embodiments 1-100, wherein said nucleic acid that encodes one or more epitopes is an mRNA.

Embodiment 102: The tolerogenic nanoparticle of embodiment 101, wherein mRNA coding sequence is further modified by a non-natural RNA nucleobase N1-methylpseudouridine (m1Ψ).

Embodiment 103: The tolerogenic nanoparticle according to any one of embodiments 1-100, wherein said nucleic acid that encodes one or more epitopes is a DNA.

Embodiment 104: The tolerogenic nanoparticle according to any one of embodiments 101-103, wherein the N/P ratio of said nanoparticle ranges from 1:1 to about 10:1, or from about 2:1 to about 6:1, or from about 3:1 to about 5:1.

Embodiment 105: The tolerogenic nanoparticle of embodiment 104, wherein the N/P ratio of said nanoparticle is about 4:1.

Embodiment 106: The tolerogenic nanoparticle according to any one of embodiments 1-105, where when said peptide epitopes comprise a plurality of epitopes said peptide epitopes are joined by linker sequences; or where when said nucleic acid encodes a plurality of peptide epitopes said nucleic acid encodes linker sequence(s) joining said peptide epitopes.

Embodiment 107: The tolerogenic nanoparticle of embodiment 106, wherein said linker sequence(s) comprise a furin cleavage site.

Embodiment 108: The tolerogenic nanoparticle of embodiment 106, wherein said linker sequence(s) comprise wherein said linker sequences comprise a cathepsin cleavage site.

Embodiment 109: The tolerogenic nanoparticle of embodiment 106, wherein said linker comprises a Cathepsin L cleavage site.

Embodiment 110: The tolerogenic nanoparticle according to any one of embodiments 1-109, where said peptide epitope(s) are fused to an amino acid sequence that accesses trans-Golgi vesicles that transport type II MHC gene products to the cell surface; or said nucleic acid encodes peptide epitope(s) fused to an amino acid sequence that accesses trans-Golgi vesicles that transport type II MHC gene products to the cell surface.

Embodiment 111: The tolerogenic nanoparticle of embodiment 110, wherein said amino acid sequence comprises a transferrin receptor I transmembrane domain.

Embodiment 112: The tolerogenic nanoparticle of embodiment 110, wherein said amino acid sequence comprises a TfR 1-118 domain or the li-chain (114 aa).

Embodiment 113: The tolerogenic nanoparticle according to any one of embodiments 1-112, wherein said peptide epitope is modified to change one or more or all cysteines to serines or said nucleic acid encodes a peptide epitope in which one or more or all cysteines are replaced with serines.

Embodiment 114: A pharmacological formulation, said formulation comprising:
a tolerogenic nanoparticle according to any one of embodiments 1-113; and
a pharmaceutically acceptable carrier.

Embodiment 115: The pharmaceutical formulation of embodiment 114, wherein said formulation is a unit dosage formulation.

Embodiment 116: The pharmaceutical formulation according to any one of embodiments 114-115, wherein said formulation is formulated for administration via a route selected from the group consisting of oral administration, inhalation, nasal administration, rectal administration, intraperitoneal injection, intravascular injection, subcutaneous injection, transcutaneous administration, intrathecal administration and intramuscular injection.

Embodiment 117: A method for the treatment and/or prophylaxis of peanut allergy in a mammal, said method comprising: administering to said mammal an effective amount of a tolerogenic nanoparticle according to any one of embodiments 72-77.

Embodiment 118: A method for the treatment and/or prophylaxis of a dust mite allergy in a mammal, said method comprising: administering to said mammal an effective amount of a tolerogenic nanoparticle according to any one of embodiments 78-83.

Embodiment 119: A method for the suppressing or preventing an immune response to adalimumab in a mammal, said method comprising: administering to said mammal an effective amount of a tolerogenic nanoparticle according to any one of embodiments 84-88.

Embodiment 120: A method for the treatment and/or prophylaxis of type I diabetes in a mammal, said method comprising: administering to said mammal an effective amount of a tolerogenic nanoparticle according to any one of embodiments 89-92.

Embodiment 121: A method for the treatment and/or prophylaxis of lupus in a mammal, said method comprising: administering to said mammal an effective amount of a tolerogenic nanoparticle according to any one of embodiments 93-96.

Embodiment 122: A method for the suppressing or preventing an immune response to an AAV viral vector in a mammal, said method comprising: administering to said mammal an effective amount of a tolerogenic nanoparticle according to any one of embodiments 97-100.

Embodiment 123: The method of embodiment 122, wherein said tolerogenic nanoparticle is administered prior to administration of an AAV-based gene therapy vector to said subject.

Embodiment 124: The method of embodiment 122, wherein said tolerogenic nanoparticle is administered at the same time or overlapping time of administration of an AAV-based gene therapy vector to said subject.

Embodiment 125: The method according to any one of embodiments 117-124, wherein said mammal is a human.

Embodiment 126: The method according to any one of embodiments 117-124, wherein said mammal is a non-human mammal.

Embodiment 127: The method according to any one of embodiments 117-126, wherein said nanoparticle contains an immune modulator (e.g., an immune suppressant).

Embodiment 128: The method of embodiment 127, wherein said immune modulator comprises rapamycin or a rapamycin analog.

Embodiment 129: The method of embodiment 128, wherein said immune modulator comprises rapamycin (sirolimus).

Embodiment 130: The method of embodiment 128, wherein said immune modulator comprises a rapamycin analog selected from the group consisting of temsirolimus, everolimus, and ridaforolimus.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein, the phrase “a subject in need thereof” refers to a subject, as described infra, that suffers from, or is at risk for an autoimmune disorder and/or an allergic pathology, e.g., as described herein. Thus, for example, in certain embodiments the subject is a subject with an autoimmune disorder (e.g., type I diabetes, rheumatoid arthritis, lupus, etc.) or an allergic disease (e.g., asthma, and the like). In certain embodiments the methods described herein are prophylactic and the subject is one in whom an autoimmune disorder and/or an allergic disease is to be inhibited or prevented.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

A “nanoparticle” refers to a particle having an average size (e.g., diameter) below 1 μm. In certain embodiments the “nanoparticle” refers to a particle having an average size (e.g., diameter) below 500 nm.

The terms “lipid nanoparticle” or “lipidic nanoparticle” or “LNP” refers to spherical vesicles made up of ionizable lipids, which are positively charged pH (allowing RNA complexation and intracellular delivery) and neutral at physiological pH (promoting systemic biodistribution and reducing toxicity)

An “antigen” refers to any substance that can stimulate an immune response in the body and can react with the products of that response, that is, with specific antibodies or specifically sensitized T lymphocytes, or both.

The term “immune tolerance” refers to a state of unresponsiveness of the immune system to substances or tissue that have the capacity to elicit a suppressive or an anergic immune response to antigens that may also elicit a disease-inducing immune response to the same antigen in a given organism. The tolerogenic response can be induced by prior exposure to a specific antigen and differs mechanistically from the active immune response to foreign antigens.

The terms “effective fragment of an antigen” or “tolerogenic fragment of an antigen”, an “epitope”, or antigenic determinant are used interchangeably to refer to the part of an antigen that is recognized by the immune system, specifically by T-cells, antibodies, or B-cells. Although epitopes are usually non-self proteins, immunogenic fragments or epitopes can also be derived from endogenous host proteins and nucleosome components that could be recognized as antigenic determinants in the case of autoimmune disease.

The “stabilins” (e.g., stabilin-1, stabilin-2) are class H scavenger receptors that typically clear negatively charged and/or sulfated carbohydrate polymer components of the extracellular matrix from circulation (see, e.g., Murphy et al. (2005) Atherosclerosis, 182:1-15). They are large type I receptors composed of four fasciclin-1 domain clusters, four epidermal growth factor (EGF)/EGF-like clusters and one X-Link domain near the single transmembrane region. Although the human stabilin-1 and stabilin-2 extracellular portions of the receptors (>96% of the protein) are 55% homologous, the short intracellular domains are very diverse which contributes to differences in their location within the cell, cycling from the plasma membrane and downstream signaling activities.

“Apolipoprotein B” also called apolipoprotein B 100 or apoB is a protein that is involved in the metabolism of lipids and is a major protein constituent of lipoproteins such as very-low-density lipoprotein (VLDL) and low-density lipoprotein (LDL, the “bad cholesterol”). The amino acid sequence of apoB is well known to those of skill in the art (see, e.g., Chen et al. (1986) J. Biol. Chem., 261 (28): 12918-12921).

The term “mannan” refers to a plant polysaccharide that is a linear polymer of the sugar mannose. Plant mannans have β (1-4) glycoside linkages. In certain embodiments mannan can also refer to a cell wall polysaccharide found, inter alia, in yeasts. This type of mannan has an a (1-6) linked backbone and a (1-2) and a (1-3) linked branches. It is serologically similar to structures found on mammalian glycoproteins.

### BRIEF DESCRIPTION OF THE DRAWINGS

FIG. 1 schematically illustrates certain ionizable lipids for inclusion in hybrid biocompatible polymer/lipid tolerogenic nanoparticles and/or for formulation of lipidic tolerogenic nanoparticles.

FIG. 2 schematically illustrates the approach for making a mRNA construct that encodes for one or more tolerogenic T cell epitope(s) and can be incorporated into a nucleic acid delivery nanoparticle platform. mRNA is RNA that carries information from DNA to the ribosome, the site of protein synthesis (translation) within a cell.

FIG. 3 schematically illustrates the basic structural units in a mRNA construct for successful expression of antigenic proteins or peptides in cells, organs and injection sites of tolerogenic nanoparticles. The coding region (the part that is translated) has to contain start and stop codons to indicate where translation by RNA polymerase has to begin and where to end. In addition, the, delivered mRNA strand, also has to contain independent 5′ and 3′ untranslated regions (UTRs). The 5′ UTR, acting as a leader sequence, is found on the 5′ side of the coding region and includes a sequence for ribosomal recognition, thereby assisting in the initiation of translation by this organelle. The 3′ UTR is found immediately following the translation stop codon. The 3′ UTR plays a critical role in translation termination as well as post-transcriptional modification. In addition to these non-translated regions, the mRNA construct that is encapsulated by the nanocarrier also needs to be endowed with a 5′ cap and a poly(A) tail. Both sequences are added post-transcriptionally. The role of the 5′cap as a modified guanine nucleotide is to protect the transcript from being broken down. The poly-a tail also makes the RNA molecule more stable and prevents its degradation.

FIG. 4 shows a fusion construct cDNA sequence (bottom sequence, SEQ ID NO: 1) comprising a sequence encoding for the human transferrin receptor 1 (1-118) (top sequence, SEQ ID NO:2) linked to ten human Ara h1 and 2 epitope encoding nucleotide sequences,, which also encode recognition sites for proteolytic cleavage of the multi-epitope peptide translation product. The transferrin receptor sequence is added upstream of the multi-epitope sequence to ensure that cytosolic expression of the multi-epitope peptide is directed to the MHC-II endosomal compartment for peptide cleavage and insertion into the MHC-II peptide grove.

FIG. 5 illustrates insertion of the mRNA coding sequence into pTNT plasmid vector. The plasmid vector, pTNT, contains a promoter that includes the viral T7 or SP6 RNA polymerase, that drives downstream expression of an mRNA construct of interest. Elements to stabilize the transcribed RNA are incorporated into the vector. These include untranslated sequences before (5′ UTR), and/or after (3′ UTR) the cDNA sequence encoding the gene of interest. The 3′ UTR also includes a synthetic poly(A) tail, which is important for efficient translation of the protein. The pTNT plasmid is illustrative of a number of such vectors that can be used for mRNA strand expression.

FIG. 6. Anti-drug antibodies (ADA) are responsible for therapeutic antibody drug failure and side effects. The examples shown are for antibodies that are being produced against the monoclonal antibody, adalimumab, which is frequently used for the treatment of rheumatologic disease and a variety of immune disorders The schematic shows that this therapeutic monoclonal antibody contain antigenic peptide complementary determining regions (CDR) of the H and L chains, which could induce the production of the interfering antibodies against these antigenic sequences under the instruction of T cells. The T-cells recognize the antigenic sequences in the CDR regions of the therapeutic antibodies.

FIG. 7, panels A-B, illustrates liver-targeted PLGA nanoparticles for the treatment of type I diabetes. Panel A) depicts the NOD-SCID diabetes model in mice, in which the animals become diabetic after adoptive transfer of transgenic BDC2.5 T-cells. Diabetogenic BDC2.5 CD4 T cells were activated in vitro for 4 days with anti-CD3/28. NOD.SCID mice were injected with 200,000 activated BDC2.5 T cells and TNP loaded with a control peptide or a peptide mimotope recognized by the T cells. Blood glucose was monitored and the mice were sacrificed after consecutive readings >250 mg/dL. This mouse model was used to investigate whether the delivery of a peptide mimotope in the recipient animals can induce a tolerogenic effect preventing the onset of diabetes by Treg generation at the site of targeted liver sinusoidal endothelial cells. An agonistic peptide (mimotope) for diabetogenic T cell clone BDC2.5, adoptively transferred to non-obese diabetic NOD mice. The transgenic TCR from BDC2.5 recognizes the mimotope leading to pancreatic islet damage in the recipient in OD mice. Panel B) Illustrates tolerogenic nanoparticles (NPs) bearing the ApoBP peptide CGGKLGRKRTL (SEQ ID NO:3)

FIG. 8, panels A-B, illustrates treatment outcome in the recipient animals to TNP administration. This demonstrates the prevention of diabetes development in the recipients by the particles delivering the specific antigenic peptide, compared to animals injected with nanoparticles delivering a control (scrambled) peptide. Panel B demonstrates that the response is dose dependent.

FIG. 9, panels A-C, illustrates the histology of the pancreas and immunophenotyping of adoptively transferred T cells. Treatment with the mimotope-delivering nanoparticles reduces T cell recruitment to the pancreas, with the cytotoxic T cells being directed to the spleen. Panel A) TNP treatment induced a shift in T cell localization to the spleen. Transferred BDC2.5 T cells accumulate in the spleen in TNP-treated mice. Panel B) Decreased T cell infiltration in the pancreas of TNP-treated mice. Panel C) Histology demonstrates a lack of insulitis in islets of normoglycemic TNP-treated mice.

FIG. 10 shows increased LAG3 expression on the surface of the T cells recruited to the pancreas in TNP-treated mice. LAG3 is an exhaustion marker, indicating that tolerogenic therapy may impact island cell damage by an additional mechanism that involves reducing the cytotoxic potential of the adoptive transfered T cells.

FIG. 11 illustrates the use of tolerogenic nanoparticles delivering a T-cell epitope, present in the p70 splicosome, to the liver, with the ability to alleviate nephritis in a mouse MPR/Irp lupus model. Proteinuria (defined as >100 mg/dl in two occasions one day apart) was measured using albustix strips. Six mrl/lpr mice mice were used in each group; p<0.01.

FIG. 12, panels A-D, illustrates synthesis and characterization of the LSEC-targeting PLGA NP platform for OVA delivery. Panel (A): Schematic showing particle surface decoration with mannan and ApoBP: (i) mannan (man) was either physically adsorbed to the particle surface or its hydroxyl-terminus used for covalent conjugation to the PGLA COOH-terminal groups; (ii) ApoBP was linked to the NP surface by a NAEM spacer, using a two-step conjugation process between the ApoBP cysteine tag and the NAEM maleimide group. ApoB peptide containing a GGC tag: CGGKLGRKRYLR (SEQ ID NO: 4). Panel (B): Scanning electron microscopy pictures to show NP morphology, in the presence of attached ligands. Panel (C): Fourier transform infrared spectra of the NAEM-conjugated NPs. Panel (D): 1H NMR spectra of the synthesized particles with and without the ApoBP attachment, showing the appearance of the newly conjugated peptide at 7 ppm.

FIG. 13, panels A-B, shows that NP pretreatment interferes in OVA-induced antibody responses in an OVA sensitization and inhalation challenged OT II mouse model. Panel (A): Outlines the experimental animal protocol. Six to eight week old OT II mice received IV injection of NP^{OT II}, NP^{OTI}, and NP^{OVA} to deliver 25 μg antigen in 500 μg particles per mouse on days 0 and 7. The animals were subsequently sensitized by two IP doses of OVA (10 μg/mouse) on days 14 and 21, prior to being exposed to aerosolized OVA inhalation (10 mg/mL) for 20 min on days 35-37. Animals were sacrificed for tissue harvesting and BALF on day 40. Panel (B): Serum anti-OVA IgE and IgG1 antibody titers were determined by ELISA.

FIG. 14 shows an illustrative scheme to explain various features of the tolerogenic nanoparticle platform in terms of its intended use to treat peanut anaphylaxis. Previous studies have demonstrated the encapsulation and delivery of whole OVA or an MHC-II bound OVA peptide by PLGA nanoparticles can be targeted to LSECs in the liver by surface attachment of an ApoB peptide sequence (Liu et al. (2019) supra; Liu et al. (2021) supra). This leads to particle uptake by the stabilin-1 receptor, which is expressed on LSECs (Sørensen et al. (2012) supra; Li et al. (2011) Am. J. Physiol. Gastrointest. Liver Physiol. 300 (1): G71-G81). LSECs occupy a large surface area in the liver, serving as the primary source of contact during blood flow towards the central vein in the liver lobules (Shetty et al. (2018) Nat. Rev. Gastroenterol. & Hepatol. 15 (9): 555-567; Thomson & Knolle (2010) Nat. Rev. Immunol. 10 (11): 753-766). The slow blood flow and pressure drop in the sinusoids allow intimate contact of particulate matter with the scavenger receptors expressed on the LSEC surface, including selectin-1, which is endocytosed by clathrin coated pits (Shetty et al. (2018) supra). The degradation of antigens in this endocytic compartment allows release of hydrolyzed peptides and epitopes to reticuloendothelial vesicles, carrying MHC-II molecules to the cell surface (Thomson & Knolle (2010) supra). When presented to the T-cell antigen receptor (TCR) on naïve CD4⁺CD25⁻T-cells, antigenic epitopes embedded in the MHC groove act to induce the differentiation of antigen-specific CD4⁺/CD25⁺/FoxP3⁺ regulatory T-cells (Tregs). The APC function of the LSECs is assisted by tolerogenic cytokines (IL-10, TGF-β) produced or recruited to the cell surface (Horst et al. (2016) supra; Thomson & Knolle (2010) supra; Horst et al. (2021) Cell. Mol. Immunol. 18 (1): 92-111). Subsequent Treg release and recruitment to the lung or splanchnic mucosal compartment have proven quite effective for suppressing allergic lung inflammation and anaphylaxis in response to OVA or OVA epitopes, both prophylactically or post-sensitization (Liu et al. (2019) supra; Liu et al. (2021) supra). The ability of Tregs to engage in an infectious tolerance pathway, includes mechanisms that involve cell-cell contact (e.g., with TH2, B-cells, mast cells), release of cellular suppressive factors/cytokines (IL-10. TGF-β), competition for cytokine binding (e.g. IL-2), and effects on cellular recruitment (Kyte & Doolittle (1982) J. Mol. Biol. 157 (1): 105-132).

FIG. 15, panels A-B, illustrates epitope selection and encapsulation in liver-targeting PLGA nanoparticles. Panel (A): Ara h 2 epitopes were selected based on the role of this protein as a major peanut allergen that is recognized by IgE antibodies in the majority of peanut allergic patients (Orgel & Kulis (2018) supra). Epitope selection was initially carried out using the IEDB resource to identify the amino acid (aa) sequences, predicted to interact with murine H2-IAb, H2-IEd and H2-IAd alleles, with high binding affinity (IC50 values <50 nM) (Step 1). Four distinct 15-mer epitopes were identified, as shown herein in Table 66. Since the IEDB does not allow loading of the H2-IAk and H2-IEk allele sequences, expressed in C3H/HeJ mice, independent search was performed to compare the aa homology of H2-IAb with H2-IAk alleles. This demonstrated 95.7% and 92.5% homologies of the α- and β-allele sequences, respectively. We also selected a peptide sequence that is predicted to interact with IgE, as a comparative control for the T-cell epitopes. Step 2 was to determine the encapsulation efficiency of epitopes in PLGA nanoparticles, using the sum of the aa hydropathy values (GRAVY) and assessment of peptide p1. Peptides with negative GRAVY values (indicative of peptide solubility) facilitate peptide encapsulation (Kyte & Doolittle (1982) J. Mol. Biol. 157 (1): 105-132), leading to the selection of Epitope #4 and the IgE binding sequence for the synthesis of tolerogenic nanoparticles, as described in the Online Data section (FIG. 20, panel B). Two additional particle batches were synthesized to encapsulate purified Ara h2 or CPPE. Panel (B): SEM showing the morphology and size distribution of the synthesized PLGA nanoparticles.

FIG. 16, panels A-C, illustrates nanoparticle-induced Treg generation. Panel A) The experimental protocol was to use a one-time IV injection of a particle dose of 500 μg, which delivers 4 μg purified Ara h2 or epitope #4, into 6-8 week old C3H/HeJ mice (n=4). The animals were sacrificed 7 days later. Spleens were collected and single-cell suspensions prepared for Treg isolation, as described in methods. Panel (B): ELISPOT colony counts (number of spots per 3×10⁵ CD4⁺/CD25⁺ T-cells per well) were used to quantify the frequency of TGF-β producing cell clusters. The number of spots were quantified under a dissecting microscope, in addition to the capture of visual images with an ImmunoSpot Analyzer (Cleveland, OH). Panel C): Flow cytometry analysis of CD4⁺CD25⁺FoxP3⁺ Tregs, as determined by intracellular staining of Foxp3 and the use of Flowjo software (Ashland, OR). The software was used to calculate both the % positive cells as well as mean fluorescence intensity.

FIG. 17, panels A-G, illustrates the effect of prophylactic administration of tolerogenic PLGA nanoparticles on anaphylaxis induction by a crude peanut protein extract (CPPE). Panel (A): Experimental protocol outline, using C3H/HeJ mice (n=6) for IV NP injection on two occasions, 7 days apart. The particle dosimetry is discussed in Methods. Animal sensitization commenced a week later and was carried out at weekly intervals for four weeks, using CPPE and cholera toxin for oral gavage, at doses described in Methods. On week 6, mice were injected with 200 μg CPP into the peritoneal cavity to trigger an immediate anaphylactic response. Anaphylaxis was monitored by recording rectal temperatures every 10˜20 min, in addition to using three independent observers to record anaphylaxis scores. The scoring criteria were as follows: 0=no symptoms; 1=scratching and rubbing of the nose and head; 2=puffiness around the eyes and mouth, diarrhea, pilar erecti, reduced activity, and/or decreased activity with an increased rate of breathing; 3=wheezing, labored respiration, and cyanosis around the mouth and the tail; 4=no activity after prodding or tremor and convulsion; 5=death. Panel (B): Core body temperature assessment for 120 min. Panel (C): Anaphylaxis scoring at 60 min post-challenge (maximum temperature drop). Panels (D-G): Assessment of antigen-specific IgE and mast cell protease levels in the serum, as well as cytokine release in the peritoneal fluid, using ELISA.

FIG. 18, panels A-E, illustrates the post-treatment efficacy of the tolerogenic PLGA nanoparticles in suppressing the anaphylactic response. Panel (A:) Outline of the experimental animal protocol. The animals were sensitized by oral gavage as described in FIG. 17. On weeks 4 and 5, C3H/HeJ mice received IV injection of the tolerogenic nanoparticles. Mice received peritoneal injection with 200 μg CPP on week 6. Body temperatures and anaphylaxis scoring were performed as described. Panel (B): Core body temperature, as determined by a rectal probe. Panel (C): Anaphylaxis scores at 60 min. Panel (D): Serum mMCPT-1 level determined by ELISA. Panel E): TGF-β levels in the peritoneal lavage fluid.

FIG. 19, panels A-D, shows the duration of the tolerogenic effect, as determined by prophylactic particle administration. Panel A) Experimental design showing that PLGA nanoparticles were injected at three different time points prior to the onset of sensitization, as described in FIG. 16. After the challenge, the animal response parameters including core body temperature (panel B), anaphylactic scores (panel C), as well as the MCPT-1 release (panel D), assessment of antibody titers, MCPT-1 release and cytokine levels were performed as described in FIG. 17.

FIG. 20, panels A-B, outlines the establishment of the tolerogenic nanoparticle platform for treatment of peanut allergy and anaphylaxis. Panel A) The experimental design of the using the tolerogenic nanoparticle platform to deliver antigenic cargo to LSECs in the liver. Panel (B) depicts the synthesis of the poly(lactic-co-glycolic acid) nanoparticle nanocarrier, including the assessment of cargo loading of the epitopes, and recombinant protein, using a w/o/w double emulsion method combined with solvent removal.

FIG. 21, panels A-D, shows additional details of the anaphylaxis, serological and late-phase cytokine responses in the prophylactic animal model described in FIG. 17. Panel (A) Anaphylaxis score at the time points of 30 and 100 min after challenge. Panels (B) and (C) Peanut-specific IgG1, IgG and IgG2b antibody titers. Panel D) IL-5 level in the peritoneal lavage fluid.

FIG. 22, panels A-D, show additional data collected during the execution of the post-sensitization anaphylaxis intervention in FIG. 18. Panel A) Anaphylaxis score at 30 and 100 min after CPPE challenge. Panels B and C) Peanut-specific IgE, IgG1, IgG and IgG2b antibody titers. Panel D) IL-4 and IL-5 levels in the peritoneal lavage fluid.

FIG. 23, panels A-E, shows additional data collection during execution of the experiment in FIG. 19, looking at the impact of different time intervals of prophylactic treatment. Panel A) Anaphylaxis scores at 30 and 100 min after CPPE challenge. Panels B and C) Peanut-specific IgE, IgG1, IgG and IgG2b antibody titers. Panels D and E) IL-4, TGF-β, and IL-5, level in the peritoneal lavage fluid.

FIG. 24, panels A-C, illustrates the development of a liver targeting cationic lipid nanoparticles (LNP), delivering mRNA for the expression of green fluorescence protein (GFP). Panel A) Calculation and preparation of the aqueous (contains the GFP-mRNA) and organic (lipid constituents) phases for blending in a Microfluidics mixer. For proof-of-principle purposes, we selected GFP and polyA as RNA cargo examples to develop our in-house LNP platforms. GFP-mRNA and polyA encapsulating LNPs (GFP-mRNA LNPs, PolyA LNPs) were synthesized through the mixing of the ionizable cationic lipid, i.e. DLin-MC3-DMA, with DSPC, cholesterol and DMG-PEG-2k, dissolved in ethanol in the molar ratios of 50, 10, 38.5, 1.5, while the mRNA (GFP-mRNA or PolyA) components were diluted in Rnase-free 100 mM Sodium Acetate Buffer (pH 4.0). Briefly, the lipid compositions of DLin-MC3-DMA, DSPC, cholesterol and DMG-PEG-2k were dissolved in ethanol stock solutions at 10, 20, 10 and 20 mg/mL, respectively. To prepare a 2.2 mL LNP batch, an appropriate amount (listed in panel A lower right table) of each of the lipid stock solution was mixed in the ethanol phase to provide a 0.55 mL lipid mix, designated as the Organic Phase. The Aqueous Phase was prepared by dissolving 0.2 mg GFP-mRNA or PolyA in 1.4438 mL sodium acetate buffer. PolyA encapsulated LNPs (PolyA LNPs) were synthesized as a negative control for GFP expression. The final concentration of RNA in the Aqueous Phase was calculated to be 0.12 mg/mL; the final concentration of lipid mix was calculated to be 8.72 mM. The chemical structures of the four lipids are listed in panel B) Following calculation and preparation of the Aqueous and Organic Phases, a microfluidics mixer (NanoAssemblr Benchtop, developed by Precision NanoSystems Inc.) was used to synthesize our initial batch of mRNA delivering LNPs. Several important parameters in the NanoAssemblr Benchtop Software had to be calculated and inserted into in the table appearing in the upper right panel of panel B. These critical parameters include the Flow Rate Ratio at 3:1 (aqueous: organic), total flow rate of 12 mL/min, and N/P ratio at 4:1, etc. After triggering sample, mixing, the flow through samples were collected and purified by ultracentrifugation to remove the excess ethanol, before the reconstitution to the desired volume. Briefly, the primary LNP samples were purified by filtration of the 40 x diluted sample in sterile Ca²⁺- and Mg²⁺-free PBS using an Amicon® Ultra-15 centrifugal filtration tube (10 k) at 2000 ×g for 30 minutes at 20° C. Panel C) The LNPs samples were imaged by cryogenic electron microscopy (Cryo-EM) to investigate its nano-precipitated core structure. These cryo-EM images clearly demonstrate the mRNA encapsulation, in which the nucleic acid binding to the cationic lipid leads to the formation of a multilayered core structure. Taken together, the encapsulation efficiency (EE %) and cryo-TEM evidence show that the mRNA-LNPs form nanoparticles with encapsulated mRNA that is protected from the external medium. Scale bars represent 100 nm.

FIG. 25, panels A-B, shows GFP expression in liver sinusoidal endothelial cells (LSECs) exposed to GFP-mRNA LNPs. Confocal imaging (Leica SP8-MD confocal microscope) was performed by incubating the particles with liver sinusoidal endothelial cells (LSECs) at 2 μg/mL for 24 hours. Confocal imaging showed that the GFP-mRNA encapsulated in the LNPs was successfully expressed as GFP in LSEC cells (panel A), in contrast to polyA-LNPs not yielding any fluorescence (panel B). The green fluorescence color represents the green fluorescent protein expression in LSEC.

FIG. 26, panels A-C, illustrates LNP in vivo biodistribution to the liver after intravenous injection. Panel A) In order to visualize expression of GFP-mRNA in organs impacted by LNP biodistribution, it was necessary to synthesize further formulated an infrared fluorescence-labeled GFP-mRNA LNP, DIR/GFP-mRNA LNP, by replacing 0.1% cholesterol in the lipid composition with DiOC18 (7) (‘DiR’), which is becoming brightly fluorescent when incorporated into membrane lipids. All other synthesis parameters were the same as outlined for GFP-mRNA LNPs. Panel B) The size and the surface potential of the DiR/GFP-mRNA LNP were determined by Zetasizer Nano ZS. The EE % of the DiR/GFP-mRNA LNP was 97.6%. The cryo-EM image confirmed a nanoprecipitated mRNA core structure. Six hours after i.v. injection of the DiR/GFP-mRNA LNP at a dose of 20\hat{ }g in each animal, a strong infrared fluorescence signal could be observed in the abdominal region by IVIS imaging. Following animal sacrifice, ex vivo IVIS imaging demonstrated that most of the particles were distributed to the liver, with the spleen also showing some particle uptake. Panel C) Following tissue sectioning, immunofluorescence staining was performed using an AlexaFluor488-conjugated goat anti-mouse GFP-antibody. The slides were scanned with an Aperio AT Turbo digital pathology scanner (Leica Biosystems) at 10* magnification. High-magnification images were obtained under the 40* objective. The upper left image in panel C shows intrahepatic GFP expression in a pattern reminiscent of liver lobules, with clear central veins (c.v.) and a sinusoidal structure. Scanning under higher magnification (upper right image) shows GFP expression that appears to involve hepatocytes, LSEC and possibly Kupffer cells. Similarly, the lower left image shows the structure of the spleen with clear differentiation of the red and white pulp regions. Noteworthy, the higher magnification image (lower right image) shows GFP expression in splenocytes in both the red and white pulp.

FIG. 27, panels A-C, illustrates the design of an MHC-II targeting mRNA coding region for Arah2 #4 epitope. Panel A) The coding region for Arah2 #4 epitope includes a Start codon (ATG), MHC class II Targeting signal (Invariant chain li₁₋₈₀), Arah2 #4 epitope codons (two repeats) and a Stop codon (TAA) (SEQ ID NO:1237). Epitope recognition by CD4+Tregs requires to be directed to the MHC-II endosomal compartment for surface expression on LSECs. Although this is accomplishable by expressing the epitope sequence downstream of the transferrin receptor (TfR 1-118), we chose the invariant (li) chain sequence (1-80) in this instance. Our design also included inserting a flanking sequence between the epitope repeats for demarcation of their boundaries. These sequences are chosen because they represent natural flanking amino acids that are recognized as processing sites for immune presentation. Upon entry of the codon sequence in GenSmart™ Codon Optimization (https://www.genscript.com/gensmart-free-gene-codon-optimization.html), we obtained a final sequence with a GC content of 53.17%. Panel B) As a control for the peanut epitope sequence, we also designed a MHC-II targeting mRNA strand for the expression of multiple epitopes that play a role in a different disease model, with these epitopes are involved in causing diabetes mellitus. It included a Start codon (ATG), MHC class II Targeting signal (Invariant chain li₁₋₈₀), 10 diabetes epitope codons (InsB₉₋₂₃, GAD₆₅₂₈₆₋₃₀₀, mimotope P79 (2.5), mimotope BDC2.5, ChgA, Hip2.5, IGRP206-214) and a Stop codon (TAA) (SEQ ID NO: 1238). Flanking sequences were inserted between the epitopes for the purpose of epitopes splicing, prior to MHC II recognition. The final GC content was 70.1%. Panel C) Schematic of the structural components of the codon-optimized mRNA construct synthesized by TriLink® for LNP delivery and expression of the Arah2 #4 epitope or string of diabetic epitopes in cells and the liver after IV injection. The mRNA constructs make use of a natural Cap 1 structure (CleanCap®) along with a poly(A) (120) tail. Following in vitro transcription, the mRNA constructs were processed by DNase and phosphatase treatment as well as silica membrane purification to obtain purified single-stranded mRNA at the expected size. The final products were packaged as a solution in 1 mM sodium citrate buffer at pH 6.4.

FIG. 28, panels A-E, illustrates construction and use of Arah2 #4 mRNA LNPs to induce Treg generation and IL-10 expression in vivo. Panel A) Following codon optimization and in vitro transcription, inhouse synthesis of Arah2 #4-mRNA LNPs and DM-mRNA LNPs were formulated using the NanoAssemblr Benchtop microfluidic instrument. The lipid compositions, parameter settings and the downstream processing are similar to the protocols used for constructing the GFP-mRNA LNPs. LNP characterization showed that the size of the Arah2 #4-mRNA LNPs and DM-mRNA LNPs were 108.3±0.3 nm and 102.9 ±1.0 nm, respectively. The encapsulation efficiency (EE %) were 92.0% and 90.1%, respectively. The cryo-EM image for the peanut epitope demonstrated synthesis of an LNP with nano-precipitated mRNA core structure. Scale bar represents 100 nm. mRNA-LNP-induced Treg induction. Panel B) Demonstration of the generation of regulatory T cells (Treg) in the spleen by flow cytometry. Seven-week-old C3H/HeJ mice received i.v. injection of 25 pg LNPs incorporating (i) Arah2 #4-mRNA or (ii) DM-mRNA, in addition to a positive control animals group receiving (iii) 500 pg PLGA NPs incorporating 4 μg of the Arah2 #4 epitope peptide. The animals were sacrificed 7 days after. The spleens were collected and filtered through a 70 pm cell strainers to obtain single cell suspensions. The cells were then stained to detect expression of FoxP3 transcription factor in the CD4⁺CD25⁺ cell population by flow cytometry, in addition to performing Elispot assays to detect IL-10 creation in the CD4⁺CD25⁺ T cells. Panel C) Histograms depicting the % of FOXP3+in CD4⁺CD25⁺ T cells by flow cytometry. The cells were stained using AlexaFluor 488 anti-CD4 antibody, PE anti-CD25 antibody, and APC anti-FoxP3 antibody, and analyzed in a BD LSRII (IMED) analytic flow cytometer. Flowjo software (Ashland, OR) was used to quantify the percentage and mean fluorescence intensity. Panel D) Single cell suspensions were used for isolating CD4⁺CD25⁺ T-cells, using the Regulatory T Cell Isolation Kit from Miltenyi Biotec, before performance of the IL-10 Elispot assay. The spots on each well were detected and analyzed using a dissecting microscope. Panel E) Statistic analysis of spots in different groups. (*p<0.05, **p<0.01, ***p<0.001).

FIG. 29, panels A-D, illustrates protection in an animal model for peanut-induced anaphylaxis. Panel A) Seven-week-old C3H/HeJ mice received i.v. particle injections to deliver 50 μg of Arah2 #4-mRNA or DM epitope-mRNA in each animal during weeks 0 and 1. The animals were subsequently sensitized by 200 uL PBS containing 4 mg crude peanut allergen extract (CPPE) and 10 ug cholera toxin by oral gavage once a week for 3 weeks. On week 5, mice were injected with 200 μL PBS containing 200 ug CPPE into the intraperitoneal cavity. The Animal's core body temperature was measured using a rectal thermometer probe and recorded every 15 min. Animals were also monitored to score the anaphylaxis severity according to the following criteria: 0=no symptoms; 1=scratching and rubbing of the nose and head; 2=puffiness around the eyes and mouth, diarrhea, pilar erecti, reduced activity, and/or decreased activity with the increased respiratory rate; 3=wheezing, labored respiration, and cyanosis around the mouth and the tail; 4=no activity after prodding or tremor and convulsion; 5=death. The treatment groups (n=6) in the experiment included (i) a control group without LNP pretreatment, sensitization, or challenge; (ii) no pretreatment before sensitization and challenge; and pretreatment with (iii) Arah2 #4-mRNA LNPs, and (iv) DM-mRNA LNPs. Panel B) Core body temperature measured by the rectal probe. Panel C) Anaphylaxis score at the 75 min mark, including the occurrence of animal death. Panel D) Serum levels of the mast cell protease, mMCPT-1, assessed by ELISA. The LNP-Arah2 #4 provided significant (but not total) protection against core temperature drop, symptom scores, and mast cell protease release compared to LNP-DM and sensitization/challenge groups. Regarding the occurrence of animal death in the course of anaphylaxis, LNP-Arah2 #4 reduced the death rate from 4 in the sensitization/challenge group to one death in the peanut epitope treated animals. Curiously, although animals receiving the DM-mRNA LNPs showed an exaggerated anaphylaxis score, no animal deaths occurred. These data show that the peanut mRNA-LNP is partially effective in suppressing the severe anaphylactic response in an ultrasensitive animal model. (*p<0.05, compared to Arah2 #4-mRNA LNPs; #p<0.05 compared to sensitization)

FIG. 30, panels A-D, shows schematics that illustrate mannose-decorated mRNA lipid nanoparticles to improve epitope delivery to LSECs for tolerization of a peanut anaphylaxis response. Panel A) In the overall scheme, we propose to develop a tolerogenic LNP platform for enhancing the delivery of allergen epitopes to LSECs by attachment of a surface ligand, similar to what has been done for ApoB peptide decorated PGLA nanoparticles. Panel B) Schematic to compare the structural differences of non-decorated with decorated LNP, by implementing the use of mannose, conjugation to a lipid component on the particle surface. Panel C) Mannose selection is based on the surface expression of endocytic receptors on LSECs. Panel D). The structure of commercially available DSPE-PEG2000-Mannose, which was used, to partially replace the DMG-PEG2000 in the LNP formulation described in Example 5.

FIG. 31, panels A-B, illustrates preparation and characterization of Mannose-decorated LNPs to achieve LSEC targeting. Panel A) LNP composition of decorated versus non-decorated particles and synthesis parameters during the use of the NanoAssemblr, as described in Example 5. Panel B) schematic display of the structure of the mRNA loaded LNP-Man, including lipid chemistry.

FIG. 32, panels A-C, illustrates cellular uptake and GFP expression in LSECs treated with decorated and non-decorated LNP. Panel A) Schematic to describe the approach to assessing LNP cellular uptake and GFP expression experiment. 5×105 LSECs were added into each well of 6-well plate for 24 h before cellular treatment with mGFP/LNP or mGFP/LNP-Man (RNA concentration: 0.25 ug/mL or 0.5 ug/mL) for a further 24 h. The cells were harvested for performance of flow cytometry analysis. Panel B) GFP expression in LSECs, showing slightly bigger shift in cells treated with decorated versus non-decorated particles. Panel C) Mean fluorescence intensity of LSECs after treatment with mGFP/LNP or mGFP/LNP-Man. (n=3, *p<0.05, **p<0.01). These results demonstrated that both decorated and non-decorated particles are taken up by LSECs, with significant increase in GFP expression in relation to the former particle uptake.

FIG. 33, panels A-B, illustrates competitive inhibition of Cellular GFP expression by mGFP/LNP-Man in the presence of an axis of PolyA/LNP-Man. Panel A) Shift in the intensity of GFP expression in LSECs, 24 hours after incubation with the exposure parameters shown in Table 73. Panel B) Assessment of GFP mean fluorescence intensity in LSECs in the different groups (n=3, ***p<0.001). The results show that PolyA/LNP-Man or the anti-CD206 antibody can inhibit GFP expression by mGFP/LNP-Man.

FIG. 34, panels A-B, illustrates the use of a red blood cell assay to simulate the impact pH on the LNP's ability to use its hexagonal lipid phase for endosome escape. Panel A) Pictures of murine red blood cells (2%) treated with LNP or LNP-Man at pH 7.4 or pH 5.5 for 2 h. Panel B) Supernatants were collected from the cells shown in panel A to assess hemoglobin release in a microplate reader at absorbance of 540 nm. The data were normalized to RBC treated with 0.1% of the detergent, Triton X-100 (n=3, ***p<0.001). The results show lack of hemolysis in RBC treated with LNP or LNP-Man at pH 7.4. In contrast, RBC were lysed when treated with both particle types at pH 5.5, in a dose-dependent manner. Thus, there is no evidence that mannose should interfere in endosomal delivery of mRNA for expression in the transduced cells.

FIG. 35, panels A-B, illustrates the in vivo biodistribution of LNP after intravenous injection. Panel A) In vivo and ex vivo distribution of DiR/mGFP/LNP or DiR/mGFP/LNP-Man, 6 h after i.v. injection. Panel B) Statistical analysis of the average DiR radiant efficiency in different organs (n=3, * P<0.05, N.S. No significant differences). The results show that both LNP and LNP-Man selectively accumulate in liver and spleen. There was a significant reduction in the irradiance fluorescence in the spleen of animals treated with decorated nanoparticles.

FIG. 36, panels A-C, illustrates intrahepatic GFP mRNA expression in relation to nuclear staining and the particle DiR label. Panel A) Fluorescence distribution in liver slices. (Scale bar is 200 μm). Panel B) Relative GFP intensity in images from panel A (n=3, **p<0.01). Panel C) Co-localization rate of GFP with LSECs from panel A (n=3, *p<0.05). Compared to DiR/mGFP/LNP, the abundance of GFP expression in the liver was higher for DiR/mGFP/LNP-Man than non-decorated particles. Calculation of the percentage of co-localization for decorated particles (29%) was higher than for non-decorated LNP (17%).

FIG. 37, panels A-E, shows that mAra h2 loaded LNP induce Treg generation and IL-10 expression in vivo. Panel A) Schematic to show the assessment of Treg generation and IL-10 expression by mAra h2/LNP, as described in Example 5. Panels B-D) Characterization details for mAra h2/LNP and mAra h2/LNP-Man (panel B), and size distributions (panels C and D). Panel E) Representative images from the IL-10 ELISpot assay. Panel F) Statistic analysis of spark density in the different groups. (n=3, **p<0.01, ***p<0.001, N.S. No significant difference). The data demonstrate a statistical significant increase in the number of IL-10 spots by the PLGA and LNP nanoparticles delivering mAra h2 peptide or mRNA. Moreover, the number of spots is statistically significantly increased by decorated versus non-decorated LNP.

### DETAILED DESCRIPTION

In various embodiments tolerogenic nanoparticles that can induce epitope-specific immune tolerance and uses of such tolerogenic nanoparticles are provided. As illustrated in Example 1, the engineering of a biodegradable polymeric poly(lactic-co-glycolic acid) (PLGA) nanocarrier for the selective delivery of the allergen, ovalbumin (OVA), to the liver was demonstrated. This was accomplished by developing a series of nanoparticles (NPs) in the 200-300 nm size range as well as decorating particle surfaces with ligands that target scavenger and/or mannose receptors on liver sinusoidal endothelial cells (LSECs). LSEC represents a major antigen-presenting cell type in the liver capable of generating regulatory T-cells (Tregs). As shown in Example 1, in vitro exposure of LSECs to NP^{OVA} induced abundant TGF-β, IL-4, and IL-10 production, which was further increased by surface ligands. Animal experiments showed that, in the chosen size range, NP^{OVA} was almost exclusively delivered to the liver, where the colocalization of fluorescent-labeled particles with LSECs could be seen to increase by surface ligand decoration. Moreover, prophylactic treatment with NP^{OVA} in OVA-sensitized and challenged animals (e.g., via aerosolized inhalation) could be seen to significantly suppress anti-OVA IgE responses, airway eosinophilia, and TH2 cytokine production in the BALF. The suppression of allergic airway inflammation was further enhanced by attachment of surface ligands, particularly for particles decorated with the ApoB peptide, which induced high levels of TGF-β production in the lung along with the appearance of Foxp3+Tregs. The ApoB-peptide-coated NPs could also interfere in allergic airway inflammation when delivered post-sensitization.

Similarly, as shown in Example 3, herein, a dominant Ara h 2 T-cell epitope was delivered to liver sinusoidal endothelial cells in C3H/HeJ mice, using a poly(lactide-co-glycolide acid) nanoparticle construct. The carrier was intravenously injected prior to and following oral sensitization to crude peanut protein extract (CPPE), with comparison making to encapsulated CPPE, purified Ara h 2, an IgE-binding peptide. Disease outcome was assessed by monitoring core body temperature, anaphylaxis scores, mast cell release and regulatory T-cell (Treg) generation.

Prophylactic as well as post-sensitization administration of the encapsulated Ara h 2 T-cell epitope was effective in eliminating anaphylactic manifestations, hypothermia and mast cell release. This was accompanied by decreased peanut-specific IgE levels and increased TGF-β release in peritoneal lavage fluid. The prophylactic impact was more comprehensive than post-sensitization intervention, remaining effective for two months. The purified protein had lesser effects, with the CPPE and the IgE-binding epitope lacking effect. ELISPOT assays confirmed the generation of antigen-specific FOXP3⁺ Tregs by the dominant T-cell epitope. Thus it was demonstrated that an Ara h 2 T-cell epitope, targeted to liver sinusoidal endothelial cells, was effective in generating Tregs, capable of interfering in the anaphylaxis response to CPPE.

These data indicate that LSEC targeting in the liver by tolerogenic NPs can be used for therapy of allergic airway disease, in addition to the potential of using their tolerogenic effects for other disease applications.

Accordingly, in various embodiments tolerogenic nanoparticles are provided that deliver one or more encapsulated epitopes for raising a tolerogenic immune response in the liver. It will be recognized that in certain embodiments the tolerogenic nanoparticles comprise a single protein fragment (e.g., a single epitope), while in other embodiments the tolerogenic nanoparticles comprise 2, 3, 4, 5, 6, 7, 8, 9, or 10 or more allergen epitope(s). In certain embodiments, the protein fragments can be provided as separate molecules/moieties or as peptide fragments, that are linked together, e.g., by specially designed peptide spacers. In the arena of vaccine development for infectious disease agents, the linking together of multiple epitopes into one peptide construct is also referred to as string-on-bead vaccines. As described herein, the same concept can be used for the encapsulation of linked allergen epitopes to induce tolerogenic responses by encapsulating nanoparticles.

In another embodiment the use of nanoparticles to generate tolerogenic immune responses against allergens or allergen epitopes may be adapted by constructing nanoparticles that encapsulate the corresponding nucleic acid sequences, encoding for the expression of one or a more T-cell epitopes. Accordingly, in certain embodiments the tolerogenic nanoparticles described herein comprise one or more nucleic acid constructs that encode proteins/peptides comprising one or more antigen(s)/epitopes to induce a suppressive immune response. In certain embodiments the nucleic acid comprises a DNA. In certain embodiments the nucleic acid comprises an mRNA.

The core principle behind mRNA vaccine technology is delivery of nucleic acid encoding sequences to host sinusoidal endothelial cells for the expression of protein(s) or peptides that can reach endosomal compartments where the proteolytically digested antigens can be presented by type II major histocompatibility (MHC) complexes. While endogenous antigens in the cytosol can be degraded by the 26 S proteosome to generate peptides that are imported into vesicles transporting Type I MHC complexes, special care needs to be taken to allow cytosolic antigens to gain access to trans-Golgi vesicles transporting type II MHC gene products to the cell surface (Vyas et al; Nature Reviews Immunology; 2009; 8:607-617). This aspect will be further described in the use of a transferrin receptor (aa 1-118) or an invariant (li)-chain domain (aa 1-214 sequences), capable of delivering cytosolic peptides to the MHC class II endosomal compartment. In certain embodiments the mRNA comprises one of two categories of mRNA constructs: 1) Non-replicating mRNA (NRM); and 2) Self-amplifying mRNA (SAM) constructs.

### Tolerogenic Nanoparticles

In various embodiments tolerogenic nanoparticle are provided for the delivery of one or more peptide epitopes or for the delivery of a nucleic acid encoding one or more peptide epitope(s).

### Nanoparticles for the Encapsulation and Delivery of Peptides (e.g., Peptide Epitope(s))

In certain embodiments, tolerogenic nanoparticles for the delivery of tolerogenic peptides (e.g., peptide epitopes) are provided where the nanoparticles comprise: (1) One or more biocompatible polymers; (2) One or more peptide epitope(s), each comprising an antigen fragment to which immune tolerance is to be induced by encapsulating tolerogenic nanoparticles. In various embodiments, these T-cell activating epitopes are selected from a panel of dominant peanut allergens, dominant house dust mite allergens, adalimumab T cell epitopes, autoimmune epitopes in pancreatic beta cells, histone epitope, and T-cell epitopes involved in immune reactions against adenovirus-associated vectors (AAV) T; and (3) A targeting moiety that binds to a liver sinusoidal endothelial cell (LSECs), where the targeting moiety is attached to the surface of said nanoparticle.

In certain embodiments the tolerogenic nanoparticles described herein incorporate a single antigen/epitope. In certain embodiments the tolerogenic nanoparticles described herein incorporate 2, 3, 4, 5, 6, 7, 8, 9, or 10, or more antigens in a single tolerogenic nanoparticle. Where multiple epitopes are present they can all be from the same antigen, or they can be from different antigens. In certain embodiments populations of tolerogenic nanoparticles are provided where the populations comprise different tolerogenic nanoparticles comprising different antigens. Thus, for example, in certain embodiments, the population contains 2, 3, 4, 5, 6, 7, 8, 9, or 10, or more different “types” of tolerogenic nanoparticles where each “type” of tolerogenic nanoparticle comprise a different antigen or antigens or comprises different epitope or epitopes. In various embodiments the tolerogenic nanoparticles are effective to induce partial or complete immune tolerance to the antigen(s), and/or to reduce an immune response to the antigen(s). This type of scenario is particularly important in the setting of autoimmune disease where the autoimmune disease process may begin by dominantly targeting a single antigenic epitope, which is later expanded to include additional epitopes on the same antigen by a mechanism known as epitope spreading. Thus, while a dominant epitope may be useful in pretreatment approaches to suppress disease onset, it is often required in the chronic autoimmune state that treatment may need to be administered to multiple epitopes or spreading epitopes.

It was also discovered that incorporation of an immune modulator (e.g., an immune inhibitor such as rapamycin and/or a rapamycin analog) can significantly enhance the activity of the tolerogenic nanoparticles described herein. Not only does the rapamycin offer the possibility of reprogramming the targeted antigen presenting cells in the liver but is also known to promote Fox P3 expression on Tregs, in addition to expanding Treg populations. Additional immunomodulators that can modify the quality of the Treg response, in addition to rapamycin, can be incorporated, e.g., as described below.

In various embodiments any of a number of biocompatible polymers can be used to form the immunogenic nanoparticles described herein. Such biocompatible polymers are well known to those of skill in the art and include but are not limited to polyesters (e.g., poly(lactic-co-glycolic acid), poly(glycolic acid), poly(lactic acid), poly(caprolactone), poly(butylene succinate), poly(trimethylene carbonate), poly(p-dioxanone), poly(butylene terephthalate), and the like), poly(ester amide) s (PEA) (see, e.g., Guerrero et al. (2015) J. Control Release. 211:105-117), polyurethanes and polyurethane copolymers (see, e.g., Cherng et al. (2013) Int. J. Pharmaceutics, 450 (1-2): 145-162), polyanhydrides (e.g., poly[bis (p-carboxyphenoxy) methane], poly[bis(hydroxyethyl) terephthalate-ethyl orthophosphorylate/terephthaloyl chloride], see, e.g., Chang et al. (1983) Biomaterials, 4 (2): 131-133), poly(ortho esters) (see, e.g., Nair et al. (2006) Adv. Biochem. Eng. Biotechnol. 102:47-90; Park et al. (2005) Molecules, 10:146-161), polyphosphoesters (e.g., polyphosphoesters Poly[bis(hydroxyethyl) terephthalate-ethyl orthophosphorylate/terephthaloyl chloride]), poly(alkyl cyanoacrylates) (e.g., poly(butyl cyanoacrylate), poly(β-hydroxyalkanoate) s, poly(hydroxybutyrate), poly(hydroxybutyrate-co-hydroxyvalerate, collagen, albumin, gluten, chitosan, hyaluronate, cellulose, alignate, and starch.

In certain embodiments the biocompatible polymer comprises one or more cationic polymers. It is noted that such cationic polymers are particular well suited for delivery of a nucleic acid (e.g., DNA or mRNA) and/or negatively charged proteins/peptides. Illustrative cationic polymers include, but are not limited to Poly-L-lysine (PLL), poly-ethylenimine (PEI; a branched cationic polymer, assisting in endosomal delivery), poly[(2-dimethylamino) ethyl methacrylate](pDMAEMA), polyamidoamine (PAMAM) dendrimers, biodegradable poly(β-amino ester) (PBAE) polymers, poly(amino-co-ester) (PACE)-based polymers (branched amino acids, assisting in endosomal delivery), and the like (see, e.g., Wahane et al. (2020) Molecules, 25:2866).

In certain embodiments the biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), and poly(hydroxybutyrate-co-hydroxyvalerate). In certain embodiments the biocompatible polymer comprises poly(lactic-co-glycolic acid) (PLGA).

In one illustrative, but non-limiting embodiment the biocompatible polymer comprises PLGA comprising a lactide/glycolide molar ratio ranging from about 45:55 to about 100:1. In certain embodiments the biocompatible polymer comprises PLGA comprising a lactide/glycolide molar ratio of about 50:50. In certain embodiments the biocompatible polymer (e.g., PLGA) incorporates polyethylene glycol (PEG), e.g., about 8% up to about 20% e.g., ˜2kDA or ˜5 kDA PEG.

In certain embodiments the nanoparticles (without attached ligand(s)) are of a size effective or preferred for phagocytic uptake by macrophages and/or dendritic cells. It has been reported in the literature that antigen-presenting cells (APCs) can phagocytose particles ranging in size from about 50 nm up to about 3 μm. Additionally, the fabrication of biocompatible polymer (e.g., PLGA) nanospheres ranging in size from nanometer to micrometer size has been described (see, e.g., Swider, et al. (2018) Acta Biomaterialia, 73:38-51). Accordingly, in various embodiments, immunogenic nanoparticles ranging in size (e.g., average or median diameter) from about 50 nm up to about 3 μm are contemplated. In certain embodiments the nanoparticles range in size from about 50 nm, or from about 75 nm, or from about 100 nm, or from about 125 nm, or from about 150 nm, or from about 200 nm, or from about 250 nm, or from about 300 nm, or from about 350 nm, or from about 400 nm, or from about 450 nm, or from about 500 nm up to about 3 μm, or up to about 2.75 μm, or up to about 2.5 μm, or up to about 2.0 μm, or up to about 1.75 μm, or up to about 1.50 μm, or up to about 1.25 μm, or up to about 1 μm, or up to about 900 nm, or up to about 800 nm. In certain illustrative, but non-limiting embodiments, the nanoparticles have a mean or median size (e.g., diameter) that ranges in size from about 500 to about 800 nm.

In certain embodiments the nanoparticle may be entirely composed of a biocompatible polymer (e.g., PLGA), while in other embodiments, the nanoparticle may also comprise one or more lipids. In certain embodiments the lipid(s) include lipid components, capable of activating an immunological “danger signal” and/or binding to a nucleic acid. Illustrative lipids include but are not limited to didodecyldimethylammonium bromide (DDAB), and 1,2-dioleoyloxy-3-trimethylammonium propane chloride (DOTAP). DOTAP is cationic lipid that can bind mRNA and nuclei acids, in addition to delivering dangers signals. Other suitable lipids include, but are not limited to cationic lipids (e.g., cationic lipids used in cationic lipid nanoparticles for Covid S protein mRNA delivery) which include, but are not limited to DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino) propyl (12Z,15Z)-3-[(9Z,12Z)-octadeca-9,12-dien-1-yl]henicosa-12,15-dienoate (DMAP-BLP), (2Z)-non-2-en-1-yl 10-[(Z)-(1-methylpiperidin-4 yl) carbonyloxy]nonadecanoate (L101), and the like. In certain embodiments, inclusion of lipid can also be used to fine-tune the particle size and/or charge.

In certain embodiments the lipid comprises up to 25%, [molar percentage (excluding viral proteins or fragment(s) thereof)] of the nanoparticle. In certain embodiments the lipid comprises about 1%, or about 2%, or about 3%, or about 4%, or about 5%, or about 6%, or about 7% up to about 25%, or up to about 20%, or up to about 15%, or up to about 10% (molar percentage (excluding viral proteins or fragment(s) thereof)) of the nanoparticle.

Methods of making polymeric nanoparticles are well known to those of skill in the art (see, e.g., Marin et al. (2013) Int. J. Nanomed., 8:3071-3091, and references therein).

In certain embodiments the antigen(s) are encapsulated within the nanoparticle, and this can readily be accomplished by combining the antigen(s) with the biocompatible polymer during nanoparticle synthesis. In certain embodiments the antigen(s) are covalently attached to the surface of the nanoparticle, e.g., by adsorption or by coupling directly or using a linker.

### Nanoparticles for Carrying Nucleic Acid(s) Encoding One or More Peptides (e.g., Peptide Epitope(s))

In certain embodiments, tolerogenic nanoparticles for the delivery of one or more nucleic acid(s) (e.g., DNA or mRNA) encoding tolerogenic peptides (e.g., peptide epitopes) are provided where the nanoparticles comprise: (1) A nanoparticle comprising one or more lipid(s) and/or a one or more biocompatible polymer(s); (2) a nucleic acid encoding a peptide epitope comprising an antigen or antigen fragment to which immune tolerance is to be induced by administration of said tolerogenic nanoparticle to a mammal, where the peptide epitope comprises an epitope selected from the group consisting an epitope from a peanut allergen, an epitope from a house dust mite allergen, an adalimumab T cell epitope, a pancreatic beta cell epitope, a histone epitope, and a viral vector T cell epitope, where said nucleic acid is encapsulated within or attached to the surface of the nanoparticle; where the nanoparticle comprises or consists of lipids that accumulate in the liver and/or the nanoparticle comprises (3) A targeting moiety attached to the surface of said nanoparticle where said targeting moiety binds to a liver cell (e.g., an LSEC).

In certain embodiments the tolerogenic nanoparticles described herein incorporate a nucleic acid encoding a single antigen/epitope. In certain embodiments the tolerogenic nanoparticles described herein incorporate a nucleic acid encoding 2, 3, 4, 5, 6, 7, 8, 9, or 10, or more epitopes. Where multiple epitopes are encoded, they can all be from the same antigen, or they can be from different antigens. In certain embodiments populations of tolerogenic nanoparticles are provided where the populations comprise different tolerogenic nanoparticles comprising nucleic acids encoding different antigens. Thus, for example, in certain embodiments, the population contains 2, 3, 4, 5, 6, 7, 8, 9, or 10, or more different “types” of tolerogenic nanoparticles where each “type” of tolerogenic nanoparticle comprise a nucleic acid encoding different antigen or antigens or comprises different epitope or epitopes. In various embodiments the tolerogenic nanoparticles are effective to induce partial or complete immune tolerance to the antigen(s), and/or to reduce an immune response to the antigen(s). This type of scenario is particularly important in the setting of autoimmune disease where the disease process may begin by dominantly targeting a single antigenic epitope, which is later expanded to include additional epitopes on the same antigen by a mechanism known as epitope spreading. Thus, while a dominant epitope may be useful in pretreatment approaches to suppress disease onset, it is often required in the chronic autoimmune state that treatment may need to administer multiple epitopes to account for epitope spreading.

It is also believed that incorporation of an immune modulator (e.g., an immune inhibitor such as rapamycin and/or a rapamycin analog) can significantly enhance the activity of the tolerogenic nanoparticles described herein. Not only does the rapamycin offer the possibility of reprogramming the targeted antigen presenting cells in the liver but is also known to promote Fox P3 expression on Tregs, in addition to expanding Treg populations. Additional immunomodulators that can modify the quality of the Treg response, in addition to rapamycin, can be incorporated, e.g., as described below.

In certain embodiments the tolerogenic nanoparticles comprising a nucleic acid (e.g., a DNA, an mRNA) can comprise one or more biocompatible polymers and optionally incorporate a lipid (e.g., a cationic lipid), while in certain other embodiments, the the tolerogenic nanoparticles comprising a nucleic acid (e.g., a DNA, an mRNA) can exclude biocompatible polymers and thererby provide nanoparticles comprising substantially or all lipids. Such nanoparticle may be designated lipid (or lipidic, or lipidoic) nanoparticles (LNPs).

Nucleic acid-based (e.g., mRNA-based) nanoparticles provide promising new leads for immunotherapy applications in the fields of cancer and infectious disease, as exemplified by the recent Covid 19 pandemic. Among these, lipid-based mRNA nanoparticles comprising ionizable lipids are gaining increasing attention as versatile technologies for delivering nucleic acids for a variety of applications. Such lipid-based nanoparticles can be fine tuned towards a given application, and have proven potential for successful development up to clinical practice (see, e.g., Dammes & Peer (2020) Trends in Pharmacological Sciences, 41:665-775; Uebbing et al. (2020) Langmuir, 36:13331-13341: Siewert et al. (2020) Cells, 9:2034; Kranz et al. (2016) Nature, 534:396). It has now been been demonstrated that the transfection efficacy of RNA lipid nanoparticles depends on the pKa value of the ionizable lipid used for particle formation (see, e.g., Uebbing et al. (2020) Langmuir, 36:13331-13341; Kauffman et al. (2015) Nano Lett. 15:7300-7306). More specifically, the cationic charge that these particles develop under acidic pH conditions is important for endosomal processing and mRNA release (Nel & Miller (2021) ACS Nano 15:5793-5818). In addition to pKa, the fusogenicity of different lipids, which results from their ability to form the reversed hexagonal phase Hn, has an influence in facilitating endosomal escape (see, e.g., Cullis et al. (2-017) Mol. Ther. 25:1467-1475: Heyes et al. (2005) J. Control Release, 107:276-287). Both of these lipid properties, which are a result of the headgroup structure and lipid tail saturation, are important factors to be kept in mind when choosing lipids for mRNA lipoplex formulation.

Unlike most other tissues, the liver does not exhibit impermeable basal lamina. Hence, in the absence of interfering mechanisms such as aggregation or protein binding, the majority of lipid nanoparticles (LNPs) can exhibit rapid passive liver accumulation following systemic administration (see, e.g., Jacobs et al. (2010) Am. J. Pathol. 176:14-21). Passive targeting of liver cells is mainly determined by the diameter of the liver sinusoidal fenestrae enabling selectivity towards liver sinusoidal endothelial cells (LSECs) and Kupffer cells on one side (>100 nm) or hepatocytes and HSCs on the other side (<100 nm).

Due to the biocompatible and nontoxic characteristics of lipid-based delivery systems, they have shown the most advances for delivery of nucleic acids (e.g., gene therapy) in human clinical trials. Recently, the development of siRNA lipid nanoparticles (LNPs) has experienced a significant breakthrough, which reflected by the FDA approval of ONPATTRO®, the first LNP delivering siRNA for treating a liver-based rare genetic disease, TTR-mediated amyloidosis.

The composition of a lipid nanoparticles (LNP) plays a major role in particle size, surface properties, encapsulation efficiency and intercellular release. Additionally, it influences the uptake mediation of the LNP cargo to the target cell and eventually a specific cellular compartment.

Among these, lipid-based mRNA nanoparticles comprising ionizable lipids are gaining increasing attention as versatile technologies for fine-tuning toward a given application, with proven potential for successful development up to clinical practice (see, e.g., Dammes & Peer (2020) Trends in Pharmacological Sciences, 41:665-775; Uebbing et al. (2020) Langmuir, 36:13331-13341: Siewert et al. (2020) Cells, 9:2034; Kranz et al. (2016) Nature, 534:396). It has now been been demonstrated that the transfection efficacy of RNA lipid nanoparticles depends on the pKa value of the ionizable lipid used for particle formation (see, e.g., Uebbing et al. (2020) Langmuir, 36:13331-13341: Kauffman et al. (2015) Nano Lett. 15:7300-7306). More specifically, the cationic charge that these particles develop under acidic pH conditions is important for endosomal processing and mRNA release (Nel & Miller (2021) ACS Nano 15:5793-5818). In addition to pKa, the fusogenicity of different lipids, which results from their ability to form the reversed hexagonal phase Hn, has an influence in facilitating endosomal escape (see, e.g., Cullis et al. (2-017) Mol. Ther. 25:1467-1475: Heyes et al. (2005) J. Control Release, 107:276-287). Both of these lipid properties, which are a result of the headgroup structure and lipid tail saturation, are important factors to be kept in mind when choosing lipids for mRNA lipoplex formulation.

In certain illustrative, but non-limiting embodiments efficient hepatic targeting LNP formulations for gene delivery are comprised of an ionizable amino lipid, helper lipids, PEG-lipid and cholesterol. The most important component is often the ionizable cationic lipid, e.g., often with a pKa ˜6.5. When formulating nucleic acids with lipids into the LNPs in an acidic condition (e.g., pH ˜4), the ionizable lipid is positively charged to interact with the negatively charged nucleic acids to promote the loading into the LNPs. The pH of the formed LNPs can then be brought up to, e.g., pH 7.4, and in normal physiological conditions, the LNPs become neutral for reduced non-specific interaction with cells and blood components. When the LNPs are internalized by the target cell and enter into the acidic endo/lysosomal compartment (e.g., pH ˜5), the lipid becomes positively charged again, mediating binding with the negatively charged endolysosomal membrane (Maier et al. (2013) Mol. Ther. 21:1570-1578). We have also demonstrated that the custom designed nanoparticles described herrien are capable of membranolysis under acidic but not neutral pH conditions. This interaction leads to endolysosomal disruption and cytosolic release of the nucleic acid.

The incorporation of an ionizable aminolipid to deliver nucleic acids was first reported with 1,2-dioleoyl-3-dimethylaminopropane (DODAP) LNPs (Maurer et al. (2001) Biophys. J. 80:2310-2326). This formulation composed of cholesterol, distearoyl phosphatidylcholine (DSPC), PEG-Lipid and DODAP (pKa 6.6), encapsulated up to 70% of the oligonucleotide into 100 nm-sized LNPs (Semple et al. (2001) Bba-Biomembranes, 1510: 152-166). This significant finding has led to the synthesis of an ionizable lipid library that played a major role in the improvement of in vivo hepatic nucleic acid delivery (see, e.g., Maier et al. (2013) Mol. Ther. 21:1570-1578; Jayaraman et al. (2012) Angew. Chem. Int. Ed. Eng. 51:8529-8533). The lipid structure and pKa of the ionizable lipids were demonstrated to have a significant impact on the efficient delivery of siRNA to the hepatocytes (Semple et al. (2010) Nat. Biotechnol. 28:172-176). Semple et al. (Id.) evaluated the effect of ester-, alkoxy-, and ketal-linkers, between the head group and alkyl chain of 1,2-dilinoleoyl-3-dimethylaminopropane, 1,2-dilinoleyloxy-3-dimethyl aminopropane (DLin-DMA), and 2,2-dilinoleyl-4-dimethylaminomethyl-[1,3]-dioxolane (DLin-K-DMA), respectively. The observed potency of DLin-K-DMA lipid on the in vivo silencing of factor VII was 2.5-fold higher than the other tested lipids, indicating the superior effect of the ketal linker (Jayaraman et al. (2012) Angew. Chem. Int. Ed. Eng. 51:8529-8533). A further modification to the ketal linker by incorporating a single methylene group (2,2-dilinoleyl-4-(2-dimethylaminoethyl)-[1,3]-dioxolane, DLin-KC2-DMA) has shown a 4-fold higher activity over DLin-K-DMA. Additionally, the pKa of the ionizable lipid was found to be a key factor for the potency of siRNA-LNPs. Ultimately, the most potent LNPs that is used in the ONPATTROR formulation is based on (6Z,9Z,28Z,31Z)-heptatriaconta-6,9,28,31-tetraen-19-yl-4-(dimethylamino) butanoate (DLin-MC3-DMA) lipid with a pKa of 6.44. This lipid showed 10-fold higher potency than DLin-KC2-DMA (Jayaraman et al. (2012) Angew. Chem. Int. Ed. Eng. 51:8529-8533; Lin et al. (2014) Clin. Lipidol. 9:317-331). There have been other reports focusing on developing new ionizable lipids to improve nucleic acid delivery. For instance, multiple ionizable groups were introduced in these novel ionizable lipids to enhance the gene delivery efficiency including dicetylphosphate-tetraethylenepentaamine-based polycation liposomes for systemic siRNA delivery (Asai et al. (2100) Bioconjug. Chem. 22:429-435) and alkenyl amino alcohols LNPs for mRNA delivery (Fenton et al. (2016) Adv. Mater. 28:2939-2943). Additionally, chemical modification of ionizable lipids such as biodegradable linkers can also improve the overall gene delivery (Fenton et al. (2017) Adv. Mater. 29 (33). doi: 10.1002/adma.201606944).

In addition to an ionizable lipid, helper lipids can be incorporated into the LNPs to improve their stability. 1,2-dioleoyl-sn-glycero-3-phosphoethanolamine (DOPE) was one of the first phospholipids used to promote membrane fusion. Due to the unsaturated acyl chains and small head group, DOPE can adapt the hexagonal phase and interact with the endosomal membrane. This interaction facilitates the release of payload to the cytosol by disrupting the endosomal bilayer (Hafez et al. (2001) Adv. Drug Deliv. Rev. 47:139-148). DSPC is regarded as the most efficient helper lipid in the LNP formulation for siRNA delivery. In contrast to DOPE, DSPC has two saturated acyl chains and a large head group which results in a cylindrical geometry that plays a major role in maintaining the stability and structure of the LNPs outer layer (Kulkarni et al. (2018) ACS Nano, 12:4787-4795). Another major component of the LNPs is cholesterol that influences membrane permeability and fluidity. Incorporation of cholesterol in the LNP formulation can result in tight packaging, leading to enhanced stability (Coderch et al. (2000) J. Control. Release, 68:85-95).

In certain embodiments a PEG-lipid is included in the LNP formulation to reduce aggregation during the particle formation, to prolong the storage stability (Bao et al. (2013) Pharm. Res. 30:342-351) and can be important for the effective delivery of nucleic acid into the hepatocytes. Mui et al. (2013) Mol. Ther. 2: e139, studied the effect of the PEG-lipid acyl chain length on the in vivo pharmacokinetics and delivery efficiency of siRNA. The short acyl chain PEG-C14 was found to dissociate rapidly at a rate of 45% per hour, whereas the longer chain PEG-lipids, PEG-C16 and PEG-C18 diffused out of the LNPs at slower rates of 1.3 and 0.2% per hour, respectively. Approximately 55% of the injected PEG-C14 LNPs accumulated in the liver within 4 h and were mainly internalized by the hepatocytes. PEGylation of nanoparticles is often employed to reduce the opsonization, and the above findings suggest that adsorption of serum proteins to the siRNA-LNPs can play an important role in hepatocyte targeting.

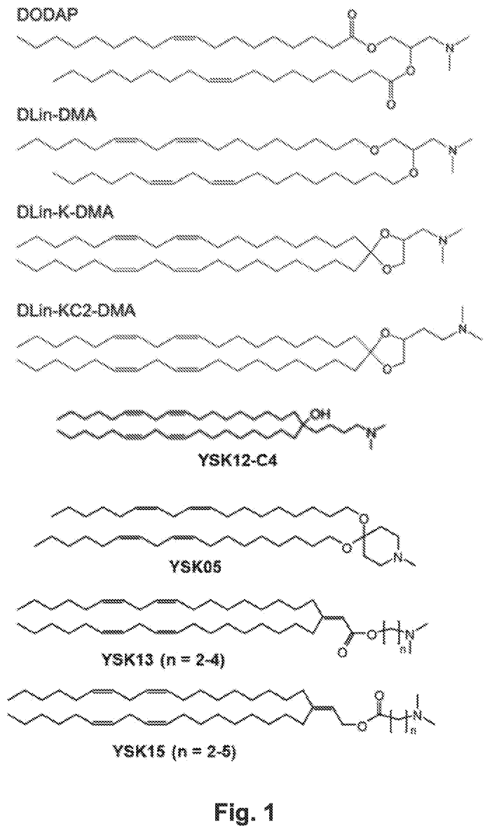

ONPATTROR (Alnylam) for treating TTR-mediated amyloidosis is the most clinically advanced siRNA-LNP product. Mutation in the TTR gene results in a build-up of amyloidosis protein in tissues and organs that can eventually cause cardiomyopathy or neuropathy. Alnylam has produced two generations of TTR-LNPs which involve the incorporation of different ionizable lipids. The formulation consisting of DSPC, cholesterol, PEG-1,2-Dimyristoyl-sn-glycerol (DMG) and either DLin-DMA (see, e.g., FIG. 1) in the first generation (ALN-TTR01) or DLin-MC3-DMA (see, e.g., FIG. 1) in the second generation (ALN-TTR02) (Wan et al. (2014) Drug Deliv. Transl. Res. 4:74-83). ALN-TTR02 showed improved efficacy in human patients, inducing up to 70% suppression of TTR at a dose of 0.15-0.5 mg/kg (Lin et al. (2014) Clin. Lipidol. 9:317-331; Coelho et al. (2013) N. Engl. J. Med. 369:819-829) (see, e.g., Lin et al. (2014) Clin. Lipidol. 9:317-331; Coelho et al. (2013) N. Engl. J. Med. 369:819-829). In August 2018, ALN-TTR02 known as ONPATTROR was approved by the FDA as a treatment for both mutant and non-mutant forms of TTR (Coelho et al. (2013) N. Engl. J. Med. 369:819-829; Garber (2018) Nat. Biotechnol. 36:777-778). ONPATTROR is also the first approved siRNA therapeutics and the first approved non-viral gene delivery system.

ALN-PCS is the latest development of siRNA-LNPs by Alnylam and has reached clinical trials in 2011. This formulation was developed to treat hypercholesterolemia by targeting proprotein convertase subtilisin/kexin type 9 (PCSK9). The ALN-PCS01 formulation is composed of lipidoid 98N12-5 (1) 4HCl, cholesterol, and mPEG2000-DMG at a molar ratio of 42:48:10. The phase I trial reported a dose-dependent silencing effect of PCSK9 with reductions of 84% PCSK9 protein and 50% LDL-cholesterol at the highest dose of 0.4 mg/kg (Id.). In the ALN-PCS02 formulation, lipidoid was replaced with DLin-MC3-DMA and an increase in potency in a phase I trial was reported with a 50% reduction of LDL-cholesterol at a low dose of 0.25 mg/kg (Alabi et al. (2012) Curr. Opin. Pharmacol. 12:427-433).

Sato et al. (2012) J. Control. Release, 163:267-276, developed a multifunctional envelope-type nanodevice (MEND) composed of a novel pH-sensitive cationic lipid for siRNA delivery. At first, MEND was incorporated into a peptide (PPD, GALA and shGALA)-based functionalizing device, which was later replaced with an ionizable lipid for improved gene silencing activity. YSK05 (see, e.g., FIG. 1) is one of their earlier synthesized ionizable lipids with one tertiary amine and two unsaturated acyl chains. This discovery was followed by the synthesis of various pH-sensitive lipids that showed liver-selective accumulation and efficient cytosolic delivery. MENDs were initially prepared using an ionizable lipid, helper lipid (DSPC, DOPE, 1,2-dioleoyl-sn-glycero-3-phosphocholine or 1-Palmitoyl-2-oleoyl-sn-glycero-3-phosphoethanolamine), cholesterol and PEG-DMG at a molar ratio of 50:10:40:3, and then optimized to a formulation of ionizable lipid: cholesterol: PEG-DMG at 70:30:3 (Sato et al. (2012) J. Control. Release, 163:267-276; Watanabe et al. (2014) Sci. Rep. 4:4750). The authors reported that the optimized formulation with no helper lipid and a higher ratio of the ionizable lipid resulted in enhanced endosomal escape due to an enhanced fusogenic property, and subsequently higher transfection efficiency (Hatakeyama et al. (2014) J. Control. Release, 173:43-50).

Sato et al. (2016) Mol. Ther. 24:788-795 investigated the effect of the lipid pKa on the hepatocyte targeting of MENDs. YSK05 and YSK13-C3 with pKa of 6.50 and 6.45, respectively, displayed significant delivery to the hepatocytes, and the YSK-13-C3 formulation showed four-fold higher gene silencing activity compared to YSK05 (Watanabe et al. (2014) Sci. Rep. 4:4750; Hatakeyama et al. (2014) J. Control. Release, 173:43-50; Yamamoto et al. (2016) J. Hepatol. 64:547-555). In contrast, lipids with a higher pKa (YSK13-C4 and YSK15-C4 with pKa 6.80 and 7.10, respectively) were found to be effectively taken up by the liver SECs, which can be utilized to specifically treat associated liver disorders (Shobaki et al. (2018) Int. J. Nanomedicine, 13:8395-8410). Lipase-resistant YSK05 (pKa 6.50) and YSK012-C4 (pKa 8.0) have used to prepare MENDs with a pKa of 7.15 to efficiently target the liver SECs (Shobaki et al. (2018) Int. J. Nanomedicine, 13:8395-8410; Warashina et al. (2016) J. Control. Release, 225:183-191).

The same group has also developed an ionizable SS-cleavable and pH-activated lipid-like material (ssPalmM) that contains a cleavable di-sulfide bond for liver-targeted delivery (Ukawa et al. (2014) Adv. Healthc. Mater. 3:1222-1229). This synthesized com-pound contains two myristoyl lipid chains and two ionizable tertiary amines linked through a disulfide bond. The ionizable amino groups are to promote nucleic acid loading and endosomal disruption and the disulfide bond is cleaved in the intracellular environment, which is reductive and GSH-rich, to facilitate nucleic acid release in the cytosol. Ukawa et al. (2014) Adv. Healthc. Mater. 3:1222-1229, reported a lipid envelope-type nanoparticle encapsulating pDNA for liver delivery, composed of ssPalmM, 1-octadecanoyl-2-(9Z-octadecenoyl)-sn-glycero-3-phosphocholine, cholesterol and distearoyl-rac-glycerol-PEG. This formulation demonstrated long-lasting hepatic gene expression in mice for 2 weeks with no toxicity or immunogenicity. It was then further developed to specifically deliver siRNA to the hepatocyte. Akita et al. (2015) ACS Biomater. Sci. Eng. 1:834-844, replaced the two myristoyl lipid chains in the ssPalmM with vitamin E (ssPalmE) and demonstrated improved gene silencing activity in mice by approximately 6-fold. The group reported that the incorporation of a highly fat-soluble vitamin E as a hydrophobic scaffold enhanced the hepatic delivery of siRNA.

To date, we have prepared mRNA encapsulating nanoparticles by using lipid film, ethanol injection, and microfluidic mixing methods, as described in protocols developed for the NANOASSEMBLR® Benchtop and illustrated herein, inter alia, in Example 5. For the lipid film method, we dissolve the lipids in required ratios in chloroform, followed by pipetting into glass vials. A thin film forms when the chloroform is evaporated in a rotary evaporator. The nucleic acid (e.g., mRNA) is diluted to the required concentration in 10 mM HEPES/EDTA buffer and then added to the lipid films. After vortexing the samples are left at room temperature overnight, before further vortexing and collecting the particles. For lipoplexes, we usean ethanol injection method. The lipids are dissolved in the required ratios in pure ethanol, and then injected into a glass vial containing mRNA diluted to the appropriate concentrations, using a handheld mixer. Microfluidic preparation is carried out in NanoAssemblr® Benchtop, using the mRNA Lipid Nanoparticles Robust low-volume production protocol from the manufacturer (Precision Nanosystems).

In addition to the use of cationic lipids, it is also known that mRNA can be delivered by cationic polymers, which can also be combined with cationic lipids for mRNA nanoparticle construction (see, e.g., Siewert et al. (2020) Cells, 9:2034). Methods of making polymeric nanoparticles are well known to those of skill in the art (see, e.g., Marin et al. (2013) Int. J. Nanomed., 8:3071-3091, and references therein), and can be adapted to deliver DNA or mRNA nucleic acid constructs. In various embodiments any of a number of biocompatible polymers can be used to form the tolerogenic nanoparticles described herein. Such biocompatible polymers are well known to those of skill in the art and include but are not limited to polyesters (e.g., poly(lactic-co-glycolic acid), poly(glycolic acid), poly(lactic acid), poly(caprolactone), poly(butylene succinate), poly(trimethylene carbonate), poly(p-dioxanone), poly(butylene terephthalate), and the like), poly(ester amide) s (PEA) (see, e.g., Guerrero et al. (2015) J. Control Release. 211:105-117), polyurethanes and polyurethane copolymers (see, e.g., Cherng et al. (2013) Int. J. Pharmaceutics, 450 (1-2): 145-162), polyanhydrides (e.g., poly[bis (p-carboxyphenoxy) methane], poly[bis(hydroxyethyl) terephthalate-ethylortho-phosphorylate/terephthaloyl chloride], see, e.g., Chang et al. (1983) Biomaterials, 4 (2): 131-133), poly(ortho esters) (see, e.g., Nair et al. (2006) Adv. Biochem. Eng. Biotechnol. 102:47-90; Park et al. (2005) Molecules, 10:146-161), polyphosphoesters (e.g., polyphosphoesters poly[bis(hydroxyethyl) terephthalate-ethyl orthophosphorylate/terephthaloyl chloride]), poly(alkyl cyanoacrylates) (e.g., poly(butyl cyanoacrylate), poly(β-hydroxyalkanoate) s, poly(hydroxybutyrate), poly(hydroxybutyrate-co-hydroxyvalerate, collagen, albumin, gluten, chitosan, hyaluronate, cellulose, alignate, and starch.

In certain embodiments the biocompatible polymer comprises one or more cationic polymers. It is noted that such cationic polymers are particular well suited for delivery of a nucleic acid (e.g., DNA or mRNA). Illustrative cationic polymers include, but are not limited to poly-L-lysine (PLL), poly-ethylenimine (PEI; a branched cationic polymer, assisting in endosomal delivery), poly[(2-dimethylamino) ethyl methacrylate] (pDMAEMA), polyamidoamine (PAMAM) dendrimers, biodegradable poly(β-amino ester) (PBAE) polymers, poly(amino-co-ester) (PACE)-based polymers (branched amino acids, assisting in endosomal delivery), and the like (see, e.g., Wahane et al. (2020) Molecules, 25:2866).

In certain embodiments the biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), and poly(hydroxybutyrate-co-hydroxyvalerate). In certain embodiments the biocompatible polymer comprises poly(lactic-co-glycolic acid) (PLGA).

In certain embodiments the nanoparticle may be entirely composed of a biocompatible polymer (e.g., PLGA), while in other embodiments, the nanoparticle may comprise one or more lipids, including cationic lipids. We have achieved success with the method of Siewert et al. (2020) Cells, 9:2034), who demonstrated that the combination of two complexing ingredients, the cationic lipid, dioleoyl-3-trimethylammonium propane (DOTAP), and at the cationic polymer, protamine, could result in improved mRNA transfection in comparison to single complexing agents. In certain embodiments, the lipid(s) that can be included to bind to the nucleic acid, includes (but are not limited to) didodecyl-dimethylammonium bromide (DDAB), and 1,2-dioleoyloxy-3-trimethylammonium propane chloride (DOTAP). DOTAP is cationic lipid that can bind mRNA and nuclei acids, in addition to delivering danger signals. Other suitable lipids include DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino)propyl (12Z,15Z)-3-[(9Z,12Z)-octadeca-9,12-dien-1-yl]henicosa-12,15-dienoate (DMAP-BLP), (2Z)-non-2-en-1-yl 10-[(Z)-(1-methylpiperidin-4 yl) carbonyloxy]nonadecanoate (L101), and the like. In certain embodiments, inclusion of lipid can also be used to fine-tune the particle size and/or charge.

In certain embodiments the lipid comprises up to 25%, [molar percentage (excluding viral proteins or fragment(s) thereof)] of the nanoparticle. In certain embodiments the lipid comprises about 1%, or about 2%, or about 3%, or about 4%, or about 5%, or about 6%, or about 7% up to about 25%, or up to about 20%, or up to about 15%, or up to about 10% (molar percentage (excluding viral proteins or fragment(s) thereof)) of the nanoparticle.

Methods of making polymeric nanoparticles are well known to those of skill in the art (see, e.g., Marin et al. (2013) Int. J. Nanomed., 8:3071-3091, and references therein).

In certain embodiments the antigen(s) are encapsulated within the nanoparticle, and this can readily be accomplished by combining the antigen(s) with the biocompatible polymer during nanoparticle synthesis. In certain embodiments the antigen(s) are covalently attached to the surface of the nanoparticle, e.g., by adsorption or by coupling directly or using a linker.

Peptides/Proteins for Incorporation into Tolerogenic Nanoparticles.

In various embodiments the tolerogenic nanoparticles described herein comprise one or more peptide epitopes. In certain embodiments the nanoparticle comprise a single peptide epitope, while in other embodiments, the nanoparticles comprise a plurality of peptide epitopes. Where the nanoparticles comprise a plurality of peptide epitopes, the epitopes may each comprise a separate peptide or the epitopes may be linked together as a fusion protein.

Where multiple epitopes are provided in a single peptide, it is desirable to link the epitopes using a flexible peptide linker. In certain embodiments the need to provide flexible linkers can readily be met by using one of the most frequently used linker sequences, such as a stretch of Gly linked to Ser residues (“GS” linker). One example is the core sequence with the formula (Gly-Gly-Gly-Gly-Ser), (SEQ ID NO:57). In this example, the value of n can range from 1 up to 15. In one illustrative embodiment, the use of n=3 will allow the insertion of three GGGGS (SEQ ID NO:5 [[8]]) repeats between the individual epitopes cDNA sequencs. When these linkers are reversed translated into the cDNA sequence, it reads: ggcggcggcggcagcggcggcggcggcagcggcggcggcggcagc (SEQ ID NO: 7).

To allow epitope presentation, it is desirable for the epitopes to be proteolytically cleaved for insertion into the MHC-II complexmaking its way to the surface of LSECs. LSECs express the proprotein convertase, furin, in their trans-golgi network secretory pathway (Evdokimov et al. (2013) Biochem. Biophys. Res. Comm. 434:22-27). It has been shown that the protein, LEDA-1/PIANP, is proteolytically cleaved at aa 75-79 in humans and aa 69-73 in murine LSECs, allowing the cleaved fragments to be presented on the surface membrane. Additional furin substrates include blood clotting factors, serum proteins and growth factor receptors such as the insulin-like growth factor receptor. Furin prefers an Arg-X-Lys/Arg-Arg (SEQ ID NO:10) consensus sequence. The use of valine as amino acid, X, provides the sequence, RVRR (SEQ ID NO:911), which was inserted into our cDNA sequenceafter reverse translation to: cgcgtgcgccgc (SEQ ID NO:1012). This sequence was then inserted into the linker to yield the sequence shown in Table 1.

TABLE 1
Illustrative, but non-limiting
linker comprising a furin cleavage site.

| | SEQ |
|---|---|
| | ID |
| Linker | NO |
| ggc ggc ggc ggc agc cgc gtg cgc cgc ggc ggc ggc ggc agc | 11 |
| G G G G S R V R R ↓ G G G G S | 12 |
| Furin cleavage site↑ | |

In addition to furin, the cysteine proteases, cathepsin L (CL) and cathepsin S (CS), are known to play active roles in proteolytic cleavage in the MHC-II endosomal compartment. Antigen cleavage by these proteases releases peptides, capable of completing with the degraded invariant (li) chain for MHC class II epitope presentation (see, e.g., Hsieh et al. (2002) J. Immunol. 168:2618-2625). Cathepsin S recognizes the peptide sequence PMGLP (SEQ ID NO:13), which is cleaved between glycine and lysine. Reverse translation of the cleavage site yields a ccgatgggcctgccg (SEQ ID NO:15) sequence. Cathepsin L recognizes the peptide sequence HKFR (SEQ ID NO:16), which upon reverse translation yield the following sequence: cataaatttcgc (SEQ ID NO:17). It would be straightforward to exchange the currently designed furin cleavage site with either of the cathepsin recognition sites.

Endogenous CD4 epitopes can be directed to MHC-II delivering endosomes from the cytosol by using the TfR1-118 targeting domain (Diebold et al. (2001) Gene Therapy, 8:487-493). We designed a novel fusion protein construct that attaches the nucleotide sequence for TfR1-118 upstream of the stringed-together nucleotide sequences, encoding for various epitopes. The membrane-anchoring region of the transferrin receptor (amino acids 1-118), allows the fusion protein construct to be transported to the LSEC surface membrane. From here, the expressed fusion protein can be endocytosed into the MHC class II compartment, where proteolytic cleavage will release the epitopes for insertion into the peptide groove of MHC-II complex. An alternative targeting approach is to use the invariant li chain for direct peptide transport into the MHC-II endosomal compartment. This allows direct targeting of li/Ara h peptide fusions to the MHC II processing pathway.

Nucleic Acids for Incorporation into Tolerogenic Nanoparticles.

In various embodiments the tolerogenic nanoparticles comprise one or more nucleic acids that can encode one or more epitopes described herein.

In certain embodiments the immunogenic nanoparticles described herein comprise one or more nucleic acids that encode proteins comprising one or more antigen(s)/epitopes to which a tolerogenic immune response is to be induced. In certain embodiments the nucleic acid comprises a DNA. In certain embodiments the nucleic acid comprises an mRNA.

The core principle behind mRNA is to deliver nucleic acid constructs, encoding for one or more epitope(s), to the host cell cytoplasm where translation generates protein or peptide sequences, which are localized intracellularly, secreted or translocated to the surface membrane.

In certain embodiments the mRNA comprises one of two categories of mRNA constructs: 1) Non-replicating mRNA (NRM); and 2) Self-amplifying mRNA (SAM) constructs. NRM and SAM constructs typically comprise a cap structure, 5′ and 3′ untranslated regions (UTRs), an open-reading frame (ORF) endcoding the antigen(s) of interest, and a 3′ poly(A) tail (see, e.g., Pardi (2018) Nat. Rev. Drug Dis. 17:261-279). SAMs differ from NRM consturcts by additionally including genetic replication machinery derived from positive-stranded mRNA viruses, most commonly from alphaviruses such as Sindbis and Semliki-Forest viruses (see, e.g., Cheng et al. (2001) J. Virol. 75:2368-2376; Ljungberg & Liljestrom (2015) Exp. Rev. Vacc. 14:177-194; and the like). Generally, the ORF encoding viral structural proteins is replaced by the selected transcript of interest, and the viral RNA-dependent RNA polymerase is retained to direct cytoplasmic amplification of the replicon construct.

Once delivered to the cytosol, mRNA (or DNA) constructs are promptly translated by ribosomes to produce the protein of interest (e.g., comprising the antigen(s)/epitope(s)). Once released, these translation products undergo post-translational modification such as protein folding or phosphorylation. Similarly, SAM constructs can also be immediately translated by ribosomes to produce the replicase machinery necessary for self-amplification of the mRNA. Self-amplified mRNA constructs are translated by ribosomes to produce the protein of interest, which may undergo post-translational modification. The innate and adaptive arms of the immune responsefurther process the expressed antigen to generate an appropriate response. T.

In various embodiments, mRNA vaccine manufacturing begins with the generation of a plasmid DNA (pDNA) containing a DNA-dependent RNA polymerase promoter, such as T7 (see, e.g., Rong et al. (1998) Proc. Natl. Acad. Sci. USA, 95:515-519) and the corresponding sequence for the mRNA construct. The pDNA is linearized to serve as a template for the DNA-dependent RNA polymerase to transcribe the mRNA, and subsequently degraded by a DNase process step. In various illustrative, but non-limiting embodiments the addition of the 5′ cap and the 3′ poly(A) tail can be achieved during the in vitro transcription step (see, e.g., Stepinski et al. (2001) RNA, 7:1486-1495; Grudzien-Nogalska et al. (2007) Meth. Enzym. Ch. 431:203-227; and the like) or enzymatically after transcription (see, e.g., Martin & Moss (1975) J. Bio Chem. 250:9330-9335). Enzymatic addition of the cap can be accomplished by using, for example, guanylyl transferase and 2′-O-methyltransferase to yield a Cap 0 (^{N7Me}GpppN) or Cap 1 (^{N7Me}GpppN^{2′OMe}) structure, respectively, while the poly-A tail can be provided through enzymatic addition via poly-A polymerase.

In various embodiments purification or the mRNA construct can be achieved with the application of high-pressure liquid chromatography (HPLC). The resultant drug substance is then formulated into drug nanoparticle delivery system described herein.

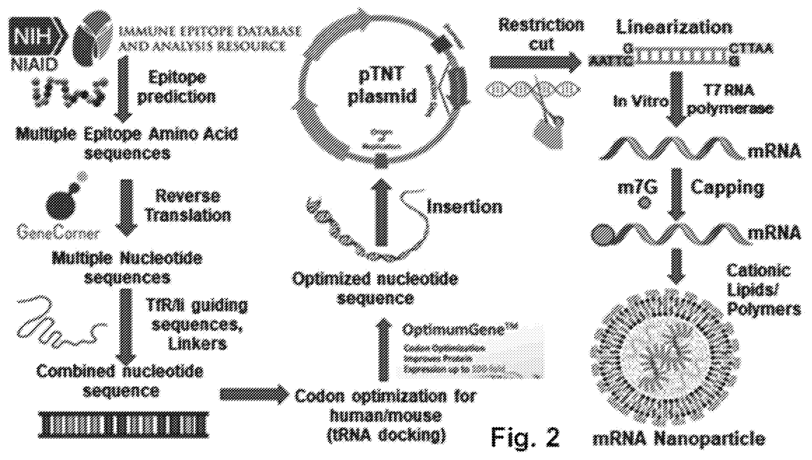

One illustrative scheme for preparing the nucleic acid delivering tolerogenic nanoparticles described herein is shown in FIG. 2. As illustrated therein, a first step is reverse translation of the peptide into cDNA nucleotide sequences. Where multiple epitopes are encoded by a single nucleic acid, in certain embodiments, the nucleic acid encodes linker sequences (e.g., as described above) for spacing and release of the individual peptide epitopes after peptide cleavage.

In addition, in various embodiments, the cDNA construct also encodes targeting sequences that allow antigen access to the MHC-II endosomal compartment for peptide presentation to Treg precursors. Protein subdomains of the transferrin receptor (TfR) or the invariant chain (Ii) (which controls peptide loading into the MHC-II complex) have been shown to be effective targeting sequences for immunogenic ovalbumin epitopes (see, e.g., Diebold et al. (2001) Gene Therapy, 8:487-493). The transferrin receptor is a membrane glycoprotein that mediates cellular iron uptake from the surface membrane after transferrin binding. Research has shown that a fusion protein constructed from the membrane anchoring domain of this receptor (amino acids 1-118) plus a MHC-II binding epitope (e.g., aa 323-339) from ovalbumin allows MHC-II access and epitope expression by APC's (Id). The invariant (li) chain is a polypeptide that plays a role in epitope loading by MHC-II complexes. This peptide has MHC II access and can be used to make hybrid constructs that can enter the MHC-II compartment from the cytosol for presentation to CD4⁺ T-cells. Thus, li-OVA fusion has been shown to directly target the MHC class II processing pathway (Id). Given this background, in certain embodiments the nucleotide sequence for the TfR 1-118 domain will be added to the cDNA construct upstream of the epitope encoding nucleotide sequences. However, the same outcome can be accomplished by using the nucleotide sequence for the li-chain (114 aa).

The nanoparticles are formulated so that the mRNA construct once released into the cytoplasm of a cell, effectively utilizes the translational machinery of the host cell to generate a sufficient quantity of the encoded epitope(s) for antigen presentation by MHC-II. In this regard, a number of parameters can be exploited to optimize the designed sequence of the particular nucleic acid construct. These include, but are not limited to amount/concentration of the nucleic acid in the delivery particle, 5′ capping efficiency; UTR structure, length, and regulatory elements; modification of coding sequence; and poly-A-tail properties.

Thus, for example, the length of the 3′ UTR, 5′ UTR structures, and regulatory elements in both UTRs can impact transcription efficiency. The 5′ 7-methylguanosine (m7G) cap of the mRNA molecule, linked via a triphosphate bridge to the first transcribed nucleotide, facilitates efficient translation, and blocks 5′-3′ exonuclease-mediated degradation. The specific cap structure can play an important role in both protein production and immunogenicity, with incomplete capping (5′ triphosphate) and Cap 0 structures shown to stimulate RIG-1 (see, e.g., Devarkar et al. (2016) Proc. Natl. Acad. Sci. USA, 113:596-601; Xu et al. (2018) Protein Cell, 9:246-253; Lassig & Hopfner, (2017) J. Biol. Chem. 292:9000-9009; and the like). Additionally, 2-O′-unmethylated capped RNA can be sequestered by cellular IFN-induced proteins with tetratricopeptide repeats (IFIT1) that prevent the initiation of translation or detected by the cytoplasmic RNA sensor MDA5. Typically, the choice of enzyme and reaction conditions are optimized in order to catalyze the highest percentage of cap formation. Additionally, the poly(A) tail and its properties such as length, can be optimized for translation and protection of the mRNA molecule (see, e.g., Goldstrohm & Wickens (2008) Nat. Rev. Mol. Cell Biol. 9:337-344; Azoubel Lima et al. (2017) Nat. Struct. Mol. Biol. 24:1057-1064; and the like)).

In certain embodiments codon optimization and modification of nucleotides can contribute to translation efficiency. For example, optimization of guanine and cytosine (GC) content can have a significant impact (see, e.g., Kudla et al. (2016) Plos Biol. 4: e180). The innate immune activation to mRNA can also influence its utility as a delivery system. The use of modified nucleosides, such as pseudouridine or N-1-methylpseudouridine to remove intracellular signaling triggers for protein kinase R (PKR) activation, can provide enhanced antigen expression and adaptive immune responses (see, e.g., Anderson et. al.. (2010) Nucleic Acids Res. 38:5884-5892; Andries et al. (2015) J. Contr. Rel. 217:337-344; Pardi et al. (2018) J. Exp. Med. 215:1571-1588; and the like).

In view of these considerations, in certain embodiments, codon optimization is used for the integrated cDNA construct to allow optimal gene expression in accordance with the tRNA pool of human versus non-human cells. The optimized cDNA sequence is then inserted into a bacterial plasmid vector, such as pTNT (Promega). This vector contains tandem T7 promoters for transcription of mRNA, a 5′ UTR from rabbit β-globin, and a synthetic poly(A) 30 tail, important features for efficient protein translation. Other plasmid vectors with similar functional components are also available for our use.

Following plasmid amplification and purification, we linearize our plasmids by restriction enzyme cutting, to facilitate in vitro transcription into mRNA constructs through the use of T7 polymerase. A 7-methyl-guanosine cap is added to the 5′ end of RNA using a vaccinia virus capping enzyme. The cap is essential for mRNA stability and efficient translation. The mRNA coding sequence is further modified by a non-natural RNA nucleobase N1-methylpseudouridine (m14′) to reduce the likelihood that the nucleic acid construct will trigger robust immunological danger signals (e.g., through interaction with toll-like receptors). We want to avoid a strong adjuvant effects by the RNA strand, which can interfere with Treg generation. Following the construction of the integrated mRNA strand, the nucleic acid construct will be encapsulated in polymer and lipid nanoparticles to prevent RNA degradation during in vivo delivery to APC in the liver or elsewhere. These nanoparticle constructs will be discussed in a later section.

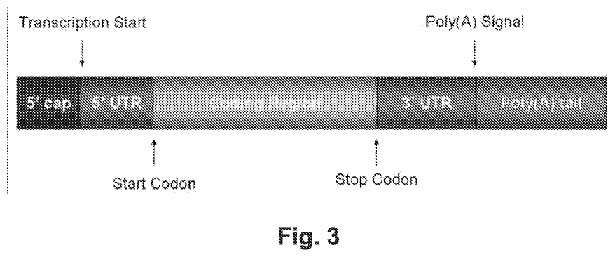

To round off the design of the cDNA coding sequence, we add start codon and stop codons, as shown in FIG. 3. The untranslated region, 5′ UTR and 3′ UTR region are pre-selected sequences inserted into commercially available plasmids (e.g., pTNT from Promega, CleanCap plasmid from TriLink) for mRNA preparation. The 5′ cap and poly(A) tail is added post transcriptionally (FIG. 3).

It will be appreciated that the foreign nucleic acid constructs and manufacturing methods are illustrative and non-limiting and using the teaching provided herein, numerous other constructs and manufacturing methods will be available to one of skill in the art.

First (LSEC-Binding), and/or Second, and/or Third Targeting Moieties.

In various embodiments the tolerogenic nanoparticles described herein comprise a targeting moiety (e.g., a first targeting moiety that binds to a liver sinusoidal endothelial cell (LSEC). In certain embodiments such a targeting moiety binds to a scavenger receptor in the liver (and/or on macrophages in the liver).

Scavenger receptors are receptors on macrophages and other cells that bind to numerous ligands, such as bacterial cell-wall components, and remove them from the blood. The Kupffer and liver sinusoidal endothelial cells are particularly rich in scavenger receptors. Illustrative scavenger receptors in the liver and/or on macrophages in the liver include, but are not limited to stabilin-1, stabilin-2, SCARA1 or MSR1, SCARA2 or MARCO, SCARA3, SCARA4 or COLEC12, SCARA5, SCARB1, SCARB2, SCARB3 or CD36, and the like. In certain embodiments the first targeting moiety comprise a moiety (e.g., a peptide) that binds to stabilin-1 and/or to stabilin-2.

Ligands that bind to scavenger receptors are well known to those of skill in the art and can readily be incorporated as a first targeting moiety in the tolerogenic nanoparticles described herein. In certain embodiments, for example, a suitable ligand that binds to stabilin-1 and/or to stabilin-2 comprises a fragment of the apoB protein. In certain embodiments, the fragment ranges in length from about 5 up to about 50, or up to about 40, or up to about 30, or up to about 20 amino acids. In certain embodiments the fragment ranges in length from about 5 up to about 20, or up to about 10 amino acids. In certain embodiments the first targeting moiety is a peptide comprising the amino acid sequence RKRGLK (SEQ ID NO: 18). In certain embodiments the first targeting moiety is a peptide comprising the amino acid sequence RLYRKRGLK (SEQ ID NO:19). In certain embodiments the first targeting moiety is a peptide comprising the amino acid sequence CGGKLGRKRYLR (SEQ ID NO: 4). In certain embodiments the surface can be coated by sugars such as mannan as well as targeting moieties such as aptamers and the like. Aptamers are oligonucleotide molecules that exhibit 3D structure to allow them to bind to specific target molecules. Aptamers are usually created by selecting them from a random sequence pool but natural aptamers also exist.

In certain embodiments the tolerogenic nanoparticle can optionally comprise a a second targeting moiety that binds to a mannose receptor in the liver, and/or a third targeting moiety that binds to hepatocytes.

The mannose receptor (cluster of differentiation 206, CD206) is a C-type lectin primarily present on the surface of macrophages, immature dendritic cells and liver sinusoidal endothelial cells. Ligands that bind to the mannose receptor are well known to those of skill in the art and can readily be incorporated as a second targeting moiety into the tolerogenic nanoparticles described herein. Illustrative, but non-limiting examples of ligands for the mannose receptor include, but are not limited to mannan, mannose, N-acetyl-glucosamine, and fucose. In certain embodiments the second targeting moiety comprises a mannan (e.g., a mannan ranging from about 35 to about 30 kDa) that binds to LSECs.

Ligands that bind to hepatocytes are well known to those of skill in the art and can readily be incorporated as a third targeting moiety targeting moiety in the tolerogenic nanoparticles described herein. Thus, for example, asialoorosomucoid, galactoside, a galactosamine (e.g., GalNAC), asialofetuin, sterylglucoside, lactose/lactobionic acid, PVLA (poly-(N-p-vinylbenzyl-O-beta-D-galactopyranosyl-[1-4]-D-gluconamide) target asialoglycoprotein receptors on hepatocytes. Apolipoprotein A-I is a ligand for scavenger receptor class B type I receptors present on hepatocytes. Linoleic acid targets plasma membrane fatty acid binding protein hepatocytes, while glycyrrhizin receptors are targeted by glycyrrhizin and heparan sulphate receptors by acetyl-CKNEKKNKIERNNKLKQPP-amide (SEQ ID NO:20) ligand; both receptors present on hepatocytes.

The foregoing targeting moieties are illustrative and non-limiting. Using the teachings provided herein, other targeting moieties for a scavenger receptor in the liver (and/or on macrophages in the liver) or for a mannose receptor in the liver, or for binding to hepatocytes will be available to one of skill in the art and readily incorporated into the nanoparticles described herein.

In certain embodiments the first targeting moiety and/or the second targeting moiety, and/or third targeting moiety can be attached to the nanoparticle by simple adsorption or by non-covalent linkage. Useful non-covalent linkages include, but are not limited to, affinity binding pairs, such as biotin-streptavidin and immunoaffinity, having sufficiently high affinity to maintain the linkage during use. Such non-covalent linkers/linkages are well known to those of skill in the art.

In certain embodiments the first targeting moiety and/or the second targeting moiety, and/or the third targeting moiety can be covalently coupled to the nanoparticle directly or through a linker. The art is also replete with conjugation chemistries useful for covalently linking a targeting moiety to a second moiety (e.g., a biocompatible polymer). Art-recognized covalent coupling techniques are disclosed, for instance, in U.S. Pat. Nos. 5,416,016, 6,335,435, 6,528,631, 6,861,514, 6,919,439, and the like. Other conjugation chemistries are disclosed in U.S. Patent Publication No. 2004/0249178. Still, other conjugation chemistries include: p-hydroxy-benzoic acid linkers (see, e.g., Chang-Po et al. (2002) Bioconjugate Chem. 13 (3): 525-529), native ligation (see, e.g., Stetsenko et al. (2000) J. Org. Chem. 65:4900-4908), disulfide bridge conjugates (see, e.g., Oehlke et al. (2002) Eur. J. Biochem. 269:4025-4032; Rogers et al. (2004) Nucl. Acids Res. 32 (22): 6595-6604), maleimide linkers (see, e.g. Zhu et al. (1993) Antisense Res Dev. 3:265-275), thioester linkers (see, e.g., Ede et al. (1994) Bioconjug. Chem. 5:373-378), Diels-Alder cycloaddition (see, e.g., Marchan et al. (2006) Nucl. Acids Res. 34 (3): e24); U.S. Pat. No. 6,656,730 and the like). For reviews of conjugation chemistries, see also Tung et al. (2000) Bioconjugate Chem. 11:605-618, Zatsepin et al. (2005) Curr. Pharm. Des. 11 (28): 3639-3654, Juliano (2005) Curr. Opin. Mol. Ther. 7 (2): 132-136, and the like. While certain of the foregoing chemistries are utilized for nucleic acid-peptide conjugation one of skill will recognize that they can readily be modified for attachment of first and/or second targeting moieties to the nanoparticles.

In one illustrative, but non-limiting embodiment the first (LSEC binding) targeting moiety is attached to the nanoparticle using a linker (e.g., the NAEM maleimide) while the second targeting moiety, when present, is attached to the nanoparticle by adsorption, or by direct covalent conjugation (e.g., via conjugation of the hydroxyl-terminus of a peptide targeting moiety to a COOH terminal group on the nanoparticle polymer).

It is also noted that the LPN nanoparticles can also directly target CD4 T cells through lipid modification and/or attachment of an antibody. Thus, it is also possible that in addition to enhancement of antigen presentation to naïve T cells, for development into Tregs, it is possible to also functionally impact the Tregs themselves, including putting into these particles a series of lipophilic Treg promoters such as mTOR inhibitors, agonists for peroxisome proliferator-activator receptors (PPARa, b, g). liver X receptors (LXRa, b), lipoxins, RAR agonists (active form of vitamin A, all-trans retinoic acid).

The foregoing targeting moieties and methods of attachment to the nanoparticle(s) are illustrative and non-limiting. Using the teaching provided herein numerous other targeting moieties and attachment chemistries will be available to one of skill in the art.

### Antigens/Epitopes for Inclusion in Tolerogenic Nanoparticles.

In various embodiments the antigen(s) comprising (or encoded by nucleic acids) the tolerogenic nanoparticles can include any moiety for which it is desirable to induce tolerance and/or to reduce immunogenicity. It will be recognized that in certain embodiments the tolerogenic nanoparticles comprise (or encode) a single antigen and/or epitope, while in other embodiments the tolerogenic nanoparticles comprise (or encode) 2, 3, 4, 5, 6, 7, 8, 9, or 10, or more antigen(s) and/or epitopes. In certain embodiments, where the nanoparticles comprise or encode two or more antigens, the antigens can be provided as separate molecules/moieties or the nucleic acids encoding the antigen(s) can be can be provided as separate molecules/moieties. In certain embodiments the antigens can be provided so that they are presented as separate epitopes on a single molecule/moiety (e.g., as separate epitopes on a single protein). In certain embodiments the antigen(s), or nucleic acid(s) encoding the antigen(s), are disposed within the nanoparticle, while in other embodiments one or more of the antigen(s) or nucleic acids encoding the antigens can be provided attached to the surface of the nanoparticle.

Illustrative antigens include but are not limited to a material selected from the group consisting of a peptide, a nucleic acid, a glycoprotein, sugar, a nucleoprotein and a carbohydrate. In certain embodiments the antigen(s) comprise one or more peptides or nucleic acids encoding one or more antigenic/tolerogenic peptide epitopes. In certain embodiments the peptides epitope(s) range in length from about 5 amino acids, or from about 8 amino acids, or from about 10 amino acids, or from about 15 amino acids up to about 60 amino acids, or up to about 50 amino acids, or up to about 40 amino acids, or up to about 30 amino acids. In certain embodiments the peptide can be posttranslationally modified to constitute neoepitopes, e.g., phosphorylation, citrullination, deamidation, oxidation, carbonylation, peptide fusion or alternative splicing (James et al. Diabetes Volume 67, June 2018).

In various embodiments the tolerogenic nanoparticles are designed and synthesized to induce tolerance to various allergies (e.g., peanut allergy, house dust mite allergies), to suppress the production of anti-drug antibodies, to treat and/or prevent diabetes, to treat and/or prevent lupus, and/or to prevent or suppress antibodies directed against viral vectors (e.g., AAV).

### Peanut Allergy.

In certain embodiments the tolerogenic nanoparticles are designed for the treatment and/or prophylaxis of an allergic disease such as, for example, a peanut allergy or a house dust mite allergy. The development of epitope peptide therapy to treat allergic disorders in humans have accumulated clinical trial evidence indicating that immune-dominant T-cell epitopes can be used effectively for desensitization against cat, house dust mite and other allergens. However, the immunological mechanism(s) that underpin successful therapy outcome still needs to be clarified. While there is currently no peptide-based immunotherapy in the marketplace, epitope mapping for a number of allergens have identified critical amino acid sequences that can be presented to T-cells by Type II HLA alleles, without the danger of triggering mast cell release through IgE crosslinking.

Food allergy treatment with peptide-based therapeutics is attracting significant attention. This includes consideration of the use of tolerogenic T-cell epitopes for the treatment of peanut anaphylaxis, provided that:
The selection of dominant CD4 T-cell epitopes for major peanut allergens consider the need for promiscuous MHC-II binding, thereby allowing epitope presentation by a wide array of HLA alleles in peanut allergic populations;
Peptide selection to avoid IgE binding fragments that can interact and cross-link cell-bound IgE;
Platform scalability and biocompatibility, allowing upscale production and treatment safety; and
The treatment can induce durable immune suppression, achievable by natural tolerogenic antigen presenting cells such as LSECs.

As illustrated herein in Example 3, the animal data presented show the feasibility of successful immunotherapy, and support the contention that T-cell epitopes can be selected in humans to serve as lead tolerogenic peptide sequences for therapeutic use. In order to identify lead candidate sequences, our first task was to comprehensively retrieve a pool of Ara-h1, -h2, -h3 and -h6 sequences from the Immune Epitope and Database Resource (IEDB) and the literature. Ara-h1, -h2, -h3 and -h6 are the major peanut allergens shown to play a role in anaphylactic responses in humans. This information for was further refined for using the design principles demonstrated in our preclinical studies.

The IEDB predictions for murine Ara h2 and Ara h6 T-cell epitopes (Table 66 in Example 3) took into consideration the IEDB database and resource (//www.iedb.org/result_v3.php?cookie_id=a2c230) to make predictions for Ara h epitopes, including affinity binding to murine MHC-II sequences (e.g., I^{E}) for animal experimentation.

In contrast to mice, the binding interactions for T-cell epitope candidates in humans must take into consideration data for HLA class II molecules encoded by one of three highly polymorphic loci, HLA-DR, HLA-DP, or HLA-DQ. Search of the IEDB for a potential list of human T-cell epitope sequences for the major peanut allergens allowed us to construct Tables 2-5 below. These peptide sequences are available for further testing and refinement to derive a condensed list of human peptide sequences that can used for tolerogenic immunotherapy. An important prediction tool is the use of human peripheral blood mononuclear cells (PBMC) to study cellular proliferation and cytokine production (e.g., IFN-γ, IL-5, IL-4, IL-13), involving techniques such as CFSE labeling, ELISA, Elispot assays or tetramer staining. This selection process also allowed the use of published studies to further evaluate the IEDB sequences, as discussed herein. Further refinement involved the assessment of peptide characteristics important in nanoparticles encapsulation, promiscuous binding interactions with diverse HLA alleles and screening predictions that demonstrate the ability to generate regulatory T cells. The following information was retrieved for Ara-h1, -h2, -h3 from the IEDB (Tables 2-5) either as deposited sequences or making database predictions for possible Ara h6 epitopes and.

TABLE 2
Experimentally deposited and tested linear
Ara h 1 epitopes in the IEDB for humans.

| | | Starting | Ending |
|---|---|---|---|
| Epitope IRI | Sequence | Position | Position |
| www.iedb.org/epitope/98735 | DNILVIQQGQATVTV | 233 | 247 |
| | (SEQ ID NO: 21) | | |
| www.iedb.org/epitope/98785 | GALMLPHFNSKAMVI | 441 | 455 |
| | (SEQ ID NO: 22) | | |
| www.iedb.org/epitope/98841 | KAMVIVVVNKGTGNLELVAV | 451 | 470 |
| | (SEQ ID NO: 23) | | |
| www.iedb.org/epitope/98866 | LEAAFNAEFNEIRRV | 321 | 335 |
| | (SEQ ID NO: 24) | | |
| www.iedb.org/epitope/98910 | NEGVIVKVSKEHVEE | 361 | 375 |
| | (SEQ ID NO: 25) | | |
| www.iedb.org/epitope/98963 | QLQDLDMMLTCVEIK | 425 | 439 |
| | (SEQ ID NO: 26) | | |
| www.iedb.org/epitope/98971 | QRSRQFQNLQNHRIV | 201 | 215 |
| | (SEQ ID NO: 27) | | |
| www.iedb.org/epitope/98975 | QVEKLIKNQKESHFV | 569 | 583 |
| | (SEQ ID NO: 28) | | |
| www.iedb.org/epitope/99213 | ELVAVRKEQQQRGRR | 466 | 480 |
| | (SEQ ID NO: 29) | | |
| www.iedb.org/epitope/99233 | EVRRYTARLKEGDVF | 496 | 510 |
| | (SEQ ID NO: 30) | | |
| www.iedb.org/epitope/99355 | ISYILNRHDNQNLRV | 271 | 285 |
| | (SEQ ID NO: 31) | | |
| www.iedb.org/epitope/99364 | KAMVIVVVNKGTGNL | 451 | 465 |
| | (SEQ ID NO: 32) | | |
| www.iedb.org/epitope/99442 | LQRFDQRSRQFQNLQ | 196 | 210 |
| | (SEQ ID NO: 33) | | |
| www.iedb.org/epitope/99511 | NQNLRVAKISMPVNT | 280 | 294 |
| | (SEQ ID NO: 34) | | |
| www.iedb.org/epitope/99599 | RCTKLEYDPRCVYDP | 61 | 75 |
| | (SEQ ID NO: 35) | | |
| www.iedb.org/epitope/99616 | RKSFNLDEGHALRIP | 253 | 267 |
| | (SEQ ID NO: 36) | | |
| www.iedb.org/epitope/99676 | SNREVRRYTARLKEG | 493 | 507 |
| | (SEQ ID NO: 37) | | |
| www.iedb.org/epitope/99688 | SSELHLLGFGINAEN | 523 | 537 |
| | (SEQ ID NO: 38) | | |
| www.iedb.org/epitope/99762 | VLASVSATHAKSSPY | 16 | 30 |
| | (SEQ ID NO: 39) | | |
| www.iedb.org/epitope/100137 | IFLAGDKDNVIDQIEKQAKD | 541 | 560 |
| | (SEQ ID NO: 40) | | |
| www.iedb.org/epitope/100169 | ISYILNRHDNQNLRVAKISM | 271 | 290 |
| | (SEQ ID NO: 41) | | |
| www.iedb.org/epitope/100251 | LEAAFNAEFNEIRRVLLEEN | 321 | 340 |
| | (SEQ ID NO: 42) | | |
| www.iedb.org/epitope/100312 | MRGRVSPLMLLLGILVLASV | 1 | 20 |
| | (SEQ ID NO: 43) | | |
| www.iedb.org/epitope/100323 | NEGVIVKVSKEHVEELTKHA | 361 | 380 |
| | (SEQ ID NO: 44) | | |
| www.iedb.org/epitope/100445 | QRSRQFQNLQNHRIVQIEAK | 201 | 220 |
| | (SEQ ID NO: 45) | | |
| www.iedb.org/epitope/100462 | RFSTRYGNQNGRIRVLQRFD | 181 | 200 |
| | (SEQ ID NO: 46) | | |
| www.iedb.org/epitope/100488 | RTRGRQPGDYDDDRRQPRRE | 91 | 110 |
| | (SEQ ID NO: 47) | | |
| www.iedb.org/epitope/148572 | DNILVIQQGQATVTVANGNN | 233 | 252 |
| | (SEQ ID NO: 48) | | |
| www.iedb.org/epitope/148579 | EEEGSNREVRRYTARLKEGD | 489 | 508 |
| | (SEQ ID NO: 49) | | |
| www.iedb.org/epitope/148597 | FNEIRRVLLEENAGGEQEER | 329 | 348 |
| | (SEQ ID NO: 50) | | |
| www.iedb.org/epitope/148649 | KEGDVFIMPAAHPVAINASS | 505 | 524 |
| | (SEQ ID NO: 51) | | |
| www.iedb.org/epitope/148658 | LELVAVRKEQQQRGRREEEE | 465 | 484 |
| | (SEQ ID NO: 52) | | |
| www.iedb.org/epitope/148671 | LQNHRIVQIEAKPNTLVLPK | 209 | 228 |
| | (SEQ ID NO: 53) | | |
| www.iedb.org/epitope/148695 | NNFGKLFEVKPDKKNPQLQD | 409 | 428 |
| | (SEQ ID NO: 54) | | |
| www.iedb.org/epitope/148699 | NSKAMVIVVVNKGTGNLELV | 449 | 468 |
| | (SEQ ID NO: 55) | | |
| www.iedb.org/epitope/148770 | QKESHFVSARPQSQSQSPSS | 577 | 596 |
| | (SEQ ID NO: 56) | | |
| www.iedb.org/epitope/148933 | SKEHVEELTKHAKSVSKKGS | 369 | 388 |
| | (SEQ ID NO: 57) | | |
| www.iedb.org/epitope/148966 | TSRNNPFYFPSRRFSTRYGN | 169 | 188 |
| | (SEQ ID NO: 58) | | |
| www.iedb.org/epitope/148985 | VVNKGTGNLELVAVRKEQQQ | 457 | 476 |
| | (SEQ ID NO: 59) | | |
| www.iedb.org/epitope/190700 | AAHPVAINASSELHLLGFGI | 514 | 533 |
| | (SEQ ID NO: 60) | | |
| www.iedb.org/epitope/190702 | AGPREREREEDWRQPREDWR | 118 | 137 |
| | (SEQ ID NO: 61) | | |
| www.iedb.org/epitope/190706 | ALMLPHFNSKAMVIVVV | 442 | 458 |
| | (SEQ ID NO: 62) | | |
| www.iedb.org/epitope/190707 | ALMLPHFNSKAMVIVVVNKG | 442 | 461 |
| | (SEQ ID NO: 63) | | |
| www.iedb.org/epitope/190708 | AMVIVVVNKGTGNLELVAV | 452 | 470 |
| | (SEQ ID NO: 64) | | |
| www.iedb.org/epitope/190716 | AVRKEQQQRGRREEEEDEDE | 469 | 488 |
| | (SEQ ID NO: 65) | | |
| www.iedb.org/epitope/190719 | CQQEPDDLKQKACESRCTKL | 46 | 65 |
| | (SEQ ID NO: 66) | | |
| www.iedb.org/epitope/190724 | DEEEEGSNREVRRYTARLKE | 487 | 506 |
| | (SEQ ID NO: 67) | | |
| www.iedb.org/epitope/190729 | DLSNNFGKLFEVKPDKKNPQ | 406 | 425 |
| | (SEQ ID NO: 68) | | |
| www.iedb.org/epitope/190734 | EDWRQPREDWRRPSHQQPRK | 127 | 146 |
| | (SEQ ID NO: 69) | | |
| www.iedb.org/epitope/190741 | EQVEKLIKNQKESHFVSARP | 568 | 587 |
| | (SEQ ID NO: 70) | | |
| www.iedb.org/epitope/190745 | EVKPDKKNPQLQ | 413 | 424 |
| | (SEQ ID NO: 71) | | |
| www.iedb.org/epitope/190746 | EVRRYTARLKEGDVFIMPAA | 496 | 515 |
| | (SEQ ID NO: 72) | | |
| www.iedb.org/epitope/190748 | FDQRSRQFQNLQNHRIVQIE | 199 | 218 |
| | (SEQ ID NO: 73) | | |
| www.iedb.org/epitope/190749 | FEDFFPASSRDQSSYLQGFS | 295 | 314 |
| | (SEQ ID NO: 74) | | |
| www.iedb.org/epitope/190750 | FEVKPDKKNPQLQDLDMMLT | 415 | 434 |
| | (SEQ ID NO: 75) | | |
| www.iedb.org/epitope/190753 | FNAEFNEIRRVLLEENAGGE | 322 | 341 |
| | (SEQ ID NO: 76) | | |
| www.iedb.org/epitope/190757 | FQNLQNHRI | 206 | 214 |
| | (SEQ ID NO: 77) | | |
| www.iedb.org/epitope/190758 | FQNLQNHRIVQIEAKPNTLV | 206 | 225 |
| | (SEQ ID NO: 78) | | |
| www.iedb.org/epitope/190760 | FSRNTLEAAFNAEFNEIRRV | 313 | 332 |
| | (SEQ ID NO: 79) | | |
| www.iedb.org/epitope/190764 | GDVFIMPAAHPVAINASS | 507 | 524 |
| | (SEQ ID NO: 80) | | |
| www.iedb.org/epitope/190766 | GEQEERGQRRWSTRSSENNE | 340 | 359 |
| | (SEQ ID NO: 81) | | |
| www.iedb.org/epitope/190767 | GEQEWGTPGSHVREETSRNN | 154 | 173 |
| | (SEQ ID NO: 82) | | |
| www.iedb.org/epitope/190771 | GINAENNHRIFLAGDKDNVI | 532 | 551 |
| | (SEQ ID NO: 83) | | |
| www.iedb.org/epitope/190777 | GRREEEEDEDEEEEGSNREV | 478 | 497 |
| | (SEQ ID NO: 84) | | |
| www.iedb.org/epitope/190780 | HAKSVSKKGSEEEGDITNPI | 376 | 395 |
| | (SEQ ID NO: 85) | | |
| www.iedb.org/epitope/190781 | HALRIPSGFISYILNRHDNQ | 262 | 281 |
| | (SEQ ID NO: 86) | | |
| www.iedb.org/epitope/190791 | IEAKPNTLVLPKHADADNIL | 217 | 236 |
| | (SEQ ID NO: 87) | | |
| www.iedb.org/epitope/190796 | ILVIQQGQATVTVANGNNRK | 235 | 254 |
| | (SEQ ID NO: 88) | | |
| www.iedb.org/epitope/190802 | KAMVIVVVNKG | 451 | 461 |
| | (SEQ ID NO: 89) | | |
| www.iedb.org/epitope/190804 | KDLAFPGSGEQVEKLIKNQK | 559 | 578 |
| | (SEQ ID NO: 90) | | |
| www.iedb.org/epitope/190808 | KEHVEELTKHAKSVSKKGSE | 367 | 386 |
| | (SEQ ID NO: 91) | | |
| www.iedb.org/epitope/190811 | KGTGNLELVAVRKEQQQRGR | 457 | 476 |
| | (SEQ ID NO: 92) | | |
| www.iedb.org/epitope/190820 | KLEYDPRCVYDPRGHTGTTN | 64 | 83 |
| | (SEQ ID NO: 93) | | |
| www.iedb.org/epitope/190848 | LLLGILVLASVSATHAKSSP | 10 | 29 |
| | (SEQ ID NO: 94) | | |
| www.iedb.org/epitope/190849 | LPKHADADNILVIQQGQATV | 226 | 245 |
| | (SEQ ID NO: 95) | | |
| www.iedb.org/epitope/190854 | LTCVEIKEGALMLPHFNSKA | 433 | 452 |
| | (SEQ ID NO: 96) | | |
| www.iedb.org/epitope/190869 | NGRIRVLQRFDQRSRQFQNL | 190 | 209 |
| | (SEQ ID NO: 97) | | |
| www.iedb.org/epitope/190875 | NLQNHRIVQIEAKPNTLVLP | 208 | 227 |
| | (SEQ ID NO: 98) | | |
| www.iedb.org/epitope/190876 | NNFGKLFEVKPDKKNPQ | 409 | 425 |
| | (SEQ ID NO: 99) | | |
| www.iedb.org/epitope/190877 | NNFGKLFEVKPDKKNPQLQ | 409 | 427 |
| | (SEQ ID NO: 100) | | |
| www.iedb.org/epitope/190878 | NNPFYFPSRRFSTRYGNQNG | 172 | 191 |
| | (SEQ ID NO: 101) | | |
| www.iedb.org/epitope/190882 | NQNLRVAKISMPVNTPGQFE | 280 | 299 |
| | (SEQ ID NO: 102) | | |
| www.iedb.org/epitope/190885 | PCAQRCLQSCQQEPDDLKQK | 37 | 56 |
| | (SEQ ID NO: 103) | | |
| www.iedb.org/epitope/190893 | PINLREGEPDLSNNFGKLFE | 397 | 416 |
| | (SEQ ID NO: 104) | | |
| www.iedb.org/epitope/190895 | PQLQDLDMMLTCVEIKEGAL | 421 | 440 |
| | (SEQ ID NO: 105) | | |
| www.iedb.org/epitope/190907 | QEEENQGGKGPLLSILKAFN | 600 | 619 |
| | (SEQ ID NO: 106) | | |
| www.iedb.org/epitope/190909 | QKACESRCTKLEYDPRCVYD | 55 | 74 |
| | (SEQ ID NO: 107) | | |
| www.iedb.org/epitope/190946 | RDQSSYLQGFSRNTLEAAFN | 304 | 323 |
| | (SEQ ID NO: 108) | | |
| www.iedb.org/epitope/190947 | REEGGRWGPAGPREREREED | 109 | 128 |
| | (SEQ ID NO: 109) | | |
| www.iedb.org/epitope/190949 | RIVQIEAKPNTLV | 213 | 225 |
| | (SEQ ID NO: 110) | | |
| www.iedb.org/epitope/190952 | RKSFNLDEGHALRIPSGFIS | 253 | 272 |
| | (SEQ ID NO: 111) | | |
| www.iedb.org/epitope/190966 | RVLLEENAGGEQEERGQRRW | 331 | 350 |
| | (SEQ ID NO: 112) | | |
| www.iedb.org/epitope/190967 | RWSTRSSENNEGVIVKVSKE | 352 | 371 |
| | (SEQ ID NO: 113) | | |
| www.iedb.org/epitope/190971 | SEEEGDITNPINLREGEPDL | 385 | 404 |
| | (SEQ ID NO: 114) | | |
| www.iedb.org/epitope/190973 | SHVREETSRNNPFYFPSRRF | 163 | 182 |
| | (SEQ ID NO: 115) | | |
| www.iedb.org/epitope/190983 | SMPVNTPGQFEDFFPASSRD | 289 | 308 |
| | (SEQ ID NO: 116) | | |
| www.iedb.org/epitope/190987 | SPYQKKTENPCAQRCLQSCQ | 28 | 47 |
| | (SEQ ID NO: 117) | | |
| www.iedb.org/epitope/190988 | SSELHLLGFGINAENNHRIF | 523 | 542 |
| | (SEQ ID NO: 118) | | |
| www.iedb.org/epitope/190990 | SVSATHAKSSPYQKKTENPC | 19 | 38 |
| | (SEQ ID NO: 119) | | |
| www.iedb.org/epitope/190999 | TNQRSPPGERTRGRQPGDYD | 82 | 101 |
| | (SEQ ID NO: 120) | | |
| www.iedb.org/epitope/191006 | TVTVANGNNRKSFNLDEGHA | 244 | 263 |
| | (SEQ ID NO: 121) | | |
| www.iedb.org/epitope/191010 | VEIKEGALMLPHFNSKA | 433 | 449 |
| | (SEQ ID NO: 122) | | |
| www.iedb.org/epitope/191015 | VIDQIEKQAKDLAFPGSGEQ | 550 | 569 |
| | (SEQ ID NO: 123) | | |
| www.iedb.org/epitope/191029 | WRRPSHQQPRKIRPEGREGE | 136 | 155 |
| | (SEQ ID NO: 124) | | |
| www.iedb.org/epitope/191030 | WSTRSSENNEGVIVKVSKE | 353 | 371 |
| | (SEQ ID NO: 125) | | |
| www.iedb.org/epitope/191031 | YDDDRRQPRREEGGRWGPAG | 100 | 119 |
| | (SEQ ID NO: 126) | | |
| www.iedb.org/epitope/191032 | YDPRGHTGTTNQRSPPGERT | 73 | 92 |
| | (SEQ ID NO: 127) | | |
| www.iedb.org/epitope/420907 | EGSNREVRRYTARLK | 491 | 505 |
| | (SEQ ID NO: 128) | | |
| www.iedb.org/epitope/420973 | FGINAENNHRIFLAG | 531 | 545 |
| | (SEQ ID NO: 129) | | |
| www.iedb.org/epitope/421060 | GQFEDFFPASSRDQS | 296 | 310 |
| | (SEQ ID NO: 130) | | |
| www.iedb.org/epitope/421119 | IDQIEKQAKDLAFPG | 551 | 565 |
| | (SEQ ID NO: 131) | | |
| www.iedb.org/epitope/421485 | PHFNSKAMVIVVVNK | 446 | 460 |
| | (SEQ ID NO: 132) | | |
| www.iedb.org/epitope/540345 | ATVTVANGNNRKSFN | 243 | 257 |
| | (SEQ ID NO: 133) | | |
| www.iedb.org/epitope/540385 | EGDVFIMPAAHPVAI | 506 | 520 |
| | (SEQ ID NO: 134) | | |
| www.iedb.org/epitope/540386 | EHVEELTKHAKSVSK | 371 | 385 |
| | (SEQ ID NO: 135) | | |
| www.iedb.org/epitope/540387 | ENNHRIFLAGDKDNV | 536 | 550 |
| | (SEQ ID NO: 136) | | |
| www.iedb.org/epitope/540393 | EVKPDKKNPQLQDLD | 416 | 430 |
| | (SEQ ID NO: 137) | | |
| www.iedb.org/epitope/540406 | FQNLQNHRIVQIEAK | 206 | 220 |
| | (SEQ ID NO: 138) | | |
| www.iedb.org/epitope/540436 | GRIRVLQRFDQRSRQ | 191 | 205 |
| | (SEQ ID NO: 139) | | |
| www.iedb.org/epitope/540443 | HFVSARPQSQSQSPS | 581 | 595 |
| | (SEQ ID NO: 140) | | |
| www.iedb.org/epitope/540451 | HPVAINASSELHLLG | 516 | 530 |
| | (SEQ ID NO: 141) | | |
| www.iedb.org/epitope/540454 | HRIVQIEAKPNTLVL | 212 | 226 |
| | (SEQ ID NO: 142) | | |
| www.iedb.org/epitope/540516 | MMLTCVEIKEGALML | 431 | 445 |
| | (SEQ ID NO: 143) | | |
| www.iedb.org/epitope/540521 | NAEFNEIRRVLLEEN | 326 | 340 |
| | (SEQ ID NO: 144) | | |
| www.iedb.org/epitope/540540 | PDLSNNFGKLFEVKP | 405 | 419 |
| | (SEQ ID NO: 145) | | |
| www.iedb.org/epitope/540565 | QGGKGPLLSILKAFN | 612 | 626 |
| | (SEQ ID NO: 146) | | |
| www.iedb.org/epitope/540582 | RNNPFYFPSRRFSTR | 171 | 185 |
| | (SEQ ID NO: 147) | | |
| www.iedb.org/epitope/540616 | SYLQGFSRNTLEAAF | 311 | 325 |
| | (SEQ ID NO: 148) | | |
| www.iedb.org/epitope/836864 | DADNILVIQQGQATV | 231 | 245 |
| | (SEQ ID NO: 149) | | |
| www.iedb.org/epitope/837272 | FQNLQNHRIVQIEAR | 200 | 214 |
| | (SEQ ID NO: 150) | | |
| www.iedb.org/epitope/837401 | GRIRVLQRFDQRSKQ | 185 | 199 |
| | (SEQ ID NO: 151) | | |
| www.iedb.org/epitope/837599 | IPSGFISYILNRHDN | 266 | 280 |
| | (SEQ ID NO: 152) | | |
| www.iedb.org/epitope/838089 | PHFNSKAMVIVVINK | 443 | 457 |
| | (SEQ ID NO: 153) | | |
| www.iedb.org/epitope/838252 | RCTKLEYDPRCVYDT | 59 | 73 |
| | (SEQ ID NO: 154) | | |

Accordingly, in certain embodiments the epitopes comprising the tolerogenic nanoparticles described herein comprises one or more of the epitopes shown in Table 2, and/or the nucleic acid(s) comprising the tolerogenic nanoparticles described herein encode one or more of the epitopes shown in Table 2.

TABLE 3
Experimentally deposited and tested linear Ara h 2 epitopes in the IEDB for
humans.

| | | Starting | Ending |
|---|---|---|---|
| Epitope IRI | Sequence | Position | Position |
| www.iedb.org/epitope/1744 | AHASARQQWELQGDRRCQSQ | 36 | 55 |
| | (SEQ ID NO: 155) | | |
| www.iedb.org/epitope/2241 | AKLTILVALALFLLAAHASA | 21 | 40 |
| | (SEQ ID NO: 156) | | |
| www.iedb.org/epitope/2850 | ALQQIMENQSDRLQGRQQE | 127 | 145 |
| | (SEQ ID NO: 157) | | |
| www.iedb.org/epitope/3348 | ANLRPCEQHLMQKIQRDEDS | 38 | 57 |
| | (SEQ ID NO: 158) | | |
| www.iedb.org/epitope/6120 | CDLDVESGGRDRY | 146 | 158 |
| | (SEQ ID NO: 159) | | |
| www.iedb.org/epitope/6690 | CNELNEFENNQRCMCEALQQ | 111 | 130 |
| | (SEQ ID NO: 160) | | |
| www.iedb.org/epitope/10276 | DSYERDPYSPSQDPYSPSPY | 77 | 96 |
| | (SEQ ID NO: 161) | | |
| www.iedb.org/epitope/13187 | ELNEFENNQRCMCEALQQIM | 113 | 132 |
| | (SEQ ID NO: 162) | | |
| www.iedb.org/epitope/15608 | FENNQRCMCEALQQIMENQ | 117 | 135 |
| | (SEQ ID NO: 163) | | |
| www.iedb.org/epitope/16453 | FKRELRNLPQQCGLRAPQ | 148 | 165 |
| | (SEQ ID NO: 164) | | |
| www.iedb.org/epitope/16704 | FLLAAHASARQQWELQGDRR | 11 | 30 |
| | (SEQ ID NO: 165) | | |
| www.iedb.org/epitope/21092 | GLRAPQRCDLDVESGGRDRY | 139 | 158 |
| | (SEQ ID NO: 166) | | |
| www.iedb.org/epitope/22176 | GRQQEQQFKRELRNLPQQCG | 141 | 160 |
| | (SEQ ID NO: 167) | | |
| www.iedb.org/epitope/24258 | HLMQKIQRDEDSYERDPYS | 67 | 85 |
| | (SEQ ID NO: 168) | | |
| www.iedb.org/epitope/27480 | IMENQSDRLQGRQQEQQFKR | 131 | 150 |
| | (SEQ ID NO: 169) | | |
| www.iedb.org/epitope/33124 | KRELRNLPQQCGLRAPQRCD | 128 | 147 |
| | (SEQ ID NO: 170) | | |
| www.iedb.org/epitope/35674 | LERANLRPCEQHLMQKIQRD | 56 | 75 |
| | (SEQ ID NO: 171) | | |
| www.iedb.org/epitope/35897 | LFLLAAHASARQQWELQGDR | 31 | 50 |
| | (SEQ ID NO: 172) | | |
| www.iedb.org/epitope/38101 | LMQKIQRDEDSYERDPYSPS | 47 | 66 |
| | (SEQ ID NO: 173) | | |
| www.iedb.org/epitope/39033 | LRAPQRCDLDVESGGRDRY | 161 | 179 |
| | (SEQ ID NO: 174) | | |
| www.iedb.org/epitope/39150 | LRNLPQQCGLRAPQRCDLD | 152 | 170 |
| | (SEQ ID NO: 175) | | |
| www.iedb.org/epitope/39159 | LRPCEQHLMQKIQRDEDSYE | 61 | 80 |
| | (SEQ ID NO: 176) | | |
| www.iedb.org/epitope/40259 | LVALALFLLAAHASARQQWE | 26 | 45 |
| | (SEQ ID NO: 177) | | |
| www.iedb.org/epitope/49076 | PQQCGLRAPQRCDLDVESGG | 156 | 175 |
| | (SEQ ID NO: 178) | | |
| www.iedb.org/epitope/49484 | PSQDPYSPSPYDRRGAGSSQ | 65 | 84 |
| | (SEQ ID NO: 179) | | |
| www.iedb.org/epitope/50079 | PYDRRGAGSSQHQERCCNEL | 95 | 114 |
| | (SEQ ID NO: 180) | | |
| www.iedb.org/epitope/50407 | QCGLRAPQRCDLDVESGGRD | 158 | 177 |
| | (SEQ ID NO: 181) | | |
| www.iedb.org/epitope/50954 | QGRQQEQQFKRELRNLPQQC | 119 | 138 |
| | (SEQ ID NO: 182) | | |
| www.iedb.org/epitope/53290 | RCMCEALQQIMENQSDRLQ | 122 | 140 |
| | (SEQ ID NO: 183) | | |
| www.iedb.org/epitope/53291 | RCMCEALQQIMENQSDRLQG | 122 | 141 |
| | (SEQ ID NO: 184) | | |
| www.iedb.org/epitope/55479 | RQQWELQGDRRCQSQLERAN | 41 | 60 |
| | (SEQ ID NO: 185) | | |
| www.iedb.org/epitope/55536 | RRCQSQLERANLRPCEQHLM | 50 | 69 |
| | (SEQ ID NO: 186) | | |
| www.iedb.org/epitope/57267 | SDRLQGRQQEQQFKRELRNL | 136 | 155 |
| | (SEQ ID NO: 187) | | |
| www.iedb.org/epitope/60453 | SQHQERCCNELNEFENNQRC | 104 | 123 |
| | (SEQ ID NO: 188) | | |
| www.iedb.org/epitope/150825 | ANLRPCEQHLM | 38 | 48 |
| | (SEQ ID NO: 189) | | |
| www.iedb.org/epitope/150838 | ELNEFENNQRCM | 92 | 103 |
| | (SEQ ID NO: 190) | | |
| www.iedb.org/epitope/150839 | ELNEFENNQRCMCEALQ | 92 | 108 |
| | (SEQ ID NO: 191) | | |
| www.iedb.org/epitope/150841 | FENNQRCMCEALQ | 96 | 108 |
| | (SEQ ID NO: 192) | | |
| www.iedb.org/epitope/150879 | RELRNLPQQCGLRA | 129 | 142 |
| | (SEQ ID NO: 193) | | |
| www.iedb.org/epitope/150886 | SQLERANLRPCEQ | 33 | 45 |
| | (SEQ ID NO: 194) | | |
| www.iedb.org/epitope/150887 | SQLERANLRPCEQHLM | 33 | 48 |
| | (SEQ ID NO: 195) | | |
| www.iedb.org/epitope/178524 | ALQQIMENQSDRLQGRQQEQ | 106 | 125 |
| | (SEQ ID NO: 196) | | |
| www.iedb.org/epitope/178630 | CQSQLERANLRPCEQHLMQK | 31 | 50 |
| | (SEQ ID NO: 197) | | |
| www.iedb.org/epitope/178685 | DPYSPSQDPYSPSPYDRRGA | 61 | 80 |
| | (SEQ ID NO: 198) | | |
| www.iedb.org/epitope/178690 | DRLQGRQQEQQFKRELRNLP | 116 | 135 |
| | (SEQ ID NO: 199) | | |
| www.iedb.org/epitope/178692 | DRRGAGSSQHQERCCNELNE | 76 | 95 |
| | (SEQ ID NO: 200) | | |
| www.iedb.org/epitope/178767 | ERANLRPCEQHLMQKIQRDE | 36 | 55 |
| | (SEQ ID NO: 201) | | |
| www.iedb.org/epitope/178803 | FENNQRCMCEALQQIMENQS | 96 | 115 |
| | (SEQ ID NO: 202) | | |
| www.iedb.org/epitope/178938 | GSSQHQERCCNELNEFENNQ | 81 | 100 |
| | (SEQ ID NO: 203) | | |
| www.iedb.org/epitope/178961 | HASARQQWELQGDRRCQSQL | 16 | 35 |
| | (SEQ ID NO: 204) | | |
| www.iedb.org/epitope/178965 | HLMQKIQRDEDSYERDPYSP | 46 | 65 |
| | (SEQ ID NO: 205) | | |
| www.iedb.org/epitope/179034 | IQRDEDSYERDPYSPSQDPY | 51 | 70 |
| | (SEQ ID NO: 206) | | |
| www.iedb.org/epitope/179200 | LRNLPQQCGLRAPQRCDLDV | 131 | 150 |
| | (SEQ ID NO: 207) | | |
| www.iedb.org/epitope/179248 | MENQSDRLQGRQQEQQFKRE | 111 | 130 |
| | (SEQ ID NO: 208) | | |
| www.iedb.org/epitope/179263 | MLTILVALALFLLAAHASAR | 1 | 20 |
| | (SEQ ID NO: 209) | | |
| www.iedb.org/epitope/179296 | NELNEFENNQRCMCEALQQI | 91 | 110 |
| | (SEQ ID NO: 210) | | |
| www.iedb.org/epitope/179373 | QERCCNELNEFENNQRCMCE | 86 | 105 |
| | (SEQ ID NO: 211) | | |
| www.iedb.org/epitope/179375 | QFKRELRNLPQQCGLRAPQR | 126 | 145 |
| | (SEQ ID NO: 212) | | |
| www.iedb.org/epitope/179376 | QGDRRCQSQLERANLRPCEQ | 26 | 45 |
| | (SEQ ID NO: 213) | | |
| www.iedb.org/epitope/179386 | QQCGLRAPQRCDLDVESGGR | 136 | 155 |
| | (SEQ ID NO: 214) | | |
| www.iedb.org/epitope/179390 | QQWELQGDRRCQSQLERANL | 21 | 40 |
| | (SEQ ID NO: 215) | | |
| www.iedb.org/epitope/179407 | RAPQRCDLDVESGGRDRY | 141 | 158 |
| | (SEQ ID NO: 216) | | |
| www.iedb.org/epitope/179441 | RPCEQHLMQKIQRDEDSYER | 41 | 60 |
| | (SEQ ID NO: 217) | | |
| www.iedb.org/epitope/179446 | RQQEQQFKRELRNLPQQCGL | 121 | 140 |
| | (SEQ ID NO: 218) | | |
| www.iedb.org/epitope/179493 | SGGRDRY | 152 | 158 |
| | (SEQ ID NO: 219) | | |
| www.iedb.org/epitope/179519 | SPSPYDRRGAGSSQHQERCC | 71 | 90 |
| | (SEQ ID NO: 220) | | |
| www.iedb.org/epitope/179520 | SQDPYSPSPYDRRGAGSSQH | 66 | 85 |
| | (SEQ ID NO: 221) | | |
| www.iedb.org/epitope/179605 | VALALFLLAAHASARQQWEL | 6 | 25 |
| | (SEQ ID NO: 222) | | |
| www.iedb.org/epitope/769859 | CNELNEFENNQRCMCEALQQI | 90 | 110 |
| | (SEQ ID NO: 223) | | |
| www.iedb.org/epitope/836742 | ALFLLAAHASARQQW | 11 | 25 |
| | (SEQ ID NO: 224) | | |
| www.iedb.org/epitope/836818 | ARQQWELQGDRRCQS | 21 | 35 |
| | (SEQ ID NO: 225) | | |

Accordingly, in certain embodiments the epitopes comprising the tolerogenic nanoparticles described herein comprises one or more of the epitopes shown in Table 3, and/or the nucleic acid(s) comprising the tolerogenic nanoparticles described herein encode one or more of the epitopes shown in Table 3.

TABLE 4
Experimentally deposited/tested Ara h 3 epitopes in the IEDB for humans. Epitope
is linear peptide.

| | | Starting | Ending |
|---|---|---|---|
| Epitope IRI | Sequence | Position | Position |
| www.iedb.org/epitope/71559 | VTVRGGLRILSPDRK | 299 | 313 |
| | (SEQ ID NO: 226) | | |
| www.iedb.org/epitope/99237 | EYVAFKTDSRPSIAN | 465 | 479 |
| | (SEQ ID NO: 227) | | |
| www.iedb.org/epitope/99408 | LAGENSVIDNLPEEV | 480 | 494 |
| | (SEQ ID NO: 228) | | |
| www.iedb.org/epitope/99560 | QEFLRYQQQSRQSRR | 204 | 218 |
| | (SEQ ID NO: 229) | | |
| www.iedb.org/epitope/105758 | EQGAIVTVRGGLRIL | 294 | 308 |
| | (SEQ ID NO: 230) | | |
| www.iedb.org/epitope/105929 | LILRWLGLSAEYGNL | 385 | 399 |
| | (SEQ ID NO: 231) | | |
| www.iedb.org/epitope/106026 | QARQLKNNNPFKFFV | 486 | 500 |
| | (SEQ ID NO: 232) | | |
| www.iedb.org/epitope/106104 | SGFTPEFLAQAFQVD | 264 | 278 |
| | (SEQ ID NO: 233) | | |
| www.iedb.org/epitope/106108 | SIIYALRGRAHVQVV | 415 | 429 |
| | (SEQ ID NO: 234) | | |
| www.iedb.org/epitope/836708 | AFQVDDRQIVQNLRG | 274 | 288 |
| | (SEQ ID NO: 235) | | |
| www.iedb.org/epitope/837086 | EFLAQAFQVDDRQIV | 269 | 283 |
| | (SEQ ID NO: 236) | | |
| www.iedb.org/epitope/837195 | EYLAFKTDSRPSIAN | 446 | 460 |
| | (SEQ ID NO: 237) | | |
| www.iedb.org/epitope/837291 | FVPHYTLNAHTIVVA | 386 | 400 |
| | (SEQ ID NO: 238) | | |
| www.iedb.org/epitope/837414 | GSRPHYRQISPRVRG | 216 | 230 |
| | (SEQ ID NO: 239) | | |
| www.iedb.org/epitope/837502 | HVQVVDSNGNRVYDE | 406 | 420 |
| | (SEQ ID NO: 240) | | |
| www.iedb.org/epitope/838062 | NTNAHSIIYALRGRA | 410 | 424 |
| | (SEQ ID NO: 241) | | |
| www.iedb.org/epitope/838155 | QAGENSIIDNLPEEV | 461 | 475 |
| | (SEQ ID NO: 242) | | |
| www.iedb.org/epitope/838172 | QEFLQHAFQVDRQTV | 246 | 260 |
| | (SEQ ID NO: 243) | | |
| www.iedb.org/epitope/838229 | QSDNFEYVAFKTDSR | 460 | 474 |
| | (SEQ ID NO: 244) | | |
| www.iedb.org/epitope/838385 | RRPFYSNAPLEIYVQ | 81 | 95 |
| | (SEQ ID NO: 245) | | |
| www.iedb.org/epitope/838472 | SGFAQEFLQHAFQVD | 242 | 256 |
| | (SEQ ID NO: 246) | | |
| www.iedb.org/epitope/838558 | SRLVLRRNALRRPFY | 71 | 85 |
| | (SEQ ID NO: 247) | | |
| www.iedb.org/epitope/838678 | TIVVALNGRAHVQVV | 396 | 410 |
| | (SEQ ID NO: 248) | | |
| www.iedb.org/epitope/838688 | TLNAHTIVVALNGRA | 391 | 405 |
| | (SEQ ID NO: 249) | | |

Accordingly, in certain embodiments the epitopes comprising the tolerogenic nanoparticles described herein comprises one or more of the epitopes shown in Table 4, and/or the nucleic acid(s) comprising the tolerogenic nanoparticles described herein encode one or more of the epitopes shown in Table 4.

Because there is no deposited epitope information for another major peanut allergen, Ara h 6, we conducted our own epitope search for Ara-h6, using the IEDB resource. The search included the use of the complete Ara h 6 peptide sequence, as well as using the full HLA reference set for prediction making of binding interactions with HLA-DR, HLA-DP, or HLA-DQ. We selected the upper 5% of predicted 15-mer sequences, which were ranked according to the affinity binding interactions with these HLA constructs (Table 5).

TABLE 5
In-house human T cell epitope predictions for Ara h 6 by the IEDB database.

| | | | | | Percentile |
|---|---|---|---|---|---|
| Allele | start | end | length | Peptide | rank |
| HLA-DRB1*01: 01 | 3 | 17 | 15 | KSTILVALLALVLVA | 0.01 |
| | | | | (SEQ ID NO: 250) | |
| HLA-DRB1*01: 01 | 4 | 18 | 15 | STILVALLALVLVAH | 0.01 |
| | | | | (SEQ ID NO: 251) | |
| HLA-DRB1*01: 01 | 2 | 16 | 15 | AKSTILVALLALVLV | 0.1 |
| | | | | (SEQ ID NO: 252) | |
| HLA-DRB1*01: 01 | 1 | 15 | 15 | MAKSTILVALLALVL | 0.1 |
| | | | | (SEQ ID NO: 253) | |
| HLA-DRB1*01: 01 | 5 | 19 | 15 | TILVALLALVLVAHA | 0.1 |
| | | | | (SEQ ID NO: 254) | |
| HLA-DRB1*01: 01 | 6 | 20 | 15 | ILVALLALVLVAHAS | 0.56 |
| | | | | (SEQ ID NO: 255) | |
| HLA-DRB1*01: 01 | 7 | 21 | 15 | LVALLALVLVAHASA | 0.67 |
| | | | | (SEQ ID NO: 256) | |
| HLA-DRB5*01: 01 | 12 | 26 | 15 | ALVLVAHASAMRRER | 0.81 |
| | | | | (SEQ ID NO: 257) | |
| HLA- | 3 | 17 | 15 | KSTILVALLALVLVA | 0.81 |
| DPA1*03: 01/ | | | | (SEQ ID NO: 258) | |
| DPB1*04: 02 | | | | | |
| HLA-DRB5*01: 01 | 11 | 25 | 15 | LALVLVAHASAMRRE | 0.84 |
| | | | | (SEQ ID NO: 259) | |
| HLA-DRB5*01: 01 | 10 | 24 | 15 | LLALVLVAHASAMRR | 0.84 |
| | | | | (SEQ ID NO: 260) | |
| HLA-DRB5*01: 01 | 13 | 27 | 15 | LVLVAHASAMRRERG | 0.84 |
| | | | | (SEQ ID NO: 261) | |
| HLA- | 2 | 16 | 15 | AKSTILVALLALVLV | 0.85 |
| DPA1*03: 01/ | | | | (SEQ ID NO: 262) | |
| DPB1*04: 02 | | | | | |
| HLA- | 12 | 26 | 15 | ALVLVAHASAMRRER | 0.87 |
| DPA1*02: 01/ | | | | (SEQ ID NO: 263) | |
| DPB1*14: 01 | | | | | |
| HLA- | 1 | 15 | 15 | MAKSTILVALLALVL | 0.99 |
| DPA1*03: 01/ | | | | (SEQ ID NO: 264) | |
| DPB1*04: 02 | | | | | |
| HLA- | 11 | 25 | 15 | LALVLVAHASAMRRE | 1.1 |
| DPA1*02: 01/ | | | | (SEQ ID NO: 265) | |
| DPB1*14: 01 | | | | | |
| HLA- | 4 | 18 | 15 | STILVALLALVLVAH | 1.1 |
| DPA1*03: 01/ | | | | (SEQ ID NO: 266) | |
| DPB1*04: 02 | | | | | |
| HLA-DRB1*08: 02 | 9 | 23 | 15 | ALLALVLVAHASAMR | 1.3 |
| | | | | (SEQ ID NO: 267) | |
| HLA- | 13 | 27 | 15 | LVLVAHASAMRRERG | 1.3 |
| DPA1*02: 01/ | | | | (SEQ ID NO: 268) | |
| DPB1*14: 01 | | | | | |
| HLA- | 10 | 24 | 15 | LLALVLVAHASAMRR | 1.4 |
| DPA1*02: 01/ | | | | (SEQ ID NO: 269) | |
| DPB1*14: 01 | | | | | |
| HLA- | 5 | 19 | 15 | TILVALLALVLVAHA | 1.4 |
| DPA1*03: 01/ | | | | (SEQ ID NO: 270) | |
| DPB1*04: 02 | | | | | |
| HLA-DRB5*01: 01 | 14 | 28 | 15 | VLVAHASAMRRERGR | 1.4 |
| | | | | (SEQ ID NO: 271) | |
| HLA- | 5 | 19 | 15 | TILVALLALVLVAHA | 1.7 |
| DQA1*01: 02/ | | | | (SEQ ID NO: 272) | |
| DQB1*06: 02 | | | | | |
| HLA-DRB1*08: 02 | 8 | 22 | 15 | VALLALVLVAHASAM | 1.7 |
| | | | | (SEQ ID NO: 273) | |
| HLA- | 6 | 20 | 15 | ILVALLALVLVAHAS | 1.8 |
| DQA1*01: 02/ | | | | (SEQ ID NO: 274) | |
| DQB1*06: 02 | | | | | |
| HLA-DRB1*08: 02 | 10 | 24 | 15 | LLALVLVAHASAMRR | 1.9 |
| | | | | (SEQ ID NO: 275) | |
| HLA- | 7 | 21 | 15 | LVALLALVLVAHASA | 1.9 |
| DQA1*01: 02/ | | | | (SEQ ID NO: 276) | |
| DQB1*06: 02 | | | | | |
| HLA- | 13 | 27 | 15 | LVLVAHASAMRRERG | 1.9 |
| DQA1*01: 02/ | | | | (SEQ ID NO: 277) | |
| DQB1*06: 02 | | | | | |
| HLA-DRB1*08: 02 | 11 | 25 | 15 | LALVLVAHASAMRRE | 2 |
| | | | | (SEQ ID NO: 278) | |
| HLA- | 8 | 22 | 15 | VALLALVLVAHASAM | 2 |
| DQA1*01: 02/ | | | | (SEQ ID NO: 279) | |
| DQB1*06: 02 | | | | | |
| HLA-DRB3*02: 02 | 12 | 26 | 15 | ALVLVAHASAMRRER | 2.1 |
| | | | | (SEQ ID NO: 280) | |
| HLA-DRB3*02: 02 | 11 | 25 | 15 | LALVLVAHASAMRRE | 2.1 |
| | | | | (SEQ ID NO: 281) | |
| HLA- | 3 | 17 | 15 | KSTILVALLALVLVA | 2.3 |
| DPA1*02: 01/ | | | | (SEQ ID NO: 282) | |
| DPB1*01: 01 | | | | | |
| HLA-DRB3*02: 02 | 10 | 24 | 15 | LLALVLVAHASAMRR | 2.3 |
| | | | | (SEQ ID NO: 283) | |
| HLA- | 2 | 16 | 15 | AKSTILVALLALVLV | 2.4 |
| DPA1*02: 01/ | | | | (SEQ ID NO: 284) | |
| DPB1*01: 01 | | | | | |
| HLA-DRB1*15: 01 | 2 | 16 | 15 | AKSTILVALLALVLV | 2.4 |
| | | | | (SEQ ID NO: 285) | |
| HLA-DRB1*15: 01 | 6 | 20 | 15 | ILVALLALVLVAHAS | 2.4 |
| | | | | (SEQ ID NO: 286) | |
| HLA-DRB1*15: 01 | 3 | 17 | 15 | KSTILVALLALVLVA | 2.4 |
| | | | | (SEQ ID NO: 287) | |
| HLA-DRB1*15: 01 | 7 | 21 | 15 | LVALLALVLVAHASA | 2.4 |
| | | | | (SEQ ID NO: 288) | |
| HLA-DRB1*15: 01 | 1 | 15 | 15 | MAKSTILVALLALVL | 2.4 |
| | | | | (SEQ ID NO: 289) | |
| HLA-DRB1*15: 01 | 4 | 18 | 15 | STILVALLALVLVAH | 2.4 |
| | | | | (SEQ ID NO: 290) | |
| HLA-DRB1*15: 01 | 5 | 19 | 15 | TILVALLALVLVAHA | 2.4 |
| | | | | (SEQ ID NO: 291) | |
| HLA- | 14 | 28 | 15 | VLVAHASAMRRERGR | 2.4 |
| DQA1*01: 02/ | | | | (SEQ ID NO: 292) | |
| DQB1*06: 02 | | | | | |
| HLA-DRB3*02: 02 | 13 | 27 | 15 | LVLVAHASAMRRERG | 2.5 |
| | | | | (SEQ ID NO: 293) | |
| HLA- | 4 | 18 | 15 | STILVALLALVLVAH | 2.5 |
| DPA1*02: 01/ | | | | (SEQ ID NO: 294) | |
| DPB1*01: 01 | | | | | |
| HLA-DRB1*01: 01 | 8 | 22 | 15 | VALLALVLVAHASAM | 2.6 |
| | | | | (SEQ ID NO: 295) | |
| HLA- | 6 | 20 | 15 | ILVALLALVLVAHAS | 2.7 |
| DPA1*03: 01/ | | | | (SEQ ID NO: 296) | |
| DPB1*04: 02 | | | | | |
| HLA- | 1 | 15 | 15 | MAKSTILVALLALVL | 2.7 |
| DQA1*01: 02/ | | | | (SEQ ID NO: 297) | |
| DQB1*06: 02 | | | | | |
| HLA- | 12 | 26 | 15 | ALVLVAHASAMRRER | 2.8 |
| DQA1*01: 02/ | | | | (SEQ ID NO: 298) | |
| DQB1*06: 02 | | | | | |
| HLA-DRB1*01: 01 | 9 | 23 | 15 | ALLALVLVAHASAMR | 2.9 |
| | | | | (SEQ ID NO: 299) | |
| HLA-DRB1*01: 01 | 10 | 24 | 15 | LLALVLVAHASAMRR | 2.9 |
| | | | | (SEQ ID NO: 300) | |
| HLA- | 9 | 23 | 15 | ALLALVLVAHASAMR | 3 |
| DQA1*01: 02/ | | | | (SEQ ID NO: 301) | |
| DQB1*06: 02 | | | | | |
| HLA- | 1 | 15 | 15 | MAKSTILVALLALVL | 3 |
| DPA1*02: 01/ | | | | (SEQ ID NO: 302) | |
| DPB1*01: 01 | | | | | |
| HLA- | 14 | 28 | 15 | VLVAHASAMRRERGR | 3.2 |
| DPA1*02: 01/ | | | | (SEQ ID NO: 303) | |
| DPB1*14: 01 | | | | | |
| HLA-DRB1*07: 01 | 2 | 16 | 15 | AKSTILVALLALVLV | 3.3 |
| | | | | (SEQ ID NO: 304) | |
| HLA-DRB1*07: 01 | 3 | 17 | 15 | KSTILVALLALVLVA | 3.3 |
| | | | | (SEQ ID NO: 305) | |
| HLA-DRB1*01: 01 | 11 | 25 | 15 | LALVLVAHASAMRRE | 3.3 |
| | | | | (SEQ ID NO: 306) | |
| HLA-DRB1*08: 02 | 7 | 21 | 15 | LVALLALVLVAHASA | 3.3 |
| | | | | (SEQ ID NO: 307) | |
| HLA-DRB1*07: 01 | 1 | 15 | 15 | MAKSTILVALLALVL | 3.3 |
| | | | | (SEQ ID NO: 308) | |
| HLA- | 4 | 18 | 15 | STILVALLALVLVAH | 3.5 |
| DQA1*01: 02/ | | | | (SEQ ID NO: 309) | |
| DQB1*06: 02 | | | | | |
| HLA- | 2 | 16 | 15 | AKSTILVALLALVLV | 3.8 |
| DPA1*02: 01/ | | | | (SEQ ID NO: 310) | |
| DPB1*05: 01 | | | | | |
| HLA- | 3 | 17 | 15 | KSTILVALLALVLVA | 3.8 |
| DPA1*02: 01/ | | | | (SEQ ID NO: 311) | |
| DPB1*05: 01 | | | | | |
| HLA- | 4 | 18 | 15 | STILVALLALVLVAH | 3.8 |
| DPA1*02: 01/ | | | | (SEQ ID NO: 312) | |
| DPB1*05: 01 | | | | | |
| HLA- | 5 | 19 | 15 | TILVALLALVLVAHA | 3.8 |
| DPA1*02: 01/ | | | | (SEQ ID NO: 313) | |
| DPB1*01: 01 | | | | | |
| HLA-DRB1*07: 01 | 5 | 19 | 15 | TILVALLALVLVAHA | 3.8 |
| | | | | (SEQ ID NO: 314) | |
| HLA- | 15 | 29 | 15 | LVAHASAMRRERGRQ | 3.9 |
| DQA1*01: 02/ | | | | (SEQ ID NO: 315) | |
| DQB1*06: 02 | | | | | |
| HLA-DRB5*01: 01 | 15 | 29 | 15 | LVAHASAMRRERGRQ | 4.1 |
| | | | | (SEQ ID NO: 316) | |
| HLA- | 3 | 17 | 15 | KSTILVALLALVLVA | 4.2 |
| DQA1*05: 01/ | | | | (SEQ ID NO: 317) | |
| DQB1*02: 01 | | | | | |
| HLA- | 2 | 16 | 15 | AKSTILVALLALVLV | 4.3 |
| DQA1*05: 01/ | | | | (SEQ ID NO: 318) | |
| DQB1*02: 01 | | | | | |
| HLA- | 1 | 15 | 15 | MAKSTILVALLALVL | 4.5 |
| DQA1*05: 01/ | | | | (SEQ ID NO: 319) | |
| DQB1*02: 01 | | | | | |
| HLA- | 9 | 23 | 15 | ALLALVLVAHASAMR | 4.7 |
| DPA1*02: 01/ | | | | (SEQ ID NO: 320) | |
| DPB1*14: 01 | | | | | |
| HLA- | 11 | 25 | 15 | LALVLVAHASAMRRE | 4.7 |
| DQA1*01: 02/ | | | | (SEQ ID NO: 321) | |
| DQB1*06: 02 | | | | | |
| HLA- | 4 | 18 | 15 | STILVALLALVLVAH | 4.7 |
| DQA1*05: 01/ | | | | (SEQ ID NO: 322) | |
| DQB1*02: 01 | | | | | |
| HLA-DRB1*15: 01 | 10 | 24 | 15 | LLALVLVAHASAMRR | 4.8 |
| | | | | (SEQ ID NO: 323) | |
| HLA- | 6 | 20 | 15 | ILVALLALVLVAHAS | 4.9 |
| DPA1*02: 01/ | | | | (SEQ ID NO: 324) | |
| DPB1*01: 01 | | | | | |
| HLA-DRB1*15: 01 | 11 | 25 | 15 | LALVLVAHASAMRRE | 5 |
| | | | | (SEQ ID NO: 325) | |

Accordingly, in certain embodiments the epitopes comprising the tolerogenic nanoparticles described herein comprises one or more of the epitopes shown in Table 5, and/or the nucleic acid(s) comprising the tolerogenic nanoparticles described herein encode one or more of the epitopes shown in Table 5.

Further refinement of the epitope predictions in Tables 2-5 was performed by considering a number of data resources and refinement techniques. Prickett et al. (2013) Clinical & Experimental Allergy, 43:684-97, claim to have generated the first report of peptide sequences that may serve as CD4+T cell epitopes for Ara h 1. This led to the identification and characterization of 10 HLA-promiscuous CD4+T-cell epitopes, which were used to assign lead Ara h 1 peptides that can be considered as candidates for therapeutic intervention. The authors selected nine 20-mer sequences to assess CD4+T-cell responses, using PBMC from 18 peanut-allergic subjects with diverse HLA alleles, typical for a Caucasian population. The authors further validated the 20-mer selection through the study of T cell responses in an additional 18 peanut-allergic subjects. Collectively, the response rate was 92%. The 20 mer responses were further refined by using overlapping peptides to identify shorter sequences, varying from 6-19 aa (see, e.g., Prickett et al. Table 3). This provided a prediction for 7 lead Ara-h1 peptide sequences as potential therapeutic leads (see, e.g., Table 6 below).

TABLE 6
Predicted CD4+ T cell epitopes for Ara h1.

| Epitope | Sequence | Core Sequence |
|---|---|---|
| 1 | FQNLQNHRIVQIEAKPNTLV | |
| | (SEQ ID NO: 326) | |
| 2 | WSTRSSENNEGVIVKVSKE | NEGVIVKVSK |
| | (SEQ ID NO: 327) | (SEQ ID NO: 328) |
| 3 | NNFGKLFEVKPDKKNPQLQ | |
| | (SEQ ID NO: 329) | |
| 4 | VEIKEGALMLPHENSKA | EGALML |
| | (SEQ ID NO: 330) | (SEQ ID NO: 331) |
| 5 | ALMLPHFNSKAMVIVVV | PHFNSKAMVIV |
| | (SEQ ID NO: 332) | (SEQ ID NO: 333) |
| 6 | KAMVIVVVNKGTGNLELVAV | |
| | (SEQ ID NO: 334) | |
| 7 | GDVFIMPAAHPVAINASS | IMPAAHP |
| | (SEQ ID NO: 335) | (SEQ ID NO: 336) |

Accordingly, in certain embodiments the epitopes comprising the tolerogenic nanoparticles described herein comprises one or more of the epitopes shown in Table 6, and/or the nucleic acid(s) comprising the tolerogenic nanoparticles described herein encode one or more of the epitopes shown in Table 6.

Pricket et al. (Table 3 in their publication), show the overlap of the 7 lead epitopes with the 20-mer sequences, originally used in T cell proliferation studies, as well as employing HLA-DR tetramers. In addition, the authors confirmed that 5 of these peptides could be presented by HLA molecules other than the molecules identified by tetramer mapping. Three of these epitopes (353-371, 436-452 and 442-458) were unique in their published study and could also be presented by HLA-DQ and HLA-DR molecules. The identification of HLA-DQ-restricted epitopes is of particular therapeutic benefit as these alleles are less variable and more prevalent in mixed populations than HLA-DR alleles. Tables 2-5, above, can also be used to identify additional DQ binding interactions.

Prickett et al. (2011) J. Allergy. Clin. Immunol. 127, 608-615 described experiments in which the authors selected 4 “dominant” Ara-h2 20-mers, premised on soliciting T-cell proliferation in PBMC from 16 HLA-diverse peanut allergic subjects. The dominant 20-mer sequences were originally studied by Stanley et al. (1997) Arch. Biochem. Biophys. 342:244-53), who described an elaborate series of 20-mer Ara h2 peptides. Prickett et al performed a proliferation study, using these sequences to identify 4×20-mer peptides, which were then used to identify core CD4⁺ peptide sequences. This led to the identification of 5 HLA-diverse T-cell epitopes that could potentially be used for T-cell targeted immunotherapy for peanut allergy. The mapping of these core T cell epitopes within the dominant 20-mer Ara h2 peptides have been identified in FIG. 2 from the same manuscript and are shown in Table 7, below.

TABLE 7
Core HLA-diverse T cell epitopes.

| Epitope | Sequence | Core Sequence |
|---|---|---|
| 1 | RRCQ SQLFRANLRPCEQHLM | LFRANLRPC |
| | (SEQ ID NO: 337) | (SEQ ID NO: 338) |
| 2 | ELNEFENNQRCM CEALQQIM | ELNEFENNQRCM |
| | (SEQ ID NO: 339) | (SEQ ID NO: 340) |
| 3 | K RFLRNLPQQCGLRA PQRCD | RFLRNLPQQCGL |
| | (SEQ ID NO: 341) | (SEQ ID NO: 342) |
| 4 | RRCQSQLER ANLRPCEQHLM | NLRPCEQHLM |
| | (SEQ ID NO: 343) | (SEQ ID NO: 344) |
| 5 | ELNEFENNQRCMCEALQ QIM | ENNQRCMCEA |
| | (SEQ ID NO: 345) | (SEQ ID NO: 346) |

Accordingly, in certain embodiments the epitopes comprising the tolerogenic nanoparticles described herein comprises one or more of the epitopes shown in Table 7, and/or the nucleic acid(s) comprising the telerogenic nanoparticles described herein encode one or more of the epitopes shown in Table 7.

Glaspole et al. (2005) Allergy, 2005:60: 35-40 mapped the T-cell epitopes of the major peanut allergen, Ara h 2, with a view to developing lead candidates for T-cell epitope vaccines. These epitopes were identified by the performance of proliferation and cytokine release studies, using a series of overlapping 20 mer peptides and a panel of 8 peanut-specific CD4⁺ T-cell lines from 8 human subjects. Two highly immunogenic T-cell response regions were identified, corresponding to aa 19-47 and 73-119. These epitopes were regarded as potentially safe therapeutic targets for use in a vaccine against peanut allergy.

Pascal et al. (2012) Clin. & Exp. Allergy, 43:116-127, identified four dominant Ara h 2 regions in response to long peptide sequences, used for identifying candidates for peptide-based immunotherapy. Shorter peptides with overlapping aa sequences were then studied for inducing PBMC proliferation and Th2-dominant cytokine production. Computer-assisted epitope mapping was used to confirm nine 9-mer MHC-II binding T cell epitopes for immunotherapy, as shown in Table 4 in the publication, and Table 8, below.

TABLE 8
Ara h2 epitopes.

| Ara H2 Region 1 | Ara h2 Region III | Ara h2 Region IV |
|---|---|---|
| (aa 7-23) | (aa 91-117) | (aa 126-141_{—} |
| aa 9-17 | aa 101-109 | aa 126-134 |
| aa 10-18 | aa 102-110 | aa 130-138 |
| aa 11-19 | aa 109-117 | |
| aa 12-20 | | |
| From Glaspole et al. (2005): | aa 19-47 | |
| | Aa 73-119 | |

Accordingly, in certain embodiments the epitopes comprising the tolerogenic nanoparticles described herein comprises one or more of the epitopes shown in Table 8, and/or the nucleic acid(s) comprising the tolerogenic nanoparticles described herein encode one or more of the epitopes shown in Table 8.

The Ramesh et al. (2016) J. Allergy Clin. Immunol. 137:1764-1771, authors sought to identify candidate Ara h 1 peptides displaying promiscuous MHC-II binding and ability to induce TH2 cytokine production by T cells from peanut-allergic subjects. Among a possible 600 overlapping 15-mer peptides, 36 were identified through in silico predictions and MHC class II binding assays. These peptide sequences are shown in Table 9, below, which was further adapted by showing calculations for p1 and GRAVY values (which predict loading capacity in our PLGA nanoparticles). These peptides were used by Ramesh et al. in proliferation assays as well as to study basophil degranulation (demonstrating lack of IgE crosslinking). This analysis led to the identification of 4 Ara-h1 vaccine candidates, which are highlighted in bold.

TABLE 9
Candidate Ara h1 peptides that display promiscuous MHC-II binding and ability to
induce TH2 cytokine production by T cells from peanut-allergic subjects.

| Amino | | | | | |
|---|---|---|---|---|---|
| Acids | Sequence | GRAVY | pI | Charge | Attribute |
| 16-30 | VLASVSATHAKSSPY | 0.247 | 8.57 | 2 | Basic |
| | (SEQ ID NO: 347) | | | | |
| 61-75 | RCTKLEYDPRCVYDP | −1.127 | 6.11 | 0 | Neutral |
| | (SEQ ID NO: 348) | | | | |
| 171-185 | RNNPFYFPSRRFSTR | −1.560 | 12.00 | 4 | Basic |
| | (SEQ ID NO: 349) | | | | |
| 191-205 | GRIRVLQRFDQRSRQ | −1.493 | 12.18 | 4 | Basic |
| | (SEQ ID NO: 350) | | | | |
| 196-210 | LQRFDQRSRQFQNLQ | −1.707 | 11.70 | 2 | Basic |
| | (SEQ ID NO: 351) | | | | |
| 201-215 | QRSRQFQNLQNHRIV | −1.547 | 12.30 | 4 | Basic |
| | (SEQ ID NO: 352) | | | | |
| 206-220 | FQNLQNHRIVQIEAK | −0.733 | 8.75 | 2 | Basic |
| | (SEQ ID NO: 353) | | | | |
| 212-226 | HRIVQIEAKPNTLVL | 0.160 | 8.75 | 2 | Basic |
| | (SEQ ID NO: 354) | | | | |
| 233-247 | DNILVIQQGQATVTV | 0.527 | 3.80 | −1 | Acidic |
| | (SEQ ID NO: 355) | | | | |
| 243+257 | ATVTVANGNNRKSFN | −0.680 | 11.00 | 2 | Basic |
| | (SEQ ID NO: 356) | | | | |
| 253-267 | RKSFNLDEGHALRIP | −0.847 | 8.75 | 2 | Basic |
| | (SEQ ID NO: 357) | | | | |
| 271-285 | ISYILNRHDNQNLRV | −0.733 | 8.75 | 2 | Basic |
| | (SEQ ID NO: 358) | | | | |
| 280-294 | NQNLRVAKISMPVNT | −0.340 | 11.00 | 2 | Basic |
| | (SEQ ID NO: 359) | | | | |
| 296-310 | GQFEDFFPASSRDQS | −1.080 | 4.03 | −2 | Acidic |
| | (SEQ ID NO: 360) | | | | |
| 311-325 | SYLQGFSRNTLEAAF | −0.147 | 5.72 | 0 | Neutral |
| | (SEQ ID NO: 361) | | | | |
| 321-335 | LEAAFNAEFNEIRRV | −0.200 | 4.79 | −1 | Acidic |
| | (SEQ ID NO: 362) | | | | |
| 326-340 | NAEFNEIRRVLLEEN | −0.840 | 4.49 | −2 | Acidic |
| | (SEQ ID NO: 363) | | | | |
| 361-375 | NEGVIVKVSKEHVEE | −0.560 | 4.91 | −1 | Acidic |
| | (SEQ ID NO: 364) | | | | |
| 371-385 | EHVEELTKHAKSVSK | −1.127 | 7.02 | 2 | Basic |
| | (SEQ ID NO: 365) | | | | |
| 405-419 | PDLSNNFGKLFEVKP | −0.587 | 6.49 | 0 | Neutral |
| | (SEQ ID NO: 366) | | | | |
| 416-430 | EVKPDKKNPQLQDLD | −1.840 | 4.68 | −1 | Acidic |
| | (SEQ ID NO: 367) | | | | |
| 425-439 | QLQDLDMMLTCVEIK | 0.287 | 4.03 | −2 | Acidic |
| | (SEQ ID NO: 368) | | | | |
| 431-445 | MMLTCVEIKEGALML | 1.207 | 4.53 | −1 | Acidic |
| | (SEQ ID NO: 369) | | | | |
| 441-455 | GALMLPHFNSKAMVI | 0.873 | 8.76 | 2 | Basic |
| | (SEQ ID NO: 370) | | | | |
| 451-460 | KAMVIVVVNKGTGNL | 0.833 | 10.00 | 2 | Basic |
| | (SEQ ID NO: 371) | | | | |
| 466-480 | ELVAVRKEQQQRGRR | −1.720 | 11.54 | 3 | Basic |
| | (SEQ ID NO: 372) | | | | |
| 491-505 | EGSNREVRRYTARLK | −1.720 | 10.83 | 3 | Basic |
| | (SEQ ID NO: 373) | | | | |
| 496-510 | EVRRYTARLKEGDVF | −0.900 | 8.69 | 1 | Basic |
| | (SEQ ID NO: 374) | | | | |
| 506-520 | EGDVFIMPAAHPVAI | 0.913 | 4.35 | −1 | Acidic |
| | (SEQ ID NO: 375) | | | | |
| 516-530 | HPVAINASSELHLLG | 0.447 | 5.98 | 1 | Basic |
| | (SEQ ID NO: 376) | | | | |
| 531-545 | FGINAENNHRIFLAG | −0.033 | 6.75 | 1 | Basic |
| | (SEQ ID NO: 377) | | | | |
| 536-550 | ENNHRIFLAGDKDNV | −1.060 | 5.38 | 0 | Neutral |
| | (SEQ ID NO: 378) | | | | |
| 551-565 | IDQIEKQAKDLAFPG | −0.540 | 4.56 | −1 | Acidic |
| | (SEQ ID NO: 379) | | | | |
| 569-583 | QVEKLIKNQKESHFV | −0.913 | 8.51 | 2 | Basic |
| | (SEQ ID NO: 380) | | | | |
| 581-595 | HFVSARPQSQSQSPS | −1.107 | 9.76 | 2 | Basic |
| | (SEQ ID NO: 381) | | | | |
| 612-626 | QGGKGPLLSILKAFN | 0.140 | 10.00 | 2 | Basic |
| | (SEQ ID NO: 382) | | | | |

Accordingly, in certain embodiments the epitopes to be introduced to tolerogenic nanoparticles described herein comprises one or more of the epitopes shown in Table 9, either in the form of peptides or as mRNA that encode for these peptide sequences. In certain embodiments the epitopes comprising the tolerogenic nanoparticles described herein comprise one or more of RCTKLEYDPRCVYDP (SEQ ID NO:348), SYLQGFSRNTLEAAF (SEQ ID NO:361), PDLSNNFGKLFEVKP (SEQ ID NO: 366), and/or ENNHRIFLAGDKDNV (SEQ ID NO: 378), and/or reverse translated nucleic acid sequences that encode one or more of RCTKLEYDPRCVYDP (SEQ ID NO:348), SYLQGFSRNTLEAAF (SEQ ID NO:361), PDLSNNFGKLFEVKP (SEQ ID NO: 366), and/or ENNHRIFLAGDKDNV (SEQ ID NO: 378).

IEDB-based predictions to identify potentially useful peptide sequences for therapeutic intervention in peanut allergy in humans have been published for dominant peanut allergens, including Ara h1 and h2 (see, e.g., Prickett et al. (2011) supra.: Pascal, et al; (2012) supra.). This includes prediction making of lead peptide sequences with high binding affinity for HLA-DR, -DP and -DQ to achieve widespread population coverage, as well as avoiding IgE binding sequences that could negatively impact therapeutic safety (Pricket et al. (2015) Clin. & Exp. Allergy, 45:1015-1026). The initial proof-of-principle of the translational use potential of allergen T-cell epitopes was demonstrated by immunotherapy experiments in animals sensitized to house dust mite and cat allergens (Dorofeeva et al. (2021) Allergy, 76 (1): 131-149). Noteworthy, these studies demonstrated that animal vaccination with a single dominant T-cell epitope can introduce specific non-responsiveness to repetitive intradermal injections of the same peptide, as well as the complete allergen extract. The robustness of the T-cell epitope approach has also been validated in other allergen models, evolving to the use of short peptide mixtures for intradermal administration (Worm et al. (2011) J. Allergy Clin. Immunol. 127 (1): 89-97). Currently, a phase 1 clinical trial is ongoing in Australia to assess the safety and tolerability of PVX108 (Aravax), an intradermal vaccine delivering a mixture of Ara h 1 and h2 epitope sequences (Australian New Zealand Clinical Trials Registry ACTRN 12617000692336). All considered, the central role of regulatory T-cells in orchestrating the immune response to food allergens, provides strong support for the use of the tolerogenic platform to treat peanut allergy in humans through peanut allergen epitope delivery. Our studies in mice show that a single (Ara h2) epitope is capable of providing linked suppression to a crude peanut protein extract.

Thus, in addition to the collection of human epitope sequences for peanut allergens (Ara h 1, 2, 3 and 6) described above (Tables 2-5, supra.), we have derived a short list of Ara h 1 and 2 sequences that can be regarded as lead epitopes for delivery by tolerogenic nanoparticles in humans (Table 10). These selections are based on the use of T cell proliferation data from peanut allergic subjects, MHC-II binding predictions by the IEDB, other in silico methods, as well as p1 and GRAVY data that predict a high loading capacity in PLGA nanoparticles.

TABLE 10
Illustrative extensively characterized Ara-H1 and Ara-H2 peptide sequence for
therapeutic use in humans.

| Protein | # of AA | Location | Epitope Sequence | pI | GRAVY |
|---|---|---|---|---|---|
| Ara h1 | 10 | 206-215 | FQNLQNHRIV | 9.76 | −0.640 |
| | | | (SEQ ID NO: 383) | | |
| Ara h1 | 13 | 213-225 | RIVQIEAKPNTLV | 8.75 | 0.123 |
| | | | (SEQ ID NO: 384) | | |
| Ara h1 | 20 | 206-225 | FQNLQNHRIVQIEAKPN | 8.75 | −0.440 |
| | | | TLV | | |
| | | | (SEQ ID NO: 385) | | |
| Ara h1 | 19 | 353-371 | WSTRSSENNEGVIVKVS | 6.23 | −0.942 |
| | | | KE | | |
| | | | (SEQ ID NO: 386) | | |
| Ara h1 | 19 | 409-427 | NNFGKLFEVKPDKKNPQ | 9.53 | −1.384 |
| | | | LQ | | |
| | | | (SEQ ID NO: 387) | | |
| Ara h1 | 17 | 436-452 | VEIKEGALMLPHFNSKA | 6.73 | 0.018 |
| | | | (SEQ ID NO: 388) | | |
| Ara h1 | 17 | 442-458 | ALMLPHFNSKAMVIVV | 8.80 | 1.535 |
| | | | V | | |
| | | | (SEQ ID NO: 389) | | |
| Ara h1 | 20 | 451-470 | KAMVIVVVNKGTGNLE | 8.59 | 1.150 |
| | | | LVAV | | |
| | | | (SEQ ID NO: 390) | | |
| Ara h1 | 18 | 507-524 | GDVFIMPAAHPVAINASS | 5.08 | 0.772 |
| | | | (SEQ ID NO: 391) | | |
| Ara h2 | 12 | 9-20 | ALFLLAAHASAR | 9.80 | 1.125 |
| | | | (SEQ ID NO: 392) | | |
| Ara h2 | 16 | 32-47 | SQLERANLRPCEQHLM | 6.47 | −0.906 |
| | | | (SEQ ID NO: 393) | | |
| Ara h2 | 17 | 91-107 | ELNEFENNQRCMCEALQ | 4.09 | −0.994 |
| | | | (SEQ ID NO: 394) | | |
| Ara h2 | 17 | 101-117 | RCMCEALQQIMENQSDR | 4.68 | −0.906 |
| | | | (SEQ ID NO: 395) | | |
| Ara h2 | 14 | 128-141 | RELRNLPQQCGLRA | 10.26 | −0.986 |
| | | | (SEQ ID NO: 396) | | |
| Ara h2 | 13 | 126-138 | QFKRELRNLPQQC | 9.50 | −1.469 |
| | | | (SEQ ID NO: 397) | | |

Accordingly, in certain embodiments the epitopes comprising the tolerogenic nanoparticles described herein comprises one or more of the epitopes shown in Table 10, and/or the nucleic acid(s) comprising the tolerogenic nanoparticles described herein encode one or more of the epitopes shown in Table 10.

Further refinement of above peptide selections, as well as addition of Ara-h3 and Ara-h6 epitopes can be accomplished by:

1) The use of long overlapping peptide sequences to select core T-cell epitopes that cover Ara-h3 and Ara-h6. In this regard, we note that the IEDB contains 24 epitopes for Ara h 3 that have been identified with little additional prediction making. Similarly, we have performed our own prediction for ˜70 Ara h6 sequences, which lacks additional data for therapeutic refinement. Prediction making for MHC-II binding will use the Class II HLA-peptide Binding Assay to obtain data about binding affinity for individual peptides. Unlike the traditional use of cellular assays, which depends on time-consuming and expensive patient samples, the use of the in vitro peptide binding assay allows prediction making by a one-step ex vivo procedure that can interrogate 26 HLA-DR, 11 DQ alleles and 19 DP alleles.

2) Ensuring safe immunotherapy by eliminating peptide sequences that can trigger IgE-mediated mast cell release. These sequences can be identified by using IgE dot-blot assays, and basophil activation testing (BAT), e.g., assessment of the percentage of CD63+basophils by flow cytometry (see, e.g., Prickett et al. (2011) J. Allergy. Clin. Immunol. 127, 608-615).

3) Assessment of the encapsulation efficiency for peptide loading into PLGA nanoparticles. Particle encapsulation is dependent on peptide solubility, charge and isoelectric point. This analysis is performed by GRAVY and p1 calculations, using the online chemistry tool (//web.expasy.org/protparam/). The epitopes with negative GRAVY values and pI values that deviate the furthest from 7.4, generally predict the best encapsulation efficiency.

4) In deciding amino acid sequences, it is important to consider that the presence of cysteine residues could render the proposed epitopes more susceptible to oxidation and disulfide bridging. This could impact particle loading, stability, and therapeutic efficacy. To avoid these problems, cysteines can be replaced with structurally conserved, less reactive serines. The cysteine-substituted peptides have been shown to stimulate comparable T-cell proliferation and cytokine production to the parent peptides.

5) Since the ultimate goal of the tolerogenic nanoparticles is to induce regulatory T-cells, it is important to consider essays demonstrating the likelihood of Treg generation in human peripheral blood mononuclear cells (PBMCs). This can be accomplished by harvesting CD4⁺ T-cells, using a MACS CD4⁺ T cell isolation kit II (Miltenyi Biotec). Typical enrichment allows retrieval purities of >90% (Blood 2012 119: e57-e66). The enriched CD4⁺ cell population can then be used for FACS sorting to obtain CD4⁺CD25^{hi}CD127^{Lo} that can be analyzed for IL-10 and TGF-β production in Elispot assays. An additional approach to identify the in vivo generation of Tregs is to perform a Treg conversion assay. Harvested CD4⁺CD251° T cells will be cocultured with an immortalized human liver sinusoidal microvascular endothelial cell line (commercially available) in the presence of recombinant human TGF-β1 (R&D Systems, Germany). Following the addition of TNPs to this co-culture, it is possible to assess the generation of FoxP3+ positive T cells in a flow cytometry procedure.

In various embodiments tolerogenic nanoparticles comprising a nucleic acid (e.g., DNA or mRNA) encoding one or more of the epitopes described above are provided and illustrative, but non-limiting methods and examples of such are provided below.

To prepare a multiple-epitope minigene, we performed reverse translation of the peptide into nucleotide sequences. This can be accomplished by several online tools, including, in the case of the sequences shown below use of the resource at//www. genecorner.ugent.be/rev_trans.html.

Tables 11 and 12 list nucleotide sequences of the illustrative chosen Ara h1 and 2 T cell epitopes

TABLE 11
Dominant T cell epitopes of Ara h 1 (core sequences underlined, nucleotide
sequences listed below)

| # of | | |
|---|---|---|
| AA | Location | Sequence |
| 20 | Ara h1 206-225 | FQNLQNHRIVQIEAKPNTLV (SEQ ID NO: 398) |
| | | Tttcagaacctgcagaaccatcgcattgtgcagattgaagcgaaaccgaacac |
| | | cctggtg (SEQ ID NO: 399) |
| 19 | Ara h1 353-371 | WSTRSSENNEGVIVKVSKE (SEQ ID NO: 400) |
| | | Tggagcacccgcagcagcgaaaacaacgaaggcgtgattgtgaaagtgagc |
| | | aaagaa (SEQ ID NO: 401) |
| 19 | Ara h1 409-427 | NNFGKLFEVKPDKKNPQLQ (SEQ ID NO: 402) |
| | | Aacaactttggcaaactgtttgaagtgaaaccggataaaaaaaacccgcagct |
| | | gcag (SEQ ID NO: 403) |
| 17 | Ara h1 436-452 | VEIKEGALMLPHFNSKA (SEQ ID NO: 404) |
| | | Gtggaaattaaagaaggcgcgctgatgctgccgcattttaacagcaaagcg |
| | | (SEQ ID NO: 405) |
| 17 | Ara h1 442-458 | ALMLPHFNSKAMVIVVV (SEQ ID NO: 406) |
| | | Gcgctgatgctgccgcattttaacagcaaagcgatggtgattgtggtggtg |
| | | (SEQ ID NO: 407) |
| 20 | Ara h1 451-470 | KAMVIVVVNKGTGNLELVAV (SEQ ID NO: 408) |
| | | Aaagcgatggtgattgtggtggtgaacaaaggcaccggcaacctggaactgg |
| | | tggcggtg (SEQ ID NO: 409) |
| 18 | Ara h1 507-524 | GDVFIMPAAHPVAINASS (SEQ ID NO: 410) |
| | | Ggcgatgtgtttattatgccggcggcgcatccggtggcgattaacgcgagcag |
| | | c (SEQ ID NO: 411) |

TABLE 12
Dominant T cell epitopes of Ara h 2 (core sequences underlined with nucleotide
sequence below)

| # of AA | Location | Sequence |
|---|---|---|
| 16 | Ara h2 32-47 | SQLERANLRPS*EQHLM (*C42S)(SEQ ID NO: 412) |
| | | Agccagctggaacgcgcgaacctgcgcccgagcgaacagcatctgatg |
| | | (SEQ ID NO: 413) |
| 17 | Ara h2 91-107 | ELNEFENNQRS*MS*EALQ (*C[101 + 103]S)(SEQ ID |
| | | NO: 414) |
| | | Gaactgaacgaatttgaaaacaaccagcgcagcatgagcgaagcgctgcag |
| | | (SEQ ID NO: 415) |
| 14 | Ara h2 128-141 | RELRNLPQQS*GLRA (*C137S)(SEQ ID NO: 416) |
| | | Cgcgaactgcgcaacctgccgcagcagageggcctgcgegcg (SEQ ID |
| | | NO: 417) |

Accordingly, in certain embodiments the epitopes comprising the tolerogenic nanoparticles described herein comprises one or more of the epitopes shown in Tables 11 and/or 12 and/or the nucleic acid(s) comprising the tolerogenic nanoparticles described herein encode one or more of the epitopes shown in Tables 11 and/or 12.

The need to provide flexible linkers for stringing the epitope sequences together was met by using one of the most frequently used linker peptide sequences for this purpose. This linker is comprised of stretches of Gly and Ser residues (“GS” linker), i.e., (Gly-Gly-Gly-Gly-Ser), (SEQ ID NO:418). We used a copy number of n=3 to insert three GGGGS linkers between the epitope sequences. After reverse translation into the corresponding cDNA sequence, this reads: ggcggcggcggcagcggcggcggcggcagcggcggcgg cggcagc (SEQ ID NO:419).

Ten human Ara h 1 and 2 T cell epitopes were reversed translated into cDNA coding sequences. Table 13 shows an illustrative, but non-limiting cDNA sequence encoding for 10 human Arah1 and h2 epitopes, separated by flexible linkers. The epitope sequences (in blue) were linked by (GGGGS (SEQ ID NO:420)) triplets which are reverse transcribed (in yellow).

TABLE 13
Illustrative cDNA sequence encoding for the 10
human Arah1 and h2 epitopes separated by flexible
linkers. (936 nucleotides).

Tttcagaacctgcagaaccatcgcattgtgcagattgaagcgaaaccgaa
caccctggtgggcggcggcggcagcggcggcggcggcagcggcggcggcg
gcagctggagcacccgcagcagcgaaaacaacgaaggcgtgattgtgaaa
gtgagcaaagaaggcggcggcggcagcggcggcggcggcagcggcggcgg
cggcagcaacaactttggcaaactgtttgaagtgaaaccggataaaaaaa
acccgcagctgcagggcggcggcggcagcggcggcggcagcggcggcggc
ggcagcgtggaaattaaagaaggcgcgctgatgctgccgcattttaacag
caaagcgggcggcggcggcagcggcggcggcggcageggcggcggcggca
gcgcgctgatgctgccgcattttaacagcaaagcgatggtgattgtggtg
gtgggcggcggcggcagcggcggcggcggcagcggcggcggcggcagcaa
agcgatggtgattgtggtggtgaacaaaggcaccggcaacctggaactgg
tggcggtgggcggcggcggcagcggcggcgggcggcagcggcggcggcgg
cagcggcgatgtgtttattatgccggcggcgcatccggtggcgattaacg
cgagcagcggcggcggcggcagcggcggcggcggcageggcggcggcggc
agcagccagctggaacgcgcgaacctgcgcccgagcgaacagcatctgat
gggcggcggcggcagcggcggcggcggcagcggcggcggcggcagcgaac
tgaacgaatttgaaaacaaccagcgcagcatgagcgaagcgctgcagggc
ggcggcggcagcggcggcggcggcagcggcggcggcggcagccgcgaatg
cgcaacctgccgcagcagagcggcctgcgcgcg
(SEQ ID NO: 421)

To allow epitope presentation, it is desirable for the epitopes to be proteolytically cleaved in the MHC-II endosomal compartment. LSECs express the proprotein convertase, furin, in their trans-golgi network secretory pathway (see, e.g., Evdokimov et al. (2013) Biochem. Biophys. Res. Comm. 434:22-27). It has been shown that the protein, LEDA-1/PIANP, can be proteolytically cleaved at aa7 5-79 in humans and aa 69-73 in murine LSECs, allowing the released fragments to be expressed on the surface membrane. Additional furin substrates include blood clotting factors, serum proteins and growth factor receptors such as the insulin-like growth factor receptor. Furin prefers an Arg-X-Lys/Arg-ArgV (SEQ ID NO:422) consensus sequence. The use of valine amino acid ipo X, provides the sequence, RVRR (SEQ ID NO:423), which we inserted in the cDNA line-up as the sequence: cgcgtgcgccgc (SEQ ID NO:424). This sequence was then included in the flexible linker that spaces the epitopes apart (Table 14).

TABLE 14
Illustrative but non-limiting linker comprising a furin cleavage site.

| | SEQ |
|---|---|
| | ID |
| Linker | NO |
| ggc ggc ggc ggc agc cgc gtg cgc cgc ggc ggc ggc ggc agc | 425 |
| G G G G S R V R R ↓ G G G G S | 426 |
| Furin cleavage site↑ | |

Ten human Ara h 1 and 2 T cell epitope cDNA coding sequences were linked by spacer sequences that include an inserted furin cleavable site (RVRR (SEQ ID NO:427)). This cleavage site has the reverse translated sequence: cgegtgegccgc (SEQ ID NO:428) (see, e.g., Table 15).

TABLE 15
Insertion of a furin cleavage site into the linker site of the multi-epitope Ara h
nucleic acid sequence in Table 13 yields.

TttcagaacctgcagaaccatcgcattgtgcagattgaagcgaaaccgaacaccctggtgGgcggcggcggcagccg
cgtgcgccgcggcggcggcggcagcTggagcacccgcagcagcgaaaacaacgaaggcgtgattgtgaaagtgagc
aaagaaGgcggcggcggcagccgcgtgcgccgcggcggcggcggcagcAacaactttggcaaactgtttgaagtgaaa
ccggataaaaaaaacccgcagctgcagGgcggcggcggcagccgcgtgcgccgcggcggcggcggcagcGtggaaatt
aaagaaggcgcgctgatgctgccgcattttaacagcaaagcgGgcggcggcggcagccgcgtgcgccgcggcggcggcg
gcagcGcgctgatgctgccgcattttaacagcaaagcgatggtgattgtggtggtgGgcggcggcggcagccgcgtgcgcc
gcggcggcggcggcagcAaagcgatggtgattgtggtggtgaacaaaggcaccggcaacctggaactggtggcggtgGg
cggcggcggcagccgcgtgcgccgcggcggcggcggcagcGgcgatgtgtttattatgccggcggcgcatccggtggcg
attaacgcgagcagcGgcggcggcggcagccgcgtgcgccgcggcggcggcggcagcAgccagctggaacgcgcgaa
cctgcgcccgagcgaacagcatctgatgGgcggcggcggcagccgcgtgcgccgcggcggcggcggcagcGaactgaa
cgaatttgaaaacaaccagcgcagcatgagcgaagcgctgcagGgcggcggcggcagccgcgtgcgccgcggcggcgg
cggcagccgcgaactgcgcaacctgccgcagcagagcggcctgcgcgcgcgcgtgcgccgc (SEQ ID
NO: 429)

In addition to furin, the cysteine proteases, cathepsin L (CL) and cathepsin S (CS), are known to play active roles in proteolytic cleavage in the MHC-II endosomal compartment. Antigen cleavage by these proteases release peptides, capable of completing with the degraded invariant (li) chain for MHC class II epitope presentation. Cathepsin S recognizes the peptide sequence PMGLP (SEQ ID NO:430), which is cleaved between Glycine and Lysine. Reverse translation of the cleavage site yields a cegatgggcctgccg (SEQ ID NO: 431) sequence. Cathepsin L recognizes the peptide sequence HKFR, which upon reverse translation yield a cataaatttege (SEQ ID NO:432) sequence. It would be straightforward to swap out the currently designed furin cleavage site with the alternative cathepsin recognition sites.

Endogenous CD4 epitopes can be directed from the cytosol to the MHC-II loading endosomal compartment using a TfR1-118 targeting sequence (Diebold et al. (2001) Gene Therapy, 8:487-493) (see, e.g., Table 16). To do so, we designed a novel fusion protein construct, adding TfR1-118 upstream of the string of nucleotide sequences that encode for Ara h epitopes (15). The membrane-anchoring domain of the translated fusion protein allows the linked Ara h 1 and 2 epitopes to be transported to the LSEC surface, from where it can be endocytosed to the MHC class II compartment for further proteolytic processing. An alternative approach would be to use the invariant li chain for peptide transport into the MHC-II endosomal compartment. This allows direct targeting of li/Ara h peptide fusions to the MHC II processing pathway.

TABLE 16
Human transferrin receptor protein 1 amino acid sequence (aa 1-118 are bolded).

| | SEQ ID |
|---|---|
| Sequence | NO |
| Human Transferrin receptor protein 1 | 433 |
| MMDQARSAFSNLFGGEPLSYTRFSLARQVDGDNSHVEMKLAVDEEE | |
| NADNNTKANVTKPKRCSGSICYGTIAVIVFFLIGFMIGYLGYCKGVE | |
| PKTECERLAGTESPVREEPGEDFPAARRLYWDDLKRKLSEKLDSTDFT | |
| GTIKLLNENSYVPREAGSQKDENLALYVENQFREFKLSKVWRDQHFVKI | |
| QVKDSAQNSVIIVDKNGRLVYLVENPGGYVAYSKAATVTGKLVHANFG | |
| TKKDFEDLYTPVNGSIVIVRAGKITFAEKVANAESLNAIGVLIYMDQTKF | |
| PIVNAELSFFGHAHLGTGDPYTPGFPSFNHTQFPPSRSSGLPNIPVQTISRA | |
| AAEKLFGNMEGDCPSDWKTDSTCRMVTSESKNVKLTVSNVLKEIKILNI | |
| FGVIKGFVEPDHYVVVGAQRDAWGPGAAKSGVGTALLLKLAQMFSDM | |
| VLKDGFQPSRSIIFASWSAGDFGSVGATEWLEGYLSSLHLKAFTYINLDK | |
| AVLGTSNFKVSASPLLYTLIEKTMQNVKHPVTGQFLYQDSNWASKVEK | |
| LTLDNAAFPFLAYSGIPAVSFCFCEDTDYPYLGTTMDTYKELIERIPELNK | |
| VARAAAEVAGQFVIKLTHDVELNLDYERYNSQLLSFVRDLNQYRADIKE | |
| MGLSLQWLYSARGDFFRATSRLTTDFGNAEKTDRFVMKKLNDRVMRV | |
| EYHFLSPYVSPKESPFRHVFWGSGSHTLPALLENLKLRKQNNGAFNETLF | |
| RNQLALATWTIQGAANALSGDVWDIDNEF | |
| Aa 1-118 of Human Transferrin receptor protein 1 (TFR1 1-118) | 434 |
| MMDQARSAFSNLFGGEPLSYTRFSLARQVDGDNSHVEMKLAVDEEENA | |
| DNNTKANVTKPKRCSGSICYGTIAVIVFFLIGFMIGYLGYCKGVEPKTEC | |
| ERLAGTESPVREEPGEDFPA | |
| Reverse Translation of TFR1 1-118 | 435 |
| Atgatggatcaggcgcgcagcgcgtttagcaacctgtttggcggcgaaccgctgagctatacccgctttagcctgg | |
| cgcgccaggtggatggcgataacagccatgtggaaatgaaactggcggtggatgaagaagaaaacgcggataa | |
| caacaccaaagcgaacgtgaccaaaccgaaacgctgcagcggcagcatttgctatggcaccattgcggtgattgt | |
| gttttttctgattggctttatgattggctatctgggctattgcaaaggcgtggaaccgaaaaccgaatgcgaacgcctg | |
| gcgggcaccgaaagcccggtgcgcgaagaaccgggcgaagattttccggcg | |

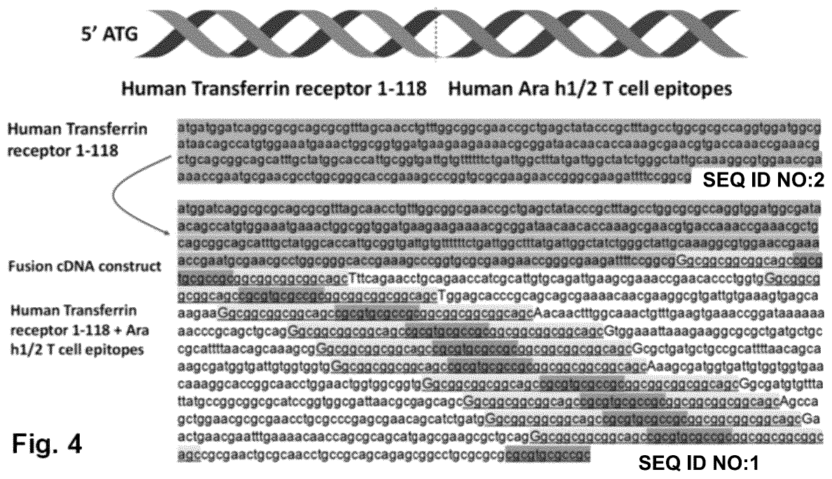

As illustrated in FIG. 4, the cDNA sequence of the transferrin receptor 1 (HFR1-118) (cyan) has been spliced upstream of the Ara h epitopes, separated by flexible, cleavable linkers. This allows the cytosol-translated fusion protein construct to be transported to the APC surface membrane, from where it could access to the MHC II compartment. Instead of the human sequence, the murine transferrin receptor membrane sequence will be used in animal studies.

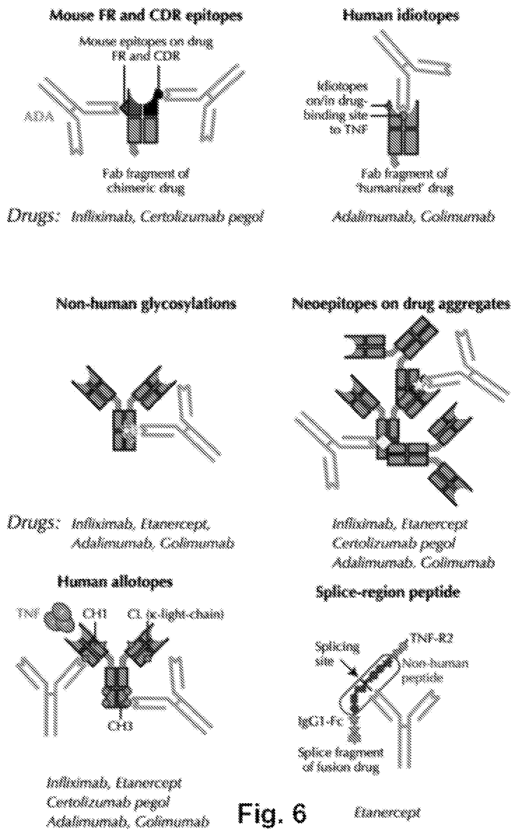

Start and stop codons are necessary for cytosolic expression of the nucleotide construct (FIG. 6). The start codon marks the site at which translation into protein sequence begins, and the stop codon marks the site at which translation ends. We use an AUG sequence (ATG in the corresponding DNA sequence) as a start codon for (methionine) together with UAA, UAG, or UGA stop codons. These codons encode for a release factor that causes translation to be terminated. The choice of the specific stop codon is determined by our codon optimization procedure that is discussed below.

TABLE 17
Codon optimized cDNA encoding sequence in FIG. 4.

ATGATGGACCAGGCCAGAAGCGCCTTCAGCAACCTGTTCGGCGGCGAGC
CACTGAGCTACACCAGATTCAGCCTGGCCAGACAGGTGGACGGCGATAA
TTCTCACGTGGAAATGAAACTGGCCGTCGACGAGGAAGAAAATGCCGAC
AACAATACCAAGGCCAATGTGACAAAGCCTAAGCGGTGCAGCGGCAGCA
TCTGTTACGGCACCATCGCCGTGATCGTGTTCTTTCTGATCGGCTTCAT
GATCGGCTATCTGGGCTACTGCAAGGGCGTGGAACCTAAGACCGAGTGC
GAGCGGCTGGCTGGCACAGAGAGCCCTGTGCGGGAAGAGCCCGGCGAAG
ATTTCCCAGCCGGCGGAGGCGGGTCTAGAGTGCGGAGAGGAGGCGGAGG
CAGCTTCCAGAACCTGCAAAACCACAGAATCGTGCAGATCGAGGCCAAG
CCCAACACCCTGGTCGGAGGCGGAGGCTCTAGAGTGAGACGCGGGGGAG
GCGGAAGCTGGTCCACCAGAAGCTCTGAGAACAATGAGGGCGTTATTGT
GAAAGTGTCCAAGGAAGGCGGCGGAGGCTCTAGAGTGAGAAGAGGGGGG
GGCGGCTCCAACAACTTCGGCAAGCTGTTTGAGGTGAAGCCTGATAAGA
AGAACCCCCAGCTGCAAGGAGGCGGAGGCAGCCGGGTGCGGAGGGGAGG
CGGAGGATCCGTGGAAATCAAGGAAGGCGCCCTGATGCTGCCCCACTTT
AACAGCAAAGCCGGGGGGGGCGGCAGCAGAGTGCGTCGGGGTGGCGGAG
GCAGTGCTCTGATGCTGCCTCACTTCAACAGCAAGGCCATGGTCATCGT
GGTGGTGGGGGGAGGGGGCAGCAGAGTTAGAAGAGGCGGCGGCGGCTCT
AAAGCCATGGTAATCGTGGTCGTGAACAAGGGAACAGGCAACCTCGAGC
TGGTGGCTGTGGGCGGTGGGGGCTCCCGGGTGCGGAGAGGCGGAGGCGG
CTCTGGCGACGTGTTCATCATGCCTGCCGCTCATCCTGTGGCCATCAAC
GCTAGCAGCGGGGGGGGCGGCAGCAGGGTGAGACGGGGCGGCGGGGGGA
GCAGCCAGCTGGAAAGAGCCAATCTGAGACCTAGCGAGCAGCACCTGAT
GGGGGGCGGAGGCTCCAGAGTGCGGGGGGGGGGGGCGGCAGCGAGCTGA
ACGAGTTCGAGAACAACCAGCGGAGCATGAGCGAGGCCCTGCAGGGGGG
GGGAGGCTCTCGCGTGCGGAGAGGAGGGGGGGGCAGCAGAGAGCTGAGA
AACCTGCCTCAGCAGAGCGGCCTGCGGGCCAGGGTGCGGAGATGA
(SEQ ID NO: 436)

Following the design of an integrated cDNA construct, codon optimization is performed. Codon optimization is a gene engineering approach for improving gene expression by changing synonymous codons based on an organism's codon bias. For the purpose of gene design, we used an accessible and user-friendly codon optimization tool from GenSmart™ Codon Optimization (//www.genscript.com/gensmart-free-gene-codon-optimization.html). This tool uses a novel algorithm based on the “Population Immune Algorithm”, which takes advantage of population genetics as well as immunology theory. The paradigm takes into consideration more than 200 parameters required for optimal gene expression, including GC content, codon usage and content index, RNase splicing sites, as well as screening for cis-acting destabilizing motifs. This allows a multifactor approach to ensure that all the key variables in our target gene design carry the appropriate weight. Consequently, this allows the customized gene sequences to be optimally designed for the expression of functional and active protein and peptide sequences. Since all of the parameters are integrated into the algorithm, no additional user input is required other than adding the gene sequence and the desired species. Moreover, the optimized sequences can be ordered with ease by the GenSmart™ Instant Quote System, including archiving for future use. Upon entry of the codon sequence displayed in FIG. 4, the optimized codon construct shown in Table 17 yielded a GC content of 63.48%, 17.

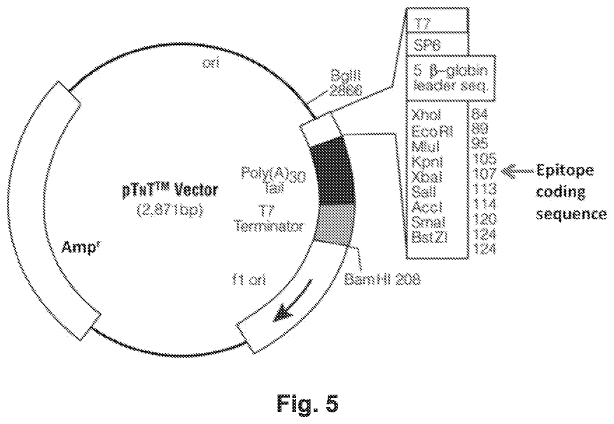

In one illustrative, but non-limiting embodiment, we use plasmid vectors that employee a viral RNA polymerase (typically T7 or SP6), as well as incorporating sterilizing elements for expressing our mRNA transcripts (FIG. 5). These plasmid elements include untranslated sequences upstream (5′UTR) and downstream (3′ UTR) of the spliced-in cDNA sequence of interest. The 3′ UTR includes a synthetic poly(A) tail, which is important for efficient protein transcription. Longer poly(A) tails usually result in increased mRNA half-life and more efficient protein expression. Among the many commercially available plasmid vectors for mRNA preparation, most of our experience has been with pTNT from Promega or CleanCap from TriLink. As an illustrative example, we show the sequence construct for the pTNT plasmid from Promega (FIG. 5). This vector contains tandem T7 and SP6 promoters, a 5′ UTR from rabbit β-globin, and a synthetic poly(A) 30 tail.

Our plasmids are amplified in Escherichia coli and purified with a commercially available plasmid preparation kit that yields high quality, endotoxin-free plasmid DNA. Subsequently, the plasmid DNA is linearized to avoid circular transcription, yielding RNA strands of appropriate size. The linearized plasmids are generated in large batches that are stored at −20° C.

Our linearized DNA plasmids are transcribed to RNA using T7 or SP6 RNA polymerases. Milligram quantities of RNA are produced, using optimized buffers and high concentrations of rNTPs and inorganic pyrophosphatase (to prevent the inhibitory effects of pyrophosphate released during ribonucleoside triphosphate incorporation). All the necessary components are available in kits from Promega (RiboMAX™ Large Scale RNA Production Systems) or other commercial sources such as TriLink. A 1 mL reaction results in approximately 2-5 mg of mRNA.

Proper mRNA capping facilitates the production of biologically active and non-immunogenic mRNA. We use a 7-methyl-guanosine cap structure to accomplish mRNA stability and translational efficacy. Cap 0 structures (m7G(5′)ppp(5′)NpN) are added to the 5′ end of RNA using a virus capping enzyme with a capping efficacy close to 100%. The kit that we use, ScriptCap m7G Capping Kit from Cellscript, includes a vaccinia virus capping enzyme. This kit contains all the enzymatic analogues required for cap construction, i.e., a mRNA triphosphatase, guanylyltransferase, and guanine-7-methyltransferase. We also have available another reagent for co-transcriptional 5′ capping, namely CleanCap® from TriLink, which generates a natural Cap 1 structure that evades innate immune activation in vivo.

We perform quality control of our capped mRNA through the performance of denaturing agarose gel electrophoresis, in vitro transfection, and western blotting. We look for transcripts that appear as a single band of the expected size. Longer transcripts point to insufficient plasmid linearization or the presence of cryptic antisense promoters. Smaller transcripts can be the result of RNA secondary structures such as poly-T regions or repeats.

In vitro transfection and western blotting analysis is performed to assess translation efficacy of the RNA by performance of transfection studies in cultured human cell lines, including LSECs. Effective translation is dependent on the integrity and secondary RNA structure, including presence of a cap 1 structure. Codon usage may also affect translation efficacy.

The foregoing methods and constructs are illustrative and non-limiting. Using the teaching provided herein, numerous nucleic acid and peptide single- or multiple-epitope constructs for inclusion in tolerogenic nanoparticles described herein are available to one of skill in the art. In this respect, it is noted that Example 4 describes the design of coding sequences for the expression of Ara h 2 and 6 epitopes in preclinical animal studies. Example 5 shows synthesis, biodistribution and animal data for use of a mRNA encoding for multiple copies of peanut epitope 4, showing efficacy in liver targeting, Treg generation and a partial response in an animal anaphylaxis model.

### House Dust Mite Allergy

In addition to tolerogenic therapy for food allergens, LSEC-targeting nanoparticles can also be used to deliver peptides and nucleic acids that encode for inhalable allergens. The house dust mite (HDM) is one of the most important producers of IgE-associated allergies worldwide. HDM-allergic patients suffer from severe and chronic respiratory symptoms such as rhinitis and asthma as well as from atopic dermatitis (Huan et al. (2019) Allergy, 74:2461-2478). Major dust mite allergens are derived from the genus Dermatophagoides. Dermatophagoides pteronyssinus (Der p) and D. farinae (Der f), which may shed more than 30 different allergenic molecules. To date, 17 groups of allergens from Dermatophogoides sp. have been described (see, e.g., www.allergen.org), particularly the group 1 and 2 allergens from Dermatophagoides pteronyssinus (Der p) and D. farinaE (Der f). These allergens, which are secreted from mite bodies or excreted in their feces, are among the strongest immunogens in humans. Der p 1 and Der f 1 are proteins with cysteine protease activity while Der p 2 and Der f 2 contribute to allergenicity by interacting with the innate immune system (Hinz et al. (2015) Clin. Exp. Allergy. 45:1601-1612). Other HDM allergens, Der p 4, p 5, p 7, p 21, p 23, also rank among the most common mite allergens. Among those, Der p 1, 2, 5, 7, 21, and 23 are most relevant towards contributing IgE binding HDM epitopes (Huan et al. (2019) Allergy, 74:2461-2478).

The dust mite has been selected as an example of an inhalable allergen that can be used for the development of multi-epitope peptide and mRNA vaccines, hypothesized to be capable of inducing regulatory T-cells. The same principle can also be applied to other categories of inhalable allergens, including tree/grass/weed pollen, mold spores and fungi (indoor and outdoor species), animal dander (skin and saliva from cats, dogs, mice, laboratory animals), cockroach particles. Our approach is to use synthetic peptides or reverse translated nucleotide sequences that encode for small non-IgE binding epitopes of less than 20 amino acids, including allowance for MHC-II presentation.

### IEDB Based Predictions

The selection of human T-cell epitopes take into consideration affinity binding to HLA class II molecules, which are encoded by one of three highly polymorphic loci, HLA-DR, HLA-DP, or HLA-DQ. In the following tables, we list epitope predictions for major HDM proteins (Der p1, p2, p5, p7, p23), which could be retrieved from the IEDB database (https://www.iedb.org/result_v3.php?cookie_id=b3b55e). These peptide selections were further refined by identifying immunodominant core sequences that are capable of inducing proliferative responses and cytokine production (e.g., IFN-γ, IL-5, IL-4, IL-13) in human peripheral blood mononuclear cells (PBMC), e.g., by using CFSE labeling, ELISA/Elispot assays or tetramer binding. Further selection for delivery of peptide epitopes will consider peptide solubility characteristics and the ability to induce regulatory T cells as described for peanut allergens. Tables 18-23 demonstrate a series of peptide predictions for different Der p allergens, from which selections are made for developing multi-epitope vaccines.

### Der p 1 Predictions:

Der p 1 is located in the mid-gut and fecal pellets of the European house dust mite Dermatophagoides pteronyssinus. Der p 1 is produced in the gastrointestinal tract, where it is responsible for zymogen activation, including for the serine proteases Der p 3, Der p 6, and Der p 9. These dust mite products are secreted as potent allergens.

Der p 1 is a major source of HDM allergy, capable of triggering most IgE responses in combination with the group 2 allergen, Der p 2. Because of the prominence of Der p 1 in HDM allergy, this allergen is included in all HDM vaccines. Der p 1 is a major component of crude dust mite extracts and is also used in the form of hypoallergenic derivatives that have been used for immunotherapy. Dampening of the response to Der p 1 frequently suffice for alleviating clinical allergy symptoms.

TABLE 18
Deposited IEDB human Der p 1 epitopes, confirmed by T cell testing (Huang et al.
(2019) Allergy. 74: 2461; Oseroff et al. (2012) J. Immunol. 189: 1800; Wambre et al. (2011)
Clin. Exp. Allergy, 41: 192)

| Epitope | | Starting | Ending | Antigen |
|---|---|---|---|---|
| source | Epitope Sequence | Position | Position | Name |
| www.iedb.org/ | DLMMIEEYPYVVIL | 289 | 302 | Der p1 |
| epitope/9210 | (SEQ ID NO: 437) | | | allergen |
| | | | | precursor |
| www.iedb.org/ | DLRQMRTVTPIRMQGGCGS | 95 | 113 | Der p 1 |
| epitope/9265 | (SEQ ID NO: 438) | | | allergen |
| | | | | precursor |
| www.iedb.org/ | FRHYDGRTIIQRDNGYQPN | 230 | 248 | Der p 1 |
| epitope/17595 | (SEQ ID NO: 439) | | | allergen |
| | | | | precursor |
| www.iedb.org/ | GCGSCWAFSGVAATESAYL | 110 | 128 | Der p 1 |
| epitope/18868 | (SEQ ID NO: 440) | | | allergen |
| | | | | precursor |
| www.iedb.org/ | HNGVVQESYYRYVAREQSC | 165 | 183 | Der p 1 |
| epitope/24376 | (SEQ ID NO: 441) | | | allergen |
| | | | | precursor |
| www.iedb.org/ | IEYIQHNGVVQESYYRYVA | 160 | 178 | Der p 1 |
| epitope/26021 | (SEQ ID NO: 442) | | | allergen |
| | | | | precursor |
| www.iedb.org/ | IRMQGGCGSCWAFSGVAAT | 105 | 123 | Der p 1 |
| epitope/28341 | (SEQ ID NO: 443) | | | allergen |
| | | | | precursor |
| www.iedb.org/ | KNRFLMSAEAFEHLKTQFDL | 58 | 94 | Der p 1 |
| epitope/32601 | NAETNACSINGNAPAEI | | | allergen |
| | (SEQ ID NO: 444) | | | precursor |
| www.iedb.org/ | NAQRFGISNYCQI | 187 | 199 | Der p 1 |
| epitope/43289 | (SEQ ID NO: 445) | | | allergen |
| | | | | precursor |
| www.iedb.org/ | NKIREALAQTHSAIAVIIG | 205 | 223 | Der p 1 |
| epitope/44510 | (SEQ ID NO: 446) | | | allergen |
| | | | | precursor |
| www.iedb.org/ | QESYYRYVAREQSCRRPNA | 170 | 188 | Der p 1 |
| epitope/50703 | (SEQ ID NO: 447) | | | allergen |
| | | | | precursor |
| www.iedb.org/ | QIYPPNANKIREALAQTHSAI | | | Der P 1 |
| epitope/51158 | AHYWT | | | |
| | (SEQ ID NO: 448) | | | |
| www.iedb.org/ | QSCRRPNAQRFGISNYCQI | 181 | 199 | Der p 1 |
| epitope/52300 | (SEQ ID NO: 449) | | | allergen |
| | | | | precursor |
| www.iedb.org/ | RFGISNYCQIY | 190 | 200 | Der p 1 |
| epitope/53705 | (SEQ ID NO: 450) | | | allergen |
| | | | | precursor |
| www.iedb.org/ | RFGISNYCQIYPPNANKIREA | 190 | 211 | Der p 1 |
| epitope/53706 | L | | | allergen |
| | (SEQ ID NO: 451) | | | precursor |
| www.iedb.org/ | RFGISNYCQIYPPNVNKIR | 208 | 226 | Peptidase 1 |
| epitope/53707 | (SEQ ID NO: 452) | | | precursor |
| www.iedb.org/ | RPNAQRFGISNYCQIYPPN | 185 | 203 | Der p 1 |
| epitope/55237 | (SEQ ID NO: 453) | | | allergen |
| | | | | precursor |
| www.iedb.org/ | SAYLAHRNQSLDLAEQELVD | 63 | 85 | major house |
| epitope/57053 | CAS | | | dust allergen |
| | (SEQ ID NO: 454) | | | |
| www.iedb.org/ | SAYLAYRNQSLDLAEQELV | 125 | 143 | Der p 1 |
| epitope/57055 | (SEQ ID NO: 455) | | | allergen |
| | | | | precursor |
| www.iedb.org/ | TIPRGIEYIQHNGVVQESY | 155 | 173 | Der p 1 |
| epitope/64424 | (SEQ ID NO: 456) | | | allergen |
| | | | | precursor |
| www.iedb.org/ | TNACSINGNAPAEIDLRQ | 81 | 98 | Der p 1 |
| epitope/65284 | (SEQ ID NO: 457) | | | allergen |
| | | | | precursor |
| www.iedb.org/ | VIIGIKDLDAFRHYDGRTI | 220 | 238 | Der p 1 |
| epitope/68988 | (SEQ ID NO: 458) | | | allergen |
| | | | | precursor |
| www.iedb.org/ | VRNSWDTNWGDNGYGYFA | 268 | 302 | Der p 1 |
| epitope/70793 | ANIDLMMIEEYPYVVIL | | | allergen |
| | (SEQ ID NO: 459) | | | precursor |
| www.iedb.org/ | YAYVAREQSCR | 174 | 185 | Der P 1 |
| epitope/73482 | (SEQ ID NO: 460) | | | |
| www.iedb.org/ | YPPNVNKIREALAQTHSAI | 218 | 236 | Peptidase 1 |
| epitope/75381 | (SEQ ID NO: 461) | | | precursor |
| www.iedb.org/ | YQPNYHAVNIVGYSNAQGV | 245 | 263 | Der p 1 |
| epitope/75531 | (SEQ ID NO: 462) | | | allergen |
| | | | | precursor |
| www.iedb.org/ | VQESYYRYVAREQSCRRPN | 169 | 197 | Der p 1 |
| epitope/78293 | AQRFGISNYC | | | allergen |
| | (SEQ ID NO: 463) | | | precursor |
| www.iedb.org/ | ALAQTHTAIAVIIGIKDLD | 211 | 229 | Der P 1 |
| epitope/80334 | (SEQ ID NO: 464) | | | |
| www.iedb.org/ | RFGISNYCQIYPPNANKIR | 190 | 208 | Der p 1 |
| epitope/87858 | (SEQ ID NO: 465) | | | allergen |
| | | | | precursor |
| www.iedb.org/ | AVNIVGYSNAQGVDY | 251 | 265 | Der p 1 |
| epitope/150151 | (SEQ ID NO: 466) | | | allergen |
| | | | | precursor |
| www.iedb.org/ | LRQMRTVTPIRMQGG | 96 | 110 | Der p 1 |
| epitope/150405 | (SEQ ID NO: 467) | | | allergen |
| | | | | precursor |
| www.iedb.org/ | RFGISNYCQIYPPNV | 208 | 222 | Peptidase 1 |
| epitope/150525 | (SEQ ID NO: 468) | | | precursor |
| www.iedb.org/ | ARKNFLESVKYVQSN | 28 | 42 | Der p 1 |
| epitope/168207 | (SEQ ID NO: 469) | | | allergen |
| | | | | precursor |
| www.iedb.org/ | DNGYGYFAANIDLMM | 278 | 292 | Der p 1 |
| epitope/168361 | (SEQ ID NO: 470) | | | allergen |
| | | | | precursor |
| www.iedb.org/ | EIDLRQMRTVTPIRM | 93 | 107 | Der p 1 |
| epitope/168446 | (SEQ ID NO: 471) | | | allergen |
| | | | | precursor |
| www.iedb.org/ | GGAINHLSDLSLDEF | 43 | 57 | Der p 1 |
| epitope/168654 | (SEQ ID NO: 472) | | | allergen |
| | | | | precursor |
| www.iedb.org/ | KAFNKSYATFEDEEA | 13 | 27 | Der p 1 |
| epitope/168935 | (SEQ ID NO: 473) | | | allergen |
| | | | | precursor |
| www.iedb.org/ | LALSAVYARPSSIKT | 11 | 25 | Peptidase 1 |
| epitope/169063 | (SEQ ID NO: 474) | | | precursor |
| www.iedb.org/ | LESVKYVQSNGGAIN | 33 | 47 | Der p 1 |
| epitope/169089 | (SEQ ID NO: 475) | | | allergen |
| | | | | precursor |
| www.iedb.org/ | QTHSAIAVIIGIKDL | 213 | 227 | Der p 1 |
| epitope/169610 | (SEQ ID NO: 476) | | | allergen |
| | | | | precursor |
| www.iedb.org/ | REALAQTHSAIAVII | 208 | 222 | Der p 1 |
| epitope/169631 | (SEQ ID NO: 477) | | | allergen |
| | | | | precursor |
| www.iedb.org/ | RGIEYIQHNGVVQES | 158 | 172 | Der p 1 |
| epitope/169636 | (SEQ ID NO: 478) | | | allergen |
| | | | | precursor |
| www.iedb.org/ | SCWAFSGVAATESAY | 113 | 127 | Der p 1 |
| epitope/169687 | (SEQ ID NO: 479) | | | allergen |
| | | | | precursor |
| www.iedb.org/ | SGVAATESAYLAYRN | 118 | 132 | Der p 1 |
| epitope/169714 | (SEQ ID NO: 480) | | | allergen |
| | | | | precursor |
| www.iedb.org/ | GRTIIQRDNGYQPNYHAVNI | 235 | 267 | Der p 1 |
| epitope/174132 | VGYSNAQGVDYWI | | | allergen |
| | (SEQ ID NO: 481) | | | |
| www.iedb.org/ | HNGVVQESYYRYVAREQSC | 165 | 195 | Der p 1 |
| epitope/174141 | RRPNAQRFGISN | | | allergen |
| | (SEQ ID NO: 482) | | | |
| www.iedb.org/ | KDLDAFRHYDGRTIIQRDNG | 225 | 255 | Der p 1 |
| epitope/174159 | YQPNYHAVNIV | | | allergen |
| | (SEQ ID NO: 483) | | | |
| www.iedb.org/ | REQSCRRPNAQRFGISNYCQI | 197 | 233 | Der p 1 |
| epitope/174258 | YPPNVNKIREALAQTH | | | allergen |
| | (SEQ ID NO: 484) | | | precursor |
| www.iedb.org/ | VGYSNAQGVDYWIVRNSWD | 255 | 288 | Der p 1 |
| epitope/174308 | TNWGDNGYGYFAANI | | | allergen |
| | (SEQ ID NO: 485) | | | |
| www.iedb.org/ | ATESAYLAYRNQSLDLAEQE | 122 | 164 | Der p 1 |
| epitope/969040 | LVDCASQHGCHGDTIPRGIE | | | allergen |
| | YIQ | | | |
| | (SEQ ID NO: 486) | | | |
| www.iedb.org/ | TNACSINGNAPAEIDLRQMR | 81 | 121 | Der p 1 |
| epitope/969080 | TVTPIRMQGGCGSCWAFSGV | | | allergen |
| | A | | | |
| | (SEQ ID NO: 487) | | | |
| www.iedb.org/ | VRNSWDTNWGDNGYGYFA | 268 | 303 | Der P 1 |
| epitope/969087 | ANIDIMMIEEYPYVVIL | | | |
| | (SEQ ID NO: 488) | | | |

Accordingly, in certain embodiments the epitopes comprising the tolerogenic nanoparticles described herein comprises one or more of the epitopes shown in Table 18 and/or the nucleic acid(s) comprising the tolerogenic nanoparticles described herein encode one or more of the epitopes shown in Table 18.

### Der p 2 Predictions:

The group 2 antigen, Der p2, is capable of generating IgE-mediated responses in over 80% of dust mite allergic individuals.

TABLE 19
Deposited IEDB human Der p 2 epitopes, confirmed by T cell testing (Huang et al.
(2019) Allergy, 74: 2461; Chen et al. (2012) Allergy, 67: 609; Crack et al. (2012) Clin. Exp.
Dermatol. 37: 266).

| | | Starting | Ending | |
|---|---|---|---|---|
| Epitope IRI | Sequence | Position | Position | Antigen Name |
| www.iedb.org/ | ASIDGLGVDVPGID | 56 | 69 | Mite group 2 |
| epitope/4469 | (SEQ ID NO: 489) | | | allergen Der p 2 |
| www.iedb.org/ | CHGSEPCIIHRGKPF | 21 | 35 | Der p 2 allergen |
| epitope/6386 | (SEQ ID NO: 490) | | | precursor |
| www.iedb.org/ | CPLVKGQQYDIKYTWNVPKIA | 78 | 104 | Der p 2 allergen |
| epitope/6810 | PKSENV | | | precursor |
| | (SEQ ID NO: 491) | | | |
| www.iedb.org/ | DIKYTWNVPKIAPKSENVVVT | 87 | 112 | Der p 2 allergen |
| epitope/8692 | VKVMG | | | precursor |
| | (SEQ ID NO: 492) | | | |
| www.iedb.org/ | DQVDVKDCANHEIKK | 1 | 15 | Der p 2 allergen |
| epitope/9915 | (SEQ ID NO: 493) | | | precursor |
| www.iedb.org/ | DQVDVKDCANHEIKKVLVPG | 1 | 20 | Der p 2 allergen |
| epitope/9916 | (SEQ ID NO: 494) | | | precursor |
| www.iedb.org/ | EAVFEANQNTKTAK | 55 | 68 | Mite group 2 |
| epitope/11212 | (SEQ ID NO: 495) | | | allergen Der p 2 |
| | | | | precursor |
| www.iedb.org/ | FEAVQNTKTAKIEIK | 37 | 55 | Mite group 2 |
| epitope/15493 | (SEQ ID NO: 496) | | | allergen Der p 2 |
| www.iedb.org/ | GCHGSEPCIIHRGK | 20 | 33 | Der p 2 allergen |
| epitope/18878 | (SEQ ID NO: 497) | | | precursor |
| www.iedb.org/ | HEIKKVLVPGCHGS | 11 | 24 | Der p 2 allergen |
| epitope/23697 | (SEQ ID NO: 498) | | | precursor |
| www.iedb.org/ | HEIKKVLVPGCHGSE | 11 | 25 | Der p 2 allergen |
| epitope/23698 | (SEQ ID NO: 499) | | | precursor |
| www.iedb.org/ | HGSEPCIIHRGKPFQLEAV | 22 | 40 | Der p 2 allergen |
| epitope/23951 | (SEQ ID NO: 500) | | | precursor |
| www.iedb.org/ | HGSEPCIIHRGKPFQLEAVFEA | 39 | 67 | Mite group 2 |
| epitope/23952 | NQNTKTA | | | allergen Der p 2 |
| | (SEQ ID NO: 501) | | | precursor |
| www.iedb.org/ | IDGLEVDVPGIDPNAC | 58 | 73 | Der p 2 allergen |
| epitope/25576 | (SEQ ID NO: 502) | | | precursor |
| www.iedb.org/ | IHRGKPFQLEAVFE | 29 | 42 | Der p 2 allergen |
| epitope/26406 | (SEQ ID NO: 503) | | | precursor |
| www.iedb.org/ | IIHRGKPFQLEAV | 28 | 40 | Der p 2 allergen |
| epitope/26527 | (SEQ ID NO: 504) | | | precursor |
| www.iedb.org/ | KGQQYDIKYTWNVPKIAPK | 82 | 100 | Der p 2 allergen |
| epitope/31049 | (SEQ ID NO: 505) | | | precursor |
| www.iedb.org/ | KIEIKASIDGLEVDV | 51 | 65 | Der p 2 allergen |
| epitope/31217 | (SEQ ID NO: 506) | | | precursor |
| www.iedb.org/ | KIEIKASIDGLEVDVPGIDPNAC | 51 | 77 | Der p 2 allergen |
| epitope/31219 | HYMK | | | precursor |
| | (SEQ ID NO: 507) | | | |
| www.iedb.org/ | KPFQLEAVFEANQNT | 50 | 64 | Mite group 2 |
| epitope/32709 | (SEQ ID NO: 508) | | | allergen Der p 2 |
| | | | | precursor |
| www.iedb.org/ | LEVDVPGIDPNACHY | 61 | 75 | Der p 2 allergen |
| epitope/35733 | (SEQ ID NO: 509) | | | precursor |
| www.iedb.org/ | LEVDVPGIDPNACHYMKCPLV | 61 | 86 | Der p 2 allergen |
| epitope/35735 | KGQQY | | | precursor |
| | (SEQ ID NO: 510) | | | |
| www.iedb.org/ | MGDDGVLACAIATHAKIRD | 128 | 146 | Mite group 2 |
| epitope/41559 | (SEQ ID NO: 511) | | | allergen Der p 2 |
| | | | | precursor |
| www.iedb.org/ | NACHYMKCPLVKGQQ | 71 | 85 | Der p 2 allergen |
| epitope/43128 | (SEQ ID NO: 512) | | | precursor |
| www.iedb.org/ | NACHYMKCPLVKGQQY | 71 | 86 | Der p 2 allergen |
| epitope/43129 | (SEQ ID NO: 513) | | | precursor |
| www.iedb.org/ | NVVVTVKVMGDDGVLA | 120 | 135 | Mite group 2 |
| epitope/46519 | (SEQ ID NO: 514) | | | allergen Der p 2 |
| | | | | precursor |
| www.iedb.org/ | RGKPFQLEAVFEANQNTKTAK | 48 | 77 | Mite group 2 |
| epitope/53899 | IEIKASIDG | | | allergen Der p 2 |
| | (SEQ ID NO: 515) | | | precursor |
| www.iedb.org/ | RGKPPQLEAVFEAVQNT | 31 | 60 | Mite group 2 |
| epitope/53902 | (SEQ ID NO: 516) | | | allergen Der p 2 |
| www.iedb.org/ | SENVVVTVKVMGDD | 118 | 131 | Mite group 2 |
| epitope/57584 | (SEQ ID NO: 517) | | | allergen Der p 2 |
| | | | | precursor |
| www.iedb.org/ | SENVVVTVKVMGDDG | 118 | 132 | Mite group 2 |
| epitope/57585 | (SEQ ID NO: 518) | | | allergen Der p 2 |
| | | | | precursor |
| www.iedb.org/ | TKTAKIEIKASIDG | 64 | 77 | Mite group 2 |
| epitope/64679 | (SEQ ID NO: 519) | | | allergen Der p 2 |
| | | | | precursor |
| www.iedb.org/ | TWNVPKIAPKSENVV | 91 | 105 | Der p 2 allergen |
| epitope/67286 | (SEQ ID NO: 520) | | | precursor |
| www.iedb.org/ | VKGQQYDIKYTWNVP | 81 | 95 | Der p 2 allergen |
| epitope/69229 | (SEQ ID NO: 521) | | | precursor |
| www.iedb.org/ | VKGQQYDIKYTWNVPK | 81 | 96 | Der p 2 allergen |
| epitope/69230 | (SEQ ID NO: 522) | | | precursor |
| www.iedb.org/ | VLACAIATHAKIRD | 116 | 129 | Der p 2 allergen |
| epitope/69355 | (SEQ ID NO: 523) | | | precursor |
| www.iedb.org/ | VLVPGCHGSEPCIIHR | 16 | 31 | Der p 2 allergen |
| epitope/69841 | (SEQ ID NO: 524) | | | precursor |
| www.iedb.org/ | VMGDDGVLACAIAT | 127 | 140 | Mite group 2 |
| epitope/69953 | (SEQ ID NO: 525) | | | allergen Der p 2 |
| | | | | precursor |
| www.iedb.org/ | VVVTVKVMGDDGVLACAIAT | 121 | 146 | Mite group 2 |
| epitope/71889 | HAKIRD | | | allergen Der p 2 |
| | (SEQ ID NO: 526) | | | precursor |
| www.iedb.org/ | WNVPKIAPKSENVV | 92 | 105 | Der p 2 allergen |
| epitope/72918 | (SEQ ID NO: 527) | | | precursor |
| www.iedb.org/ | PCIIHRGKPFQLEAV | 26 | 40 | Der p 2 allergen |
| epitope/150486 | (SEQ ID NO: 528) | | | precursor |
| www.iedb.org/ | APKSENVVVTVKVMGDNGVL | 98 | 129 | Der p 2 allergen |
| epitope/164595 | ACAIATHAKIRD | | | precursor |
| | (SEQ ID NO: 529) | | | |
| www.iedb.org/ | CHGSEPCIIHRGKPFQLEAVFE | 21 | 51 | Der p 2 allergen |
| epitope/164614 | ANQNSKTAK | | | precursor |
| | (SEQ ID NO: 530) | | | |
| www.iedb.org/ | DQVDVKDCANHEIKKVLVPGC | 1 | 33 | Der p 2 allergen |
| epitope/164636 | HGSEPCIIHRGK | | | precursor |
| | (SEQ ID NO: 531) | | | |
| www.iedb.org/ | EANQNSKTAKIEIKASIEGLEV | 41 | 73 | Mite group 2 |
| epitope/164643 | DVPGIDPNAC | | | allergen Der p 2 |
| | (SEQ ID NO: 532) | | | |
| www.iedb.org/ | EVDVPGIDPNACHYMKCPLVK | 62 | 103 | Mite group 2 |
| epitope/164658 | GQQYDIKYTWIVPKIAPKSEN | | | allergen Der p 2 |
| | (SEQ ID NO: 533) | | | |
| www.iedb.org/ | DIKYTWNVPKI | 87 | 97 | Der p 2 allergen |
| epitope/167087 | (SEQ ID NO: 534) | | | precursor |
| www.iedb.org/ | DIKYTWNVPKIA | 87 | 98 | Der p 2 allergen |
| epitope/167088 | (SEQ ID NO: 535) | | | precursor |
| www.iedb.org/ | DIKYTWNVPKIAPKSEN | 87 | 103 | Der p 2 allergen |
| epitope/167089 | (SEQ ID NO: 536) | | | precursor |
| www.iedb.org/ | IKYTWNVPKIAPKSEN | 88 | 103 | Der p 2 allergen |
| epitope/167112 | (SEQ ID NO: 537) | | | precursor |
| www.iedb.org/ | QQYDIKYTWNVPKI | 84 | 97 | Der p 2 allergen |
| epitope/167144 | (SEQ ID NO: 538) | | | precursor |
| www.iedb.org/ | QQYDIKYTWNVPKIA | 84 | 98 | Der p 2 allergen |
| epitope/167145 | (SEQ ID NO: 539) | | | precursor |
| www.iedb.org/ | QQYDIKYTWNVPKIAP | 84 | 99 | Der p 2 allergen |
| epitope/167146 | (SEQ ID NO: 540) | | | precursor |
| www.iedb.org/ | QQYDIKYTWNVPKIAPK | 84 | 100 | Der p 2 allergen |
| epitope/167147 | (SEQ ID NO: 541) | | | precursor |
| www.iedb.org/ | QQYDIKYTWNVPKIAPKS | 84 | 101 | Der p 2 allergen |
| epitope/167148 | (SEQ ID NO: 542) | | | precursor |
| www.iedb.org/ | QQYDIKYTWNVPKIAPKSE | 84 | 102 | Der p 2 allergen |
| epitope/167149 | (SEQ ID NO: 543) | | | precursor |
| www.iedb.org/ | QQYDIKYTWNVPKIAPKSEN | 84 | 103 | Der p 2 allergen |
| epitope/167150 | (SEQ ID NO: 544) | | | precursor |
| www.iedb.org/ | QYDIKYTWNVPK | 85 | 96 | Der p 2 allergen |
| epitope/167153 | (SEQ ID NO: 545) | | | precursor |
| www.iedb.org/ | QYDIKYTWNVPKI | 85 | 97 | Der p 2 allergen |
| epitope/167154 | (SEQ ID NO: 546) | | | precursor |
| www.iedb.org/ | QYDIKYTWNVPKIAPKSEN | 85 | 103 | Der p 2 allergen |
| epitope/167155 | (SEQ ID NO: 547) | | | precursor |
| www.iedb.org/ | YDIKYTWNVPK | 86 | 96 | Der p 2 allergen |
| epitope/167177 | (SEQ ID NO: 548) | | | precursor |
| www.iedb.org/ | YDIKYTWNVPKI | 86 | 97 | Der p 2 allergen |
| epitope/167178 | (SEQ ID NO: 549) | | | precursor |
| www.iedb.org/ | YDIKYTWNVPKIA | 86 | 98 | Der p 2 allergen |
| epitope/167179 | (SEQ ID NO: 550) | | | precursor |
| www.iedb.org/ | YDIKYTWNVPKIAPKSEN | 86 | 103 | Der p 2 allergen |
| epitope/167180 | (SEQ ID NO: 551) | | | precursor |

Accordingly, in certain embodiments the epitopes comprising the tolerogenic nanoparticles described herein comprises one or more of the epitopes shown in Table 19 and/or the nucleic acid(s) comprising the tolerogenic nanoparticles described herein encode one or more of the epitopes shown in Table 19.

### Der p3 Predictions:

TABLE 20
Deposited human Der p 3 epitopes in the IEDB, confirmed by T cell testing
(Oseroff et al. (2012) J. Immunol. 189: 1800-1811)

| | | Starting | Ending | Parent |
|---|---|---|---|---|
| Epitope IRI | Sequence | Position | Position | Protein |
| www.iedb.org/epitope/ | EYWILTAAHCV | 61 | 75 | Der p 3 |
| 168521 | AGQT | | | |
| | (SEQ ID NO: 552) | | | |
| www.iedb.org/epitope/ | GGEKISVAKIFA | 91 | 105 | Der p 3 |
| 168657 | HEK | | | |
| | (SEQ ID NO: 553) | | | |
| www.iedb.org/epitope/ | IDNDIALIKLKSP | 111 | 125 | Der p 3 |
| 168819 | MK | | | |
| | (SEQ ID NO: 554) | | | |
| www.iedb.org/epitope/ | IRYNSLKHSLGG | 81 | 95 | Der p 3 |
| 168900 | EKI | | | |
| | (SEQ ID NO: 555) | | | |
| www.iedb.org/epitope/ | SELRRVDIAVVS | 166 | 180 | Der p 3 |
| 169697 | RKE | | | |
| | (SEQ ID NO: 556) | | | |
| www.iedb.org/epitope/ | TLANPILPASPN | 16 | 30 | Der p 3 |
| 169855 | ATI | | | |
| | (SEQ ID NO: 557) | | | |

Accordingly, in certain embodiments the epitopes comprising the tolerogenic nanoparticles described herein comprises one or more of the epitopes shown in Table 20 and/or the nucleic acid(s) comprising the tolerogenic nanoparticles described herein encode one or more of the epitopes shown in Table 20.

### Der p5 Predictions:

TABLE 21
Deposited human Der p 5 epitopes in the IEDB confirmed by T cell testing (Huang
et al. Allergy, 74: 2461-2478; Oseroff et al. (2017) Clin. Exp. Allergy, 47: 577-592;
Oseroff et al._(2012) J. Immunol. 189: 1800-1811).

| | | Starting | Ending | Antigen |
|---|---|---|---|---|
| Epitope IRI | Sequence | Position | Position | Protein |
| www.iedb.org/ | FDFLLMERIHEQIKK | 30 | 44 | Mite |
| epitope/168530 | (SEQ ID NO: 558) | | | allergen |
| | | | | Der p 5 |
| www.iedb.org/ | GELALFYLQEQINHF | 45 | 59 | Mite |
| epitope/168637 | (SEQ ID NO: 559) | | | allergen |
| | | | | Der p 5 |
| www.iedb.org/ | RKDLDIFEQYNLEMA | 95 | 109 | Mite |
| epitope/169644 | (SEQ ID NO: 560) | | | allergen |
| | | | | Der p 5 |
| www.iedb.org/ | DFLLMQRIHEQMKKG | 32 | 45 | Der p 5 |
| epitope/559105 | (SEQ ID NO: 561) | | | |
| www.iedb.org/ | FERYNVEIALKSNEI | 101 | 115 | Der p 5 |
| epitope/559407 | (SEQ ID NO: 562) | | | |
| www.iedb.org/ | LIDGVRGVLNRLMKR | 81 | 95 | Der p 5 |
| epitope/560156 | (SEQ ID NO: 563) | | | |
| www.iedb.org/ | RGVLNRLMKRTDLDI | 86 | 100 | Der p 5 |
| epitope/560783 | (SEQ ID NO: 564) | | | |
| www.iedb.org/ | VEIALKSNEILERDL | 105 | 120 | Der p 5 |
| epitope/561189 | (SEQ ID NO: 565) | | | |
| www.iedb.org/ | YQNEFDFLLMQRIHE | 26 | 40 | Der p 5 |
| epitope/561396 | (SEQ ID NO: 566) | | | |
| www.iedb.org/ | DGVRGVLDRLMQRKDLDIF | 83 | 113 | Mite |
| epitope/836909 | EQYNLEMAKKSG | | | allergen |
| | (SEQ ID NO: 567) | | | Der p 5 |
| www.iedb.org/ | DYQNEFDFLLMERIHEQIKK | 25 | 53 | Mite |
| epitope/836979 | GELALFYLQ | | | allergen |
| | (SEQ ID NO: 568) | | | Der p 5 |
| www.iedb.org/ | EQYNLEMAKKSGDILERDL | 102 | 132 | Mite |
| epitope/837164 | KKEEARVKKIEV | | | allergen |
| | (SEQ ID NO: 569) | | | Der p 5 |
| www.iedb.org/ | KKGELALFYLQEQINHFEEK | 43 | 78 | Mite |
| epitope/837734 | PTKEMKDKIVAEMDTI | | | allergen |
| | (SEQ ID NO: 570) | | | Der p 5 |

Accordingly, in certain embodiments the epitopes comprising the tolerogenic nanoparticles described herein comprises one or more of the epitopes shown in Table 21 and/or the corresponding nucleic acid sequences delivered by tolerogenic nanoparticles.

### Der p7 Predictions:

TABLE 22
Deposited human Der p 7 epitopes in the IEDB, confirmed by T cell testing
(Huang et al. (2019) Allergy, 74: 2461-2478).

| | | Starting | Ending | Parent |
|---|---|---|---|---|
| Epitope IRI | Sequence | Position | Position | Protein |
| www.iedb.org/ | DPIHYDKITEEINKAVDEAVA | 18 | 47 | Der p 7 |
| epitope/969047 | AIEKSETFD | | | |
| | (SEQ ID NO: 571) | | | |
| www.iedb.org/ | LKGELDMRNIQVRGLKQMKR | 67 | 97 | Der p 7 |
| epitope/969062 | VGDANVKSEDG | | | |
| | (SEQ ID NO: 572) | | | |
| www.iedb.org/ | TAIFQDTVRAEMTKVLAPAFK | 187 | 215 | Der p 7 |
| epitope/969079 | KELERNNQ | | | |
| | (SEQ ID NO: 573) | | | |
| www.iedb.org/ | VAAIEKSETFDPMKVPDHSDK | 37 | 67 | Der p 7 |
| epitope/969083 | FERHIGIIDL | | | |
| | (SEQ ID NO: 574) | | | |
| www.iedb.org/ | VELSLEVSEEGNMTLTSFEVR | 140 | 165 | Der p 7 |
| epitope/969084 | QFANV | | | |
| | (SEQ ID NO: 575) | | | |
| www.iedb.org/ | VHDDVVSMEYDLAYKLGDLH | 107 | 142 | Der p 7 |
| epitope/969085 | PNTHVISDIQDFVVEL | | | |
| | (SEQ ID NO: 576) | | | |
| www.iedb.org/ | VNHIGGLSILDPIFAVLSDVLT | 166 | 193 | Der p 7 |
| epitope/969086 | AIFQDT | | | |
| | (SEQ ID NO: 577) | | | |

Accordingly, in certain embodiments the epitopes comprising the tolerogenic nanoparticles described herein comprises one or more of the epitopes shown in Table 22 and/or the corresponding nucleic acid sequences delivered by these particles . . .

### Der p23 Predictions:

Der p 23, a perithrophin-like protein, is also known to induce a high IgE-levels, comparable to Der p 1 and 2.

TABLE 23
Deposited human Der p 23 epitopes in the IEDB, confirmed by T-cell testing
(Huang et al. (2019) Allergy, 74: 2461-2478; Banerjee et al. (2014) J. Immunol.
192: 4867-4875.)

| | | Starting | Ending | Antigen |
|---|---|---|---|---|
| Epitope IRI | Description | Position | Position | Name |
| www.iedb.org/ | GYFADPKDPHKFYICSNWEA | 52 | 80 | dust mite |
| epitope/226426 | VHKDCPGNT | | | allergen |
| | (SEQ ID NO: 578) | | | precursor |
| www.iedb.org/ | KFYICSNWEAVHKDCPGNTR | 62 | 90 | dust mite |
| epitope/226429 | WNEDEETCT | | | allergen |
| | (SEQ ID NO: 579) | | | precursor |
| www.iedb.org/ | GYFADPKDPHKFYICSNWEA | 52 | 90 | dust mite |
| epitope/969052 | VHKDCPGNTRWNEDEETCT | | | allergen |
| | (SEQ ID NO: 580) | | | precursor |
| www.iedb.org/ | MANDNDDDPTTTVHPTTTE | 21 | 52 | Der p23 |
| epitope/969067 | QPDDKFECPSRFG | | | allergen |
| | (SEQ ID NO: 581) | | | |
| www.iedb.org/ | PTTTEQPDDKFECPSRFGYFA | 35 | 68 | dust mite |
| epitope/969071 | DPKDPHKFYICSN | | | allergen |
| | (SEQ ID NO: 582) | | | precursor |

Accordingly, in certain embodiments the epitopes comprising the tolerogenic nanoparticles described herein comprises one or more of the epitopes shown in Table 23 and/or the corresponding nucleic acid sequences delivered by these particles . . .

In addition to epitope deposition in the IEDB, additional therapeutic candidates appear in the literature. For example, Hinz et al. (2014) Clinical & Experimental Allergy, 2015 (45) 1601-1612 analyzed patterns of immune recognition of Der p- and Der f-proteins. The authors document comprehensive T cell responses, including characterization of the Th1/Th2 balance to HDM allergens and their respective epitopes. In addition to group 1 and 2 allergens, the study also included the recently described Der p 23. This allowed the identification of a pool immunodominant epitopes that induce T cell responses ex vivo. Based on response magnitude, the immune reactivity in group 1 was the strongest, exceeding reactivity to group 2 by two-fold. Moreover, reactivity to groups 1 and 2 was >10-fold higher than the reactivity to Der p 23. This is in keeping with higher abundance of Der p 1 and Der p 2 in HDM extracts, while Der p 23 is less detectable. Tables 24-26 list the published T-cell epitope sequences, many of which are also present in our IEDB searches, listed above. We also calculated pI and GRAVY values for these peptide sequences, as described earlier. The underlined sequences represent those with the most favorable loading capacity based on solubility and charge.

TABLE 24
HDM Der p 1 human T cell epitopes.

| Epitope | Specification | Sequence | pI | GRAVY |
|---|---|---|---|---|
| Epitope 1 | 95-114 | DLRQMRTVTPIRMQGGCGSC | 9.02 | −0.385 |
| | | (SEQ ID NO: 583) | | |
| Epitope 2 | 111-130- | GCGSCWAFSGVAATESAYLA | 4.00 | 0.740 |
| | | (SEQ ID NO: 584) | | |
| Epitope 3 | 170-184 | QESYYRYVAREQSCR | 8.18 | −1.633 |
| | | (SEQ ID NO: 585) | | |
| Epitope 4 | 190-204 | RFGISNYCQIYPPNA | 8.20 | −0.393 |
| | | (SEQ ID NO: 586) | | |
| Epitope 5 | 250-264 | HAVNIVGYSNAQGVD | 5.08 | 0.040 |
| | | (SEQ ID NO: 587) | | |
| Epitope 6 | 160-174 | IEYIQHNGVVQESYY | 4.51 | −0.560 |
| | | (SEQ ID NO: 588) | | |
| Epitope 7 | 36-50 | VKYVQSNGGAINHLS | 8.57 | −0.187 |
| | | (SEQ ID NO: 589) | | |
| Epitope 8 | 205-219 | NKIREALAQTHSAIA | 8.75 | −0.240 |
| | | (SEQ ID NO: 590) | | |
| Epitope 9 | 76-94 | DLNAETNACSINGNAPAEI | 3.57 | −0.289 |
| | | (SEQ ID NO: 591) | | |
| Epitope 10 | 225-244 | KDLDAFRHYDGRTIIQRDNG | 6.75 | −1.350 |
| | | (SEQ ID NO: 592) | | |
| Epitope 11 | 141-160 | QELVDCASQHGCHGDTIPRG | 5.21 | −0.670 |
| | | (SEQ ID NO: 593) | | |
| Epitope 12 | 275-289 | NWGDNGYGYFAANID | 3.56 | −0.753 |
| | | (SEQ ID NO: 594) | | |
| Epitope 13 | 220-234 | VIIGIKDLDAFRHYD | 5.30 | 0.153 |
| | | (SEQ ID NO: 595) | | |
| Epitope 14 | 46-60 | INHLSDLSLDEFKNR | 5.38 | −0.800 |
| | | (SEQ ID NO: 596) | | |
| Epitope 15 | 285-299 | AANIDLMMIEEYPYV | 3.57 | 0.413 |
| | | (SEQ ID NO: 597) | | |

Accordingly, in certain embodiments the epitopes comprising the tolerogenic nanoparticles described herein comprises one or more of the epitopes shown in Table 24 and/or the corresponding nucleic acids for particle delivery. C

TABLE 25
HDM DER P 2 human t cell epitopes.

| Epitope | Specifications | Sequence | pI | GRAVY |
|---|---|---|---|---|
| Epitope 1 | 21-40 | CHGSEPCIIHRGKPFQLEAV | 6.91 | −0.175 |
| | | (SEQ ID NO: 598) | | |
| Epitope 2 | 86-105 | YDIKYTWNVPKIAPKSENV | 8.38 | −0.525 |
| | | V | | |
| | | (SEQ ID NO: 599) | | |
| Epitope 3 | 46-60 | NTKTAKIEIKASIDG | 8.50 | −0.513 |
| | | (SEQ ID NO: 600) | | |
| Epitope 4 | 115-129 | GVLACAIATHAKIRD | 8.23 | 0.700 |
| | | (SEQ ID NO: 601) | | |
| Epitope 5 | 1-15 | DQVDVKDCANHEIKK | 5.43 | −1.247 |
| | | (SEQ ID NO: 602) | | |
| Epitope 6 | 36-50 | QLEAVFEANQNTKTA | 4.53 | −0.673 |
| | | (SEQ ID NO: 603) | | |
| Epitope 7 | 81-95 | VKGQQYDIKYTWNVP | 8.40 | −1.007 |
| | | (SEQ ID NO: 604) | | |
| Epitope 8 | 71-85 | NACHYMKCPLVKGQQ | 8.86 | −0.540 |
| | | (SEQ ID NO: 605) | | |

Accordingly, in certain embodiments the epitopes comprising the tolerogenic nanoparticles described herein comprises one or more of the epitopes shown in Table 25 and/or the corresponding nucleic acid sequences for particle delivery.

TABLE 26
HDM DER P23 human T cell epitopes.

| Epitope | Specifications | Sequence | pI | GRAVY |
|---|---|---|---|---|
| Epitope 1 | 36-55 | PKDPHKFYICSNWEAVHKDC | 7.23 | −1.000 |
| | | (SEQ ID NO: 606) | | |
| Epitope 2 | 26-40 | CPSRFGYFADPKDPH | 6.74 | −1.067 |
| | | (SEQ ID NO: 607) | | |

Accordingly, in certain embodiments the epitopes comprising the tolerogenic nanoparticles described herein comprises one or more of the epitopes shown in Table 26 and/or the corresponding nucleic acid sequences for particle delivery.

Huang et al. (2019) Allergy, 74:2461-2478 assembled a panel of 33 peptides ranging from 27 to 43 amino acids, which cover the dominant T-cell recognition sites in Der p 1, 2, 5, 7, 21, and 23. These peptides and the complete allergen molecules were used to study allergen/peptide-specific IgE and IgG responses, as well as T cell and cytokine responses to the allergens and peptides in HDM-allergic patients vs. subjects without HDM sensitization. The study identified a cocktail of 31 synthetic peptides containing T cell epitopes representative of the six important HDM allergens (Table 10).

TABLE 27
HDM DER P 1, P 2, P 5, P 7, P 21, P 23 human T-cell epitopes.

| | | No. of |
|---|---|---|
| | Sequence (N′-C′) | aa |
| Der p 1 | TNACSINGNAPAEIDLRQMRTVTPIRMQGGCGSCWAFSGVA | 41 |
| | (SEQ ID NO: 608) | |
| | ATESAYLAYRNQSLDLAEQELVDCASQHGCHGDTIPRGIEYIQ | 43 |
| | (SEQ ID NO: 609) | |
| | HNGVVQESYYRYVAREQSCRRPNAQRFGISN | 31 |
| | (SEQ ID NO: 610) | |
| | REQSCRRPNAQRFGISNYCQIYPPNVNKIREALAQTH | 37 |
| | (SEQ ID NO: 611) | |
| | KDLDAFRHYDGRTIIQRDNGYQPNYHAVNIV | 31 |
| | (SEQ ID NO: 612) | |
| | GRTIIQRDNGYQPNYHAVNIVGYSNAQGVDYWI | 33 |
| | (SEQ ID NO: 613) | |
| | VGYSNAQGVDYWIVRNSWDTNWGDNGYGYFAANI | 34 |
| | (SEQ ID NO: 614) | |
| | VRNSWDTNWGDNGYGYFAANIDIMMIEEYPYVVIL | 35 |
| | (SEQ ID NO: 615) | |
| Der p 2 | DQVDVKDCANHEIKKVLVPGCHGSEPCIIHRGK | 33 |
| | (SEQ ID NO: 616) | |
| | CHGSEPCIIHRGKPFQLEAVFEANQNSKTAK | 31 |
| | (SEQ ID NO: 617) | |
| | EANQNSKTAKIEIKASIEGLEVDVPGIDPNAC | 32 |
| | (SEQ ID NO: 618) | |
| | EVDVPGIDPNACHYMKCPLVKGQQYDIKYTWIVPKIAPKSEN | 42 |
| | (SEQ ID NO: 619) | |
| | APKSENVVVTVKVMGDNGVLACAIATHAKIRD | 32 |
| | (SEQ ID NO: 620) | |
| Der p 5 | CDYQNEFDFLLMERIHEQIKKGELALFYLQ | 30 |
| | (SEQ ID NO: 621) | |
| | CKKGELALFYLQEQINHFEEKPTKEMKDKIVAEMDTI | 37 |
| | (SEQ ID NO: 622) | |
| | CDGVRGVLDRLMQRKDLDIFEQYNLEMAKKSG | 32 |
| | (SEQ ID NO: 623) | |
| | CEQYNLEMAKKSGDILERDLKKEEARVKKIEV | 32 |
| | (SEQ ID NO: 624) | |
| Der p 7 | CDPIHYDKITEEINKAVDEAVAAIEKSETFD | 31 |
| | (SEQ ID NO: 625) | |
| | CVAAIEKSETFDPMKVPDHSDKFERHIGIIDL | 32 |
| | (SEQ ID NO: 626) | |
| | CLKGELDMRNIQVRGLKQMKRVGDANVKSEDG | 32 |
| | (SEQ ID NO: 627) | |
| | CVHDDVVSMEYDLAYKLGDLHPNTHVISDIQDFVVEL | 37 |
| | (SEQ ID NO: 628) | |
| | CVELSLEVSEEGNMTLTSFEVRQFANV | 27 |
| | (SEQ ID NO: 629) | |
| | CVNHIGGLSILDPIFAVLSDVLTAIFQDT | 29 |
| | (SEQ ID NO: 630) | |
| | CTAIFQDTVRAEMTKVLAPAFKKELERNNQ | 30 |
| | (SEQ ID NO: 631) | |
| Der p 21 | CFIVGDKKEDEWRMAFDRLMMEELETKIDQVEKGL | 35 |
| | (SEQ ID NO: 632) | |
| | CLHLSEQYKELEKTKSKELKEQILRELTIGENFMKGAL | 38 |
| | (SEQ ID NO: 633) | |
| | CMKGALKFFEMEAKRTDLNMFERYNYEFAL | 30 |
| | (SEQ ID NO: 634) | |
| | CYNYEFALESIKLLIKKLDELAKKVKAVNPDEYY | 34 |
| | (SEQ ID NO: 635) | |
| Der p 23 | MANDNDDDPTTTVHPTTTEQPDDKFECPSRFG | 32 |
| | (SEQ ID NO: 636) | |
| | PTTTEQPDDKFECPSRFGYFADPKDPHKFYICSN | 34 |
| | (SEQ ID NO: 637) | |
| | GYFADPKDPHKFYICSNWEAVHKDCPGNTRWNEDEETCT | 39 |
| | (SEQ ID NO: 638) | |
| | GYFADPKDPHKFYICSNWEAVHKDCPGNT | 29 |
| | (SEQ ID NO: 639) | |
| | KFYICSNWEAVHKDCPGNTRWNEDEETCT | 29 |
| | (SEQ ID NO: 640) | |

Accordingly, in certain embodiments the epitopes comprising the tolerogenic nanoparticles described herein comprises one or more of the epitopes shown in Table 27 and/or the nucleic acid(s) comprising the tolerogenic nanoparticles described herein encode one or more of the epitopes shown in Table 27.

Hoyne et al. (1993) J. Exp. Med. 178 (5): 1783-1788 used a murine model of T-cell recognition for assess Der p1 epitopes that are capable of infecting T-cell function in vivo. The results demonstrated that inhalation of low peptide concentrations of the major Der p1 T-cell epitope (residues 111-139) was capable of inducing tolerance in naïve C57BL/6J mice. These animals become profoundly unresponsive to an immunogenic challenge with the intact allergen. When restimulated in vitro with antigen, lymphnode T-cells isolated from tolerant mice secreted very low levels of IL-2, proliferated poorly, and were unable to support antibody production. Furthermore, intranasal peptide therapy was able to inhibit an ongoing immune response to the allergen in mice, indicating the possibility for developing allergen-based immunotherapy.

### Selection of Human Der p Epitopes for Delivery by Tolerogenic Nanoparticles

Based on the data compilation for Der p allergens in Tables 24-26, we selected a short list of Der p 1, 2, and 23 epitopes (underlined items) with strong T-cell reactivity and favorable loading characteristics as lead candidates for incorporation into PLGA nanoparticles (Table 12). We propose that these epitopes can also be used for formulating multi-epitope peptide and mRNA tolerogenic nanoparticles.

TABLE 28
Human Der P1 T-cell epitope predictions for synthesis of PLGA nanoparticles.

| No. | Allergen | Specifications | Sequence | pI | GRAVY |
|---|---|---|---|---|---|
| 1 | Der p 1 | 170-184 | QESYYRYVAREQSCR | 8.18 | −1.633 |
| | | | (SEQ ID NO: 641) | | |
| 2 | Der p 1 | 225-244 | KDLDAFRHYDGRTIIQRD | 6.75 | −1.350 |
| | | | NG | | |
| | | | (SEQ ID NO: 642) | | |
| 3 | Der p 2 | 1-15 | DQVDVKDCANHEIKK | 5.43 | −1.247 |
| | | | (SEQ ID NO: 643) | | |
| 4 | Der p 2 | 81-95 | VKGQQYDIKYTWNVP | 8.40 | −1.007 |
| | | | (SEQ ID NO: 644) | | |
| 5 | Der p 23 | 36-55 | PKDPHKFYICSNWEAVH | 7.23 | −1.000 |
| | | | KDC | | |
| | | | (SEQ ID NO: 645) | | |
| 6 | Der p 23 | 26-40 | CPSRFGYFADPKDPH | 6.74 | −1.067 |
| | | | (SEQ ID NO: 646) | | |

Accordingly, in certain embodiments the epitopes comprising the tolerogenic nanoparticles described herein comprises one or more of the epitopes shown in Table 28 and/or the corresponding nucleic acid sequences for particle delivery . . .

To develop the mRNA vaccine, above peptide sequences were translated into cDNA nucleotide sequences that can be combined for developing a multi-epitope vaccine as described herein.

TABLE 29
Reverse translation of Der p 1 peptide into internucleotide sequences for
developing multi-epitope mRNA delivering nanoparticles.

| No. | Allergen | Location | Sequence | cDNA sequence |
|---|---|---|---|---|
| 1 | Der p 1 | 170-184 | QESYYRYVAREQSCR | caggaaagctattatcgctatgtggcgcg |
| | | | (SEQ ID NO: 647) | cgaacagagctgccgc |
| | | | | (SEQ ID NO: 648) |
| 2 | Der p 1 | 225-244 | KDLDAFRHYDGRTIIQR | aaagatctggatgcgtttcgccattatgat |
| | | | DNG | ggccgcaccattattcagcgcgataacg |
| | | | (SEQ ID NO: 649) | gc |
| | | | | (SEQ ID NO: 650) |
| 3 | Der p 2 | 1-15 | DQVDVKDCANHEIKK | Gatcaggtggatgtgaaagattgcgcg |
| | | | (SEQ ID NO: 651) | aaccatgaaattaaaaaa |
| | | | | (SEQ ID NO: 652) |
| 4 | Der p 2 | 81-95 | VKGQQYDIKYTWNVP | gtgaaaggccagcagtatgatattaaata |
| | | | (SEQ ID NO: 653) | tacctggaacgtgccg |
| | | | | (SEQ ID NO: 654) |
| 5 | Der p 23 | 36-55 | PKDPHKFYICSNWEAVH | ccgaaagatccgcataaattttatatttgc |
| | | | KDC | agcaactgggaagcggtgcataaagatt |
| | | | (SEQ ID NO: 655) | gc |
| | | | | (SEQ ID NO: 656) |
| 6 | Der p 23 | 26-40 | CPSRFGYFADPKDPH | tgcccgagccgctttggctattttgcggat |
| | | | (SEQ ID NO: 657) | ccgaaagatccgcat |
| | | | | (SEQ ID NO: 658) |

Using the approach described above for peanut antigen nucleic acids, an integrated coding sequence was developed by adding cleavable linker sequences to string together the proposed tolerogenic epitopes downstream of the human transferrin receptor 1 (1-118) targeting domain. After the addition of start and stop codons, codon optimization was performed to allow human expression (Table 30). The coding sequence will be inserted into a pTNT plasmid for mRNA synthesis.

TABLE 30
Codon-optimized coding sequence for human Der p 1,
p 2, and p 23 T cell epitopes (based on Table 29).
Complete coding sequence with human TfR1-118
(cyan), spliced in upstream of human Der p 1, p 2,
p 23 T-cell epitopes, which are separated by
cleavable linkers (yellow and green). After the
addition of start (purple) and stop codons (red),
codon optimization was performed. This yielded
an average GC content of 61.45%.

ATGATGGACCAGGCCAGAAGCGCCTTCTCCAACCTCTTCGGAGGCGAGCC
TCTGAGCTACACCAGATTCAGCCTGGCTAGACAGGTGGATGGCGACAACA
GCCACGTGGAAATGAAGCTGGCTGTGGACGAGGAAGAGAACGCCGATAA
CAACACCAAGGCCAATGTGACAAAGCCTAAAAGATGCAGCGGCTCCATCT
GCTACGGCACAATCGCCGTGATCGTGTTCTTCCTGATCGGATTTATGATCG
GCTACCTGGGCTACTGTAAAGGCGTGGAACCTAAGACCGAGTGCGAGAGA
CTGGCTGGCACAGAGAGCCCAGTGCGGGAAGAACCAGGAGAAGATTTCC
CCGCCGGCGGCGGCGGATCTAGAGTGCGGAGAGGAGGAGGCGGCAGCCA
GGAGTCTTATTACAGATACGTGGCCAGAGAGCAGAGCTGCAGAGGCGGC
GGAGGCTCTAGAGTGCGGCGGGGCGGCGGGGGCAGCAAGGATCTGGACG
CCTTTAGACACTACGACGGCAGAACCATTATCCAGCGGGACAATGGCGGC
GGCGGTGGCAGCAGAGTGCGGCGGGGAGGCGGCGGCTCTGATCAGGTTG
ACGTGAAGGACTGTGCCAACCACGAGATCAAAAAGGGCGGCGGTGGCAG
CAGAGTGAGAAGAGGCGGCGGGGGCAGCGTGAAGGGCCAGCAATACGAC
ATCAAGTACACCTGGAACGTGCCCGGAGGCGGAGGCAGCCGCGTCAGAA
GGGGCGGTGGAGGCTCTCCTAAAGACCCCCACAAGTTCTACATCTGCAGC
AACTGGGAGGCCGTGCACAAGGACTGCGGCGGCGGCGGCAGCAGAGTCC
GACGTGGCGGAGGCGGCTCCTGTCCTAGCCGGTTCGGCTATTTCGCCGAC
CCTAAGGATCCTCATCGGGTGCGGAGATGA (SEQ ID NO: 659)

The NIH-supported IEDB (IEDB.org) resource was used to predict mouse T-cell epitopes after entry of Dep p1, p2 and p23 protein sequences into the database (Table 13). The epitope predictions included looking at binding affinity to the murine H2-IAb allele (MHC-II), which is expressed in C57/B16 mice. This allowed the identification of 15-mer non-IgE binding T-cell epitopes, among which the sequences with the highest (top 20%) MHC II binding affinity were selected (Tables 31-33). The selection was complemented by also assessing the pI and GRAVY values for prediction of peptides with the best chance for PLGA loading capacity.

### 1 Der p1 Predictions:

The intact Der p1 protein sequence is:

(SEQ ID NO: 660)
ARPSSIKTFEEYKKAFNKSYATFEDEEAARKNFLESVKYVQSNGGAINH
LSDLSLDEFKNRFLMSAEAFEHLKTQFDLNAETNACSINGNAPAEIDLR
QMRTVTPIRMQGGCGSCWAFSGVAATESAYLAYRNQSLDLAEQELVDCA
SQHGCHGDTIPRGIEYIQHNGVVQESYYRYVAREQSCRRPNAQRFGISN
YCQIYPPNVNKIREALAQTHSAIAVIIGIKDLDAFRHYDGRTIIQRDNG
YQPNYHAVNIVGYSNAQGVDYWIVRNSWDTNWGDNGYGYFAANIDLMMI
EEYPYVVIL.

TABLE 31
Der p 1 epitope predictions for C57/BL6 mouse (H2 IAb).

| No. | Epitope | Location | Sequence | pI | GRAVY |
|---|---|---|---|---|---|
| 1 | Epitope 1 | 111-125 | GCGSCWAFSGVAATE | 4.0 | 0.633 |
| | | | (SEQ ID NO: 661) | | |
| 2 | Epitope 2 | 279-293 | DNGYGYFAANIDLMM | 3.56 | 0.073 |
| | | | (SEQ ID NO: 662) | | |
| 3 | Epitope 3 | 208-222 | IREALAQTHSAIAVI | 6.75 | 0.833 |
| | | | (SEQ ID NO: 663) | | |
| 4 | Epitope 4 | 195-209 | SNYCQIYPPNVNKIR | 9.19 | −0.887 |
| | | | (SEQ ID NO: 664) | | |
| 5 | Epitope 5 | 36-50 | SVKYVQSNGGAINHL | 8.33 | −0.187 |
| | | | (SEQ ID NO: 665) | | |
| 6 | Epitope 6 | 10-24 | EEYKKAFNKSYATFE | 6.32 | −1.373 |
| | | | (SEQ ID NO: 666) | | |

Accordingly, in certain embodiments the epitopes comprising the tolerogenic nanoparticles described herein comprises one or more of the epitopes shown in Table 31 and/or the corresponding nucleic acid sequences for particle delivery.

### Der p2 Predictions:

The intact Der p 2 protein sequence is:

(SEQ ID NO: 667)
DQVDVKDCANHEIKKVLVPGCHGSEPCIIHRGKPFQLEAVFEANQNTKT
AKIEIKASIDGLEVDVPGIDPNACHYMKCPLVKGQQYDIKYTWNVPKIA
PKSENVVVTVKVMGDDGVLACAIATHAKIRD.

TABLE 32
Der p 2 mouse T cell epitope predictions for C57/BL6 mouse (H2 IAb)

| No. | Epitope | Location | Sequence | pI | GRAVY |
|---|---|---|---|---|---|
| 1 | Epitope 2 | 87-101 | DIKYTWNVPKIAPKS | 9.53 | −0.707 |
| | | | (SEQ ID NO: 668) | | |
| 2 | Epitope 4 | 113-127 | DDGVLACAIATHAKI | 5.21 | 0.767 |
| | | | (SEQ ID NO: 669) | | |
| 3 | Epitope 6 | 37-51 | LEAVFEANQNTKTAK | 6.14 | −0.700 |
| | | | (SEQ ID NO: 670) | | |

Accordingly, in certain embodiments the epitopes comprising the tolerogenic nanoparticles described herein comprises one or more of the epitopes shown in Table 32 and/or the corresponding nucleic acid sequences for particle delivery.

### Der p23 Predictions:

The intact Der p 23 protein sequence is:

(SEQ ID NO: 671)
ANDNDDDPTTTVHPTTTEQPDDKFECPSRFGYFADPKDPHKFYICSNWE
AVHKDCPGNTRWNEDEETCT.

TABLE 33
Der P 23 mouse T cell epitope predictions for C57/BL6 mouse (H2 IAb)

| No. | Epitope | Location | Sequence | pI | GRAVY |
|---|---|---|---|---|---|
| 1 | Epitope 1 | 6-20 | DDPTTTVHPTTTEQP | 4.02 | −1.467 |
| | | | (SEQ ID NO: 672) | | |
| 2 | Epitope 2 | 28-42 | SRFGYFADPKDPHKF | 8.24 | −1.200 |
| | | | (SEQ ID NO: 673) | | |

Accordingly, in certain embodiments the epitopes comprising the tolerogenic nanoparticles described herein comprises one or more of the epitopes shown in Table 33 and/or the corresponding nucleic acid sequences for particle delivery . . .

Based on the epitope selections, a cDNA sequence was designed for murine use, using the sequential process of reverse translation, linker addition, insertion of a mouse transferrin receptor 1 (1-118) targeting sequence, addition of start and stop codons, and codon optimization. The nucleotide sequence is inserted into a pTNT plasmid that will be used for the synthesis of a multi-epitope mRNA, which can be encapsulated in cationic lipid or PLGA nanoparticles, as described herein.

TABLE 34
Reverse translation of HDM T cell epitopes for mRNA preparation.

| No | Epitope | Location | Reverse translated cDNA sequence |
|---|---|---|---|
| 1 | Der p 1 | 195-209 | agcaactattgccagatttatccgccgaacgtgaacaaaattcgc |
| | | | (SEQ ID NO: 674) |
| 2 | Der p 1 | 10-24 | gaagaatataaaaaagcgtttaacaaaagctatgcgacctttgaa |
| | | | (SEQ ID NO: 675) |
| 3 | Der p 2 | 87-101 | gatattaaatatacctggaacgtgccgaaaattgcgccgaaaagc |
| | | | (SEQ ID NO: 676) |
| 4 | Der p 2 | 37-51 | ctggaagcggtgtttgaagcgaaccagaacaccaaaaccgcgaaa |
| | | | (SEQ ID NO: 677) |
| 5 | Der p 23 | 6-20 | gatgatccgaccaccaccgtgcatccgaccaccaccgaacagccg |
| | | | (SEQ ID NO: 678) |
| 6 | Der p 23 | 28-42 | agccgctttggctattttgcggatccgaaagatccgcataaattt |
| | | | (SEQ ID NO: 679) |

Table 35 shows a codon-optimized multi-epitope nucleotide sequence for murine use.

TABLE 35
2. Codon optimized cDNA sequence encoding for the
mouse TFR 1-118 and DER P 1, 2, 23 T cell
epitopes. Complete coding sequence with human
TfR1-118 domain (cyan), spliced in upstream of
murine Der p 1, p 2, p 23 T-cell epitopes. The
epitopes was linked by including cleavable
linkers (yellow and green). After the addition of
start (purple) and stop codons (red), codon
optimization was performed. This yielded a mRNA
strand with an average GC content of 55.59%.

ATGATGATGGACCAGGCTCGCAGTGCTTTCAGTAACCTGTTTGGAGGGG
AGCCCCTATCCTACACCAGGTTCAGCTTGGCCAGGCAAGTGGATGGAGA
CAATTCACATGTGGAGATGAAACTTGCCGCGGATGAGGAGGAAAATGCC
GACAACAATATGAAGGCATCAGTGCGGAAACCAAAGCGCTTCAATGGCA
GGCTGTGCTTTGCTGCTATAGCCCTGGTTATTTTCTTCCTCATTGGCTT
CATGTCCGGCTATTTAGGGTATTGTAAGAGAGTAGAACAAAAAGAAGAG
TGTGTGAAGCTGGCAGAGACTGAGGAGACAGATAAAAGCGAGACCATGG
AGACAGAAGATGTGGGCGGAGGCGGGTCCCGGGTGAGACGTGGGGGAGG
TGGAAGTTCAAACTACTGCCAGATCTACCCTCCGAATGTGAACAAGATC
CGCGGGGGTGGCGGGAGCCGGGTCCGACGCGGGGGAGGTGGGTCTGAAG
AATACAAGAAGGCCTTCAACAAATCTTATGCGACCTTTGAGGGCGGTGG
CGGGAGCAGAGTCAGACGGGGAGGGGGCGGAAGCGACATCAAGTACACC
TGGAATGTTCCTAAAATTGCCCCTAAGTCTGGCGGGGGCGGATCGAGGG
TCAGAAGAGGCGGAGGCGGGTCTCTCGAAGCAGTATTTGAAGCCAACCA
GAACACCAAGACAGCTAAAGGTGGGGGAGGTTCCAGGGTCAGGCGCGGA
GGTGGTGGCTCTGATGATCCTACAACTACTGTTCACCCAACTACGACGG
AGCAGCCCGGAGGCGGTGGCTCCCGTGTGAGACGAGGTGGAGGCGGGTC
AAGTCGATTTGGCTATTTTGCAGACCCAAAAGACCCCCACAAGTTCCGA
GTCCGGCGGTGA (SEQ ID NO: 680)

### Tolerance to Overcome Anti-Drug Antibodies to Adalimumab Through Delivery of T-Cell Epitopes.

Since the first monoclonal antibody (mAb) was approved by the United States Food and Drug Administration (US FDA) in 1986, therapeutic antibodies have become a major strategy in oncology and clinical immunology due to paradigm shift approaches to antibody engineering. Current antibody therapeutics are designed to progressively reduce or eliminate adverse effects through safe design procedures. As a result, therapeutic antibodies have become one of the preeminent classes of drug development, allowing antibodies to become the best-selling drug category in the pharmaceutical market. Eight of the top ten bestselling drugs in 2018 were biologics, with a global market evaluation of US $115.2 billion, and expecting revenues or $300 billion by 2025. Accordingly, the market for therapeutic antibodies has experienced explosive growth, including for the treatment of cancer, autoimmune, metabolic and infectious diseases. As of December 2019, 79 therapeutic mAbs have been approved by the US FDA, with the potential for additional expansion (Lu et al. (2020) J. Biomed. Sci. 27:1). Key examples are tumor necrosis factor (TNF) inhibitors, including Etanercept (Enbrel), Infliximab (Remicade), Adalimumab (Humira), Certolizumab, and Golimumab (Simponi). Among these, Humira and Remicade have become blockbuster drugs, with with global sales of US $17.6 and $5.9 billion, respectively. Moreover, these drugs are closely followed by Enbrel, with global sales of USS 5.8 Billion (//www.researchandmarkets.com/research/6k8r46/tumor_necrosis?w=5). The development of these antibodies reflect the key role of TNF in many autoimmune diseases, including rheumatoid arthritis (RA), juvenile arthritis, psoriatic arthritis, plaque psoriasis, ankylosing spondylitis, ulcerative colitis (UC), Crohn's disease, and cancer.

In spite of this therapeutic success story, the efficacy of some of these antibodies have been hampered by the development of neutralizing antidrug antibodies (ADAs) that reduce the therapeutic impact. This includes the ability of the ADAs to exert neutral effects that interfere in the pharmacokinetics and biodistribution of the therapeutic antibodies, as well as the ability to generate hypersensitivity reactions, including life-threatening anaphylaxis (Kishimoto (2020) Front. Immunol. 11:969). Although almost all therapeutic antibodies currently developed are humanized, the extent of the humanization is varied. For example, while chimeric antibodies are humanized with respect to constant (C) domains, the variable (V) regions often include sequences from murine origin (see, e.g., FIG. 6). In addition, humanized antibodies are frequently modified in the complementarity-determining regions (CDR) regions of the V and H domains, with the potential to be immunogenic in spite of being from humanized origin or obtained from immunoglobulin sequences expressed in humanized mice. Thus, none of these approaches fully guarantee the absence of immunogenicity. Not only do chimeric antibodies such as rituximab (Rtx) and infliximab (Ifx) induce specific ADAs that lead to a reduction in clinical efficacy, but similar outcomes out also been seen with humanized antibodies such as alemtuzumab and vedolizumab. While humanization of the C-domain in therapeutic antibodies clearly reduce the occurrence of ADA, the benefit of humanizing the V-domains remain problematic due to the lack of knowledge about the molecular determinants that can contribute to immunogenicity (see, e.g., Hamze et al. (2017) Front. Immunol. 8:500; Bendtzen et al. (2015) Front. Immunol. 6:109). One possibility is that the CDRs in the antibody V domain acquire unique amino acid sequences to become immunogenic at antibody sites known as idiotopes. These regions can induce the development of anti-idiotope antibodies, with the assistance of helper/follicular dendritic T-cells; the T-cells play a role in promoting immunoglobulin gene rearrangement, development of junctional diversity and somatic hypermutation in maturing B-cells. The immunogenic Ig sequences in the CDR serve as T-cell epitopes, which play a role in the development of ADA's (FIG. 6),

The identification of T-cell epitopes in immunoglobulin CDR regions that promote the development of ADAs presents a potential breakthrough in the use of these peptide sequences to develop Treg-generating tolerogenic nanoparticles (Hamze et al. (2017) Front. Immunol. 8:500; Meunier et al. (2020) Cellular & Molecular Immunol. 2020, 17:656-658). The use of liver-targeting tolerogenic nanoparticles that deliver ADA inducing T-cell epitopes to LSECs, could serve to suppress the ADA responses.

Described below are various T cell-epitopes, which have been identified in promoting the ADA response to the therapeutic antibody, Adalimumab (Adm, aka. Humira). Humira is widely used for the treatment of autoimmune disease processes. The discovery began by collecting data on adalimumab T-cell epitopes, appearing in the literature, plus further refinement based on predictions for MHC-II binding in the IEDB. Moreover, we also used our software to calculate peptide p1 and GRAVY characterist values to predict PLGA encapsulation. In addition to collecting peptide data, we also performed reverse translation to design a multi-epitope mRNA vaccine to treat adalimumab ADA. This vaccine serves as a prototype of similar vaccines that can be designed to treat ADA responses to other therapeutic antibodies.

Meunier et al. (2017) Cell Mol Immunol. 2020; 17:656-658 identified adalimumab CD4 T-cell epitopes, using human T-cell lines that interact with autologous DCs, prior loaded with the immunogenic immunoglobulin peptide regions (Id.). This allowed them to generate 28 T-cell lines responsive to antibody peptides presented by DC from 14 different donors. The study also included coverage of diverse HLA-DR allotypes. The main antigenic determinants contributing to the generation of T-cell responses were CDR peptide sequences, as discussed above. This allowed the identification of 11 peptide sequences clustered in the HCDR2, HCDR3, and LCDR2 regions, among which two peptides from HCDR3 (AH91-110 and AH96-110) were demonstrated to be recognized by the majority of T-cell lines isolated from 10 different donors (see, e.g., Table 36).

TABLE 36
Sequences recognized by the majority of T cell
lines identified by Meunier et al.

| Epitope | Sequence | SEQ ID NO: |
|---|---|---|
| AH91-110 | TAVYYCAKVSYLSTASSLDY | 681 |
| AH 96-110 | CAKVSYLSTASSLDY | 682 |

Accordingly, in certain embodiments the epitopes comprising the tolerogenic nanoparticles described herein comprises one or more of the epitopes shown in Table 36 and/or the corresponding nucleic acid sequences for particle loading.

Additional prediction making was performed in the IEDB resource to identify adalimumab T-cell epitopes with high binding affinity for human HLA-DR, HLA-DP, or HLA-DQ. The following tables list human T-cell epitope predictions for adalimumab heavy chain and light chains, which can be considered for tolerogenic therapy. We demonstrate use of these sequences to identify lead epitopes that can be encapsulated in tolerogenic nanoparticles.

The Adalimumab heavy chain intact protein sequence is EVOLVESGGGLVQPGSLRLSCAASGFTFDDYAMHWVRQAPGKGLEWVSAITWNSG HIDYADSVEGRFTISRDNAKNSLYLQMNSLRAEDTAVYYCAKVSYLSTASSLDYWG QGTLVTVSSASTKGPSVF (SEQ ID NO:683). This sequence was used for predicting interactions with the human MHC-II reference set in the IEDB. We list the top 5% of predictions based on the strength of the affinity binding interactions (see Table 37).

TABLE 37
Human T cell epitope predictions for Adm heavy chain, using the IEDB database.

| | | | | Percentile |
|---|---|---|---|---|
| Allele | Start | End | Sequence | Rank |
| HLA-DRB1*04:01 | 74 | 88 | AKNSLYLQMNSLRAE | 0.03 |
| | | | (SEQ ID NO: 684) | |
| HLA-DRB1*04:01 | 75 | 89 | KNSLYLQMNSLRAED | 0.03 |
| | | | (SEQ ID NO: 685) | |
| HLA-DRB1*04:01 | 73 | 87 | NAKNSLYLQMNSLRA | 0.03 |
| | | | (SEQ ID NO: 686) | |
| HLA-DRB1*04:01 | 76 | 90 | NSLYLQMNSLRAEDT | 0.03 |
| | | | (SEQ ID NO: 687) | |
| HLA-DRB1*04:01 | 77 | 91 | SLYLQMNSLRAEDTA | 0.03 |
| | | | (SEQ ID NO: 688) | |
| HLA-DRB1*01:01 | 74 | 88 | AKNSLYLQMNSLRAE | 0.10 |
| | | | (SEQ ID NO: 689) | |
| HLA-DRB1*01:01 | 75 | 89 | KNSLYLQMNSLRAED | 0.10 |
| | | | (SEQ ID NO: 690) | |
| HLA-DRB1*01:01 | 73 | 87 | NAKNSLYLQMNSLRA | 0.10 |
| | | | (SEQ ID NO: 691) | |
| HLA-DRB1*01:01 | 76 | 90 | NSLYLQMNSLRAEDT | 0.10 |
| | | | (SEQ ID NO: 692) | |
| HLA-DRB1*01:01 | 77 | 91 | SLYLQMNSLRAEDTA | 0.10 |
| | | | (SEQ ID NO: 693) | |
| HLA-DRB1*08:02 | 111 | 125 | GQGTLVTVSSASTKG | 0.26 |
| | | | (SEQ ID NO: 694) | |
| HLA-DRB1*08:02 | 112 | 126 | QGTLVTVSSASTKGP | 0.26 |
| | | | (SEQ ID NO: 695) | |
| HLA-DRB1*08:02 | 113 | 127 | GTLVTVSSASTKGPS | 0.27 |
| | | | (SEQ ID NO: 696) | |
| HLA-DRB1*04:01 | 78 | 92 | LYLQMNSLRAEDTAV | 0.55 |
| | | | (SEQ ID NO: 697) | |
| HLA-DRB1*04:01 | 79 | 93 | YLQMNSLRAEDTAVY | 0.63 |
| | | | (SEQ ID NO: 698) | |
| HLA-DRB1*04:05 | 95 | 109 | CAKVSYLSTASSLDY | 0.64 |
| | | | (SEQ ID NO: 699) | |
| HLA-DRB1*04:05 | 94 | 108 | YCAKVSYLSTASSLD | 0.68 |
| | | | (SEQ ID NO: 700) | |
| HLA-DRB1*04:05 | 96 | 110 | AKVSYLSTASSLDYW | 0.74 |
| | | | (SEQ ID NO: 701) | |
| HLA-DRB1*08:02 | 110 | 124 | WGQGTLVTVSSASTK | 0.74 |
| | | | (SEQ ID NO: 702) | |
| HLA-DRB1*04:05 | 97 | 111 | KVSYLSTASSLDYWG | 0.77 |
| | | | (SEQ ID NO: 703) | |
| HLA-DRB1*04:05 | 98 | 112 | VSYLSTASSLDYWGQ | 0.81 |
| | | | (SEQ ID NO: 704) | |
| HLA-DRB1*01:01 | 78 | 92 | LYLQMNSLRAEDTAV | 0.91 |
| | | | (SEQ ID NO: 705) | |
| HLA-DRB1*01:01 | 79 | 93 | YLQMNSLRAEDTAVY | 0.91 |
| | | | (SEQ ID NO: 706) | |
| HLA-DRB1*04:01 | 96 | 110 | AKVSYLSTASSLDYW | 1.10 |
| | | | (SEQ ID NO: 707) | |
| HLA-DRB5*01:01 | 74 | 88 | AKNSLYLQMNSLRAE | 1.60 |
| | | | (SEQ ID NO: 708) | |
| HLA-DRB1*04:01 | 95 | 109 | CAKVSYLSTASSLDY | 1.60 |
| | | | (SEQ ID NO: 709) | |
| HLA-DRB5*01:01 | 75 | 89 | KNSLYLQMNSLRAED | 1.60 |
| | | | (SEQ ID NO: 710) | |
| HLA-DRB5*01:01 | 76 | 90 | NSLYLQMNSLRAEDT | 1.60 |
| | | | (SEQ ID NO: 711) | |
| HLA- | 21 | 35 | CAASGFTFDDYAMHW | 1.80 |
| DQA1*01:01/DQB1*05:01 | | | (SEQ ID NO: 712) | |
| HLA-DRB1*04:01 | 94 | 108 | YCAKVSYLSTASSLD | 1.90 |
| | | | (SEQ ID NO: 713) | |
| HLA-DRB1*04:01 | 93 | 107 | YYCAKVSYLSTASSL | 1.90 |
| | | | (SEQ ID NO: 714) | |
| HLA-DRB1*04:05 | 74 | 88 | AKNSLYLQMNSLRAE | 2.10 |
| | | | (SEQ ID NO: 715) | |
| HLA-DRB3*02:02 | 74 | 88 | AKNSLYLQMNSLRAE | 2.10 |
| | | | (SEQ ID NO: 716) | |
| HLA-DRB1*04:05 | 75 | 89 | KNSLYLQMNSLRAED | 2.10 |
| | | | (SEQ ID NO: 717) | |
| HLA-DRB3*02:02 | 75 | 89 | KNSLYLQMNSLRAED | 2.10 |
| | | | (SEQ ID NO: 718) | |
| HLA-DRB1*04:05 | 78 | 92 | LYLQMNSLRAEDTAV | 2.10 |
| | | | (SEQ ID NO: 719) | |
| HLA-DRB1*04:05 | 73 | 87 | NAKNSLYLQMNSLRA | 2.10 |
| | | | (SEQ ID NO: 720) | |
| HLA-DRB5*01:01 | 73 | 87 | NAKNSLYLQMNSLRA | 2.10 |
| | | | (SEQ ID NO: 721) | |
| HLA-DRB1*04:05 | 76 | 90 | NSLYLQMNSLRAEDT | 2.10 |
| | | | (SEQ ID NO: 722) | |
| HLA-DRB3*02:02 | 76 | 90 | NSLYLQMNSLRAEDT | 2.10 |
| | | | (SEQ ID NO: 723) | |
| HLA-DRB1*04:05 | 77 | 91 | SLYLQMNSLRAEDTA | 2.10 |
| | | | (SEQ ID NO: 724) | |
| HLA-DRB1*04:05 | 79 | 93 | YLQMNSLRAEDTAVY | 2.10 |
| | | | (SEQ ID NO: 725) | |
| HLA-DRB3*02:02 | 77 | 91 | SLYLQMNSLRAEDTA | 2.20 |
| | | | (SEQ ID NO: 726) | |
| HLA-DRB1*08:02 | 114 | 128 | TLVTVSSASTKGPSV | 2.30 |
| | | | (SEQ ID NO: 727) | |
| HLA-DRB1*04:01 | 97 | 111 | KVSYLSTASSLDYWG | 2.40 |
| | | | (SEQ ID NO: 728) | |
| HLA-DRB1*04:01 | 82 | 96 | MNSLRAEDTAVYYCA | 2.50 |
| | | | (SEQ ID NO: 729) | |
| HLA-DRB3*02:02 | 14 | 28 | PGSLRLSCAASGFTF | 2.50 |
| | | | (SEQ ID NO: 730) | |
| HLA- | 20 | 34 | SCAASGFTFDDYAMH | 2.50 |
| DQA1*01:01/DQB1*05:01 | | | (SEQ ID NO: 731) | |
| HLA-DRB1*04:01 | 92 | 106 | VYYCAKVSYLSTASS | 2.60 |
| | | | (SEQ ID NO: 732) | |
| HLA-DRB1*04:01 | 83 | 97 | NSLRAEDTAVYYCAK | 2.70 |
| | | | (SEQ ID NO: 733) | |
| HLA-DRB1*04:01 | 112 | 126 | QGTLVTVSSASTKGP | 2.70 |
| | | | (SEQ ID NO: 734) | |
| HLA-DRB1*04:01 | 111 | 125 | GQGTLVTVSSASTKG | 2.80 |
| | | | (SEQ ID NO: 735) | |
| HLA-DRB1*08:02 | 94 | 108 | YCAKVSYLSTASSLD | 2.80 |
| | | | (SEQ ID NO: 736) | |
| HLA-DRB1*08:02 | 93 | 107 | YYCAKVSYLSTASSL | 2.80 |
| | | | (SEQ ID NO: 737) | |
| HLA-DRB3*02:02 | 15 | 29 | GSLRLSCAASGFTFD | 2.90 |
| | | | (SEQ ID NO: 738) | |
| HLA-DRB1*04:01 | 81 | 95 | QMNSLRAEDTAVYYC | 2.90 |
| | | | (SEQ ID NO: 739) | |
| HLA-DRB5*01:01 | 77 | 91 | SLYLQMNSLRAEDTA | 2.90 |
| | | | (SEQ ID NO: 740) | |
| HLA-DRB1*04:01 | 113 | 127 | GTLVTVSSASTKGPS | 3.00 |
| | | | (SEQ ID NO: 741) | |
| HLA-DRB1*08:02 | 96 | 110 | AKVSYLSTASSLDYW | 3.10 |
| | | | (SEQ ID NO: 742) | |
| HLA-DRB3*02:02 | 78 | 92 | LYLQMNSLRAEDTAV | 3.20 |
| | | | (SEQ ID NO: 743) | |
| HLA- | 2 | 16 | VQLVESGGGLVQPGS | 3.20 |
| DQA1*05:01/DQB1*03:01 | | | (SEQ ID NO: 744) | |
| HLA-DRB1*08:02 | 109 | 123 | YWGQGTLVTVSSAST | 3.20 |
| | | | (SEQ ID NO: 745) | |
| HLA-DRB1*08:02 | 95 | 109 | CAKVSYLSTASSLDY | 3.30 |
| | | | (SEQ ID NO: 746) | |
| HLA-DRB1*15:01 | 73 | 87 | NAKNSLYLQMNSLRA | 3.30 |
| | | | (SEQ ID NO: 747) | |
| HLA-DRB3*02:02 | 73 | 87 | NAKNSLYLQMNSLRA | 3.30 |
| | | | (SEQ ID NO: 748) | |
| HLA- | 3 | 17 | QLVESGGGLVQPGSL | 3.30 |
| DQA1*05:01/DQB1*03:01 | | | (SEQ ID NO: 749) | |
| HLA-DRB3*02:02 | 13 | 27 | QPGSLRLSCAASGFT | 3.30 |
| | | | (SEQ ID NO: 750) | |
| HLA-DRB1*15:01 | 74 | 88 | AKNSLYLQMNSLRAE | 3.40 |
| | | | (SEQ ID NO: 751) | |
| HLA-DRB1*15:01 | 75 | 89 | KNSLYLQMNSLRAED | 3.40 |
| | | | (SEQ ID NO: 752) | |
| HLA-DRB1*15:01 | 76 | 90 | NSLYLQMNSLRAEDT | 3.40 |
| | | | (SEQ ID NO: 753) | |
| HLA- | 22 | 36 | AASGFTFDDYAMHWV | 3.70 |
| DQA1*01:01/DQB1*05:01 | | | (SEQ ID NO: 754) | |
| HLA-DRB1*04:01 | 80 | 94 | LQMNSLRAEDTAVYY | 3.70 |
| | | | (SEQ ID NO: 755) | |
| HLA- | 4 | 18 | LVESGGGLVQPGSLR | 3.70 |
| DQA1*05:01/DQB1*03:01 | | | (SEQ ID NO: 756) | |
| HLA-DRB1*04:01 | 110 | 124 | WGQGTLVTVSSASTK | 3.70 |
| | | | (SEQ ID NO: 757) | |
| HLA-DRB4*01:01 | 73 | 87 | NAKNSLYLQMNSLRA | 3.90 |
| | | | (SEQ ID NO: 758) | |
| HLA-DRB1*08:02 | 92 | 106 | VYYCAKVSYLSTASS | 3.90 |
| | | | (SEQ ID NO: 759) | |
| HLA-DRB1*04:01 | 98 | 112 | VSYLSTASSLDYWGQ | 4.10 |
| | | | (SEQ ID NO: 760) | |
| HLA-DRB1*07:01 | 96 | 110 | AKVSYLSTASSLDYW | 4.20 |
| | | | (SEQ ID NO: 761) | |
| HLA- | 23 | 37 | ASGFTFDDYAMHWVR | 4.20 |
| DQA1*01:01/DQB1*05:01 | | | (SEQ ID NO: 762) | |
| HLA- | 5 | 19 | VESGGGLVQPGSLRL | 4.30 |
| DQA1*05:01/DQB1*03:01 | | | (SEQ ID NO: 763) | |
| HLA-DRB3*01:01 | 22 | 36 | AASGFTFDDYAMHWV | 4.40 |
| | | | (SEQ ID NO: 764) | |
| HLA- | 96 | 110 | AKVSYLSTASSLDYW | 4.40 |
| DPA1*02:01/DPB1*14:01 | | | (SEQ ID NO: 765) | |
| HLA-DRB3*01:01 | 23 | 37 | ASGFTFDDYAMHWVR | 4.50 |
| | | | (SEQ ID NO: 766) | |
| HLA- | 1 | 15 | EVQLVESGGGLVQPG | 4.50 |
| DQA1*05:01/DQB1*03:01 | | | (SEQ ID NO: 767) | |
| HLA-DRB3*01:01 | 21 | 35 | CAASGFTFDDYAMHW | 4.60 |
| | | | (SEQ ID NO: 768) | |
| HLA-DRB3*01:01 | 80 | 94 | LQMNSLRAEDTAVYY | 4.60 |
| | | | (SEQ ID NO: 769) | |
| HLA- | 17 | 31 | LRLSCAASGFTFDDY | 4.60 |
| DQA1*01:01/DQB1*05:01 | | | (SEQ ID NO: 770) | |
| HLA- | 19 | 33 | LSCAASGFTFDDYAM | 4.60 |
| DQA1*01:01/DQB1*05:01 | | | (SEQ ID NO: 771) | |
| HLA-DRB3*01:01 | 82 | 96 | MNSLRAEDTAVYYCA | 4.60 |
| | | | (SEQ ID NO: 772) | |
| HLA-DRB3*01:01 | 83 | 97 | NSLRAEDTAVYYCAK | 4.60 |
| | | | (SEQ ID NO: 773) | |
| HLA-DRB3*01:01 | 81 | 95 | QMNSLRAEDTAVYYC | 4.60 |
| | | | (SEQ ID NO: 774) | |
| HLA- | 18 | 32 | RLSCAASGFTFDDYA | 4.60 |
| DQA1*01:01/DQB1*05:01 | | | (SEQ ID NO: 775) | |
| HLA-DRB4*01:01 | 74 | 88 | AKNSLYLQMNSLRAE | 4.70 |
| | | | (SEQ ID NO: 776) | |
| HLA-DRB1*04:01 | 41 | 55 | GKGLEWVSAITWNSG | 4.70 |
| | | | (SEQ ID NO: 777) | |
| HLA-DRB1*04:01 | 42 | 56 | KGLEWVSAITWNSGH | 4.70 |
| | | | (SEQ ID NO: 778) | |
| HLA-DRB4*01:01 | 75 | 89 | KNSLYLQMNSLRAED | 4.70 |
| | | | (SEQ ID NO: 779) | |
| HLA-DRB3*01:01 | 24 | 38 | SGFTFDDYAMHWVRQ | 4.70 |
| | | | (SEQ ID NO: 780) | |
| HLA-DRB1*13:02 | 46 | 60 | WVSAITWNSGHIDYA | 4.70 |
| | | | (SEQ ID NO: 781) | |
| HLA-DRB1*07:01 | 95 | 109 | CAKVSYLSTASSLDY | 4.80 |
| | | | (SEQ ID NO: 782) | |
| HLA-DRB1*01:01 | 96 | 110 | AKVSYLSTASSLDYW | 4.90 |
| | | | (SEQ ID NO: 783) | |
| HLA-DRB1*01:01 | 95 | 109 | CAKVSYLSTASSLDY | 4.90 |
| | | | (SEQ ID NO: 784) | |
| HLA-DRB3*02:02 | 65 | 79 | GRFTISRDNAKNSLY | 4.90 |
| | | | (SEQ ID NO: 785) | |
| HLA-DRB3*02:02 | 12 | 26 | VQPGSLRLSCAASGF | 4.90 |
| | | | (SEQ ID NO: 786) | |
| HLA-DRB1*01:01 | 15 | 29 | GSLRLSCAASGFTFD | 5.00 |
| | | | (SEQ ID NO: 787) | |

Accordingly, in certain embodiments the epitopes comprising the tolerogenic nanoparticles described herein comprises one or more of the epitopes shown in Table 37 and/or the corresponding nucleic acid sequences for loading into nanoparticles.

The adalimumab light chain intact protein sequence is DIQMTQSPSSLSASVGDRVTITCRASQGIRNYLAWYQQKPGKAPKLLIYAASTLQSG VPSRFSGSGSGTDFTLTISSLQPEDVATYYCQRYNRAPYTFGQGTKVEIKRTVAAPSV FIFPP (SEQ ID NO:788). This sequence was used for human MHC-II binding interactions, utilizing the full HLA reference set. We only list the top 5% of predictions based on affinity binding interactions (see Table 38).

TABLE 38
Human T cell epitope predictions for Adm light chain by the IEDB database.

| | | | | Percentile |
|---|---|---|---|---|
| Allele | Start | End | Sequence | Rank |
| HLA-DPA1*02:01/DPB1*14:01 | 103 | 117 | KVEIKRTVAAPSVFI | 0.01 |
| | | | (SEQ ID NO: 789) | |
| HLA-DPA1*02:01/DPB1*14:01 | 104 | 118 | VEIKRTVAAPSVFIF | 0.01 |
| | | | (SEQ ID NO: 790) | |
| HLA-DPA1*02:01/DPB1*14:01 | 105 | 119 | EIKRTVAAPSVFIFP | 0.03 |
| | | | (SEQ ID NO: 791) | |
| HLA-DPA1*02:01/DPB1*14:01 | 102 | 116 | TKVEIKRTVAAPSVF | 0.03 |
| | | | (SEQ ID NO: 792) | |
| HLA-DPA1*02:01/DPB1*14:01 | 106 | 120 | IKRTVAAPSVFIFPP | 0.12 |
| | | | (SEQ ID NO: 793) | |
| HLA-DPA1*02:01/DPB1*14:01 | 101 | 115 | GTKVEIKRTVAAPSV | 0.19 |
| | | | (SEQ ID NO: 794) | |
| HLA- | 45 | 59 | KLLIYAASTLQSGVP | 0.50 |
| DQA1*01:02/DQB1*06:02 | | | (SEQ ID NO: 795) | |
| HLA- | 46 | 60 | LLIYAASTLQSGVPS | 0.56 |
| DQA1*01:02/DQB1*06:02 | | | (SEQ ID NO: 796) | |
| HLA- | 44 | 58 | PKLLIYAASTLQSGV | 0.56 |
| DQA1*01:02/DQB1*06:02 | | | (SEQ ID NO: 797) | |
| HLA-DRB1*08:02 | 102 | 116 | TKVEIKRTVAAPSVF | 0.83 |
| | | | (SEQ ID NO: 798) | |
| HLA-DRB1*15:01 | 43 | 57 | APKLLIYAASTLQSG | 0.85 |
| | | | (SEQ ID NO: 799) | |
| HLA-DRB1*15:01 | 41 | 55 | GKAPKLLIYAASTLQ | 0.85 |
| | | | (SEQ ID NO: 800) | |
| HLA-DRB1*08:02 | 101 | 115 | GTKVEIKRTVAAPSV | 0.85 |
| | | | (SEQ ID NO: 801) | |
| HLA-DRB1*15:01 | 42 | 56 | KAPKLLIYAASTLQS | 0.85 |
| | | | (SEQ ID NO: 802) | |
| HLA-DRB1*15:01 | 40 | 54 | PGKAPKLLIYAASTL | 0.85 |
| | | | (SEQ ID NO: 803) | |
| HLA-DRB1*15:01 | 44 | 58 | PKLLIYAASTLQSGV | 0.85 |
| | | | (SEQ ID NO: 804) | |
| HLA-DRB1*15:01 | 23 | 37 | CRASQGIRNYLAWYQ | 1.10 |
| | | | (SEQ ID NO: 805) | |
| HLA-DRB1*08:02 | 103 | 117 | KVEIKRTVAAPSVFI | 1.10 |
| | | | (SEQ ID NO: 806) | |
| HLA- | 47 | 61 | LIYAASTLQSGVPSR | 1.10 |
| DQA1*01:02/DQB1*06:02 | | | (SEQ ID NO: 807) | |
| HLA-DRB1*08:02 | 44 | 58 | PKLLIYAASTLQSGV | 1.10 |
| | | | (SEQ ID NO: 808) | |
| HLA-DRB1*15:01 | 24 | 38 | RASQGIRNYLAWYQQ | 1.10 |
| | | | (SEQ ID NO: 809) | |
| HLA-DRB1*08:02 | 43 | 57 | APKLLIYAASTLQSG | 1.20 |
| | | | (SEQ ID NO: 810) | |
| HLA-DRB1*09:01 | 94 | 108 | APYTFGQGTKVEIKR | 1.20 |
| | | | (SEQ ID NO: 811) | |
| HLA-DRB1*09:01 | 92 | 106 | NRAPYTFGQGTKVEI | 1.20 |
| | | | (SEQ ID NO: 812) | |
| HLA-DPA1*02:01/DPB1*14:01 | 44 | 58 | PKLLIYAASTLQSGV | 1.20 |
| | | | (SEQ ID NO: 813) | |
| HLA-DRB1*09:01 | 93 | 107 | RAPYTFGQGTKVEIK | 1.20 |
| | | | (SEQ ID NO: 814) | |
| HLA-DRB1*15:01 | 25 | 39 | ASQGIRNYLAWYQQK | 1.30 |
| | | | (SEQ ID NO: 815) | |
| HLA-DRB1*08:02 | 42 | 56 | KAPKLLIYAASTLQS | 1.30 |
| | | | (SEQ ID NO: 816) | |
| HLA-DPA1*02:01/DPB1*14:01 | 45 | 59 | KLLIYAASTLQSGVP | 1.40 |
| | | | (SEQ ID NO: 817) | |
| HLA-DRB3*02:02 | 103 | 117 | KVEIKRTVAAPSVFI | 1.40 |
| | | | (SEQ ID NO: 818) | |
| HLA-DRB1*09:01 | 90 | 104 | RYNRAPYTFGQGTKV | 1.40 |
| | | | (SEQ ID NO: 819) | |
| HLA-DRB1*15:01 | 26 | 40 | SQGIRNYLAWYQQKP | 1.40 |
| | | | (SEQ ID NO: 820) | |
| HLA-DRB1*09:01 | 91 | 105 | YNRAPYTFGQGTKVE | 1.40 |
| | | | (SEQ ID NO: 821) | |
| HLA-DPA1*02:01/DPB1*14:01 | 43 | 57 | APKLLIYAASTLQSG | 1.50 |
| | | | (SEQ ID NO: 822) | |
| HLA- | 68 | 82 | GTDFTLTISSLQPED | 1.50 |
| DQA1*03:01/DQB1*03:02 | | | (SEQ ID NO: 823) | |
| HLA-DRB1*08:02 | 45 | 59 | KLLIYAASTLQSGVP | 1.50 |
| | | | (SEQ ID NO: 824) | |
| HLA-DRB1*08:02 | 100 | 114 | QGTKVEIKRTVAAPS | 1.50 |
| | | | (SEQ ID NO: 825) | |
| HLA-DPA1*02:01/DPB1*14:01 | 42 | 56 | KAPKLLIYAASTLQS | 1.60 |
| | | | (SEQ ID NO: 826) | |
| HLA-DRB1*15:01 | 27 | 41 | QGIRNYLAWYQQKPG | 1.60 |
| | | | (SEQ ID NO: 827) | |
| HLA-DRB1*04:01 | 43 | 57 | APKLLIYAASTLQSG | 1.70 |
| | | | (SEQ ID NO: 828) | |
| HLA- | 70 | 84 | DFTLTISSLQPEDVA | 1.70 |
| DQA1*03:01/DQB1*03:02 | | | (SEQ ID NO: 829) | |
| HLA-DRB1*04:01 | 44 | 58 | PKLLIYAASTLQSGV | 1.70 |
| | | | (SEQ ID NO: 830) | |
| HLA- | 69 | 83 | TDFTLTISSLQPEDV | 1.70 |
| DQA1*03:01/DQB1*03:02 | | | (SEQ ID NO: 831) | |
| HLA- | 71 | 85 | FTLTISSLQPEDVAT | 2.00 |
| DQA1*03:01/DQB1*03:02 | | | (SEQ ID NO: 832) | |
| HLA-DRB3*02:02 | 102 | 116 | TKVEIKRTVAAPSVF | 2.00 |
| | | | (SEQ ID NO: 833) | |
| HLA- | 67 | 81 | SGTDFTLTISSLQPE | 2.10 |
| DQA1*03:01/DQB1*03:02 | | | (SEQ ID NO: 834) | |
| HLA-DRB1*08:02 | 104 | 118 | VEIKRTVAAPSVFIF | 2.10 |
| | | | (SEQ ID NO: 835) | |
| HLA-DRB3*02:02 | 104 | 118 | VEIKRTVAAPSVFIF | 2.10 |
| | | | (SEQ ID NO: 836) | |
| HLA-DRB1*08:02 | 46 | 60 | LLIYAASTLQSGVPS | 2.20 |
| | | | (SEQ ID NO: 837) | |
| HLA- | 43 | 57 | APKLLIYAASTLQSG | 2.30 |
| DQA1*01:02/DQB1*06:02 | | | (SEQ ID NO: 838) | |
| HLA- | 68 | 82 | GTDFTLTISSLQPED | 2.30 |
| DQA1*04:01/DQB1*04:02 | | | (SEQ ID NO: 839) | |
| HLA-DRB1*04:05 | 68 | 82 | GTDFTLTISSLQPED | 2.30 |
| | | | (SEQ ID NO: 840) | |
| HLA- | 48 | 62 | IYAASTLQSGVPSRF | 2.30 |
| DQA1*01:02/DQB1*06:02 | | | (SEQ ID NO: 841) | |
| HLA- | 42 | 56 | KAPKLLIYAASTLQS | 2.30 |
| DQA1*01:02/DQB1*06:02 | | | (SEQ ID NO: 842) | |
| HLA-DRB1*04:01 | 45 | 59 | KLLIYAASTLQSGVP | 2.30 |
| | | | (SEQ ID NO: 843) | |
| HLA-DRB1*04:01 | 103 | 117 | KVEIKRTVAAPSVFI | 2.40 |
| | | | (SEQ ID NO: 844) | |
| HLA-DRB1*04:05 | 69 | 83 | TDFTLTISSLQPEDV | 2.50 |
| | | | (SEQ ID NO: 845) | |
| HLA-DRB1*04:01 | 102 | 116 | TKVEIKRTVAAPSVF | 2.50 |
| | | | (SEQ ID NO: 846) | |
| HLA-DRB1*01:01 | 42 | 56 | KAPKLLIYAASTLQS | 2.60 |
| | | | (SEQ ID NO: 847) | |
| HLA-DPA1*02:01/DPB1*14:01 | 100 | 114 | QGTKVEIKRTVAAPS | 2.60 |
| | | | (SEQ ID NO: 848) | |
| HLA-DRB1*01:01 | 43 | 57 | APKLLIYAASTLQSG | 2.90 |
| | | | (SEQ ID NO: 849) | |
| HLA-DRB1*01:01 | 41 | 55 | GKAPKLLIYAASTLQ | 2.90 |
| | | | (SEQ ID NO: 850) | |
| HLA-DRB1*15:01 | 45 | 59 | KLLIYAASTLQSGVP | 2.90 |
| | | | (SEQ ID NO: 851) | |
| HLA- | 27 | 41 | QGIRNYLAWYQQKPG | 2.90 |
| DQA1*01:01/DQB1*05:01 | | | (SEQ ID NO: 852) | |
| HLA-DRB1*04:05 | 67 | 81 | SGTDFTLTISSLQPE | 3.00 |
| | | | (SEQ ID NO: 853) | |
| HLA-DRB1*04:01 | 104 | 118 | VEIKRTVAAPSVFIF | 3.00 |
| | | | (SEQ ID NO: 854) | |
| HLA-DRB1*01:01 | 44 | 58 | PKLLIYAASTLQSGV | 3.10 |
| | | | (SEQ ID NO: 855) | |
| HLA- | 26 | 40 | SQGIRNYLAWYQQKP | 3.10 |
| DQA1*01:01/DQB1*05:01 | | | (SEQ ID NO: 856) | |
| HLA-DRB3*02:02 | 101 | 115 | GTKVEIKRTVAAPSV | 3.50 |
| | | | (SEQ ID NO: 857) | |
| HLA- | 67 | 81 | SGTDFTLTISSLQPE | 3.50 |
| DQA1*04:01/DQB1*04:02 | | | (SEQ ID NO: 858) | |
| HLA-DRB3*02:02 | 105 | 119 | EIKRTVAAPSVFIFP | 3.60 |
| | | | (SEQ ID NO: 859) | |
| HLA-DRB1*04:01 | 101 | 115 | GTKVEIKRTVAAPSV | 3.60 |
| | | | (SEQ ID NO: 860) | |
| HLA- | 28 | 42 | GIRNYLAWYQQKPGK | 3.90 |
| DQA1*01:01/DQB1*05:01 | | | (SEQ ID NO: 861) | |
| HLA-DRB1*08:02 | 28 | 42 | GIRNYLAWYQQKPGK | 4.00 |
| | | | (SEQ ID NO: 862) | |
| HLA-DRB1*09:01 | 103 | 117 | KVEIKRTVAAPSVFI | 4.00 |
| | | | (SEQ ID NO: 863) | |
| HLA-DRB1*09:01 | 104 | 118 | VEIKRTVAAPSVFIF | 4.00 |
| | | | (SEQ ID NO: 864) | |
| HLA-DRB1*04:01 | 42 | 56 | KAPKLLIYAASTLQS | 4.20 |
| | | | (SEQ ID NO: 865) | |
| HLA-DRB1*04:01 | 47 | 61 | LIYAASTLQSGVPSR | 4.20 |
| | | | (SEQ ID NO: 866) | |
| HLA-DRB1*04:01 | 46 | 60 | LLIYAASTLQSGVPS | 4.20 |
| | | | (SEQ ID NO: 867) | |
| HLA-DRB1*08:02 | 27 | 41 | QGIRNYLAWYQQKPG | 4.30 |
| | | | (SEQ ID NO: 868) | |
| HLA-DPA1*02:01/DPB1*14:01 | 41 | 55 | GKAPKLLIYAASTLQ | 4.40 |
| | | | (SEQ ID NO: 869) | |
| HLA-DRB3*02:02 | 44 | 58 | PKLLIYAASTLQSGV | 4.40 |
| | | | (SEQ ID NO: 870) | |
| HLA-DRB1*07:01 | 105 | 119 | EIKRTVAAPSVFIFP | 4.50 |
| | | | (SEQ ID NO: 871) | |
| HLA-DRB1*07:01 | 106 | 120 | IKRTVAAPSVFIFPP | 4.50 |
| | | | (SEQ ID NO: 872) | |
| HLA-DRB1*07:01 | 103 | 117 | KVEIKRTVAAPSVFI | 4.50 |
| | | | (SEQ ID NO: 873) | |
| HLA- | 69 | 83 | TDFTLTISSLQPEDV | 4.50 |
| DQA1*04:01/DQB1*04:02 | | | (SEQ ID NO: 874) | |
| HLA-DRB1*07:01 | 102 | 116 | TKVEIKRTVAAPSVF | 4.50 |
| | | | (SEQ ID NO: 875) | |
| HLA-DRB1*07:01 | 104 | 118 | VEIKRTVAAPSVFIF | 4.50 |
| | | | (SEQ ID NO: 876) | |
| HLA-DRB1*01:01 | 40 | 54 | PGKAPKLLIYAASTL | 4.60 |
| | | | (SEQ ID NO: 877) | |
| HLA-DRB1*04:05 | 43 | 57 | APKLLIYAASTLQSG | 4.70 |
| | | | (SEQ ID NO: 878) | |
| HLA-DRB3*02:02 | 45 | 59 | KLLIYAASTLQSGVP | 4.70 |
| | | | (SEQ ID NO: 879) | |
| HLA- | 29 | 43 | IRNYLAWYQQKPGKA | 4.80 |
| DQA1*01:01/DQB1*05:01 | | | (SEQ ID NO: 880) | |
| HLA-DRB1*15:01 | 46 | 60 | LLIYAASTLQSGVPS | 4.80 |
| | | | (SEQ ID NO: 881) | |
| HLA-DRB1*07:01 | 101 | 115 | GTKVEIKRTVAAPSV | 4.90 |
| | | | (SEQ ID NO: 882) | |
| HLA-DRB1*01:01 | 45 | 59 | KLLIYAASTLQSGVP | 4.90 |
| | | | (SEQ ID NO: 883) | |
| HLA-DRB1*04:05 | 44 | 58 | PKLLIYAASTLQSGV | 4.90 |
| | | | (SEQ ID NO: 884) | |
| HLA- | 70 | 84 | DFTLTISSLQPEDVA | 5.00 |
| DQA1*04:01/DQB1*04:02 | | | (SEQ ID NO: 885) | |

Accordingly, in certain embodiments the epitopes comprising the tolerogenic nanoparticles described herein comprises one or more of the epitopes shown in Table 38 and/or the corresponding nucleic acid sequences for nanoparticle loading.

Comparison of the literature with the IEDB predictions confirmed the overlapping sequences, including the demonstration of their location in H and L chain CDR regions. Table 39 shows the location of the overlapping T cell epitope regions (red) in relation to the CDRs (highlighted in green).

TABLE 39
Localization of human T cell epitopes that are present on adalimumab heavy and
light chains. Extensive overlap in the regions identified by Meunier et al., (2020)
Cell Mol Immunol. 17: 656-658), and the IEDB search, which we propose can serve as
human T cell epitopes (red) for tolerogenic therapy. The location of the T cell
epitopes (red) are compared to CDR domains (highlighted in green).

Adalimumab heavy chain with T cell epitopes highlighted in red (SEQ ID NO: 886)
EVQLVESGGGLVQPGSLRLSCAASGFTFDDYAMHWVRQAPGKGLEWVSAIT
WNSGHIDYADSVEGRFTISRDNAKNSLYLQMNSLRAEDTAVYYCAKVSYLSTAS
SLDYWGQGTLVTVSSASTKGPSVF
Adalimumab heavy chain with T cell epitopes highlighted in red (SEQ ID NO: 887)
DIQMTQSPSSLSASVGDRVTITCRASQGIRNYLAWYQQKPGKAPKLLIYAASTL
QSGVPSRFSGSGSGTDFTLTISSSLQPEDVATYYCQRYNRAPYRFGQGTKVEIKR
TVAAPSVFIFPP

The peptide sequences of the T-cell epitopes identified in Table 39 are shown in Table 40.

TABLE 40
Adalimumab heavy and light chain T cell epitopes that overlap with CDRs.

| | | SEQ |
|---|---|---|
| Region | Sequence | ID NO |
| Heavy chain | EWVSAITWNSGHIDYADSVE | 888 |
| Heavy chain | LQMNSLRAEDTAVYYCAKVSYLSTASSLDYWGQGTLVT | 889 |
| | VS | |
| Light chain | TCRASQGIRNYLAWYQQKPGKAPKLLIYAASTLQ | 890 |
| Light chain | EDVATYYCQRYNRAPYTFGQGTKVEIKRTVAAPSV | 891 |

Accordingly, in certain embodiments the epitopes comprising the tolerogenic nanoparticles described herein comprises one or more of the epitopes shown in Table 40, as well as their corresponding nucleic acid sequences.

Considering the above data set, we have formulated a short list of T-cell epitopes for the adalimumab heavy and light chain to be used as lead sequences for peptide and mRNA vaccine design (Tables 41 and 42).

TABLE 41
Adalimumab heavy chain human T cell epitopes (best loading sequences in bold)

| No. | Epitope | Location | Sequence | pI | GRAVY |
|---|---|---|---|---|---|
| 1 | Epitope 1 | 45-64 | EWVSAITWNSGHIDYADSVE | 3.91 | −0.315 |
| | | | (SEQ ID NO: 892) | | |
| 2 | Epitope 2 | 80-99- | LQMNSLRAEDTAVYYCAKVS | 6.06 | −0.075 |
| | | | (SEQ ID NO: 893) | | |
| 3 | Epitope 3 | 90-109 | TAVYYCAKVSYLSTASSLDY | 5.50 | 0.335 |
| | | | (SEQ ID NO: 894) | | |
| 4 | Epitope 4 | 100-119 | YLSTASSLDYWGQGTLVTVS | 3.80 | 0.250 |
| | | | (SEQ ID NO: 895) | | |

TABLE 42
Adalimumab light chain human T cell epitopes (best loading sequences in bold)

| No. | Epitope | Specifications | Sequence | pI | GRAVY |
|---|---|---|---|---|---|
| 1 | Epitope 1 | 21-40 | ITCRASQGIRNYLAWYQQKP | 9.78 | −0.750 |
| | | | (SEQ ID NO: 896) | | |
| 2 | Epitope 2 | 36-55- | YQQKPGKAPKLLIYAASTLQ | 9.83 | −0.430 |
| | | | (SEQ ID NO: 897) | | |
| 3 | Epitope 3 | 81-100 | EDVATYYCQRYNRAPYTFGQ | 6.15 | −1.100 |
| | | | (SEQ ID NO: 898) | | |
| 4 | Epitope 4 | 96-115 | YTFGQGTKVEIKRTVAAPSV | 9.70 | −0.120 |
| | | | (SEQ ID NO: 899) | | |

Accordingly, in certain embodiments the epitopes comprising the tolerogenic nanoparticles described herein comprises one or more of the epitopes shown in Tables 41 and/or 42 and/or the corresponding nucleic acid sequences that can be used for particle loading

Utilizing the p1 and GRAVY calculations, Tables 41 and 42 predicts the epitopes (in bold) most suitable for particle loading based on solubility and charge predictions. These peptide sequences could be used individually or in combination for PLGA nanoparticles loading. Another consideration would be multi-epitope mRNA vaccine design for nanoparticle development, as described herein.

Similar to the integrated nucleotide design process described for peanut allergens, we will use reverse translation of H and L-chain epitopes (Table 43), linker addition, insertion of an upstream a Trf1-118 domain, plusadding start and stop codons to develop a tolerogenic mRNA “vaccine” for human use. The coding sequence will be inserted into a pTNT plasmid that will be used, to synthesize a multi-epitope mRNA strand for nanoparticle encapsulation.

TABLE 43
Reverse translation of Adalimumab T cell epitopes for therapeutic studies

| No. | Epitope | Location | Reverse translated cDNA sequence |
|---|---|---|---|
| 1 | HC #1 | 45-64 | gaatgggtgagcgcgattacctggaacagcggccatattgattatgcggatagcgtggaa |
| | | | (SEQ ID NO: 900) |
| 2 | HC #2 | 80-99 | ctgcagatgaacagcctgcgcgcggaagataccgcggtgtattattgcgcgaaagtgagc |
| | | | (SEQ ID NO: 901) |
| 3 | HC #3 | 90-109 | accgcggtgtattattgcgcgaaagtgagctatctgagcaccgcgagcagcctggattat |
| | | | (SEQ ID NO: 902) |
| 4 | HC #4 | 100-119 | tatctgagcaccgcgagcagcctggattattggggccagggcaccctggtgaccgtgagc |
| | | | (SEQ ID NO: 903) |
| 5 | LC #1 | 21-40 | attacctgccgcgcgagccagggcattcgcaactatctggcgtggtatcagcagaaaccg |
| | | | (SEQ ID NO: 904) |
| 6 | LC #2 | 36-55 | tatcagcagaaaccgggcaaagcgccgaaactgctgatttatgcggcgagcaccctgcag |
| | | | (SEQ ID NO: 905) |
| 7 | LC #3 | 81-100 | gaagatgtggcgacctattattgccagcgctataaccgcgcgccgtatacctttggccag |
| | | | (SEQ ID NO: 906) |
| 8 | LC #4 | 96-115 | tatacctttggccagggcaccaaagtggaaattaaacgcaccgtggcggcgccgagcgtg |
| | | | (SEQ ID NO: 907) |

Table 44 illustrates final codon-optimized coding sequence for adalimumab t cell epitopes

TABLE 44
Codon-optimized sequence for multi-epitope vaccine
delivery to obtain immune tolerization against
adalimumab ADA. Complete coding sequence with
human TfR1-118 domain (cyan), spliced in upstream
of the selected T cell epitopes, containing
cleavable linkers (yellow and green). After the
addition of start (purple) and stop codons (red),
codon optimization was performed, showing an
average GC content of 63.30%.

ATGATGGACCAGGCCAGAAGCGCCTTCAGCAACCTGTTCGGCGGCGAGCC
CCTGAGCTACACCAGATTCAGCCTGGCAAGACAGGTGGACGGCGACAACA
GCCACGTGGAAATGAAACTGGCCGTGGACGAGGAAGAAAACGCCGATAA
CAATACCAAGGCTAATGTGACAAAGCCCAAGAGATGCAGCGGCAGCATCT
GCTACGGCACCATCGCCGTGATCGTGTTCTTCCTGATCGGCTTCATGATC
GGATATCTGGGCTACTGCAAGGGCGTGGAACCTAAGACCGAGTGCGAGA
GACTGGCCGGCACAGAGAGCCCTGTGAGGGAAGAGCCAGGAGAAGATTT
CCCCGCTGGCGGCGGAGGTAGCAGAGTGAGACGGGGCGGCGGAGGAAGT
CAGGAGTGGGTGTCCGCCATCACATGGAACAGCGGCCACATCGACTACGC
CGACAGCGTGGAAGGCGGCGGAGGCAGCCGGGTCCGGAGAGGCGGCGG
AGGCTCTCTCCAGATGAACTCTCTGAGAGCCGAGGACACCGCCGTGTACT
ACTGCGCCAAGGTGAGCGGCGGCGGCGGCAGCAGAGTTAGACGGGGCGG
CGGCGGCTCTACAGCCGTGTACTACTGTGCCAAGGTGTCCTATCTGTCTA
CCGCCTCTAGCCTGGACTACGGAGGCGGCGGTAGCAGAGTCAGAAGAGG
AGGCGGCGGAAGCTACCTGAGCACCGCCAGCAGCCTGGATTACTGGGGC
CAGGGAACACTGGTGACCGTGTCTGGAGGCGGAGGATCCAGAGTGCGGC
GGGGAGGCGGCGGTTCCATCACCTGCAGAGCCAGCCAGGGCATTAGAAA
CTACCTGGCCTGGTACCAGCAGAAGCCTGGCGGCGGCGGCTCTAGAGTGC
GCCGCGGCGGCGGCGGCAGCTACCAACAGAAACCTGGCAAGGCCCCAAA
ACTGCTGATCTACGCTGCTTCTACCCTGCAGGGCGGCGGGGGCAGCAGAG
TGAGACGGGGCGGCGGCGGCAGCGAGGATGTGGCTACATACTACTGTCA
GAGATACAACCGGGCCCCTTACACTTTTGGCCAGGGCGGCGGCGGCAGCA
GAGTGCGTAGAGGCGGAGGCGGCAGCTATACCTTTGGCCAAGGTACCAA
GGTCGAGATCAAGCGGACCGTGGCCGCCCCTAGCGTGCGGGTTCGGAGA
TGA (SEQ ID NO: 908)

### Diabetes.

Type 1 diabetes (TID) is caused by diabetogenic T-cells, which have escaped central and peripheral tolerance and are capable of destroying insulin-producing β-cells through epitope recognition of islet cell autoantigens. Targeting these CD4+ and CD8+T-cells for deletion, inactivation or attaining a protective phenotype, without disrupting the rest of the immune system, has been the goal of antigen-specific therapies for TID. Multiple β-cell antigens may be involved, among which insulin (INS), glutamic acid decarboxylase 65 (GAD65), and insulinoma-associated protein 2 (IA-2) featured early on as the best-studied targets, reflected by the number of literature reports and number of epitopes (James et al. (2020) Diabetes, 69:1311-1335; Smith & Peakman (2018) Front. Immunol. 9:392). However, additional antigens of interest have emerged over time, including ZnT8 and IGRP, as well as number of post-translationally modified epitopes. Several lines of evidence support the importance of CD4 T-cells in disease pathogenesis, including the strong genetic association of TID with specific HLA class II haplotypes. In addition, CD4 T cells play an important role in the appearance of multiple islet-specific autoantibodies, which develop in advance of disease onset. These antibody responses reflect the important role of specific beta-cell antigens in the early autoimmune disease process, which often evolves by a process of epitope spreading to include additional antigens (Di Lorenzo et al. (2007) Clin. & Exp, Immunol. 148:1-16). In addition to playing a role in the generation of autoimmunity, antigen presenting CD4⁺ T-cells pave the way for interfering in TID by generating regulatory T-cell responses. This is in line with our goal of using epitope-encapsulating tolerogenic nanoparticles for targeted delivery to liver sinusoidal endothelial cells, capable of epitope presentation to naïve T-cells via MHC-II alleles. The natural ability of these APC to induce robust tolerogenic effects in animals and humans provide a strong incentive for the performance of preclinical studies that can be further pursued by clinical trials in humans.

A comprehensive number of studies have used an array of methods and approaches to identify CD4 T-cell epitopes derived in diabetes-associated autoantigens, with a view to therapeutic use (see, e.g., James et al. (2020) Diabetes, 69:1311-1335; Mannering et al. (2019) Diabetologia, 62:351-356; Smith & Peakman (2018) Front. Immunol. 9:392). While our attempts at refining the epitope selections for peptide and nucleic acid therapy are delineated below, it is important to list the key questions regarding tolerogenic therapy for TID, namely: (i) choice of the islet cell antigens to include because of genetic heterogeneity in TID patients, including a major contribution by HLA alleles; (ii) the genetic heterogeneity constitutes the basis for a precision medicine approach; (iii) disease prevention versus maintenance of beta-cell mass, reflecting the role that diverse islet cell antigens may play in involved in early versus later onset disease responses; (iv) the question as to the number of antigens or epitopes that needs to be included for comprehensive disease suppression at various disease development stages. While human studies addressing tolerogenic peptide immunotherapy will be addressed later, animal studies in autoimmune diabetes models have shown the possibility of impacting autoimmune islet cell destruction in the pancreas, including by single or multiple antigen therapy, (that could involve peptides or nucleic acids). For example, Dastagir et al. (2017) Mol. Therapy: Methods & Clinical Development, 4:27-38, recently demonstrated the ability of a DNA construct, expressing multiple β-cell antigens and/or epitopes, to modulate CD8⁺ and CD4⁺ T-cell function by engaging different classes of APC. The study showed that objective antigen presentation to CD4⁺ T-cells can be accomplished through MHC-II targeting of the diabetogenic epitope or mimotope sequences. MHC-II targeting could be accomplished by using TfR and li-chain targeting sequences, spliced in upstream of the antigens, or antigenic epitopes (Postigo-Fernandez & Creusot (2019) J. Autoimmunity, 98:13-23). Moreover, the same group (Firdessa-Fite & Creusot (2020) Molecular Therapy: Methods & Clinical Development, 16:50-62) demonstrated successful generation of tolerogenic immune responses by using a lipid-based carrier (jetMESSENGER) for delivering mRNA constructs to endogenous APC or injecting ex vivo primed dendritic cells into the peritoneal cavity. The data further showthat increasing the number of antigens can broaden the tolerogenic response compared to a single antigen. More specifically, the study by Postigo-Fernandez & Creusot (2019) J. Autoimmunity, 98:13-23) demonstrated that continuous treatment of NOD animals at later disease stage (10 weeks of age) could significantly delay the diabetic process, with relapse upon treatment discontinuation. Delivery of 4 antigens (insulin; chromogranin A; glutamate decarboxylase 65, and islet-specific glucose-6-phosphatase catalytic subunit-related protein (IGRP]) provided better disease protection than a single antigen. In contrast, early-stage disease prevention (4-6 weeks of age), using a single mimotope, had a more sustained effect. By implication, the study suggests multi-epitope therapy, which can be custom-designed according to HLA makeup, rendering the treatment more beneficial to human subjects. The use of nucleic acid technology, would also allow targeting of different APC and lymphocyte response pathways, depending on the delivery vehicle and route of administration. This provides a strong justification for multi-epitope therapy based on the use of a nanoparticle mRNA vaccine for TID.

In spite of the partial success achieved in the NOD model by Cruesot et al, the mechanism of action of tolerogenic nucleic acid therapy remains to be clarified. In theCruesot et al. study discussed above, antigen-specific FoxP3+T-cells only showed up after the administration of several doses. Moreover, of Tregs were distributed to peripheral lymph nodes in proximity to the injection site instead of appearing at the disease site (Postigo-Fernandez & Creusot, supra.). The durability of this response was also in question. Importantly, Creusot et al. (2019) J. Autoimmunity, 98:13-23) entertained the idea that the type of Treg response could differ if the plasmid DNA-encoded antigens are administered at other injection sites. In this regard, it has been demonstrated that intradermal injection or oral delivery of a GAD65-encoding plasmid DNA was superior to intramuscular injection in suppressing TID in the NOD model (Li et al. (2003) DNA Cell Biol. 2003; 22:227-232). This strengthens our notion that targeting of LSECs can be used as a natural source of APC that has a robust effect on Treg generation in the liver. The targeting of LSECs also eliminates the guesswork of what the contribution of heterogeneous APC populations could be in terms of the response outcomes by particles administered at a variety of treatment sites, where heterogeneous APC populations could play diagonally opposite roles at different disease stages.

Antigen-specific therapy to reinstate tolerance or to maintain long-term preservation of islet function has shown little success in TID patients during the use of oral insulin (Michels & Gottlieb (2018) Diabetes, 67:1211-1215) or GAD65 (Ludvigsson et al. (21012) N. Engl. J. Med. 366:433-44) administration. However, the use of plasmid DNA for the expression of proinsulin has shown transient protection of C-peptide production in a human clinical trial, without impacting long-lasting tolerance (Roep et al. (2013) Sci. Transl. Med, 5 (191): 191ra82). While there are a number of reasons for the difficulty of translating therapeutic response outcomes in mice to humans, the use of different routes of administration in animal vs human studies could constitute a reason for different response outcomes based on diverse APC populations. Other possible explanations include more heterogeneity among human HLA alleles than is the case for more restricted MHC-II diversity in inbred mice. Thus, our choice of using nanoparticles to encapsulate multiple antigens or epitopes that are selected for MHC-II presentation by LSECs, is how we propose to improve tolerogenic therapy in TID.

There have been a number of reports regarding the induction of TID tolerance in the NOD mouse model through delivery of autoantigens, including intact insulin and GAD65 proteins (e.g., Clemente-Casares et al. (2012) Cold Spring Harb. Perspect. Med. 3: a007773). We therefore used the NOD/SCID model to investigate nanoparticle efficacy in this disease model.

The following data were collected in collaboration with Dr. Manish Butte in the Division of Pediatric Immunology, and Rheumatology at UCLA. Nanoparticle synthesis, epitope encapsulation and carrier characterization was carried out by Dr. Qi Liu in the laboratory of Drs Nel and Xia, while the animal studies were carried out in the laboratory of Dr. Butte.

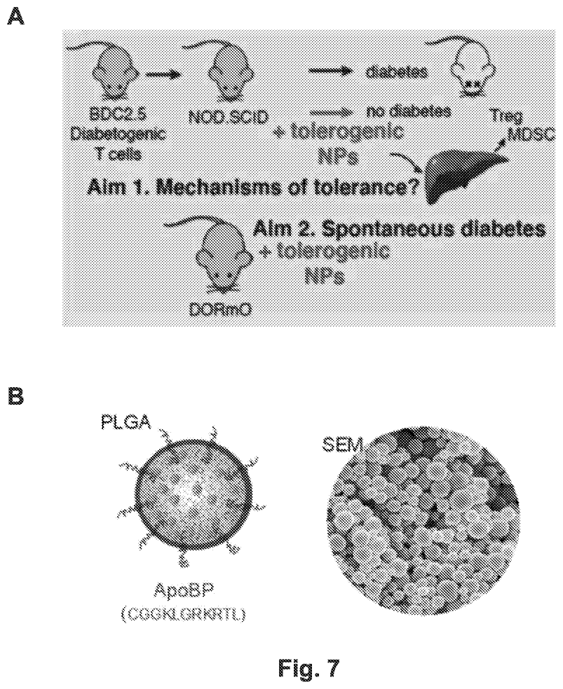

As illustrated in FIG. 7, we employed a NOD/SCID diabetes model in which diabetogenic BDC2.5 CD4 T cells from a transgenic animal were activated in vitro for 4 days with anti-CD3/28 mAb. NOC/SCID mice were injected with 200,000 activated BDC2.5 T cells at the same time as the IV administration of PLGA nanoparticles, loaded with a control (scrambled) peptide or a peptide mimotope that is recognized by the transgenic TCR. PLGA nanoparticles were synthesized by a double emulsion method to incorporate the BDC2.5 1040-51-mimotope (RTRPLWVRME (SEQ ID NO:909)), as described for peanut allergen peptides. The mimotope was purchased from Genscrpit (www.genscript.com/peptide/RP20473-BDC2_5_mimotope_1040_51.html). The synthesized particles were approximately 200 nm in diameter and encapsulated 32 μg peptide per mg PLGA. The particle surfaces were decorated with the apoB-100 peptide as previously described and IV administered to NOD/SCID mice (n=5) at various TNP doses, simultaneous with the adoptively transferred T cells. Blood glucose was monitored and mice were sacrificed after two consecutive readings, showing glucose concentrations >250 mg/dL. After sacrifice, pancreats and spleens were harvested, and cells were stained for T-cell and exhaustion markers.

### Results:

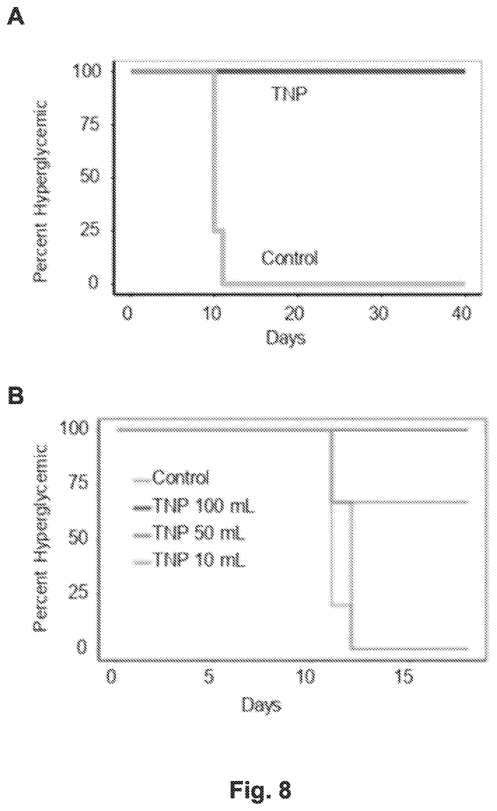

FIG. 8, panel A, shows that TNP injection into the recipient, at the same time as effector T-cells transfer, could abrogate the induction of diabetes. None of the five animals receiving the full dose (0.5 mg TNP/mouse) developed diabetes. Mice receiving the control peptide were not protected and developed rapid onset of diabetes, leading to their sacrifice. A second experiment demonstrated that the effect was dose-dependent, with mice receiving the highest dose showing 100% protection, while mice receiving 50% of the dose only showed 60% protection, while animals animals receiving 10% of the dose showing no protection (FIG. 8, panel B). F

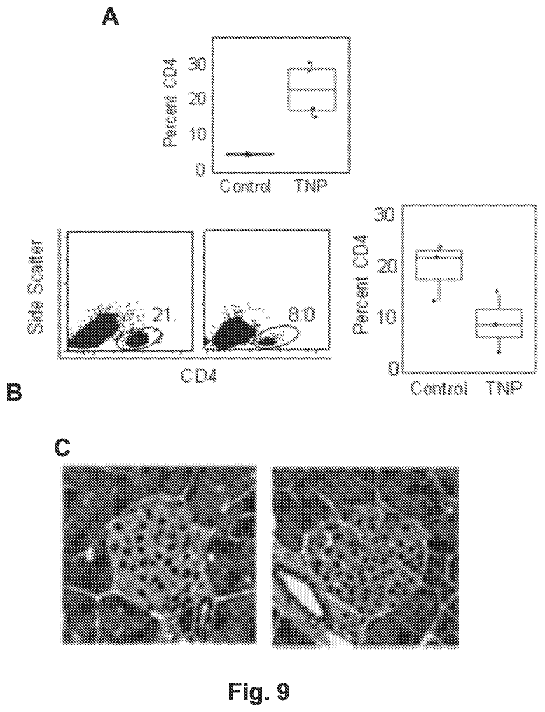

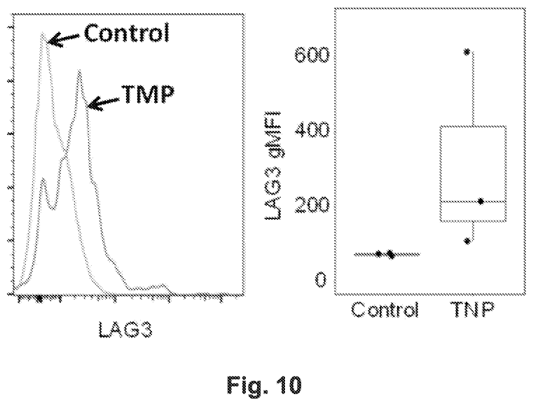

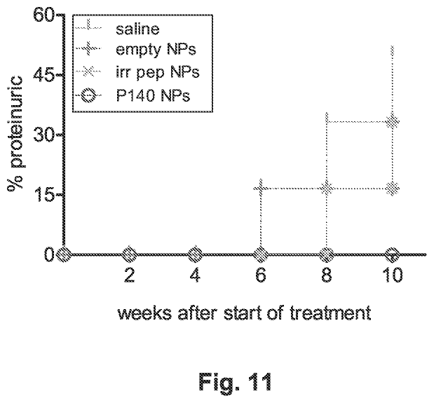

Immunohistochemistry staining of the pancreata was undertaken to investigate the hypothesis that TNP treatment would result in decreased islet damage by the transgenic T cells. The staining demonstrated reduced, but not absent infiltration of T cells in the pancreata of TNP-treated mice (FIG. 9). Instead, a sizable proportion of the adoptively transferred CD4⁺ T-cells could be seen to accumulate in the spleen. No protective effect was seen in animals treated with control nanoparticles. These data indicate that while some islet-specific diabetogenic T-cells in TNP treated mice still reaches the pancreas, this was not associated with islet cell damage or hyperglycemia. These results suggest that transferred T-cells are rendered non-pathogenic by TNP treatment. In order to see if infiltrating T cells develop inhibitory or exhaustion markers, the pancreata were also stained for CD25, PD-1, TIM3, and LAG3 markers (FIG. 10). While not observing an increase in CD25, PD-1, or TIM3, there was a sizable increase in the inhibitory LAG3 receptor on T cells in TNP-treated mice (FIG. 10). LAG3 expression is protective in autoimmune diabetes (see, e.g., Bettini et al. (2011) J. Immunol. 187 (7): 3493-3498).

In summary, our data demonstrate effective interference in islet cell damage and hyperglycemia in the NOD/SCID model by targeted mimotope delivery to LSECs. While we were able to demonstrate that activation of BDC2.5 cells by anti-CD3/CD28 is capable of generating 7.4% CD4⁺/Foxp3⁺ T-cells in the adoptively transferred population, it was not possible to accurately assess Treg abundance after adoptive transfer to NOD/SCID animals. However, we did obtain evidence of altered trafficking of diabetogenic T-cells to the spleen, in addition to ac the appearance of an exhaustion marker on these cells (FIG. 9)

Our observations are compatible to the results of Prasad, et al. (2018) J. Autoimmun. 89:112-124), who used non-targeted, biodegradable PLGA nanoparticles to administer a p31 mimotope to NOD/SCID mice (simultaneous with pre-activated BDC-2.5 transgenic T-cells). While 100% of recipients developed diabetes within 10 days, IV administration of the p31-loaded PLGA nanoparticles induced tolerogenic effects that suppressed the onset of diabetes for up to two months (Id.). The study further demonstrated a decrease in the number of effector T cells at the disease site, along with the appearance of FOXP3+ regulatory T cells (Tregs) in pancreata and spleens. Moreover, antigen-specific tolerance was demonstrated to be partially dependent on negative signaling by CTLA4 and PD-1 receptors. In a follow-up study, antigen delivery was changed to making use of a hybrid insulin peptide (HIP2.5), which confirmed that the nanoparticles exert dual effects in preventing diabetes, namely: (i) promoting T cell exhaustion (e.g., LAG3 and PD-1); (ii) and recruitment of regulatory T-cells (DiLisio & Haskins (2021) Biomedicines, 9:240). HIP2.5 is comprised of a C-peptide domain linked to a natural cleavage product of chromogranin A (ChgA). T-cells reactive to hybrid insulin peptides have also been detected in the islets of deceased human TID donors, as well as the peripheral blood of patients with new-onset TID (Baker et al. (2019) Diabetes, 68:1830-1840).

Here we demonstrate the design of single and multi-epitope mRNA constructs for experimentation in NOD and other TID animal models, with a view to collecting proof-of-principle demonstration that nucleic acid vaccines, targeted to LSECs, can be used to treat TID in a mouse. The mRNA encoding sequences will be designed to deliver the BDC2.5 Mimotope (RVLPLWVRME (SEQ ID NO:910)), the HIP2.5 peptide (LQTLALWSRMD (SEQ ID NO:911)), as well as other islet cell epitopes.

Table 45 summarizes all the epitopes that have been shown to be effective in diabetes treatment in NOD animals, including studies performed by Creusot et al. (i.e., epitopes #1-5, 7, 9 and 10).

TABLE 45
T cell epitopes for tolerance inducing studies in T1D in mice.

| No. | Epitope | Specifications | Sequence | pI | GRAVY |
|---|---|---|---|---|---|
| 1 | InsB | 9-23 | SHLVERLYLVCGERG | 6.47 | 0.013 |
| | | | (SEQ ID NO: 912) | | |
| 2 | GAD65 | 286-300 | KKGAAALGIGTDSVI | 8.59 | 0.560 |
| | | | (SEQ ID NO: 913) | | |
| 3 | InsB | 9-23 R22E | SHLVERLYLVCGEEG | 4.75 | 0.080 |
| | | | (SEQ ID NO: 914) | | |
| 4 | InsB | 9-23 E21G/R22E | SHLVERLYLVCGGEG | 5.38 | 0.287 |
| | | | (SEQ ID NO: 915) | | |
| 5 | mimotope | 2.5mi [p79] | AVPPLWVRME | 6.05 | 0.380 |
| | | | (SEQ ID NO: 916) | | |
| 6 | mimotope | BDC2.5 | RVLPLWVRME | 9.60 | 0.290 |
| | | | (SEQ ID NO: 917) | | |
| 7 | ChgA | 358-371 | WSRMDQLAKELTAE | 4.68 | −0.836 |
| | | | (SEQ ID NO: 918) | | |
| 8 | Hip2.5 | InsC + ChgA | LQTLALWSRMD | 5.84 | 0.109 |
| | | | (SEQ ID NO: 919) | | |
| 9 | InsB | 15-23 | LYLVCGERG | 5.99 | 0.467 |
| | | | (SEQ ID NO: 920) | | |
| 10 | IGRP | 206-214 | VYLKTNVFL | 9.70 | −0.389 |
| | | | (SEQ ID NO: 921) | | |

Accordingly, in certain embodiments the epitopes to be encapsulated in the tolerogenic nanoparticles described herein comprises one or more of the epitopes shown in Table 45 and/or the corresponding nucleic acid sequences.

TABLE 46
Codon-optimized single epitope (BDC2.5) vaccine
for murine use. This shows the complete coding
sequence with the mouse TfR1-118 domain (cyan),
spliced in upstream of the BDC2.5 mimotope,
embedded in cleavable linkers (yellow and
green). After the addition of start (purple)
and stop codons (red), codon optimization was
performed, rendering a construct with an average
GC content of 52.70%.

ATGATGATGGACCAGGCCCGAAGTGCCTTCAGTAATCTATTTGGGGGAGA
GCCTCTTTCCTACACACGCTTCTCCCTCGCCAGGCAGGTGGATGGAGACA
ATTCTCACGTGGAGATGAAGCTGGCTGCAGATGAAGAAGAGAATGCTGAC
AACAACATGAAAGCAAGCGTCAGGAAACCCAAGAGATTCAACGGCCGACT
GTGCTTTGCAGCCATTGCTTTAGTCATCTTTTTTTTGATAGGCTTCATGTC
TGGCTATCTTGGGTACTGCAAGCGTGTTGAGCAGAAGGAGGAATGTGTGA
AACTCGCGGAAACTGAGGAAACCGATAAGTCAGAGACCATGGAGACAGAG
GACGTGGGGGGAGGCAGCCGCGTTCGGAGAGGTGGAGGTGGCTCGCAAC
GGGTACTGCCACTGTGGGTGAGGATGGAAAGGGTCCGCAGATGA
(SEQ ID NO: 922)

Proof-of-principle testing will commence by delivery of a BDC2.5 mRNA sequence, which will be designed as described above. This construct will be compared to the peptide sequence used in the NOD study.

We also designed a cDNA construct that include multiple epitopes/mimotopes for comparison against the single epitope mRNA vaccine in preclinical studies. This construct was designed by placing the mouse transferrin receptor (aa 1-118) domain upstream of the full set of epitopes shown in Table 2. The reverse translated cDNA sequences representing these peptides are shown in Table 48.

TABLE 47
T cell epitopes for tolerance inducing studies in T1D in mice.

| No. | Epitope | Specifications | Sequence | pI | GRAVY |
|---|---|---|---|---|---|
| 1 | InsB | 9-23 | SGLVERLYLVCGERG | 6.47 | 0.013 |
| | | | (SEQ ID NO: 923) | | |
| 2 | GAD65 | 286-300 | KKGAAALGIGTDSVI | 8.59 | 0.560 |
| | | | (SEQ ID NO: 924) | | |
| 3 | InsB | 9-23 R22E | SHLVERLYLVCGEEG | 4.75 | 0.080 |
| | | | (SEQ ID NO: 925) | | |
| 4 | InsB | 9-23 E21G/R22E | SHLVERLYLVCGGEG | 5.38 | 0.287 |
| | | | (SEQ ID NO: 926) | | |
| 5 | mimotope | 2.5mi [p79] | AVPPLWVRME | 6.05 | 0.380 |
| | | | (SEQ ID NO: 927) | | |
| 6 | mimotope | BDC2.5 | RVLPLWVRME | 9.60 | 0.290 |
| | | | (SEQ ID NO: 928) | | |
| 7 | ChgA | 358-371 | WSRMDQLAKELTAE | 4.68 | −0.836 |
| | | | (SEQ ID NO: 929) | | |
| 8 | Hip2.5 | InsC + ChgA | LQTLALWSRMD | 5.84 | 0.109 |
| | | | (SEQ ID NO: 930) | | |
| 9 | InsB | 15-23 | LYLVCGERG | 5.99 | 0.467 |
| | | | (SEQ ID NO: 931) | | |
| 10 | IGRP | 206-214 | VYLKTNVFL | 9.70 | −0.389 |
| | | | (SEQ ID NO: 932) | | |

Accordingly, in certain embodiments the epitopes comprising the tolerogenic nanoparticles described herein comprises one or more of the epitopes shown in Table 47, as well as their corresponding nucleic acid sequences in Table 48 . . .

TABLE 48
Reverse translation of Diabetes T cell epitopes for mRNA preparation.

| No | Epitope | Location | Reverse translated cDNA sequence |
|---|---|---|---|
| 1 | InsB | 9-23 | agccatctggtggaacgcctgtatctggtgtgcggcgaacgcggc |
| | | | (SEQ ID NO: 933) |
| 2 | GAD65 | 286-300 | aaaaaaggcgcggcggcgctgggcattggcaccgatagcgtgatt |
| | | | (SEQ ID NO: 934) |
| 3 | InsB | 9-23 R22E | agccatctggtggaacgcctgtatctggtgtgcggcgaagaaggc |
| | | | (SEQ ID NO: 935) |
| 4 | InsB | 9-23 | agccatctggtggaacgcctgtatctggtgtgcggcggcgaaggc |
| | | E21G/R22E | (SEQ ID NO: 936) |
| 5 | mimotope | 2.5mi[p79] | gcggtgccgccgctgtgggtgcgcatggaa |
| | | | (SEQ ID NO: 937) |
| 6 | Mimotope | BDC2.5 | cgcgtgctgccgctgtgggtgcgcatggaa |
| | | | (SEQ ID NO: 938) |
| 7 | ChgA | 358-371 | tggagccgcatggatcagctggcgaaagaactgaccgcggaa |
| | | | (SEQ ID NO: 939) |
| 8 | Hip2.5 | InsC + ChgA | ctgcagaccctggcgctgtggagccgcatggat |
| | | | (SEQ ID NO: 940) |
| 9 | InsB | 15-23 | ctgtatctggtgtgcggcgaacgcggc |
| | | | (SEQ ID NO: 941) |
| 10 | IGRP | 206-214 | gtgtatctgaaaaccaacgtgtttctg |
| | | | (SEQ ID NO: 942) |

Table 49 shows an optimized codon sequence for multi-epitope expression of the nucleotide sequences listed in Table 48

TABLE 49
Optimized codon sequence for multi-epitope
expression of the nucleotide sequences listed
in Table 48. The complete coding sequence is
comprised of the mouse TfR1-118 domain (cyan),
spliced in upstream of multiple epitopes and
mimotopes that are connected with cleavable
linkers (yellow and green). After the addition
of start (purple) and stop codons (red),
sequence optimization was obtained as described
before. Average GC content: 59.70%.

ATGATGATGGACCAGGCCAGATCAGCCTTCTCCAATCTCTTTGGCGGAG
AGCCTCTTAGCTACACCAGATTTTCGTTGGCGCGGCAAGTGGATGGAGA
CAACTCCCATGTCGAGATGAAACTGGCCGCTGATGAGGAGGAAAATGCA
GATAACAACATGAAGGCCTCTGTGCGCAAACCCAAGCGCTTCAACGGCC
GGCTCTGCTTTGCTGCCATTGCCCTTGTCATCTTCTTCCTCATCGGCTTC
ATGTCTGGCTACCTAGGGTACTGCAAAAGAGTTGAGCAGAAAGAAGAGT
GCGTGAAGCTGGCAGAAACTGAAGAAACAGACAAGTCAGAGACCATGG
AGACAGAAGATGTTGGAGGCGGCTCTCGAGTACGGCGTGGAGGAGGTG
GCTCTCAGAGTCATTTAGTCGAAAGGCTGTACTTGGTGTGTGGAGAACG
GGGGGGCGGAGGTTCTCGGGTGAGAAGGGGAGGCGGGGGCAGCAAGA
AGAAAGGAGCAGCTGCTCTGGGCATTGGAACGGACAGTGTGATAGGCG
GGGGAAGTCGAGTCCGGAGAGGAGGTGGGGGCAGTGACAGCCACCTTG
TAGAGAGGCTCTATCTTGTGTGCGGCGAGGAGGGCGGAGGAGGCTCGC
GTGTTCGAAGGGGAGGCGGTGGCAGCCAGTCCCACCTGGTGGAACGCC
TGTATTTAGTCTGTGGCGGGGAAGGCGGTGGAGGCTCACGGGTCCGCC
GTGGAGGAGGGGGATCCCAAGCTGTGCCACCACTCTGGGTCAGAATGG
AAGGTGGGGGGTCCAGAGTCAGGCGAGGTGGGGGCGGCAGCCAGAGG
GTGCTGCCCCTGTGGGTGAGGATGGAGGGGGGTGGGTCAAGAGTTCGA
CGTGGGGGAGGTGGCTCTCAGTGGTCTCGCATGGATCAATTGGCGAAG
GAGCTGACTGCAGAGGGAGGAGGCAGCAGGGTAAGGCGCGGGGGCGG
TGGGTCACAGCTGCAGACACTGGCCCTATGGAGCCGGATGGACCTCTAC
CTAGTATGTGGGGAGAGAGGTGGGGGGGGGTCCCGGGTTAGGAGGGGT
GGGGGAGGTTCCCCTGTGTATTTGAAGACCAATGTGTTTCTGAGAGTGC
GCCGATGA (SEQ ID NO: 943)

### Design of Tolerogenic Nanoparticles for Treatment of TID in Humans, Premised on the Delivery of One or More T Cell Epitope Sequences

A formidable number of studies have applied an array of methods and approaches to identify T-cell epitopes derived from diabetes-associated antigens (e.g., James et al. (2020) Diabetes, 69:1311-1335; Smith & Peakman (2018) Front. Immunol. 9:392). We relied on these studies to assemble a catalog of CD4 T-cell epitopes that can be considered for tolerogenic immunotherapy in humans. To harvest T-cell epitopes of potential relevance to TID from the IEDB, we first considered selection making at the protein antigen level, as suggested by James et al. (2020) Diabetes 69:1311-1335). The proteins returned from the IEDB search were then individually interrogated to compile an epitope list for further refinement according to the results of T-cell assays and MHC class II binding affinity. The literature database was also reconciled with the IEDB for consistency. This was accomplished by the assignment of IEDB identification numbers by James et al., who also arranged the list of 143 epitopes by antigen source. This list was further refined by using IEDB prediction tools (http://tools.iedb.org/mhcii/) for selecting the most likely 9-mer core sequences, as shown in Table 3. We also used this information to assign peptide p1 and GRAVY values for prediction making about PLGA encapsulation efficacy.

TABLE 50
HLA class II-specific Core epitopes (adapted from James et al. (2020) Diabetes,
69:1311-1335). The sequences in brackets are flanking regions
that are not part of the core 9-mer.

| | Antigen | | | | |
|---|---|---|---|---|---|
| HLA | (Gene) | Position | Sequence | pI | GRAVY |
| DQ2 | INS (Ins) | 78-86 | [QPLALEGSL] | 4.00 | 0.378 |
| | | | (SEQ ID NO: 944) | | |
| | | 80-86 | [LALEGSL] | 4.00 | 1.214 |
| | | | (SEQ ID NO: 945) | | |
| DQ2 | INS (Ins) | 79-87 | [SLQ]PLALEGSLQ | 4.00 | 0.242 |
| trans | | | (SEQ ID NO: 946) | | |
| | | 80-86 | []LALEGSL[] | 4.00 | 1.214 |
| | | | (SEQ ID NO: 947) | | |
| DQ5 | GAD65 | 500-508 | [YEMVFDGKP]QHTNVCFW | 5.32 | −0.300 |
| | (GAD2) | | Y[IP] (SEQ ID NO: 948) | | |
| DQ6 | INS (INS) | 7-15 | [MALWMR]LLPLLALLA[LW | 5.59 | 1.250 |
| | | | GPDPAAA] (SEQ ID NO: 949) | | |
| | | 22-30 | [LALWGPDP]AAAFVNQHL | 5.08 | 0.435 |
| | | | [CGS] (SEQ ID NO: 950) | | |
| | | 37-45 | [HLV]EALYLVCGE[RGFFYT | 6.75 | 0.270 |
| | | | PK] (SEQ ID NO: 951) | | |
| | | 50-58 | [GERGFF]YTPKTRREA[EDL | 6.26 | −1.230 |
| | | | QV] (SEQ ID NO: 952) | | |
| | | 73-81 | [GQVELGGGP]GAGSLQPLA | 3.79 | 0.130 |
| | | | [LE] | | |
| | | | (SEQ ID NO: 953) | | |
| | | 81-89 | [AGSLQPL]ALEGSLQKR[GI | 6.19 | 0.035 |
| | | | VE] | | |
| | | | (SEQ ID NO: 954) | | |
| | | 97-105 | [QCC]TSICSLYQL[ENYCN] | 4.00 | −0.018 |
| | | | (SEQ ID NO: 955) | | |
| DQ8 | CHGA | 347-355 | [WSKMD]QLAKELTAE[] | 4.68 | −0.793 |
| | (CHGA) | | (SEQ ID NO: 956) | | |
| | GAD65 | 126-134 | [YVVKS]FDRSTKVID[FHYP | 6.75 | −0.665 |
| | (GAD2) | | NE] (SEQ ID NO: 957) | | |
| | | 207-215 | [NTNMFT]YEIAPVFVL[LEY | 3.79 | 0.685 |
| | | | VT] (SEQ ID NO: 958) | | |
| | | 236-244 | [PGGSG]DGIFSPGGA[ISNM | 3.80 | 0.050 |
| | | | YA] (SEQ ID NO: 959) | | |
| | | 253-261 | [AMM]IARFKMFPE[VKEKG] | 9.70 | −0.094 |
| | | | (SEQ ID NO: 960) | | |
| | | 264-272 | [EVK]EKGMAALPR[LIAFTS | 6.86 | −0.155 |
| | | | EH] (SEQ ID NO: 961) | | |
| | | 477-485 | [VDKCLE]LAEYLYNII[KNR | 4.87 | −0.370 |
| | | | EG] (SEQ ID NO: 962) | | |
| | | 510-518 | [CFWYI]PPSLRTLED[N] | 4.37 | −0.300 |
| | | | (SEQ ID NO: 963) | | |
| | | 525-533 | [ERMS]RLSKVAPVI[KA] | 11.00 | −0.087 |
| | | | (SEQ ID NO: 964) | | |
| | | 540-548 | [RMME]YGTTMVSYQ[PL] | 6.00 | −0.307 |
| | | | (SEQ ID NO: 965) | | |
| | IA-2 | 202-210 | [SLSY]EPALL(Q->E)PYL[FH | 5.22 | 0.016 |
| | (PTPRN) | | (Q->E)FGS] (SEQ ID NO: 966) | | |
| | | 470-478 | [AAE]EYGYIVTD(Q->E)[KP | 4.14 | −0.494 |
| | | | LS] (SEQ ID NO: 967) | | |
| | | 525-533 | [QNILSLADVT(Q->E)(Q->E) | 3.80 | −0.014 |
| | | | [AGL] (SEQ ID NO: 968) | | |
| | | 551-559 | [TGL(Q->E)IL](Q->E)TGVG | 4.53 | −0.161 |
| | | | (Q->E) (SEQ ID NO: 969) | | |
| | | | REE[AAA] (SEQ ID NO: 970) | | |
| | | 610-618 | [RQHARQQDK]ERLAALGPE | 8.75 | −1.700 |
| | | | (SEQ ID NO: 971) | | |
| | | 622-630 | [GPEGAH]GDTTFEYQD[LC | 4.31 | −1.100 |
| | | | R] (SEQ ID NO: 972) | | |
| | | 653-661 | [EGPPEPS]RVSSVSSQF[SD] | 4.14 | −0.961 |
| | | | (SEQ ID NO: 973) | | |
| | | 664-672 | [FSD]AAQASPSSH[SSTPSW] | 5.08 | −0.689 |
| | | | (SEQ ID NO: 974) | | |
| | | 724-732 | [AEP]NTCATAQGE[GNIKKN] | 6.18 | −1.122 |
| | | | (SEQ ID NO: 975) | | |
| | | 774-782 | [NASPIIEH]DPRMPAYIA[T] | 5.32 | −0.278 |
| | | | (SEQ ID NO: 976) | | |
| | | 834-842 | [DEGASLYH]VYEVNLVSE | 4.39 | −0.361 |
| | | | [H] (SEQ ID NO: 977) | | |
| | | 935-943 | [KGVK]EIDIAATLE[HVRDQ] | 5.48 | −0.517 |
| | | | (SEQ ID NO: 978) | | |
| | | 965-973 | [FALT]AVAEEVNAI[LKALP | 4.53 | 0.837 |
| | | | Q] (SEQ ID NO: 979) | | |
| | IAPP | 74-82 | [VGSNTYGK(R->Cit)]NAVE | 9.70 | −0.700 |
| | (IAPP) | | V LK(R->Cit)E[PL] (SEQ ID | | |
| | | | NO: 980) | | |
| | INS (INS) | 3-11 | [MA]LWMRLLPLL[ALLALW | 5.59 | 1.250 |
| | | | GPDPAAA] (SEQ ID NO: 981) | | |
| | | 12-20 | [LPLL]ALLALWGPD[PAA] | 3.80 | 1.275 |
| | | | (SEQ ID NO: 982) | | |
| | | 21-29 | [LALWGPD]PAAAFVNQH[L | 5.97 | 0.423 |
| | | | CGSHL] (SEQ ID NO: 983) | | |
| | | 29-37 | [FVNQ]HLCGSHLVE[ALY] | 5.98 | 0.469 |
| | | | (SEQ ID NO: 984) | | |
| | | 37-45 | [LCGSHLV]EALYLVCGE[R | 6.74 | 0.438 |
| | | | GFFYTPK] (SEQ ID NO: 985) | | |
| | | 40-48 | YLVCGERGF[FYTPKTR] | 9.19 | −0.419 |
| | | | (SEQ ID NO: 986) | | |
| | | 53-61 | [GERGFFYTP]KTRREAEDL | 6.26 | −1.230 |
| | | | [QV] (SEQ ID NO: 987) | | |
| | | 57-65 | [RR]EAEDLQVGQ[VELGG] | 4.41 | −0.838 |
| | | | (SEQ ID NO: 988) | | |
| | | 59-67 | [A]EDLQVGQVE(SEQ ID | 3.57 | −0.390 |
| | | | NO: 989) | | |
| | | 65-73 | [LQVG]QVELGGGPG[AGS] | 4.00 | 0.156 |
| | | | (SEQ ID NO: 990) | | |
| | | 66-74 | []VELGGGPGA[](SEQ ID | 4.00 | 0.344 |
| | | | NO: 991) | | |
| | | 74-82 | [GGPG]AGSLQPLAL[EGSLQ | 6.14 | −0.087 |
| | | | KRGIVE] (SEQ ID NO: 992) | | |
| | | 85-93 | [AGSLQPLALEG]SLQKRGIV | 6.17 | 0.062 |
| | | | E[QCCT] (SEQ ID NO: 993) | | |
| | | 97-105 | [VEQCC]TSICSLYQL[ENYC | 3.79 | 0.021 |
| | | | N] (SEQ ID NO: 994) | | |
| | INS hybrid | C- | [G]QVELGGGSS[PETLI] | 3.79 | 0.020 |
| | | pep:NP-Y | (SEQ ID NO: 995) | | |
| DQ8 | IA-2 | 146-154 | [LQDI]PTGSAPAAQ[HRLPQ] | 6.74 | −0.606 |
| trans | (PTPRN) | | (SEQ ID NO: 996) | | |
| | INS (INS) | 17-24 | []WGPDPAAA[](SEQ ID | 3.80 | −0.325 |
| | | | NO: 997) | | |
| | | 37-45 | [SHLV]EALYLVCGE[RG] | 5.38 | 0.433 |
| | | | (SEQ ID NO: 998) | | |
| | | 74-82 | [PG]AGSLQPLAL[EGSLQKR] | 9.18 | −0.339 |
| | | | (SEQ ID NO: 999) | | |
| | | 77-85 | [S]LQPLALEGS[L] (SEQ ID | 4.00 | 0.582 |
| | | | NO: 1000) | | |
| DR1 | GAD65 | 89-97 | [N]YAFLHATDL[LP] (SEQ | 5.08 | 0.333 |
| | (GAD2) | | ID NO: 1001) | | |
| | | 249-257 | [M]YAMMIARFK[MF] (SEQ | 9.99 | 0.967 |
| | | | ID NO: 1002) | | |
| | | 418-426 | [GLMQN]CNQMHASYL[FQ | 6.73 | −0.821 |
| | | | QDK] (SEQ ID NO: 1003) | | |
| | | 506-514 | [HTNVC]FWYIPPSLR[TLED | 4.65 | −0.560 |
| | | | NE] (SEQ ID NO: 1004) | | |
| DR3 | GAD65 | 252-260 | [NMYAM]MIARFKMFP[EVK | 9.53 | −0.225 |
| | (GAD2) | | EKG] (SEQ ID NO: 1005) | | |
| | | 340-348 | [TAGTT]VYGAFDPLL[AVA | 3.56 | 0.733 |
| | | | D] (SEQ ID NO: 1006) | | |
| | | 493-501 | [YE]MVFDGKPQH[TNVCFW | 5.32 | −0.300 |
| | | | YIP] (SEQ ID NO: 1007) | | |
| | | 515-523 | [PSLR]TLEDNEERM[SRLSK | 6.68 | −1.015 |
| | | | VA] (SEQ ID NO: 1008) | | |
| | IA-2 | 329-337 | [DRGEKPASPA]VQPDAALQ | 6.12 | −0.242 |
| | (PTPRN) | | R[LAAVL] (SEQ ID NO: 1009) | | |
| | | 474-482 | [SPLGQSQPTVAGQPSARPA | 5.90 | −0.358 |
| | | | AEEYG | | |
| | | | Y]IVTDQKPLS[LAAGVK] | | |
| | | | (SEQ ID NO: 1010) | | |
| | IGRP | 15-23 | [QH]LQKDYRAYY[TF] (SEQ | 8.44 | −1.408 |
| | (G6PC2) | | ID NO: 1011) | | |
| | | 228-236 | [RV]LNIDLLWSV[PI] (SEQ | 5.84 | 5.84 |
| | | | ID NO: 1012) | | |
| | INS (INS) | 48-56 | FFYTPKTRR[EAED] (SEQ ID | 6.18 | −1.562 |
| | | | NO: 1013) | | |
| | ZnT8 | 121-129 | [S]LWLSSKPPS[KRLTFGWH | 11.00 | 0.019 |
| | (SLC30A8) | | RAEILGALL] (SEQ ID | | |
| | | | NO: 1014) | | |
| | | 160-168 | [FGWHRAEILGALLSILCIW | 5.44 | 0.944 |
| | | | VVTGV | | |
| | | | LV]YLACERLLY[PDYQIQ] | | |
| | | | (SEQ ID NO: 1015) | | |
| | | 329-337 | [ASRDSQ]VVRREIAKA[LSK | 8.64 | −0.356 |
| | | | SFTMH | | |
| | | | SLTIQMESPVD] (SEQ ID | | |
| | | | NO: 1016) | | |
| DR0401 | DMK | 19-27 | [RLQQLVLDPG]FLGLEPLLD | 4.03 | 0.570 |
| | (DMPK) | | [L] (SEQ ID NO: 1017) | | |
| | GAD65 | 8-16 | [MASPGSG]FWSFGSEDG[SG | 3.49 | −0.525 |
| | (GAD2) | | DS] (SEQ ID NO: 1018) | | |
| | | 80-88 | [CACDQKP]CSCSKVDVN[Y | 5.93 | 0.115 |
| | | | AFL] (SEQ ID NO: 1019) | | |
| | | 91-99 | [YA]FLHATDLLP[ACDGE(R-> | 4.54 | 0.070 |
| | | | Cit)PTL] (SEQ ID NO: 1020) | | |
| | | 110-108 | [RPTLA]FLQDVMNIL[LQYV | 8.59 | 0.640 |
| | | | VK] (SEQ ID NO: 1021) | | |
| | | 117-125 | [DVMN]ILLQYVVKS[FDRST | 8.50 | 0.185 |
| | | | KV] (SEQ ID NO: 1022) | | |
| | | 117-125 | [MN]ILL(Q->E)YVVKS[FD] | 5.59 | 0.669 |
| | | | (SEQ ID NO: 1023) | | |
| | | 160-168 | [P(Q->E)NLEEI]LMHC(Q->E) | 7.12 | −0.310 |
| | | | TTLK[YAIK] (SEQ ID | | |
| | | | NO: 1024) | | |
| | | 176-184 | [GH]PRYFNQLST[G] (SEQ | 8.75 | −1.108 |
| | | | ID NO: 1025) | | |
| | | 205-213 | [NTNM]FTYEIAPVF[VLLEY | 3.79 | 0.685 |
| | | | VT] (SEQ ID NO: 1026) | | |
| | | 273-281 | [KGMAALPR]LIAFTSEHS[H | 10.29 | −0.104 |
| | | | FSLKK] (SEQ ID NO: 1027) | | |
| | | 273-281 | [KGMAALP(R->Cit)]LIAFTS | 8.76 | 0.080 |
| | | | EHS[HFS] (SEQ ID NO: 1028) | | |
| | | 276-284 | [LIA]FTSEHSHFS[LKKGAA | 8.61 | 0.375 |
| | | | AL] (SEQ ID NO: 1029) | | |
| | | 300-308 | [DSV]ILIKCDERG[KMIPSDL | 4.44 | −0.185 |
| | | | E] (SEQ ID NO: 1030) | | |
| | | 310-318 | [DERGK]MIPSDLERR[ILEA | 6.28 | −1.240 |
| | | | KQI (SEQ ID NO: 1031) | | |
| | | 327-335 | [EAKQKG]FVPFLVSAT[AGT | 8.69 | 0.365 |
| | | | TV] (SEQ ID NO: 1032) | | |
| | | 360-368 | [KYKI]WMHVDAAWG[GG] | 8.51 | −0.307 |
| | | | (SEQ ID NO: 1033) | | |
| | | 379-387 | [KHK]WKLSGVERA[NSVT | 10.46 | −1.386 |
| | | | WNPHK] (SEQ ID NO: 1034) | | |
| | | 435-443 | [YD]LSYDTGDKA[LQCGRH | 4.56 | −0.685 |
| | | | VDV] (SEQ ID NO: 1035) | | |
| | | 482-490 | [KCLELAEYL]YNIIKNREG[ | 4.95 | −0.174 |
| | | | YEMVF] (SEQ ID NO: 1036) | | |
| | | 513-521 | [PS]LRTLEDNEE[RMSR] | 4.87 | −1.693 |
| | | | (SEQ ID NO: 1037) | | |
| | | 556-564 | [VSYQPLGDKVN]FFRMVIS | 8.56 | −0.025 |
| | | | NP(SEQ ID NO: 1038) | | |
| | | 549-557 | [SYQ]PLGDKVNFF[RMV] | 8.31 | −0.220 |
| | | | SEQ ID NO: 1234 | | |
| | | 557-565 | [GDKVNF]FRMVISNPA[ATH | 5.30 | 0.140 |
| | | | QDIDFLI] (SEQ ID NO: 1039) | | |
| | | 557-565 | [KVNF]F(R->Cit)MVISNPA | 6.75 | 0.322 |
| | | | [ATHQDIDFLI] (SEQ ID | | |
| | | | NO: 1040) | | |
| | | 571-579 | [AATHQD]IDFLIEEIE[R] | 4.17 | −0.356 |
| | | | (SEQ ID NO: 1041) | | |
| | GRP78 | 198-206 | [VMR]IINEPTAAA[IAY] | 5.97 | 0.780 |
| | (HSPA5) | | (SEQ ID NO: 1042) | | |
| | | 198-206 | [VM(R->Cit)]IINEPTAAA[IA | 5.97 | 0.780 |
| | | | Y] (SEQ ID NO: 1043) | | |
| | | 501-509 | [EVT]FEIDVNGIL[RVT] | 4.14 | 0.527 |
| | | | (SEQ ID NO: 1044) | | |
| | | 501-509 | [EVT]FEIDVNGIL[(R->Cit) V | 4.14 | 0.527 |
| | | | TAE] (SEQ ID NO: 1045) | | |
| | IA-2 | 661-669 | [VSSQ]FSDAAQASP[SSHSS] | 5.08 | −0.517 |
| | (PTPRN) | | (SEQ ID NO: 1046) | | |
| | | 719-727 | [LAKEWQALCA]YQAEPNT | 4.25 | −0.440 |
| | | | CA | | |
| | | | [TAQGEG] (SEQ ID NO: 1047) | | |
| | | 753-761 | [IK]LKVESSPSR[SDYINASPI] | 8.50 | −0.400 |
| | | | (SEQ ID NO: 1048) | | |
| | | 805-813 | [MVWESGCT]VIVMLTPLV | 3.57 | 1.114 |
| | | | [EDGV] (SEQ ID NO: 1049) | | |
| | | 856-864 | [FY]LKNVQTQET[RTLTQFH | 8.60 | −0.684 |
| | | | F] (SEQ ID NO: 1050) | | |
| | | 959-967 | [SKDQ]FEFALTAVA[EEVNA | 4.41 | 0.143 |
| | | | ILK] (SEQ ID NO: 1051) | | |
| | IGRP | 22-30 | [KDYRA]YYTFLNFMS[NVG | 8.43 | −0.850 |
| | (G6PC2) | | DPR] (SEQ ID NO: 1052) | | |
| | | 25-33 | [YT]FLNFMSNVG[DP] (SEQ | 3.80 | 0.015 |
| | | | ID NO: 1053) | | |
| | | 249-257 | [KWCANPDW]IHIDTTPFA[G | 5.21 | 0.075 |
| | | | LV] (SEQ ID NO: 1054) | | |
| | INS (INS) | 17-25 | [LALLAL]WGPDPAAAF[V] | 3.80 | 1.450 |
| | | | (SEQ ID NO: 1055) | | |
| | | 25-33 | [PAAA]FVNQHLCGS[HLV] | 7.28 | 0.656 |
| | | | (SEQ ID NO: 1056) | | |
| | | 80-88 | [GGGPGAGSLQP]LALEGSL | 6.14 | −0.231 |
| | | | QK[RGIVEQ] (SEQ ID | | |
| | | | NO: 1057) | | |
| | | 77-85 | [S]LQPLALEGS[L] (SEQ ID | 4.00 | 0.582 |
| | | | NO: 1058) | | |
| | | 79-87 | [Q]PLALEGSLQ(SEQ ID | 4.00 | −0.010 |
| | | | NO: 1059) | | |
| | | 80-88 | []LALEGSLQK[](SEQ ID | 6.00 | 0.122 |
| | | | NO: 1060) | | |
| | | 91-99 | [SLQKRG]IVEQCCTSI[CSLY | 7.69 | 0.055 |
| | | | Q] (SEQ ID NO: 1061) | | |
| | S100 (S100B) | 9-17 | [KAM]VALIDVFHQ[YSGRE | 6.75 | −0.370 |
| | | | GDK] (SEQ ID NO: 1062) | | |
| | | 28-36 | [REGDKHK]LKKSELKEL(SE | 9.40 | −1.938 |
| | | | Q ID NO: 1063) | | |
| | | 37-45 | [KHKLKKSELKEL]INNELSH | 8.39 | −1.009 |
| | | | FL[E] (SEQ ID NO: 1064) | | |
| | | 77-85 | [ECDFQEFMA]FVAMVTTAC | 4.13 | 0.172 |
| | | | [HEFFEHE] (SEQ ID | | |
| | | | NO: 1065) | | |
| | ZnT8 | 272-280 | [S]ILLMEGVPK[SLN] (SEQ | 5.72 | 0.577 |
| | (SLC30A8) | | ID NO: 1066) | | |
| DR0404 | GAD65 | 253-261 | [M]IARFKMFPE[VKEKG] | 9.70 | −0.353 |
| | (GAD2) | | (SEQ ID NO: 1067) | | |
| | | 276-284 | []FTSEHSHFS[](SEQ ID | 5.98 | −0.822 |
| | | | NO: 1068) | | |
| | | 557-565 | [NF]FRMVISNPA[AT] (SEQ | 9.75 | 0.400 |
| | | | ID NO: 1069) | | |
| DR12 | GAD65 | 253-261 | [MM]IARFKMFPE[VKEKGM | 9.70 | 0.295 |
| | (GAD2) | | AAL] (SEQ ID NO: 1070) | | |
| DR1601 | GAD65 | 156-164 | [NWELADQPQN]LEEILMHC | 4.00 | −0.790 |
| | (GAD2) | | Q[T] (SEQ ID NO: 1071) | | |
| | INS (INS) | 39-47 | [LVEA]LYLVCGERG[FFYT] | 4.53 | 0.829 |
| | | | (SEQ ID NO: 1072) | | |
| DRB4 | GAD65 | 117-125 | [DVMN]ILLQYVVKS[FDRST | 8.50 | 0.185 |
| | (GAD2) | | KV] (SEQ ID NO: 1073) | | |
| | | 219-227 | [IAPVFVLLEY]VTLKKMREI | 8.50 | 1.055 |
| | | | [I] (SEQ ID NO: 1074) | | |
| | | 273-281 | [LPR]LIAFTSEHS[HFS] (SEQ | 6.92 | 0.027 |
| | | | ID NO: 1075) | | |
| | | 358-367 | [ICKKY]KIWMHVDAA[WG | 9.11 | 0.305 |
| | | | GGLL] (SEQ ID NO: 1076) | | |
| | | 531-539 | [VA]PVIKARMME[YGTTMV | 8.47 | 0.210 |
| | | | SYQ] (SEQ ID NO: 1077) | | |
| | | 556-564 | [KVN]FFRMVISNP[AATHQDID] | 6.75 | −0.185 |
| | | | (SEQ ID NO: 1078) | | |
| | | 565-573 | [NP]AATHQDIDF[LI] (SEQ | 4.20 | −0.023 |
| | | | ID NO: 1079) | | |
| | | 571-579 | [ATHQD]IDFLIEEIE[RLGQDL] | 4.01 | −0.365 |
| | | | (SEQ ID NO: 1080) | | |
| | INS (INS) | 14-22 | [PLLAL]LALWGPDPA[AAF | 3.80 | 0.920 |
| | | | VNQI (SEQ ID NO: 1081) | | |
| DRB5 | GAD65 | 89-97 | [N]YAFLHATDL[LP] (SEQ | 5.08 | 0.333 |
| | (GAD2) | | ID NO: 1082) | | |
| | | 249-257 | [M]YAMMIARFK(SEQ ID | 9.99 | 0.690 |
| | | | NO: 1083) | | |
| DPw2 | GAD65 | 378-386 | [GGGLLMSRKH]KWKLSGV | 11.10 | −0.748 |
| | (GAD2) | | ER[AN] (SEQ ID NO: 1084) | | |
| DP0401 | IA-2 | 832-840 | [L]YHVYEVNLV[SEHIWCE | 4.39 | 0.280 |
| | (PTPRN) | | DFL] (SEQ ID NO: 1085) | | |
| | | 850-858 | [SEHIWCEDF]LVRSFYLKN | 5.42 | −0.215 |
| | | | [VQ] (SEQ ID NO: 1086) | | |

Accordingly, in certain embodiments the epitopes comprising the tolerogenic nanoparticles described herein comprises one or more of the epitopes shown in Table 50 and/or their corresponding nucleic acid sequences.

Utilizing this epitope list, a grading system was developed based on the level of confidence that a given epitope may be functionally related to TID in humans (James et al. (2020) Diabetes, 69:1311-1335). This resulted in 3 out of the original 418 class II-restricted epitopes to receive a scoring grade of A1, namely: GAD65 115-127 (DR0401), GAD65 274-286 (DR0401), and INS 33-47 (B9-23) (DQ8) (Table 4).

TABLE 51
T cell epitope candidates to induce tolerance in Type 1 diabetes.

| No. | Epitope | Specifications | Sequence | pI | GRAVY |
|---|---|---|---|---|---|
| 1 | GAD65 | 115-127 | MNILLQYVVKSFD | 5.59 | 0.669 |
| | | | (SEQ ID NO: 1087) | | |
| 2 | GAD65 | 274-286 | IAFTSEHSHFSLK | 6.92 | −0.092 |
| | | | (SEQ ID NO: 1088) | | |
| 3 | Insulin | 33-47 | SHLVEALYLVCGERG | 5.38 | 0.433 |
| | | | (SEQ ID NO: 1089) | | |

Accordingly, in certain embodiments the epitopes comprising the tolerogenic nanoparticles described herein comprises one or more of the epitopes shown in Table 51 and/or their corresponding nucleic acids.

Of particular interest is the delineation of epitope sequences that bind to HLA-DRB1*0401, which when combined with DQA1*03:01 or DQB1*03:02 are referred to as “DR4 haplotypes” (Noble et al. (2015) J. Autoimmunity, 64: 101e112). DRB1*04:01 has a distinct motif that can interact with and present a proinsulin epitope, associated with TID in humans (Tait et al. (1995) Eur. J. Immunogenet. 22 (4): 289-297). In essence, DRB4*01:01 (DRB4) is a secondary HLA-DR product that is part of the high-risk DR4/DQ8 haplotype, which is associated with TID (James et al. (2018) J. Immunol. doi: 10.4049/jimmunol. 1800723. DRB4). DRB4 shares homology with HLA-DR4, which also predisposes to multiple sclerosis, rheumatoid arthritis, and lyme disease induced arthritis.

Based on the above information, we propose a personalized medicine approach to develop TID immunotherapy for individuals with a DRB1*0401 genotype and its associated DR4 haplotypes. The immunotherapy will be delivered in the form of tolerogenic nanoparticles that can deliver one or more of the peptides or corresponding nucleic acid sequences in Table 51 to the liver. Reverse translation of the peptide sequences yield the nucleotide sequences shown in in Table 52.

TABLE 52
Reverse translated T cell epitopes for mRNA preparation.

| No. | Epitope | Specifications | Sequence |
|---|---|---|---|
| 1 | GAD65 | 115-127 | atgaacattctgctgcagtatgtggtgaaaagctttgat |
| | | | (SEQ ID NO: 1090) |
| 2 | GAD65 | 274-286 | attgcgtttaccagcgaacatagccattttagcctgaaa |
| | | | (SEQ ID NO: 1091) |
| 3 | Ins | 33-47 | agccatctggtggaagcgctgtatctggtgtgcggcgaacgcggc |
| | | | (SEQ ID NO: 1092) |

In the multi-epitope vaccine shown in Table 53 we show the insertion of the human transferrin receptor (aa 1-118) domain upstream of the combined nucleotide sequences, separated by cleavable linkers. Following the addition of start and stop codons, codon optimization was performed for human use (Table 53). This coding sequence will be inserted into a pTNT plasmid, as described above.

TABLE 53
Codon-optimized multi-epitope vaccine
for use in humans with T1D and displaying a
DRB1*0401 genotype.

ATGATGGACCAGGCCAGAAGCGCCTTTAGCAACCTGTTCGGCGGAGAGCC
TCTGAGCTACACCAGATTCAGCCTGGCTAGACAGGTGGATGGCGACAATA
GCCACGTGGAAATGAAACTGGCCGTGGACGAGGAAGAGAACGCCGATAA
CAACACCAAGGCCAATGTGACAAAACCTAAGCGGTGCAGCGGCTCCATCT
GCTACGGCACCATCGCCGTGATCGTGTTCTTCCTGATCGGCTTTATGATC
GGCTATCTGGGCTACTGCAAGGGCGTGGAACCCAAGACCGAGTGTGAAAG
ACTGGCTGGCACAGAGAGCCCTGTGCGGGAAGAACCAGGCGAGGACTTCC
CCGCCGGCGGAGGCGGCGGCTCCAGAGTGCGGAGAGGCGGCGGCGGCTC
TATGAACATCCTGCTGCAGTACGTGGTGAAGAGCTTCGACGGCGGCGGCG
GCTCTAGAGTGCGCAGAGGCGGCGGCGGATCTATTGCCTTCACCAGCGAG
CACAGCCATTTCAGCCTGAAGGGCGGAGGCGGATCTAGGGTTAGACGGG
GAGGAGGAGGTTCCAGCCACCTGGTCGAGGCCCTGTACCTCGTGTGCGGC
GAGAGAGGCAGAGTCCGGCGGTGA (SEQ ID NO: 1093)

Successful tolerogenic mRNA nanoparticle therapy will allow the personalized immunotherapy approach to be further explored in additional TID subsets where information is available about HLA genotype and the autoantigen involved based on autoantibody profiles. Epitope predictions for those antigens, coupled with MHC-II binding analysis will allow the development of additional single or multi-epitope mRNA vaccines.

### Histone Peptide Epitopes for Tolerogenic Therapy in Lupus

It has been demonstrated that autoantigen-specific tolerance can be induced in genetically susceptible lupus-prone mouse strains treated with nucleosomal histone peptide epitope(s) (see, e.g., Datta (2021) Font. Immunol. 12: Article 629807; Zhang et al. (2103) Clin Immunol 149 (3): doi: 10.1016/j.clim.2013.08.008; Hahn et al. (2008) Arth & Rheum. 58:2488-2497; Datta (2003) Ann. N.Y. Acad. Sci. 987:79-90). Low-dose therapy with these epitopes can generate CD4⁺CD25⁺ Tregs and CD8⁺ Tregs, capable of suppressing pathogenic autoimmune responses (e.g., kidney damage). The effect can be adoptively transferred to recipient animals. Similar potent Treg cells can also be generated ex vivo in peripheral blood mononuclear cells (PBMC) from lupus patients during histone epitope exposure, resulting in interference of anti-dsDNA antibody production (Datta (2021) Font. Immunol. 12: Article 629807).

The mechanistic basis for considering the use of histone peptide epitopes is based on nucleosome accumulation in lupus patients due to defective clearance of apoptotic cells. This can happen due to a number of reasons. One important contribution to defective clearance are abnormalities or deficiencies of the complement system (e.g., Clq), which plays a role in non-immunogenic clearance of apoptotic cells. Another contributing factor may be a deficiency of scavenger receptors in phagocytic cells (e.g., Marcos), which leads to the activation of professional APCs such as dendritic cells as well as B cells. This allows B cells to participate in the presentation of the autoantigens that appear in apoptotic microparticles. Lupus pathogenesis is dependent on the presence of intact histones, DNA, RNA, and HMGB-1 in the apoptotic microparticles. Not only can these components act as autoantigens, but are also capable of activating endogenous toll-like receptors (TLR), which contribute to innate and adaptive immune responses in lupus patients. Accordingly, immunization with intact nucleosome particles can induce kidney disease in lupus-prone mice. In contrast, uptake of the microparticles by tangible-body macrophages and plasmocytoid dendritic cells in normal mice, is responsible for inducing immune tolerance in response to apoptotic cells.

The tolerogenic effects of histone peptide epitopes are unique in their ability to suppress cross-reactive immunity, which forms the basis of the epitope spreading effect when APC in lupus patient's indirect with autoimmune B- and T-cells (see, e.g., Datta (2021) Front. Immunol. 12: Article 629807). The cross-reactivity is augmented by autoantibody production to nucleic acid and nucleosome components, also allowing B-cells to to take up the role of APCs, which interact with lupus T-helper (TH) cells that could already be activated by dendritic cells. These cooperative and multiplicative cellular interactions drive “linked recognition” of diverse antigens presented by an array B-cells to a single interactive TH cell. The TH, in turn, enhances antibody production to histones, DNA, RNA and HMGB-1. Thus, a single lupus TH clone is able to assist multiple B-cells in promoting antibody production to nucleosomes, dsDNA, ssDNA, histone or HMGB-1. Contrariwise, the generation of Tregs in response histone peptide epitopes can allow the tolerogenic response mediated by a single T cell epitope to interfere in antibody production by multiple B-cell subsets.

While the initial search for histone peptide epitopes yielded >150 overlapping 15-mer peptides spanning the length of all 4 core histones, the use of specific autoimmune TH cells retrieved from lupus mouse models were able to limit the number of core epitopes (Lu et al. (1999) J. Clin. Invest. 104 (3): 345-355; Datta (2021) Front. Immunol. 12: Article 629807; Hahn et al. (2008) Arth & Rheum. 58:2488-2497; Datta (2003) Ann. N.Y. Acad. Sci. 987:79-90; Kaliyaperumal et al. (1996) J. Exp. Med. 183:2459-69). This led to initial identification of a short list of critical autoepitopes being recognized by lupus TH cells in mice with lupus nephritis: aa 10-33 in histone H-2B, aa 16-39 and 71-94 in H4, aa 85-102 in H3 and aa 22-42 in H1 (Kaliyaperumal et al. (1996); J Exp Med; 1996; 183:2459-69). Further refinement brought about by immunotherapy studies in lupus mice, ultimately led to the identification of the following major autoepitopes recognized by TH cells: H1′ aa 22-42, H3 aa 85-102, H4 aa 16-39, and H4 aa 71-94 (Datta SK; Frontiers in Immunology; 2021: Volume 12: Article 629807). These histone epitopes are capable of stimulating cellular responses in autoimmune TH cells and B cells. The presence of these peptide sequences in regions where histones make contact with DNA, suggests that these epitopes are protected from degradation during autoantigen processing, leading to antigen presentation by TH cells.

Histone peptides have been shown to be effective in animal studies during delivery of a single or combination of epitopes. When injected in the absence of adjuvants, even one of these epitopes can lead to a tolerogenic impact on autoimmune TH cells of diverse autoantigen specificity. Conversely suppressing a TH cell with specificity for a single nuclear autoantigenic epitope, can lead to the deprivation of the input for multiple B cells engaged in lupus autoantibody production (Datta (2021) supra.). This blocks epitope spreading, with the implication that “cross-reactive suppression” of a broad spectrum of autoimmune responses by a single epitope could be as effective as targeting multiple epitopes individually in a lupus mouse. However, combined use of a mixture of the peptide epitopes could be more effective in humans due to the heterogeneity of the HLA alleles compared to limited diversity in inbred mouse strains (Zhang et al. (2013) Clin. Immunol. 149 (3): doi: 10.1016/j.clim.2013.08.008). Thus, a cocktail of peptides has proven to be more effective in suppressing autoimmunity in human lupus PBMC cultures, compared to a single epitope. It is possible, therefore, that a mixture of histone peptide epitopes could lead to more durable suppression of autoimmune responses in human lupus by impacting diverse pathways.

Low-dose tolerance therapy was developed in lupus-prone mice by injecting nucleosomal histone peptide autoepitopes subcutaneously every 2 weeks (Kang et al. (2005) J. Immunol. 174:3247-3255). Subnanomolar dosing had the effect of lowering autoantibody levels, preventing inflammatory infiltrates in the kidney, reducing lupus nephritis, and leading to restoration of a normal life span. H4 aa 71-94 was the most effective autoepitope in in these studies. The therapeutic response was accompanied by the generation of regulatory CD8⁺ cell subsets as well as autoantigen-specific and cross-reactive CD4⁺CD25⁺ Tregs. The induced CD4⁺CD25⁺ and CD8⁺ Tregs cells utilize TGF-beta production to induce immunosuppression (Kang H-K, et al.). The Tregs were also effective in suppressing lupus autoimmunity upon adoptive transfer. While the effect of CD4⁺CD25⁺ Tregs was partially dependent on cell-cell contact, CD8⁺ Tregs could function contact-independently.

Against this background, the use of histone peptide tolerance provides a natural and safe approach for lupus treatment in humans. Noteworthy, the TH present in the PBMC retrieved from human lupus patients have been shown to recognize the same immunodominant histone peptide sequences that play a role in anti-DNA autoantibody production, and lupus nephritis in mice ((Datta (2021) supra.; Kaliyaperumal et al. (1996) J. Exp. Med. 183:2459-2469); Lu et al. (1999) J. Clin. Invest. 104:345-355). Moreover, these peptides promiscuously interact with HLA-DR, and represent nucleosome sequences making contact with DNA, as well as inducing autoantibody production, similar to lupus mice. Another similarity is that the addition of the histone peptide epitopes are capable of inducing CD4⁺CD25high FoxP3⁺ and CD4⁺CD45RA⁺FoxP3 Tregs as well as CD8⁺CD25⁺FoxP3⁺ Tregs in human PBMC. Noteworthy, the appearance of Tregs in human blood was observed even for patients on maintenance therapy with anti-inflammatory drugs such as hydroxyl-chloroquine or corticosteroids (Giles et al. (2017) JCI Insight. 2 (4): e90870. doi: 10.1172/jci.insight.90870). We have already referred to the finding that a mixture of peptide epitopes was more effective in suppressing pathogenic activity in human PBMC cultures than a single epitope.

We have undertaken our own study to look at the effect of a tolerogenic epitope for the treatment of a lupus animal model in collaboration with Dr. Antonio La Cava's laboratory at UCLA. The tolerogenic nanoparticles were synthesized and characterized in Dr. Nel's laboratory, while the animal work was carried out in Dr. La Cava's laboratory. The original discovery of tolerogenic epitopes in the MRL/lpr and (NZB/NZW) FI murine models was made in the laboratory of Dr. Sylviane Muller, who identified the aa 131-151 epitope sequence of the U1-70K spliceosome protein as a CD4 cell epitope (Monneaux et al. (2004), 50 (10): 3232-3238). Peripheral blood lymphocytes (PBLs) from unprimed MRL/Ipr mice, as well as immunized BALB/c mice were tested for their proliferative response to 18 overlapping peptides from the U1-70K protein. This demonstrated a rapid evolution of the proliferative response to different epitope sequences in different age groups. At least 2 major peptides recognized by MRL/lpr PBLs were also recognized by PBLs generated in the BALB/c mice that were primed with the aa 131-151 peptide. It was further demonstrated in pre-autoimmune MRL/Ipr mice that repeated administration of phosphorylated version of the 131-151 peptide (called P140), is capable of transiently abolishing T-cell epitope spreading to other U1-70K regions (Monneaux et al. supra.). Subsequently, the U1-70K snRNP self-antigen and corresponding epitopes have been shown to be prime targets for autoimmune responses mediated by antigen-specific CD4 T-cells. Moreover, it has been demonstrated that administration of these peptides is capable of reversing autoimmune manifestations and promoting animal survival by inhibiting autoreactive CD4 T-cells (Schall & Muller (2015) Lupus, 24, 412-418). P140 (a.k.a. Lupuzor) has subsequently been shown to be effective in treating lupus patients, with evidence of therapeutic safety and efficacy in Phase 1 and Phase 2 clinical trials (Zimmer et al. (2013) Ann Rheum Dis. 72 (11): 1830-1835; ClinicalTrials.gov Identifier: NCT01135459).

To test disease modulation in MRL/Ipr mice through the use of tolerogenic nanoparticles, we encapsulated the snRNP-derived P140 peptide (RIHMVYSKRpSGKPRGYAFIEY (SEQ ID NO:1094)) into PLGA NPs. The carrier was synthesized as previously described for allergenic protein peptides. Utilizing particles with a peptide content of 37.88 ug/ml, individual MRL/lpr mice received an IV injection of 500 ug NPs, starting at 4 weeks of age. Our principal hypothesis was that targeted delivery of ApoBP-decorated PLGA nanoparticles will allow P140 presentation by liver sinusoidal endothelial cells, capable of generating disease-suppressing Tregs. The proposed therapeutic advantage for nanocarrier use was to exploit the natural tolerogenic effects of LSECs, rather than relying on free peptide access to any number of non-characterized APC. Six mice were used in each group. In addition to animals receiving NPs encapsulating P140, other groups included mice treated with NPs encapsulating an irrelevant peptide (irr peptide), mice treated with empty NPs, and mice receiving saline alone (vehicle). The animals were monitored for the development of lupus nephritis by assessing protein content in the urine. Additional studies to demonstrate the involvement of Tregs and abatement of autoimmune phenomena are ongoing,

The results for assessment of proteinuria, as a reflection of lupus nephritis severity, are shown in FIG. 11. The data demonstrate that, compared to the saline control, empty nanoparticles or use of a control peptide, the nanoparticles encapsulating P140 could totally suppress the protein leakage through the kidney. The impact on lifespan, tissue histology, autoantibody production and the generation of Tregs is being determined. Moreover, the animals will also be monitored for development of additional lupus manifestations that include arthritis, cerebritis, skin rash and vasculitis,

These findings provide a proof-of-concept of the validity of TNP delivery of a spliceosome peptide in preventing lupus nephritis in MRL/Ipr mice. We suspect that the outcome is the result of Treg generation by targeted LSECs.

Without being bound to a particular theory, it is believed that histone peptide epitopes would be effective tolerogens for inhibiting autoimmune T and B-cell populations in lupus patients with diverse HLA alleles. Given the overlap of the murine with human epitope sequences, including the promiscuous binding interactions of the peptides to human HLA-DR alleles, we have assembled the histone auto-epitope sequences shown in Table 1 to design tolerogenic nanoparticles for proof-of-principle studies in animals. By implication, these preclinical studies would be directly relevant to the planning of therapeutic intervention in lupus patients due to sequence similarity. The peptide sequences, including for a control peptide, were also used to assess peptide charge (p1) and solubility (GRAVY criteria). According to this prediction making, virtually all the peptides would qualify for PLGA encapsulation, with the exception of the histone H3 82-105 sequence.

TABLE 54
Histone auto-epitopes for therapeutic studies in preclinical animal models as
well as in lupus human lupus subjects, who produce anti-DNA antibodies.

| No. | Epitope | Location | Sequence | pI | GRAVY |
|---|---|---|---|---|---|
| 1 | Histone H1 | 22-42 | STDHPKYSDMIVAAIQAEKNR | 6.47 | −0.843 |
| | | | (SEQ ID NO: 1095) | | |
| 2 | Histone H3 | 82-105 | DLRFQSSAVMALQEACEAYLVGLF | 4.14 | 0.646 |
| | | | (SEQ ID NO: 1096) | | |
| 3 | Histone H3 | 115-135 | KRVTIMPKDIQLARRIRGERA | 11.83 | −0.786 |
| | | | (SEQ ID NO: 1097) | | |
| 4 | Histone H4 | 16-32 | KRHRKVLRDNIQGITKP | 11.73 | −1.447 |
| | | | (SEQ ID NO: 1098) | | |
| 5 | Histone H4 | 71-94 | TYTEHAKRKTVAMDVVYALKRQG | 9.82 | −0.687 |
| | | | (SEQ ID NO: 1099) | | |
| 6 | Control | HDL-BP | EIAEEYGGVMVSFPR | 4.25 | −0.007 |
| | Peptide A | 525-839 | (SEQ ID NO: 1100) | | |

Accordingly, in certain embodiments the epitopes comprising the tolerogenic nanoparticles described herein comprises one or more of the epitopes shown in Table 54 and/or the corresponding nucleic acid sequences.

Single and multiple epitope delivering nanoparticles are being constructed for animal studies in lupus-prone mice [e.g., the SNF1 (SWR×NZB) F1 lupus nephritis model]. The single epitope selection will commence with the H4 71-94 peptide sequence, as the most effective epitope in historical records (Kang et al. (2005) J. Immunol. 174:3247-1355). The effect will be compared to particles delivering a control peptide as well as particles that encapsulate P140. Ultimately, single epitope delivery will be compared to multi-epitope nanoparticles, as well as the same range of nanoparticles delivering mRNA constructs. The design of the mRNA nanoparticles will proceed along the method outlined above. FIGS. 2 and 3 below show the design for mRNA constructs that encode for just the H4 71-94 epitope or the control peptide. The second approach will be to develop mRNA that encode for peptides 1-5 in Table 54. Table 55 shows reverse translation of the sequences.

TABLE 55
Reverse translation of histone auto-epitopes for therapeutic studies.

| No. | Epitope | Location | Reverse translated cDNA sequence |
|---|---|---|---|
| 1 | Histone H1 | 22-42 | agcaccgatcatccgaaatatagcgatatgattgtggcggcgattcaggcggaaaaa |
| | | | aaccgc |
| | | | (SEQ ID NO: 1101) |
| 2 | Histone H3 | 82-105 | gatctgcgctttcagagcagcgcggtgatggcgctgcaggaagcgtgcgaagcgt |
| | | | atctggtgggcctgttt |
| | | | (SEQ ID NO: 1102) |
| 3 | Histone H3 | 115-135 | aaacgcgtgaccattatgccgaaagatattcagctggcgcgccgcattcgcggcga |
| | | | acgcgcg |
| | | | (SEQ ID NO: 1103) |
| 4 | Histone H4 | 16-32 | aaacgccatcgcaaagtgctgcgcgataacattcagggcattaccaaaccg |
| | | | (SEQ ID NO: 1104) |
| 5 | Histone H4 | 71-94 | acctataccgaacatgcgaaacgcaaaaccgtggcgatggatgtggtgtatgcg |
| | | | ctgaaacgccagggc |
| | | | (SEQ ID NO: 1105) |
| 6 | Control | HDL-BP | gaaattgcggaagaatatggcggcgtgatggtgagctttccgcgc |
| | Peptide A | 525-839 | (SEQ ID NO: 1106) |

Additional cDNA design involved placement of the mouse transferrin receptor 1 (1-118) domain sequence upstream of single or multi-epitope sequences, interspaced by cleavable linkers. Following the addition of start and stop codons, codon optimization was used for murine expression (Tables 56, 57, and 58). The corresponding mRNA will be synthesized using a pTNT plasmid for, as described above.

TABLE 56
Codon-optimized Control peptide A coding sequence
for murine pre-clinical studies: The coding
sequence includes the mouse TfR1-118 domain
(cyan) upstream of control peptide cell epitopes,
which are interspaced with cleavable linkers
(yellow and green), plus the addition of start
(purple) and stop codons (red). Average GC
content: 52.72%.

ATGATGATGGACCAGGCACGGAGTGCCTTCAGCAATCTCTTTGGAGGGG
AGCCTCTGTCCTATACTCGCTTCTCTTTAGCACGGCAGGTGGATGGAGA
CAACAGCCATGTTGAGATGAAGCTTGCAGCAGATGAGGAAGAAAATGCT
GATAACAACATGAAAGCCAGTGTACGTAAACCAAAAAGGTTTAATGGCC
GCCTCTGCTTTGCTGCCATAGCGTTGGTCATCTTTTTCCTGATTGGCTTC
ATGTCTGGCTACCTAGGATACTGCAAGAGAGTGGAGCAGAAGGAAGAAT
GTGTGAAGCTGGCTGAGACCGAGGAGACAGACAAATCAGAAACCATGG
AGACTGAAGACGTAGGTGGGGGCTCCAGAGTCAGAAGAGGTGGCGGAG
GGTCACAGACCTACACAGAGCACGCCAAGAGGAAAACGGTTGCCATGG
ATGTGGTGTATGCTCTGAAGCGGCAAGGGCGAGTCAGGCGATGA
(SEQ ID NO: 1107)

TABLE 57
Codon optimized H4 71-94 cDNA sequence
for murine pre-clinical studies. The
coding sequence includes the mouse
TfR1-118 domain (cyan) upstream of
control peptide cell epitopes, which are
interspaced with cleavable linkers
(yellow and green), plus the addition of
start (purple) and stop codons (red).
Average GC content: 51.35%.

ATGATGATGGACCAGGCACGGAGTGCCTTCAGCAATCTCTTTGGAGGGGA
GCCTCTGTCCTATACTCGCTTCTCTTTAGCACGGCAGGTGGATGGAGACA
ACAGCCATGTTGAGATGAAGCTTGCAGCAGATGAGGAAGAAAATGCTGAT
AACAACATGAAAGCCAGTGTACGTAAACCAAAAAGGTTTAATGGCCGCCT
CTGCTTTGCTGCCATAGCGTTGGTCATCTTTTTCCTGATTGGCTTCATGT
CTGGCTACCTAGGATACTGCAAGAGAGTGGAGCAGAAGGAAGAATGTGTG
AAGCTGGCTGAGACCGAGGAGACAGACAAATCAGAAACCATGGAGACTGA
AGACGTAGGTGGGGGCTCCAGAGTCAGAAGAGGTGGCGGAGGGTCACAGA
CCTACACAGAGCACGCCAAGAGGAAAACGGTTGCCATGGATGTGGTGTAT
GCTCTGAAGCGGCAAGGGCGAGTCAGGCGATGA(SEQ ID NO: 1108)

TABLE 58
Codon-optimized sequence for epitopes #1-5
(Table 54) in murine preclinical studies.
The coding sequence includes the mouse
TfR1-118 (cyan) upstream of control
peptide cell epitopes, which are inter-
spaced with cleavable linkers (yellow and
green), plus the addition of start (purple)
and stop codons (red). Average GC content:
56.26%.

ATGATGATGGATCAGGCCCGATCAGCCTTCAGCAACCTCTTTGGAGGCGA
GCCTCTATCTTATACTAGATTTTCTTTAGCTAGGCAAGTGGATGGAGACA
ATTCTCATGTCGAGATGAAACTTGCAGCAGATGAGGAAGAGAATGCAGAC
AACAACATGAAGGCCTCAGTGAGAAAACCAAAAAGGTTCAATGGCCGCCT
CTGCTTTGCTGCAATTGCTCTGGTCATCTTCTTTCTGATAGGCTTCATGA
GTGGCTATCTGGGTTACTGCAAACGAGTAGAGCAGAAGGAAGAATGTGTG
AAACTTGCAGAAACTGAGGAGACTGACAAATCAGAGACCATGGAGACAGA
AGACGTTGGAGGAGGGAGCAGAGTTCGCCGCGGGGGAGGAGGAAGCCAGA
GCACAGACCACCCCAAGTACAGTGACATGATTGTGGCCGCTATCCAAGCA
GAGAAGAATAGAGGCGGCGGCTCCAGGGTCCGCAGGGGCGGCGGTGGGTC
CAAGGACCTGCGTTTCCAGTCCTCTGCTGTGATGGCGCTGCAGGAGGCCT
GTGAAGCGTACTTGGTGGGCTTGTTTGGGGGCGGTTCACGCGTCAGAAGG
GGTGGGGGAGGTTCCGACAAGCGGGTCACCATCATGCCAAAGGATATTCA
GCTGGCCCGGAGGATACGGGGAGAAAGGGCTGGTGGGGGTAGTCGTGTAC
GTAGAGGGGGCGGAGGCTCTGTCAAGCGACACAGGAAGGTGCTCCGGGAT
AACATCCAGGGCATCACCAAGCCTGGGGGTGGAAGCAGAGTACGCAGGGG
GGGGGGAGGAAGCCCCACCTACACGGAACATGCCAAGCGGAAAACAGTTG
CCATGGATGTGGTGTATGCTTTGAAAAGACAAGGGAGAGTGCGGCGCTGA
(SEQ ID NO: 1109)

Nanoparticles that perform successfully in preclinical studies can be used, to determine the impact on human PBMC from lupus patients, including induction of CD4⁺CD25^{high} FoxP3⁺ and CD4⁺CD45RA⁺FoxP3 Tregs, as well as CD8⁺CD25⁺FoxP3⁺ Tregs, as described by Giles et al. (2017) JCI Insight. 2 (4): e90870. doi: 10.1172/jci.insight.90870). In these studies, the codon sequences will be adapted for human use, including using an upstream human TfR1-118 sequence to direct epitope expression to the MHC-II compartment.

### T-Cell Epitopes Appearing in AAV Viral Vectors for Immune Tolerization

Viral vectors have emerged as a preferred platform for gene delivery for a number of inherited and acquired diseases. Once the viral genome is replaced with a therapeutic gene cassette, which also leads to the removal of the viral replicative and pathogenic traits, such vectors are well suited as gene transfer vehicles. An ideal gene therapy vector should reliably and efficiently carry and deliver a therapeutic gene to target cells and direct long-term therapeutic expression. Viruses naturally satisfy these criteria, except that they are prone to be recognized by the host immune system, which has evolved to recognize infectious agents. Past and ongoing clinical trials have utilized several different viral vectors, including adenovirus (Ad), lentivirus (LV), murine g-retrovirus, herpes simplex virus (HSV) and adeno-associated virus (AAV). Marketing approval has been granted to two AAV-based therapies to treat a form of congenital blindness (Luxturna) and spinal muscular atrophy (Zolgensma), g-retrovirus-based therapy for the primary immune deficiency disorder, known as adenosine deaminase severe combined immunodeficiency (ADA-SCID) (Strimvelis), LV-based therapy for CD19-directed CAR-T cells to treat acute lymphoblastic leukemia and non-Hodgkin lymphoma (Kymriah and Yescarta), and HSV-based oncolytic virotherapy for melanoma (Imlygic) (Shirley et al. (2020) Molecular Therapy, 28:709).

Gene therapy mediated by recombinant adeno-associated virus (AAV) vectors is one of the most promising approaches for treating a variety of inherited and acquired diseases. AAV is a small non-enveloped parvovirus with a single-stranded genome of about 5 kb that is naturally non-pathogenic and replication defective ((Meliani et al. (2018) Nature Communications, 9:4098; Shirley et al. (2929) Molecular Therapy 28:709-722; Hui et al.; (2015) Molecular Therapy, 2:15029; doi: 10.1038/mtm.2015.29; Chen et al. (2006) Molecular Therapy, 13 (2): 260-269). AAV vectors do not contain viral coding sequences and elicit weak inflammatory or type 1 IFN responses only. Moreover, these viral genomes persist mostly in episomal form. AAV vectors are widely used in human gene therapy trials to target a large number of different tissues and cells types, including the CNS, liver, skeletal and cardiac muscle, eye, and lung (Shirley et al. (2020) Molecular Therapy, 28, 709). In addition to two US Food and Drug Administration (FDA)-approved products for treatment of Leber's congenital amaurosis (LCA) and spinal muscular atrophy (SMA), other products have reached phase III trials as in liver-directed gene therapy for hemophilia A and B (Id.). Human clinical gene therapy trials with AAV have demonstrated durable expression at therapeutic levels when targeting tissues like the liver, motor neurons, and the retina. Despite the exciting results to date, one of the limitations of AAV gene therapy is efficacy interference upon multiple administrations (Meliani et al. supra.). For many metabolic and degenerative diseases, treatment is critically needed early in life, prior to the onset of irreversible tissue damage. However, because of their non-integrative nature, systemic gene therapy with AAV vectors in pediatric patients is expected to be limited by tissue proliferation associated with organ growth, which could result in significant vector dilution over time. Thus, maintaining the possibility to re-administer AAV is an important goal to achieve sustained therapeutic efficacy over time in pediatric patients. In addition, vector re-administration in both pediatric and adult patients would be desirable to enable vector titration, to increase the proportion of patients that achieve therapeutic levels of the transgene expression, while avoiding supra-physiological transgene expression and potential toxicities associated with large vector doses (Meliani et al. supra.).

Although AAV vectors are associated with low immunogenicity and toxicity, resulting in vector persistence and long-term transgene expression, vector immunogenicity represents a major limitation to re-administration of AAV vectors. Although AAV vectors do not contain any viral open reading frames, the transgene product and the virus capsid can serve as the sources of antigenicity. Recent studies have shown that AAV vectors can elicit both a cellular and a humoral immune response against the vector and transgene product. The specificity of the CD8 T cell responses to a virus or transgene is determined by virus capsid or transgene-derived peptides of approximately 9 amino acids (AA) in length. These peptides are presented in the context of different class I molecules encoded by the major histocompatibility complex (MHC), which in the mouse is composed of K, D, and L loci. Th2 responses, characterized by production of IL-4 and IL-10, are necessary for immunoglobulin class switching to high-affinity IgG and affinity maturation of the antibody response. Both the Th1 and the Th2 responses require antigen processing and MHC-II presentation of peptides of approximately 15 amino acids in length. For class II MHC antigen presentation, several peptides derived from viral capsid components or from the transgene products, can be presented in the context of either mouse I-A or mouse I-E class II MHC to simulate Th1 or Th2 T cell responses. This class II MHC/peptide complex is specifically recognized by a T cell receptor on CD4⁺ Th1 or Th2 T cells. Identification of specific virus capsid or transgene peptides that interact with class I or class II MHC is important to enable specific analysis of the Th1, Th2, or CTL response after AAV administration.

To induce immune tolerance to AAV, it is important to consider not only the prevention of antibody responses to allow for vector administration, but also the CD8⁺ T cell responses. Results in human trials indicate that induction of CD8⁺ T cell responses directed to the AAV capsid correlates with liver inflammation and can limit the duration of transgene expression. While corticosteroid administration has been effective in blocking these responses, in other instances expression was lost despite immunosuppression. This indicates the need to develop more effective strategies (Meliani et al. supra.). As discussed in the preceding sections, LSEC-mediated antigen presentation to naive CD4+ T cells results in differentiation of CD4+ T cells into antigen-specific TGF-β-producing Foxp3⁺ Tregs, capable of inducing immune tolerance. In addition, LSEC-mediated presentation of antigens by MHC-I to CD8+ T cells can result in tolerogenic rather than effector responses. Direct recognition of antigen presented on LSECs to naive CD8+ T cells can also result in cell activation and induction of apoptosis. The apoptotic cells can be phagocytosed by Kupffer cells, which can further contribute to tolerance induction, in addition to the APC role of LSECs (FIG. 3) (Crispe et al. (2003) Nature Reviews Immunology, 3:51).

Our approach to developing tolerogenic immunotherapy was to collect AAV T-cell epitopes from the literature. The epitope selection was further refined to collect core peptide sequences as described for other immunotherapy applications in this disclosure. The approach that we outline here, can also be applied to other viral gene vectors. Chen et al. (2006) Gene Therapy, Molecular Therapy, 13:260-269 described the application of AAV2 or AAV8 vectors for delivery of human coagulation factor IX (hF.IX) as a promising gene therapy for hemophilia B. One major limitation of this therapy is the development of antibodies and a cytotoxic T lymphocyte (CTL) response against both the vector capsid and the transgene. The authors determined the class I and class II MHC peptide epitopes for AAV2, AAV8, and hF.IX after administration of AAV-2-hF.IX or AAV8-hF.IX in H2b (C57BL/6), H2d (BALB/c), or H2k (C3H) murine strains.

The authors made predictions for class I MHC-restricted epitopes in hF.IX, AAV2-VP3, and AAV8-VP3 protein sequences based on interactions with K, D, and L loci. For the K locus, predictions were made for three common mouse haplotypes, b (C57BL/6), d (BALB/c, DBA/2), and k (C3H/HeJ). For the D and L loci, predictions were made for b and d haplotypes, respectively (Table 59). For each MHC locus and mouse genome haplotype, the top three predicted peptides are shown, using scoring from two prediction programs. SYFPEITHI predictions are based on the three-dimensional structures of class I MHC and peptides with known cleavage sites. BIMAS predictions are based on experimental results of known class I MHC and peptides. For the AAV2 and AAV8 capsid, and the hF.IX transgene, the highest scoring was for H2-Kd interactive peptides, SYFPEITHI and BIMAS. Other high-scoring peptides for binding to the H2-Ld locus were aa 170-178 (VPQYGYLTL, SEQ ID NO: 1110) in the AAV2 capsid and aa 166-174 (QPARKRLNF, SEQ ID NO:1111) in the AAV8 capsid.

TABLE 59
Summary of the predicted CTL epitopes of AAV2, AAV8, and hF.IX.

| | | | | | | |
|---|---|---|---|---|---|---|
| H2 locus | | | | | | |
| (allele) | Haplotype | Rank | AA | Sequence | SYF^{a} | BIM^{b} |

AAV2

| | | | | | | | |
|---|---|---|---|---|---|---|---|
| K | b(8-mer) | 1 | 171-178 | PQYGYLTL | 27 | 10 | SEQ ID NO: 1112 |
| | | 2 | 209-216 | FSYTFEDV | 24 | 6.6 | SEQ ID NO: 1113 |
| | | 3 | 169-176 | MVPQYGYL | 21 | 60 | SEQ ID NO: 1114 |
| | d(9-mer) | 1 | 373-381 | QYGSVSTNL^{c} | 27 | 4147 | SEQ ID NO: 1115 |
| | | 2 | 470-478 | QYSTGQVSV | 22 | 720 | SEQ ID NO: 1116 |
| | | 3 | 140-148 | VFTQSEYQL | 19 | 1152 | SEQ ID NO: 1117 |
| | k(8-mer) | 1 | 345-352 | SEKTNVDI | 22 | 500 | SEQ ID NO: 1118 |
| | | 2 | 144-151 | SEYQLPYV | 19 | 100 | SEQ ID NO: 1119 |
| | | 3 | 350-357 | VDIEKVMI | 18 | 150 | SEQ ID NO: 1120 |
| | K(9-mer) | 1 | 228-236 | LDRLMNPLI^{e} | 20 | | SEQ ID NO: 1121 |
| | | 2 | 361-369 | EEIRTTNPY^{e} | 20 | 100 | SEQ ID NO: 1122 |
| | | 3 | 407-415 | RDVYLQGPI | 19 | 100 | SEQ ID NO: 1123 |
| D | b(9-mer) | 1 | 390-398 | TADVNTQGV^{e} | 23 | 3.4 | SEQ ID NO: 1124 |
| | | 2 | 89-97 | FSPRDWQAL | 20 | 8.5 | SEQ ID NO: 1125 |
| | | 3 | 355-363 | VMITDEEEI | 20 | 8.5 | SEQ ID NO: 1126 |
| L | d (9-mer) | 1 | 170-178 | VPQYGYLIL | 25 | 292 | SEQ ID NO: 1127 |
| | | 2 | 332-330 | FPQSQCUF | 25 | 585 | SEQ ID NO: 1128 |
| | | 3 | 160-168 | SPLMGGFGL | 23 | 195 | SEQ ID NO: 1129 |

AAV8

| | | | | | | | |
|---|---|---|---|---|---|---|---|
| K | b(8-mer) | 1 | 376-383 | PQYGYLTL | 27 | 10 | SEQ ID NO: 1130 |
| | | 2 | 414-421 | FTYTFEDV | 23 | 6.6 | SEQ ID NO: 1131 |
| | | 3 | 374-381 | MIPQYGYL | 22 | 72 | SEQ ID NO: 1132 |
| | d(9-mer) | 1 | 50-58 | KYLGPFNGL^{c} | 27 | 6912 | SEQ ID NO: 1133 |
| | | 2 | $77-585 | EYGIVADNL^{d} | 23 | 2400 | SEQ ID NO: 1134 |
| | | 3 | 345-353 | VFTDSEYQL | 19 | 1152 | SEQ ID NO: 1135 |
| | k(8-mer) | 1 | 12-19 | EDNLSEGI | 24 | 400 | SEQ ID NO: 1136 |
| | | 2 | 39-46 | QDDCRGLY | 19 | | SEQ ID NO: 1137 |
| | | 3 | 349-356 | SEYQLPYY | 19 | 100 | SEQ ID NO: 1138 |
| | k(9-mer) | 1 | 11-19 | LEDNLSEGI | 21 | 100 | SEQ ID NO: 1139 |
| | | 2 | 433-451 | LDRLMNPLI^{e} | 20 | 100 | SEQ ID NO: 1140 |
| | | 3 | 566-574 | EEIKTTNPV^{e} | 20 | 100 | SEQ ID NO: 1141 |
| D | b(9-mer) | 1 | 536-544 | FFPSNGILI | 26 | 660 | SEQ ID NO: 1142 |
| | | 2 | 455-463 | GGTANTQTL | 26 | 260 | SEQ ID NO: 1143 |
| | | 3 | 495-503 | TCQNNNSNF | 22 | | SEQ ID NO: 1144 |
| L | d(9-mer) | 1 | 166-174 | QPARKRLNF | 26 | 360 | SEQ ID NO: 1145 |
| | | 2 | 365-363 | LPPFPADVF | 24 | 450 | SEQ ID NO: 1146 |
| | | 3 | 537-545 | FPSNGILIF | 24 | 390 | SEQ ID NO: 1147 |

hF.IX

| | | | | | | | |
|---|---|---|---|---|---|---|---|
| K | b(8-mer) | 1 | 337-372 | ADKEYTNI | 20 | 2 | SEQ ID NO: 1148 |
| | | 2 | 220-227 | SENDFTRV | 19 | 14 | SEQ ID NO: 1149 |
| | | 3 | 440-447 | KYQYTKY | 18 | 2.6 | SEQ ID NO: 1150 |
| | d(9-mer) | 1 | 311-319 | KYNHDIALL^{c} | 24 | 3456 | SEQ ID NO: 1151 |
| | | 2 | 351-359 | GYVSGWGRV^{d} | 22 | 1440 | SEQ ID NO: 1152 |
| | | 3 | 329-337 | SYVTPICIA | 24 | 100 | SEQ ID NO: 1153 |
| | k(8-mer) | 1 | 207-214 | AETILDNI | 22 | 1000 | SEQ ID NO: 1154 |
| | | 2 | 290-297 | TEQIKRNVI | 22 | 1000 | SEQ ID NO: 1155 |
| | | 3 | 337-344 | ADKEYTNI | 20 | 100 | SEQ ID NO: 1156 |
| | k(9-mer) | 1 | 128-136 | CELQVTQNI | 23 | 1000 | SEQ ID NO: 1157 |
| | | 2 | 9-17 | AESPQLITI | 22 | 1000 | SEQ ID NO: 1158 |
| | | 3 | 248-256 | VDAFCQQSI | 23 | 150 | SEQ ID NO: 1159 |
| D | b(9-mer) | 1 | 254-262 | GSIVNEKWI^{d} | 26 | 936 | SEQ ID NO: 1160 |
| | | 2 | 32-40 | LDHENANKI^{e} | 23 | 240 | SEQ ID NO: 1161 |
| | | 3 | 291-299 | EQKANVIRI | 21 | 60 | SEQ ID NO: 1162 |
| L | d(9-mer) | 1 | 11-19 | SPGLITICL | 22 | 100 | SEQ ID NO: 1163 |
| | | 2 | 364-352 | RSALVLQYL | 22 | 25 | SEQ ID NO: 1164 |
| | | 3 | 113-121 | NSYECWCPF | 20 | 50 | SEQ ID NO: 1165 |

^{a}Score predicted using the SYFPEITHI program.
^{b}Score predicted using the BIMAS program.
^{c}Strong immunopositive peptides.
^{d}Weak immunopositive peptdes.
^{e}Immunonegative peptides.

Accordingly, in certain embodiments the epitopes comprising the tolerogenic nanoparticles described herein comprises one or more of the epitopes shown in Table 59 and/or their corresponding nucleic acid sequences.

The prediction program SYFPEITHI accurately predict class II MHC peptide epitopes for H2k mice. The highest scoring peptide for AAV8-VP3 was aa 126-140 (LEPLGLVEEGAKTAP (SEQ ID NO:11661112)) interacting with I-Ek, yielding a a score of 28. Other high-ranking peptides include the I-Ek interactive sequences AAV 475-489 (QVSVEIEWELQKENS (SEQ ID NO:11671113)) from AAV2-VP3, with a score of 22 (Table 60). The two highest scoring I-Ek peptides for hF.IX were aa 328-342 (NSYVTPICIADKEYT (SEQ ID NO:11681114)), with a score of 24, and aa 444-460 (YTKVSRYVNWIKEKT (SEQ ID NO:11691115)), with score of 22.

TABLE 60
Summary of MHC II epitopes for
AAV2-VP3, AAV8-VP3, and hF.IX.

| | | | |
|---|---|---|---|
| MHC | AA | | |
| II | position | Sequence | Score^{a} |

AAV2

| | | | | |
|---|---|---|---|---|
| H2-E^{k} | 475-489 | QVSVEIEWELQKENS^{b} | 22 | SEQ ID NO: 1170 |
| | 336-350 | GVLIFGKQSEKTNV^{b} | 22 | SEQ ID NO: 1171 |
| H2-A^{k} | 289-303 | TSADNNNSEYSWTGA | 24 | SEQ ID NO: 1172 |
| | 309-323 | NGRDSLVNPGPAMAS | 24 | SEQ ID NO: 1173 |

AAV8

| | | | | |
|---|---|---|---|---|
| H2-E | 126-140 | LEPLGLVEEGAKTAP^{b} | 28 | SEQ ID NO: 1174 |
| | 50-64 | YKYLLGPINGUDKGEP^{d} | 28 | SEQ ID NO: 1175 |
| H2-A | 581-595 | NADNLQQONTAPQI | 26 | SEQ ID NO: 1176 |
| | 13-27 | DNLSEGIREWWALKKP | 22 | SEQ ID NO: 1177 |

Hf.IX

| | | | | |
|---|---|---|---|---|
| H2-E^{k} | 328-342 | NSYVTPICIADKEYT^{c} | 24 | SEQ ID NO: 1178 |
| | 444-460 | YTKVSRYVNWIEKEKT^{b} | 22 | SEQ ID NO: 1179 |
| | 151-165 | NKWCSCTEGYRLAE | 22 | SEQ ID NO: 1180 |
| H2-A^{k} | 312-326 | YNHDIALLELDEPLV^{c} | 24 | SEQ ID NO: 1181 |
| | 108-122 | CKDDINSYECWCPFG^{b} | 18 | SEQ ID NO: 1182 |
| | 90-104 | QYVDGDQCESNPCIN | 18 | SEQ ID NO: 1183 |

Unmarked peptides were immunonegative and not shown.
^{a}The antigenic score of each peptide was predicted using the SYI
^{b}Peptides in bold are strong imunopositive MHC II peptides.
^{c}Immunopositive.
^{d}Weak immunopositive.

Accordingly, in certain embodiments the epitopes comprising the tolerogenic nanoparticles described herein comprises one or more of the epitopes shown in Table 60 and/or the corresponding nucleic acid sequences.

The validity of epitope predictions were further corroborated by using IL-4 and IFN-γ ELISPOT assays, as well as a CTL assay. In short, the authors identified that the AAV2 capsid sequence aa 373-381, the AAV8 capsid sequence aa 50-58, and the hF.IX transgene sequence aa 311-319 can elicit CTL responses, by performing IFN-γ ELISPOT and cytotoxic killing assays. Furthermore, a strong H2k MHC II-restricted Th1 response could be elicited in C3H mice by the AAV8 capsid peptide aa 126-140 and the hF.IX peptide aa 108-122, whereas a strong Th2 response can be elicited by the AAV2 peptide aa 475-489. These results indicate that different capsid epitopes preferentially induce either a Th1 or a Th2 response and that individuals with certain MHC haplotypes are more predisposed to mounting such responses. The important take away from the study is the possibility to induce tolerogenic responses to AAV2, AAV8, or hF.IX by CD4 or CD8 T-cell activation, using specific peptide epitopes (Table 61).

TABLE 61
AAV2, AAV8 and hF.IX T cell epitopes
for use in animal models.

| No. | Epitope | Specifications | Sequence | pI | GRAVY |
|---|---|---|---|---|---|
| 1 | AAV2 capsid | 373-381 | QYGSVSTNL | 5.52 | −0.333 |
| | | | (SEQ ID NO: 1184) | | |
| 2 | AAV9 capsid | 50-58 | KYLGPFNGL | 8.59 | −0.078 |
| | | | (SEQ ID NO: 1185) | | |
| 3 | hF.IX | 311-319 | KYNHDIALL | 6.74 | −0.167 |
| | | | (SEQ ID NO: 1186) | | |
| 4 | AAV2 capsid | 475-489 | QVSVEIEWELQKENS | 4.09 | −0.947 |
| | | | (SEQ ID NO: 1187) | | |
| 5 | AAV8.capsid | 126-140 | LEPLGLVEEGAKTAP | 4.25 | 0.007 |
| | | | (SEQ ID NO: 1188) | | |
| 6 | hF.IX | 108-122 | CKDDINSYECWCPFG | 4.03 | −0.540 |
| | | | (SEQ ID NO: 1189) | | |

Accordingly, in certain embodiments the epitopes comprising the tolerogenic nanoparticles described herein comprises one or more of the epitopes shown in Table 61 and/or the corresponding nucleic acid sequences.

For the possible use of peptide therapy, all epitopes have favorable loading characteristics for PLGA nanoparticle encapsulation, with the exception of AAV8 aa 126-140. In addition, it is possible to develop mRNA vaccines, including the example in Table 63, showing a multi-epitope coding sequence that was derived by inserting the mouse transferrin receptor (aa 1-118) targeting sequence upstream of the reverse translated nucleotide sequences for the peptide sequences shown in Table 62). The premise for including both CD4 and CD8-cell epitopes stems from the fact that LSECs are capable of inducing tolerance involving both cell types.

TABLE 62
AAV2, AAV8m and hF.IX reversed translated
T cell epitopes form RN Apreparations.

| | | Specific- | |
|---|---|---|---|
| No. | Epitope | ations | Sequence |
| 1 | AAV2 capsid | 373-381 | cagtatggcagcgtgagcaccaa |
| | | | cctg |
| | | | (SEQ ID NO: 1190) |
| 2 | AAV9 capsid | 50-58 | aaatatctgggcccgtttaacgg |
| | | | cctg |
| | | | (SEQ ID NO: 1191) |
| 3 | hF.IX | 311-319 | aaatataaccatgatattgcgct |
| | | | gctg |
| | | | (SEQ ID NO: 1192) |
| 4 | AAV2 capsid | 475-489 | caggtgagcgtggaaattgaatg |
| | | | ggaactgcagaaagaaaacagc |
| | | | (SEQ ID NO: 1193) |
| 5 | AAV8.capsid | 126-140 | ctggaaccgctgggcctggtgga |
| | | | agaaggcgcgaaaaccgcgccg |
| | | | (SEQ ID NO: 1194) |
| 6 | hF.IX | 108-122 | tgcaaagatgatattaacagcta |
| | | | tgaatgctggtgcccgtttggc |
| | | | (SEQ ID NO: 1195) |

Following the addition of start and stop codons, the integrated cDNA nucleotide sequence was used for codon optimization to perform murine experimentation. The sequence will be spliced into a pTNT plasmid for mRNA expression, which will be incorporated into cationic lipid and polymer nanoparticles, as described in previous sections . . .

TABLE 63
Codon-optimized sequence for multi-epitope
delivery of tolerogenic AAV epitopes.
Complete coding sequence with mouse TfR1-118
domain (cyan), upstream of the AAVT cell
epitopes connected by cleavable linkers
(yellow and green), and with the addition of
start (purple) and stop codons (red).
Average GC content: 56.90%.

ATGATGATGGACCAGGCCAGGTCCGCCTTTTCTAATCTCTTTGGCGGAGA
GCCCTTAAGCTACACACGCTTCTCCCTGGCGAGACAAGTGGATGGAGATA
ATAGTCACGTGGAGATGAAGCTTGCTGCAGATGAAGAAGAGAATGCTGAC
AACAACATGAAAGCAAGTGTCAGAAAACCAAAGAGGTTTAATGGACGTCT
TTGTTTTGCTGCCATCGCTCTGGTGATCTTCTTCCTGATTGGCTTCATGT
CTGGCTATCTCGGCTACTGTAAACGAGTGGAGCAGAAGGAGGAGTGTGTG
AAGCTGGCAGAGACGGAGGAAACTGACAAGTCAGAGACCATGGAGACAGA
AGATGTGGGTGGAGGAAGTCGGGTTCGCCGGGGAGGGGGTGGTAGCCAGC
AGTATGGCTCTGTCTCCACCAACCTGGGGGGCGGAGGATCCCGAGTCAGG
AGAGGCGGGGGGGGGAGCAAATACCTTGGTCCTTTCAATGGCTTGGGTGG
TGGAGGGTCACGGGTCCGAAGGGGCGGGGGCGGCAGTAAGTACAACCATG
ACATAGCCCTATTGGGCGGTGGTGGCTCGCGCGTCAGGAGAGGAGGCGGC
GGATCACAAGTTTCTGTGGAAATTGAGTGGGAGCTGCAGAAAGAAAACAG
CGGGGGAGGAGGCTCCAGAGTACGGAGAGGGGGGGGGGGGTCGCTGGAGC
CTCTAGGGCTCGTAGAAGAAGGAGCCAAGACTGCCCCAGGAGGAGGTGGC
AGCAGGGTGCGCCGGGGCGGGGGTGGTAGCTGCAAGGATGACATCAACTC
TTATGAATGCTGGTGCCCCTTTGGGCGTGTTAGAAGGTGA (SEQ ID
NO: 1196)

The foregoing tolerogenic antigens and nucleic acids encoding such antigens are illustrative and non-limiting. Using the teaching provided herein, numerous other tolerogenic antigens can be incorporated into the tolerogenic nanoparticle design for the treatment and/or prophylaxis of neutralizing immune responses to viral gene vectors.

### Assessment of MHC-II Binding Interactions for Selecting Epitopes to be Included in Tolerogenic Nanoparticles

Since there is no known linkage of peanut anaphylaxis with specific HLA alleles, peptide selection for therapeutic intervention should allow promiscuous MHC-II binding to obtain widespread population coverage. The polymorphic nature of these alleles could mean that a single T cell epitope may be able to dock onto more than one major histocompatibility complex. This would mean that the number of people in the population, capable of responding to a given epitope will depend on frequency distribution of the specific HLA genotypes in the population.

MHC-II binding interactions can be assessed by the Population Coverage Calculation tool in the Immuno Epitope Database (IEDB). Accordingly, we used this tool to select peptides that exhibit promiscuous HLA interactions. While early algorithms for assessment of the HLA interactions of peanut epitopes in humans have focused on HLA-DR, we have expanded prediction making to include HLA-DQ and HLA-DP alleles that actually allows more frequent interaction with promiscuous epitope sequences.

While the time-honored approach for assessing MHC-II binding has been the use of tetramers and blocking monoclonal antibodies to assess DR, DP and DQ binding, and this is a labor intensive task that can now be substituted by in silico tools to refine the epitope selections in the IEDB and the literature. Examples of in silico tools include MHC class II binding predictions with NetMHCIIpan 2.0 (peptide length, 15; 1-mer offset), combined with information about the frequency distribution of class II alleles in, for example, the North American population. We also use in vitro MHC class II peptide reporter assays that can be performed with synthetic 15-mer peptides. For instance, Ramesh et al. (2016) supra. demonstrated how in silico epitope discovery can be used in combination with in vitro peptide-MHC binding assays, e.g., using the high throughput screening technology available from ProImmune REVEAL (ProImmune, Oxford, United Kingdom). This technology also allows assessment of the conformational change in the MHC class II molecules upon peptide docking. By using this technology in combination with cellular proliferation and cytokine assays, it was possible to identify 4 tolerogenic “vaccine” candidates among the 36 Ara h 1 peptides described above. The same approach can also be implemented to identify other tolerogenic “vaccine” epitope sequences for other allergens and autoimmune antigens.

### Immunomodulators.

In various embodiments the tolerogenic activity of the nanoparticles described herein can be enhanced by incorporation of one or more immune modulators (e.g., immune suppressors. It was discovered that incorporation of an immune modulator (e.g., an immune inhibitor such as rapamycin and/or a rapamycin analog) can significantly enhance the activity of the tolerogenic nanoparticles described herein. Not only does rapamycin offer the possibility of reprogramming the targeted antigen presenting cells in the liver, but is also known to promote Fox P3 expression on Tregs, in addition to expanding Treg populations.

Accordingly, in certain illustrative, but non-limiting embodiments, the immune modulator(s) comprise rapamycin (sirolimus). In certain illustrative, but non-limiting embodiments, the immune modulator(s) comprise one or rapamycin analogs including, but not limited to rapamycin analogs selected from the group consisting of temsirolimus, everolimus, and ridaforolimus. These rapamycin analogs (rapalogs) are illustrative and non-limiting. Numerous other rapalogs are known to those of skill in the art. Thus, for example, the preparation of mono- and di-ester derivatives of rapamycin is described in WO 92/005179, 27-oximes of rapamycin are described in EPO Patent Application No: EPO 467606, 42-oxo analog of rapamycin are described in U.S. Pat. No. 5,023,262, bicyclic rapamycins are described in U.S. Pat. No. 5,120,725, rapamycin dimers are described in U.S. Pat. No. 5,120,727, silyl ethers of rapamycin are described in U.S. Pat. No. 5,120,842, and rapamycin arylsulfonates and sulfamates are described in U.S. Pat. No. 5,177,203. The foregoing applications are incorporated herein by reference for the rapamycin analogs described therein.

Using the teaching provided herein, numerous other immunomodulators can be incorporated into the tolerogenic nanoparticles described herein. These include but are not limited to immunomodulators that specifically strengthen the duration and immune suppressive effects of regulatory T cells, in addition to the effect that they may exert in reprogramming antigen presenting cells towards a tolerogenic response. Additional immunomodulators that can modify the quality of the Treg response, in addition to rapamycin, can be incorporated, e.g., as described below are shown below in Table 64. Such immunomodulators include but are not limited to All-trans Retinoic Acid (ATRA) and nucleoside DNA methyl transferase inhibitors (e.g., 5-Azacitidine). Mechanisms of action are described in Table 64.

TABLE 64
Illustrative immunomodulators that can modify the
Treg response and their mechanisms of action.

| Agent | Mechanism of action | Treg impact |
|---|---|---|
| Rapamycin | mTOR inhibitor | Promotes FoxP3 expression; |
| | | Expands iTregs populations; |
| | | Synergize with ATRA |
| | | to enhance Treg |
| | | immune suppression |
| All-trans | Vitamin A | Enhances TGF-β induced Treg |
| Retinoic | derivative | induction; Increases CCR9 |
| Acid (ATRA) | (tretinoin) | expression on Tregs to home to |
| | operating | inflammatory sites in mucosal |
| | via nuclear | tissues such as the |
| | receptors | GALT, binding to |
| | | the α₄β₇ integrin |
| 5-Azacitidine | Nucleoside | Enhanced FoxP3 expression |
| Decitabine | DNA methyl | (demethylation of |
| | transferase | TSDR promoter region; |
| | inhibitors | Treg expansion and |
| | | improved control of |
| | | chronic GVHD |

The foregoing immunomodulators are illustrative and non-limiting. Using the teaching provided herein, numerous other immunomodulators will be available to one of skill in the art for incorporation into the tolerogenic nanoparticles described herein.

### Pharmaceutical Formulations and Administration.

In various embodiments tolerogenic nanoparticles are provided for preventing or ameliorating an autoreactive condition (e.g., an allergic condition, immune-mediated adverse events to therapeutics, autoimmune disease, transplant rejection, and the like).

In certain embodiments the tolerogenic nanoparticles are provided for administration as a pharmaceutical formulation. In certain embodiments such formulations comprise the tolerogenic nanoparticles and a pharmaceutically acceptable carrier. In certain embodiments such pharmaceutical formation can be administered with and/or can incorporate one or more modulators of the immune system (e.g., immunosuppressants such as rapamycin, fujimycin, methotrexate, etc.). The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art.

In certain embodiments the tolerogenic nanoparticles described herein can be formulated for parenteral administration, e.g., formulated for injection via the intravenous, intramuscular, subcutaneous, intrathecal or even intraperitoneal routes. Methods of preparing an aqueous composition that contains tolerogenic nanoparticles described herein will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Illustrative pharmaceutical forms suitable for injectable use include sterile aqueous solutions or dispersions; formulations including various oils and/or propylene glycol; and sterile powders for the extemporaneous preparation of sterile injectable solutions or dispersions. Typically the form must be sterile and must be fluid to the extent that it may be easily injected. It also should be stable under the conditions of manufacture and storage and is desirably preserved against the contaminating action of microorganisms, such as bacteria and fungi.

In certain embodiments the active pharmaceutical ingredients in tolerogenic nanoparticles described herein can be formulated into a neutral or salt form. Pharmaceutically acceptable salts, include the acid addition salts (formed with the free amino groups of the protein) and which are formed with, e.g., inorganic acids such as, for example, hydrochloric or phosphoric acids, or such organic acids as acetic, oxalic, tartaric, mandelic, and the like. Salts formed with free carboxyl groups can also be derived from inorganic bases such as, for example, sodium, potassium, ammonium, calcium, or ferric hydroxides, and such organic bases as isopropylamine, trimethylamine, histidine, procaine and the like.

In certain embodiments the carrier also can be a solvent or dispersion medium containing, for example, water, ethanol, polyol (for example, glycerol, propylene glycol, and liquid polyethylene glycol, and the like), suitable mixtures thereof, and various oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating the required amount of tolerogenic nanoparticles described herein in the appropriate solvent with various of the other ingredients enumerated above, as required, followed, e.g., by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, illustrative methods of preparation are vacuum-drying and freeze-drying techniques, which yield a powder of the active ingredient, plus any additional desired ingredient from a previously sterile-filtered solution thereof.

In certain embodiments the tolerogenic nanoparticles described herein are formulated for oral administration, rectal administration, administration via inhalation, and the like. Methods of formulation compositions for such routes of administration are well known to those of skill in the art.

Administration of the tolerogenic nanoparticles described herein or formulations thereon will typically be via any common route. This includes, but is not limited to orthotopic, intradermal, subcutaneous, intramuscular, intraperitoneal, or intravenous injection, oral administration, inhalation, and the like. Methods of formulation compounds for administration via inhalation are described, for example, in U.S. Pat. No. 6,651,655.

In various embodiments, an effective amount of the tolerogenic nanoparticles described herein (e.g., a therapeutically effective or prophylactically effective amount) can be based on the desired objective. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result (e.g., immune tolerance and/or reduction in immune response) desired.

In certain embodiments precise amounts of the composition also depend on the judgment of the practitioner and are peculiar to each individual. Factors affecting dose include physical and clinical state of the subject, route of administration, intended goal of treatment (e.g., alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### Uses of the Tolerogenic Nanoparticles.

It will be recognized that the tolerogenic nanoparticles described herein can be used to treat or ameliorate a number of immune-mediated or autoimmune diseases, e.g., diabetes, graft rejection, etc. In certain embodiments the tolerogenic nanoparticle described herein are used for the treatment and/or prophylaxis of a peanut allergy in a mammal, or for the treatment and/or prophylaxis of a dust mite allergy in a mammal, or for suppressing or preventing an immune response to adalimumab in a mammal, or for the treatment and/or prophylaxis of type I diabetes in a mammal, or for the treatment and/or prophylaxis of lupus in a mammal, or for the suppressing or preventing an immune response to an AAV viral vector in a mammal (e.g., in an an AAV-based gene therapy).

The foregoing pathologies and tolerogenic antigens are illustrative and non-limiting. Using the teaching provided herein numerous other tolerogenic antigens can be incorporated into the tolerogenic nanoparticles described herein and used for the treatment and/or prophylaxis of a wide range of conditions.

### Kits Comprising Tolerogenic Nanoparticles.

In various embodiments kits are provided for inducing immune tolerance (e.g., epitope-specific immune tolerance) and/or for reducing an immune response to a particular antigen. In various embodiments the kits comprise a container containing one or more of the tolerogenic nanoparticles described herein.

In addition, the kits optionally include labeling and/or instructional materials providing directions (e.g., protocols) for the use of the tolerogenic nanoparticles described herein, e.g., alone or in with, e.g., various immune suppressants, for the treatment or prophylaxis of various allergic and/or autoimmune, or transplant-related pathologies.

While the instructional materials in the various kits typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Use of Polymeric Nanoparticle Platform Targeting the Liver To Induce Treg-Mediated Antigen-Specific Immune Tolerance in a Pulmonary Allergen Sensitization Model

Nanoparticles (NPs) can be used to accomplish antigen-specific immune tolerance in allergic and autoimmune diseases. The available options for custom-designing tolerogenic NPs include the use of nanocarriers that introduce antigens into natural tolerogenic environments, such as the liver, where antigen presentation promotes tolerance to self- or foreign antigens. In this example, we demonstrate the engineering of a biodegradable polymeric poly(lactic-co-glycolic acid) (PLGA) nanocarrier for the selective delivery of the murine allergen, ovalbumin (OVA), to the liver. This was accomplished by developing a series of NPs in the 200-300 nm size range as well as decorating particle surfaces with ligands that target scavenger and mannose receptors on liver sinusoidal endothelial cells (LSECs). LSECs represent a major antigen-presenting cell type in the liver capable of generating regulatory T-cells (Tregs). In vitro exposure of LSECs to NP^{OVA} induced abundant TGF-β, IL-4, and IL-10 production, which was further increased by surface ligands. Animal experiments showed that, in the chosen size range, NP^{OVA} was almost exclusively delivered to the liver, where the colocalization of fluorescent-labeled particles with LSECs could be seen to increase by surface ligand decoration. Moreover, prophylactic treatment with NP^{OVA} in OVA-sensitized and challenged animals (aerosolized inhalation) could be seen to significantly suppress anti-OVA IgE responses, airway eosinophilia, and TH2 cytokine production in the bronchoalveolar lavage fluid. The suppression of allergic airway inflammation was further enhanced by attachment of surface ligands, particularly for particles decorated with the ApoB peptide, which induced high levels of TGF-β production in the lung along with the appearance of Foxp3⁺ Tregs. The ApoB-peptide-coated NPs could also interfere in allergic airway inflammation when delivered post-sensitization. The significance of these findings is that liver and LSEC targeting PLGA NPs could be used for therapy of allergic airway disease, in addition to the potential of using their tolerogenic effects for other disease applications.

### Results

### Synthesis of the PLGA NP Platform for OVA Delivery

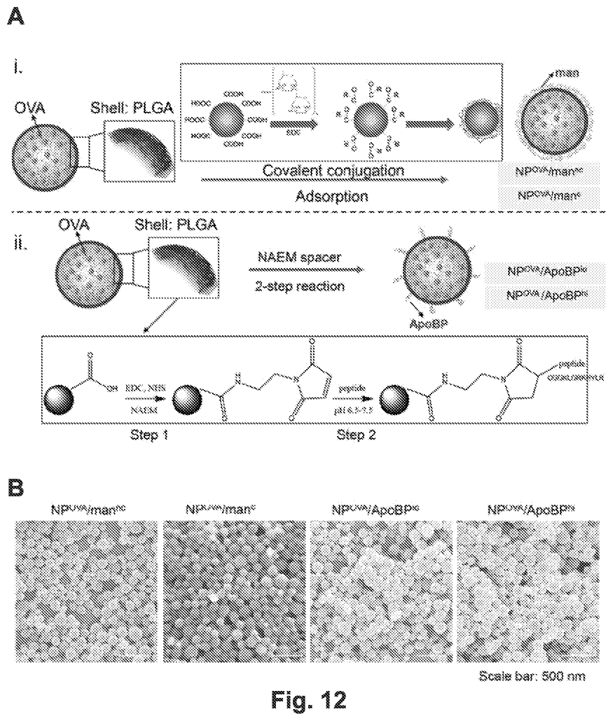

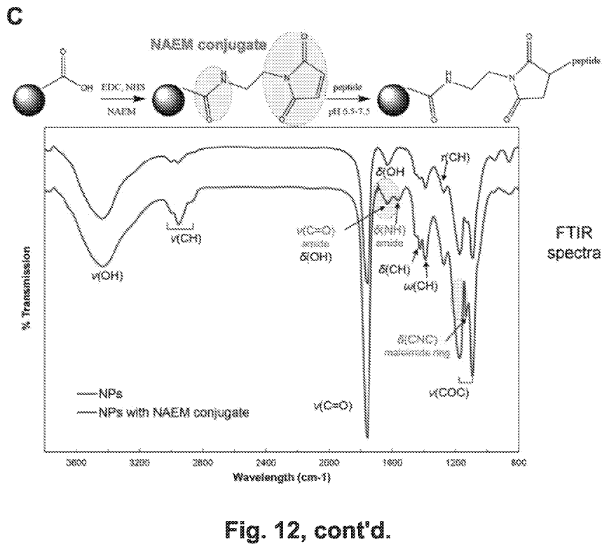

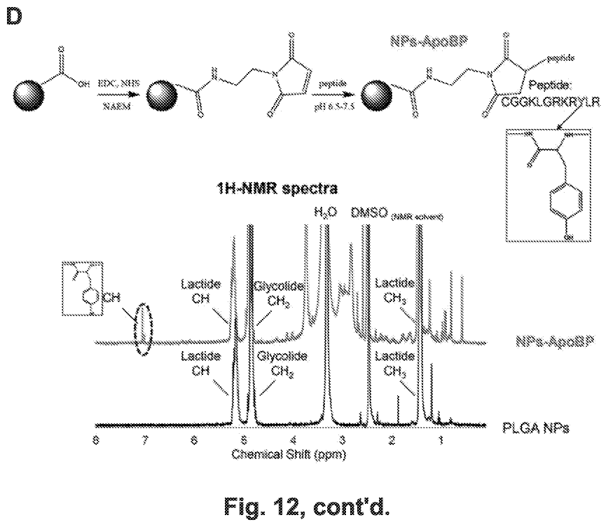

The PLGA polymer was selected for NP synthesis because it is biodegradable, biocompatible, and FDA-approved for drug delivery (see e.g., Prickett et al. (2013) Clin. Exp. Allergy, 43 (6): 684-697; Pascal et al. (2013) Clin. Exp. Allergy, 43 (1): 116-127; Ramesh et al. (2016) J. Allergy Clin. Immunol. 137 (6): 1764-1771.e4). A double-emulsion method, involving solvent evaporation, was used to fabricate PLGA NPs entrapping 50 μg/mg OVA. Because most NPs in the size range above 500 nm are phagocytosed by KC in the liver or macrophages in the liver and spleen (see, e.g,. Patrawala et al. (2020) Curr. Allergy Asthma Rep. 20 (5): 14; Tiegs & Lohse (2010) J. Autoimmun. 34 (1): 1-6), our aim was to develop smaller, easy-to-synthesize particles using a water-in-oil-in-water (w/o/w) emulsion method (FIG. 12, panel A). We settled on the synthesis of a primary particle size of 230 nm (FIG. 12, panel B, and Table 65), which yielded a size range of 246-297 nm following the attachment of surface ligands that could target mannose and stabilin receptors on liver cells. 42.43 Mannan (man) was selected to target the mannose receptor, whereas an ApoB peptide (ApoBP), RLYRKRGLK (SEQ ID NO:19), was used as a ligand for stabilin receptors (see, e.g., Patrawala et al. (2020) supra; O'Hehir et al. (2016) Curr. Allergy Asthma Rep. 16 (2): 14; Dorofeeva et al. (2021) Allergy, 76 (1): 131-149; Worm et al. (2011) J. Allergy Clin. Immunol. 127 (1): 89-97, 97.e1-14). Whereas the stabilin receptors are exclusively expressed on LSECs, the mannose receptor is also present in low abundance on the surface of KCs. Mannan attachment to PLGA particle surfaces was achieved by physical adsorption or using a one-step covalent conjugation method to yield NP^{OVA}/man1° C. and NP^{OVA}/mane NP, respectively. For the attachment of ApoBP peptide, lower and higher molar ratios were used for conjugation to the polymer backbone by a two-step procedure, making use of a NAEM spacer; these particles were designated as NP^{OVA}/ApoBP^{lo} and NP^{OVA}/ApoBP^{hi}, respectively (FIG. 12, panel A).

TABLE 65
Comparison of Different NP Formulations for Hydrodynamic Size,
Zeta-Potential, OVA Content, Ligand Content, and Molar Ratios.

| | | | | OVA | Ligand | |
|---|---|---|---|---|---|---|
| | Hydrodynamic | Polydispersity | Zeta- | content | content | Mol % |
| | size | index | potential | (μg/mg | (μg/mg | of |
| Nanoparticle | (nm) | (PDI) | (mV) | NPs) | NPs) | PLGA |
| NP only | 231.2 ± | 0.096 | −42.55 ± | NA | NA | NA |
| | 2.17 | | 2.99 | | | |
| NP^{OVA} | 246.5 ± | 0.105 | −44.37 ± | 51.61 ± | NA | NA |
| | 3.01 | | 2.81 | 2.32 | | |
| NP^{OVA}/man^{nc} | 279.5 ± | 0.121 | −51.63 ± | 50.31 ± | 139 ± | NA |
| | 2.74 | | 4.05 | 3.52 | 21 | |
| NP^{OVA}/man^{c} | 297.2 ± | 0.113 | −54.82 ± | 50.98 ± | 346 ± | NA |
| | 2.98 | | 5.18 | 3.88 | 52 | |
| NP^{OVA}/ApoBP^{lo} | 268.8 ± | 0.109 | −8.63 ± | 50.77 ± | 6.86 ± | 2.8 |
| | 4.96 | | 1.38 | 3.08 | 0.89 | |
| NP^{OVA}/ApoBP^{hi} | 270.8 ± | 0.113 | −4.56 ± | 50.12 ± | 12.96 ± | 5.3 |
| | 4.96 | | 2.25 | 2.18 | 0.77 | |

The NP formulations were characterized for hydrodynamic size, zeta-potential, OVA content, and ligand density as outlined in Table 65. The physicochemical characterization demonstrated a mean hydrodynamic diameter of 230-290 nm, with the larger size being due to the attachment of surface ligands. NP without OVA (designated NP-only) or encapsulating OVA only (NP^{OVA}) exhibited a negative surface charge in water, with zeta-potentials of −42.55 and −44.37 mV, respectively. Mannan attachment increased the negative charge, whereas ApoBP conjugation brought the zeta-potential to −8.63 and −4.56 mV for NP^{OVA}/ApoBP^{lo} and NP^{OVA}/ApoBP^{hi}, respectively.

The relative abundance of mannan incorporation was assessed by calculating the difference between the total amount offered for conjugation versus the amount recovered in the supernatant. Mannan quantity was determined by a colorimetric method (using phenol-sulfuric acid (see Dorofeeva et al. (2021) supra) to demonstrate a mannan content of 346±52 μg per mg of NPs after covalent attachment, whereas physical adsorption amounted to 139±21 μg/mg. The ApoBP conjugation to the NAEM spacer was determined by Fourier transform infrared spectroscopy, which demonstrated the presence of two amide bonds (stretching peaks at ˜1600 cm-1) as well as a maleimide ring (vibration peak at ˜1100 cm-1) (FIG. 12, panel C). We also assessed the proton NMR spectra of the pristine particles, showing the presence of the lactide (—CH and —CH₃) and glycolide (—CH₂) peaks in the PLGA backbone, as well as the appearance of an ApoBP tyrosine peak (˜7 ppm) in conjugated particles (FIG. 12, panel D). The abundance of peptide conjugation was assessed by a microBCA assay and a nanodrop method. Both methods showed a peptide quantity of ˜2.8 and 5.3 mol % in the completed PLGA construct.

Scanning electron microscopy was performed to show the morphology of the spherical NPs, which were demonstrated to be of uniform size (FIG. 12, panel B). The biocompatibility of these materials in LSECs and Kupffer cells was assessed using an ATP assay, following particle addition at concentrations ranging from 25 to 300 μg/mL. No evidence of cytotoxicity was observed.

### Materials and Methods.

### Reagents.

The PLGA formulation obtained from Sigma (St. Louis, MO) has a lactide/glycolid molar ratio of 50:50, a viscosity of 0.45-0.60 dL/g, and includes a premixed content of ˜5 kDa PEG. Ovalbumin (OVA), dichloromethane, sodium cholate (used as a stabilizer in the outer water phase), mannan (mw 35-60 kDa), 1-ethyl-3-(3-(dimethylamino)propyl) carbodiimide (EDC), FITC I, N-hydroxysuccinimide (NHS), and N-(2-aminoethyl) maleimide (NAEM) were purchased from Sigma. The immortalized mouse hepatic sinusoidal endothelial cells-SV40 (LSEC), Prigrow medium, and flasks for growing LSECs were purchased from Applied Biological Materials (Vancouver, BC, Canada). The mouse Kupffer cell line, KUP5, was purchased from RIKEN Cell Bank (Japan). The ATPlite luminescence assay kit was purchased from PerkinElmer (Santa Clara, CA). Hoechst 33342, DyLight680 NHS-Ester, and the isolectin GS-IB4 were purchased from Thermo Fisher Scientific (Waltham, MA). The ApoB peptide (ApoBP), RLYRKRGLK (SEQ ID NO:19), containing a GGC tag was synthesized by Biomatik (Cambridge, Ontario, Canada). ELISA kits for the measurement of murine TGF-β, IL-4, IL-10, IL-5, IL-13, TNF-α, IL-6, IFN-γ, and IL-1β were purchased from R&D (Minneapolis, MN). The horseradish peroxidase (HRP)-conjugated goat anti-mouse secondary antibody for detection of IgG2a (A-10685) and IgE (PA1-84764) were purchased from Invitrogen (Waltham, MA). The secondary antibody for detection of IgG1 (ab97240) was from Abcam (Cambridge, MA). The 3,3′,5,5′-tetramethylbenzidine (TMB) substrate kit was purchased from BD Biosciences (San Jose, CA).

### Fabrication and Characterization of the LSEC-Targeting NPs.

Pristine PLGA NPs were fabricated using a double-emulsion, w/o/w method, combined with solvent evaporation. Two hundred milligrams of PLGA was dissolved in 10 mL of dichloromethane. Thirty milligrams of an OVA solution (1 mL) was added to the PLGA solution to form the primary emulsion (w/o), which was sonicated for 40 s, using a probe sonicator that delivers a power output of 60 W and a 4/4 s on/off working pulse. The primary emulsion was poured into 60 mL of 1% sodium cholate solution, and the mixture was sonicated for 2 min and added into 90 mL of 0.5% sodium cholate solution. The double emulsion was stirred overnight to allow the evaporation of dichloromethane. The mixture was centrifuged and washed using DI water five times at 10000g for 10 min to remove the non-encapsulated OVA, before suspension in DI water or PBS, as indicated.

Mannan attachment to the particle surface was achieved either through physical adsorption or covalent attachment. For physical adsorption, freeze-dried NPs (10 mg) were mixed with mannan (20 mg in 2 mL of PBS, pH 5.0) and stirred overnight at room temperature. The NPs were collected and washed to remove excess mannan by centrifugation at 35000g for 15 min. Conjugation chemistry was performed using the COOH-terminus of PLGA for covalent attachment of mannan. Briefly, mannan, EDC, and sulfo-NHS were added to freeze-dried NPs [n (man)/n (COOH)/n (EDC)=0.4:1:10] and dispersed in PBS (2 mL, pH 5.0). The mixture was stirred overnight at room temperature. The NPs were collected, and the excess mannan was removed by centrifugation (35000 g, 15 min). The acquired NPs were designated as NP^{OVA}/man^{C} and NP^{OVA}/man^{nc}.

For peptide conjugation, ApoBP was conjugated to NP^{OVA} by a two-step reaction, using a N-(2-aminoethyl) maleimide (NAEM) spacer. The COOH-terminal PLGA groups in the NPs were attached to the succinimidyl ester, using EDC (n (COOH)/n (EDC)=1:10) and NHS. NAEM was subsequently added (n (COOH)/n (NAEM)=3:5), and the mixture stirred for 2 h to decorate the particle surfaces with NAEM (NPs-NAEM). To remove the excess NAEM, NP^{OVA}/NAEM was purified by spinning at 35000g for 15 min. The ApoBP solution, containing a cysteine tag at the N-terminal end, was added to the suspension and stirred for an additional 2 h. This allowed the maleimide group on NAEM to react with the cysteine sulfhydryl group to form a stable thioether bond. The final product was washed to remove excess reactants. Two doses of ApoBP were used (10 or 20 mg/mL) to generate NPs with different ligand density, which were designated as NP^{OVA}/ApoBP^{lo} and NP^{OVA}/ApoBp^{hi}.

The purified NPs were fully characterized using dynamic light scattering to determine particle size and surface charge, and scanning electron microscopy was performed to visualize particle morphology. The microBCA assay was used to detect OVA loading capacity and the conjugation efficiency of the peptide ligand, whereas the phenol-sulfuric acid method was used to determine mannan concentration. The endotoxin level was verified by a chromogenic LAL assay.

### Cell Culture.

LSECs were grown in Prigrow medium, supplemented with 10% fetal bovine serum (FBS, Gemini, Sacramento, CA) and 100 U/mL/100 μg/mL of penicillin-streptomycin (Gibco, Waltham, MA). KUP5 cells were grown in high-glucose Dulbecco's modified Eagle medium (DMEM) supplemented with 10% FBS, 250 μM 1-thioglycerol, 10 μg/mL bovine insulin, and 100 U/mL/100 μg/mL of penicillin-streptomycin.

### Determination of NP Cytotoxicity.

Cytotoxicity assays were performed in LSECs and KUP5 cells using the ATP assay. Following the exposure of the cells to NPs at different concentrations for 24 h in a 96-well plate, the cell culture medium was replaced with an ATP solution. After centrifugation in a microplate centrifuge, 100 μL of each supernatant was removed to determine the luminescence intensity in a SpectraMax M5 microplate reader (Molecular Devices, Sunnyvale, CA).

### Example 2

### Tolerogenic Nanoparticles Can Induce Epitope-Specific Tolerance In Animal Ovalbumin (OVA) Allergic Sensitization Mode

We have demonstrated that targeting of liver sinusoidal endothelial cells (LSECs) with ApoB-decorated PLGA nanoparticles that encapsulate the intact protein, ovalbumin (OVA), can induce OVA specific immune tolerance in mice (see Example 1, above). Integral to understanding this form of immune tolerance is that the nanocarrier induced OVA-specific Tregs that target epitope-specific helper T cells that respond to specific OVA epitopes.

In this current study, we hypothesized that it should be possible to demonstrate epitope-specific OVA induced immune tolerance, for example, using what is known as an OT II (OVA₃₂₃₋₃₃₉) peptide, which is specifically presented by MHC II molecules that activate epitope-specific CD4⁺ T helper cells. In order to investigate the hypothesis that we can induce epitope-specific immune tolerance in animals, we purchased both OT I (OVA₂₅₇₋₂₆₄) and OT II (OVA₃₂₃₋₃₃₉) peptide for encapsulation into ApoB-peptide decorated PLGA nanoparticles for administration to OT II mice, which selectively express MHC II molecules for expression of OVA₃₂₃₋₃₃₉. In contrast to the OT II peptide, the OT I peptide is presented by MHC I, which is not expressed by OT II mice. The effect of nanoparticles encapsulating both the OT I and OT II peptides were subsequently compared for their effects on the generation of allergic inflammation in OT II mice, which were sensitized against whole OVA protein, as described in Example 1. The comparison also included a group of mice receiving PLGA nanoparticles encapsulating whole OVA, as demonstrated in Example 1.

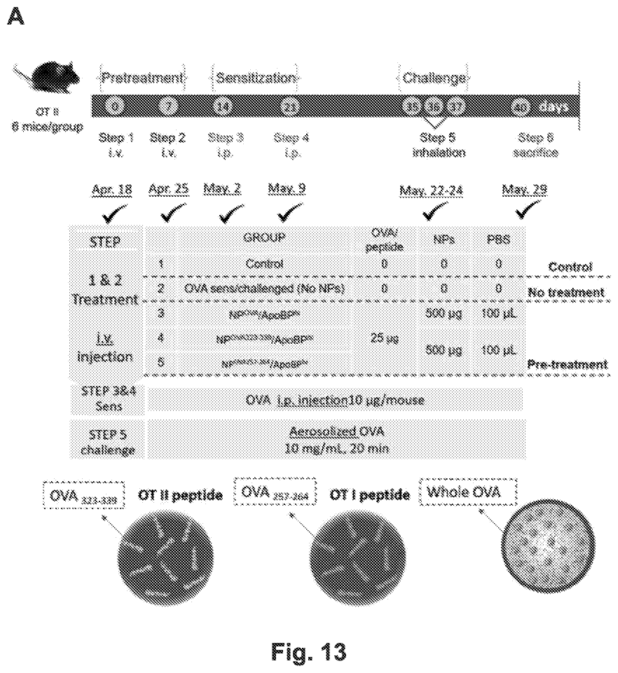

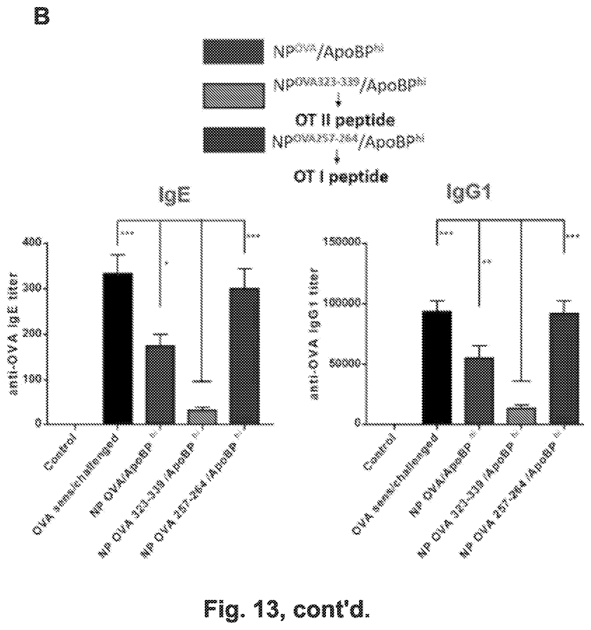

Prophylactic treatment with NP^{OT II} in OVA-sensitized and challenged animals (aerosolized inhalation) (see, FIG. 13, panel A) could be seen to significantly suppress anti-OVA IgE and IgG1 (see, FIG. 13, panel B) responses, as well as TH2-mediated eosinophilic airway inflammation. These effects were also accompanied by abundant TGF-β production in the BALF.

The immunosuppressive effects were also seen for NPs encapsulating intact OVA, but not with the same degree of robustness as NP^{OTII}. However, no tolerogenic effects were seen for NP^{OTI}. These findings demonstrate that the designed NP platform is capable of inducing antigenic epitope-specific immune tolerance, with the prediction making that the nanoparticle platform will be useful for tolerization against a long list of epitopes involved in allergic and autoimmune disease, such as type I diabetes mellitus, autoimmune disease, immunological mediated drug and treatment events, etc.

### Example 3

Use of a Liver-targeting Tolerogenic Nanoparticle Platform to Intervene in Peanut-induced anaphylaxis through delivery of an Ara h2 T-cell Epitope

Food allergy affects 15 million people in the United States, including 8% of children. Of particular concern is the steady rise in the prevalence of peanut allergy, which is responsible for the highest number of life-threatening food reactions, including anaphylaxis (see, e.g., Finkelman (2010) supra; Wang & Sampson (2007) supra; Sicherer & Sampson (2018) supra; Branum & Lukacs (2009) supra; Bock et al. (2001) supra; Boyce et al. (2010) supra). Despite promising results from clinical trials for oral or sublingual immunotherapy with peanut extracts, these interventions can still lead to adverse outcomes, and lack of tolerance (see, e.g., Varshney et al. (2011) supra; Chin et al. (2013) supra; Nurmatov et al. supra; Sheikh & Burks (2013) supra). Currently, there is only one FDA-approved oral immunotherapy treatment platform for peanut allergy, Palforzia, which is comprised of a powdered CPPE for oral desensitization therapy (see, e.g., Dougherty et al. (2021) supra; Vickery et al. (2018) supra; Santos et al. (2020) supra; Mullard (2020) supra). However, the shortcomings include the need for daily maintenance therapy, the occurrence of allergic side effects (including life-threatening anaphylaxis), and the lack of evidence that long-term tolerance can be accomplished (see, e.g., Vickery et al. (2018) supra; Mullard (2020) supra; Patrawala et al. (2020) supra; Wasserman et al. (2021) supra).

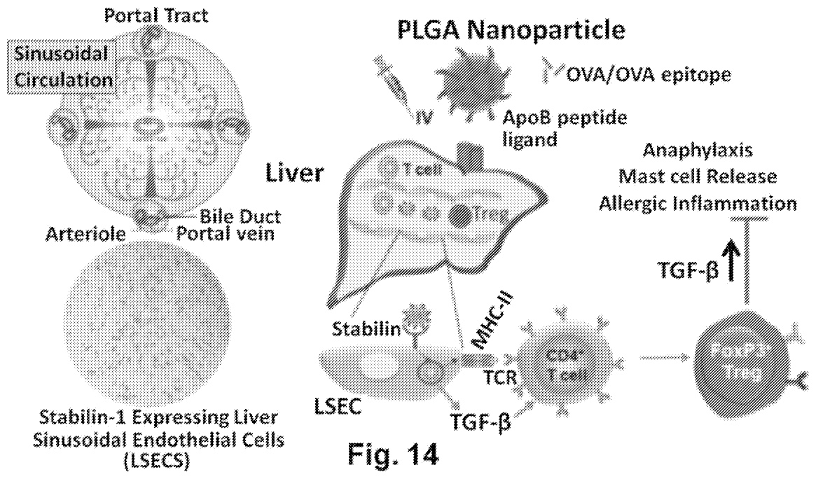

We have recently demonstrated that biocompatible and biodegradable ˜200 nm poly(lactide-co-glycolide acid) (PLGA) nanoparticles can be used to target the liver for reduction of systemic tolerance to the egg white allergen, ovalbumin (OVA), prophylactically and therapeutically (FIG. 14) (Liu et al. (2019) supra; Liu et al. (2021) supra). The liver is an immune-privileged organ capable of generating tolerogenic responses to food allergens (see, e.g., Tiegs & Lohse (2010) supra; Vickery et al. (2011) supra). While a variety of liver cell types (Kupffer cells, sinusoidal endothelial cells, hepatocytes, etc.) contribute to establishing a natural tolerogenic environment, liver sinusoidal endothelial cells (LSECs) are particularly endowed with the ability to act as antigen presenting cells (APC), capable of generating regulatory T-cells (Tregs) (see, e.g., Knolle & Wohlleber (2016) supra; Crispe, (2011) supra; Horst et al. (2016) supra). This includes a role for LSEC scavenger receptors, which are engaged in the endocytosis of foreign or damaged proteins and particulate matter that are either removed or presented by major histocompatibility type II (MHC-II) gene products to naïve T-cells, capable of generating antigen-specific Foxp3⁺ Tregs (see, e.g., Knolle & Wohlleber (2016) supra; Horst et al. (2016) supra; Carambia et al. supra; Carambia et al. (2015) supra). Thus, by using covalent attachment of a scavenger receptor Apo B100 peptide (Apo-BP) to the PLGA surface, we have successfully constructed LSEC targeting nanoparticles, allowing delivery of intact OVA or a MHC-II binding (“OT-II”) peptide fragment (amino acids 323-339) to generate Foxp3⁺ T-cells, capable of suppressing allergic airway inflammation and anaphylaxis in OVA inhalation or oral challenge models (FIG. 14) (Liu et al. (2019) supra; Liu et al. (2021) supra). Similar response outcomes could not be achieved by a MHC-I binding OVA peptide (OT-I), confirming the importance of MHC-II docking in the design of T-cell epitopes that can be incorporated as antigenic cargo in tolerogenic nanoparticles for therapeutic intervention into food allergy (Liu et al. (2021) supra).

In the current study we asked whether our tolerogenic nanoparticle platform could be used to intervene in anaphylaxis by crude peanut protein extract (CPPE) in an oral challenge model. We hypothesized that selection of dominant T-cell epitopes from Ara h2, the major conglutin playing a role in peanut allergy among 17 peanut proteins, could provide tolerogenic nanoparticle cargo for accomplishing durable suppression of allergic inflammation, mast cell release and anaphylactic reactions triggered by CPPE (Hemmings et al. (2020) supra). Use of the Immune Epitope Database (IEDB) for initial selection of murine MHC-II interactive Ara h 2 epitopes, which were further refined through the selection of peptide characteristics to ensure good nanoparticles encapsulation efficacy, yielded a dominant epitope sequence (amino acids 59-73) that could provide linked suppression of the anaphylaxis response to CPPE. Prophylaxis was shown to be effective for at least two months. Controls included purified Ara h2 protein, which was moderately effective, and a predicted Ara h2 IgE binding sequence that was ineffective. We further demonstrated that the response to the purified Ara h 2 protein and its dominant epitope was successful in generating TGF-β producing CD4⁺CD25⁺FoxP3⁺ Tregs. All considered, these results demonstrate the therapeutic potential of a dominant Ara h2 T-cell epitope for durable control of anaphylaxis induced by a crude peanut extract.

### Methods

Antigen cargo selection and synthesis of liver-targeting PLGA nanoparticles

The NIAID Immune Epitope Database (IEDB) and Analysis Resource was used for entering the complete Ara h2 amino acid (aa) sequence, and then selecting 15-mer peptide candidates with the highest binding affinity for murine MHC-II alleles (H2-IAb, H2-IEd and H2-IAd). Further peptide selection to achieve optimal encapsulation efficacy in poly (lactide co-glycolic acid) (PLGA) nanoparticles, used an online tool (//web.expasy.org/protparam/) to calculate “grand average hydropathy values” (GRAVY). GRAVY considers peptide solubility and surface charge, with positive values indicating hydrophobic and negative values indicating hydrophilic compositions. Hydrophilic sequences with an isoelectric point (p1) deviating from 7.4 are easier to load.

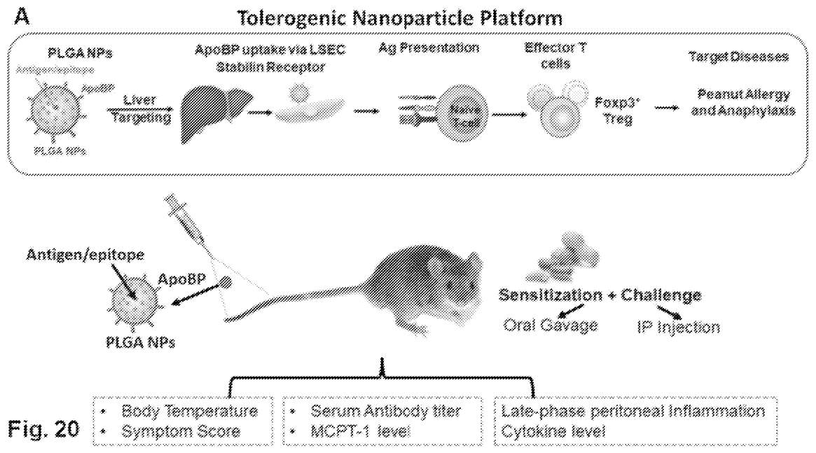

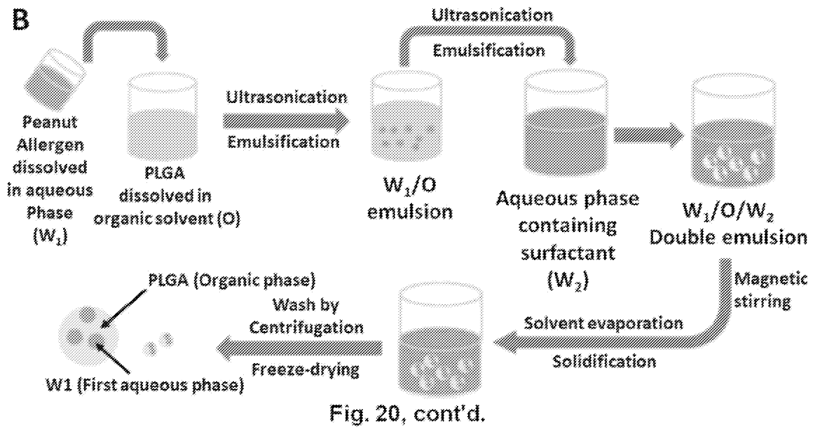

Nanoparticles were synthesized using the double-emulsion (w/o/w) method combined with solvent evaporation, as previously described and further elucidated in the Online Repository (FIG. 20, panel B) (Liu et al. (2019) supra; Liu et al. (2021) supra). Particles were separated and washed by centrifugation (15,000 g, 10 min). Hydrodynamic size and surface potential were measured using dynamic light scattering (DLS), with visualization of the particle size and morphology by scanning electron microscopy (SEM). Covalent surface conjugation of the stabilin-1 binding Apo-B peptide sequence (RLYRKRGLK (SEQ ID NO:1129), containing a GGC tag) was performed as previously reported. In addition to loading of T-cell epitopes, we also selected an IgE binding peptide for encapsulation, in addition to the synthesis of nanoparticles that incorporate CPPE as well as a purified Ara h2 protein (Indoor Biotechnologies). The CPPE was prepared as described in the Online Repository section. Allergen loading capacity, including for CPPE, purified Ara h2 and its epitopes, as well as the conjugation efficacy of the ApoB ligand was determined by bicinchoninic acid assay (BCA assay).

### Assessment of Tolerogenic PLGA Nanoparticles Effects in an Oral Anaphylaxis Model

Six-week-old female C3H/HeJ mice purchased from Jackson Laboratory (Bar Harbor, ME) were maintained in pathogen-free facilities at the Division of Laboratory Animal Medicine at UCLA. Animal experimentation followed the guidelines established by the National Society for Medical Research, as in “Principles of Laboratory Animal Care”.

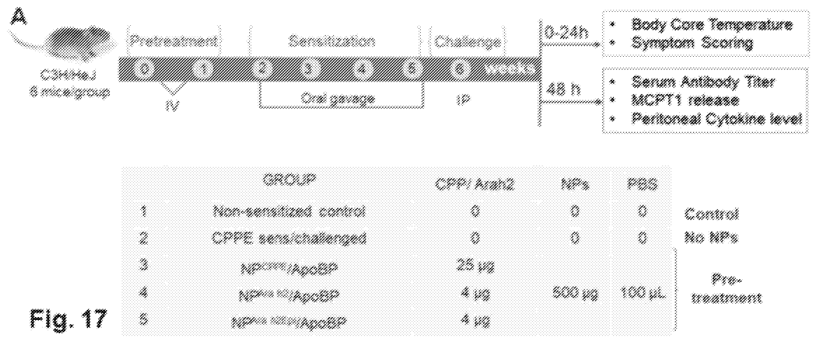

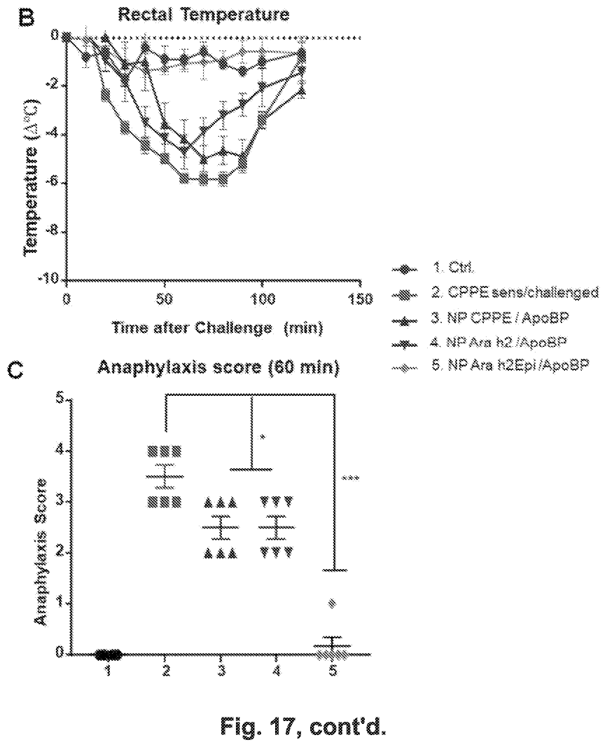

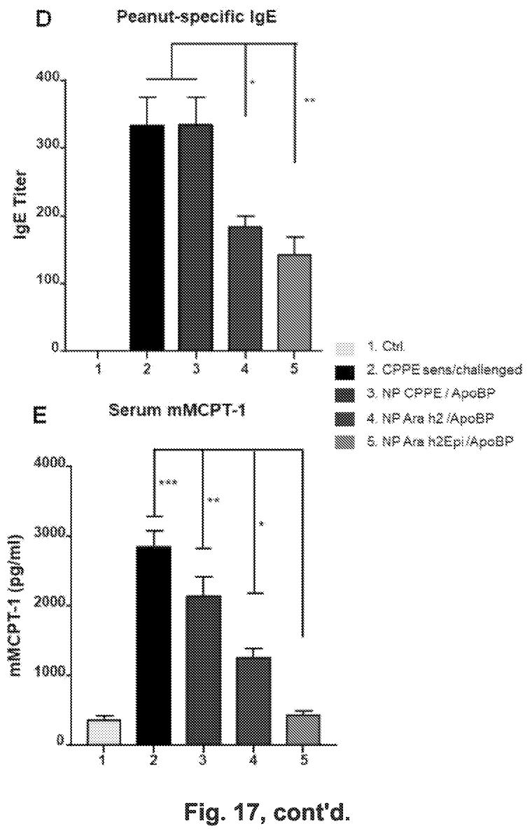

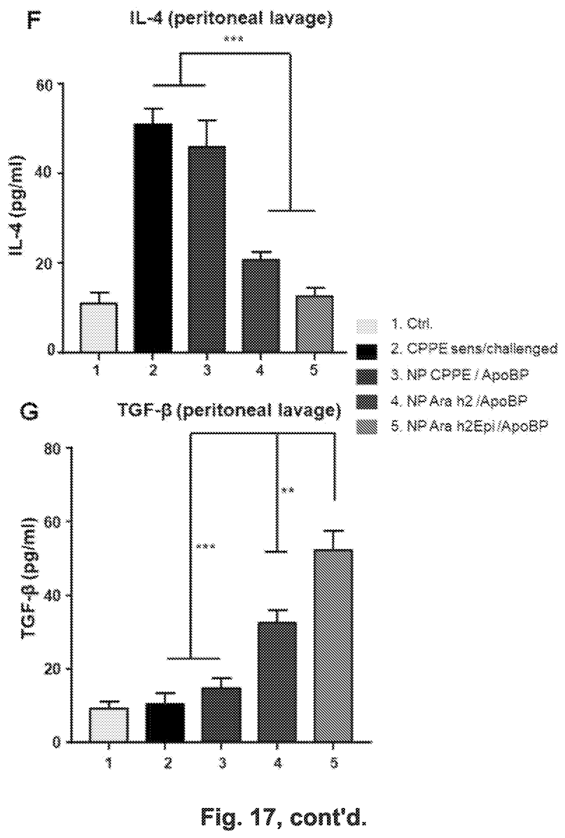

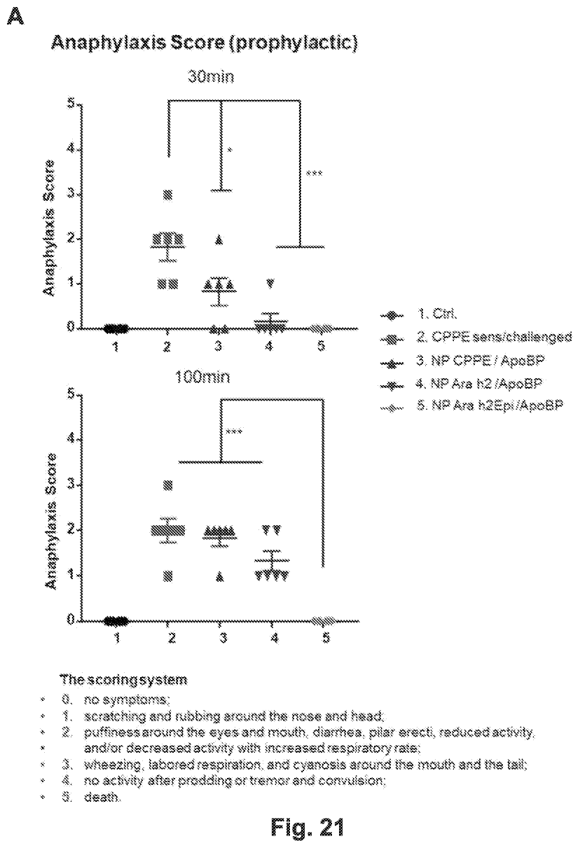

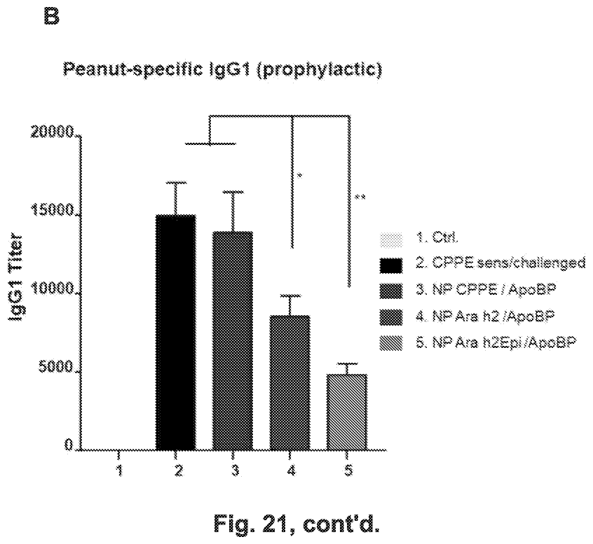

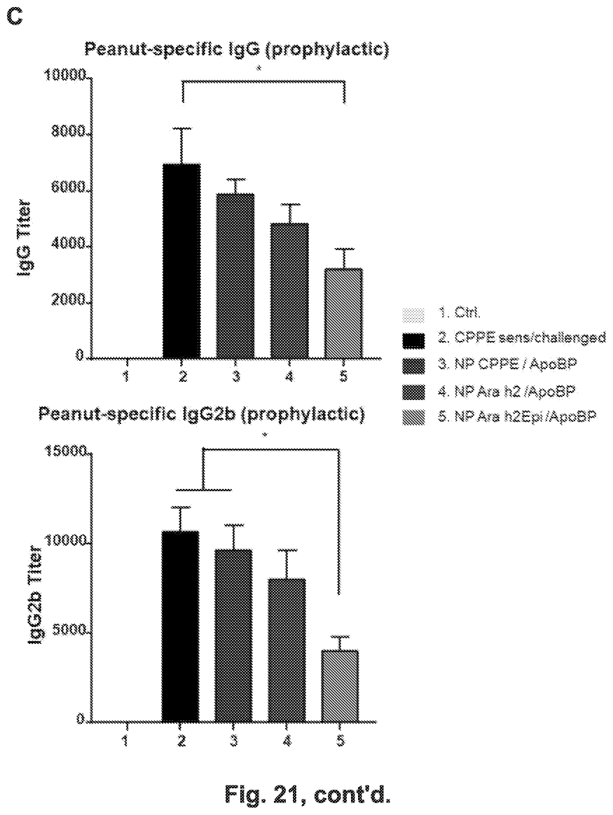

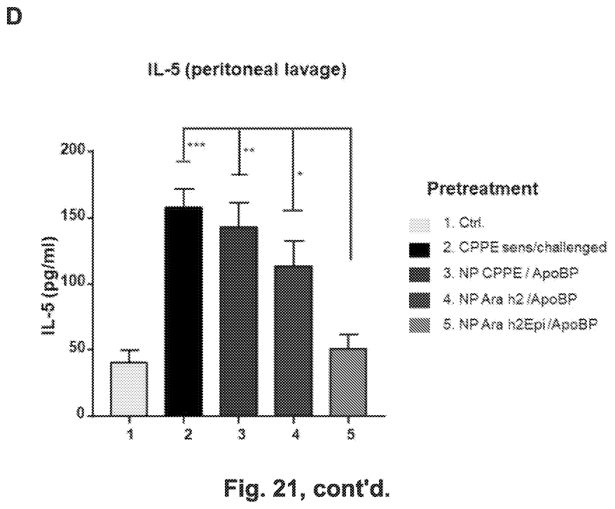

In the prophylaxis treatment experiment, animals were divided into 5 groups (n=6), which included three groups of sensitized animals, prior treated with particles containing CPPE, Ara h2, and Arah2 epitope (non-sensitized and sensitized animals not receiving pretreatment comprised the other groups) (FIG. 17, panel A). The oral anaphylaxis model was established by oral gavage, administering 200 μL of a PBS suspension containing 2 mg CPPE and 10 μg cholera toxin, once a week for 3 weeks. This was followed by 300 μL of the suspension containing 5 mg CPPE and 10 μg cholera toxin during week 4, followed by peritoneal challenge with 200 μL PBS containing 200 μg CPPE, on week 6. Prophylactic treatment was administered intravenously (IV) by injecting 500 μg of a particle suspension containing 25 μg, 4 μg and 4 μg of CPPE, Ara h2, and Ara h2 epitope, respectively, twice, seven days apart (FIG. 17, panel A). Anaphylaxis was assessed by recording core temperatures with a rectal probe (Kent Scientific) as well as visual scoring (by three independent observers) of physical manifestations of anaphylaxis, using a a 0-5 point grading scale, shown in FIG. 21, panel A (Li et al. (2000) supra; Srivastava et al. supra). In addition, blood was collected for measurement of peanut-specific IgE, IgG1, IgG, and IgG2b serum antibody titers, using an ELISA procedure, previously reported (Liu et al. (2021) supra; Srivastava et al. (2016) supra). We also assessed mouse Mast Cell Protease-1 (mMCPT-1) levels by an ELISA procedure, using the manufacturer's instructions (Thermo Fisher). Peritoneal lavage fluid was collected after 48 hours for assessment of IL-4, IL-5 and TGF-β levels by an ELISA (R&D) procedure.

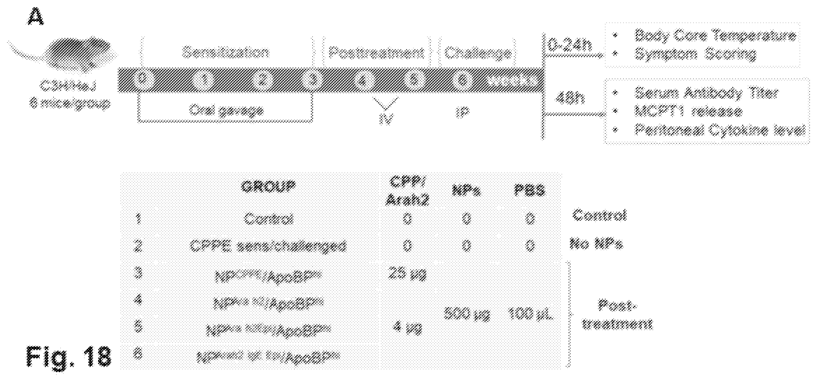

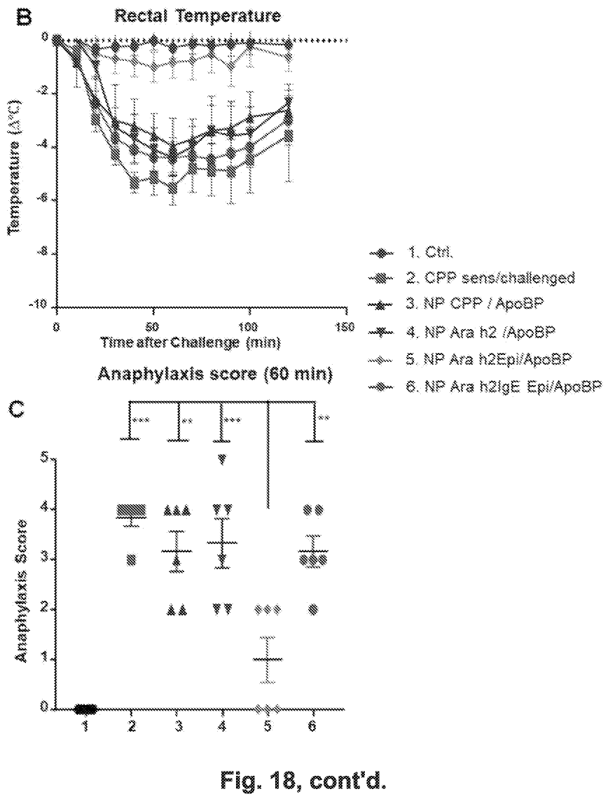

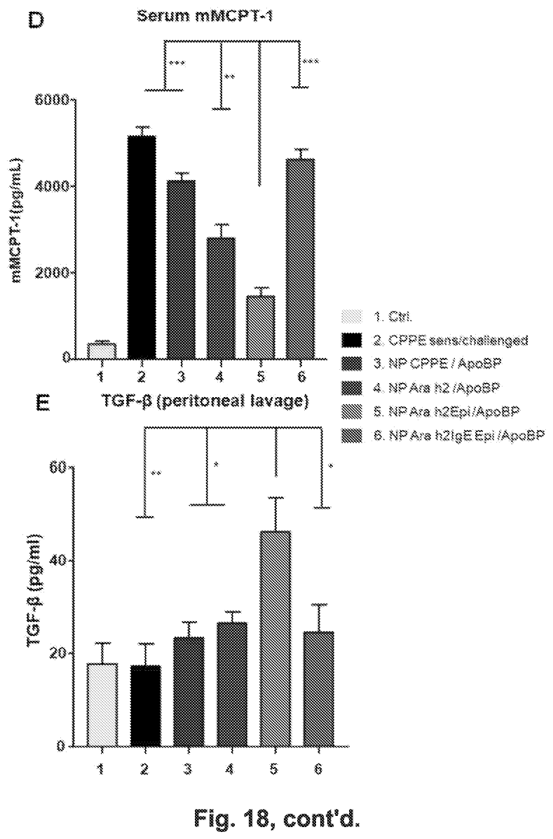

Therapeutic intervention in already-sensitized animals (n=6) followed the same procedure, except that tail vein injection of 500 μg nanoparticles, containing 25 μg, 4 μg, 4 μg, and CPPE, Ara h2, Arah2 epitope, and Arah2 IgE epitope, respectively, were administered during weeks 4 and 5, prior to peritoneal challenge (FIG. 18, panel A). Anaphylaxis monitoring and endpoint analysis proceeded as above.

To assess the durability of the tolerogenic response, mice (n=6) were prophylactically injected with 500 μg PLGA particles containing 4 μg of Ara h2 epitope (NP^{Arah2epitope/ApoBP}) on two occasions during weeks 1, 2, and 4, in advance of commencing sensitization. The mice were subsequently challenged and assessed as described above.

Assessment of CD4⁺CD25⁺FoxP3⁺Regulatory T-cell generation

Mice (n=4) received one-time IV administration of 500 μg of NP^{Arah2epitope}/ApoBP and NP^{Arah2}/ApoBP and were sacrificed 7 days later to collect splenocytes. For ELISPOT assays, splenocytes were sorted using the CD4⁺CD25⁺ Regulatory T Cell Isolation Kit (Miltenyi Biotec, Bergisch Gladbach, Germany). The sorted cells were seeded onto plates for the performance of ELISPOT assays, as per the manufacturer's guidelines (R&D). The sorted cells were also used to assess the percentage of CD4⁺CD25⁺FoxP3⁺ T-cells by flow cytometry, using cellular staining with anti-FoxP3-PE, anti-CD25-APC, and anti-CD4-AlexaFluor488 antibodies.

### Statistical Analysis

Statistical analysis was performed by one-way ANOVA or the Student t test on GraphPad Prism 7 software (GraphPad Software, La Jolla, CA) to determine the statistical differences between different groups. The results were expressed as mean±SEM and statistical significance thresholds were set at *p<0.05; **p<0.01; ***p<0.001.

### Results

### Synthesis and Characterization of Tolerogenic PLGA Nanoparticles

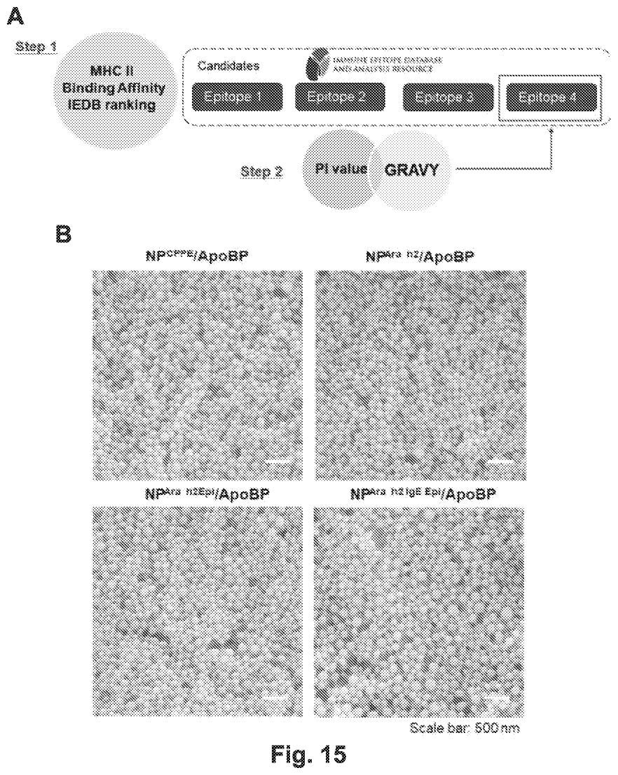

Use of the IEDB resource to identify 15-mer peptides with the highest binding affinity (IC50 values <50 nM) for murine MHC-II alleles (H2-IAb, H2-IEd and H2-IAd), yielded 4 Ara h2 sequences that were predicted as possible T-cell epitopes (FIG. 15, panel A).

TABLE 66
Illustrative Ara h2 and Ara h6 sequences predicted
as possible T-cell epitopes.

| T-cell | | | | | |
|---|---|---|---|---|---|
| Epitope | Ara | | | | Loading |
| Region | Region | Sequence | Pi | GRAVY | Capacity |
| #1 | Ara h2 | LALFLLAAHASARQQ | 11.1 | 0.767 | 1.62 + 2.11 |
| | 10-24 | (SEQ ID NO: 1198) | | | |
| #2 | Ara h2 | LRNLPQQCGLRAPQR | 12.2 | −1.027 | 3.22 + 1.96 |
| | 145-159 | (SEQ ID NO: 1199) | | | |
| #3 | Ara h2 | MAKLTILVALALFLL | 10.1 | 2.467 | 1.31 + 1.72 |
| | 1-15 | (SEQ ID NO: 1200) | | | |
| #4 | Ara h2 | SYGRDPYSPSQDPYS | 3.9 | −1.820 | 8.68 + 2.04 |
| | 59-73 | (SEQ ID NO: 1201) | | | |
| IgE | Ara h2 | HASARQQWEL | 7.8 | −1.250 | 4.75 + 0.98 |
| Epitope | 15-24 | (SEQ ID NO: 1202) | | | |
| 6 | Ara h6 | QDRQMVQQFKRELMN | | | |
| | 109-123 | (SEQ ID NO: 1203) | | | |
| 7 | Ara h6 | SAMRRERGRQGDSSS | | | |
| | 20-34 | (SEQ ID NO: 1204) | | | |
| 8 | Ara h6 | QYDSYDIRSTRSSDQ | | | |
| | 61-75 | (SEQ ID NO: 1205) | | | |

We also identified a 10-mer peptide with predicted IgE binding capacity (FIG. 15, panel A). Additional GRAVY analysis and establishment of peptide p1 were used to evaluate encapsulation efficacy in PLGA nanoparticles, based on peptide solubility and charge. This demonstrated the best cargo loading predictions for the peptide sequences that make up epitope #4 (SYGRDPYSPSQDPYS, SEQ ID NO:1201) as well as the IgE-binding sequence (HASARQQWEL (SEQ ID NO:1202)). Epitope #4 overlaps with a peptide sequence (DSYERDPYSPSQDPYSPSPY (SEQ ID NO:1206)), predicted by Prickett et al. to bind to the human HLA-DRB3*01:01 allele (Prickett et al. (2011) supra). Epitope #4 and the IgE binding peptide were selected for encapsulation into nanoparticles, in addition to CPPE and purified Ara h2, making use of our double emulsion synthesis approach (FIG. 20, panel B). These particles were subsequently used for covalent surface attachment of an ApoB-100 peptide sequence for LSEC targeting, as previously described by us (Liu et al. (2019) supra). This yielded nanoparticle batches designated as: NP^{CPP}/ApoBP, NP^{Ara h2}/ApoBP, NP^{Ara h2Epi}/ApoBP and NP^{Ara h2 IgE Epi}/ApoBP (FIG. 15, panel B). Table 67 also displays particle physicochemical characteristics including their size, size distribution, zeta potential, ligand and cargo peptide contents (BCA assay).

TABLE 67
Illustrates displays particle physicochemical characteristics including nanoparticle
size, size distribution, zeta potential, ligand and cargo peptide contents.

| | | | | | Allergen | Ligand | |
|---|---|---|---|---|---|---|---|
| | | | | Zeta | content | content | |
| | | | | potential | (μg/mg | (μg/mg | mol % |
| | Group | Size | PDI | (mV) | NP) | NP) | PLGA |
| 1 | NP^{CPPE}/ApoBP | 210.5 ± | 0.063 | −38.30 ± | 52.48 ± | 11.45 ± | 5.1 |
| | | 3.12 | | 0.11 | 1.39 | 0.32 | |
| 2 | NP^{Ara h2}/ApoBP | 207.3 ± | 0.053 | −38.16 ± | 9.31 ± | 11.82 ± | 5.1 |
| | | 1.81 | | 0.23 | 2.66 | 0.53 | |
| 3 | NP^{Ara h2Epi}/ApoBP | 205.9 ± | 0.081 | −33.28 ± | 8.68 ± | 11.55 ± | 5.1 |
| | | 2.67 | | 0.49 | 2.04 | 0.46 | |
| 4 | NP^{Ara h2 IgE Epi}/ApoBP | 202.8 ± | 0.098 | −30.39 ± | 4.75 ± | 11.32 ± | 5.1 |
| | | 5.06 | | 0.52 | 0.98 | 0.79 | |

FIG. 15, panel B demonstrates the uniformity of particle morphology and size by SEM characterization. These physicochemical characteristics are important for particle targeting and uptake by LSECs, as previously described by us (Liu et al. (2019) supra).

ELISPOT assays and flow cytometry demonstrate CD4⁺CD25⁺Foxp3⁺ Treg generation

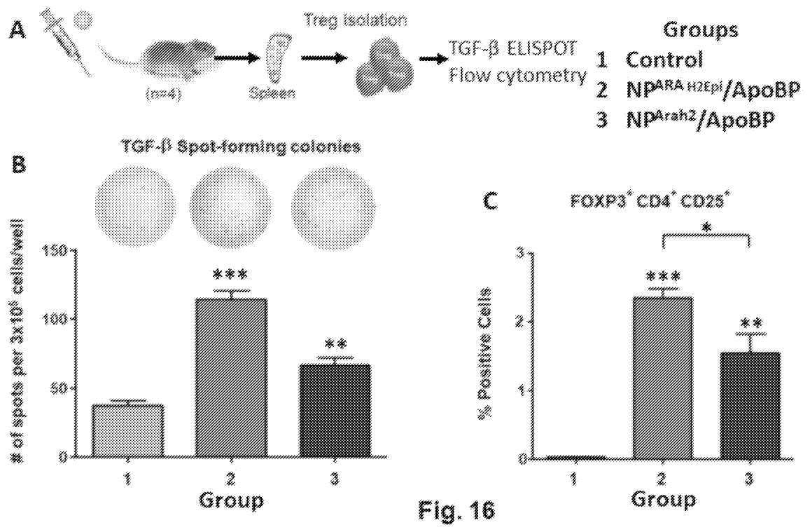

Our previous studies looking at OVA-induced allergic inflammation and anaphylaxis demonstrated that tolerogenic nanoparticles delivering a T-cell epitope was as effective or more potent than the intact allergen in promoting Treg recruitment and TGF-β release in lung and peritoneal lavage fluids (Liu et al. (2019) supra; Liu et al. (2021) supra). In order to determine whether targeted nanoparticles delivering purified Ara h2 or epitope #4 can generate Tregs in C3H/HeJ mice, CD4⁺CD25⁺ T-cells were isolated from the spleen to assess the number of TGF-β spot-forming colonies on an ELISPOT plate (FIG. 16, panel A). This demonstrated a significant increase in the number of TGF-β spot-forming colonies in response to the Ara h2 T-cell epitope (p<0.001) as well as purified Ara h2 (p<0.01) (FIG. 16, panel B). This result was also in agreement with the percentage of Foxp3⁺ cells in the selected CD4⁺CD25⁺ T-subpopulation, using a flow cytometry procedure to show significant (p<0.001 and p<0.01) results for for NP^{Arah2epitope}/ApoBP and NP^{Arah2}/ApoBP, respectively (FIG. 16, panel C).

### Prophylactic Effect of Tolerogenic Nanoparticles on Peanut-Induced Anaphylaxis

An oral anaphylaxis model was established, using CPPE sensitization and boosting, as shown in FIG. 17, panel A (Orgel & Kulis (2018) supra). Animals received IV administration of NPs incorporating CPPE, Ara h2 and Ara h2 epitope #4 on 2 occasions, 7 days apart, prior to the onset of sensitization. Following intraperitoneal challenge (week 6), sensitized animals receiving no pre-treatment, experienced a 6° C. drop in core body temperature after 60 minutes (FIG. 17, panel B). In contrast, no hypothermia was seen the non-sensitized (control) group or animals prior treated with NPs delivering the Ara h2 epitope. While there was a lesser (p<0.01) hypothermic response to purified Ara h2 prophylaxis, encapsulated CPPE had no protective effect. Monitoring of the physical manifestations of anaphylaxis further demonstrated a significant improvement (p<0.001) in the anaphylaxis score by prior treatment with the Ara h2 epitope compared to sensitized animals receiving no pretreatment (FIG. 17, panel C). Only one animal in the former group demonstrated rubbing of the nose and head at the time of maximum hypothermia (60 min), compared to the latter group, exhibiting lack of activity after prodding, wheezing and labored respiration (median score of 3.5). In contrast, animals prior treated with CPPE and Ara h2 NPs showed lesser but significant (p<0.05) improvement in anaphylaxis scores (medium scores of 2-3) (FIG. 17, panel C). These treatment responses also agreed with the anaphylaxis scores at 30 and 100 min (FIG. 15, panel A).

In addition to the impact on physical disease manifestations, pretreatment with NP^{Ara h2/ApoBP} and NP^{Ara h2Epi/ApoBP} significantly reduced (p<0.01, p<0.05), the respective peanut-specific IgE levels (FIG. 17, panel D). In addition, NP^{Ara h2Epi/ApoBP} administration also decreased peanut-specific IgG1, IgG, and IgG2b, antibody titers (FIG. 21, panels B-C). Assessment of mast cell protease (mMCPT-1) release to the serum also showed that NP^{Ara h2Epi/ApoBP} pretreatment could prevent mast cell degranulation in mice experiencing an anaphylactic response (p<0.001), including lesser effectiveness by encapsulated Ara h2 and CPPE (FIG. 17, panel E). Two days after the anaphylactic episode, the animals were sacrificed and peritoneal lavage performed to assess TH2 cytokine and TGF-β levels. Compared to the high values of IL-4 (FIG. 17, panel F) in sensitized non-treated mice, pretreatment with NP^{Ara h2/ApoBP} and NP^{Ara h2Epi/ApoBP} showed significant reductions (p<0.001) in cytokine levels (FIG. 17, panel F). The same was true for IL-5, except that the response was not as dramatic for Ara h2 (FIG. 21, panel D). Pertaining to TGF-β, pretreatment with NP^{Ara h2Epi/ApoBP} had a highly significant effect (p<0.001) on peritoneal fluid levels, which was slightly less impactful (p<0.01) than during NP^{Ara h2/ApoBP} treatment (FIG. 17, panel G). These results are in agreement with the impact of these nanoparticles on Treg generation in ELISPOT assays (FIG. 16, panels B-C).

### Post-Sensitization Impact of the Tolerogenic Nanoparticles on Peanut-Induced Anaphylaxis

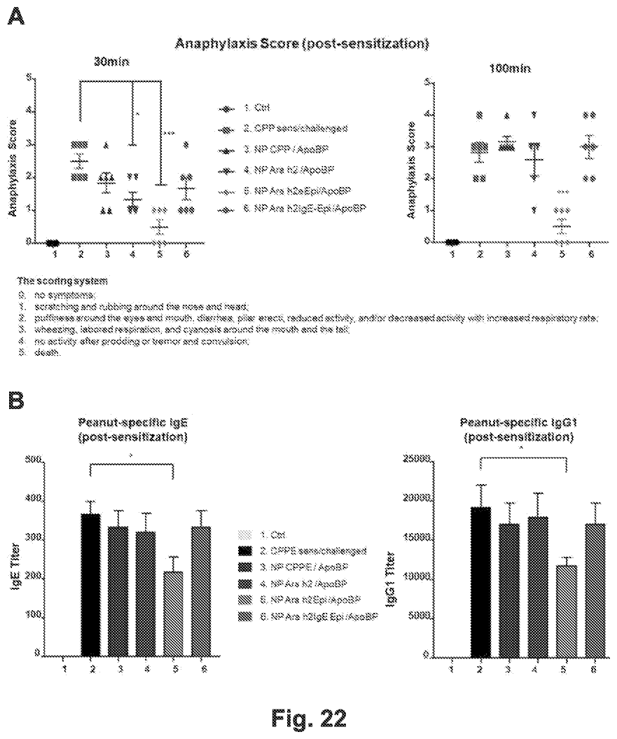

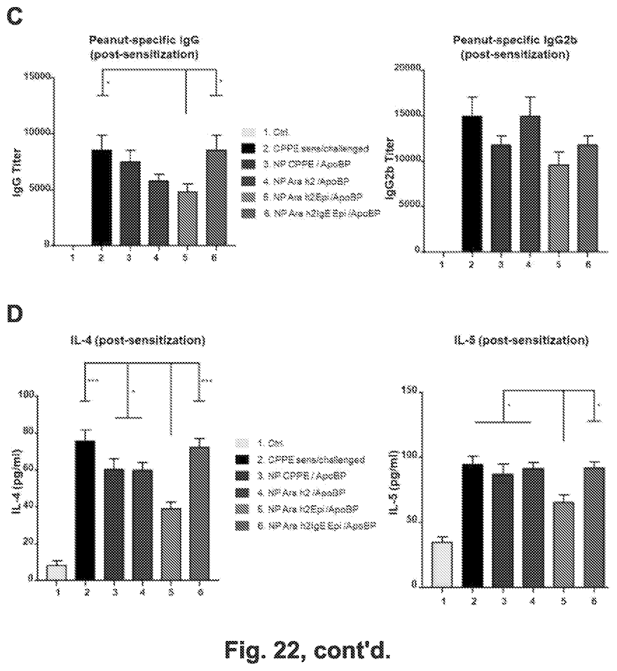

In this experiment, particle treatment commenced after sensitization to CPPE (FIG. 18, panel A). The study included the addition of a new group, namely administration of nanoparticles encapsulating a predicted IgE binding epitope, to rule out the possibility of an anaphylactic particle response, in addition to serving as a control for the T-cell epitope. Following CPPE challenge, the same anaphylactic and biomarker manifestations were assessed as described above. This demonstrated that post-sensitization treatment with NP^{Ara h2Epi/ApoBP} had comparable but lesser efficacy than prophylactic administration, while other treatments were much less effective. Noteworthy, there was no adverse response to the IgE binding peptide, which failed to exert a tolerogenic effect. Core temperature recording demonstrated that while NP^{Ara h2Epi/ApoBP} treatment effectively prevented hypothermia, other treatments had no preventative effect (FIG. 18, panel B). This was also reflected in anaphylaxis scores at the time of maximum hypothermia (60 min), with 3 NP^{Ara h2Epi/ApoBP} treated animals exhibiting facial puffiness and reduced activity (level 2), while the rest had no anaphylactic manifestations (level 0) (FIG. 18, panel C). Other treatments solicited level 3 responses, compared to level 4 in non-treated animals (FIG. 18, panel C). Comparable anaphylaxis scores were recorded at 30 and 100 mins (FIG. 22, panel A).

In agreement with the anaphylactic manifestations, mice treated with NP^{Ara h2Epi/ApoBP} showed a significant reduction in mMCPT-1 release (p<0.001), while NP^{CPPE/ApoBP} (p<0.05) and NP^{Ara h2/ApoBP}, (p<0.01) also exerted impactful responses (FIG. 18, panel D). In addition, TGF-β levels were significantly (p<0.01) increased by NP^{Ara h2Epi/ApoBP} compared to other treatment groups (FIG. 18, panel E). A less pronounced (compared to prophylactic treatment) but still significant decline in peanut-specific IgE, IgG and IgG1 antibody titers and TH2 cytokine (IL-4, IL-5) levels were observed in response to post-sensitization NP^{Ara h2Epi/ApoBP} administration (FIG. 22, panels B-D).

### Durable Tolerogenic Effect of Prophylactically Administered Nanoparticles

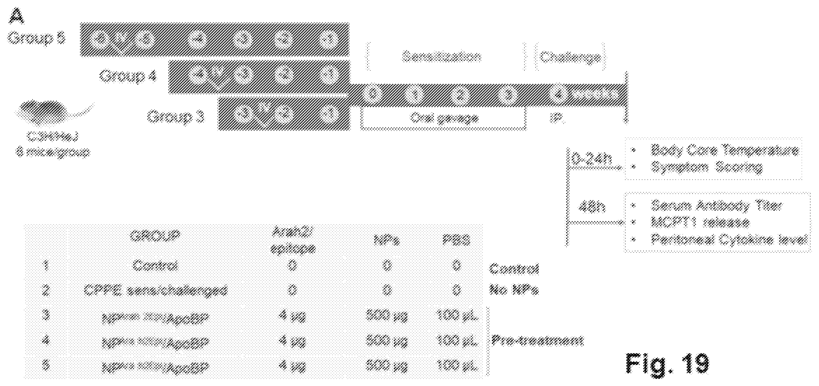

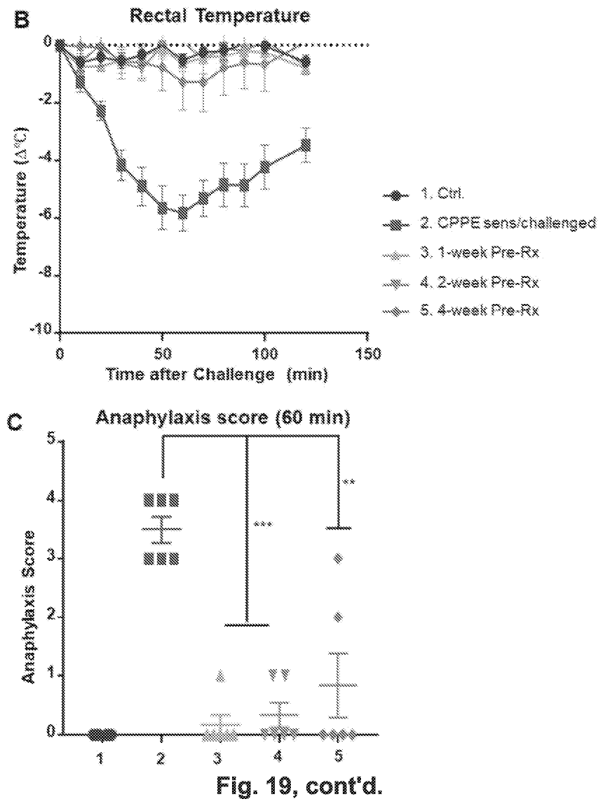

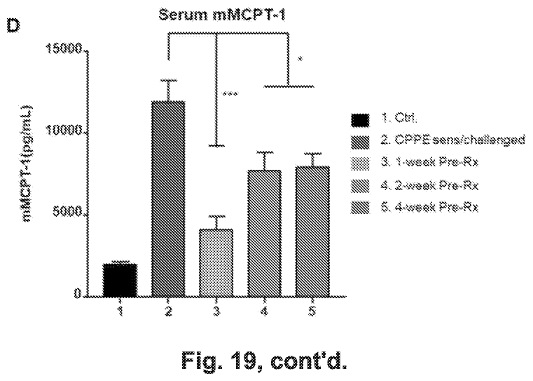

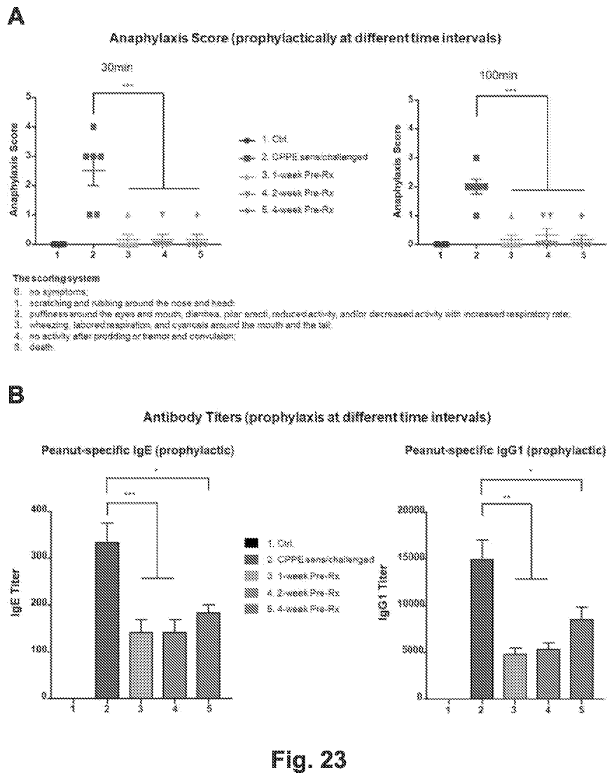

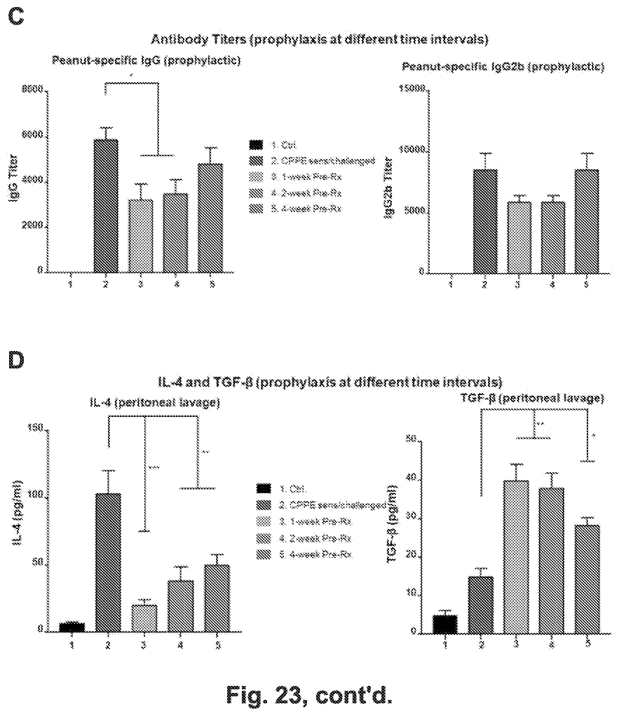

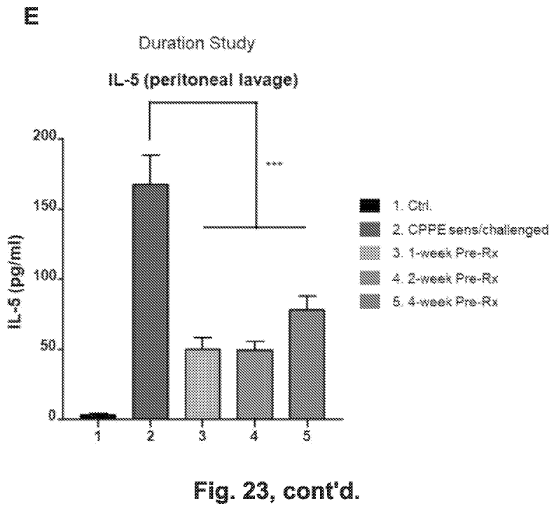

Durable immune tolerance is the ultimate goal for food allergy immunotherapy. In order to explore the duration of the prophylactic response to NP^{Ara h2Epi/ApoBP}, the nanocarrier was injected twice during weeks 1, 2 and 4, prior to commencing CPPE sensitization and anaphylaxis challenge (FIG. 19, panel A). The results demonstrated protection against the development of hypothermia and anaphylactic manifestations in all the pretreated groups, with marginally higher anaphylactic scores in animals receiving pre-treatment 4 weeks prior before commencement of sensitization (FIG. 19, panels B and C). Two out of 6 mice in the latter treatment group showed a mild reduction in activity, with wheezing and labored respiration, compared to zero or minor manifestations in other pre-treatment groups. This was comparable to anaphylactic scores at 30 and 100 mins (FIG. 23, panel A). All treatment groups showed reduced mMCPT-1 release to the serum, with animals receiving particle administration 1 week prior to commencement of sensitization, showing most prominent decline (FIG. 19, panel D). Similar results were also obtained in the assessment of anti-peanut antibody titers, TH2 cytokine and TGF-β production (FIG. 23, panels B-E). Thus, although there is some tendency for response decline with lengthening of the pre-treatment interval, there is a statistically significant suppression of anaphylaxis for at least two months between the time of initial nanoparticle administration and allergen challenge.

### DISCUSSION

In this example we demonstrate that encapsulated delivery of an Ara h2 T-cell epitope to the liver is capable of triggering a linked tolerogenic response, leading to successful suppression of the anaphylactic response to CPPE in an oral challenge model. Moreover, we demonstrate that the tolerogenic nanoparticle platform is capable of generating Tregs and TGF-β production, with a significant impact on allergic inflammation and mast cell release. The epitope-induced response was also more robust than encapsulated delivery of purified Ara h2, an IgE binding peptide and CPPE. Prophylactic administration protected against anaphylaxis for at least two months.

While a number of NP platforms, including PLGA nanoparticles, have emerged for therapeutic intervention in autoimmune and allergic disorders (Kishimoto & Maldonado (2018) supra), our approach of targeted delivery of the antigenic cargo to LSECs is unique (Liu et al. (2019) supra; Liu et al. (2021) supra). Major LSEC functions include the removal of macromolecules and particulate antigens from the blood, in addition to the ability to also act as tolerogenic APC, capable of generating CD25⁺Foxp3⁺ Tregs (Knolle & Wohlleber (2016) supra; Carambia et al. (2014) supra). While clearance of waste products from the blood has traditionally been attributed to the phagocytic capabilities of Kupffer cells (Nguyen-Lefebvre & Horuzsko (2015) supra), it is now understood that LSECs also exhibit particle uptake through clathrin-mediated endocytosis that involve stabilin, mannose and scavenger receptors (Pandey et al. (2020) supra; Sørensen et al. supra). Although it was shown that non-targeted PLGA nanoparticles in the size range 200-300 nm can be taken up by both Kupffer and LSECs (Sørensen et al. (2012) supra; Park et al. (2016) supra), we have demonstrated that covalent attachment of an Apo-BP ligand allows proportionately more carrier delivery to LSECs as a result of their preferential expression of stabilin-1 receptors (Liu et al. (2019) supra). The natural tolerogenic effect of these cells, including their ability to bind exogenous TGF-β, allows more robust Foxp3⁺ Treg generation in response to antigenic cargo (Carambia et al. (2014) supra). This agrees with the data demonstrating the generation of Ara h2-specific Tregs and TGF-β production (FIG. 16). While also effective in generating tolerogenic effects, the full-length Ara h 2 protein is not quite as effective as the epitope, which may be a reflection of improved MHC-II binding and uptake of the peptide, which is expressed in 10-fold molar access than the content of the same sequence in the intact protein.

IEDB-based predictions to identify potentially useful peptide sequences for therapeutic intervention in peanut allergy in humans have been published for dominant peanut allergens, including Ara h1 and h2 (Prickett et al. (2011) supra; Prickett et al. (2013) Clin. Exp. Allergy, 43 (6): 684-697; Pascal et al. (2013) Clin. Exp. Allergy, 43 (1): 116-127; Ramesh et al. (2016) J. Allergy Clin. Immunol. 137 (6): 1764-1771.e4; Glaspole et al. (2005) Allergy, 60 (1): 35-40). This includes prediction making of lead peptide sequences with high binding affinity for HLA-DR, -DP and -DQ to achieve widespread population coverage, as well as avoiding IgE binding sequences that could negatively impact therapeutic safety (Prickett et al. (2011) supra; Prickett et al. (2013) supra; Pascal et al. (2013) supra; Deak et al. (2017) Sci. Rep. 7 (1): 3981). The initial proof-of-principle of the translational use potential of allergen T-cell epitopes was further demonstrated by immunotherapy experiments in animals sensitized to house dust mite and cat allergens (44. O'Hehir et al. (2016) Curr. Allergy Asthma Rep. 16 (2): 14; Dorofeeva et al. (2021) Allergy, 76 (1): 131-149). Noteworthy, these studies demonstrated that animal vaccination with a single dominant T-cell epitope can introduce specific non-responsiveness to repetitive intradermal injections of the same peptide, as well as the complete allergen extract (Prickett et al. (2015) supra; O'Hehir et al. (2016) supra). The robustness of the T-cell epitope approach has also been validated in other allergen models, evolving to the use of short peptide mixtures for intradermal administration (O'Hehir et al. (2016) supra; Worm et al. (2011) J. Allergy Clin. Immunol. 127 (1): 89-97, 97.el-14). Currently, a phase 1 clinical trial is ongoing in Australia to assess the safety and tolerability of PVX108 (Aravax), an intradermal vaccine delivering a mixture of Ara h 1 and h2 epitope sequences (Australian New Zealand Clinical Trials Registry ACTRN 12617000692336) (Prickett et al. (2019) J. Allergy Clin. Immunol. 143 (2): AB431). While the efficacy and durability of this vaccine still awaits assessment, there has been a tendency in previous clinical trials for the allergen immunotherapy response to decline after therapy discontinuation, with a tendency to become resensitized to the same allergen (Sampath & Nadeau (2019) J. Clin. Invest. 129 (4): 1431-1440). It is also necessary for peanut oral immunotherapy (e.g., Palorzia), to maintain daily peanut allergen ingestion to maintain the desensitized state (Dougherty et al. (2021) supra; Santos et al. (2020) supra).

We propose that the use of a liver-targeting platform could provide a more durable and robust state of non-responsiveness by engaging a natural tolerogenic environment for Treg regeneration. While our results show that it is possible to prevent anaphylaxis for up to two months, additional research is required to address questions about response duration in the post-sensitization phase, the number of doses to administer and dosing frequency to maintain a tolerogenic response. Our platform is quite adaptable, with the possibility to improve response duration, if needed, by co-delivery of pharmaceutical agents that augment Treg stability/potency, as well as changing the polymer composition for slowing cargo release for weeks to months (Benhabbour et al. (2019) Nat. Commun. 10 (1): 4324; Swider et al. (2018) Acta Biomater. 73:38-51).

### REFERENCES

1. Finkelman (2010) Curr. Opin. Immunol. 22 (6): 783-788.
2. Wang & Sampson (2007) Clin. Exp. Allergy, 37 (5): 651-660.
3. Sicherer & Sampson (2018) J. Allergy Clin. Immunol. 141 (1): 41-58.
4. Branum & Lukacs (2009) Pediatrics, 124 (6): 1549-1555.
5. Bock et al. (2001) J. Allergy Clin. Immunol. 107 (1): 191-193.
6. Boyce et al. (2010) J. Allergy Clin. Immunol. 126 (6): 1105-1118.
7. Varshney et al. (2011) J. Allergy Clin. Immunol. 127 (3): 654-660.
8. Chin et al. (2013) J. Allergy Clin. Immunol. 132 (2): 476-478.e2.
9. Nurmatov et al. (2012) Cochrane Database Syst. Rev. (9).
10. Sheikh & Burks (2013) Expert Rev. Clin. Immunol. 9 (6): 551-560.
11. Dougherty et al. (2021) Ann. Pharmacother. 55 (3): 344-353.
12. Vickery et al. (2018) N. Engl. J. Med. 379 (21): 1991-2001.
13. Santos et al. (2020) J. Allergy Clin. Immunol. 145 (1): 440-443.e5.
14. Mullard (2020) Nat Rev Drug Discov. 19 (3): 156.
15. Patrawala et al. (2020) Curr. Allergy Asthma Rep. 20 (5): 14.
16. Wasserman et al. (2021) J. Allergy Clin. Immunol: In Practice, 9 (5): 1826-1838.e8.
17. Akdis (2012) Nat. Med. 18 (5): 736-749.
18. Johnson-Weaver et al. (2018) Front. Immunol. 9:2156.
19. Liu et al. (2019) ACS Nano, 13 (4): 4778-4794.
20. Liu et al. (2021) ACS Nano, 15 (1): 1608-1626.
21. Tiegs & Lohse (2010) J. Autoimmun. 34 (1): 1-6.
22. Vickery et al. (2011) J. Allergy & Clin. Immunol. 127 (3): 576-586.
23. Knolle & Wohlleber (2016) Cell. Mol. Immunol. 13 (3): 347-353.
24. Crispe, (2011) J. Hepatol. 54 (2): 357-365.
25. Horst et al. (2016) Cell. Mol. Immunol. 13 (3): 277-292.
26. Carambia et al. (2014) J. Hepatol. 61 (3): 594-9.
27. Carambia et al. (2015) J. Hepatol. 62 (6): 1349-1356.
28. Hemmings et al. (2020) J. Allergy Clin. Immunol. 146 (3): 621-630.e5.
29. Li et al. (2000) J. Allergy Clin. Immunol. 106 (1, Part 1): 150-158.
30. Srivastava et al. (2016) J. Allergy Clin. Immunol. 138 (2): 536-543.e4.
31. Prickett et al. (2011) J. Allergy Clin. Immunol. 127 (3): 608-615.e5.
32. Orgel & Kulis (2018) A Mouse Model of Peanut Allergy Induced by Sensitization Through the Gastrointestinal Tract. In Type 2 Immunity: Methods and Protocols, Reinhardt, R. L., Ed. Springer New York: New York, NY, pp 39-47.
33. Kishimoto & Maldonado (2018) Front. Immunol. 9:230.
34. Nguyen-Lefebvre & Horuzsko (2015) J. Enzymol. Metab. 1 (1): 101.
35. Pandey et al. (2020) Front. Physiol. 11:873-873.
36. Sørensen et al. (2012) Am. J. Physiol. Regul. Integr. Comp. Physiol. 303 (12): R1217-R1230.
37. Park et al. (2016) Nanomed. Nanotechnol. Biol. Med. 12 (5): 1365-1374.
38. Prickett et al. (2015) Clin. Exp. Allergy, 45 (6): 1015-1026.
39. Prickett et al. (2013) Clin. Exp. Allergy, 43 (6): 684-697.
40. Pascal et al. (2013) Clin. Exp. Allergy, 43 (1): 116-127.
41. Ramesh et al. (2016) J. Allergy Clin. Immunol. 137 (6): 1764-1771.e4.
42. Glaspole et al. (2005) Allergy, 60 (1): 35-40.
43. Deak et al. (2017) Sci. Rep. 7 (1): 3981.
44. O'Hehir et al. (2016) Curr. Allergy Asthma Rep. 16 (2): 14.
45. Dorofeeva et al. (2021) Allergy, 76 (1): 131-149.
46. Worm et al. (2011) J. Allergy Clin. Immunol. 127 (1): 89-97, 97.e1-14.
47. Prickett et al. (2019) J. Allergy Clin. Immunol. 143 (2): AB431.
48. Sampath & Nadeau (2019) J. Clin. Invest. 129 (4): 1431-1440.
49. Benhabbour et al. (2019) Nat. Commun. 10 (1): 4324.
50. Swider et al. (2018) Acta Biomater. 73:38-51.
51. Li et al. (2011) Am. J. Physiol. Gastrointest. Liver Physiol. 300 (1): G71-G81.
52. Shetty et al. (2018) Nat. Rev. Gastroenterol. & Hepatol. 15 (9): 555-567.
53. Thomson & Knolle (2010) Nat. Rev. Immunol. 10 (11): 753-766.
54. Horst et al. (2021) Cell. Mol. Immunol. 18 (1): 92-111.
55. Sharma et al. (2018) Front. Immunol. 9:883.
56. Kyte & Doolittle (1982) J. Mol. Biol. 157 (1): 105-132.

### Supporting Information

FIG. 20 outlines the establishment of a tolerogenic nanoparticle platform for treatment of peanut allergy and anaphylaxis. FIG. 20, panel A depicts the experimental design for using the tolerogenic nanoparticle platform to deliver antigenic cargo to LSECs in the liver. LSECs act as antigen-presenting cells for the presentation of antigenic epitopes by MHC-II to naive T-cells, which are induced to develop into allergen-specific Foxp3⁺ Tregs. The Tregs can be recruited from the liver to sites of allergic inflammation and mast cell triggering by allergen-specific IgE to initiate anaphylactic responses. FIG. 20, panel B depicts a test strategy for implementing the use of tolerogenic nanoparticles in the peanut oral allergy model. The mice are sensitized by oral gavage and challenged by intraperitoneal injection with the crude peanut protein extract. The tolerogenic nanoparticles are administered by tail vein injection, both prophylactically and therapeutically (following animal sensitization). The physical parameters of anaphylaxis that were monitored include core body temperature and observation of manifestations of anaphylaxis, as explained in FIG. 21. We also tracked biomarkers, including antibody titers to peanut allergen and release of the mast cell protease, MCPT-1, to the serum. The late-phase response is assessed by measuring TH2 cytokine release in the peritoneal fluid.

FIG. 21 shows additional data collected during the execution of the prophylactic anaphylaxis model in FIG. 17. FIG. 21, panel A provides anaphylaxis scores at 30 and 100 min following CPPE challenge, while panels B and C show peanut-specific IgG1, IgG and IgG2b antibody titers and panel D shows IL-5 level in the peritoneal lavage fluid.

FIG. 22 shows additional data collected during the execution of the post-sensitization anaphylaxis intervention in FIG. 18. FIG. 22, panel A shows anaphylaxis score at 30 and 100 min after CPPE challenge, panels B and C show peanut-specific IgE, IgG1, IgG and IgG2b antibody titers, and panel D shows IL-4 and IL-5 levels in the peritoneal lavage fluid.

FIG. 23 shows additional data collection during execution of the experiment in FIG. 6, looking at the impact of different time intervals of prophylactic treatment. FIG. 23, panel A shows anaphylaxis scores at 30 and 100 min after DPPE challenge, while panels B and C peanut-specific IgE, IgG1, IgG and IgG2b antibody titers., and panels D and E show IL-4, TGF-β, and IL-5, level in the peritoneal lavage fluid.

### Example 4

### Design of Coding Sequences for the Expression of Ara h 2 and 6 Epitopes in Preclinical Animal Studies

We have already described how use of the NIH-supported IEDB epitope database and prediction resource (IEDB.org), allowed us to perform T cell epitope selection for murine experimentation, beginning with the entry of Ara h2 and 6 protein sequences into the database, followed by searching the MHC-II binding interactions to H2-IAb, H2-IEd and H2-IAd. We selected the epitopes ranking in the top 20% in terms of MHC II binding affinity. These epitope predictions are included in Table 68.

TABLE 68
Ara-h2, Ara-h6 and their T cell epitope predictions
for synthesis of PLGA NPs

| | | | | | Loading |
|---|---|---|---|---|---|
| | | | | | Capacity |
| Epitope | Specifications | Sequence | pI | GRAVY | (μg/mg) |
| 1 | Ara h2 10-24 | LALFLLAAHASA | 11.1 | 0.767 | 1.62 + 2.11 |
| | | RQQ (SEQ ID | | | |
| | | NO: 1198) | | | |
| 2 | Ara h2 145-159 | LRNLPQQCGLRA | 12.2 | −1.027 | 3.22 + 1.96 |
| | | PQR (SEQ ID | | | |
| | | NO: 1199) | | | |
| 3 | Ara h2 1-15 | MAKLTILVALAL | 10.1 | 2.467 | 1.31 + 1.72 |
| | | FLL (SEQ ID | | | |
| | | NO: 1200) | | | |
| 4 | Ara h2 59-73 | SYGRDPYSPSQD | 3.9 | −1.820 | 8.68 + 2.04 |
| | | PYS (SEQ ID | | | |
| | | NO: 1201) | | | |
| 5 | Ara h6 109-123 | QDRQMVQQFKRE | 10.1 | −1.520 | NA |
| | | LMN (SEQ ID | | | |
| | | NO: 1203) | | | |
| 6 | Ara h6 20-34 | SAMRRERGRQGD | 11.5 | −1.920 | NA |
| | | SSS (SEQ ID | | | |
| | | NO: 1204) | | | |
| 7 | Ara h6 61-75 | QYDSYDIRSTRS | 4.2 | −1.900 | NA |
| | | SDQ (SEQ ID | | | |
| | | NO: 1205) | | | |

Similar to our approach for constructing integrated human cDNA coding sequences for multi-epitope delivery for peanut immunotherapy, we have used the same approach to design murine mRNA vaccines for the peptide sequences in Table 68.

Reverse translation of above peptide sequences yielded the following cDNA constructs:

| | Ara h2 Epitope 1: |
|---|---|
| (SEQ ID NO: 1214) | |
| | LALFLLAAHASARQQ |
| (SEQ ID NO: 1215) | |
| | ctggcgctgtttctgctggcggcgcatgcgagcgcgcgccagcag |
| | Ara h2 Epitope 2: |
| (SEQ ID NO: 1216) | |
| | LRNLPQQCGLRAPQR |
| (SEQ ID NO: 1217) | |
| | Ctgcgcaacctgccgcagcagtgcggcctgcgcgcgccgcagcgc |
| | Ara h2 Epitope 3: |
| (SEQ ID NO: 1218) | |
| | MAKLTILVALALFLL |
| (SEQ ID NO: 1219) | |
| | Atggcgaaactgaccattctggtggcgctggcgctgtttctgctg |
| | Ara h2 Epitope 4: |
| (SEQ ID NO: 1220) | |
| | SYGRDPYSPSQDPYS |
| (SEQ ID NO: 1221) | |
| | Agctatggccgcgatccgtatagcccgagccaggatccgtatagc |
| | Ara h6 Epitope 5: |
| (SEQ ID NO: 1222) | |
| | QDRQMVQQFKRELMN |
| (SEQ ID NO: 1223) | |
| | Caggatcgccagatggtgcagcagtttaaacgcgaactgatgaac |
| | Ara h6 Epitope 6: |
| (SEQ ID NO: 1224) | |
| | SAMRRERGRQGDSSS |
| (SEQ ID NO: 1225) | |
| | Agcgcgatgcgccgcgaacgcggccgccagggcgatagcagcagc |
| | Ara h6 Epitope 7: |
| (SEQ ID NO: 1226) | |
| | QYDSYDIRSTRSSDQ |
| (SEQ ID NO: 1227) | |
| | Cagtatgatagctatgatattcgcagcacccgcagcagcgatcag |

Because these epitopes are generated from a murine source, we will use a mouse transferrin receptor sequence for endosomal targeting. The amino acid sequence for this protein in Mus musculus is:

(SEQ ID NO: 1228)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDVPTSSRLYWADLKTLLSEKLNSIEFADTIKQLS
QNTYTPREAGSQKDESLAYYIENQFHEFKFSKVWRDEHYVKIQVKSSIGQ
NMVTIVQSNGNLDPVESPEGYVAFSKPTEVSGKLVHANFGTKKDFEELSY
SVNGSLVIVRAGEITFAEKVANAQSFNAIGVLIYMDKNKFPVVEADLALF
GHAHLGTGDPYTPGFPSFNHTQFPPSQSSGLPNIPVQTISRAAAEKLFGK
MEGSCPARWNIDSSCKLELSQNQNVKLIVKNVLKERRILNIFGVIKGYEE
PDRYVVVGAQRDALGAGVAAKSSVGTGLLLKLAQVFSDMISKDGFRPSRS
IIFASWTAGDFGAVGATEWLEGYLSSLHLKAFTYINLDKVVLGTSNFKVS
ASPLLYTLMGKIMQDVKHPVDGKSLYRDSNWISKVEKLSFDNAAYPFLAY
SGIPAVSFCFCEDADYPYLGTRLDTYEALTQKVPQLNQMVRTAAEVAGQL
IIKLTHDVELNLDYEMYNSKLLSFMKDLNQFKTDIRDMGLSLQWLYSARG
DYFRATSRLTTDFHNAEKTNRFVMREINDRIMKVEYHFLSPYVSPRESPF
RHIFWGSGSHTLSALVENLKLRQKNITAFNETLFRNQLALATWTIQGVAN
ALSGDIWNIDNEF
and
the corresponding TfR 1-118
amino acid sequence is:
(SEQ ID NO: 1229)
MMDQARSAFSNLFGGEPLSYTRFSLARQVDGDNSHVEMKLAADEEENADN
NMKASVRKPKRFNGRLCFAAIALVIFFLIGFMSGYLGYCKRVEQKEECVK
LAETEETDKSETMETEDV.

Reverse translation of the mouse TfR 1-118 domain into cDNA provided the sequence:

(SEQ ID NO: 1230)
atgatggatcaggcgcgcagcgcgtttagcaacctgtttggcggcgaacc
gctgagctatacccgctttagcctggcgcgccaggtggatggcgataaca
gccatgtggaaatgaaactggcggcggatgaagaagaaaacgcggataac
aacatgaaagcgagcgtgcgcaaaccgaaacgctttaacggccgcctgtg
ctttgcggcgattgcgctggtgattttttttctgattggctttatgagcg
gctatctgggctattgcaaacgcgtggaacagaaagaagaatgcgtgaaa
ctggcggaaaccgaagaaaccgataaaagcgaaaccatggaaaccgaaga
tgtg.

Design of an integrated cDNA sequence that places TfR 1-118 upstream of the nucleotide sequences for murine Ara h2 epitopes with cleavable linkages and the addition of initiation and stop codons, yields the cDNA construct in Table 69.

TABLE 69
cDNA sequence encoding the mouse Ara h2
epitopes, separated by cleavable linkers.

ATGatgatggatcaggcgcgcagcgcgtttagcaacctgtttggcggcga
accgctgagctatacccgctttagcctggcgcgccaggtggatggcgata
acagccatgtggaaatgaaactggcggcggatgaagaagaaaacgcggat
aacaacatgaaagcgagcgtgcgcaaaccgaaacgctttaacggccgcct
gtgctttgcggcgattgcgctggtgattttttttctgattggctttatga
gcggctatctgggctattgcaaacgcgtggaacagaaagaagaatgcgtg
aaactggcggaaaccgaagaaaccgataaaagcgaaaccatggaaaccga
agatgtgGgcggcggcggcagccgcgtgcgccgcggcggcggcggcagcc
tggcgctgtttctgctggcggcgcatgcgagcgcgcgccagcagGgcggc
ggcggcagccgcgtgcgccgcggcggcggcggcagcctgcgcaacctgcc
gcagcagtgcggcctgcgcgcgccgcagcgcGgcggcggcggcagccgcg
tgcgccgcggcggcggcggcagcatggcgaaactgaccattctggtggcg
ctggcgctgtttctgctgGgcggcggcggcagccgcgtgcgccgcggcgg
cggcggcagcagctatggccgcgatccgtatagcccgagccaggatccgt
atagccgcgtgcgccgcTAA (SEQ ID NO: 1231)

Design of a similar-organized integrated cDNA constructs that encodes for Ara h2 epitopes or a combination of Ara h2 and h6 epitopes are shown in Tables 70 and 71. respectively.

TABLE 70
cDNA sequence encoding the mouse Ara h2
epitopes, separated by cleavable linkers.

atgatgatggatcaggcgcgcagcgcgtttagcaacctgtttggcggcga
accgctgagctatacccgctttagcctggcgcgccaggtggatggcgata
acagccatgtggaaatgaaactggcggcggatgaagaagaaaacgcggat
aacaacatgaaagcgagcgtgcgcaaaccgaaacgctttaacggccgcct
gtgctttgcggcgattgcgctggtgattttttttctgattggctttatga
gcggctatctgggctattgcaaacgcgtggaacagaaagaagaatgcgtg
aaactggcggaaaccgaagaaaccgataaaagcgaaaccatggaaaccga
agatgtgggcggcggcggcagccgcgtgcgccgcggcggcggcggcagcc
tggcgctgtttctgctggcggcgcatgcgagcgcgcgccagcagggcggc
ggcggcagccgcgtgcgccgcggcggcggcggcagcctgcgcaacctgcc
gcagcagtgcggcctgcgcgcgccgcagcgcggcggcggcggcagccgcg
tgcgccgcggcggcggcggcagcatggcgaaactgaccattctggtggcg
ctggcgctgtttctgctgggcggcggcggcagccgcgtgcgccgcggcgg
cggcggcagcagctatggccgcgatccgtatagcccgagccaggatccgt
atagccgcgtgcgccgctaa SEQ ID NO: 1232

Table 71 shows codon optimization for the coding sequences for Ara h2 as shown in Tables 69 and 70.

TABLE 7
1Codon optimized cDNA sequences for the
constructs shown in Tables 69 and 70.
Codon optimized cDNA sequence encoding
the mouse Ara h2 epitopes.
ATGATGATGGACCAGGCTCGTTCAGCCTTTAGTAACCTTTTTGGAGGTGA
GCCGCTGAGTTACACCCGCTTCTCCTTGGCACGACAAGTCGACGGAGATA
ACAGCCATGTGGAGATGAAACTCGCTGCAGATGAAGAGGAGAATGCTGAC
AACAACATGAAGGCCTCTGTGAGGAAACCAAAAAGGTTCAATGGCCGGCT
CTGCTTTGCTGCCATAGCCCTGGTCATCTTCTTCCTGATTGGCTTTATGA
GCGGCTACCTGGGGTACTGCAAGAGAGTAGAGCAGAAGGAAGAATGTGTG
AAACTAGCAGAGACAGAGGAAACTGACAAGAGTGAAACCATGGAGACTGA
AGATGTGGGAGGAGGCGGCTCCCGTGTAAGGAGGGGCGGGGGCGGATCCC
TGGCCCTCTTTTTACTAGCGGCTCACGCCAGCGCACGCCAGCAAGGGGGT
GGCGGTTCAAGAGTTCGCAGAGGTGGAGGGGGGTCTCTTCGGAATTTGCC
ACAGCAGTGTGGCCTGAGGGCCCCCCAGAGGGGGGGAGGTGGCTCTAGAG
TCAGACGTGGTGGTGGGGGGTCCATGGCCAAGCTGACAATCCTGGTGGCT
CTTGCATTGTTCCTCCTGGGCGGAGGGGGCAGCCGCGTGCGGCGAGGCGG
TGGGGGATCATCCTATGGAAGAGATCCCTATTCTCCTAGCCAGGACCCTT
ATTCGCGAGTTCGGCGGTGA (SEQ ID NO: 1235)
Average GC content: 57.36%
Codon optimized cDNA sequence encoding
the mouse Ara h2 epitopes.
ATGATGATGGACCAGGCCCGCTCGGCCTTCAGCAACCTGTTTGGGGGAGA
GCCATTGAGTTACACTAGGTTTAGCTTGGCAAGGCAGGTGGATGGGGACA
ATTCCCATGTGGAGATGAAACTCGCCGCAGATGAAGAGGAAAATGCTGAC
AACAACATGAAGGCCTCTGTGAGGAAACCCAAAAGGTTCAATGGCAGGCT
GTGCTTTGCTGCGATTGCTCTTGTCATTTTCTTTCTCATAGGGTTCATGA
GCGGCTACCTGGGCTACTGCAAAAGAGTCGAGCAGAAGGAAGAATGTGTG
AAGCTGGCCGAGACAGAAGAAACGGACAAGAGTGAGACCATGGAGACTGA
AGATGTGGGCGGCGGAGGCTCTCGGGTCAGAAGGGGAGGGGGTGGATCCC
TTGCACTATTTTTATTAGCAGCCCACGCGTCTGCCCGGCAACAAGGGGGA
GGCGGCTCTCGAGTGAGACGAGGGGGCGGTGGCAGCCTGAGGAACCTTCC
TCAGCAGTGTGGCCTGCGCGCACCACAGCGCGGTGGCGGAGGATCGCGTG
TTCGGAGAGGAGGAGGAGGCAGCATGGCCAAGTTGACCATCCTGGTGGCT
CTAGCTCTCTTCCTGCTCGGCGGAGGGGGTTCACGGGTCCGTCGTGGCGG
GGGTGGGTCTAGCTATGGTAGAGATCCTTATAGTCCGTCTCAGGACCCCT
ACAGTGGGGGCGGAGGTTCACGTGTACGCCGAGGAGGAGGGGGGAGCCAA
GACAGACAGATGGTGCAGCAGTTCAAGCGGGAGCTGATGAATGGCGGTGG
CGGCAGCCGCGTTAGACGAGGGGGCGGGGGATCATCAGCTATGAGAAGAG
AGCGGGGTCGACAAGGAGATTCCAGCAGTGGGGGAGGGGGCTCACGGGTA
CGCCGCGGAGGTGGAGGTTCCCAGTACGACTCCTATGACATCAGATCAAC
AAGGTCCTCCGATCAGCGGGTTAGAAGGTGA (SEQ ID NO: 1236)
Average GC content: 58.82%

The various nucleic acid sequences are readily incorporated into tolerogenic nanoparticle formulations as described above.

### Example 5

Design of Nanoparticle with Improved LSEC Targeting and Treg Stimulation.

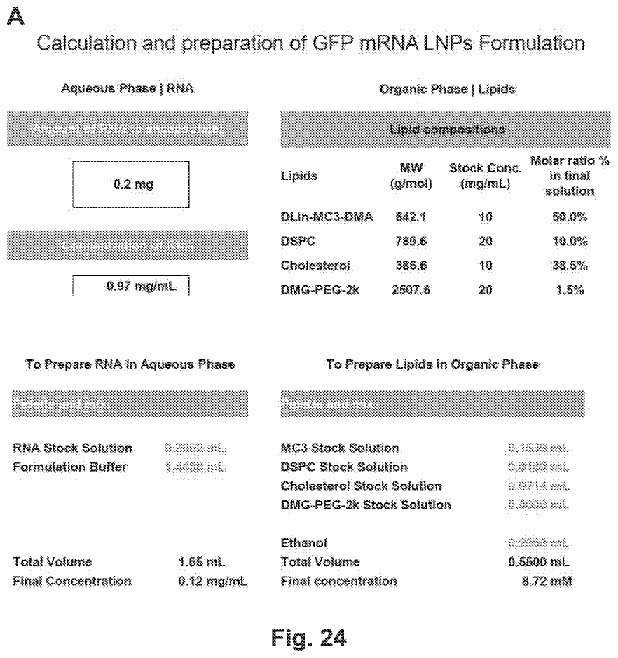

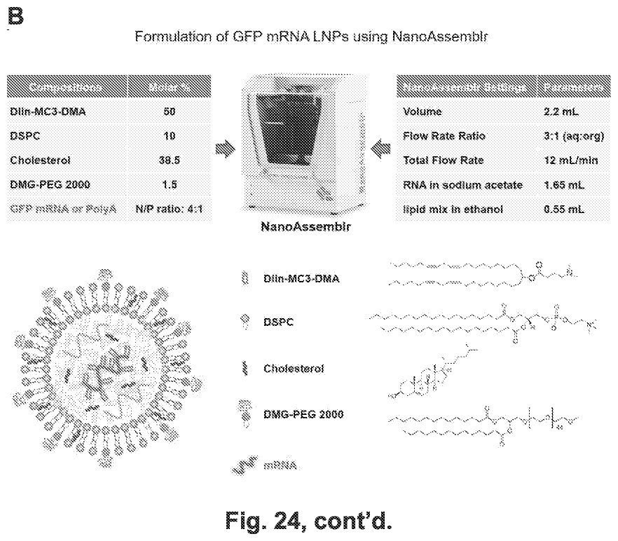

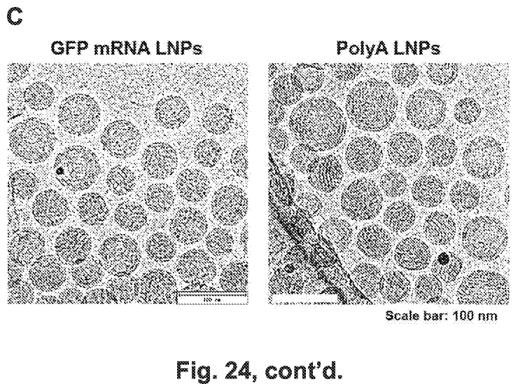

We have described the design of an mRNA-LNP platform for epitope delivery to the liver, with a view to obtaining tolerogenic therapy for allergic and autoimmune diseases (see, e.g., FIG. 24, panels A-C). As a first step, we constructed suitable nanoparticles, first used to demonstrate delivery of a GFP mRNA construct, which is compared to using a non-expressing polyA construct in LNP particle development. In order to consider a lipid composition for LNP trafficking to the liver, we drew on the composition of the FDA-approved Onpattro LNP platform. Following calculations for the composition of Aqueous and Organic Phases that can be combined by a Microfluidics blender, the NanoAssemblr Benchtop obtained from Precision NanoSystems Inc., protocols were established for synthesizing our in-house mRNA delivering LNPs. This necessitated several changes to key parameters to obtain successful synthesis, including adjustment of the Flow Rate Ratio to 3:1 (aqueous: organic), adjusting the Total Flow Rate to 12 mL/min, and keeping the N/P ratio to 4:1 (see, e.g., FIG. 24, panels A-C). Particle characterizations were carried out to determine the size, zeta potential and mRNA encapsulation efficiency (EE %) as shown in Table 72.

TABLE 72
Characterization of mRNA LNPs. The size and the surface potential
of the purified LNP samples were determined by dynamic light
scattering (DLS), using a Zetasizer Nano ZS from Malvern Instruments
Ltd. The encapsulation efficiency (EE %) of mRNA was determined
using RIBOGREEN ®, a a dye that becomes fluorescent when
bound to single-stranded mRNA, without entering the LNPs. The
encapsulation efficiency (EE %) of GFP-mRNA LNPs and Poly A LNPs
was 91.8 and 97.9%, respectively. This serves to demonstrate
that GFP mRNA or Poly A is located inside the LNPs.

| | GFP mRNA LNP | PolyA LNP |
|---|---|---|
| Size (nm) in PBS | 127.3 ± 0.9 | 110.7 ± 2.1 |
| PDI | 0.71 ± 0.004 | 0.182 ± 0.018 |
| Zeta potential (mV) | −5.54 ± 4.19 | −11.43 ± 3.94 |
| EE (%) | 91.8 ± 0.06 | 97.9 ± 0.05 |

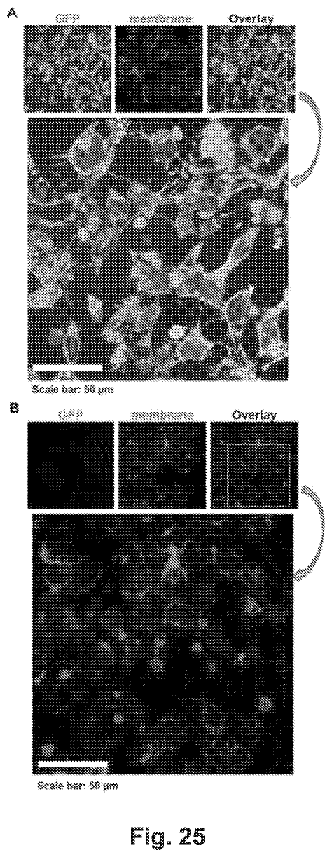

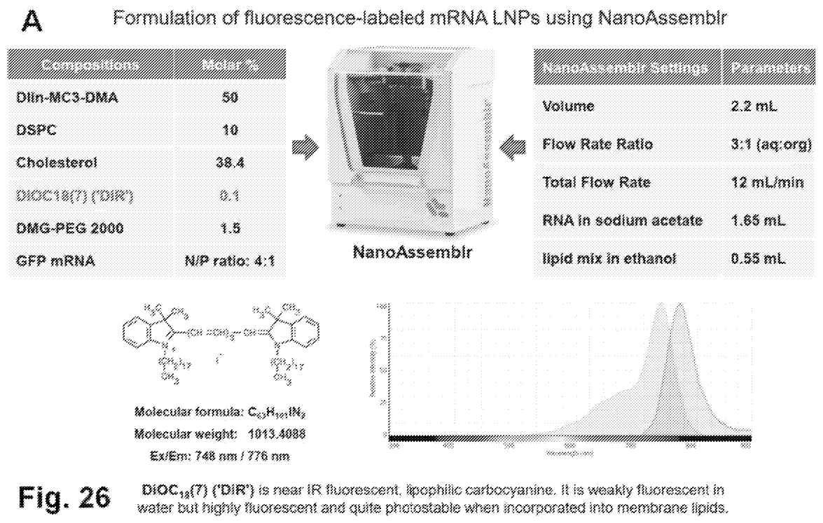

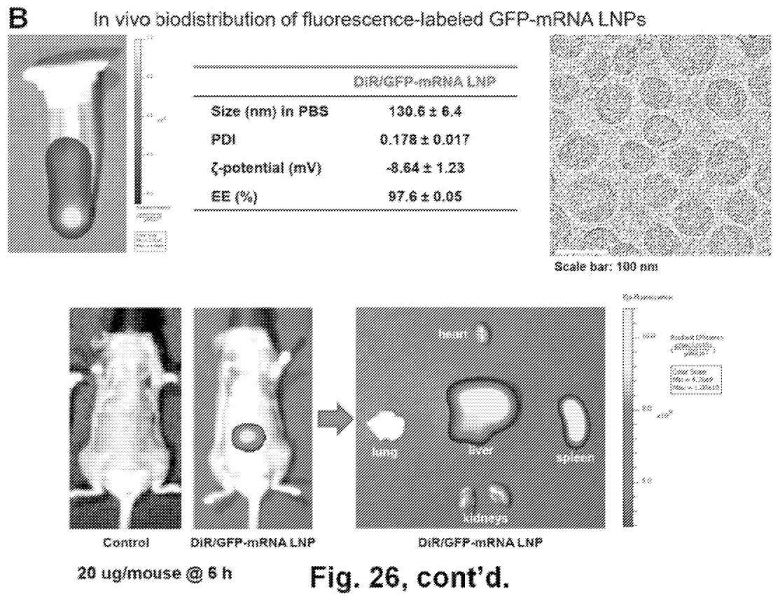

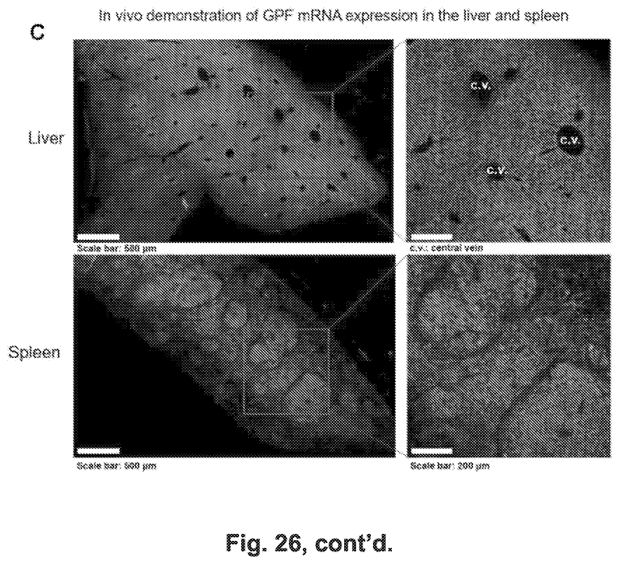

The LNP's samples were also imaged by cryogenic electron microscopy (cryo-EM) as shown in FIG. 24, panel C. To verify that the encapsulated GFP-mRNA is taken up and expressed in cells, confocal imaging was performed in liver sinusoidal endothelial cells (LSECs). The results showed successful expression of the green fluorescent protein in LSEC cells, in contrast to the absence of fluorescent signal in cells incubated with poly-A encapsulating LNPs (see, e.g., FIG. 25). To also demonstrate in vivo expression of GFP-mRNA, we formulated an infrared fluorescence-labeled GFP-mRNA LNP that can be used for IVIS imaging and confocal microscopy. After i.v. injection of the DiR/GFP-mRNA LNP, the in vivo imaging system (IVIS) demonstrated a strong fluorescence signal in the abdominal region, corresponding to the location of the liver and spleen (see, e.g., FIGS. 25 and 26). All considered, these results show successful synthesis of an in-house LNP delivery platform, which can be used, to obtain GFP-mRNA encapsulation and delivery, with fluorescent protein expression in the liver and spleen.

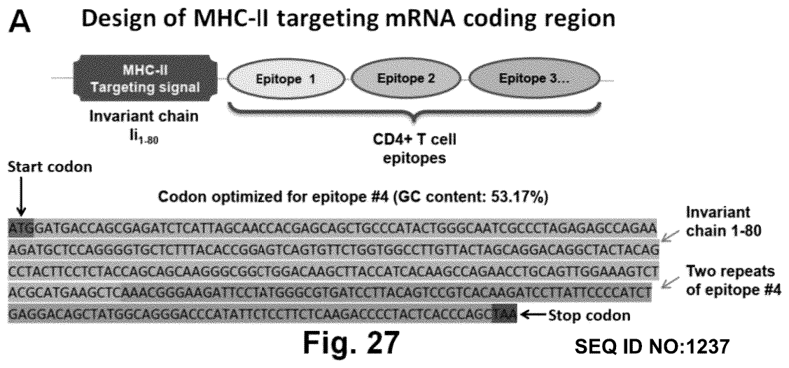

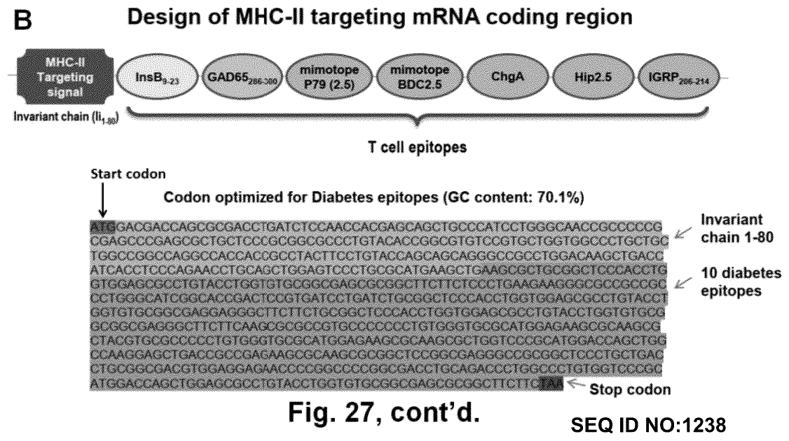

The above success allowed us to develop therapeutic LNPs for delivery of single- or multiepitope mRNA constructs to test tolerogenic effects, leading to prevention of peanut allergy anaphylaxis. As the lead example, we began constructing an mRNA LNP for expression of the Arah2 #4 epitope (see, FIG. 27). The codon-optimized version of this mRNA strand was synthesized by TRILINK®, followed by optimizing the synthesis parameters to obtain Arah2 #4-mRNA LNPs.

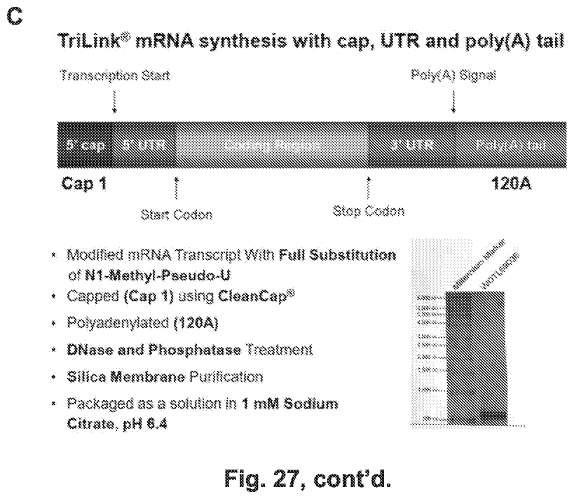

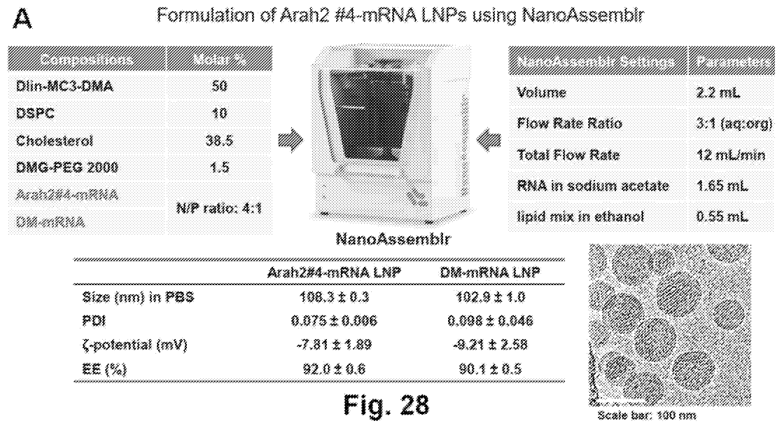

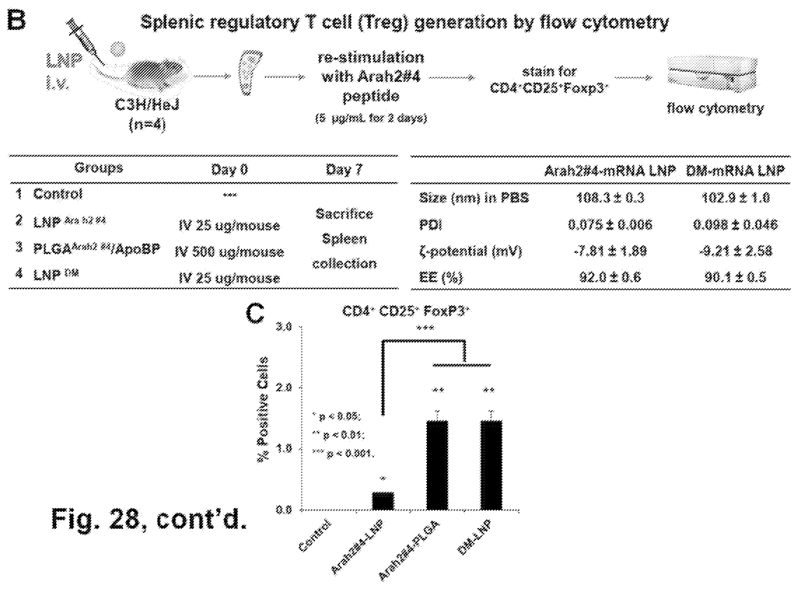

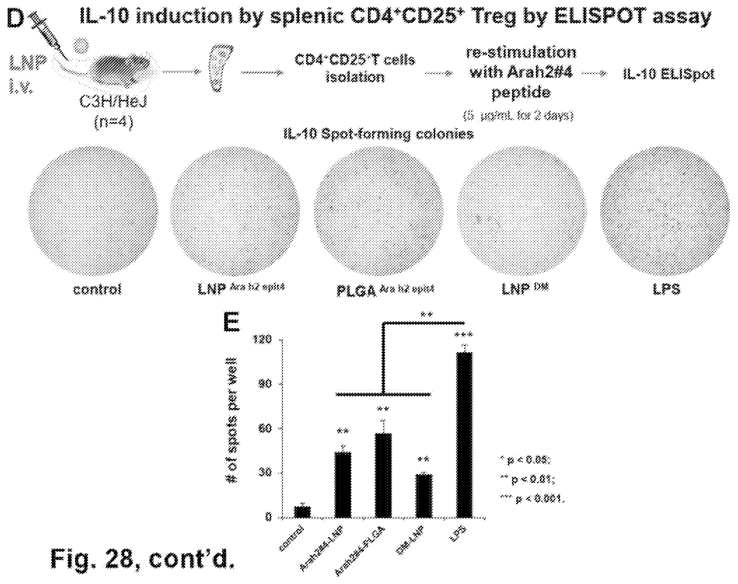

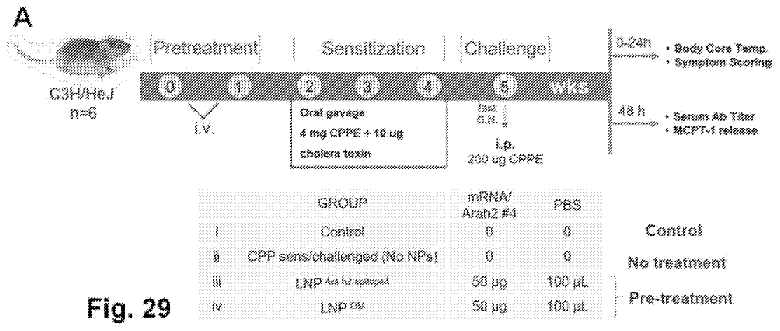

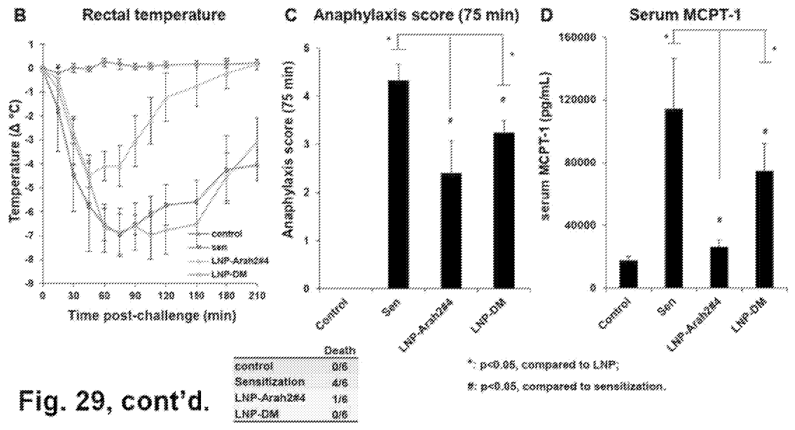

These particles were IV injected one week prior to obtaining the animal spleens for testing to see if the mRNA construct could induce regulatory T cells (Tregs) in vivo. Flow cytometry analysis showed that Arah2 #4-mRNA encapsulated LNP could induce a statistically significant increase in Fox P3⁺ Tregs, in comparison to untreated control mice. Treg harvesting and performance of ELISPOT assays demonstrated the successful generation of IL-10 producing cells, compared to non-treated controls (see, FIG. 28. This prompted further testing of the efficacy of Arah2 #4-mRNA LNPs in a peanut-induced anaphylaxis model. The results demonstrate that prior IV injection of the Arah2 #4-mRNA LNPs provides significant protection against the in vivo anaphylaxis manifestations, characterized as a drop of the core temperature, anaphylactic symptoms or animal death as well as mast cell protease-1 (MCPT-1) release in comparison to a control mRNA LNP (delivering an epitope sequence for treating a diabetic animal response) (see, e.g., FIG. 29). Thus, our data suggest the potential of therapeutic intervention in peanut anaphylaxis through the use of mRNA-LNPs.

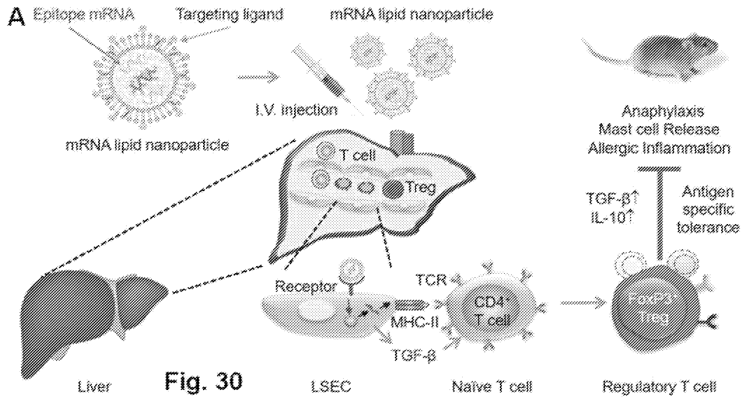

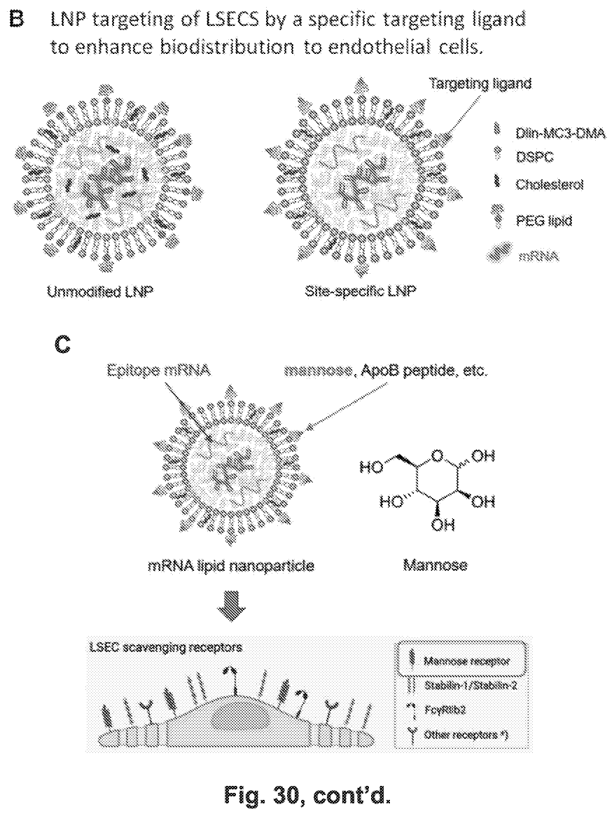

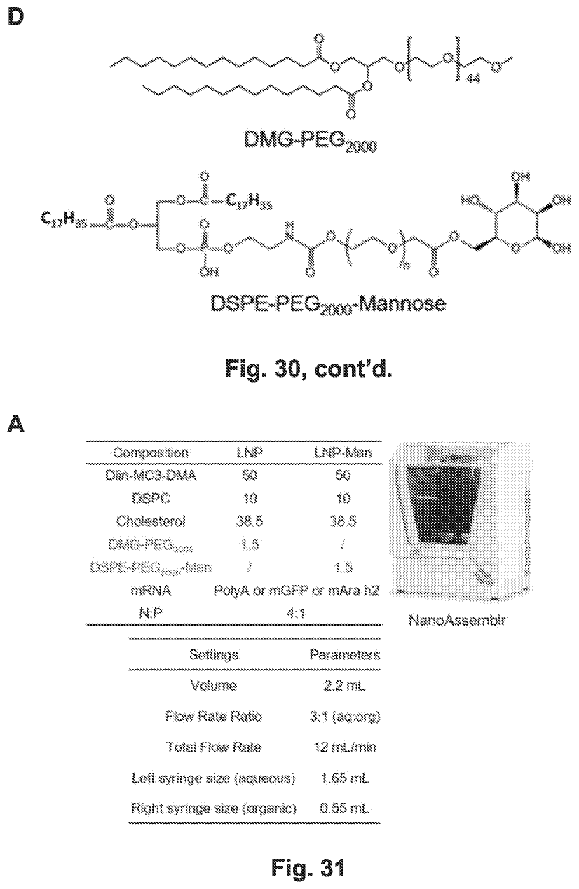

Above data demonstrate successful synthesis and delivery of GFP-mRNA lipid nanoparticles to liver. Noteworthy, GFP mRNA expression occurred in hepatocytes as well as sinusoidal lining cells, indicating that liver uptake is not preferential for LSECs, such as seen during the use ApoB peptide decorated PLGA nanoparticles. In order to see if we could improve GFP mRNA expression by LSECs targeting, we hypothesized that it should be possible to attach surface ligands to the LNPs for uptake by LSEC receptors (see, e.g., FIG. 30, panels A-D). There are several receptor types that are abundantly expressed on the LSEC surface, including the mannose receptor (also known as CD206) and the stabilin 1/stabilin 2 receptors. The ApoB peptide targets the stabilin receptor, allowing preferential PLGA nanoparticle uptake by LSECs, with the ability to deliver a peanut allergy epitope that prevents anaphylaxis by the crude peanut allergen extract. While it may be possible to covalently attach ApoB to the LNP surface (e.g., by using maleimide-conjugated DSPE-PEG), this would require a synthetic procedure that could add time and cost to mRNA-LNP synthesis. Moreover, due to the sensitivity of mRNA LNPs to degradation, it is not recommended that covalent coupling is used unless there is no alternative. For this purpose, we assessed the use of a LSEC targeting ligand that is already conjugated to a lipid component, for inclusion in our LPN synthesis protocol. To this end, we have previously demonstrated that mannan is a specific CD206 ligand and can be used for PLGA nanoparticle uptake in the liver, with successful generation of a tolerogenic response to ovalbumin.

Since DSPE-PEG-Mannose is commercially available, we experimented by replacing some of the DMG-PEG2000, included in the LNP formulation discussed above. This allows one-step synthesis of a mRNA-LNP that displays mannose on its surface, in addition to the possibility that the conjugated ligand could stabilize the association of mannose-conjugated PEG to the particle surface because of a longer carbon chain (18 instead of 14 carbons). This could be advantageous from the perspective that PEG reduces binding of the of ApoE lipoprotein to the LNP surface. ApoE is proposed to be a component of the LNP surface corona that allows particle uptake by hepatocytes. Thus, the reduction of ApoE binding together with expression of an LSEC ligand may favor LSEC uptake.

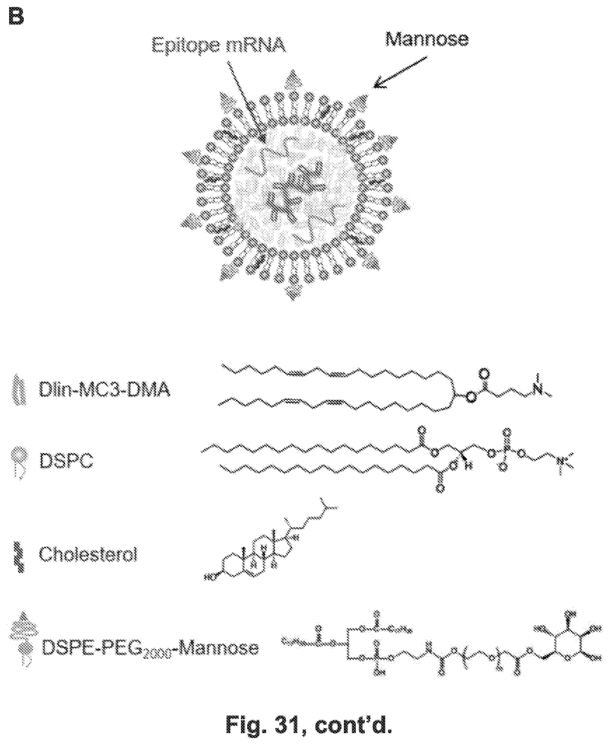

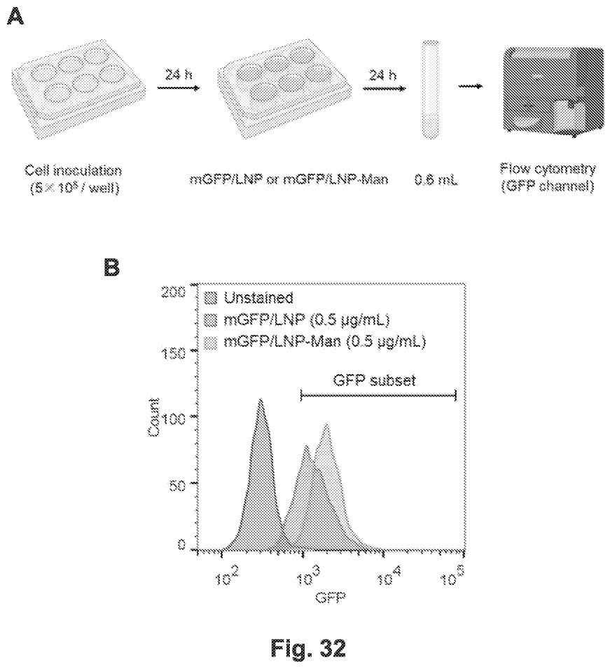

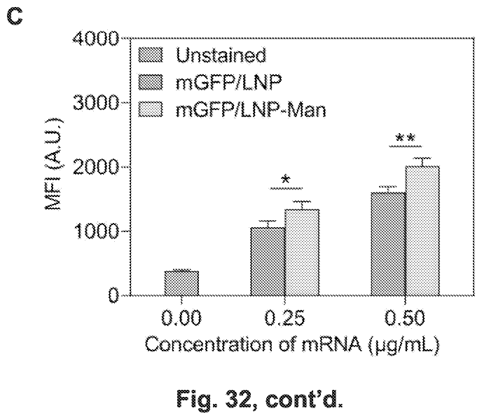

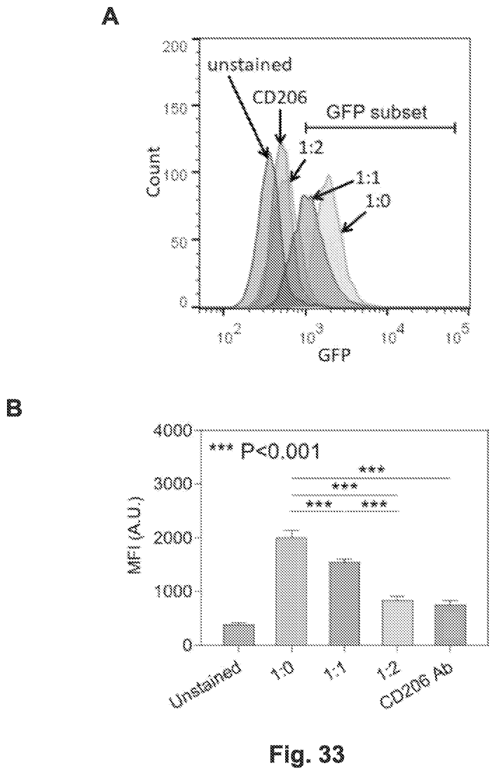

The microfluidics mixer (NanoAssemblr) was used to prepare the mGFP/LNP-Mannose according to the protocols outlined above (see also, FIG. 31, panels A-B). We demonstrate the achievement of a 90% encapsulation efficiency for GFP mRNA, yielding a particle of the size ˜ 150 nm and with a slight-negative surface charge of −5 mV. Moreover, assessment of cellular mRNA expression in a flow cytometry assay demonstrated that LSECs had significantly higher GFP expression than cells exposed to the non-decorated mGFP/LNP (see, e.g., FIG. 32, panels A-C).

In order to demonstrate that the LSEC uptake is ligand-specific, we performed a cellular study to demonstrate that an excess of PolyA/LNP-Mannose could compete for cellular uptake of the mannose-decorated GFP mRNA particle see, e.g., 33, panels A-B). The experimental design details are illustrated in Table 73.

TABLE 73
Details of the experimental design using non-GFP expressing
decorated PolyA.NLP-Man or anti-CD206 antibodies in
the presence of the decorated mGFP/LNP-Man.

| Groups | mGFP/LNP-Man | PolyA/LNP-Man | Anti-CD206 Ab |
|---|---|---|---|
| 1 (unstained) | / | / | / |
| 2 (1:0) | 0.5 μg/mL | / | / |
| 3 (1:1) | 0.5 μg/mL | 0.5 μg/mL | / |
| R (1:2) | 0.5 μg/mL | 1.0 μg/mL | / |
| 5 (CD206) | 0.5 μg/mL | / | 0.2 μg/mL |

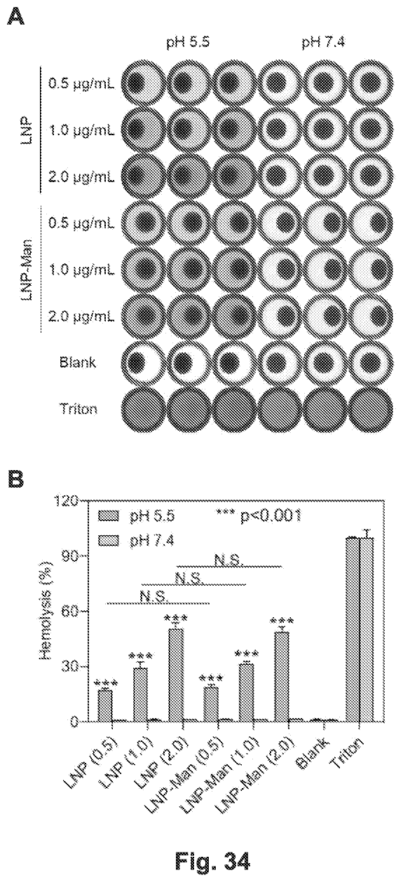

In addition, we showed that attachment of the ligand did not interfere in the hexagonal lipid phase mediated binding of the LNP to the membrane of red blood cells, which were chosen as a surrogate for studying membrane lysis under acidic but not neutral pH conditions. There was no difference in RBL lysis of decorated versus non-decorated particles under acidic conditions, suggesting that the surface ligand is unlikely to interfere in endosomal escape (see, e.g., FIG. 34, panels A-B).

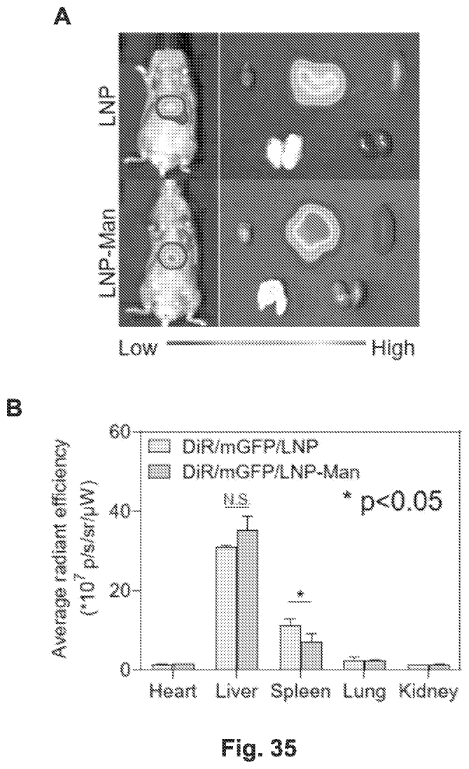

The next phase of experimentation was conducted in animals. Using the near-infrared fluorescent dye, DiR, for labeling mRNA/LNP-Man (DiR/mGFP/LNP-Man), we observed effective biodistribution to the liver by performing IVIS imaging after IV injection (see, FIG. 35, panels A-B). Characterization of DiR/mGFP/LNP and DiR/mGFP/LNP-Man is shown in Table 74.

TABLE 74
Characterization of DiR/mGFP/LNP and DiR/mGFP/LNP-Man.

| Characterization | DiR/mGFP/LNP | DiR/mGFP/LNP-Man |
|---|---|---|
| Size (nm) | 105.2 ± 2.7 | 140.5 ± 3.4 |
| Zeta (mV) | −0.58 | −4.02 |
| Polydispersity | 0.155 ± 0.021 | 0.197 ± 0.036 |
| EE % | 92.2% | 90.8% |

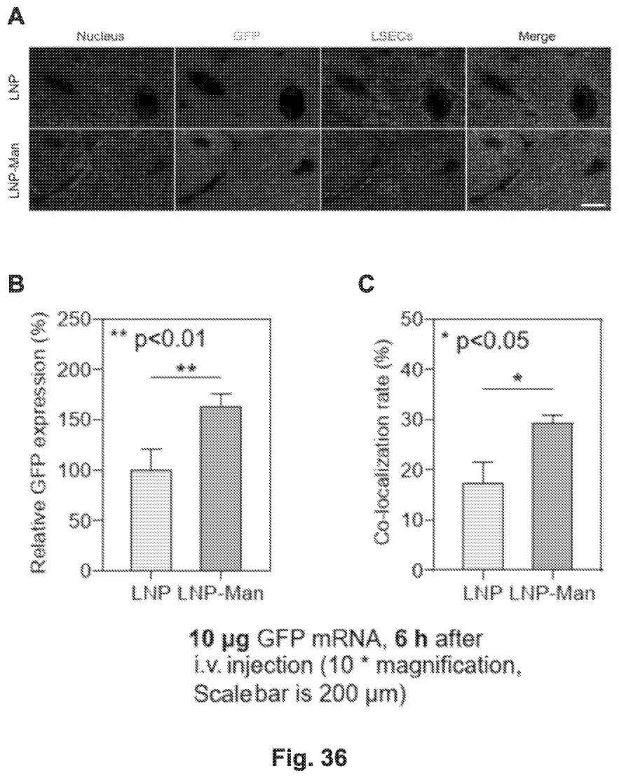

There were no significant differences in the in vivo versus ex vivo IVIS imaging results comparing decorated and non-decorated particles. However, confocal microscopy showed that more GFP was expressed in the liver of animals treated with decorated versus non-decorated particles, and that for the decorated particles, there was a higher co-localization index for decorated versus non-decorated particles in relation to an LSEC marker ((FIG. 36, panel A-B).

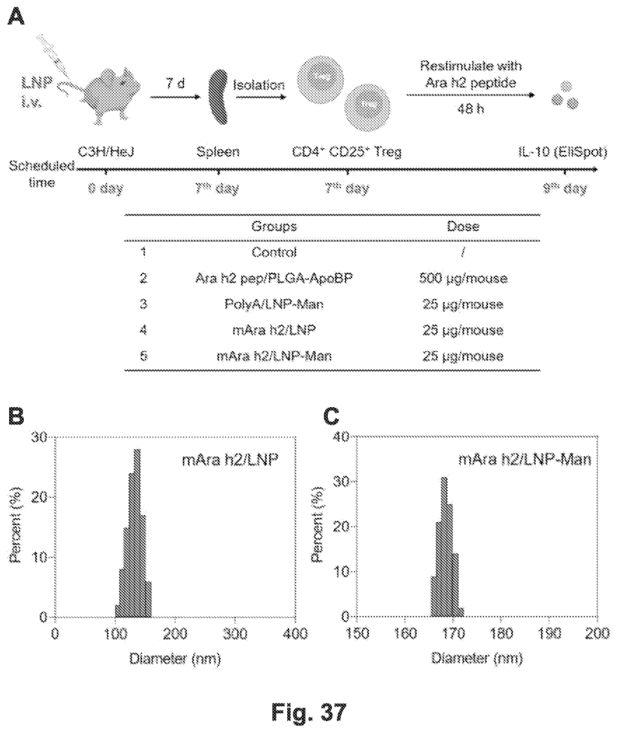

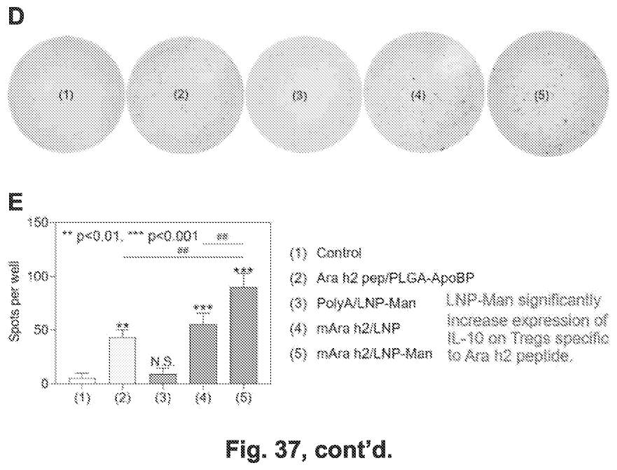

Based on these results, we synthesized a LNP-Man that encapsulates mRNA for the Ara h2 epitope 4 (mAra h2/LNP-Man) and then used these particles for IV injection and assessment of the generation of CD4.CD25.Foxp3. Tregs in the spleen, as well as testing IL-10 production in an ELISpot assay. Characteriation of the mAra h2/LNP and mAra h2/LNP-Man particles is shown in Table 75 and in FIG. 37, panels B and C.

TABLE 75
Characterization details for mAra h2/LNP and mAra h2/LNP-Man.

| Characterization | mAra h2/LNP | mAra h2/LNP-Man |
|---|---|---|
| Size (nm) | 120.1 ± 0.9 | 166.4 ± 1.9 |
| Zeta (mV) | −3.70 | −5.54 |
| Polydispersity | 0.195 ± 0.005 | 0.165 ± 0.029 |
| EE % | 92.7% | 91.5% |

We found mAra h2/LNP-Man induced significantly more Tregs than LNPs without mannose. The ELISpot results revealed increased IL-10 production by Tregs after treatment with mAra h2/LNP-Man (FIG. 37, panels D-E). Animal anaphylaxis studies are currently being undertaken to assess whether the increased Treg generation will impact the anaphylactic response to crude peanut extract.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. (canceled)

2. A tolerogenic nanoparticle comprising:
a nanoparticle comprising one or more lipid;
a nucleic acid encoding a peptide epitope comprising an antigen or antigen fragment to which immune tolerance is to be induced by administration of said tolerogenic nanoparticle to a mammal, where said peptide epitope comprises an epitope selected from the group consisting an epitope from a peanut allergen, an epitope from a house dust mite allergen, an adalimumab T cell epitope, a pancreatic beta cell autoantigen epitope, a histone epitope, and a viral vector T cell epitope, where said nucleic acid is encapsulated within or attached to the surface of said nanoparticle; and
wherein said nanoparticle comprises or consists of lipids that accumulate in the liver and/or said nanoparticle comprises a targeting moiety attached to the surface of said nanoparticle where said targeting moiety binds to a liver cell.

3. The tolerogenic nanoparticle of claim 2, wherein said nanoparticle further comprises one or more biocompatible polymers.

4.-10. (canceled)

11. The tolerogenic nanoparticle according to claim 2, wherein said tolerogenic nanoparticle comprises one or more cationic lipids.

12. The tolerogenic nanoparticle of claim 11, wherein said one or more cationic lipids is selected from the group consisting of dilinolcylmethyl-4 dimethyl aminobutyrate (DLin-MC3-DMA), 1,2-dioleoyl-3-dimethylaminopropane (DODAP), didodecyl-dimethylammonium bromide (DDAB), 1,2, dioleoyloxy-3-trimethylammonium propane chloride (DOTAP), (N-[1-(2.3-dioleyloxy)propyl]-N,N,N-trimethylammonium chloride (DOTMA), 1,2-dilinoleyloxy-3-dimethylaminopropane (DLinDMA), 2,2-dilinoleyl)-4-dimethylaminomethyl-[1,3]-dioxolane (DLin-K-DMA), 2,2-dilinoleyl-4-(2· dimethylaminoethyl)-(1,3)-dioxolane (DLin-KC2-DMA), C12-200 (Lipid 5), 3-(dimethylamino)propyl (12Z,15Z)-3-[(9Z,12Z)-octadeca-9,12-dien-1-yl]benicosa-12,15-dienoate (DMAP-BLP), and (2Z)-non-2-en-1-yl 10-[(Z)-(1-methylpiperidin-4 yl) carbonyloxy]nonadecanoate (L101), dicetylphosphate-tetraethylenepentaamine-based polycation lipid, YSK05, YSK12-C4, YSK13-C4, and YSK15-C4.

13. (canceled)

14. The tolerogenic nanoparticle of claim 2, wherein said nanoparticle comprises a helper lipid.

15. The tolerogenic nanoparticle of claim 14, wherein said helper lipid comprises a lipid selected from the group consisting of 1,2-distearoyl-sn-glycero-3-phosphocholine (DSPC), 1,2-dioleoyl-sn-glycero-3-phosphoethanolamine (DOPE), and 1-Palmitoyl-2-oleoyl-sn-glycero-3-phosphoethanolamine (POPE).

16. (canceled)

17. The tolerogenic nanoparticle according to claim 2, wherein said nanoparticle comprises cholesterol.

18. The tolerogenic nanoparticle according to claim 2, wherein said lipidic nanoparticle comprises a PEG-lipid.

19. (canceled)

20. The tolerogenic nanoparticles according to claim 2, wherein said nanoparticle comprises an ionizable lipid, a helper lipid, a PEG-lipid, and cholesterol.

21. The tolerogenic nanoparticle of claim 20, wherein said nanoparticle comprises DLin-MC3-DMA, DSPC, cholesterol, and a PEG lipid.

22.-36. (canceled)

37. The tolerogenic nanoparticle according to claim 2, wherein said nanoparticle ranges in size from about 50 am, or from about 100 nm, or from about 200 om up to about 450 nm, or up to about 400 nm, or up to about 350 nm, or up to about 300 nm.

38. The tolerogenic nanoparticle of claim 37, wherein said nanoparticle ranges in size from about 200 nm up to about 300 nm, or from about 100 nm up to about 170 nm, or from 100 om up to about 130 nm.

39. The tolerogenic nanoparticle according to claim 2, wherein said nanoparticle comprises said targeting moiety that binds to an APC displaying a scavenger receptor in the liver.

40. The tolerogenic nanoparticle of claim 39, wherein said targeting moiety binds to a liver sinusoidal endothelial cell (LSEC).

41. The tolerogenic nanoparticle of claim 39, wherein said targeting moiety binds to or more scavenger receptors selected from the group consisting of Stabilin 1, Stabilin 2, and a mannose receptor.

42. The tolerogenic nanoparticle of claim 41, wherein said targeting moiety comprises mannose.

43. The tolerogenic nanoparticle of claim 41, wherein said targeting moiety comprises a fragment of apolipoprotein B protein effective to bind to Stabilin 1 and/or Stabilin 2.

44. The tolerogenic nanoparticle of claim 43, wherein said targeting moiety fragment ranges in length from about 5, or from about 8, or from about 10 up to about 50, or up to about 40, or up to about 30, or up to about 20 amino acids.

45. The tolerogenic nanoparticle of claim 44, wherein said targeting moiety comprises a fragment of the apoB protein comprising the amino acid sequence RKRGLK (SEQ ID NO:18), RLYRKRGLK (SEQ ID NO: 19) or CGGKLGRKYRYLR (SEQ ID NO:4).

46.-47. (canceled)

48. The tolerogenic nanoparticle according to claim 2, wherein said targeting moiety is physically adsorbed to said nanoparticle.

49. The tolerogenic nanoparticle according to claim 2, wherein said targeting moiety is covalently bound to said nanoparticle directly or through a linker.

50.-52. (canceled)

53. The tolerogenic nanoparticle of claim 49, wherein said targeting moiety is bound to a lipid comprising said nanoparticle.

54. The tolerogenic nanoparticle of claim 53, wherein said lipid is a pegylated lipid, a cationic lipid, and/or to cholesterol and/or CHEMS.

55. The tolerogenic nanoparticle of claim 54, wherein said targeting moiety comprises mannose attached to a pegylated lipid.

56. The tolerogenic nanoparticle of claim 55, wherein said targeting moiety comprises DSPE-PEG-Man.

57. The tolerogenic nanoparticle according to claim 56, wherein said nanoparticle is a lipidic nanoparticle comprising Dlin-MC3-DMA, DSPC, Cholesterol, and DSPE-PEG-Man.

58. The tolerogenic nanoparticle according to claim 57, wherein the molar ratio of Dlin-MC3-DMA:DSPC:Cholesterol:DSPE-PEG-Man is 50:10:38.5:1.5.

59.-70. (canceled)

71. The tolerogenic nanoparticle according to claim 2, wherein said peptide epitope comprises a peptide epitope that shows affinity binding to HLA class II molecules.

72. (canceled)

73. The tolerogenic nanoparticle according to claim 2, wherein said nucleic acid encodes a peptide epitope comprising one or more peanut peptide epitopes selected from the epitopes shown in Table 2, and/or Table 3, and/or Table 4, and/or Table 5, and/or Table 6, and/or Table 7, and/or Table 8, and/or Table 9, and/or Table 10, and/or Table 11, and/or Table 12.

74.-75. (canceled)

76. The tolerogenic nanoparticle according to claim 73, wherein said tolerogenic nanoparticle comprises 1, 2, 3, 4, 5, 6, 7, 8, 9, 10, 11, 12, 13, 14, or 15 epitopes selected from the peanut allergen epitopes shown in Table 10; of said tolerogenic nanoparticle comprises a nucleic acid that encodes 1, 2, 3, 4, 5, 6, 7, 8, 9, 10, 11, 12, 13, 14, or 15 epitopes selected from the epitopes shown in Table 10.

77. The tolerogenic nanoparticle according to claim 73, wherein said tolerogenic nanoparticle comprises 1, 2, 3, 4, 5, 6, 7, 8, 9, or 10 epitopes selected from the peanut allergen epitopes in Tables 11 and 12 or said nucleic acid encodes 1, 2, 3, 4, 5, 6, 7, 8, 9, or 10 epitopes selected from the epitopes in Tables 11 and 12.

78. (canceled)

79. The tolerogenic nanoparticle according to claim 2, wherein tolerogenic nanoparticle comprises a nucleic acid that encodes a peptide epitope comprising one or more dust mite peptide epitopes selected from the epitopes shown in Table 18, and/or Table 19, and/or Table 20, and/or Table 21, and/or Table 22, and/or Table 23, and/or Table 24, and/or Table 25, and/or Table 26, and/or Table 27, and/or Table 28.

80.-83. (canceled)

84. The tolerogenic nanoparticle according to claim 2, wherein said tolerogenic nanoparticle comprises a nucleic acid encoding one or more epitopes from the adalimumab heavy chain and/or the adalimumab light chain.

85.-88. (canceled)

89. The tolerogenic nanoparticle according to claim 2, wherein said tolerogenic nanoparticle comprises a nucleic acid encoding one or more peptide epitopes for the treatment or prophylaxis of diabetes, selected from the beta-cell autoantigen epitopes shown in Table 50, and/or Table 51.

90. (canceled)

91. The tolerogenic nanoparticle of claim 89, wherein said tolerogenic nanoparticle comprises a nucleic acid encoding a plurality of peptide epitopes selected from the selected from the epitopes shown in Table 50, and/or Table 51.

92. The tolerogenic nanoparticle of claim 91, wherein said plurality of peptide epitopes comprises at least 2, 3, 4, 5, 6, 7, 8, 9, 10, 11, or 12 different epitopes.

93. The tolerogenic nanoparticle according to claim 2, wherein said tolerogenic nanoparticle comprises a nucleic acid encoding one or more histone autoantigen epitopes selected from the epitopes shown in Table 54.

94.-96. (canceled)

97. The tolerogenic nanoparticles according to claim 2, wherein said tolerogenic nanoparticles comprises a nucleic acid encoding one or more AAV peptide epitopes, selected from the epitopes shown in Table 59, and/or Table 60, and/or Table 61.

98.-100. (canceled)

101. The tolerogenic nanoparticle according to claim 2, wherein said nucleic acid that encodes one or more epitopes is an mRNA.

102. The tolerogenic nanoparticle of claim 101, wherein mRNA coding sequence is further modified by a non-natural RNA nucleobase Nl-methylpseudouridine (m 1 Y).

103. The tolerogenic nanoparticle according to claim 2, wherein said nucleic acid that encodes one or more epitopes is a DNA.

104. The tolerogenic nanoparticle according to claim 101, wherein the N/P ratio of said nanoparticle ranges from 1:1 to about 10:1, or from about 2:1 to about 6:1, or from about 3:1 to about 5:1.

105. The tolerogenic nanoparticle of claim 104, wherein the N/P ratio of said nanoparticle is about 4:1.

106. The tolerogenic nanoparticle according to claim 2, wherein when said nucleic acid encodes a plurality of peptide epitopes, said nucleic acid encoding linker sequence(s) joining said peptide epitopes.

107. The tolerogenic nanoparticle of claim 106, wherein said linker sequence(s) comprise a furin cleavage site, a cathepsin cleavage site or a cathepsin L cleavage site.

108.-109. (canceled)

110. The tolerogenic nanoparticle according to claim 2, where said nucleic acid encodes peptide epitope(s) fused to an amino acid sequence that accesses trans-Golgi vesicles that transport type II MHC gene products to the cell surface.

111. The tolerogenic nanoparticle of claim 110, wherein said amino acid sequence comprises a transferrin receptor I transmembrane domain.

112. The tolerogenic nanoparticle of claim 110, wherein said amino acid sequence comprises a TIR 1.118 domain or the li-chain (114 aa).

113. The tolerogenic nanoparticle according to claim 2, said nucleic acid encodes peptide epitope(s) in which one or more or all cysteines are replaced with serines.

114. A pharmaceutical formulation, said formulation comprising: a tolerogenic nanoparticle according to claim 2; and a pharmaceutically acceptable carrier.

115. The pharmaceutical formulation of claim 114, wherein said formulation is a unit dosage formulation.

116. The pharmaceutical formulation according to claim 114, wherein said formulation is formulated for administration via a route selected from the group consisting of oral administration, inhalation, nasal administration, rectal administration, intraperitoneal injection, intravascular injection, subcutaneous injection, transcutaneous administration, intrathecal administration and intramuscular injection.

117. A method for the treatment and/or prophylaxis of peanut allergy in a mammal, said method comprising: administering to said mammal an effective amount of a tolerogenic nanoparticle according to claim 73.

118. A method for the treatment and/or prophylaxis of a dust mite allergy in a mammal, said method comprising: administering to said mammal an effective amount of a tolerogenic nanoparticle according to claim 79.

119. A method for the suppressing or preventing an immune response to adalimumab in a mammal, said method comprising: administering to said mammal an effective amount of a tolerogenic nanoparticle according to claim 84.

120. A method for the treatment and/or prophylaxis of type I diabetes in a mammal, said method comprising: administering to said mammal an effective amount of a tolerogenic nanoparticle according to claim 89.

121. A method for the treatment and/or prophylaxis of lupus in a mammal, said method comprising: administering to said mammal an effective amount of a tolerogenic nanoparticle according to claim 93.

122. A method for the suppressing or preventing an immune response to an AAV viral vector in a mammal, said method comprising: administering to said mammal an effective amount of a tolerogenic nanoparticle according to claim 97.

123.-124. (canceled)

125. The method according to claim 117, wherein said mammal is a human.

126. (canceled)

127. The method according to claim 117, wherein said nanoparticle contains an immune modulator.

128. The method of claim 127, wherein said immune modulator comprises rapamycin or a rapamycin analog.

129. (canceled)

130. The method of claim 128, wherein said rapamycin analog is selected from the group consisting of temsirolimus, everolimus, and ridaforolimus.
